(19) United States
(12) Patent Application Publication
(10) Pub. No.: US 2023/0340485 A1
(43) Pub. Date: Oct. 26, 2023

(54) ANAPLASTIC LYMPHOMA KINASE (ALK) iRNA AGENT COMPOSITIONS AND METHODS OF USE THEREOF

(71) Applicant: ALNYLAM PHARMACEUTICALS, INC., CAMBRIDGE, MA (US)

(72) Inventors: BRET L. BOSTWICK, CAMBRIDGE, MA (US); JAMES D. MCININCH, BURLINGTON, MA (US)

(73) Assignee: ALNYLAM PHARMACEUTICALS, INC., CAMBRIDGE, MA (US)

(21) Appl. No.: 18/001,813

(22) PCT Filed: Jun. 15, 2021
(86) PCT No.: PCT/US2021/037437

§ 371 Date: Dec. 14, 2022

Related U.S. Application Data
(60) Provisional application No. 63/140,532, filed on Jan. 22, 2021.
(60) Provisional application No. 63/039,897, filed on Jun. 16, 2020.

(51) Int. Cl. C12N 15/113; A61P 3/00

(52) U.S. Cl. CPC C12N 15/1137; A61P 3/00; C12N 2310/315; C12N 2310/32; C12N 2310/351

(57) ABSTRACT

The disclosure relates to double stranded ribonucleic acid (dsRNAi) agents and compositions targeting an anaplastic lymphoma kinase (ALK) gene, as well as methods of inhibiting expression of an ALK gene and methods of treating subjects having an ALK-associated disease or disorder, e.g., type 2 diabetes, obesity, or an obesity-associated disorder, using such dsRNAi agents and compositions.

### CROSS-REFERENCE TO RELATED APPLICATIONS

This application claims the benefit of priority to U.S. Provisional Application No. 63/039,897, filed on Jun. 16, 2020, and claims the benefit of priority to U.S. Provisional Application No. 63/140,532, filed on Jan. 22, 2021. The entire contents of the foregoing applications are hereby incorporated herein by reference.

### SEQUENCE LISTING

The instant application contains a Sequence Listing which has been submitted electronically in ASCII format and is hereby incorporated by reference in its entirety. Said ASCII copy, created on May 26, 2021, is named A108868_1140WO_SL.txt and is 470,114 bytes in size.

### BACKGROUND OF THE INVENTION

The anaplastic lymphoma kinase (ALK) gene encoding the anaplastic lymphoma kinase protein is located in the chromosomal regions 2p23.2-p23.1 on chromosome 2 and consists of 29 exons. The ALK protein is a receptor tyrosine kinase and belongs to the insulin receptor superfamily.

Variants within the ALK gene have been identified to associate with the thinness (low body mass index) phenotype in humans. In mice, genetic deletion of ALK resulted in thin animals with increased energy consumption and resistance to diet- or leptin mutation-induced obesity. In drosophila, knockdown of ALK expression led to decreased triglyceride levels. Studies have indicated that ALK expression in the brain controls energy expenditure of the body via sympathetic control of adipose tissue lipolysis. Therefore, interference with ALK activity would promote weight loss and treat obesity in humans.

The prevalence of obesity and obesity-associated complications continue to rise throughout the world. Obesity is the major risk factor for serious health conditions such as type 2 diabetes, coronary heart disease, stroke, osteoarthritis, sleep apnea, and various types of cancer. Currently available methods for promoting weight loss, e.g., reducing calorie intake and increasing exercise, have yielded limited success due to the slow process, poor compliance, or regaining of lost weight after completion of the weight loss intervention.

Accordingly, there is a need for improved methods of promoting weight loss and supporting weight maintenance following weight loss, including agents that can selectively and efficiently inhibit the ALK gene, such that subjects having type 2 diabetes, obesity, or an obesity-associated disorder, e.g., coronary heart disease, can be effectively treated.

### BRIEF SUMMARY OF THE INVENTION

The present disclosure provides RNAi compositions, which effect the RNA-induced silencing complex (RISC)-mediated cleavage of RNA transcripts of an ALK gene. The ALK gene may be within a cell, e.g., a cell within a subject, such as a human. The use of these iRNAs enables the targeted degradation of mRNAs of the corresponding gene (ALK gene) in mammals.

The iRNAs of the invention have been designed to target an ALK gene, e.g., an ALK wild type gene and/or an ALK gene variant. The iRNAs of the invention inhibit the expression of the ALK gene by at least about 30%, 35%, 40%, 45%, 50%, 55%, 60%, 65%, 70%, 75%, 80%, 85%, 90%, 95%, or 99%. Without intending to be limited by theory, it is believed that a combination or sub-combination of the foregoing properties and the specific target sites, or the specific modifications in these iRNAs confer to the iRNAs of the invention improved efficacy, stability, potency, durability, and safety. In one aspect, the present invention provides double stranded ribonucleic acid (dsRNA) agent for inhibiting expression of ALK, wherein the dsRNA agent comprises a sense strand and an antisense strand forming a double stranded region, wherein the sense strand comprises at least 15 contiguous nucleotides differing by no more than 3 nucleotides from the nucleotide sequence of SEQ ID NO: 1 and the antisense strand comprises at least 15 contiguous nucleotides differing by no more than 3 nucleotides from the nucleotide sequence of SEQ ID NO: 2.

In another aspect, the present invention provides a double stranded ribonucleic acid (dsRNA) agent for inhibiting expression of ALK, wherein the dsRNA agent comprises a sense strand and an antisense strand forming a double stranded region, wherein the antisense strand comprises a region of complementarity to an mRNA encoding ALK, and wherein the region of complementarity comprises at least 15 contiguous nucleotides differing by no more than 3 nucleotides from the nucleotide sequence of SEQ ID NO:2.

In yet another aspect, the present invention provides a double stranded ribonucleic acid (dsRNA) agent for inhibiting expression of ALK, wherein the dsRNA agent comprises a sense strand and an antisense strand forming a double stranded region, wherein the antisense strand comprises a region of complementarity to an mRNA encoding ALK, and wherein the region of complementarity comprises at least 15 contiguous nucleotides differing by no more than 3 nucleotides from any one of the antisense nucleotide sequences in Tables 2, 3, or 4.

In one embodiment, the sense strand comprises at least 15 contiguous nucleotides differing by no more than three nucleotides from any one of the nucleotide sequence of nucleotides 24-44, 57-77, 157-177, 245-265, 286-306, 314-334, 326-346, 343-363, 366-386, 382-402, 430-450, 448-468, 465-485, 499-519, 512-532, 587-607, 599-619, 612-632, 624-644, 636-656, 688-708, 702-722, 714-734, 754-774, 774-794, 798-818, 813-833, 827-847, 847-867, 859-879, 873-893, 919-939, 955-975, 987-1007, 1079-1099, 1100-1120, 1112-1132, 1124-1144, 1136-1156, 1148-1168, 1160-1180, 1183-1203, 1318-1338, 1361-1381, 1373-1393, 1394-1414, 1445-1465, 1462-1482, 1477-1497, 1499-1519, 1511-1531, 1524-1544, 1546-1566, 1560-1580, 1594-1614, 1616-1636, 1629-1649, 1644-1664, 1659-1679, 1672-1692, 1684-1704, 1697-1717, 1709-1729, 1724-1744, 1744-1764, 1787-1807, 1801-1821, 1847-1867, 1879-1899, 1895-1915, 1907-1927, 1930-1950, 1942-1962, 1954-1974, 1972-1992, 1985-2005, 2000-2020, 2023-2043, 2063-2083, 2075-2095, 2094-2114, 2107-2127, 2119-2139, 2138-2158, 2152-2172, 2164-2184, 2176-2196, 2196-2216, 2215-2235, 2246-2266, 2269-2289, 2291-2311, 2314-2334, 2327-2347, 2339-2359, 2354-2374, 2366-2386, 2390-2410, 2402-2422, 2420-2440, 2444-2464, 2464-2484, 2477-2497, 2489-2509, 2503-2523, 2534-2554, 2556-2576, 2575-2595, 2587-2607, 2599-2619, 2612-2632, 2637-2657, 2651-2671, 2672-2692, 2684-2704, 2707-2727, 2720-2740, 2732-2752, 2753-2773, 2773-2793, 2806-2826, 2819-2839, 2831-2851, 2843-2863, 2855-2875, 2875-2895, 2889-2909, 2901-2921, 2914-2934, 2929-2949, 2954-2974, 2980-3000, 2992-3012, 3047-3067, 3064-3084, 3076-3096, 3118-3138, 3130-3150, 3142-3162, 3159-3179, 3179-3199, 3191-3211, 3205-3225, 3218-3238, 3233-3253, 3251-3271, 3263-3283, 3275-3295, 3305-3325, 3317-3337, 3332-3352, 3347-3367, 3365-3385, 3378-3398, 3424-3444, 3436-3456, 3464-3484, 3504-3524, 3526-3546, 3538-3558, 3556-3576, 3570-3590, 3585-3605, 3599-3619, 3611-3631, 3625-3645, 3639-3659, 3651-3671, 3751-3771, 3774-3794, 3818-3838, 3834-3854, 3855-3875, 3895-3915, 3911-3931, 3926-3946, 3946-3966, 3959-3979, 3971-3991, 3991-4011, 4007-4027, 4037-4057, 4052-4072, 4064-4084, 4097-4117, 4109-4129, 4127-4147, 4139-4159, 4151-4171, 4163-4183, 4178-4198, 4198-4218, 4211-4231, 4234-4254, 4246-4266, 4259-4279, 4275-4295, 4337-4357, 4354-4374, 4394-4414, 4416-4436, 4429-4449, 4442-4462, 4459-4479, 4474-4494, 4526-4546, 4558-4578, 4619-4639, 4642-4662, 4656-4676, 4671-4691, 4693-4713, 4705-4725, 4717-4737, 4734-4754, 4747-4767, 4762-4782, 4774-4794, 4792-4812, 4807-4827, 4820-4840, 4847-4867, 4859-4879, 4871-4891, 4886-4906, 4901-4921, 4913-4933, 4948-4968, 4960-4980, 4972-4992, 5000-5020, 5012-5032, 5024-5044, 5046-5066, 5058-5078, 5075-5095, 5092-5112, 5111-5131, 5123-5143, 5135-5155, 5147-5167, 5159-5179, 5173-5193, 5190-5210, 5216-5236, 5229-5249, 5281-5301, 5293-5313, 5307-5327, 5327-5347, 5339-5359, 5378-5398, 5392-5412, 5405-5425, 5428-5448, 5449-5469, 5461-5481, 5477-5497, 5509-5529, 5523-5543, 5559-5579, 5576-5596, 5614-5634, 5633-5653, 5645-5665, 5657-5677, 5671-5691, 5686-5706, 5705-5725, 5723-5743, 5749-5769, 5761-5781, 5773-5793, 5787-5807, 5814-5834, 5826-5846, 5845-5865, 5857-5877, 5869-5889, 5881-5901, 5897-5917, 5909-5929, 5924-5944, 5956-5976, 5972-5992, 5984-6004, 5997-6017, 6032-6052, 6044-6064, 6056-6076, 6079-6099, 6097-6117, 6110-6130, 6128-6148, 6140-6160, 6153-6173, 6183-6203, 6203-6223, 6234-6254, or 6247-6267 of SEQ ID NO: 1, and the antisense strand comprises at least 15 contiguous nucleotides from the corresponding nucleotide sequence of SEQ ID NO: 2.

In one embodiment, the antisense strand comprises at least 15 contiguous nucleotides differing by no more than three nucleotides from any one of the antisense strand nucleotide sequences of a duplex selected from the group consisting of AD-1289971, AD-1289972, AD-1289973, AD-1289974, AD-1289975, AD-1289976, AD-1289977, AD-1289978, AD-1289979, AD-1289980, AD-1289981, AD-1289982, AD-1289983, AD-1289984, AD-1289985, AD-1289986, AD-1289987, AD-1289988, AD-1289989, AD-1289990, AD-1289991, AD-1289992, AD-1289993, AD-1289994, AD-1289995, AD-1289996, AD-1289997, AD-1289998, AD-1289999, AD-1290000, AD-1290001, AD-1290002, AD-1290003, AD-1290004, AD-1290005, AD-1290006, AD-1290007, AD-1290008, AD-1290009, AD-1290010, AD-1290011, AD-1290012, AD-1290013, AD-1290014, AD-1290015, AD-1290016, AD-1290017, AD-1290018, AD-1290019, AD-1290020, AD-1290021, AD-1290022, AD-1290023, AD-1290024, AD-1290025, AD-1290026, AD-1290027, AD-1290028, AD-1290029, AD-1290030, AD-1290031, AD-1290032, AD-1290033, AD-1290034, AD-1290035, AD-1290036, AD-1290037, AD-1290038, AD-1290039, AD-1290040, AD-1290041, AD-1290042, AD-1290043, AD-1290044, AD-1290045, AD-1290046, AD-1290047, AD-1290048, AD-1290049, AD-1290050, AD-1290051, AD-1290052, AD-1290053, AD-1290054, AD-1290055, AD-1290056, AD-1290057, AD-1290058, AD-1290059, AD-1290060, AD-1290061, AD-1290062, AD-1290063, AD-1290064, AD-1290065, AD-1290066, AD-1290067, AD-1290068, AD-1290069, AD-1290070, AD-1290071, AD-1290072, AD-1290073, AD-1290074, AD-1290075, AD-1290076, AD-1290077, AD-1290078, AD-1290079, AD-1290080, AD-1290081, AD-1290082, AD-1290083, AD-1290084, AD-1290085, AD-1290086, AD-1290087, AD-1290088, AD-1290089, AD-1290090, AD-1290091, AD-1290092, AD-1290093, AD-1290094, AD-1290095, AD-1290096, AD-1290097, AD-1290098, AD-1290099, AD-1290100, AD-1290101, AD-1290102, AD-1290103, AD-1290104, AD-1290105, AD-1290106, AD-1290107, AD-1290108, AD-1290109, AD-1290110, AD-1290111, AD-1290112, AD-1290113, AD-1290114, AD-1290115, AD-1290116, AD-1290117, AD-1290118, AD-1290119, AD-1290120, AD-1290121, AD-1290122, AD-1290123, AD-1290124, AD-1290125, AD-1290126, AD-1290127, AD-1290128, AD-1290129, AD-1290130, AD-1290131, AD-1290132, AD-1290133, AD-1290134, AD-1290135, AD-1290136, AD-1290137, AD-1290138, AD-1290139, AD-1290140, AD-1290141, AD-1290142, AD-1290143, AD-1290144, AD-1290145, AD-1290146, AD-1290147, AD-1290148, AD-1290149, AD-1290150, AD-1290151, AD-1290152, AD-1290153, AD-1290154, AD-1290155, AD-1290156, AD-1290157, AD-1290158, AD-1290159, AD-1290160, AD-1290161, AD-1290162, AD-1290163, AD-1290164, AD-1290165, AD-1290166, AD-1290167, AD-1290168, AD-1290169, AD-1290170, AD-1290171, AD-1290172, AD-1290173, AD-1290174, AD-1290175, AD-1290176, AD-1290177, AD-1290178, AD-1290179, AD-1290180, AD-1290181, AD-1290182, AD-1290183, AD-1290184, AD-1290185, AD-1290186, AD-1290187, AD-1290188, AD-1290189, AD-1290190, AD-1290191, AD-1290192, AD-1290193, AD-1290194, AD-1290195, AD-1290196, AD-1290197, AD-1290198, AD-1290199, AD-1290200, AD-1290201, AD-1290202, AD-1290203, AD-1290204, AD-1290205, AD-1290206, AD-1290207, AD-1290208, AD-1290209, AD-1290210, AD-1290211, AD-1290212, AD-1290213, AD-1290214, AD-1290215, AD-1290216, AD-1290217, AD-1290218, AD-1290219, AD-1290220, AD-1290221, AD-1290222, AD-1290223, AD-1290224, AD-1290225, AD-1290226, AD-1290227, AD-1290228, AD-1290229, AD-1290230, AD-1290231, AD-1290232, AD-1290233, AD-1290234, AD-1290235, AD-1290236, AD-1290237, AD-1290238, AD-1290239, AD-1290240, AD-1290241, AD-1290242, AD-1290243, AD-1290244, AD-1290245, AD-1290246, AD-1290247, AD-1290248, AD-1290249, AD-1290250, AD-1290251, AD-1290252, AD-1290253, AD-1290254, AD-1290255, AD-1290256, AD-1290257, AD-1290258, AD-1290259, AD-1290260, AD-1290261, AD-1290262, AD-1290263, AD-1290264, AD-1290265, AD-1290266, AD-1290267, AD-1290268, AD-1290269, AD-1290270, AD-1334980, AD-1334981, AD-1334982, AD-1334983, AD-1334984, AD-1334985, AD-1334986, AD-1334987, AD-1334988, AD-1334989, AD-1334990, AD-1334991, AD-1334992, AD-1334993, AD-1334994, AD-1334995, AD-1334996, AD-1334997, AD-1334998, AD-1334999, AD-1335000, AD-1335001, AD-1335002, AD-1335003, AD-1335004, AD-1335005, AD-1335006, AD-1335007, AD-1335008, AD-1335009, AD-1335010, AD-1335011, AD-1335012, AD-1335013, AD-1335014, AD-1335015, AD-1335016, AD-1335017, AD-1335018, AD-1335019, AD-1335020, AD-1335021, AD-1335022, AD-1335023, AD-1335024, AD-1335025, AD-1335026, AD-1335027, AD-1335028, AD-1335029, AD-1335030, AD-1335031, AD-1335032, AD-1335033, AD-1335034, AD-1335035, AD-1335036, AD-1335037, AD-1335038, AD-1335039, AD-1335040, AD-1335041, AD-1335042, AD-1335043, AD-1335044, AD-1335045, AD-1335046, AD-1335047, AD-1335048, AD-1335049, AD-1335050, AD-1335051, AD-1335052, AD-1335053, AD-1335054, AD-1335055, AD-1335056, AD-1335057, AD-1335058, AD-1335059, AD-1335060, AD-1335061, AD-1335062, AD-1335063, AD-1335064, AD-1335065, AD-1335066, AD-1335067, AD-1335068, AD-1335069, AD-1335070, AD-1335071, AD-1335072, AD-1335073, AD-1335074, AD-1335075, AD-1335076, AD-1335077, AD-1335078, AD-1335079, AD-1335080, AD-1335081, AD-1335082, AD-1335083, AD-1335084, AD-1335085, AD-1335086, AD-1335087, AD-1335088, AD-1335089, AD-1335090, AD-1335091, AD-1335092, AD-1335093, AD-1335094, AD-1335095, AD-1335096, AD-1335097, AD-1335098, AD-1335099, AD-1335100, AD-1335101, AD-1335102, AD-1335103, AD-1335104, AD-1335105, AD-1335106, AD-1335107, AD-1335108, AD-1335109, AD-1335110, AD-1335111, AD-1335112, AD-1335113, AD-1335114, AD-1335115, AD-1335116, AD-1335117, AD-1335118, AD-1335119, AD-1335120, AD-1335121, AD-1335122, AD-1335123, AD-1335124, AD-1335125, AD-1335126, AD-1335127, AD-1335128, AD-1335129, AD-1335130, AD-1335131, AD-1335132, AD-1335133, AD-1335134, AD-1335135, AD-1335136, AD-1335137, AD-1335138, AD-1335139, AD-1335140, AD-1335141, AD-1335142, AD-1335143, AD-1335144, AD-1335145, AD-1335146, AD-1335147, AD-1335148, AD-1335149, AD-1335150, AD-1335151, AD-1335152, AD-1335153, AD-1335154, AD-1335155, AD-1335156, AD-1335157, AD-1335158, AD-1335159, AD-1335160, AD-1335161, AD-1335162, AD-1335163, AD-1335164, AD-1335165, AD-1335166, AD-1335167, AD-1335168, AD-1335169, AD-1335170, AD-1335171, AD-1335172, AD-1335173, AD-1335174, AD-1335175, AD-1335176, AD-1335177, AD-1335178, AD-1335179, AD-1335180, AD-1335181, AD-1335182, AD-1335183, AD-1335184, AD-1335185, AD-1335186, AD-1335187, AD-1335188, AD-1335189, AD-1335190, AD-1335191, AD-1335192, AD-1335193, AD-1335194, AD-1335195, AD-1335196, AD-1335197, AD-1335198, AD-1335199, AD-1335200, AD-1335201, AD-1335202, AD-1335203, AD-1335204, AD-1335205, AD-1335206, AD-1335207, AD-1335208, AD-1335209, AD-1335210, AD-1335211, AD-1335212, AD-1335213, AD-1335214, AD-1335215, AD-1335216, AD-1335217, AD-1335218, AD-1335219, AD-1335220, AD-1335221, AD-1335222, AD-1335223, AD-1335224, AD-1335225, AD-1335226, AD-1335227, AD-1335228, AD-1335229, AD-1335230, AD-1335231, AD-1335232, AD-1335233, AD-1335234, AD-1335235, AD-1335236, AD-1335237, AD-1335238, AD-1335239, AD-1335240, AD-1335241, AD-1335242, AD-1335243, AD-1335244, AD-1335245, AD-1335246, AD-1335247, AD-1335248, AD-1335249, AD-1335250, AD-1335251, AD-1335252, AD-1335253, AD-1335254, AD-1335255, AD-1335256, AD-1335257, AD-1335258, AD-1335259, AD-1335260, AD-1335261, AD-1335262, AD-1335263, AD-1335264, AD-1335265, AD-1335266, AD-1335267, AD-1335268, AD-1335269, AD-1335270, AD-1335271, AD-1335272, AD-1335273, AD-1335274, AD-1335275, AD-1335276, AD-1335277, AD-1335278, AD-1335279.

In some embodiments, the nucleotide sequence of the sense and antisense strand comprises any one of the sense strand nucleotide sequences in Tables 2, 3, or 4.

In one embodiment, the sense strand, the antisense strand, or both the sense strand and the antisense strand is conjugated to one or more lipophilic moieties.

In one embodiment, the lipophilic moiety is conjugated to one or more internal positions in the double stranded region of the dsRNA agent.

In one embodiment, the lipophilic moiety is conjugated via a linker or carrier.

In one embodiment, the lipophilicity of the lipophilic moiety, measured by logKow, exceeds 0.

In one embodiment, the hydrophobicity of the double-stranded RNAi agent, measured by the unbound fraction in a plasma protein binding assay of the double-stranded RNAi agent, exceeds 0.2.

In one embodiment, the plasma protein binding assay is an electrophoretic mobility shift assay using human serum albumin protein.

In some embodiments, the dsRNA agent comprises at least one modified nucleotide.

In one embodiment, no more than five of the sense strand nucleotides and no more than five of the nucleotides of the antisense strand are unmodified nucleotides

In one embodiment, all of the nucleotides of the sense strand and all of the nucleotides of the antisense strand are modified nucleotides.

In one embodiment, at least one of the modified nucleotides is selected from the group a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a 5′-phosphorothioate group, a nucleotide comprising a 5′-methylphosphonate group, a nucleotide comprising a 5′ phosphate or 5′ phosphate mimic, a nucleotide comprising vinyl phosphonate, a nucleotide comprising adenosine-glycol nucleic acid (GNA), a nucleotide comprising thymidine-glycol nucleic acid (GNA) S-Isomer, a nucleotide comprising 2-hydroxymethyl-tetrahydrofurane-5-phosphate, a nucleotide comprising 2′-deoxythymidine-3′phosphate, a nucleotide comprising 2′-deoxyguanosine-3′-phosphate, and a terminal nucleotide linked to a cholesteryl derivative and a dodecanoic acid bisdecylamide group; and combinations thereof.

In one embodiment, the modified nucleotide is selected from the group consisting of a 2′-deoxy-2′-fluoro modified nucleotide, a 2′-deoxy-modified nucleotide, 3′-terminal deoxy-thymine nucleotides (dT), a locked nucleotide, an abasic nucleotide, a 2′-amino-modified nucleotide, a 2′-alkyl-modified nucleotide, a morpholino nucleotide, a phosphoramidate, and a non-natural base comprising nucleotide.

In one embodiment, the modified nucleotide comprises a short sequence of 3′-terminal deoxy-thymine nucleotides (dT).

In one embodiment, the modifications on the nucleotides are 2′-O-methyl, GNA and 2′fluoro modifications.

In some embodiments, the dsRNA agent further comprises at least one phosphorothioate internucleotide linkage.

In one embodiment, the dsRNA agent comprises 6-8 phosphorothioate internucleotide linkages.

In one embodiment, each strand is no more than 30 nucleotides in length.

In one embodiment, at least one strand comprises a 3′ overhang of at least 1 nucleotide. In another embodiment, at least one strand comprises a 3′ overhang of at least 2 nucleotides.

The double stranded region may be 15-30 nucleotide pairs in length; 17-23 nucleotide pairs in length; 17-25 nucleotide pairs in length; 23-27 nucleotide pairs in length; 19-21 nucleotide pairs in length; or 21-23 nucleotide pairs in length.

Each strand may have 19-30 nucleotides; 19-23 nucleotides; or 21-23 nucleotides.

In one embodiment, one or more lipophilic moieties are conjugated to one or more internal positions on at least one strand, such as via a linker or carrier.

In one embodiment, the internal positions include all positions except the terminal two positions from each end of the at least one strand.

In another embodiment, the internal positions include all positions except the terminal three positions from each end of the at least one strand.

In one embodiment, the internal positions exclude a cleavage site region of the sense strand.

In one embodiment, the internal positions include all positions except positions 9-12, counting from the 5′-end of the sense strand.

In another embodiment, the internal positions include all positions except positions 11-13, counting from the 3′-end of the sense strand.

In one embodiment, the internal positions exclude a cleavage site region of the antisense strand.

In one embodiment, the internal positions include all positions except positions 12-14, counting from the 5′-end of the antisense strand.

In one embodiment, the internal positions include all positions except positions 11-13 on the sense strand, counting from the 3′-end, and positions 12-14 on the antisense strand, counting from the 5′-end.

In one embodiment, the one or more lipophilic moieties are conjugated to one or more of the internal positions selected from the group consisting of positions 4-8 and 13-18 on the sense strand, and positions 6-10 and 15-18 on the antisense strand, counting from the 5′end of each strand.

In another embodiment, the one or more lipophilic moieties are conjugated to one or more of the internal positions selected from the group consisting of positions 5, 6, 7, 15, and 17 on the sense strand, and positions 15 and 17 on the antisense strand, counting from the 5′-end of each strand.

In one embodiment, the internal positions in the double stranded region exclude a cleavage site region of the sense strand.

In one embodiment, the sense strand is 21 nucleotides in length, the antisense strand is 23 nucleotides in length, and the lipophilic moiety is conjugated to position 21, position 20, position 15, position 1, position 7, position 6, or position 2 of the sense strand or position 16 of the antisense strand.

In one embodiment, the lipophilic moiety is conjugated to position 21, position 20, position 15, position 1, or position 7 of the sense strand.

In another embodiment, the lipophilic moiety is conjugated to position 21, position 20, or position 15 of the sense strand.

In yet another embodiment, the lipophilic moiety is conjugated to position 20 or position 15 of the sense strand.

In one embodiment, the lipophilic moiety is conjugated to position 16 of the antisense strand.

In one embodiment, the lipophilic moiety is an aliphatic, alicyclic, or polyalicyclic compound.

In one embodiment, the lipophilic moiety is selected from the group consisting of lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl) lithocholic acid, O3-(oleoyl)cholenic acid, dimethoxytrityl, or phenoxazine.

In one embodiment, the lipophilic moiety contains a saturated or unsaturated C4-C30 hydrocarbon chain, and an optional functional group selected from the group consisting of hydroxyl, amine, carboxylic acid, sulfonate, phosphate, thiol, azide, and alkyne.

In one embodiment, the lipophilic moiety contains a saturated or unsaturated C6-C18 hydrocarbon chain.

In one embodiment, the lipophilic moiety contains a saturated or unsaturated C16 hydrocarbon chain.

In one embodiment, the ligand is conjugated at the 2′-position of a nucleotide or modified nucleotide within the sense or antisense strand. For example, a C16 ligand may be conjugated as shown in the following structure:

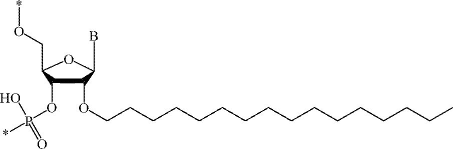

where * denotes a bond to an adjacent nucleotide, and B is a nucleobase or a nucleobase analog, optionally where B is adenine, guanine, cytosine, thymine or uracil.

In one embodiment, the saturated or unsaturated C16 hydrocarbon chain is conjugated to position 6, counting from the 5′-end of the strand.

In one embodiment, the lipophilic moiety is conjugated via a carrier that replaces one or more nucleotide(s) in the internal position(s) or the double stranded region.

In one embodiment, the carrier is a cyclic group selected from the group consisting of pyrrolidinyl, pyrazolinyl, pyrazolidinyl, imidazolinyl, imidazolidinyl, piperidinyl, piperazinyl, [1,3]dioxolanyl, oxazolidinyl, isoxazolidinyl, morpholinyl, thiazolidinyl, isothiazolidinyl, quinoxalinyl, pyridazinonyl, tetrahydrofuranyl, and decalinyl; or is an acyclic moiety based on a serinol backbone or a diethanolamine backbone.

In one embodiment, the lipophilic moiety is conjugated to the double-stranded iRNA agent via a linker containing an ether, thioether, urea, carbonate, amine, amide, maleimide-thioether, disulfide, phosphodiester, sulfonamide linkage, a product of a click reaction, or carbamate.

In one embodiment, the lipophilic moiety is conjugated to a nucleobase, sugar moiety, or internucleosidic linkage.

In one embodiment, the lipophilic moiety or targeting ligand is conjugated via a bio-cleavable linker selected from the group consisting of DNA, RNA, disulfide, amide, functionalized monosaccharides or oligosaccharides of galactosamine, glucosamine, glucose, galactose, mannose, and combinations thereof.

In one embodiment, the 3′ end of the sense strand is protected via an end cap which is a cyclic group having an amine, said cyclic group being selected from the group consisting of pyrrolidinyl, pyrazolinyl, pyrazolidinyl, imidazolinyl, imidazolidinyl, piperidinyl, piperazinyl, [1,3] dioxolanyl, oxazolidinyl, isoxazolidinyl, morpholinyl, thiazolidinyl, isothiazolidinyl, quinoxalinyl, pyridazinonyl, tetrahydrofuranyl, and decalinyl.

In one embodiment, the targeting ligand is a GalNAc conjugate.

In one embodiment, the dsRNA agent further comprises a terminal, chiral modification occurring at the first internucleotide linkage at the 3′ end of the antisense strand, having the linkage phosphorus atom in Sp configuration, a terminal, chiral modification occurring at the first internucleotide linkage at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp configuration or Sp configuration.

In another embodiment, the dsRNA agent further comprises a terminal, chiral modification occurring at the first and second internucleotide linkages at the 3′ end of the antisense strand, having the linkage phosphorus atom in Sp configuration, a terminal, chiral modification occurring at the first internucleotide linkage at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp or Sp configuration.

In yet another embodiment, the dsRNA agent further comprises a terminal, chiral modification occurring at the first, second and third internucleotide linkages at the 3′ end of the antisense strand, having the linkage phosphorus atom in Sp configuration, a terminal, chiral modification occurring at the first internucleotide linkage at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp or Sp configuration.

In another embodiment, the dsRNA agent further comprises a terminal, chiral modification occurring at the first, and second internucleotide linkages at the 3′ end of the antisense strand, having the linkage phosphorus atom in Sp configuration, a terminal, chiral modification occurring at the third internucleotide linkages at the 3′ end of the antisense strand, having the linkage phosphorus atom in Rp configuration, a terminal, chiral modification occurring at the first internucleotide linkage at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp or Sp configuration.

In another embodiment, the dsRNA agent further comprises a terminal, chiral modification occurring at the first, and second internucleotide linkages at the 3′ end of the antisense strand, having the linkage phosphorus atom in Sp configuration, a terminal, chiral modification occurring at the first, and second internucleotide linkages at the 5′ end of the antisense strand, having the linkage phosphorus atom in Rp configuration, and a terminal, chiral modification occurring at the first internucleotide linkage at the 5′ end of the sense strand, having the linkage phosphorus atom in either Rp or Sp configuration.

In one embodiment, the dsRNA agent further comprises a phosphate or phosphate mimic at the 5′-end of the antisense strand.

In one embodiment, the phosphate mimic is a 5′-vinyl phosphonate (VP).

In one embodiment, the base pair at the 1 position of the 5′-end of the antisense strand of the duplex is an AU base pair.

In one embodiment, the sense strand has a total of 21 nucleotides and the antisense strand has a total of 23 nucleotides.

The present invention also provides cells and pharmaceutical compositions for inhibiting expression of a gene encoding ALK comprising the dsRNA agents of the invention, such.

In one embodiment, the dsRNA agent is in an unbuffered solution, such as saline or water.

In another embodiment, the dsRNA agent is in a buffer solution, such as a buffer solution comprising acetate, citrate, prolamine, carbonate, or phosphate or any combination thereof; or phosphate buffered saline (PBS).

In one aspect, the present invention provides a method of inhibiting expression of an ALK gene in a cell, the method comprising contacting the cell with a dsRNA agent of the invention, or a pharmaceutical composition of the invention, thereby inhibiting expression of the ALK gene in the cell.

In one embodiment, cell is within a subject.

In one embodiment, the subject is a human.

In one embodiment, the subject has an ALK-associated disorder.

In one embodiment, the subject has type 2 diabetes.

In one embodiment, the subject has obesity.

In one embodiment, the subject is overweight.

In one embodiment, the subject is in need or desire of weight loss.

In one embodiment, the subject is in need or desire of weight maintenance.

In one embodiment, the subject has obesity-associated disorder.

In one embodiment, the obesity-associated disorder in the subject is selected from the group consisting of type 2 diabetes, hypertension, dyslipidemia, atherosclerosis, coronary heart disease, stroke, metabolic syndrome, gallbladder disease, fatty liver, osteoarthritis, sleep apnea, breathing problems, various types of cancer (e.g., endometrial cancer, esophageal adenocarcinoma, gastric cardia cancer, liver cancer, kidney cancer, pancreatic cancer), mental illness (e.g., depression, anxiety), body pain, and difficulty with physical functioning.

In one embodiment, contacting the cell with the dsRNA agent inhibits the expression of ALK by at least 30%.

In one embodiment, inhibiting expression of ALK decreases ALK protein level in serum of the subject by at least 30%.

In one aspect, the present invention provides method of treating a subject having a disorder that would benefit from reduction in ALK expression, comprising administering to the subject a therapeutically effective amount of a dsRNA agent of the invention, or a pharmaceutical composition of the invention, thereby treating the subject having the disorder that would benefit from reduction in ALK expression.

In another aspect, the present invention provides a method of preventing at least one symptom or sign in a subject having a disorder that would benefit from reduction in ALK expression, comprising administering to the subject a prophylactically effective amount of a dsRNA agent of the invention, or a pharmaceutical composition of the invention, thereby preventing at least one symptom or sign in the subject having the disorder that would benefit from reduction in ALK expression.

In another aspect, the present invention provides a method of preventing a subject from having a disorder that would benefit from reduction in ALK expression, comprising administering to the subject a prophylactically effective amount of the dsRNA agent of the invention, or the pharmaceutical composition of the invention, thereby preventing the subject from having the disorder that would benefit from reduction in ALK expression.

In one embodiment, the disorder is an ALK-associated disorder.

In one embodiment, the disorder is type 2 diabetes.

In one embodiment, the disorder is obesity.

In another embodiment, the disorder is an obesity-associated disorder.

In one embodiment, the obesity-associated disorder is selected from the group consisting of type 2 diabetes, hypertension, dyslipidemia, atherosclerosis, coronary heart disease, stroke, metabolic syndrome, gallbladder disease, fatty liver, osteoarthritis, sleep apnea, breathing problems, various types of cancer (e.g., endometrial cancer, esophageal adenocarcinoma, gastric cardia cancer, liver cancer, kidney cancer, pancreatic cancer), mental illness (e.g., depression, anxiety), body pain, and difficulty with physical functioning. In one embodiment, the subject is human.

In one embodiment, the administration of the agent to the subject causes a decrease in body weight.

In one embodiment, the administration of the agent to the subject causes a decrease in waist circumference.

In one embodiment, the administration of the agent to the subject causes a decrease in hip circumference.

In one embodiment, the administration of the agent to the subject causes a decrease in fat deposition.

In one embodiment, the administration of the agent to the subject causes a decrease in triglyceride levels.

In one embodiment, the administration of the agent to the subject causes an improvement in blood lipid profile.

In one embodiment, the administration of the agent to the subject causes an improvement in blood glucose levels.

In one embodiment, the dsRNA agent is administered to the subject at a dose of about 0.01 mg/kg to about 50 mg/kg.

In one embodiment, the dsRNA agent is administered to the subject subcutaneously.

In another embodiment, the dsRNA agent is administered to the subject intrathecally.

In one embodiment, the methods of the invention further comprise determining the level of ALK in a sample(s) from the subject.

In one embodiment, the level of ALK in the subject sample(s) is an ALK protein level in a blood, serum, or cerebrospinal fluid sample(s).

In one embodiment, the methods of the invention further comprise administering to the subject an additional therapeutic agent.

In one aspect, the present invention provides a kit comprising a dsRNA agent of the invention, or a pharmaceutical composition of the invention.

In another aspect, the present invention provides a vial comprising a dsRNA agent of the invention, or a pharmaceutical composition of the invention.

In yet another aspect, the present invention provides a syringe comprising a dsRNA agent of the invention, or a pharmaceutical composition of the invention.

In another aspect, the present invention provides an intrathecal pump comprising a dsRNA agent of the invention, or a pharmaceutical composition of the invention.

### DETAILED DESCRIPTION OF THE INVENTION

The present disclosure provides RNAi compositions, which effect the RNA-induced silencing complex (RISC)-mediated cleavage of RNA transcripts of an ALK gene. The ALK gene may be within a cell, e.g., a cell within a subject, such as a human. The use of these iRNAs enables the targeted degradation of mRNAs of the corresponding gene (ALK gene) in mammals.

The iRNAs of the invention have been designed to target an ALK gene, e.g., an ALK gene either with or without nucleotide modifications. The iRNAs of the invention inhibit the expression of the ALK gene by at least about 30%, 35%, 40%, 45%, 50%, 55%, 60%, 65%, 70%, 75%, 80%, 85%, 90%, 95%, or 99%. Without intending to be limited by theory, it is believed that a combination or sub-combination of the foregoing properties and the specific target sites, or the specific modifications in these iRNAs confer to the iRNAs of the invention improved efficacy, stability, potency, durability, and safety.

Accordingly, the present disclosure also provides methods of using the RNAi compositions of the disclosure for inhibiting the expression of an ALK gene or for treating a subject having a disorder that would benefit from inhibiting or reducing the expression of an ALK gene, e.g., type 2 diabetes, obesity, and obesity-associated disease, e.g., coronary heart disease, and for supporting weight loss or weight maintenance in a subject.

The RNAi agents of the disclosure include an RNA strand (the antisense strand) having a region which is about 30 nucleotides or less in length, e.g., 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length, which region is substantially complementary to at least part of an mRNA transcript of an ALK gene, e.g., an ALK exon. In certain embodiments, the RNAi agents of the disc¹ )l^{N}^{A} strand (the antisense strand) having a region which is about 21-23 nucleotides in length, which region is substantially complementary to at least part of an mRNA transcript of an ALK gene.

In certain embodiments, the RNAi agents of the disclosure include an RNA strand (the antisense strand) which can include longer lengths, for example up to 66 nucleotides, e.g., 36-66, 26-36, 25-36, 31-60, 22-43, 27-53 nucleotides in length with a region of at least 19 contiguous nucleotides that is substantially complementary to at least a part of an mRNA transcript of an ALK gene. These RNAi agents with the longer length antisense strands preferably include a second RNA strand (the sense strand) of 20-60 nucleotides in length wherein the sense and antisense strands form a duplex of 18-30 contiguous nucleotides.

The use of these RNAi agents enables the targeted degradation and/or inhibition of mRNAs of an ALK gene in mammals. Thus, methods and compositions including these RNAi agents are useful for treating a subject who would benefit by a reduction in the levels or activity of an ALK protein, such as a subject having type 2 diabetes, obesity or an obesity-associated disease, such as coronary heart disease.

The following detailed description discloses how to make and use compositions containing RNAi agents to inhibit the expression of an ALK gene, as well as compositions and methods for treating subjects having diseases and disorders that would benefit from inhibition or reduction of the expression of the genes.

### I. Definitions

In order that the present disclosure may be more readily understood, certain terms are first defined. In addition, it should be noted that whenever a value or range of values of a parameter are recited, it is intended that values and ranges intermediate to the recited values are also intended to be part of this disclosure.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”. The term “or” is used herein to mean, and is used interchangeably with, the term “and/or,” unless context clearly indicates otherwise.

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the nr^{r}^{w}_{’”}⁻ nucleic acid molecule must be an integer. For example, “at least 18 nucleotides of a 21 nucleotide nucleic acid molecule” means that 18, 19, 20, or 21 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range.

As used herein, methods of detection can include determination that the amount of analyte present is below the level of detection of the method.

In the event of a conflict between an indicated target site and the nucleotide sequence for a sense or antisense strand, the indicated sequence takes precedence.

In the event of a conflict between a chemical structure and a chemical name, the chemical structure takes precedence.

As used herein, the term “anaplastic lymphoma kinase” used interchangeably with the term “ALK,” refers to the well-known gene and polypeptide encoded by that gene, also known in the art as “ALK tyrosine kinase receptor,” “ALK receptor tyrosine kinase,” “anaplastic lymphoma receptor tyrosine kinase,” “NBLST3,” “cluster of differentiation 246,” and “CD246”. The ALK gene is active in the brain and other tissues throughout the body. ALK is expressed in various regions of the central nervous system, including the hypothalamus, cerebellum, cerebral cortex, pituitary gland (the central endocrine gland that controls other endocrine glands); endocrine tissues including the thyroid gland and the adrenal gland; and throughout the body.

ALK encodes a receptor tyrosine kinase, which belongs to the insulin receptor superfamily. The ALK protein comprises an extracellular domain, a hydrophobic stretch corresponding to a single pass transmembrane region, and an intracellular kinase domain. It plays an important role in the development of the brain. The ALK gene has been found to be rearranged, mutated, or amplified in a series of tumors including anaplastic large cell lymphomas, neuroblastoma, and non-small cell lung cancer. The chromosomal rearrangements result in creation of multiple fusion genes in tumorigenesis.

Genome-wide association studies and gene data analysis revealed that several variants within the ALK gene are associated with thinness or body mass index (BMI) in the Estonian Biobank (Estonian Genome Center of the University of Tartu; EGCUT), the UK Biobank (UKBB), and the GIANT consortium database. Prior studies also demonstrated that ALK variants are associated with different metabolic traits such as adiponectin levels, lipid glucose levels, and lipid homeostasis. (Li et al., 2015, Sci. Rep. 5: 13422; Palmer et al., 2015, Diabetes 64: 1853-66; Lettre et al., 2011, PLoS Genet. 7:e1001300; Kathiresan et al., 2009, Nat. Genet. 41:56-65; Meigs et al., 2007, BMC Med. Genet. 8(Supple 1):S16).

Genetic deletion of ALK in mice resulted in thin animals on normal diet, and resistance to obesity on high-fat diet or under leptin deficiency. ALK knockout mice also showed increased energy expenditure and improved glucose tolerance. Site-specific knockdown of ALK in the paraventricular nucleus (PVN) in the hypothalamus was sufficient to induce resistance to high fat diet-induced obesity and reduced feeding efficiency in mice. ALK deficient mice are viable, fertile, and show no obvious phenotypes except for thinness and resistance to obesogenic diets. In drosophila, downregulation of ALK using RNAi resulted in reduced triglyceride levels. (Orthofer et al., 2020, Cell181, https://doi.org/10.1016/j.cell.2020.04.034). The improved glucose tolerance in ALK knockout mice supports the use of the agents or methods of the present invention to treat or prevent type 2 diabetes.

Mechanistic studies in mice revealed that reduced ALK expression in the hypothalamus increased systemic energy expenditure by altering sympathetic control of the adipose tissue and facilitating lipolysis. (Orthofer et al., 2020, Cell 181, https://doi.org/10.1016/j.cell.2020.04.034).

Leptin is a hormone produced by the adipose tissue and acts on its receptors the brain, stimulating white fat breakdown through the action of sympathetic nerve fibers that innervate the adipose tissue. (Zeng et al, 2015, Cell 163:1;84-94) The finding that ALK inactivation results in resistance to obesity in leptin-deficient mice suggests that ALK inhibition may promote thinness even in leptin resistance and other conditions of general and morbid obesity. (Orthofer et al., 2020, Cell 181, https://doi.org/10.1016/j.cell.2020.04.034).

Exemplary nucleotide and amino acid sequences of ALK can be found, for example, at GenBank Accession No. NM_004304.4 (Homo sapiens ALK, SEQ ID NO: 1, reverse complement, SEQ ID NO: 2); GenBank Accession No.: XM_005576218.2 (Macaca fascicularis ALK, SEQ ID NO: 3, reverse complement, SEQ ID NO: 4); GenBank Accession No. NM_007439.2 (Mus musculus ALK, SEQ ID NO: 5; reverse complement, SEQ ID NO: 6); and GenBank Accession No.: NM_001169101.2 (Rattus norvegicus ALK, SEQ ID NO: 7, reverse complement, SEQ ID NO: 8).

The nucleotide sequence of the genomic region of human chromosome harboring the ALK gene may be found in, for example, the Genome Reference Consortium Human Build 38 (also referred to as Human Genome build 38 or GRCh38) available at GenBank. The nucleotide sequence of the genomic region of human chromosome 2 harboring the ALK gene may also be found at, for example, GenBank Accession No. NC_000002.12, corresponding to nucleotides 29190992 - 29921589 of human chromosome 2. The nucleotide sequence of the human ALK gene may be found in, for example, GenBank Accession No. NG_009445.1.

Further examples of ALK sequences can be found in publically available databases, for example, GenBank, OMIM, and UniProt.

Additional information on ALK can be found, for example, at https://www.ncbi.nlm.nih.gov/gene/238.The term ALK as used herein also refers to variations of the ALK gene including variants provided in the clinical variant database, for example, at https://www.ncbi.nlm.nih.gov/clinvar/?term=NM_004304.4.

The entire contents of each of the foregoing GenBank Accession numbers and the Gene database numbers are incorporated herein by reference as of the date of filing this application.

As used herein, “target sequence” refers to a contiguous portion of the nucleotide sequence of an mRNA molecule formed during the transcription of an ALK gene, including mRNA that is a product of RNA processing of a primary transcription product. In one embodiment, the target portion of the sequence will be at least long enough to serve as a substrate for RNAi-directed cleavage at or near that portion of the nucleotide sequence of an mRNA molecule formed during the transcription of an ALK gene.

The target sequence is about 15-30 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length. In certain embodiments, the target sequence is 19-23 nucleotides in length, optionally 21-23 nucleotides in length. Ranges and lengths intermediate to the above recited ranges and lengths are also contemplated to be part of the disclosure.

As used herein, the term “strand comprising a sequence” refers to an oligonucleotide comprising a chain of nucleotides that is described by the sequence referred to using the standard nucleotide nomenclature. “G,” “C,” “A,” “T”, and “U” each generally stand for a nucleotide that contains guanine, cytosine, adenine, thymidine, and uracil as a base, respectively in the context of a modified or unmodified nucleotide. However, it will be understood that the term “ribonucleotide” or “nucleotide” can also refer to a modified nucleotide, as further detailed below, or a surrogate replacement moiety (see, e.g., Table 1). The skilled person is well aware that guanine, cytosine, adenine, thymidine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the disclosure by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the disclosure.

The terms “iRNA”, “RNAi agent,” “iRNA agent,” “RNA interference agent” as used interchangeably herein, refer to an agent that contains RNA as that term is defined herein, and which mediates the targeted cleavage of an RNA transcript via an RNA-induced silencing complex (RISC) pathway. RNA interference (RNAi) is a process that directs the sequence-specific degradation of mRNA. RNAi modulates, e.g., inhibits, the expression of ALK in a cell, e.g., a cell within a subject, such as a mammalian subject.

In one embodiment, an RNAi agent of the disclosure includes a single stranded RNAi that interacts with a target RNA sequence, e.g., an ALK target mRNA sequence, to direct the cleavage of the target RNA. Without wishing to be bound by theory it is believed that long double stranded RNA introduced into cells is broken down into double-stranded short interfering RNAs (siRNAs) comprising a sense strand and an antisense strand by a Type III endonuclease known as Dicer (Sharp et al. (2001) Genes Dev. 15:485). Dicer, a ribonuclease-III-like enzyme, processes this dsRNA into 19-23 base pair short interfering RNAs with characteristic two base 3′ overhangs (Bernstein, et al., (2001) Nature 409:363). These siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15: 188). Thus, in one aspect the disclosure relates to a single stranded RNA (ssRNA) (the antisense strand of a siRNA duplex) generated within a cell and which promotes the formation of a RISC complex to effect silencing of the target gene, i.e., an ALK gene. Accordingly, the term “siRNA” is also used herein to refer to an RNAi as described above.

In another embodiment, the RNAi agent may be a single-stranded RNA that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded RNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In another embodiment, a “RNAi agent” for use in the compositions and methods of the disclosure is a double stranded RNA and is referred to herein as a “double stranded RNAi agent,” “double stranded RNA (dsRNA) molecule,” “dsRNA agent,” or “dsRNA”. The term “dsRNA” refers to a complex of ribonucleic acid molecules, having a duplex structure comprising two anti-parallel and substantially complementary nucleic acid strands, referred to as having “sense” and “antisense” orientations with respect to a target RNA, i.e., an ALK gene. In some embodiments of the disclosure, a double stranded RNA (dsRNA) triggers the degradation of a target RNA, e.g., an mRNA, through a post-transcriptional gene-silencing mechanism referred to herein as RNA interference or RNAi.

In general, a dsRNA molecule can include ribonucleotides, but as described in detail herein, each or both strands can also include one or more non-ribonucleotides, e.g., a deoxyribonucleotide, a modified nucleotide. In addition, as used in this specification, an “RNAi agent” may include ribonucleotides with chemical modifications; an RNAi agent may include substantial modifications at multiple nucleotides. As used herein, the term “modified nucleotide” refers to a nucleotide having, independently, a modified sugar moiety, a modified internucleotide linkage, or a modified nucleobase. Thus, the term modified nucleotide encompasses substitutions, additions or removal of, e.g., a functional group or atom, to internucleoside linkages, sugar moieties, or nucleobases. The modifications suitable for use in the agents of the disclosure include all types of modifications disclosed herein or known in the art. Any such modifications, as used in a siRNA type molecule, are encompassed by “RNAi agent” for the purposes of this specification and claims.

In certain embodiments of the instant disclosure, inclusion of a deoxy-nucleotide if present within an RNAi agent can be considered to constitute a modified nucleotide.

The duplex region may be of any length that permits specific degradation of a desired target RNA through a RISC pathway, and may range from about 15-36 base pairs in length, for example, about 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. In certain embodiments, the duplex region is 19-21 base pairs in length, e.g., 21 base pairs in length. Ranges and lengths intermediate to the above recited ranges and lengths are also contemplated to be part of the disclosure.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at least 4, at least 5, at least 6, at least 7, at least 8, at least 9, at least 10, at least 20, at least 23 or more unpaired nucleotides or nucleotides not directed to the target site of the dsRNA. In some embodiments, the hairpin loop can be 10 or fewer nucleotides. In some embodiments, the hairpin loop can be 8 or fewer unpaired nucleotides. In some embodiments, the hairpin loop can be 4-10 unpaired nucleotides. In some embodiments, the hairpin loop can be 4-8 nucleotides.

Where the two substantially complementary strands of a dsRNA are comprised by separate RNA molecules, those molecules need not, but can be covalently connected. In certain embodiments where the two strands are connected covalently by means other than an uninterrupted chain of nucleotides between the 3′-end of one strand and the 5′-end of the respective other strand forming the duplex structure, the connecting structure is referred to as a “linker” (though it is noted that certain other structures defined elsewhere herein can also be referred to as a “linker”). The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs. In one embodiment of the RNAi agent, at least one strand comprises a 3′ overhang of at least 1 nucleotide. In another embodiment, at least one strand comprises a 3′ overhang of at least 2 nucleotides, e.g., 2, 3, 4, 5, 6, 7, 9, 10, 11, 12, 13, 14, or 15 nucleotides. In other embodiments, at least one strand of the RNAi agent comprises a 5′ overhang of at least 1 nucleotide. In certain embodiments, at least one strand comprises a 5′ overhang of at least 2 nucleotides, e.g., 2, 3, 4, 5, 6, 7, 9, 10, 11, 12, 13, 14, or 15 nucleotides. In still other embodiments, both the 3′ and the 5′ end of one strand of the RNAi agent comprise an overhang of at least 1 nucleotide.

In one embodiment, an RNAi agent of the disclosure is a dsRNA, each strand of which independently comprises 19-23 nucleotides, that interacts with a target RNA sequence, e.g., an ALK target mRNA sequence, to direct the cleavage of the target RNA.

In some embodiments, an iRNA of the invention is a dsRNA of 24-30 nucleotides that interacts with a target RNA sequence, e.g., an ALK target mRNA sequence, to direct the cleavage of the target RNA.

As used herein, the term “nucleotide overhang” refers to at least one unpaired nucleotide that protrudes from the duplex structure of an RNAi agent, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively, the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

In one embodiment, the antisense strand of a dsRNA has a 1-10 nucleotide, e.g., a 1, 2, 3, 4, 5, 6, 7, 8, 9, or 10 nucleotide, overhang at the 3′-end or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the antisense strand of a dsRNA has a 1-10 nucleotide, e.g., 0-3, 1-3, 2-4, 2-5, 4-10, 5-10, e.g., a 1, 2, 3, 4, 5, 6, 7, 8, 9, or 10 nucleotide, overhang at the 3′-end or the 5′-end. In one embodiment, the sense strand of a dsRNA has a 1-10 nucleotide, e.g., a 1, 2, 3, 4, 5, 6, 7, 8, 9, or 10 nucleotide, overhang at the 3′-end or the 5′-end. In another embodiment, one or more of the nucleotides in the overhang is replaced with a nucleoside thiophosphate.

In certain embodiments, the antisense strand of a dsRNA has a 1-15 nucleotide, e.g., 0-3, 1-3, 2-4, 2-5, 4-10, 5-10, 6-12 or e.g., a 1, 2, 3, 4, 5, 6, 7, 8, 9, 10, 11, 12, 13, 14, or 15 nucleotide, overhang at the 3′-end. In another embodiment, one or more of the nucleotides in the overhang is replaced with a nucleoside thiophosphate.

In certain embodiments, the overhang on the sense strand or the antisense strand, can include extended lengths longer than 10 or 15 nucleotides, e.g., 1-30 nucleotides, 2-30 nucleotides, 10-30 nucleotides, or 10-15 nucleotides in length. In certain embodiments, an extended overhang is on the sense strand of the duplex. In certain embodiments, an extended overhang is present on the 3′end of the sense strand of the duplex. In certain embodiments, an extended overhang is present on the 5′end of the sense strand of the duplex. In certain embodiments, an extended overhang is on the antisense strand of the duplex. In certain embodiments, an extended overhang is present on the 3′end of the antisense strand of the duplex. In certain embodiments, an extended overhang is present on the 5′end of the antisense strand of the duplex. In certain embodiments, one or more of the nucleotides in the overhang is replaced with a nucleoside thiophosphate. In certain embodiments, the overhang includes a self-complementary portion such that the overhang is capable of forming a hairpin structure that is stable under physiological conditions.

The terms “blunt” or “blunt ended” as used herein in reference to a dsRNA mean that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double stranded over its entire length.

The term “antisense strand” or “guide strand” refers to the strand of an RNAi agent, e.g., a dsRNA, which includes a region that is substantially complementary to a target sequence, e.g., an ALK mRNA.

As used herein, the term “region of complementarity” refers to the region on the antisense strand that is substantially complementary to a sequence, for example a target sequence, e.g., an ALK nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′- or 3′-terminus of the RNAi agent. In some embodiments, a double stranded RNA agent of the invention includes a nucleotide mismatch in the antisense strand. In some embodiments, the antisense strand of the double stranded RNA agent of the invention includes no more than 4 mismatches with the target mRNA, e.g., the antisense strand includes 4, 3, 2, 1, or 0 mismatches with the target mRNA. In some embodiments, the antisense strand double stranded RNA agent of the invention includes no more than 4 mismatches with the sense strand, e.g., the antisense strand includes 4, 3, 2, 1, or 0 mismatches with the sense strand. In some embodiments, a double stranded RNA agent of the invention includes a nucleotide mismatch in the sense strand. In some embodiments, the sense strand of the double stranded RNA agent of the invention includes no more than 4 mismatches with the antisense strand, e.g., the sense strand includes 4, 3, 2, 1, or 0 mismatches with the antisense strand. In some embodiments, the nucleotide mismatch is, for example, within 5, 4, 3 nucleotides from the 3′-end of the iRNA. In another embodiment, the nucleotide mismatch is, for example, in the 3′-terminal nucleotide of the iRNA agent. In some embodiments, the mismatch(s) is not in the seed region.

Thus, an RNAi agent as described herein can contain one or more mismatches to the target sequence. In one embodiment, an RNAi agent as described herein contains no more than 3 mismatches (i.e., 3, 2, 1, or 0 mismatches). In one embodiment, an RNAi agent as described herein contains no more than 2 mismatches. In one embodiment, an RNAi agent as described herein contains no more than 1 mismatch. In one embodiment, an RNAi agent as described herein contains 0 mismatches. In certain embodiments, if the antisense strand of the RNAi agent contains mismatches to the target sequence, the mismatch can optionally be restricted to be within the last 5 nucleotides from either the 5′ - or 3′-end of the region of complementarity. For example, in such embodiments, for a 23 nucleotide RNAi agent, the strand which is complementary to a region of an ALK gene, generally does not contain any mismatch within the central 13 nucleotides. The methods described herein or methods known in the art can be used to determine whether an RNAi agent containing a mismatch to a target sequence is effective in inhibiting the expression of an ALK gene. Consideration of the efficacy of RNAi agents with mismatches in inhibiting expression of an ALK gene is important, especially if the particular region of complementarity in an ALK gene is known to have polymorphic sequence variation within the population.

As used herein, “substantially all of the nucleotides are modified” are largely but not wholly modified and can include not more than 5, 4, 3, 2, or 1 unmodified nucleotides.

The term “sense strand” or “passenger strand” as used herein, refers to the strand of an RNAi agent that includes a region that is substantially complementary to a region of the antisense strand as that term is defined herein.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an RNAi agent, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogsteen base pairing.

The terms “complementary,” “fully complementary” and “substantially complementary” herein can be used with respect to the base matching between the sense strand and the antisense strand of a dsRNA, or between the antisense strand of an RNAi agent and a target sequence, as will be understood from the context of their use.

As used herein, a polynucleotide that is “substantially complementary to at least part of′ a messenger RNA (mRNA) refers to a polynucleotide that is substantially complementary to a contiguous portion of the mRNA of interest (e.g., an mRNA encoding ALK). For example, a polynucleotide is complementary to at least a part of an ALK mRNA if the sequence is substantially complementary to a non-interrupted portion of an mRNA encoding ALK.

Accordingly, in some embodiments, the antisense polynucleotides disclosed herein are fully complementary to the target ALK sequence. In other embodiments, the antisense polynucleotides disclosed herein are substantially complementary to the target ALK sequence and comprise a contiguous nucleotide sequence which is at least 80% complementary over its entire length to the equivalent region of the nucleotide sequence of any one of SEQ ID NOs: 1, 3, 5 and 7, or a fragment of any one of SEQ ID NOs: 1, 3, 5 and 7, such as about 85%, about 90%, about 91%, about 92%, about 93%, about 94%, about 95%, about 96%, about 97%, about 98%, or about 99% complementary.

In some embodiments, the antisense polynucleotides disclosed herein are substantially complementary to a fragment of a target ALK sequence and comprise a contiguous nucleotide sequence which is at least 80% complementary over its entire length to a fragment of SEQ ID NO: 1 selected from the group of nucleotides 22-44, 55-77, 155-177, 243-265, 284-306, 312-334, 324-346, 341-363, 364-386, 380-402, 428-450, 446-468, 463-485, 497-519, 510-532, 585-607, 597-619, 610-632, 622-644, 634-656, 686-708, 700-722, 712-734, 752-774, 772-794, 796-818, 811-833, 825-847, 845-867, 857-879, 871-893, 917-939, 953-975, 985-1007, 1077-1099, 1098-1120, 1110-1132, 1122-1144, 1134-1156, 1146-1168, 1158-1180, 1181-1203, 1316-1338, 1359-1381, 1371-1393, 1392-1414, 1443-1465, 1460-1482, 1475-1497, 1497-1519, 1509-1531, 1522-1544, 1544-1566, 1558-1580, 1592-1614, 1614-1636, 1627-1649, 1642-1664, 1657-1679, 1670-1692, 1682-1704, 1695-1717, 1707-1729, 1722-1744, 1742-1764, 1785-1807, 1799-1821, 1845-1867, 1877-1899, 1893-1915, 1905-1927, 1928-1950, 1940-1962, 1952-1974, 1970-1992, 1983-2005, 1998-2020, 2021-2043, 2061-2083, 2073-2095, 2092-2114, 2105-2127, 2117-2139, 2136-2158, 2150-2172, 2162-2184, 2174-2196, 2194-2216, 2213-2235, 2244-2266, 2267-2289, 2289-2311, 2312-2334, 2325-2347, 2337-2359, 2352-2374, 2364-2386, 2388-2410, 2400-2422, 2418-2440, 2442-2464, 2462-2484, 2475-2497, 2487-2509, 2501-2523, 2532-2554, 2554-2576, 2573-2595, 2585-2607, 2597-2619, 2610-2632, 2635-2657, 2649-2671, 2670-2692, 2682-2704, 2705-2727, 2718-2740, 2730-2752, 2751-2773, 2771-2793, 2804-2826, 2817-2839, 2829-2851, 2841-2863, 2853-2875, 2873-2895, 2887-2909, 2899-2921, 2912-2934, 2927-2949, 2952-2974, 2978-3000, 2990-3012, 3045-3067, 3062-3084, 3074-3096, 3116-3138, 3128-3150, 3140-3162, 3157-3179, 3177-3199, 3189-3211, 3203-3225, 3216-3238, 3231-3253, 3249-3271, 3261-3283, 3273-3295, 3303-3325, 3315-3337, 3330-3352, 3345-3367, 3363-3385, 3376-3398, 3422-3444, 3434-3456, 3462-3484, 3502-3524, 3524-3546, 3536-3558, 3554-3576, 3568-3590, 3583-3605, 3597-3619, 3609-3631, 3623-3645, 3637-3659, 3649-3671, 3749-3771, 3772-3794, 3816-3838, 3832-3854, 3853-3875, 3893-3915, 3909-3931, 3924-3946, 3944-3966, 3957-3979, 3969-3991, 3989-4011, 4005-4027, 4035-4057, 4050-4072, 4062-4084, 4095-4117, 4107-4129, 4125-4147, 4137-4159, 4149-4171, 4161-4183, 4176-4198, 4196-4218, 4209-4231, 4232-4254, 4244-4266, 4257-4279, 4273-4295, 4335-4357, 4352-4374, 4392-4414, 4414-4436, 4427-4449, 4440-4462, 4457-4479, 4472-4494, 4524-4546, 4556-4578, 4617-4639, 4640-4662, 4654-4676, 4669-4691, 4691-4713, 4703-4725, 4715-4737, 4732-4754, 4745-4767, 4760-4782, 4772-4794, 4790-4812, 4805-4827, 4818-4840, 4845-4867, 4857-4879, 4869-4891, 4884-4906, 4899-4921, 4911-4933, 4946-4968, 4958-4980, 4970-4992, 4998-5020, 5010-5032, 5022-5044, 5044-5066, 5056-5078, 5073-5095, 5090-5112, 5109-5131, 5121-5143, 5133-5155, 5145-5167, 5157-5179, 5171-5193, 5188-5210, 5214-5236, 5227-5249, 5279-5301, 5291-5313, 5305-5327, 5325-5347, 5337-5359, 5376-5398, 5390-5412, 5403-5425, 5426-5448, 5447-5469, 5459-5481, 5475-5497, 5507-5529, 5521-5543, 5557-5579, 5574-5596, 5612-5634, 5631-5653, 5643-5665, 5655-5677, 5669-5691, 5684-5706, 5703-5725, 5721-5743, 5747-5769, 5759-5781, 5771-5793, 5785-5807, 5812-5834, 5824-5846, 5843-5865, 5855-5877, 5867-5889, 5879-5901, 5895-5917, 5907-5929, 5922-5944, 5954-5976, 5970-5992, 5982-6004, 5995-6017, 6030-6052, 6042-6064, 6054-6076, 6077-6099, 6095-6117, 6108-6130, 6126-6148, 6138-6160, 6151-6173, 6181-6203, 6201-6223, 6232-6254, and 6245-6267 of SEQ ID NO: 1, such as about 85%, about 90%, about 91%, about 92%, about 93%, about 94%, about 95%, about 96%, about 97%, about 98%, or about 99% complementary. Ranges intermediate to the above recited ranges are also contemplated to be part of the disclosure.

In other embodiments, the antisense polynucleotides disclosed herein are substantially complementary to the target ALK sequence and comprise a contiguous nucleotide sequence which is at least about 80% complementary over its entire length to any one of the sense strand nucleotide sequences in Tables 2, 3, or 4, or a fragment of any one of the sense strand nucleotide sequences in Tables 2, 3, or 4, such as about 85%, about 90%, about 91%, about 92%, about 93%, about 94%, about 95%, about 96%, about 97%, about 98%, about 99%, or 100% complementary.

In one embodiment, an RNAi agent of the disclosure includes a sense strand that is substantially complementary to an antisense polynucleotide which, in turn, is the same as a target ALK sequence, and wherein the sense strand polynucleotide comprises a contiguous nucleotide sequence which is at least about 80% complementary over its entire length to the equivalent region of the nucleotide sequence of SEQ ID NOs: 1, 3, 5 and 7, or a fragment of any one of SEQ ID NOs: 1, 3, 5 and 7, such as about 85%, about 90%, about 91%, about 92%, about 93%, about 94%, about 95%, about 96%, about 97%, about 98%, about 99%, or 100% complementary.

In some embodiments, an iRNA of the invention includes a sense strand that is substantially complementary to an antisense polynucleotide which, in turn, is complementary to a target ALK sequence, and wherein the sense strand polynucleotide comprises a contiguous nucleotide sequence which is at least about 80% complementary over its entire length to any one of the antisense strand nucleotide sequences in Tables 2, 3, or 4, or a fragment of any one of the antisense strand nucleotide sequences in Tables 2, 3, or 4, such as about 85%, about 90%, about 91%, about 92%, about 93%, about 94%, about 95%, about 96%, about 97%, about 98%, about 99%, or 100% complementary.

In certain embodiments, the sense and antisense strands are selected from any one of duplexes AD-1289971, AD-1289972, AD-1289973, AD-1289974, AD-1289975, AD-1289976, AD-1289977, AD-1289978, AD-1289979, AD-1289980, AD-1289981, AD-1289982, AD-1289983, AD-1289984, AD-1289985, AD-1289986, AD-1289987, AD-1289988, AD-1289989, AD-1289990, AD-1289991, AD-1289992, AD-1289993, AD-1289994, AD-1289995, AD-1289996, AD-1289997, AD-1289998, AD-1289999, AD-1290000, AD-1290001, AD-1290002, AD-1290003, AD-1290004, AD-1290005, AD-1290006, AD-1290007, AD-1290008, AD-1290009, AD-1290010, AD-1290011, AD-1290012, AD-1290013, AD-1290014, AD-1290015, AD-1290016, AD-1290017, AD-1290018, AD-1290019, AD-1290020, AD-1290021, AD-1290022, AD-1290023, AD-1290024, AD-1290025, AD-1290026, AD-1290027, AD-1290028, AD-1290029, AD-1290030, AD-1290031, AD-1290032, AD-1290033, AD-1290034, AD-1290035, AD-1290036, AD-1290037, AD-1290038, AD-1290039, AD-1290040, AD-1290041, AD-1290042, AD-1290043, AD-1290044, AD-1290045, AD-1290046, AD-1290047, AD-1290048, AD-1290049, AD-1290050, AD-1290051, AD-1290052, AD-1290053, AD-1290054, AD-1290055, AD-1290056, AD-1290057, AD-1290058, AD-1290059, AD-1290060, AD-1290061, AD-1290062, AD-1290063, AD-1290064, AD-1290065, AD-1290066, AD-1290067, AD-1290068, AD-1290069, AD-1290070, AD-1290071, AD-1290072, AD-1290073, AD-1290074, AD-1290075, AD-1290076, AD-1290077, AD-1290078, AD-1290079, AD-1290080, AD-1290081, AD-1290082, AD-1290083, AD-1290084, AD-1290085, AD-1290086, AD-1290087, AD-1290088, AD-1290089, AD-1290090, AD-1290091, AD-1290092, AD-1290093, AD-1290094, AD-1290095, AD-1290096, AD-1290097, AD-1290098, AD-1290099, AD-1290100, AD-1290101, AD-1290102, AD-1290103, AD-1290104, AD-1290105, AD-1290106, AD-1290107, AD-1290108, AD-1290109, AD-1290110, AD-1290111, AD-1290112, AD-1290113, AD-1290114, AD-1290115, AD-1290116, AD-1290117, AD-1290118, AD-1290119, AD-1290120, AD-1290121, AD-1290122, AD-1290123, AD-1290124, AD-1290125, AD-1290126, AD-1290127, AD-1290128, AD-1290129, AD-1290130, AD-1290131, AD-1290132, AD-1290133, AD-1290134, AD-1290135, AD-1290136, AD-1290137, AD-1290138, AD-1290139, AD-1290140, AD-1290141, AD-1290142, AD-1290143, AD-1290144, AD-1290145, AD-1290146, AD-1290147, AD-1290148, AD-1290149, AD-1290150, AD-1290151, AD-1290152, AD-1290153, AD-1290154, AD-1290155, AD-1290156, AD-1290157, AD-1290158, AD-1290159, AD-1290160, AD-1290161, AD-1290162, AD-1290163, AD-1290164, AD-1290165, AD-1290166, AD-1290167, AD-1290168, AD-1290169, AD-1290170, AD-1290171, AD-1290172, AD-1290173, AD-1290174, AD-1290175, AD-1290176, AD-1290177, AD-1290178, AD-1290179, AD-1290180, AD-1290181, AD-1290182, AD-1290183, AD-1290184, AD-1290185, AD-1290186, AD-1290187, AD-1290188, AD-1290189, AD-1290190, AD-1290191, AD-1290192, AD-1290193, AD-1290194, AD-1290195, AD-1290196, AD-1290197, AD-1290198, AD-1290199, AD-1290200, AD-1290201, AD-1290202, AD-1290203, AD-1290204, AD-1290205, AD-1290206, AD-1290207, AD-1290208, AD-1290209, AD-1290210, AD-1290211, AD-1290212, AD-1290213, AD-1290214, AD-1290215, AD-1290216, AD-1290217, AD-1290218, AD-1290219, AD-1290220, AD-1290221, AD-1290222, AD-1290223, AD-1290224, AD-1290225, AD-1290226, AD-1290227, AD-1290228, AD-1290229, AD-1290230, AD-1290231, AD-1290232, AD-1290233, AD-1290234, AD-1290235, AD-1290236, AD-1290237, AD-1290238, AD-1290239, AD-1290240, AD-1290241, AD-1290242, AD-1290243, AD-1290244, AD-1290245, AD-1290246, AD-1290247, AD-1290248, AD-1290249, AD-1290250, AD-1290251, AD-1290252, AD-1290253, AD-1290254, AD-1290255, AD-1290256, AD-1290257, AD-1290258, AD-1290259, AD-1290260, AD-1290261, AD-1290262, AD-1290263, AD-1290264, AD-1290265, AD-1290266, AD-1290267, AD-1290268, AD-1290269, AD-1290270, AD-1334980, AD-1334981, AD-1334982, AD-1334983, AD-1334984, AD-1334985, AD-1334986, AD-1334987, AD-1334988, AD-1334989, AD-1334990, AD-1334991, AD-1334992, AD-1334993, AD-1334994, AD-1334995, AD-1334996, AD-1334997, AD-1334998, AD-1334999, AD-1335000, AD-1335001, AD-1335002, AD-1335003, AD-1335004, AD-1335005, AD-1335006, AD-1335007, AD-1335008, AD-1335009, AD-1335010, AD-1335011, AD-1335012, AD-1335013, AD-1335014, AD-1335015, AD-1335016, AD-1335017, AD-1335018, AD-1335019, AD-1335020, AD-1335021, AD-1335022, AD-1335023, AD-1335024, AD-1335025, AD-1335026, AD-1335027, AD-1335028, AD-1335029, AD-1335030, AD-1335031, AD-1335032, AD-1335033, AD-1335034, AD-1335035, AD-1335036, AD-1335037, AD-1335038, AD-1335039, AD-1335040, AD-1335041, AD-1335042, AD-1335043, AD-1335044, AD-1335045, AD-1335046, AD-1335047, AD-1335048, AD-1335049, AD-1335050, AD-1335051, AD-1335052, AD-1335053, AD-1335054, AD-1335055, AD-1335056, AD-1335057, AD-1335058, AD-1335059, AD-1335060, AD-1335061, AD-1335062, AD-1335063, AD-1335064, AD-1335065, AD-1335066, AD-1335067, AD-1335068, AD-1335069, AD-1335070, AD-1335071, AD-1335072, AD-1335073, AD-1335074, AD-1335075, AD-1335076, AD-1335077, AD-1335078, AD-1335079, AD-1335080, AD-1335081, AD-1335082, AD-1335083, AD-1335084, AD-1335085, AD-1335086, AD-1335087, AD-1335088, AD-1335089, AD-1335090, AD-1335091, AD-1335092, AD-1335093, AD-1335094, AD-1335095, AD-1335096, AD-1335097, AD-1335098, AD-1335099, AD-1335100, AD-1335101, AD-1335102, AD-1335103, AD-1335104, AD-1335105, AD-1335106, AD-1335107, AD-1335108, AD-1335109, AD-1335110, AD-1335111, AD-1335112, AD-1335113, AD-1335114, AD-1335115, AD-1335116, AD-1335117, AD-1335118, AD-1335119, AD-1335120, AD-1335121, AD-1335122, AD-1335123, AD-1335124, AD-1335125, AD-1335126, AD-1335127, AD-1335128, AD-1335129, AD-1335130, AD-1335131, AD-1335132, AD-1335133, AD-1335134, AD-1335135, AD-1335136, AD-1335137, AD-1335138, AD-1335139, AD-1335140, AD-1335141, AD-1335142, AD-1335143, AD-1335144, AD-1335145, AD-1335146, AD-1335147, AD-1335148, AD-1335149, AD-1335150, AD-1335151, AD-1335152, AD-1335153, AD-1335154, AD-1335155, AD-1335156, AD-1335157, AD-1335158, AD-1335159, AD-1335160, AD-1335161, AD-1335162, AD-1335163, AD-1335164, AD-1335165, AD-1335166, AD-1335167, AD-1335168, AD-1335169, AD-1335170, AD-1335171, AD-1335172, AD-1335173, AD-1335174, AD-1335175, AD-1335176, AD-1335177, AD-1335178, AD-1335179, AD-1335180, AD-1335181, AD-1335182, AD-1335183, AD-1335184, AD-1335185, AD-1335186, AD-1335187, AD-1335188, AD-1335189, AD-1335190, AD-1335191, AD-1335192, AD-1335193, AD-1335194, AD-1335195, AD-1335196, AD-1335197, AD-1335198, AD-1335199, AD-1335200, AD-1335201, AD-1335202, AD-1335203, AD-1335204, AD-1335205, AD-1335206, AD-1335207, AD-1335208, AD-1335209, AD-1335210, AD-1335211, AD-1335212, AD-1335213, AD-1335214, AD-1335215, AD-1335216, AD-1335217, AD-1335218, AD-1335219, AD-1335220, AD-1335221, AD-1335222, AD-1335223, AD-1335224, AD-1335225, AD-1335226, AD-1335227, AD-1335228, AD-1335229, AD-1335230, AD-1335231, AD-1335232, AD-1335233, AD-1335234, AD-1335235, AD-1335236, AD-1335237, AD-1335238, AD-1335239, AD-1335240, AD-1335241, AD-1335242, AD-1335243, AD-1335244, AD-1335245, AD-1335246, AD-1335247, AD-1335248, AD-1335249, AD-1335250, AD-1335251, AD-1335252, AD-1335253, AD-1335254, AD-1335255, AD-1335256, AD-1335257, AD-1335258, AD-1335259, AD-1335260, AD-1335261, AD-1335262, AD-1335263, AD-1335264, AD-1335265, AD-1335266, AD-1335267, AD-1335268, AD-1335269, AD-1335270, AD-1335271, AD-1335272, AD-1335273, AD-1335274, AD-1335275, AD-1335276, AD-1335277, AD-1335278, and AD-1335279.

In one embodiment, at least partial suppression of the expression of an ALK gene, is assessed by a reduction of the amount of ALK mRNA, e.g., sense mRNA, antisense mRNA, total ALK mRNA, which can be isolated from or detected in a first cell or group of cells in which an ALK gene is transcribed and which has or have been treated such that the expression of an ALK gene is inhibited, as compared to a second cell or group of cells substantially identical to the first cell or group of cells but which has or have not been so treated (control cells). The degree of inhibition may be expressed in terms of:

$$\frac{\left(\text{mRNA in control cells}\right) \text{-} \left(\text{mRNA in treated cells}\right)}{\left(\text{mRNA in control cells}\right)} \cdot 100 \%$$

The phrase “contacting a cell with an RNAi agent,” such as a dsRNA, as used herein, includes contacting a cell by any possible means. Contacting a cell with an RNAi agent includes contacting a cell in vitro with the RNAi agent or contacting a cell in vivo with the RNAi agent. The contacting may be done directly or indirectly. Thus, for example, the RNAi agent may be put into physical contact with the cell by the individual performing the method, or alternatively, the RNAi agent may be put into a situation that will permit or cause it to subsequently come into contact with the cell.

Contacting a cell in vitro may be done, for example, by incubating the cell with the RNAi agent. Contacting a cell in vivo may be done, for example, by injecting the RNAi agent into or near the tissue where the cell is located, or by injecting the RNAi agent into another area, e.g., the central nervous system (CNS), optionally via intrathecal, intravitreal or other injection, or to the bloodstream or the subcutaneous space, such that the agent will subsequently reach the tissue where the cell to be contacted is located. For example, the RNAi agent may contain or be coupled to a ligand, e.g., a lipophilic moiety or moieties as described below and further detailed, e.g., in PCT/US2019/031170, which is incorporated herein by reference, that directs or otherwise stabilizes the RNAi agent at a site of interest, e.g., the CNS. Combinations of in vitro and in vivo methods of contacting are also possible. For example, a cell may also be contacted in vitro with an RNAi agent and subsequently transplanted into a subject.

In one embodiment, contacting a cell with an RNAi agent includes “introducing” or “delivering the RNAi agent into the cell” by facilitating or effecting uptake or absorption into the cell. Absorption or uptake of an RNAi agent can occur through unaided diffusive or active cellular processes, or by auxiliary agents or devices. Introducing an RNAi agent into a cell may be in vitro or in vivo. For example, for in vivo introduction, an RNAi agent can be injected into a tissue site or administered systemically. In vitro introduction into a cell includes methods known in the art such as electroporation and lipofection. Further approaches are described herein below or are known in the art.

The term “lipophile” or “lipophilic moiety” broadly refers to any compound or chemical moiety having an affinity for lipids. One way to characterize the lipophilicity of the lipophilic moiety is by the octanol-water partition coefficient, logK_{ow}, where K_{ow} is the ratio of a chemical’s concentration in the octanol-phase to its concentration in the aqueous phase of a two-phase system at equilibrium. The octanol-water partition coefficient is a laboratory-measured property of a substance. However, it may also be predicted by using coefficients attributed to the structural components of a chemical which are calculated using first-principle or empirical methods (see, for example, Tetko et al., J. Chem. Inf. Comput. Sci. 41:1407-21 (2001), which is incorporated herein by reference in its entirety). It provides a thermodynamic measure of the tendency of the substance to prefer a non-aqueous or oily milieu rather than water (i.e. its hydrophilic/lipophilic balance). In principle, a chemical substance is lipophilic in character when its logK_{ow} exceeds 0. Typically, the lipophilic moiety possesses a logK_{ow} exceeding 1, exceeding 1.5, exceeding 2, exceeding 3, exceeding 4, exceeding 5, or exceeding 10. For instance, the logK_{ow} of 6-amino hexanol, for instance, is predicted to be approximately 0.7. Using the same method, the logK_{ow} of cholesteryl N-(hexan-6-ol) carbamate is predicted to be 10.7.

The lipophilicity of a molecule can change with respect to the functional group it carries. For instance, adding a hydroxyl group or amine group to the end of a lipophilic moiety can increase or decrease the partition coefficient (e.g., logK_{ow}) value of the lipophilic moiety.

Alternatively, the hydrophobicity of the double-stranded RNAi agent, conjugated to one or more lipophilic moieties, can be measured by its protein binding characteristics. For instance, in certain embodiments, the unbound fraction in the plasma protein binding assay of the double-stranded RNAi agent could be determined to positively correlate to the relative hydrophobicity of the double-stranded RNAi agent, which could then positively correlate to the silencing activity of the double-stranded RNAi agent.

In one embodiment, the plasma protein binding assay determined is an electrophoretic mobility shift assay (EMSA) using human serum albumin protein. An exemplary protocol of this binding assay is illustrated in detail in, e.g., PCT/US2019/031170. The hydrophobicity of the double-stranded RNAi agent, measured by fraction of unbound siRNA in the binding assay, exceeds 0.15, exceeds 0.2, exceeds 0.25, exceeds 0.3, exceeds 0.35, exceeds 0.4, exceeds 0.45, or exceeds 0.5 for an enhanced in vivo delivery of siRNA.

Accordingly, conjugating the lipophilic moieties to the internal position(s) of the double-stranded RNAi agent provides optimal hydrophobicity for the enhanced in vivo delivery of siRNA.

The term “lipid nanoparticle” or “LNP” is a vesicle comprising a lipid layer encapsulating a pharmaceutically active molecule, such as a nucleic acid molecule, e.g., a rNAi agent or a plasmid from which an RNAi agent is transcribed. LNPs are described in, for example, U.S. Pat. Nos. 6,858,225, 6,815,432, 8,158,601, and 8,058,069, the entire contents of which are hereby incorporated herein by reference.

As used herein, a “subject” is an animal, such as a mammal, including a primate (such as a human, a non-human primate, e.g., a monkey, and a chimpanzee), or a non-primate (such as a rat, or a mouse). In one embodiment, the subject is a human, such as a human being treated or assessed for a disease, disorder, or condition that would benefit from reduction in ALK expression; a human at risk for a disease, disorder, or condition that would benefit from reduction in ALK expression; a human having a disease, disorder, or condition that would benefit from reduction in ALK expression; or human being treated for a disease, disorder, or condition that would benefit from reduction in ALK expression as described herein. In some embodiments, the subject is a female human. In other embodiments, the subject is a male human. In one embodiment, the subject is an adult subject. In one embodiment, the subject is a pediatric subject. In another embodiment, the subject is a juvenile subject, i.e., a subject below 20 years of age.

As used herein, the terms “treating” or “treatment” refer to a beneficial or desired result including, but not limited to, alleviation or amelioration of one or more signs or symptoms associated with ALK gene expression or ALK protein production, e.g., ALK-associated diseases, such as type 2 diabetes, obesity, and obesity-associated disease. “Treatment” can also mean prolonging survival as compared to expected survival in the absence of treatment.

The term “lower” in the context of the level of ALK in a subject or a disease marker or symptom refers to a statistically significant decrease in such level. The decrease can be, for example, at least 10%, 15%, 20%, 25%, 30%, %, 40%, 45%, 50%, 55%, 60%, 65%, 70%, 75%, 80%, 85%, 90%, 95%, or more. In certain embodiments, a decrease is at least about 20%. In certain embodiments, the decrease is at least about 30% in a disease marker, e.g., a decrease of 30%, 35%, 40%, 45%, 50%, 55%, 60%, 65%, 70%, 75%, 80%, 85%, 90%, 95%, 99%, or more. In certain embodiments, the decrease is at least about 50% in a disease marker. “Lower” in the context of the level of ALK in a subject is preferably down to a level accepted as within the range of normal for an individual without such disorder. In certain embodiments, “lower” is the decrease in the difference between the level of a marker or symptom for a subject suffering from a disease and a level accepted within the range of normal for an individual, e.g., the level of decrease in bodyweight between an obese individual and an individual having a weight accepted within the range of normal.

As used herein, “prevention” or “preventing,” when used in reference to a disease, disorder, or condition thereof, that would benefit from a reduction in expression of an ALK gene or production of an ALK protein, refers to a reduction in the likelihood that a subject will develop a symptom or a sign associated with such a disease, disorder, or condition, e.g., a symptom or a sign of an ALK-associated disease, such as type 2 diabetes, obesity, or obesity-associated disease. The failure to develop a disease, disorder, or condition, or the reduction in the development of a symptom associated with such a disease, disorder, or condition (e.g., by at least about 10% on a clinically accepted scale for that disease or disorder), or the exhibition of delayed symptoms delayed (e.g., by days, weeks, months or years) is considered effective prevention.

As used herein, the term “ALK-associated disease,” “ALK-associated condition,” or “ALK-associated disorder” includes any disease, condition, or disorder that would benefit from reduction in the expression and/or activity of ALK. Exemplary ALK-associated conditions include type 2 diabetes, obesity, obesity-associated disorders, being overweight, being in need or desire of losing weight, and being in need or desire of supporting weight maintenance or preventing further weight gain.

As used herein, the term “obese” is defined for a human as having a BMI greater than 30. The term “body mass index” or “BMI” as used herein means the ratio of weight in kg divided by the height in meters, squared. The term “normal weight” as used herein is defined for a human as having a BMI of 18.5 to 25, whereas the term “underweight” as used herein may be defined as a BMI of less than 18.5. As used herein, the term “overweight” is defined for an adult human as having a BMI between 25 and 30.

As used herein, the term “obesity” refers to a condition in which the natural energy reserve, stored in the fatty tissue of animals, in particular humans and other mammals, is increased to a point where it is associated with certain health conditions or increased mortality.

Obesity may be caused by lifestyle factors, e.g., high calorie intake and insufficient exercise. There are also genetic disorders that can cause obesity, e.g., mutations of the MC4R, LEP, or POMC, genes, Bardet-Biedl syndrome, and Prader-Willi syndrome.

As used herein, the term “obesity-associated disorder,” used interchangeably with “obesity-associated condition” or “obesity-associated disease,” includes any condition which an obese individual is at an increased risk of developing, e.g., type 2 diabetes, hypertension, dyslipidemia, atherosclerosis, coronary heart disease, stroke, metabolic syndrome, gallbladder disease, fatty liver, osteoarthritis, sleep apnea, breathing problems, various types of cancer (e.g., endometrial cancer, esophageal adenocarcinoma, gastric cardia cancer, liver cancer, kidney cancer, pancreatic cancer), mental illness (e.g., depression, anxiety), body pain, and difficulty with physical functioning.

As used herein, the term “weight loss” or “losing weight” may refer to a reduction in parameters such as weight (e.g., in kilograms), BMI (kg/m²), waist-hip ratio, fat mass (e.g., in kilograms), hip circumference (e.g. in centimeters) or waist circumference (e.g. in centimeters). Weight loss may be calculated by subtracting the value of one or more of the aforementioned parameters at the end of an intervention (e.g., a diet and/or exercise regimen) from the value of the parameter at the onset of the weight loss intervention. The degree of weight loss may be expressed as a percent change of one of the aforementioned weight phenotype parameters (e.g., a percent change in a subject’s body weight (e.g., in kilograms) or BMI (kg/m²)). For example, a subject may lose at least 10% of their initial body weight, at least 8% of their initial body weight, or at least 5% of their initial body weight. By way of example only, a subject may lose between 5 and 10% of their initial body weight. In one embodiment, a degree of weight loss of at least 10% of initial body weight results in a considerable decrease in the risk of obesity-related diseases.

As used herein, the term “weight maintenance” may refer to the maintenance in parameters such as weight (e.g., in kilograms), BMI (kg/m²), waist-hip ratio, fat mass (e.g., in kilograms), hip circumference (e.g., in centimeters), or waist circumference (e.g., in centimeters). Weight maintenance may refer to, for example, maintaining weight lost following an intervention (e.g., a diet and/or exercise regimen). The degree of weight maintenance may be calculated by determining the change in one or more of the afore-mentioned parameters over a period of time. The period of time may be, for example, at least 5, 10, 15, 20, 25, 30, 35, 40, 45 or 50 weeks. Weight maintenance supported by the agents of the invention may result in, for example, a change (e.g., gain) of less than 10%, 9%, 8%, 7%, 6%, 5%, 4%, 3%, 2% or 1% in one or more of the afore-mentioned parameters over a period of time. The degree of weight maintenance may be expressed as the weight regained during a period following attainment of weight loss, for example as a percentage of the weight lost during attainment of weight loss. In particular, the agents of the invention may support weight maintenance during and/or following a period of weight loss intervention (e.g., a diet or exercise regime). In one aspect, the invention provides the non-therapeutic use of an agent of the invention to maintain a healthy body composition, for example after a period of weight loss.

“Therapeutically effective amount,” as used herein, is intended to include the amount of an RNAi agent that, when administered to a subject having an ALK-associated disease, is sufficient to effect treatment of the disease (e.g., by diminishing, ameliorating, or maintaining the existing disease or one or more symptoms of disease). The “therapeutically effective amount” may vary depending on the RNAi agent, how the agent is administered, the disease and its severity and the history, age, weight, family history, genetic makeup, the types of preceding or concomitant treatments, if any, and other individual characteristics of the subject to be treated.

“Prophylactically effective amount,” as used herein, is intended to include the amount of an RNAi agent that, when administered to a subject having an ALK-associated disorder, is sufficient to prevent or ameliorate the disease or one or more symptoms of the disease. Ameliorating the disease includes slowing the course of the disease or reducing the severity of later-developing disease. The “prophylactically effective amount” may vary depending on the RNAi agent, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of an RNAi agent that produces some desired local or systemic effect at a reasonable benefit/risk ratio applicable to any treatment. An RNAi agent employed in the methods of the present disclosure may be administered in a sufficient amount to produce a reasonable benefit/risk ratio applicable to such treatment.

The phrase “pharmaceutically acceptable” is employed herein to refer to those compounds, materials, compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer’s solution; (19) ethyl alcohol; (20) pH buffered solutions; (21) polyesters, polycarbonates or polyanhydrides; (22) bulking agents, such as polypeptides and amino acids (23) serum component, such as serum albumin, HDL and LDL; and (22) other non-toxic compatible substances employed in pharmaceutical formulations.

The term “sample,” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs or localized regions. For example, samples may be derived from particular organs, parts of organs, or fluids or cells within those organs. In certain embodiments, samples may be derived from the brain (e.g., whole brain or certain segments of brain, e.g., striatum, or certain types of cells in the brain, such as, e.g., neurons and glial cells (astrocytes, oligodendrocytes, microglial cells)). In some embodiments, a “sample derived from a subject” refers to blood drawn from the subject or plasma or serum derived therefrom. In further embodiments, a “sample derived from a subject” refers to brain tissue (or subcomponents thereof) or retinal tissue (or subcomponents thereof) derived from the subject.

### II. RNAi Agents of the Disclosure

Described herein are RNAi agents which inhibit the expression of an ALK gene. In one embodiment, the RNAi agent includes double stranded ribonucleic acid (dsRNA) molecules for inhibiting the expression of an ALK gene in a cell, such as a cell within a subject, e.g., a mammal, such as a human having an ALK-associated disease, e.g., type 2 diabetes, obesity, or an obesity-associated disease. The dsRNA includes an antisense strand having a region of complementarity which is complementary to at least a part of an mRNA formed in the expression of an ALK gene. The region of complementarity is about 15-30 nucleotides or less in length. Upon contact with a cell expressing the ALK gene, the RNAi agent inhibits the expression of the ALK gene (e.g., a human gene, a primate gene, a non-primate gene) by at least 30% as assayed by, for example, a PCR or branched DNA (bDNA)-based method, or by a protein-based method, such as by immunofluorescence analysis, using, for example, western blotting or flowcytometric techniques. In one embodiment, the level of knockdown is assayed in monkey Cos-7 cells using an assay method provided in Example 2 below. In another embodiment, the level of knockdown is assayed in human BE(2)-C cells. In some embodiments, the level of knockdown is assayed in mouse Neuro-2a cells.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of an ALK gene. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. In certain embodiments, the duplex structure is 18 to 25 base pairs in length, e.g., 18-25, 18-24, 18-23, 18-22, 18-21, 18-20, 19-25, 19-24, 19-23, 19-22, 19-21, 19-20, 20-25, 20-24,20-23, 20-22, 20-21, 21-25, 21-24, 21-23, 21-22, 22-25, 22-24, 22-23, 23-25, 23-24 or 24-25 base pairs in length, for example, 19-21 basepairs in length. Ranges and lengths intermediate to the above recited ranges and lengths are also contemplated to be part of the disclosure.

Similarly, the region of complementarity to the target sequence is 15 to 30 nucleotides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleotides in length, for example 19-23 nucleotides in length or 21-23 nucleotides in length. Ranges and lengths intermediate to the above recited ranges and lengths are also contemplated to be part of the disclosure.

In some embodiments, the duplex structure is 19 to 30 base pairs in length. Similarly, the region of complementarity to the target sequence is 19 to 30 nucleotides in length.

In some embodiments, the dsRNA is 15 to 23 nucleotides in length, 19 to 23 nucleotides in length, or 25 to 30 nucleotides in length. In general, the dsRNA is long enough to serve as a substrate for the Dicer enzyme. For example, it is well known in the art that dsRNAs longer than about 21-23 nucleotides can serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 15 to 36 base pairs, e.g., 15-36, 15-35, 15-34, 15-33, 15-32, 15-31, 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs, for example, 19-21 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, an RNAi agent useful to target ALK expression is not generated in the target cell by cleavage of a larger dsRNA.

A dsRNA as described herein can further include one or more single-stranded nucleotide overhangs e.g., 1, 2, 3, or 4 nucleotides. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′-end, 3′-end or both ends of either an antisense or sense strand of a dsRNA.

A dsRNA can be synthesized by standard methods known in the art. Double stranded RNAi compounds of the invention may be prepared using a two-step procedure. First, the individual strands of the double stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Similarly, single-stranded oligonucleotides of the invention can be prepared using solution-phase or solid-phase organic synthesis or both.

In one aspect, a dsRNA of the disclosure includes at least two nucleotide sequences, a sense sequence and an antisense sequence. The sense strand sequence for ALK may be selected from the group of sequences provided in Tables 2, 3, or 4, and the corresponding nucleotide sequence of the antisense strand of the sense strand may be selected from the group of sequences of Tables 2, 3, or 4. In this aspect, one of the two sequences is complementary to the other of the two sequences, with one of the sequences being substantially complementary to a sequence of an mRNA generated in the expression of an ALK gene. As such, in this aspect, a dsRNA will include two oligonucleotides, where one oligonucleotide is described as the sense strand (passenger strand) in Tables 2, 3, or 4, and the second oligonucleotide is described as the corresponding antisense strand (guide strand) of the sense strand in Tables 2, 3, or 4.

In one embodiment, the substantially complementary sequences of the dsRNA are contained on separate oligonucleotides. In another embodiment, the substantially complementary sequences of the dsRNA are contained on a single oligonucleotide.

It will be understood that, although the sequences in Tables 2, 3, and 4 are described as modified or conjugated sequences, the RNA of the RNAi agent of the disclosure e.g., a dsRNA of the disclosure, may comprise any one of the sequences set forth in Tables 2, 3, or 4 that is un-modified, unconjugated, or modified or conjugated differently than described therein. For example, although the sense strands of the agents of the invention may be conjugated to a GalNAc ligand, these agents may be conjugated to a moiety that directs delivery to the CNS, e.g., a C16 ligand, as described herein. A lipophilic ligand can be included in any of the positions provided in the instant application.

The skilled person is well aware that dsRNAs having a duplex structure of about 20 to 23 base pairs, e.g., 21, base pairs have been hailed as particularly effective in inducing RNA interference (Elbashir et al., (2001) EMBO J., 20:6877-6888). However, others have found that shorter or longer RNA duplex structures can also be effective (Chu and Rana (2007) RNA 14: 1714-1719; Kim et al. (2005) Nat Biotech 23:222-226). In the embodiments described above, by virtue of the nature of the oligonucleotide sequences provided herein, dsRNAs described herein can include at least one strand of a length of minimally 21 nucleotides. It can be reasonably expected that shorter duplexes minus only a few nucleotides on one or both ends can be similarly effective as compared to the dsRNAs described above. Hence, dsRNAs having a sequence of at least 15, 16, 17, 18, 19, 20, or more contiguous nucleotides derived from one of the sequences provided herein, and differing in their ability to inhibit the expression of an ALK gene by not more than 10, 15, 20, 25, 30, 35, 40, 45 or 50% inhibition from a dsRNA comprising the full sequence using the in vitro assay with, e.g., A549 cells and a 10 nM concentration of the RNA agent and the PCR assay as provided in the examples herein, are contemplated to be within the scope of the present disclosure. In some embodiments, inhibition from a dsRNA comprising the full sequence was measured using the in vitro assay with primary mouse hepatocytes.

In addition, the RNAs described herein identify a site(s) in an ALK transcript that is susceptible to RISC-mediated cleavage. As such, the present disclosure further features RNAi agents that target within this site(s). As used herein, an RNAi agent is said to target within a particular site of an RNA transcript if the RNAi agent promotes cleavage of the transcript anywhere within that particular site. Such an RNAi agent will generally include at least about 15 contiguous nucleotides, preferably at least 19 nucleotides, from one of the sequences provided herein coupled to additional nucleotide sequences taken from the region contiguous to the selected sequence in an ALK gene.

### III. Modified RNAi Agents of the Disclosure

In one embodiment, the RNA of the RNAi agent of the disclosure e.g., a dsRNA, is un-modified, and does not comprise, e.g., chemical modifications or conjugations known in the art and described herein. In one embodiment, the RNA of an RNAi agent of the disclosure, e.g., a dsRNA, is chemically modified to enhance stability or other beneficial characteristics. In certain embodiments of the disclosure, substantially all of the nucleotides of an RNAi agent of the disclosure are modified. In other embodiments of the disclosure, all of the nucleotides of an RNAi agent of the disclosure are modified. RNAi agents of the disclosure in which “substantially all of the nucleotides are modified” are largely but not wholly modified and can include not more than 5, 4, 3, 2, or unmodified nucleotides. In still other embodiments of the disclosure, RNAi agents of the disclosure can include not more than 5, 4, 3, 2 or 1 modified nucleotides.

The nucleic acids featured in the disclosure can be synthesized or modified by methods well established in the art, such as those described in “Current protocols in nucleic acid chemistry,” Beaucage, S.L. et al. (Edrs.), John Wiley & Sons, Inc., New York, NY, USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. Specific examples of RNAi agents useful in the embodiments described herein include, but are not limited to, RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments, a modified RNAi agent will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments of the invention, the dsRNA agents of the invention are in a free acid form. In other embodiments of the invention, the dsRNA agents of the invention are in a salt form. In one embodiment, the dsRNA agents of the invention are in a sodium salt form. In certain embodiments, when the dsRNA agents of the invention are in the sodium salt form, sodium ions are present in the agent as counterions for substantially all of the phosphodiester and/or phosphorothioate groups present in the agent. Agents in which substantially all of the phosphodiester and/or phosphorothioate linkages have a sodium counterion include not more than 5, 4, 3, 2, or 1 phosphodiester and/or phosphorothioate linkages without a sodium counterion. In some embodiments, when the dsRNA agents of the invention are in the sodium salt form, sodium ions are present in the agent as counterions for all of the phosphodiester and/or phosphorothioate groups present in the agent.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, the entire contents of each of which are hereby incorporated herein by reference.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

In other embodiments, suitable RNA mimetics are contemplated for use in RNAi agents, in which both the sugar and the internucleoside linkage, i.e., the backbone, of the nucleotide units are replaced with novel groups. The base units are maintained for hybridization with an appropriate nucleic acid target compound. One such oligomeric compound, a RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the RNAi agents of the disclosure are described in, for example, in Nielsen et al., Science, 1991, 254, 1497-1500.

Some embodiments featured in the disclosure include RNAs with phosphorothioate backbones and oligonucleosides with heteroatom backbones, and in particular —CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂— and —N(CH₃)—CH₂—CH₂—[wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced US5,034,506.

Modified RNAs can also contain one or more substituted sugar moieties. The RNAi agents, e.g., dsRNAs, featured herein can include one of the following at the 2′-position: OH; F; O—, S—, or N—alkyl; O—, S—, or N-alkenyl; O—, S— or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO] ₘCH₃, O(CH₂).ₙOCH₃, O(CH₂)ₙNH₂, O(CH₂) ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an RNAi agent, or a group for improving the pharmacodynamic properties of an RNAi agent, and other substituents having similar properties. In some embodiments, the modification includes a 2′-methoxyethoxy (2′-O--CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O--CH₂--O--CH₂--N(CH₂)₂. Further exemplary modifications include: 5′-Me-2′-F nucleotides, 5′-Me-2′-OMe nucleotides, 5′-Me-2′-deoxynucleotides, (both Rand S isomers in these three families); 2′-alkoxyalkyl; and 2′-NMA (N-methylacetamide).

Other modifications include 2′-methoxy (2′-OCH₃), 2′-aminopropoxy (2′-OCH₂CH₂CH₂NH₂), 2′-O-hexadecyl, and 2′-fluoro (2′-F). Similar modifications can also be made at other positions on the RNA of an RNAi agent, particularly the 3′ position of the sugar on the 3′ terminal nucleotide or in 2′-5′ linked dsRNAs and the 5′ position of 5′ terminal nucleotide. RNAi agents can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

An RNAi agent of the disclosure can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30:613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the disclosure. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

An RNAi agent of the disclosure can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mol Canc Ther 6(3):833-843; Grunweller, A. et al., (2003) Nucleic Acids Research 31(12):3185-3193).

An RNAi agent of the disclosure can also be modified to include one or more bicyclic sugar moieties. A “bicyclic sugar” is a furanosyl ring modified by the bridging of two atoms. A “bicyclic nucleoside” (“BNA”) is a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring. Thus, in some embodiments an agent of the disclosure may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH2-O-2′ bridge. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mol Canc Ther 6(3):833-843; Grunweller, A. et al., (2003) Nucleic Acids Research 31(12):3185-3193). Examples of bicyclic nucleosides for use in the polynucleotides of the disclosure include without limitation nucleosides comprising a bridge between the 4′ and the 2′ ribosyl ring atoms. In certain embodiments, the antisense polynucleotide agents of the disclosure include one or more bicyclic nucleosides comprising a 4′ to 2′ bridge. Examples of such 4′ to 2′ bridged bicyclic nucleosides, include but are not limited to 4′-(CH2)—O-2′ (LNA); 4′-(CH2)—S-2′; 4′-(CH2)2—O-2′ (ENA); 4′-CH(CH3)—O-2′ (also referred to as “constrained ethyl” or “cEt”) and 4′-CH(CH2OCH3)—O-2′ (and analogs thereof; see, e.g., U.S. Pat. No. 7,399,845); 4′-C(CH3)(CH3)—O-2′ (and analogs thereof; see e.g., U.S. Pat. No. 8,278,283); 4′-CH2-N(OCH3)-2′ (and analogs thereof; see e.g., U.S. Pat. No. 8,278,425); 4′-CH2—O—N(CH3)-2′ (see, e.g., U.S. Pat. Publication No. 2004/0171570); 4′-CH2—N(R)—O-2′, wherein R is H, C1-C12 alkyl, or a protecting group (see, e.g., U.S. Pat. No. 7,427,672); 4′-CH2—C(H)(CH3)-2′ (see, e.g., Chattopadhyaya et al., J. Org. Chem., 2009, 74, 118-134); and 4′-CH2—C(=CH2)-2′ (and analogs thereof; see, e.g., U.S. Pat. No. 8,278,426). The entire contents of each of the foregoing are hereby incorporated herein by reference.

Additional representative US Patents and US Patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133;7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemicalsugar configurations including for example α-L-ribofuranose and β-D-ribofuranose (see WO 99/14226).

An RNAi agent of the disclosure can also be modified to include one or more constrained ethyl nucleotides. As used herein, a “constrained ethyl nucleotide” or “cEt” is a locked nucleic acid comprising a bicyclic sugar moiety comprising a 4′=-CH(CH3)-0-2′ bridge. In one embodiment, a constrained ethyl nucleotide is in the S conformation referred to herein as “S-cEt.”

An RNAi agent of the disclosure may also include one or more “conformationally restricted nucleotides” (“CRN”). CRN are nucleotide analogs with a linker connecting the C2′and C4′ carbons of ribose or the C3 and -C5’ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US 2013/0190383; and WO 2013/036868, the entire contents of each of which are hereby incorporated herein by reference.

In some embodiments, an RNAi agent of the disclosure comprises one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1’-C4’ have been removed (i.e. the covalent carbon-oxygen-carbon bond between the C1′ and C4′ carbons). In another example, the C2’-C3’ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, US8,314,227; and U.S. Pat. Publication Nos. 2013/0096289; 2013/0011922; and 2011/0313020, the entire contents of each of which are hereby incorporated herein by reference.

Potentially stabilizing modifications to the ends of RNA molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-0-deoxythymidine (ether), N-(aminocaproyl)-4-hydroxyprolinol (Hyp-C6-amino), 2-docosanoyl-uridine-3″- phosphate, inverted base dT(idT) and others. Disclosure of this modification can be found in WO 2011/005861.

Other modifications of an RNAi agent of the disclosure include a 5′ phosphate or 5′ phosphate mimic, e.g., a 5′-terminal phosphate or phosphate mimic on the antisense strand of an RNAi agent. Suitable phosphate mimics are disclosed in, for example US 2012/0157511, the entire contents of which are incorporated herein by reference.

### A. Modified RNAi Agents Comprising Motifs of the Disclosure

In certain aspects of the disclosure, the double-stranded RNAi agents of the disclosure include agents with chemical modifications as disclosed, for example, in WO 2013/075035, the entire contents of which are incorporated herein by reference. As shown herein and in WO 2013/075035, a superior result may be obtained by introducing one or more motifs of three identical modifications on three consecutive nucleotides into a sense strand or antisense strand of an RNAi agent, particularly at or near the cleavage site. In some embodiments, the sense strand and antisense strand of the RNAi agent may otherwise be completely modified. The introduction of these motifs interrupts the modification pattern, if present, of the sense or antisense strand. The RNAi agent may be optionally conjugated with a lipophilic ligand, e.g., a C16 ligand, for instance on the sense strand. The RNAi agent may be optionally modified with a (S)-glycol nucleic acid (GNA) modification, for instance on one or more residues of the antisense strand. The resulting RNAi agents present superior gene silencing activity.

Accordingly, the disclosure provides double stranded RNAi agents capable of inhibiting the expression of a target gene (i.e., an ALK gene) in vivo. The RNAi agent comprises a sense strand and an antisense strand. Each strand of the RNAi agent may be 15-30 nucleotides in length. For example, each strand may be 16-30 nucleotides in length, 17-30 nucleotides in length, 25-30 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length. In certain embodiments, each strand is 19-23 nucleotides in length.

The sense strand and antisense strand typically form a duplex double stranded RNA (“dsRNA”), also referred to herein as an “RNAi agent.” The duplex region of an RNAi agent may be 15-30 nucleotide pairs in length. For example, the duplex region can be 16-30 nucleotide pairs in length, 17-30 nucleotide pairs in length, 27-30 nucleotide pairs in length, 17 - 23 nucleotide pairs in length, 17-21 nucleotide pairs in length, 17-19 nucleotide pairs in length, 19-25 nucleotide pairs in length, 19-23 nucleotide pairs in length, 19- 21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotides in length. In another embodiment, the duplex region is 19-21 nucleotide pairs in length.

In one embodiment, the RNAi agent may contain one or more overhang regions or capping groups at the 3′-end, 5′-end, or both ends of one or both strands. The overhang can be 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. In another embodiment, the nucleotide overhang region is 2 nucleotides in length. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In one embodiment, the nucleotides in the overhang region of the RNAi agent can each independently be a modified or unmodified nucleotide including, but no limited to 2′-sugar modified, such as, 2-F, 2′-O-methyl, thymidine (T), and any combinations thereof.

For example, TT can be an overhang sequence for either end on either strand. The overhang can form a mismatch with the target mRNA or it can be complementary to the gene sequences being targeted or can be another sequence.

The 5′ - or 3′- overhangs at the sense strand, antisense strand or both strands of the RNAi agent may be phosphorylated. In some embodiments, the overhang region(s) contains two nucleotides having a phosphorothioate between the two nucleotides, where the two nucleotides can be the same or different. In one embodiment, the overhang is present at the 3′-end of the sense strand, antisense strand, or both strands. In one embodiment, this 3′-overhang is present in the antisense strand. In one embodiment, this 3′-overhang is present in the sense strand.

The RNAi agent may contain only a single overhang, which can strengthen the interference activity of the RNAi, without affecting its overall stability. For example, the single-stranded overhang may be located at the 3′-terminal end of the sense strand or, alternatively, at the 3′-terminal end of the antisense strand. The RNAi may also have a blunt end, located at the 5′-end of the antisense strand (or the 3′-end of the sense strand) or vice versa. Generally, the antisense strand of the RNAi has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In one embodiment, the RNAi agent is a double ended bluntmer of 19 nucleotides in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, 13 from the 5′end.

In another embodiment, the RNAi agent is a double ended bluntmer of 20 nucleotides in length, wherein the sense strand contains at least one motif of three 2′-F modifications on three consecutive nucleotides at positions 8, 9, 10 from the 5′end. The antisense strand contains at least one motif of three 2′-O-methyl modifications on three consecutive nucleotides at positions 11, 12, 13 from the 5′end.

In yet another embodiment, the RNAi agent is a double ended bluntmer of 21 nucleotides in length, wherein the sense strand contains at least one motif of three 2′-F modifications on three consecutive nucleotides at positions 9, 10, 11 from the 5′end. The antisense strand contains at least one motif of three 2′-O-methyl modifications on three consecutive nucleotides at positions 11, 12, 13 from the 5′end.

In one embodiment, the RNAi agent comprises a 21 nucleotide sense strand and a 23 nucleotide antisense strand, wherein the sense strand contains at least one motif of three 2′-F modifications on three consecutive nucleotides at positions 9, 10, 11 from the 5′end; the antisense strand contains at least one motif of three 2′-O-methyl modifications on three consecutive nucleotides at positions 11, 12, 13 from the 5′end, wherein one end of the RNAi agent is blunt, while the other end comprises a 2 nucleotide overhang. Preferably, the 2 nucleotide overhang is at the 3′-end of the antisense strand. When the 2 nucleotide overhang is at the 3′-end of the antisense strand, there may be two phosphorothioate internucleotide linkages between the terminal three nucleotides, wherein two of the three nucleotides are the overhang nucleotides, and the third nucleotide is a paired nucleotide next to the overhang nucleotide. In one embodiment, the RNAi agent additionally has two phosphorothioate internucleotide linkages between the terminal three nucleotides at both the 5′-end of the sense strand and at the 5′-end of the antisense strand. In one embodiment, every nucleotide in the sense strand and the antisense strand of the RNAi agent, including the nucleotides that are part of the motifs are modified nucleotides. In one embodiment each residue is independently modified with a 2′-O-methyl or 3′-fluoro, e.g., in an alternating motif. Optionally, the RNAi agent further comprises a ligand (e.g., a lipophilic ligand, optionally a C16 ligand).

In one embodiment, the RNAi agent comprises a sense and an antisense strand, wherein the sense strand is 25-30 nucleotide residues in length, wherein starting from the 5′ terminal nucleotide (position 1) positions 1 to 23 of the first strand comprise at least 8 ribonucleotides; the antisense strand is 36-66 nucleotide residues in length and, starting from the 3′ terminal nucleotide, comprises at least 8 ribonucleotides in the positions paired with positions 1- 23 of sense strand to form a duplex; wherein at least the 3 ‘ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when the double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In one embodiment, the RNAi agent comprises sense and antisense strands, wherein the RNAi agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein the 3′ end of the first strand and the 5′ end of the second strand form a blunt end and the second strand is 1-4 nucleotides longer at its 3′ end than the first strand, wherein the duplex region which is at least 25 nucleotides in length, and the second strand is sufficiently complementary to a target mRNA along at least 19 nucleotide of the second strand length to reduce target gene expression when the RNAi agent is introduced into a mammalian cell, and wherein dicer cleavage of the RNAi agent preferentially results in an siRNA comprising the 3′ end of the second strand, thereby reducing expression of the target gene in the mammal. Optionally, the RNAi agent further comprises a ligand.

In one embodiment, the sense strand of the RNAi agent contains at least one motif of three identical modifications on three consecutive nucleotides, where one of the motifs occurs at the cleavage site in the sense strand.

In one embodiment, the antisense strand of the RNAi agent can also contain at least one motif of three identical modifications on three consecutive nucleotides, where one of the motifs occurs at or near the cleavage site in the antisense strand.

For an RNAi agent having a duplex region of 17-23 nucleotide in length, the cleavage site of the antisense strand is typically around the 10, 11 and 12 positions from the 5′-end. Thus the motifs of three identical modifications may occur at the 9, 10, 11 positions; 10, 11, 12 positions; 11, 12, 13 positions; 12, 13, 14 positions; or 13, 14, 15 positions of the antisense strand, the count starting from the 1^{st} nucleotide from the 5′-end of the antisense strand, or, the count starting from the 1^{st} paired nucleotide within the duplex region from the 5′- end of the antisense strand. The cleavage site in the antisense strand may also change according to the length of the duplex region of the RNAi from the 5′-end.

The sense strand of the RNAi agent may contain at least one motif of three identical modifications on three consecutive nucleotides at the cleavage site of the strand; and the antisense strand may have at least one motif of three identical modifications on three consecutive nucleotides at or near the cleavage site of the strand. When the sense strand and the antisense strand form a dsRNA duplex, the sense strand and the antisense strand can be so aligned that one motif of the three nucleotides on the sense strand and one motif of the three nucleotides on the antisense strand have at least one nucleotide overlap, i.e., at least one of the three nucleotides of the motif in the sense strand forms a base pair with at least one of the three nucleotides of the motif in the antisense strand. Alternatively, at least two nucleotides may overlap, or all three nucleotides may overlap.

In one embodiment, the sense strand of the RNAi agent may contain more than one motif of three identical modifications on three consecutive nucleotides. The first motif may occur at or near the cleavage site of the strand and the other motifs may be a wing modification. The term “wing modification” herein refers to a motif occurring at another portion of the strand that is separated from the motif at or near the cleavage site of the same strand. The wing modification is either adjacent to the first motif or is separated by at least one or more nucleotides. When the motifs are immediately adjacent to each other, then the chemistry of the motifs are distinct from each other and when the motifs are separated by one or more nucleotide than the chemistries can be the same or different. Two or more wing modifications may be present. For instance, when two wing modifications are present, each wing modification may occur at one end relative to the first motif which is at or near cleavage site or on either side of the lead motif.

Like the sense strand, the antisense strand of the RNAi agent may contain more than one motif of three identical modifications on three consecutive nucleotides, with at least one of the motifs occurring at or near the cleavage site of the strand. This antisense strand may also contain one or more wing modifications in an alignment similar to the wing modifications that may be present on the sense strand.

In one embodiment, the wing modification on the sense strand or antisense strand of the RNAi agent typically does not include the first one or two terminal nucleotides at the 3′-end, 5′-end or both ends of the strand.

In another embodiment, the wing modification on the sense strand or antisense strand of the RNAi agent typically does not include the first one or two paired nucleotides within the duplex region at the 3′-end, 5′-end or both ends of the strand.

When the sense strand and the antisense strand of the RNAi agent each contain at least one wing modification, the wing modifications may fall on the same end of the duplex region, and have an overlap of one, two or three nucleotides.

When the sense strand and the antisense strand of the RNAi agent each contain at least two wing modifications, the sense strand and the antisense strand can be so aligned that two modifications each from one strand fall on one end of the duplex region, having an overlap of one, two or three nucleotides; two modifications each from one strand fall on the other end of the duplex region, having an overlap of one, two or three nucleotides; two modifications one strand fall on each side of the lead motif, having an overlap of one, two, or three nucleotides in the duplex region.

In one embodiment, the RNAi agent comprises mismatch(es) with the target, within the duplex, or combinations thereof. The mismatch may occur in the overhang region or the duplex region. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In one embodiment, the RNAi agent comprises at least one of the first 1, 2, 3, 4, or 5 base pairs within the duplex regions from the 5′- end of the antisense strand independently selected from the group of: A:U, G:U, I:C, and mismatched pairs, e.g., non-canonical or other than canonical pairings or pairings which include a universal base, to promote the dissociation of the antisense strand at the 5′-end of the duplex.

In one embodiment, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′- end of the antisense strand is an AU base pair. For example, the first base pair within the duplex region from the 5′- end of the antisense strand is an AU base pair.

In another embodiment, the nucleotide at the 3′-end of the sense strand is deoxy-thymine (dT). In another embodiment, the nucleotide at the 3′-end of the antisense strand is deoxy-thymine (dT). In one embodiment, there is a short sequence of deoxy-thymine nucleotides, for example, two dT nucleotides on the 3′-end of the sense or antisense strand.

In one embodiment, the sense strand sequence may be represented by formula (I):

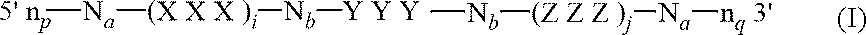

wherein:
i and j are each independently 0 or 1;
p and q are each independently 0-6;
each Nₐ independently represents an oligonucleotide sequence comprising 0-25 modified nucleotides, each sequence comprising at least two differently modified nucleotides;
each N_{b} independently represents an oligonucleotide sequence comprising 0-10 modified nucleotides;
each nₚ and n_{q} independently represent an overhang nucleotide;
wherein Nb and Y do not have the same modification; and
XXX, YYY and ZZZ each independently represent one motif of three identical modifications on three consecutive nucleotides. Preferably YYY is all 2′-F modified nucleotides.

In one embodiment, the Nₐ or N_{b} comprise modifications of alternating pattern.

In one embodiment, the YYY motif occurs at or near the cleavage site of the sense strand. For example, when the RNAi agent has a duplex region of 17-23 nucleotides in length, the YYY motif can occur at or the vicinity of the cleavage site (e.g.: can occur at positions 6, 7, 8, 7, 8, 9, 8, 9, 10, 9, 10, 11, 10, 11,12 or 11, 12, 13) of - the sense strand, the count starting from the 1^{st} nucleotide, from the 5′-end; or optionally, the count starting at the 1^{st} paired nucleotide within the duplex region, from the 5′- end.

In one embodiment, i is 1 and j is 0, or i is 0 and j is 1, or both i and j are 1. The sense strand can therefore be represented by the following formulas:

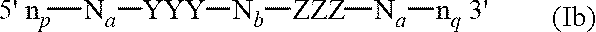

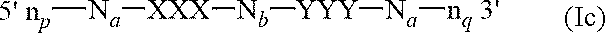

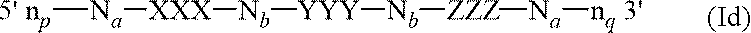

When the sense strand is represented by formula (Ib), N_{b} represents an oligonucleotide sequence comprising 0-10, 0-7, 0-5, 0-4, 0-2 or 0 modified nucleotides.

Each Nₐ independently can represent an oligonucleotide sequence comprising 2-20, 2-15, or 2-10 modified nucleotides.

When the sense strand is represented as formula (Ic), N_{b} represents an oligonucleotide sequence comprising 0-10, 0-7, 0-10, 0-7, 0-5, 0-4, 0-2 or 0 modified nucleotides. Each Nₐ can independently represent an oligonucleotide sequence comprising 2-20, 2-15, or 2-10 modified nucleotides.

When the sense strand is represented as formula (Id), each N_{b} independently represents an oligonucleotide sequence comprising 0-10, 0-7, 0-5, 0-4, 0-2 or 0 modified nucleotides. Preferably, N_{b} is 0, 1, 2, 3, 4, 5 or 6. Each Nₐ can independently represent an oligonucleotide sequence comprising 2-20, 2-15, or 2-10 modified nucleotides.

Each of X, Y and Z may be the same or different from each other.

In other embodiments, i is 0 and j is 0, and the sense strand may be represented by the formula:

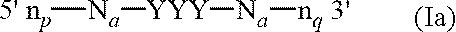

When the sense strand is represented by formula (Ia), each Nₐ independently can represent an oligonucleotide sequence comprising 2-20, 2-15, or 2-10 modified nucleotides.

In one embodiment, the antisense strand sequence of the RNAi may be represented by formula (II):

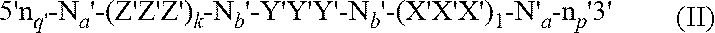

wherein:
k and 1 are each independently 0 or 1;
p′ and q′ are each independently 0-6;
each Nₐ’ independently represents an oligonucleotide sequence comprising 0-25 modified nucleotides, each sequence comprising at least two differently modified nucleotides;
each N_{b}’ independently represents an oligonucleotide sequence comprising 0-10 modified nucleotides; each nₚ’ and n_{q}’ independently represent an overhang nucleotide;
wherein N_{b}’ and Y′ do not have the same modification;
and X′X′X′, Y′Y′Y′ and Z′Z′Z′ each independently represent one motif of three identical modifications on three consecutive nucleotides.

In one embodiment, the Nₐ′ or N_{b}′ comprise modifications of alternating pattern.

The Y′Y′Y′ motif occurs at or near the cleavage site of the antisense strand. For example, when the RNAi agent has a duplex region of 17-23nucleotidein length, the Y′Y′Y′ motif can occur at positions 9, 10, 11;10, 11, 12; 11, 12, 13; 12, 13, 14; or 13, 14, 15 of the antisense strand, with the count starting from the 1^{st} nucleotide, from the 5′-end; or optionally, the count starting at the 1^{st} paired nucleotide within the duplex region, from the 5′- end. Preferably, the Y′Y′Y′ motif occurs at positions 11, 12, 13.

In one embodiment, Y′Y′Y′ motif is all 2′-OMe modified nucleotides.

In one embodiment, k is 1 and 1 is 0, or k is 0 and 1 is 1, or both k and 1 are 1.

The antisense strand can therefore be represented by the following formulas:

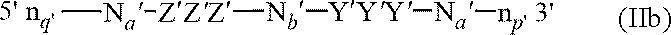

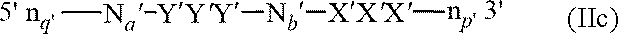

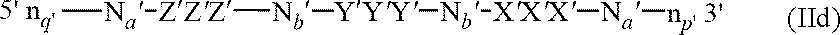

When the antisense strand is represented by formula (IIb), N_{b}′ represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as formula (IIc), N_{b}′ represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides.

When the antisense strand is represented as formula (IId), each N_{b}′ independently represents an oligonucleotide sequence comprising 0-10, 0-7, 0-10, 0-7, 0-5, 0-4, 0-2 or 0 modified nucleotides. Each Nₐ′ independently represents an oligonucleotide sequence comprising 2-20, 2-15, or 2-10 modified nucleotides. Preferably, N_{b} is 0, 1, 2, 3, 4, 5 or 6.

In other embodiments, k is 0 and 1 is 0 and the antisense strand may be represented by the formula:

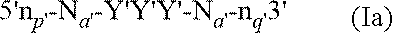

When the antisense strand is represented as formula (IIa), each Nₐ’ independently represents an oligonucleotide sequence comprising 2-20, 2-15, or 2-10 modified nucleotides.

Each of X′, Y′ and Z′ may be the same or different from each other.

Each nucleotide of the sense strand and antisense strand may be independently modified with LNA, HNA, CeNA, 2′-methoxyethyl, 2′-O-methyl, 2′-O-allyl, 2′-C- allyl, 2′-hydroxyl, or 2′-fluoro. For example, each nucleotide of the sense strand and antisense strand is independently modified with 2′-O-methyl or 2′-fluoro. Each X, Y, Z, X′, Y′ and Z′, in particular, may represent a 2′-O-methyl modification or a 2′-fluoro modification.

In one embodiment, the sense strand of the RNAi agent may contain YYY motif occurring at 9, 10 and 11 positions of the strand when the duplex region is 21 nt, the count starting from the 1^{st} nucleotide from the 5′-end, or optionally, the count starting at the 1^{st} paired nucleotide within the duplex region, from the 5′- end; and Y represents 2′-F modification. The sense strand may additionally contain XXX motif or ZZZ motifs as wing modifications at the opposite end of the duplex region; and XXX and ZZZ each independently represents a 2′-OMe modification or 2′-F modification.

In one embodiment the antisense strand may contain Y′Y′Y′ motif occurring at positions 11, 12, 13 of the strand, the count starting from the 1^{st} nucleotide from the 5′-end, or optionally, the count starting at the 1^{st} paired nucleotide within the duplex region, from the 5′- end; and Y′ represents 2′-O-methyl modification. The antisense strand may additionally contain X′X′X′ motif or Z′Z′Z′ motifs as wing modifications at the opposite end of the duplex region; and X′X′X′ and Z′Z′Z′ each independently represents a 2′-OMe modification or 2′-F modification.

The sense strand represented by any one of the above formulas (Ia), (Ib), (Ic), and (Id) forms a duplex with an antisense strand being represented by any one of formulas (IIa), (IIb), (IIc), and (IId), respectively.

Accordingly, the RNAi agents for use in the methods of the disclosure may comprise a sense strand and an antisense strand, each strand having 14 to 30 nucleotides, the RNAi duplex represented by formula (III):

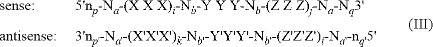

wherein:
i, j, k, and 1 are each independently 0 or 1;
p, p′, q, and q′ are each independently 0-6;
each Nₐ and Nₐ independently represents an oligonucleotide sequence comprising 0-25 modified nucleotides, each sequence comprising at least two differently modified nucleotides;
each N_{b} and N_{b}’ independently represents an oligonucleotide sequence comprising 0-10 modified nucleotides; wherein
each nₚ’, nₚ, n_{q}’, and n_{q}, each of which may or may not be present, independently represents an overhang nucleotide; and
XXX, YYY, ZZZ, X′X′X′, Y′Y′Y′, and Z′Z′Z′ each independently represent one motif of three identical modifications on three consecutive nucleotides.

In one embodiment, i is 0 and j is 0; or i is 1 and j is 0; or i is 0 and j is 1; or both i and j are 0; or both i and j are 1. In another embodiment, k is 0 and 1 is 0; or k is 1 and 1 is 0; k is 0 and 1 is 1; or both k and 1 are 0; or both k and 1 are 1.

Exemplary combinations of the sense strand and antisense strand forming an RNAi duplex include the formulas below:

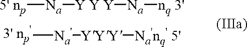

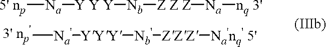

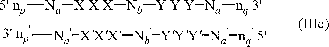

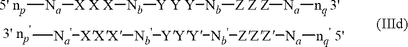

When the RNAi agent is represented by formula (IIIa), each Nₐ independently represents an oligonucleotide sequence comprising 2-20, 2-15, or 2-10 modified nucleotides.

When the RNAi agent is represented by formula (IIIb), each N_{b} independently represents an oligonucleotide sequence comprising 1-10, 1-7, 1-5 or 1-4 modified nucleotides. Each Nₐ independently represents an oligonucleotide sequence comprising 2-20, 2-15, or 2-10 modified nucleotides.

When the RNAi agent is represented as formula (IIIc), each N_{b}, N_{b}′ independently represents an oligonucleotide sequence comprising 0-10, 0-7, 0-10, 0-7, 0-5, 0-4, 0-2 or 0modified nucleotides. Each Nₐ independently represents an oligonucleotide sequence comprising 2-20, 2-15, or 2-10 modified nucleotides.

When the RNAi agent is represented as formula (IIId), each N_{b}, N_{b}′ independently represents an oligonucleotide sequence comprising 0-10, 0-7, 0-10, 0-7, 0-5, 0-4, 0-2 or 0 modified nucleotides. Each Nₐ, Nₐ independently represents an oligonucleotide sequence comprising 2-20, 2-15, or 2-10 modified nucleotides. Each of Nₐ, Nₐ′, N_{b} and N_{b}’ independently comprises modifications of alternating pattern.

In one embodiment, when the RNAi agent is represented by formula (IIId), the Nₐ modifications are 2′-O-methyl or 2′-fluoro modifications. In another embodiment, when the RNAi agent is represented by formula (IIId), the Nₐ modifications are 2′-O-methyl or 2′-fluoro modifications and nₚ′ >0 and at least one nₚ′ is linked to a neighboring nucleotide a via phosphorothioate linkage. In yet another embodiment, when the RNAi agent is represented by formula (IIId), the Nₐ modifications are 2′-O-methyl or 2′-fluoro modifications, nₚ′ >0 and at least one nₚ′ is linked to a neighboring nucleotide via phosphorothioate linkage, and the sense strand is conjugated to one or more C16 (or related) moieties attached through a bivalent or trivalent branched linker (described below). In another embodiment, when the RNAi agent is represented by formula (IIId), the Nₐ modifications are 2′-O-methyl or 2′-fluoro modifications, nₚ′ >0 and at least one nₚ′ is linked to a neighboring nucleotide via phosphorothioate linkage, the sense strand comprises at least one phosphorothioate linkage, and the sense strand is conjugated to one or more lipophilic, e.g., C16 (or related) moieties, optionally attached through a bivalent or trivalent branched linker.

In one embodiment, when the RNAi agent is represented by formula (IIIa), the Nₐ modifications are 2′-O-methyl or 2′-fluoro modifications, nₚ′ >0 and at least one nₚ′ is linked to a neighboring nucleotide via phosphorothioate linkage, the sense strand comprises at least one phosphorothioate linkage, and the sense strand is conjugated to one or more lipophilic, e.g., C16 (or related) moieties attached through a bivalent or trivalent branched linker.

In one embodiment, the RNAi agent is a multimer containing at least two duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, the RNAi agent is a multimer containing three, four, five, six or more duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, two RNAi agents represented by formula (III), (IIIa), (IIIb), (IIIc), and (IIId) are linked to each other at the 5′ end, and one or both of the 3′ ends and are optionally conjugated to to a ligand. Each of the agents can target the same gene or two different genes; or each of the agents can target same gene at two different target sites.

Various publications describe multimeric RNAi agents that can be used in the methods of the disclosure. Such publications include WO2007/091269, WO2010/141511, WO2007/117686, WO2009/014887, and WO2011/031520; and US 7858769, the entire contents of each of which are hereby incorporated herein by reference.

In certain embodiments, the compositions and methods of the disclosure include a vinyl phosphonate (VP) modification of an RNAi agent as described herein. In exemplary embodiments, a vinyl phosphonate of the disclosure has the following structure:

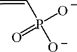

For example, when the phosphate mimic is a 5′-vinyl phosphonate (VP), the 5′-terminal nucleotide can have the following structure,

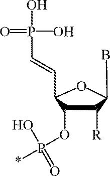

wherein * indicates the location of the bond to 5′-position of the adjacent nucleotide;
R is hydrogen, hydroxy, methoxy, fluoro (e.g., hydroxy or methoxy), or another modification described herein; and
B is a nucleobase or a modified nucleobase, optionally where B is adenine, guanine, cytosine, thymine or uracil.

A vinyl phosphonate of the instant disclosure may be attached to either the antisense or the sense strand of a dsRNA of the disclosure. In certain embodiments, a vinyl phosphonate of the instant disclosure is attached to the antisense strand of a dsRNA, optionally at the 5′ end of the antisense strand of the dsRNA.

Vinyl phosphate modifications are also contemplated for the compositions and methods of the instant disclosure. An exemplary vinyl phosphate structure is:

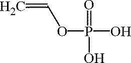

For example, when the phosphate mimic is a 5′-vinyl phosphate, the 5′-terminal nucleotide can have the immediately structure, where the phosphonate group is replaced by a phosphate.

### I. Thermally Destabilizing Modifications

In certain embodiments, a dsRNA molecule can be optimized for RNA interference by incorporating thermally destabilizing modifications in the seed region of the antisense strand. As used herein “seed region” means at positions 2-9 of the 5′-end of the referenced strand. For example, thermally destabilizing modifications can be incorporated in the seed region of the the antisense strand to reduce or inhibit off-target gene silencing.

The term “thermally destabilizing modification(s)” includes modification(s) that would result with a dsRNA with a lower overall melting temperature (Tₘ) than the Tₘ of the dsRNA without having such modification(s). For example, the thermally destabilizing modification(s) can decrease the Tₘ of the dsRNA by 1 - 4° C., such as one, two, three or four degrees Celcius. And, the term “thermally destabilizing nucleotide” refers to a nucleotide containing one or more thermally destabilizing modifications.

It has been discovered that dsRNAs with an antisense strand comprising at least one thermally destabilizing modification of the duplex within the first 9 nucleotide positions, counting from the 5′ end, of the antisense strand have reduced off-target gene silencing activity. Accordingly, in some embodiments, the antisense strand comprises at least one (e.g., one, two, three, four, five or more) thermally destabilizing modification of the duplex within the first 9 nucleotide positions of the 5′ region of the antisense strand. In some embodiments, one or more thermally destabilizing modification(s) of the duplex is/are located in positions 2-9, or preferably positions 4-8, from the 5′-end of the antisense strand. In some further embodiments, the thermally destabilizing modification(s) of the duplex is/are located at position 6, 7 or 8 from the 5′-end of the antisense strand. In still some further embodiments, the thermally destabilizing modification of the duplex is located at position 7 from the 5′-end of the antisense strand. In some embodiments, the thermally destabilizing modification of the duplex is located at position 2, 3, 4, 5 or 9 from the 5′-end of the antisense strand.

The thermally destabilizing modifications can include, but are not limited to, abasic modification; mismatch with the opposing nucleotide in the opposing strand; and sugar modification such as 2′-deoxy modification or acyclic nucleotide, e.g., unlocked nucleic acids (UNA) or glycol nucleic acid (GNA).

Exemplified abasic modifications include, but are not limited to the following:

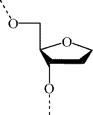

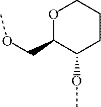

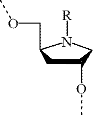

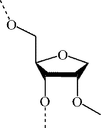

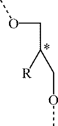

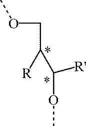

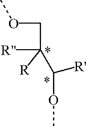

Wherein R = H, Me, Et or OMe; R′ = H, Me, Et or OMe; R″ = H, Me, Et or OMe

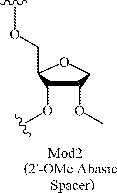

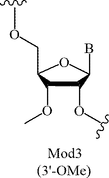

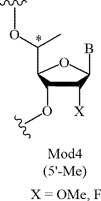

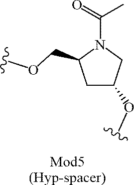

wherein B is a modified or unmodified nucleobase.

Exemplified sugar modifications include, but are not limited to the following:

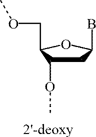

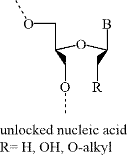

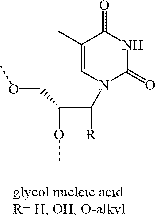

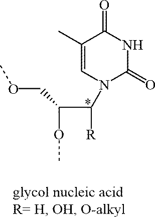

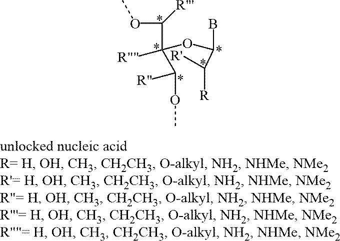

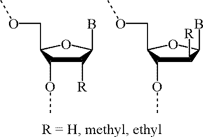

wherein B is a modified or unmodified nucleobase.

In some embodiments the thermally destabilizing modification of the duplex is selected from the group consisting of:

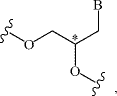

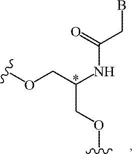

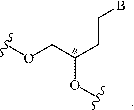

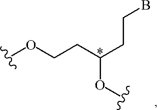

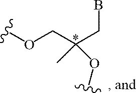

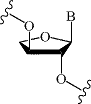

wherein B is a modified or unmodified nucleobase and the asterisk on each structure represents either R, S or racemic.

The term “acyclic nucleotide” refers to any nucleotide having an acyclic ribose sugar, for example, where any of bonds between the ribose carbons (e.g., C1′-C2’, C2′-C3’, C3′-C4’, C4′-O4’, or C1′-O4’) is absent or at least one of ribose carbons or oxygen (e.g., C1′, C2′, C3′, C4′ or O4′) are independently or in combination absent from the nucleotide. In some embodiments, acyclic nucleotide

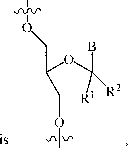

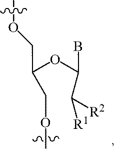

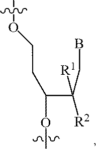

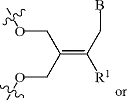

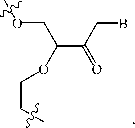

wherein B is a modified or unmodified nucleobase, R¹ and R² independently are H, halogen, OR₃, or alkyl; and R₃ is H, alkyl, cycloalkyl, aryl, aralkyl, heteroaryl or sugar). The term “UNA” refers to unlocked acyclic nucleic acid, wherein any of the bonds of the sugar has been removed, forming an unlocked “sugar” residue. In one example, UNA also encompasses monomers with bonds between C1′-C4’ being removed (i.e. the covalent carbon-oxygen-carbon bond between the C1′ and C4′ carbons). In another example, the C2′-C3’ bond (i.e. the covalent carbon-carbon bond between the C2′ and C3′ carbons) of the sugar is removed (see Mikhailov et. al., Tetrahedron Letters, 26 (17): 2059 (1985); and Fluiter et al., Mol. Biosyst., 10: 1039 (2009), which are hereby incorporated by reference in their entirety). The acyclic derivative provides greater backbone flexibility without affecting the Watson-Crick pairings. The acyclic nucleotide can be linked via 2′-5′ or 3′-5′ linkage.

The term ‘GNA’ refers to glycol nucleic acid which is a polymer similar to DNA or RNA but differing in the composition of its “backbone” in that is composed of repeating glycerol units linked by phosphodiester bonds:

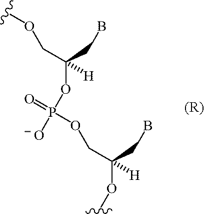

The thermally destabilizing modification of the duplex can be mismatches (i.e., noncomplementary base pairs) between the thermally destabilizing nucleotide and the opposing nucleotide in the opposite strand within the dsRNA duplex. Exemplary mismatch base pairs include G:G, G:A, G:U, G:T, A:A, A:C, C:C, C:U, C:T, U:U, T:T, U:T, or a combination thereof. Other mismatch base pairings known in the art are also amenable to the present invention. A mismatch can occur between nucleotides that are either naturally occurring nucleotides or modified nucleotides, i.e., the mismatch base pairing can occur between the nucleobases from respective nucleotides independent of the modifications on the ribose sugars of the nucleotides. In certain embodiments, the dsRNA molecule contains at least one nucleobase in the mismatch pairing that is a 2′-deoxy nucleobase; e.g., the 2′-deoxy nucleobase is in the sense strand.

In some embodiments, the thermally destabilizing modification of the duplex in the seed region of the antisense strand includes nucleotides with impaired W-C H-bonding to complementary base on the target mRNA, such as:

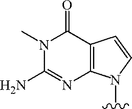

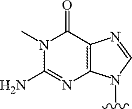

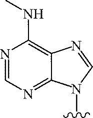

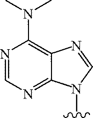

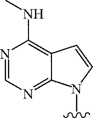

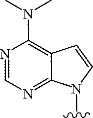

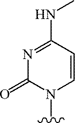

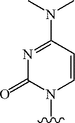

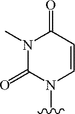

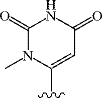

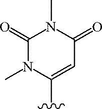

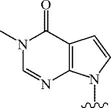

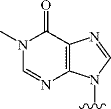

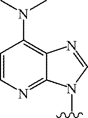

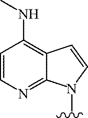

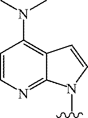

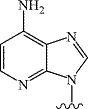

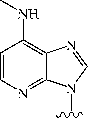

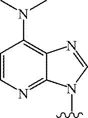

More examples of abasic nucleotide, acyclic nucleotide modifications (including UNA and GNA), and mismatch modifications have been described in detail in WO 2011/133876, which is herein incorporated by reference in its entirety.

The thermally destabilizing modifications may also include universal base with reduced or abolished capability to form hydrogen bonds with the opposing bases, and phosphate modifications.

In some embodiments, the thermally destabilizing modification of the duplex includes nucleotides with non-canonical bases such as, but not limited to, nucleobase modifications with impaired or completely abolished capability to form hydrogen bonds with bases in the opposite strand. These nucleobase modifications have been evaluated for destabilization of the central region of the dsRNA duplex as described in WO 2010/0011895, which is herein incorporated by reference in its entirety. Exemplary nucleobase modifications are:

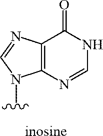

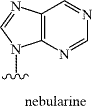

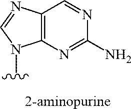

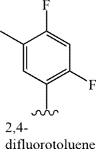

]

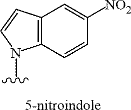

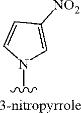

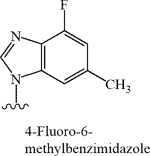

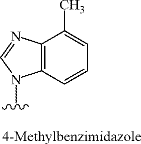

In some embodiments, the thermally destabilizing modification of the duplex in the seed region of the antisense strand includes one or more α-nucleotide complementary to the base on the target mRNA, such as:

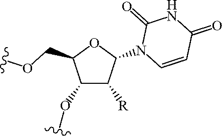

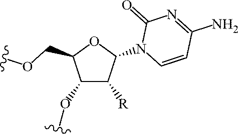

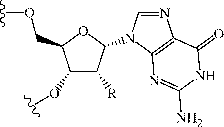

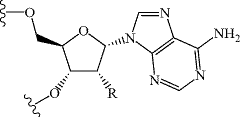

wherein R is H, OH, OCH₃, F, NH₂, NHMe, NMe₂ or O-alkyl.

Exemplary phosphate modifications known to decrease the thermal stability of dsRNA duplexes compared to natural phosphodiester linkages are:

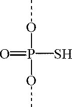

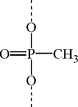

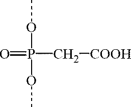

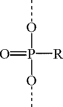

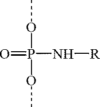

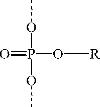

R = alkyl

The alkyl for the R group can be a C₁-C₆alkyl. Specific alkyls for the R group include, but are not limited to methyl, ethyl, propyl, isopropyl, butyl, pentyl and hexyl.

As the skilled artisan will recognize, in view of the functional role of nucleobases is defining specificity of an RNAi agent of the disclosure, while nucleobase modifications can be performed in the various manners as described herein, e.g., to introduce destabilizing modifications into an RNAi agent of the disclosure, e.g., for purpose of enhancing on-target effect relative to off-target effect, the range of modifications available and, in general, present upon RNAi agents of the disclosure tends to be much greater for non-nucleobase modifications, e.g., modifications to sugar groups or phosphate backbones of polyribonucleotides. Such modifications are described in greater detail in other sections of the instant disclosure and are expressly contemplated for RNAi agents of the disclosure, either possessing native nucleobases or modified nucleobases as described above or elsewhere herein.

In addition to the antisense strand comprising a thermally destabilizing modification, the dsRNA can also comprise one or more stabilizing modifications. For example, the dsRNA can comprise at least two (e.g., two, three, four, five, six, seven, eight, nine, ten or more) stabilizing modifications. Without limitations, the stabilizing modifications all can be present in one strand. In some embodiments, both the sense and the antisense strands comprise at least two stabilizing modifications. The stabilizing modification can occur on any nucleotide of the sense strand or antisense strand. For instance, the stabilizing modification can occur on every nucleotide on the sense strand or antisense strand; each stabilizing modification can occur in an alternating pattern on the sense strand or antisense strand; or the sense strand or antisense strand comprises both stabilizing modification in an alternating pattern. The alternating pattern of the stabilizing modifications on the sense strand may be the same or different from the antisense strand, and the alternating pattern of the stabilizing modifications on the sense strand can have a shift relative to the alternating pattern of the stabilizing modifications on the antisense strand.

In some embodiments, the antisense strand comprises at least two (e.g., two, three, four, five, six, seven, eight, nine, ten or more) stabilizing modifications. Without limitations, a stabilizing modification in the antisense strand can be present at any positions. In some embodiments, the antisense comprises stabilizing modifications at positions 2, 6, 8, 9, 14, and 16 from the 5′-end. In some other embodiments, the antisense comprises stabilizing modifications at positions 2, 6, 14, and 16 from the 5′-end. In still some other embodiments, the antisense comprises stabilizing modifications at positions 2, 14, and 16 from the 5′-end.

In some embodiments, the antisense strand comprises at least one stabilizing modification adjacent to the destabilizing modification. For example, the stabilizing modification can be the nucleotide at the 5′-end or the 3′-end of the destabilizing modification, i.e., at position -1 or +1 from the position of the destabilizing modification. In some embodiments, the antisense strand comprises a stabilizing modification at each of the 5′-end and the 3′-end of the destabilizing modification, i.e., positions -1 and +1 from the position of the destabilizing modification.

In some embodiments, the antisense strand comprises at least two stabilizing modifications at the 3′-end of the destabilizing modification, i.e., at positions +1 and +2 from the position of the destabilizing modification.

In some embodiments, the sense strand comprises at least two (e.g., two, three, four, five, six, seven, eight, nine, ten or more) stabilizing modifications. Without limitations, a stabilizing modification in the sense strand can be present at any positions. In some embodiments, the sense strand comprises stabilizing modifications at positions 7, 10, and 11 from the 5′-end. In some other embodiments, the sense strand comprises stabilizing modifications at positions 7, 9, 10, and 11 from the 5′-end. In some embodiments, the sense strand comprises stabilizing modifications at positions opposite or complimentary to positions 11, 12, and 15 of the antisense strand, counting from the 5′-end of the antisense strand. In some other embodiments, the sense strand comprises stabilizing modifications at positions opposite or complimentary to positions 11, 12, 13, and 15 of the antisense strand, counting from the 5′-end of the antisense strand. In some embodiments, the sense strand comprises a block of two, three, or four stabilizing modifications.

In some embodiments, the sense strand does not comprise a stabilizing modification in position opposite or complimentary to the thermally destabilizing modification of the duplex in the antisense strand.

Exemplary thermally stabilizing modifications include, but are not limited to, 2′-fluoro modifications. Other thermally stabilizing modifications include, but are not limited to, LNA.

In some embodiments, the dsRNA of the disclosure comprises at least four (e.g., four, five, six, seven, eight, nine, ten, or more) 2′-fluoro nucleotides. Without limitations, the 2′-fluoro nucleotides all can be present in one strand. In some embodiments, both the sense and the antisense strands comprise at least two 2′-fluoro nucleotides. The 2′-fluoro modification can occur on any nucleotide of the sense strand or antisense strand. For instance, the 2′-fluoro modification can occur on every nucleotide on the sense strand or antisense strand; each 2′-fluoro modification can occur in an alternating pattern on the sense strand or antisense strand; or the sense strand or antisense strand comprises both 2′-fluoro modifications in an alternating pattern. The alternating pattern of the 2′-fluoro modifications on the sense strand may be the same or different from the antisense strand, and the alternating pattern of the 2′-fluoro modifications on the sense strand can have a shift relative to the alternating pattern of the 2′-fluoro modifications on the antisense strand.

In some embodiments, the antisense strand comprises at least two (e.g., two, three, four, five, six, seven, eight, nine, ten, or more) 2′-fluoro nucleotides. Without limitations, a 2′-fluoro modification in the antisense strand can be present at any positions. In some embodiments, the antisense comprises 2′-fluoro nucleotides at positions 2, 6, 8, 9, 14, and 16 from the 5′-end. In some other embodiments, the antisense comprises 2′-fluoro nucleotides at positions 2, 6, 14, and 16 from the 5′-end. In still some other embodiments, the antisense comprises 2′-fluoro nucleotides at positions 2, 14, and 16 from the 5′-end.

In some embodiments, the antisense strand comprises at least one 2′-fluoro nucleotide adjacent to the destabilizing modification. For example, the 2′-fluoro nucleotide can be the nucleotide at the 5′-end or the 3′-end of the destabilizing modification, i. e., at position -1 or +1 from the position of the destabilizing modification. In some embodiments, the antisense strand comprises a 2′-fluoro nucleotide at each of the 5′-end and the 3′-end of the destabilizing modification, i.e., positions -1 and +1 from the position of the destabilizing modification.

In some embodiments, the antisense strand comprises at least two 2′-fluoro nucleotides at the 3′-end of the destabilizing modification, i.e., at positions +1 and +2 from the position of the destabilizing modification.

In some embodiments, the sense strand comprises at least two (e.g., two, three, four, five, six, seven, eight, nine, ten or more) 2′-fluoro nucleotides. Without limitations, a 2′-fluoro modification in the sense strand can be present at any positions. In some embodiments, the antisense comprises 2′-fluoro nucleotides at positions 7, 10, and 11 from the 5′-end. In some other embodiments, the sense strand comprises 2′-fluoro nucleotides at positions 7, 9, 10, and 11 from the 5′-end. In some embodiments, the sense strand comprises 2′-fluoro nucleotides at positions opposite or complimentary to positions 11, 12, and 15 of the antisense strand, counting from the 5′-end of the antisense strand. In some other embodiments, the sense strand comprises 2′-fluoro nucleotides at positions opposite or complimentary to positions 11, 12, 13, and 15 of the antisense strand, counting from the 5′-end of the antisense strand. In some embodiments, the sense strand comprises a block of two, three or four 2′-fluoro nucleotides.

In some embodiments, the sense strand does not comprise a 2′-fluoro nucleotide in position opposite or complimentary to the thermally destabilizing modification of the duplex in the antisense strand.

In some embodiments, the dsRNA molecule of the disclosure comprises a 21 nucleotides (nt) sense strand and a 23 nucleotides (nt) antisense, wherein the antisense strand contains at least one thermally destabilizing nucleotide, where the at least one thermally destabilizing nucleotide occurs in the seed region of the antisense strand (i.e., at position 2-9 of the 5′-end of the antisense strand), wherein one end of the dsRNA is blunt, while the other end is comprises a 2 nt overhang, and wherein the dsRNA optionally further has at least one (e.g., one, two, three, four, five, six or all seven) of the following characteristics: (i) the antisense comprises 2, 3, 4, 5 or 6 2′-fluoro modifications; (ii) the antisense comprises 1, 2, 3, 4 or 5 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3, 4 or 5 phosphorothioate internucleotide linkages; (vi) the dsRNA comprises at least four 2′-fluoro modifications; and (vii) the dsRNA comprises a blunt end at 5′-end of the antisense strand. Preferably, the 2 nt overhang is at the 3′-end of the antisense.

In some embodiments, the dsRNA molecule of the disclosure comprising a sense and antisense strands, wherein: the sense strand is 25-30 nucleotide residues in length, wherein starting from the 5′ terminal nucleotide (position 1), positions 1 to 23 of said sense strand comprise at least 8 ribonucleotides; antisense strand is 36-66 nucleotide residues in length and, starting from the 3′ terminal nucleotide, at least 8 ribonucleotides in the positions paired with positions 1- 23 of sense strand to form a duplex; wherein at least the 3 ′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when said double stranded nucleic acid is introduced into a mammalian cell; and wherein the antisense strand contains at least one thermally destabilizing nucleotide, where at least one thermally destabilizing nucleotide is in the seed region of the antisense strand (i.e. at position 2-9 of the 5′-end of the antisense strand). For example, the thermally destabilizing nucleotide occurs between positions opposite or complimentary to positions 14-17 of the 5′-end of the sense strand, and wherein the dsRNA optionally further has at least one (e.g., one, two, three, four, five, six or all seven) of the following characteristics: (i) the antisense comprises 2, 3, 4, 5, or 6 2′-fluoro modifications; (ii) the antisense comprises 1, 2, 3, 4, or 5 phosphorothioate internucleotide linkages; (iii) the sense strand is conjugated with a ligand; (iv) the sense strand comprises 2, 3, 4, or 5 2′-fluoro modifications; (v) the sense strand comprises 1, 2, 3, 4, or 5 phosphorothioate internucleotide linkages; and (vi) the dsRNA comprises at least four 2′-fluoro modifications; and (vii) the dsRNA comprises a duplex region of 12-30 nucleotide pairs in length.

In some embodiments, the dsRNA molecule of the disclosure comprises a sense and antisense strands, wherein said dsRNA molecule comprises a sense strand having a length which is at least 25 and at most 29 nucleotides and an antisense strand having a length which is at most 30 nucleotides with the sense strand comprises a modified nucleotide that is susceptible to enzymatic degradation at position 11 from the 5′end, wherein the 3′ end of said sense strand and the 5′ end of said antisense strand form a blunt end and said antisense strand is 1-4 nucleotides longer at its 3′ end than the sense strand, wherein the duplex region which is at least 25 nucleotides in length, and said antisense strand is sufficiently complementary to a target mRNA along at least 19 nt of said antisense strand length to reduce target gene expression when said dsRNA molecule is introduced into a mammalian cell, and wherein dicer cleavage of said dsRNA preferentially results in an siRNA comprising said 3′ end of said antisense strand, thereby reducing expression of the target gene in the mammal, wherein the antisense strand contains at least one thermally destabilizing nucleotide, where the at least one thermally destabilizing nucleotide is in the seed region of the antisense strand (i.e. at position 2-9 of the 5′-end of the antisense strand), and wherein the dsRNA optionally further has at least one (e.g., one, two, three, four, five, six or all seven) of the following characteristics: (i) the antisense comprises 2, 3, 4, 5, or 6 2′-fluoro modifications; (ii) the antisense comprises 1, 2, 3, 4, or 5 phosphorothioate internucleotide linkages; (iii) the sense strand is conjugated with a ligand; (iv) the sense strand comprises 2, 3, 4, or 5 2′-fluoro modifications; (v) the sense strand comprises 1, 2, 3, 4, or 5 phosphorothioate internucleotide linkages; and (vi) the dsRNA comprises at least four 2′-fluoro modifications; and (vii) the dsRNA has a duplex region of 12-29 nucleotide pairs in length.

In some embodiments, every nucleotide in the sense strand and antisense strand of the dsRNA molecule may be modified. Each nucleotide may be modified with the same or different modification which can include one or more alteration of one or both of the non-linking phosphate oxygens or of one or more of the linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases, the modification will occur at all of the subject positions in the nucleic acid but in many cases it will not. By way of example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of an RNA. E.g., a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′ end or ends can be phosphorylated.

It may be possible, e.g., to enhance stability, to include particular bases in overhangs, or to include modified nucleotides or nucleotide surrogates, in single strand overhangs, e.g., in a 5′ or 3′ overhang, or in both. E.g., it can be desirable to include purine nucleotides in overhangs. In some embodiments all or some of the bases in a 3′ or 5′ overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, each residue of the sense strand and antisense strand is independently modified with LNA, HNA, CeNA, 2′-methoxyethyl, 2′- O-methyl, 2′-O-allyl, 2′-C- allyl, 2′-deoxy, or 2′-fluoro. The strands can contain more than one modification. In some embodiments, each residue of the sense strand and antisense strand is independently modified with 2′-O-methyl or 2′-fluoro. It is to be understood that these modifications are in addition to the at least one thermally destabilizing modification of the duplex present in the antisense strand.

At least two different modifications are typically present on the sense strand and antisense strand. Those two modifications may be the 2′-deoxy, 2′-O-methyl or 2′-fluoro modifications, acyclic nucleotides or others. In some embodiments, the sense strand and antisense strand each comprises two differently modified nucleotides selected from 2′-O-methyl or 2′-deoxy. In some embodiments, each residue of the sense strand and antisense strand is independently modified with 2′-O-methyl nucleotide, 2′-deoxy nucleotide, 2′-deoxy-2′-fluoro nucleotide, 2′-O-N-methylacetamido (2′-O-NMA) nucleotide, a 2′-O-dimethylaminoethoxyethyl (2′-O-DMAEOE) nucleotide, 2′-O-aminopropyl (2′-O-AP) nucleotide, or 2′-ara-F nucleotide. Again, it is to be understood that these modifications are in addition to the at least one thermally destabilizing modification of the duplex present in the antisense strand.

In some embodiments, the dsRNA molecule of the disclosure comprises modifications of an alternating pattern, particular in the B1, B2, B3, B1′, B2′, B3′, B4′ regions. The term “alternating motif” or “alternative pattern” as used herein refers to a motif having one or more modifications, each modification occurring on alternating nucleotides of one strand. The alternating nucleotide may refer to one per every other nucleotide or one per every three nucleotides, or a similar pattern. For example, if A, B and C each represent one type of modification to the nucleotide, the alternating motif can be

"ABABABABABAB...," "AABBAABBAABB...," "AABAABAABAA
B...,""AAABAAABAAAB...," "AAABBBAAABBB...," or "AB
CABCABCABC...," etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as

"ABABAB... ", "ACACAC..." "BDBDBD... " or "CDCDCD.
..," etc.

In some embodiments, the dsRNA molecule of the disclosure comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′-3′ of the strand and the alternating motif in the antisense strand may start with “BABABA” from 3′-5′of the strand within the duplex region. As another example, the alternating motif in the sense strand may start with “AABBAABB” from 5′-3′ of the strand and the alternating motif in the antisense strand may start with “BBAABBAA” from 3′-5′of the strand within the duplex region, so that there is a complete or partial shift of the modification patterns between the sense strand and the antisense strand.

The dsRNA molecule of the disclosure may further comprise at least one phosphorothioate or methylphosphonate internucleotide linkage. The phosphorothioate or methylphosphonate internucleotide linkage modification may occur on any nucleotide of the sense strand or antisense strand or both in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand comprises both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand.

In some embodiments, the dsRNA molecule comprises the phosphorothioate or methylphosphonate internucleotide linkage modification in the overhang region. For example, the overhang region comprises two nucleotides having a phosphorothioate or methylphosphonate internucleotide linkage between the two nucleotides. Internucleotide linkage modifications also may be made to link the overhang nucleotides with the terminal paired nucleotides within duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. Preferably, these terminal three nucleotides may be at the 3′-end of the antisense strand.

In some embodiments, the sense strand of the dsRNA molecule comprises 1-10 blocks of two to ten phosphorothioate or methylphosphonate internucleotide linkages separated by 1, 2, 3, 4, 5, 6, 7, 8, 9, 10, 11, 12, 13, 14, 15, or 16 phosphate internucleotide linkages, wherein one of the phosphorothioate or methylphosphonate internucleotide linkages is placed at any position in the oligonucleotide sequence and the said sense strand is paired with an antisense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of two phosphorothioate or methylphosphonate internucleotide linkages separated by 1, 2, 3, 4, 5, 6, 7, 8, 9, 10, 11, 12, 13, 14, 15, 16, 17, or 18 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of three phosphorothioate or methylphosphonate internucleotide linkages separated by 1, 2, 3, 4, 5, 6, 7, 8, 9, 10, 11, 12, 13, 14, 15, or 16 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of four phosphorothioate or methylphosphonate internucleotide linkages separated by 1, 2, 3, 4, 5, 6, 7, 8, 9, 10, 11, 12, 13, or 14 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of five phosphorothioate or methylphosphonate internucleotide linkages separated by 1, 2, 3, 4, 5, 6, 7, 8, 9, 10, 11, or 12 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of six phosphorothioate or methylphosphonate internucleotide linkages separated by 1, 2, 3, 4, 5, 6, 7, 8, 9, or 10 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of seven phosphorothioate or methylphosphonate internucleotide linkages separated by 1, 2, 3, 4, 5, 6, 7, or 8 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of eight phosphorothioate or methylphosphonate internucleotide linkages separated by 1, 2, 3, 4, 5, or 6 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the antisense strand of the dsRNA molecule comprises two blocks of nine phosphorothioate or methylphosphonate internucleotide linkages separated by 1, 2, 3, or 4 phosphate internucleotide linkages, wherein one of the phosphorothioate or methylphosphonate internucleotide linkages is placed at any position in the oligonucleotide sequence and the said antisense strand is paired with a sense strand comprising any combination of phosphorothioate, methylphosphonate and phosphate internucleotide linkages or an antisense strand comprising either phosphorothioate or methylphosphonate or phosphate linkage.

In some embodiments, the dsRNA molecule of the disclosure further comprises one or more phosphorothioate or methylphosphonate internucleotide linkage modification within 1-10 of the termini position(s) of the sense or antisense strand. For example, at least 2, 3, 4, 5, 6, 7, 8, 9, or 10 nucleotides may be linked through phosphorothioate or methylphosphonate internucleotide linkage at one end or both ends of the sense or antisense strand.

In some embodiments, the dsRNA molecule of the disclosure further comprises one or more phosphorothioate or methylphosphonate internucleotide linkage modification within 1-10 of the internal region of the duplex of each of the sense or antisense strand. For example, at least 2, 3, 4, 5, 6, 7, 8, 9, or 10 nucleotides may be linked through phosphorothioate methylphosphonate internucleotide linkage at position 8-16 of the duplex region counting from the 5′-end of the sense strand; the dsRNA molecule can optionally further comprise one or more phosphorothioate or methylphosphonate internucleotide linkage modification within 1-10 of the termini position(s).

In some embodiments, the dsRNA molecule of the disclosure further comprises one to five phosphorothioate or methylphosphonate internucleotide linkage modification(s) within position 1-5 and one to five phosphorothioate or methylphosphonate internucleotide linkage modification(s) within position 18-23 of the sense strand (counting from the 5′-end), and one to five phosphorothioate or methylphosphonate internucleotide linkage modification at positions 1 and 2 and one to five within positions 18-23 of the antisense strand (counting from the 5′-end).

In some embodiments, the dsRNA molecule of the disclosure further comprises one phosphorothioate internucleotide linkage modification within position 1-5 and one phosphorothioate or methylphosphonate internucleotide linkage modification within position 18-23 of the sense strand (counting from the 5′-end), and one phosphorothioate internucleotide linkage modification at positions 1 and 2 and two phosphorothioate or methylphosphonate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and two phosphorothioate internucleotide linkage modifications within position 18-23 of the sense strand (counting from the 5′-end), and one phosphorothioate internucleotide linkage modification at positions 1 and 2 and one phosphorothioate internucleotide linkage modification within positions 18-23 of the antisense strand (counting from the 5′-end).

In some embodiments, the dsRNA molecule of the disclosure further comprises one phosphorothioate internucleotide linkage modification within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA molecule of the disclosure further comprises one phosphorothioate internucleotide linkage modification within position 1-5 and one within position 18-23 of the sense strand (counting from the 5′-end), and two phosphorothioate internucleotide linkage modification at positions 1 and 2 and one phosphorothioate internucleotide linkage modification within positions 18-23 of the antisense strand (counting from the 5′-end).

In some embodiments, the dsRNA molecule of the disclosure further comprises one phosphorothioate internucleotide linkage modification within position 1-5 (counting from the 5′-end) of the sense strand, and two phosphorothioate internucleotide linkage modifications at positions 1 and 2 and one phosphorothioate internucleotide linkage modification within positions 18-23 of the antisense strand (counting from the 5′-end).

In some embodiments, the dsRNA molecule of the disclosure further comprises two phosphorothioate internucleotide linkage modifications within position 1-5 (counting from the 5′-end) of the sense strand, and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and one within position 18-23 of the sense strand (counting from the 5′-end), and two phosphorothioate internucleotide linkage modifications at positions 1 and 2 and one phosphorothioate internucleotide linkage modification within positions 18-23 of the antisense strand (counting from the 5′-end).

In some embodiments, the dsRNA molecule of the disclosure further comprises two phosphorothioate internucleotide linkage modifications within position 1-5 and one phosphorothioate internucleotide linkage modification within position 18-23 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications within position 1-5 and one phosphorothioate internucleotide linkage modification within position 18-23 of the sense strand (counting from the 5′-end), and one phosphorothioate internucleotide linkage modification at positions 1 and 2 and two phosphorothioate internucleotide linkage modifications within positions 18-23 of the antisense strand (counting from the 5′-end).

In some embodiments, the dsRNA molecule of the disclosure further comprises two phosphorothioate internucleotide linkage modifications at position 1 and 2, and two phosphorothioate internucleotide linkage modifications at position 20 and 21 of the sense strand (counting from the 5′-end), and one phosphorothioate internucleotide linkage modification at positions 1 and one at position 21 of the antisense strand (counting from the 5′-end).

In some embodiments, the dsRNA molecule of the disclosure further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 20 and 21 the antisense strand (counting from the 5′-end).

In some embodiments, the dsRNA molecule of the disclosure further comprises two phosphorothioate internucleotide linkage modifications at position 1 and 2, and two phosphorothioate internucleotide linkage modifications at position 21 and 22 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA molecule of the disclosure further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 21 and 22 the antisense strand (counting from the 5′-end).

In some embodiments, the dsRNA molecule of the disclosure further comprises two phosphorothioate internucleotide linkage modifications at position 1 and 2, and two phosphorothioate internucleotide linkage modifications at position 22 and 23 of the sense strand (counting from the 5′-end), and one phosphorothioate internucleotide linkage modification at positions 1 and one phosphorothioate internucleotide linkage modification at position 21 of the antisense strand (counting from the 5′-end).

In some embodiments, the dsRNA molecule of the disclosure further comprises one phosphorothioate internucleotide linkage modification at position 1, and one phosphorothioate internucleotide linkage modification at position 21 of the sense strand (counting from the 5′-end), and two phosphorothioate internucleotide linkage modifications at positions 1 and 2 and two phosphorothioate internucleotide linkage modifications at positions 23 and 23 the antisense strand (counting from the 5′-end).

In some embodiments, compound of the disclosure comprises a pattern of backbone chiral centers. In some embodiments, a common pattern of backbone chiral centers comprises at least 5 internucleotidic linkages in the Sp configuration. In some embodiments, a common pattern of backbone chiral centers comprises at least 6 internucleotidic linkages in the Sp configuration. In some embodiments, a common pattern of backbone chiral centers comprises at least 7 internucleotidic linkages in the Sp configuration. In some embodiments, a common pattern of backbone chiral centers comprises at least 8 internucleotidic linkages in the Sp configuration. In some embodiments, a common pattern of backbone chiral centers comprises at least 9 internucleotidic linkages in the Sp configuration. In some embodiments, a common pattern of backbone chiral centers comprises at least 10 internucleotidic linkages in the Sp configuration. In some embodiments, a common pattern of backbone chiral centers comprises at least 11 internucleotidic linkages in the Sp configuration. In some embodiments, a common pattern of backbone chiral centers comprises at least 12 internucleotidic linkages in the Sp configuration. In some embodiments, a common pattern of backbone chiral centers comprises at least 13 internucleotidic linkages in the Sp configuration. In some embodiments, a common pattern of backbone chiral centers comprises at least 14 internucleotidic linkages in the Sp configuration. In some embodiments, a common pattern of backbone chiral centers comprises at least 15 internucleotidic linkages in the Sp configuration. In some embodiments, a common pattern of backbone chiral centers comprises at least 16 internucleotidic linkages in the Sp configuration. In some embodiments, a common pattern of backbone chiral centers comprises at least 17 internucleotidic linkages in the Sp configuration. In some embodiments, a common pattern of backbone chiral centers comprises at least 18 internucleotidic linkages in the Sp configuration. In some embodiments, a common pattern of backbone chiral centers comprises at least 19 internucleotidic linkages in the Sp configuration. In some embodiments, a common pattern of backbone chiral centers comprises no more than 8 internucleotidic linkages in the Rp configuration. In some embodiments, a common pattern of backbone chiral centers comprises no more than 7 internucleotidic linkages in the Rp configuration. In some embodiments, a common pattern of backbone chiral centers comprises no more than 6 internucleotidic linkages in the Rp configuration. In some embodiments, a common pattern of backbone chiral centers comprises no more than 5 internucleotidic linkages in the Rp configuration. In some embodiments, a common pattern of backbone chiral centers comprises no more than 4 internucleotidic linkages in the Rp configuration. In some embodiments, a common pattern of backbone chiral centers comprises no more than 3 internucleotidic linkages in the Rp configuration. In some embodiments, a common pattern of backbone chiral centers comprises no more than 2 internucleotidic linkages in the Rp configuration. In some embodiments, a common pattern of backbone chiral centers comprises no more than 1 internucleotidic linkages in the Rp configuration. In some embodiments, a common pattern of backbone chiral centers comprises no more than 8 internucleotidic linkages which are not chiral (as a non-limiting example, a phosphodiester). In some embodiments, a common pattern of backbone chiral centers comprises no more than 7 internucleotidic linkages which are not chiral. In some embodiments, a common pattern of backbone chiral centers comprises no more than 6 internucleotidic linkages which are not chiral. In some embodiments, a common pattern of backbone chiral centers comprises no more than 5 internucleotidic linkages which are not chiral. In some embodiments, a common pattern of backbone chiral centers comprises no more than 4 internucleotidic linkages which are not chiral. In some embodiments, a common pattern of backbone chiral centers comprises no more than 3 internucleotidic linkages which are not chiral. In some embodiments, a common pattern of backbone chiral centers comprises no more than 2 internucleotidic linkages which are not chiral. In some embodiments, a common pattern of backbone chiral centers comprises no more than 1 internucleotidic linkages which are not chiral. In some embodiments, a common pattern of backbone chiral centers comprises at least 10 internucleotidic linkages in the Sp configuration, and no more than 8 internucleotidic linkages which are not chiral. In some embodiments, a common pattern of backbone chiral centers comprises at least 11 internucleotidic linkages in the Sp configuration, and no more than 7 internucleotidic linkages which are not chiral. In some embodiments, a common pattern of backbone chiral centers comprises at least 12 internucleotidic linkages in the Sp configuration, and no more than 6 internucleotidic linkages which are not chiral. In some embodiments, a common pattern of backbone chiral centers comprises at least 13 internucleotidic linkages in the Sp configuration, and no more than 6 internucleotidic linkages which are not chiral. In some embodiments, a common pattern of backbone chiral centers comprises at least 14 internucleotidic linkages in the Sp configuration, and no more than 5 internucleotidic linkages which are not chiral. In some embodiments, a common pattern of backbone chiral centers comprises at least 15 internucleotidic linkages in the Sp configuration, and no more than 4 internucleotidic linkages which are not chiral. In some embodiments, the internucleotidic linkages in the Sp configuration are optionally contiguous or not contiguous. In some embodiments, the internucleotidic linkages in the Rp configuration are optionally contiguous or not contiguous. In some embodiments, the internucleotidic linkages which are not chiral are optionally contiguous or not contiguous.

In some embodiments, compound of the disclosure comprises a block is a stereochemistry block. In some embodiments, a block is an Rp block in that each internucleotidic linkage of the block is Rp. In some embodiments, a 5′-block is an Rp block. In some embodiments, a 3′-block is an Rp block. In some embodiments, a block is an Sp block in that each internucleotidic linkage of the block is Sp. In some embodiments, a 5′-block is an Sp block. In some embodiments, a 3′-block is an Sp block. In some embodiments, provided oligonucleotides comprise both Rp and Sp blocks. In some embodiments, provided oligonucleotides comprise one or more Rp but no Sp blocks. In some embodiments, provided oligonucleotides comprise one or more Sp but no Rp blocks. In some embodiments, provided oligonucleotides comprise one or more PO blocks wherein each internucleotidic linkage in a natural phosphate linkage.

In some embodiments, compound of the disclosure comprises a 5′-block is an Sp block wherein each sugar moiety comprises a 2′-F modification. In some embodiments, a 5′-block is an Sp block wherein each of internucleotidic linkage is a modified internucleotidic linkage and each sugar moiety comprises a 2′-F modification. In some embodiments, a 5′-block is an Sp block wherein each of internucleotidic linkage is a phosphorothioate linkage and each sugar moiety comprises a 2′-F modification. In some embodiments, a 5′-block comprises 4 or more nucleoside units. In some embodiments, a 5′-block comprises 5 or more nucleoside units. In some embodiments, a 5′-block comprises 6 or more nucleoside units. In some embodiments, a 5′-block comprises 7 or more nucleoside units. In some embodiments, a 3′-block is an Sp block wherein each sugar moiety comprises a 2′-F modification. In some embodiments, a 3′-block is an Sp block wherein each of internucleotidic linkage is a modified internucleotidic linkage and each sugar moiety comprises a 2′-F modification. In some embodiments, a 3′-block is an Sp block wherein each of internucleotidic linkage is a phosphorothioate linkage and each sugar moiety comprises a 2′-F modification. In some embodiments, a 3′-block comprises 4 or more nucleoside units. In some embodiments, a 3′-block comprises 5 or more nucleoside units. In some embodiments, a 3′-block comprises 6 or more nucleoside units. In some embodiments, a 3′-block comprises 7 or more nucleoside units.

In some embodiments, compound of the disclosure comprises a type of nucleoside in a region or an oligonucleotide is followed by a specific type of internucleotidic linkage, e.g., natural phosphate linkage, modified internucleotidic linkage, Rp chiral internucleotidic linkage, Sp chiral internucleotidic linkage, etc. In some embodiments, A is followed by Sp. In some embodiments, A is followed by Rp. In some embodiments, A is followed by natural phosphate linkage (PO). In some embodiments, U is followed by Sp. In some embodiments, U is followed by Rp. In some embodiments, U is followed by natural phosphate linkage (PO). In some embodiments, C is followed by Sp. In some embodiments, C is followed by Rp. In some embodiments, C is followed by natural phosphate linkage (PO). In some embodiments, G is followed by Sp. In some embodiments, G is followed by Rp. In some embodiments, G is followed by natural phosphate linkage (PO). In some embodiments, C and U are followed by Sp. In some embodiments, C and U are followed by Rp. In some embodiments, C and U are followed by natural phosphate linkage (PO). In some embodiments, A and G are followed by Sp. In some embodiments, A and G are followed by Rp.

In some embodiments, the antisense strand comprises phosphorothioate internucleotide linkages between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the antisense comprises 3, 4 or 5 phosphorothioate internucleotide linkages; (iii) the sense strand is conjugated with a ligand; (iv) the sense strand comprises 2, 3, 4 or 5 2′-fluoro modifications; (v) the sense strand comprises 1, 2, 3, 4 or 5 phosphorothioate internucleotide linkages; (vi) the dsRNA comprises at least four 2′-fluoro modifications; (vii) the dsRNA comprises a duplex region of 12-40 nucleotide pairs in length; and (viii) the dsRNA has a blunt end at 5′-end of the antisense strand.

In some embodiments, the antisense strand comprises phosphorothioate internucleotide linkages between nucleotide positions 1 and 2, between nucleotide positions 2 and 3, between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the sense strand is conjugated with a ligand; (iii) the sense strand comprises 2, 3, 4 or 5 2′-fluoro modifications; (iv) the sense strand comprises 1, 2, 3, 4 or 5 phosphorothioate internucleotide linkages; (v) the dsRNA comprises at least four 2′-fluoro modifications; (vi) the dsRNA comprises a duplex region of 12-40 nucleotide pairs in length; (vii) the dsRNA comprises a duplex region of 12-40 nucleotide pairs in length; and (viii) the dsRNA has a blunt end at 5′-end of the antisense strand.

In some embodiments, the sense strand comprises phosphorothioate internucleotide linkages between nucleotide positions 1 and 2, and between nucleotide positions 2 and 3, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six, seven or all eight) of the following characteristics: (i) the antisense comprises 2, 3, 4, 5 or 6 2′-fluoro modifications; (ii) the antisense comprises 1, 2, 3, 4 or 5 phosphorothioate internucleotide linkages; (iii) the sense strand is conjugated with a ligand; (iv) the sense strand comprises 2, 3, 4 or 5 2′-fluoro modifications; (v) the sense strand comprises 3, 4 or 5 phosphorothioate internucleotide linkages; (vi) the dsRNA comprises at least four 2′-fluoro modifications; (vii) the dsRNA comprises a duplex region of 12-40 nucleotide pairs in length; and (viii) the dsRNA has a blunt end at 5′-end of the antisense strand.

In some embodiments, the sense strand comprises phosphorothioate internucleotide linkages between nucleotide positions 1 and 2, and between nucleotide positions 2 and 3, the antisense strand comprises phosphorothioate internucleotide linkages between nucleotide positions 1 and 2, between nucleotide positions 2 and 3, between nucleotide positions 21 and 22, and between nucleotide positions 22 and 23, wherein the antisense strand contains at least one thermally destabilizing modification of the duplex located in the seed region of the antisense strand (i.e., at position 2-9 of the 5′-end of the antisense strand), and wherein the dsRNA optionally further has at least one (e.g., one, two, three, four, five, six or all seven) of the following characteristics: (i) the antisense comprises 2, 3, 4, 5 or 6 2′-fluoro modifications; (ii) the sense strand is conjugated with a ligand; (iii) the sense strand comprises 2, 3, 4 or 5 2′-fluoro modifications; (iv) the sense strand comprises 3, 4 or 5 phosphorothioate internucleotide linkages; (v) the dsRNA comprises at least four 2′-fluoro modifications; (vi) the dsRNA comprises a duplex region of 12-40 nucleotide pairs in length; and (vii) the dsRNA has a blunt end at 5′-end of the antisense strand.

In some embodiments, the dsRNA molecule of the disclosure comprises mismatch(es) with the target, within the duplex, or combinations thereof. The mismatch can occur in the overhang region or the duplex region. The base pair can be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In some embodiments, the dsRNA molecule of the disclosure comprises at least one of the first 1, 2, 3, 4, or 5 base pairs within the duplex regions from the 5′- end of the antisense strand can be chosen independently from the group of: A:U, G:U, I:C, and mismatched pairs, e.g., non-canonical or other than canonical pairings or pairings which include a universal base, to promote the dissociation of the antisense strand at the 5′-end of the duplex.

In some embodiments, the nucleotide at the 1 position within the duplex region from the 5′-end in the antisense strand is selected from the group consisting of A, dA, dU, U, and dT. Alternatively, at least one of the first 1, 2 or 3 base pair within the duplex region from the 5′- end of the antisense strand is an AU base pair. For example, the first base pair within the duplex region from the 5′- end of the antisense strand is an AU base pair.

It was found that introducing 4′-modified or 5′-modified nucleotide to the 3′-end of a phosphodiester (PO), phosphorothioate (PS), or phosphorodithioate (PS2) linkage of a dinucleotide at any position of single stranded or double stranded oligonucleotide can exert steric effect to the internucleotide linkage and, hence, protecting or stabilizing it against nucleases.

In some embodiments, 5′-modified nucleoside is introduced at the 3′-end of a dinucleotide at any position of single stranded or double stranded siRNA. For instance, a 5′-alkylated nucleoside may be introduced at the 3′-end of a dinucleotide at any position of single stranded or double stranded siRNA. The alkyl group at the 5′ position of the ribose sugar can be racemic or chirally pure R or S isomer. An exemplary 5′-alkylated nucleoside is 5′-methyl nucleoside. The 5′-methyl can be either racemic or chirally pure R or S isomer.

In some embodiments, 4′-modified nucleoside is introduced at the 3′-end of a dinucleotide at any position of single stranded or double stranded siRNA. For instance, a 4′-alkylated nucleoside may be introduced at the 3′-end of a dinucleotide at any position of single stranded or double stranded siRNA. The alkyl group at the 4′ position of the ribose sugar can be racemic or chirally pure R or S isomer. An exemplary 4′-alkylated nucleoside is 4′-methyl nucleoside. The 4′-methyl can be either racemic or chirally pure R or S isomer. Alternatively, a 4′-O-alkylated nucleoside may be introduced at the 3′-end of a dinucleotide at any position of single stranded or double stranded siRNA. The 4′-O-alkyl of the ribose sugar can be racemic or chirally pure R or S isomer. An exemplary 4′-O-alkylated nucleoside is 4′-O-methyl nucleoside. The 4′-O-methyl can be either racemic or chirally pure R or S isomer.

In some embodiments, 5′-alkylated nucleoside is introduced at any position on the sense strand or antisense strand of a dsRNA, and such modification maintains or improves potency of the dsRNA. The 5′-alkyl can be either racemic or chirally pure R or S isomer. An exemplary 5′-alkylated nucleoside is 5′-methyl nucleoside. The 5′-methyl can be either racemic or chirally pure R or S isomer.

In some embodiments, 4′-alkylated nucleoside is introduced at any position on the sense strand or antisense strand of a dsRNA, and such modification maintains or improves potency of the dsRNA. The 4′-alkyl can be either racemic or chirally pure R or S isomer. An exemplary 4′-alkylated nucleoside is 4′-methyl nucleoside. The 4′-methyl can be either racemic or chirally pure R or S isomer.

In some embodiments, 4′-O-alkylated nucleoside is introduced at any position on the sense strand or antisense strand of a dsRNA, and such modification maintains or improves potency of the dsRNA. The 5′-alkyl can be either racemic or chirally pure R or S isomer. An exemplary 4′-O-alkylated nucleoside is 4′-O-methyl nucleoside. The 4′-O-methyl can be either racemic or chirally pure R or S isomer.

In some embodiments, the dsRNA molecule of the disclosure can comprise 2′-5′ linkages (with 2′-H, 2′-OH and 2′-OMe and with P═O or P═S). For example, the 2′-5′ linkages modifications can be used to promote nuclease resistance or to inhibit binding of the sense to the antisense strand, or can be used at the 5′ end of the sense strand to avoid sense strand activation by RISC.

In another embodiment, the dsRNA molecule of the disclosure can comprise L sugars (e.g., L ribose, L-arabinose with 2′-H, 2′-OH and 2′-OMe). For example, these L sugars modifications can be used to promote nuclease resistance or to inhibit binding of the sense to the antisense strand, or can be used at the 5′ end of the sense strand to avoid sense strand activation by RISC.

Various publications describe multimeric siRNA which can all be used with the dsRNA of the disclosure. Such publications include WO2007/091269, US 7858769, WO2010/141511, WO2007/117686, WO2009/014887, and WO2011/031520 which are hereby incorporated by their entirely.

As described in more detail below, the RNAi agent that contains conjugations of one or more carbohydrate moieties to an RNAi agent can optimize one or more properties of the RNAi agent. In many cases, the carbohydrate moiety will be attached to a modified subunit of the RNAi agent. For example, the ribose sugar of one or more ribonucleotide subunits of a dsRNA agent can be replaced with another moiety, e.g., a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The ligand may be attached to the polynucleotide via a carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide and polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

The RNAi agents may be conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and and decalin; preferably, the acyclic group is selected from serinol backbone or diethanolamine backbone.

In certain specific embodiments, the RNAi agent for use in the methods of the disclosure is an agent selected from the group of agents listed in Tables 2, 3, or 4. These agents may further comprise a ligand.

### IV. iRNAs Conjugated to Ligands

Another modification of the RNA of an iRNA of the invention involves chemically linking to the iRNA one or more ligands, moieties or conjugates that enhance the activity, cellular distribution or cellular uptake of the iRNA, e.g., into a cell. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4: 1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10: 1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In certain embodiments, a ligand alters the distribution, targeting or lifetime of an iRNA agent into which it is incorporated. In some embodiments, a ligand provides an enhanced affinity for a selected target, e.g., molecule, cell or cell type, compartment, e.g., a cellular or organ compartment, tissue, organ or region of the body, as, e.g., compared to a species absent such a ligand. Typical ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an α helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. In certain embodiments, the ligand is a multivalent galactose, e.g., an N-acetyl-galactosamine.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, O3-(oleoyl)cholenic acid, dimethoxytrityl, or phenoxazine)and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the iRNA agent into the cell, for example, by disrupting the cell’s cytoskeleton, e.g., by disrupting the cell’s microtubules, microfilaments, or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to an iRNA as described herein acts as a pharmacokinetic modulator (PK modulator). PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g., oligonucleotides of about 5 bases, 10 bases, 15 bases or 20 bases, comprising multiple of phosphorothioate linkages in the backbone are also amenable to the present invention as ligands (e.g. as PK modulating ligands). In addition, aptamers that bind serum components (e.g. serum proteins) are also suitable for use as PK modulating ligands in the embodiments described herein.

Ligand-conjugated iRNAs of the invention may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present invention may be conveniently and routinely made through the well-known technique of solid-phase synthesis. Equipment for such synthesis is sold by several vendors including, for example, Applied Biosystems® (Foster City, Calif.). Any other means for such synthesis known in the art may additionally or alternatively be employed. It is also known to use similar techniques to prepare other oligonucleotides, such as the phosphorothioates and alkylated derivatives.

In the ligand-conjugated oligonucleotides and ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

### A. Lipid Conjugates

In certain embodiments, the ligand or conjugate is a lipid or lipid-based molecule. Such a lipid or lipid-based molecule can typically bind a serum protein, such as human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, naproxen or aspirin can be used. A lipid or lipid-based ligand can (a) increase resistance to degradation of the conjugate, (b) increase targeting or transport into a target cell or cell membrane, or (c) can be used to adjust binding to a serum protein, e.g., HSA.

A lipid-based ligand can be used to modulate, e.g., control (e.g., inhibit) the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In certain embodiments, the lipid-based ligand binds HSA. For example, the ligand can bind HSA with a sufficient affinity such that distribution of the conjugate to a non-kidney tissue is enhanced. However, the affinity is typically not so strong that the HSA-ligand binding cannot be reversed.

In certain embodiments, the lipid-based ligand binds HSA weakly or not at all, such that distribution of the conjugate to the kidney is enhanced. Other moieties that target to kidney cells can also be used in place of or in addition to the lipid-based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells. Also included are HSA and low density lipoprotein (LDL).

### B. Cell Permeation Agents

In another aspect, the ligand is a cell-permeation agent, such as a helical cell-permeation agent. In certain embodiments, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is typically an α-helical agent and can have a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 9). An RFGF analogue (e.g., amino acid sequence AALLPVLLAAP (SEQ ID NO: 10)) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO: 11)) and the Drosophila Antennapedia protein (RQIKIWFQNRRMKWKK (SEQ ID NO: 12)) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Typically, the peptide or peptidomimetic tethered to a dsRNA agent via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods of the invention may be linear or cyclic, and may be modified, e.g., glycosylated or methylated, to facilitate targeting to a specific tissue(s). RGD-containing peptides and peptidiomimetics may include D-amino acids, as well as synthetic RGD mimics. In addition to RGD, one can use other moieties that target the integrin ligand. In certain embodiments, conjugates of this ligand target PECAM-1 or VEGF.

An RGD peptide moiety can be used to target a particular cell type, e.g., a tumor cell, such as an endothelial tumor cell or a breast cancer tumor cell (Zitzmann et al., Cancer Res., 62:5139-43, 2002). An RGD peptide can facilitate targeting of an dsRNA agent to tumors of a variety of other tissues, including the lung, kidney, spleen, or liver (Aoki et al., Cancer Gene Therapy 8:783-787, 2001). Typically, the RGD peptide will facilitate targeting of an iRNA agent to the kidney. The RGD peptide can be linear or cyclic, and can be modified, e.g., glycosylated or methylated to facilitate targeting to specific tissues. For example, a glycosylated RGD peptide can deliver an iRNA agent to a tumor cell expressing αᵥβ₃ (Haubner et al., Jour. Nucl. Med., 42:326-336, 2001).

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α -defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

### C. Carbohydrate Conjugates

In some embodiments of the compositions and methods of the invention, an iRNA further comprises a carbohydrate. The carbohydrate conjugated iRNA are advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and tri-saccharides include sugars having two or three monosaccharide units (e.g., C5, C6, C7, or C8).

In certain embodiments, a carbohydrate conjugate comprises a monosaccharide.

In certain embodiments, the monosaccharide is an N-acetylgalactosamine (GalNAc). GalNAc conjugates, which comprise one or more N-acetylgalactosamine (GalNAc) derivatives, are described, for example, in US 8,106,022, the entire content of which is hereby incorporated herein by reference. In some embodiments, the GalNAc conjugate serves as a ligand that targets the iRNA to particular cells. In some embodiments, the GalNAc conjugate targets the iRNA to liver cells, e.g., by serving as a ligand for the asialoglycoprotein receptor of liver cells (e.g., hepatocytes).

In some embodiments, the carbohydrate conjugate comprises one or more GalNAc derivatives. The GalNAc derivatives may be attached via a linker, e.g., a bivalent or trivalent branched linker. In some embodiments the GalNAc conjugate is conjugated to the 3′ end of the sense strand. In some embodiments, the GalNAc conjugate is conjugated to the iRNA agent (e.g., to the 3′ end of the sense strand) via a linker, e.g., a linker as described herein. In some embodiments the GalNAc conjugate is conjugated to the 5′ end of the sense strand. In some embodiments, the GalNAc conjugate is conjugated to the iRNA agent (e.g., to the 5′ end of the sense strand) via a linker, e.g., a linker as described herein.

In certain embodiments of the invention, the GalNAc or GalNAc derivative is attached to an iRNA agent of the invention via a monovalent linker. In some embodiments, the GalNAc or GalNAc derivative is attached to an iRNA agent of the invention via a bivalent linker. In yet other embodiments of the invention, the GalNAc or GalNAc derivative is attached to an iRNA agent of the invention via a trivalent linker. In other embodiments of the invention, the GalNAc or GalNAc derivative is attached to an iRNA agent of the invention via a tetravalent linker.

In certain embodiments, the double stranded RNAi agents of the invention comprise one GalNAc or GalNAc derivative attached to the iRNA agent. In certain embodiments, the double stranded RNAi agents of the invention comprise a plurality (e.g., 2, 3, 4, 5, or 6) GalNAc or GalNAc derivatives, each independently attached to a plurality of nucleotides of the double stranded RNAi agent through a plurality of monovalent linkers.

In some embodiments, for example, when the two strands of an iRNA agent of the invention are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker. The hairpin loop may also be formed by an extended overhang in one strand of the duplex.

In some embodiments, for example, when the two strands of an iRNA agent of the invention are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker. The hairpin loop may also be formed by an extended overhang in one strand of the duplex.

In some embodiments, the GalNAc conjugate is

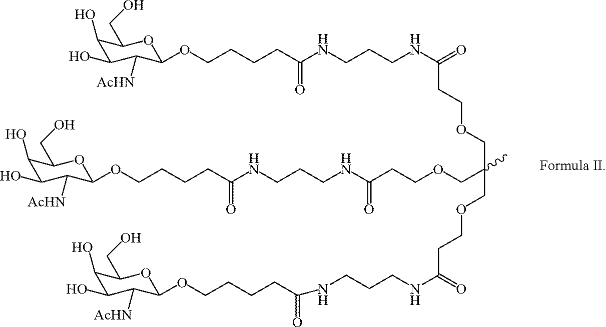 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the RNAi agent is attached to the carbohydrate conjugate via a linker as shown in the following schematic wherein X is O or S

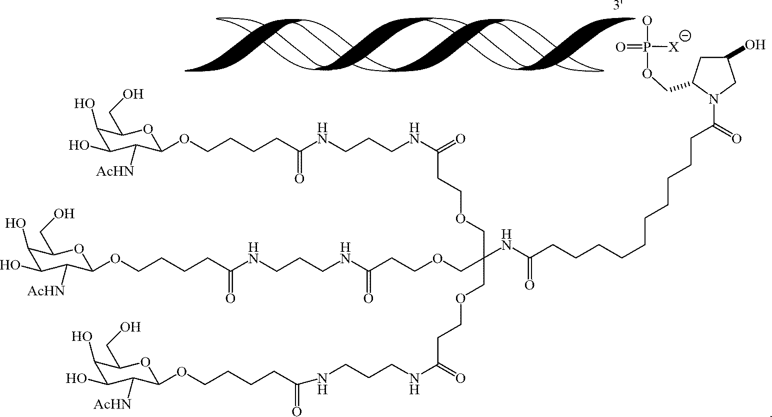

In some embodiments, the RNAi agent is conjugated to L96 as defined in Table 1 and shown below:

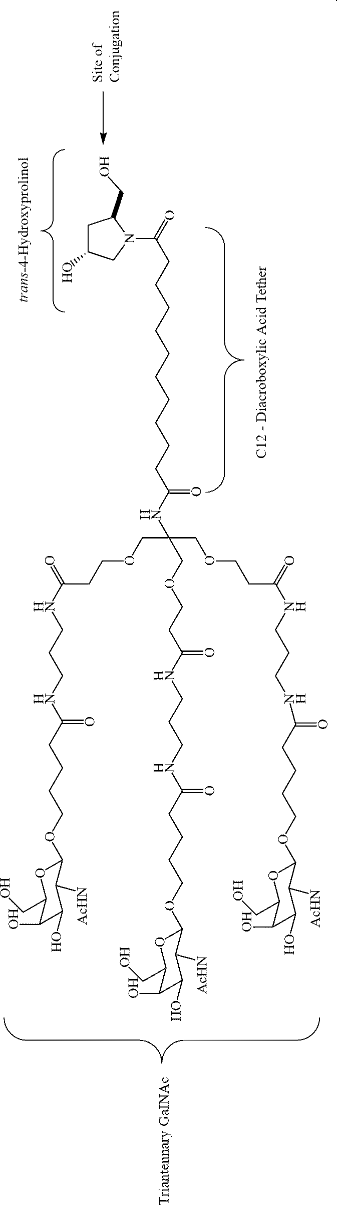

In certain embodiments, a carbohydrate conjugate for use in the compositions and methods of the invention is selected from the group consisting of:

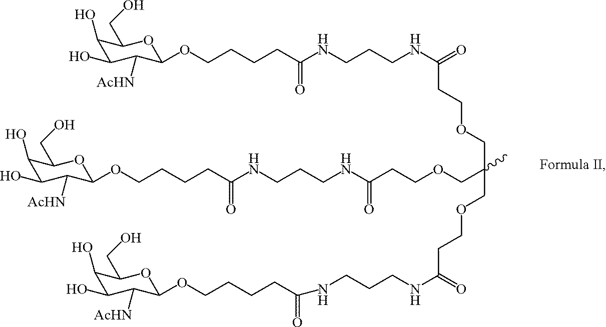

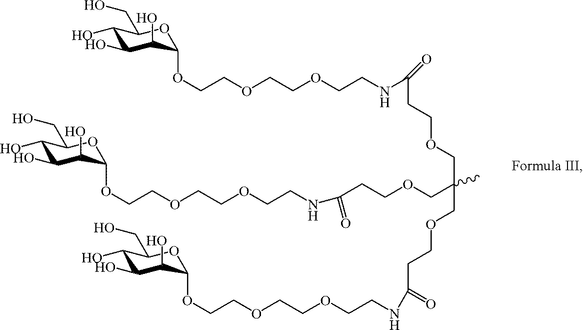

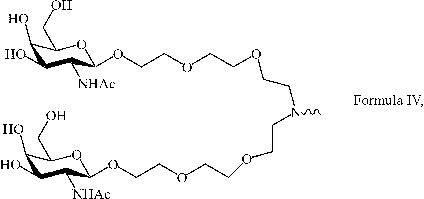

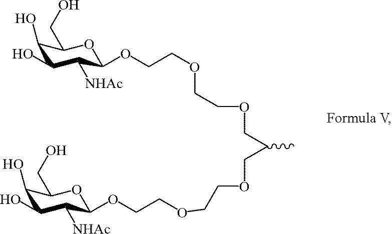

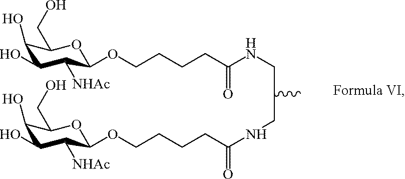

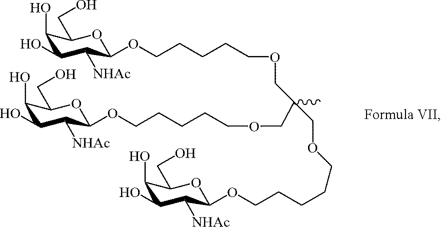

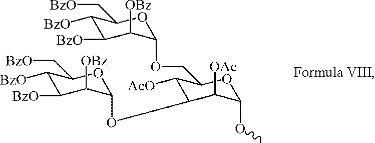

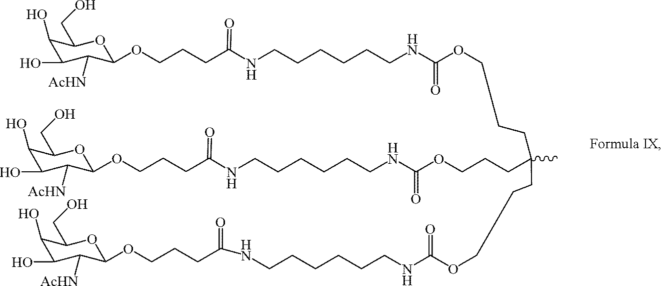

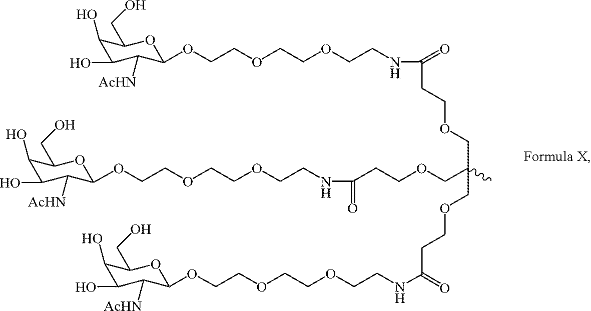

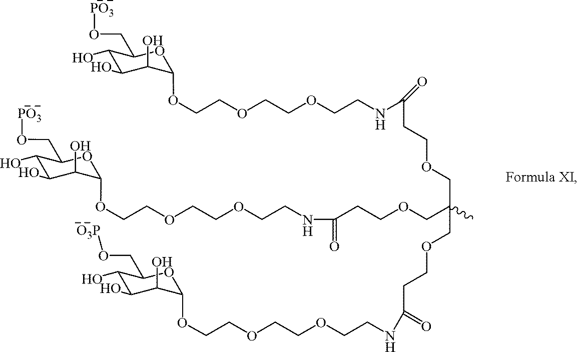

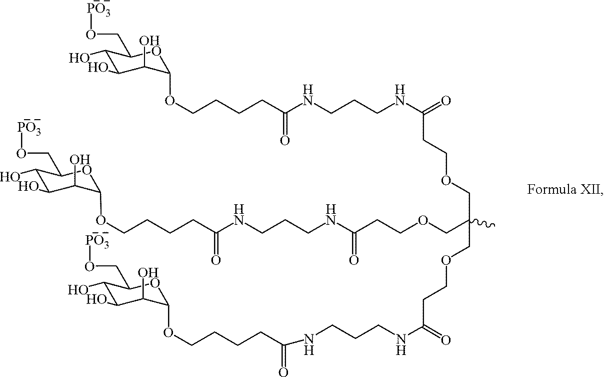

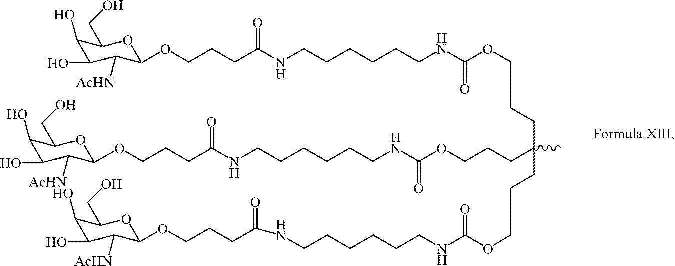

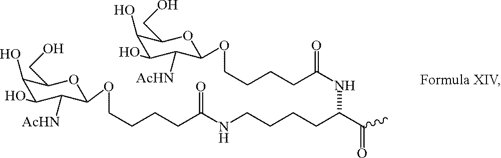

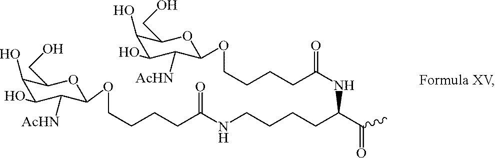

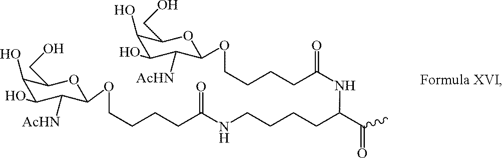

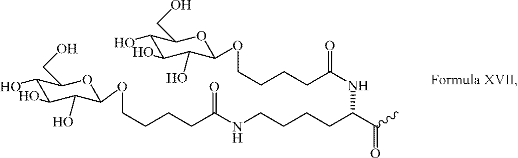

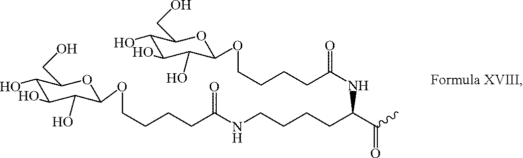

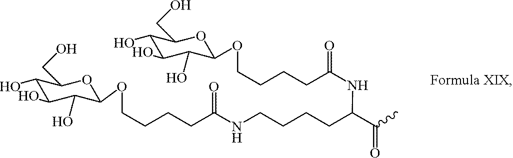

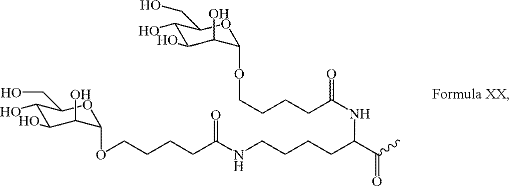

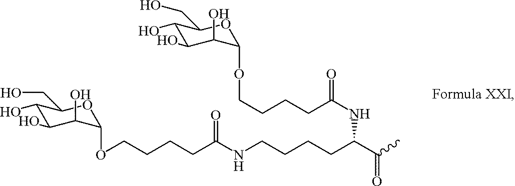

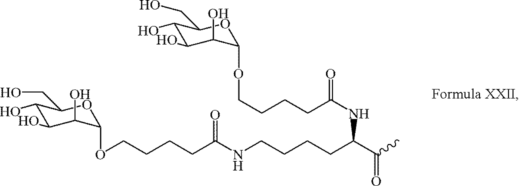

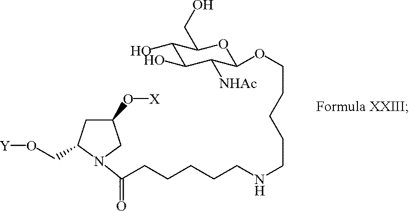

wherein Y is O or S and n is 3 -6

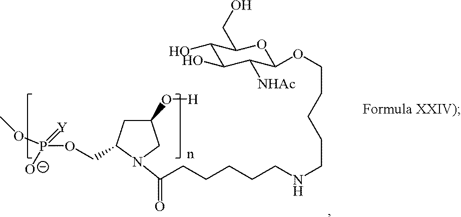

wherein Y is O or S and n is 3-6

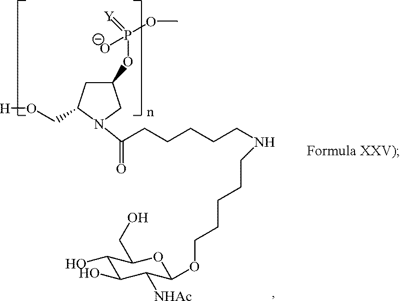

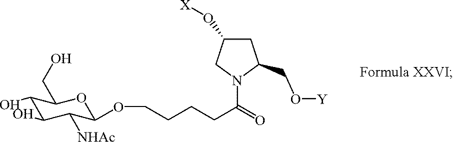

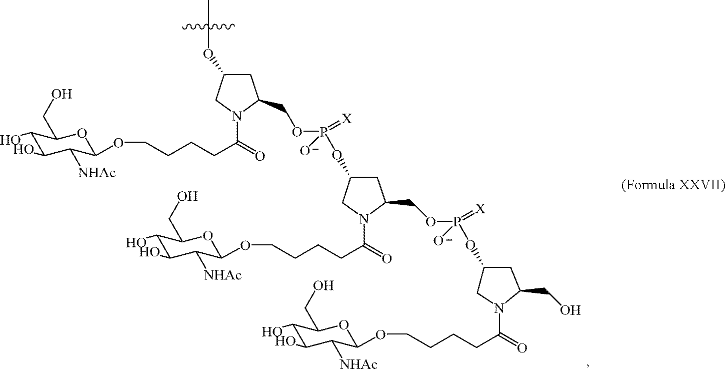

wherein X is O or S

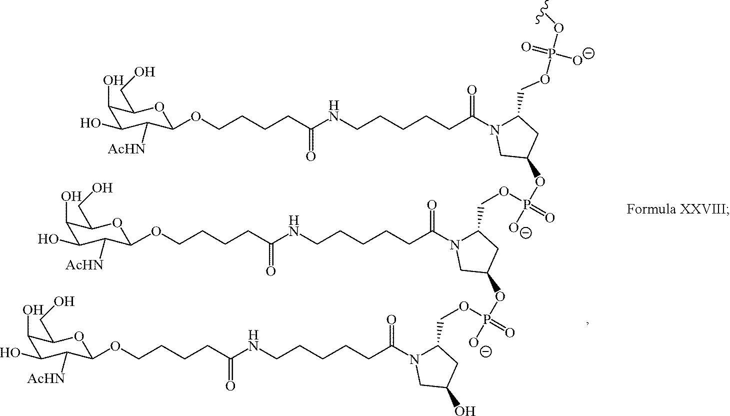

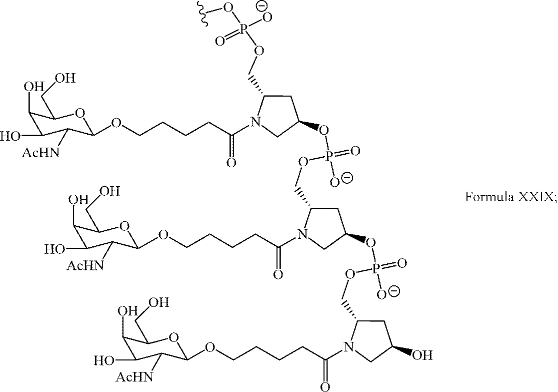

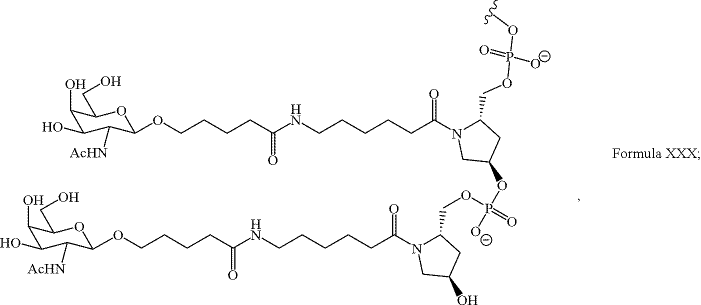

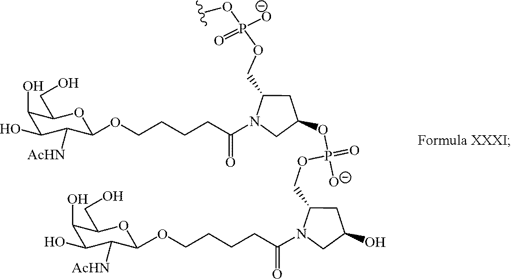

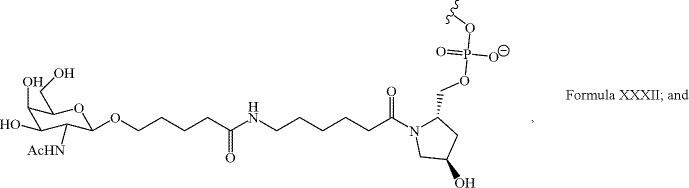

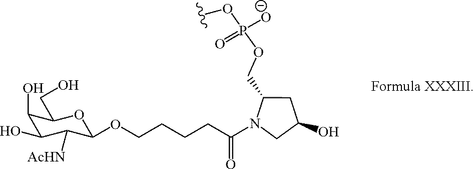

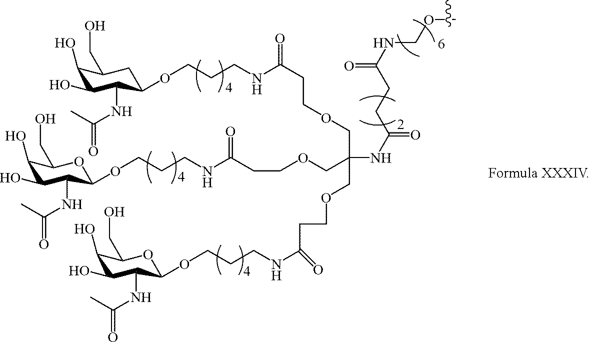

In certain embodiments, a carbohydrate conjugate for use in the compositions and methods of the invention is a monosaccharide. In certain embodiments, the monosaccharide is an N-acetylgalactosamine, such as

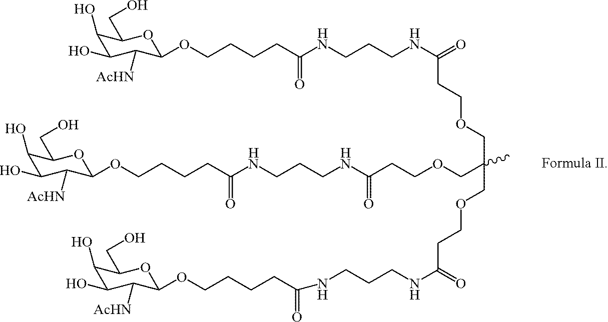 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

Another representative carbohydrate conjugate for use in the embodiments described herein includes, but is not limited to,

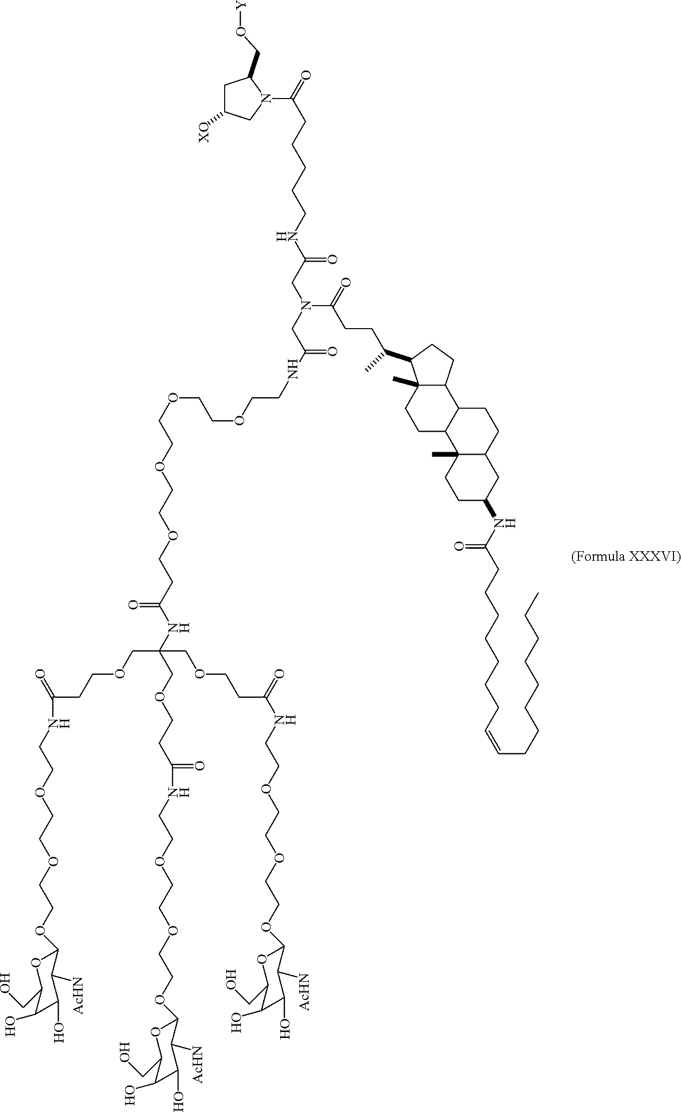

when one of X or Y is an oligonucleotide, the other is a hydrogen.

In some embodiments, a suitable ligand is a ligand disclosed in WO 2019/055633, the entire contents of which are incorporated herein by reference. In one embodiment the ligand comprises the structure below:

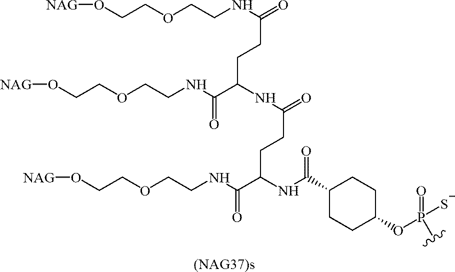

In certain embodiments, the RNAi agents of the disclosure may include GalNAc ligands, even if such GalNAc ligands are currently projected to be of limited value for intrathecal/CNS delivery route(s) of the instant disclosure.

In certain embodiments of the invention, the GalNAc or GalNAc derivative is attached to an iRNA agent of the invention via a monovalent linker. In some embodiments, the GalNAc or GalNAc derivative is attached to an iRNA agent of the invention via a bivalent linker. In yet other embodiments of the invention, the GalNAc or GalNAc derivative is attached to an iRNA agent of the invention via a trivalent linker.

In one embodiment, the double stranded RNAi agents of the invention comprise one or more GalNAc or GalNAc derivative attached to the iRNA agent. The GalNAc may be attached to any nucleotide via a linker on the sense strand or antisense strand. The GalNac may be attached to the 5′-end of the sense strand, the 3′ end of the sense strand, the 5′-end of the antisense strand, or the 3′ -end of the antisense strand. In one embodiment, the GalNAc is attached to the 3′ end of the sense strand, e.g., via a trivalent linker.

In other embodiments, the double stranded RNAi agents of the invention comprise a plurality (e.g., 2, 3, 4, 5, or 6) GalNAc or GalNAc derivatives, each independently attached to a plurality of nucleotides of the double stranded RNAi agent through a plurality of linkers, e.g., monovalent linkers.

In some embodiments, for example, when the two strands of an iRNA agent of the invention is part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker.

In some embodiments, the carbohydrate conjugate further comprises one or more additional ligands as described above, such as, but not limited to, a PK modulator or a cell permeation peptide.

Additional carbohydrate conjugates and linkers suitable for use in the present invention include those described in WO 2014/179620 and WO 2014/179627, the entire contents of each of which are incorporated herein by reference.

### D. Linkers

In some embodiments, the conjugate or ligand described herein can be attached to an iRNA oligonucleotide with various linkers that can be cleavable or non-cleavable.

The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, substituted or unsubstituted heterocyclic; where R8 is hydrogen, acyl, aliphatic or substituted aliphatic. In certain embodiments, the linker is between about 1-24 atoms, 2-24, 3-24, 4-24, 5-24, 6-24, 6-18, 7-18, 8-18 atoms, 7-17, 8-17, 6-16, 7-16, or 8-16 atoms.

A cleavable linking group is one which is sufficiently stable outside the cell, but which upon entry into a target cell is cleaved to release the two parts the linker is holding together. In another embodiment, the cleavable linking group is cleaved at least about 10 times, 20, times, 30 times, 40 times, 50 times, 60 times, 70 times, 80 times, 90 times or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a selected pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In certain embodiments, useful candidate compounds are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

### I. Redox Cleavable Linking Groups

In certain embodiments, a cleavable linking group is a redox cleavable linking group that is cleaved upon reduction or oxidation. An example of reductively cleavable linking group is a disulphide linking group (-S-S-). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

### Ii. Phosphate-Based Cleavable Linking Groups

In certain embodiments, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are -O-P(O)(ORk)-O-, -OP(S)(ORk)-O-, -O-P(S)(SRk)-O-, -S-P(O)(ORk)-O-, -O-P(O)(ORk)-S-, -S-P(O)(ORk)-S-, -OP(S)(ORk)-S-, -S-P(S)(ORk)-O-, -O-P(O)(Rk)-O-, -O-P(S)(Rk)-O-, -S-P(O)(Rk)-O-, -S-P(S)(Rk)-O-, -S-P(O)(Rk)-S-, -O-P(S)(Rk)-S-, wherein Rk at each occurrence can be, independently, C1-C20 alkyl, C1-C20 haloalkyl, C6-C10 aryl, or C7-C12 aralkyl. Additional embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O, —S—P(S)(H)—O—, —S—P(O)(H)—S—, —O—P(S)(H)—S—. In certain embodiments, a phosphate-based linking group is —O—P(O)(OH)—O—. These candidates can be evaluated using methods analogous to those described above.

### Iii. Acid Cleavable Linking Groups

In certain embodiments, a cleavable linker comprises an acid cleavable linking group. An acid cleavable linking group is a linking group that is cleaved under acidic conditions. In one embodiment, acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). Another embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

### Iv. Ester-Based Cleavable Linking Groups

In certain embodiments, a cleavable linker comprises an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

### V. Peptide-Based Cleavable Linking Groups

In yet another embodiment, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula - NHCHRAC(O)NHCHRBC(O)-, where RA and RB are the R groups of the two adjacent amino acids. These candidates can be evaluated using methods analogous to those described above.

In some embodiments, an iRNA of the invention is conjugated to a carbohydrate through a linker. Non-limiting examples of iRNA carbohydrate conjugates with linkers of the compositions and methods of the invention include, but are not limited to,

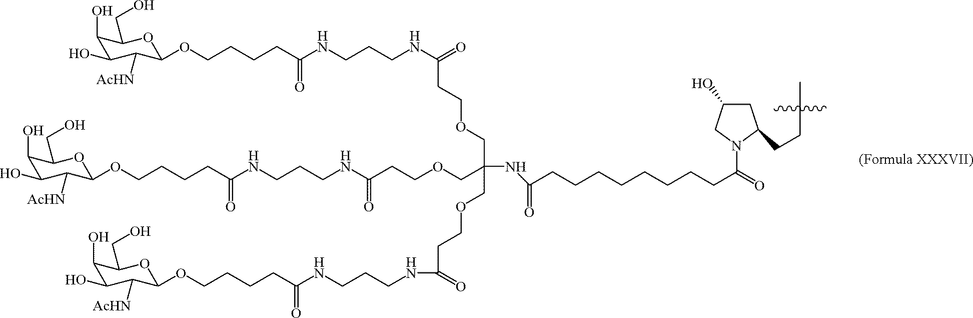 [SMILES: *CC[C@@H]1C[C@@H](O)CN1C(=O)CCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

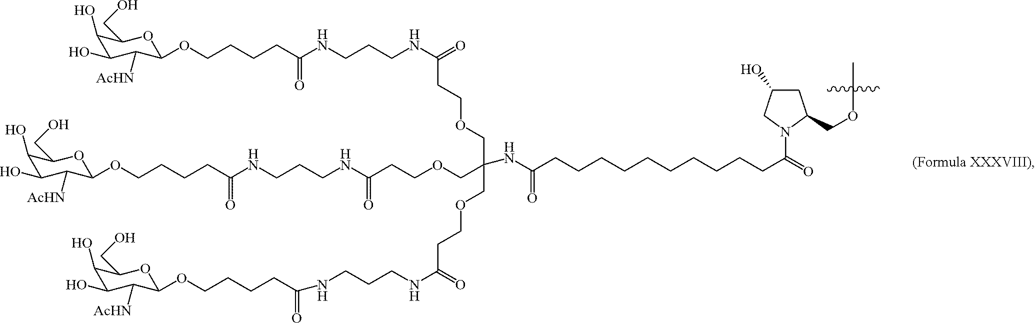 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

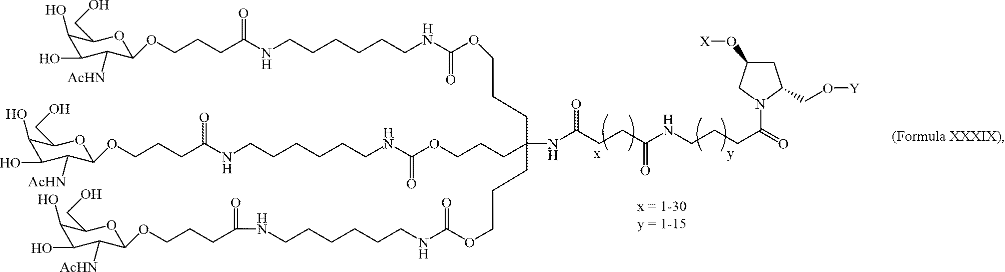

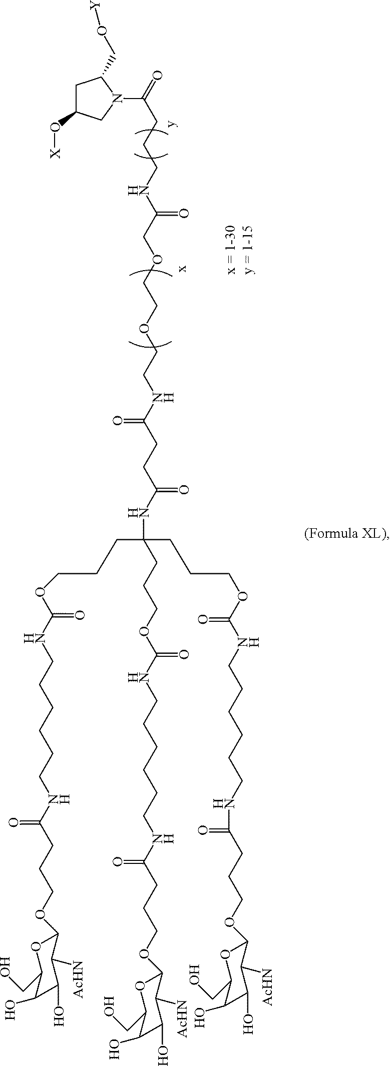

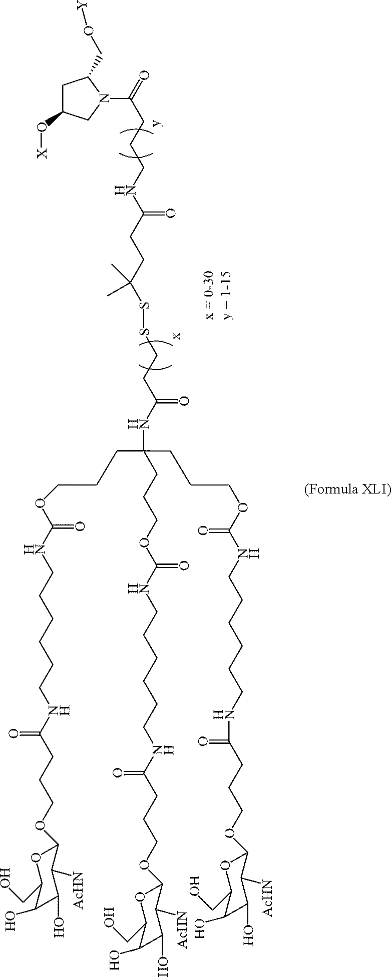

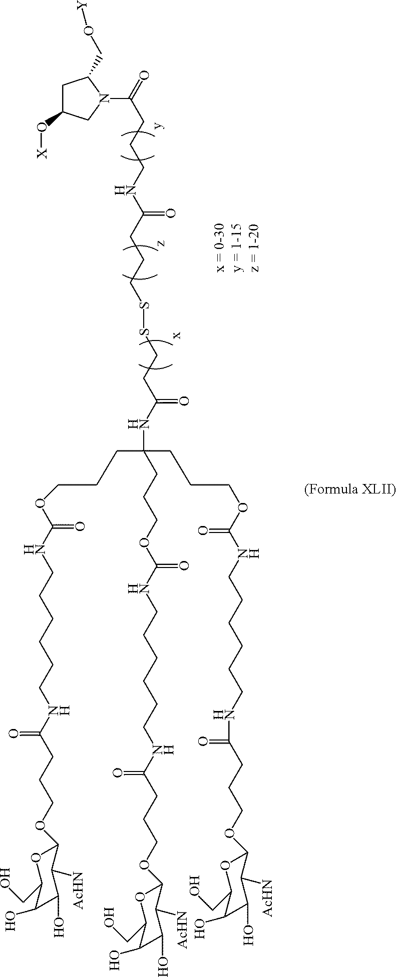

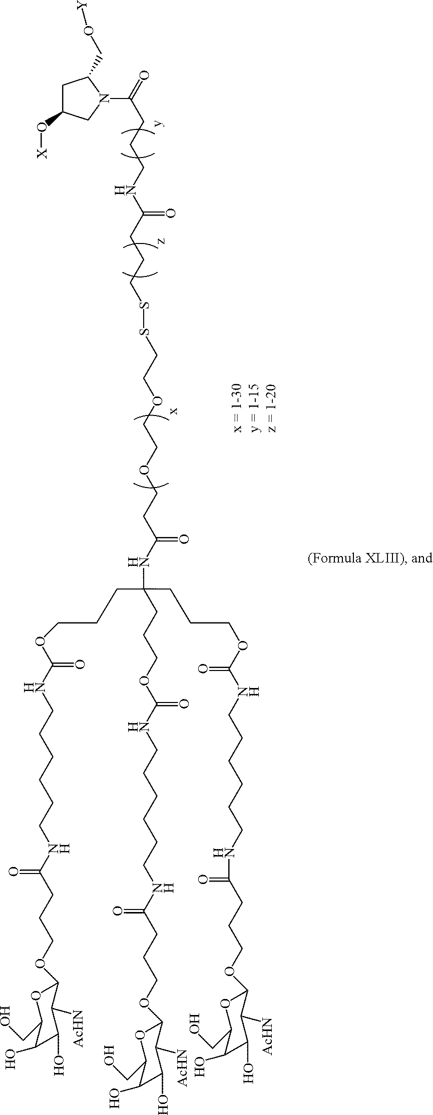

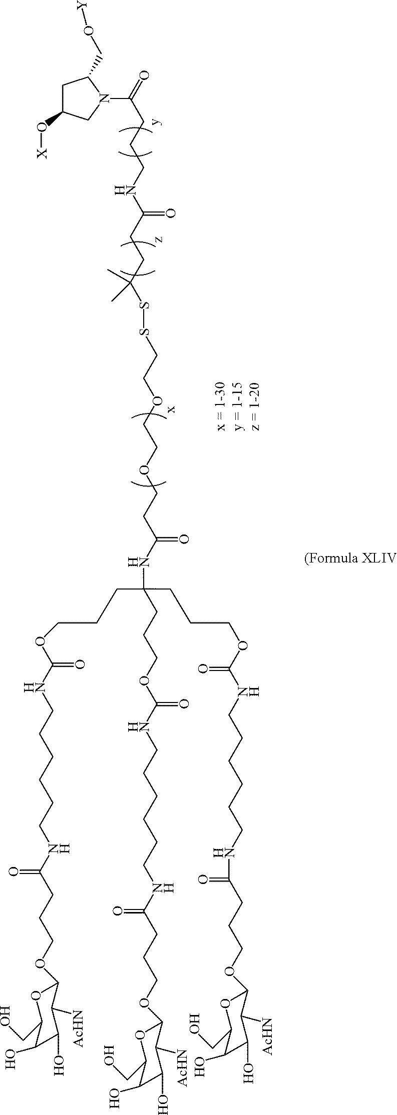

, when one of X or Y is an oligonucleotide, the other is a hydrogen.

In certain embodiments of the compositions and methods of the invention, a ligand is one or more “GalNAc” (N-acetylgalactosamine) derivatives attached through a bivalent or trivalent branched linker.

In certain embodiments, a dsRNA of the invention is conjugated to a bivalent or trivalent branched linker selected from the group of structures shown in any of formula (XLV) - (XLVI):

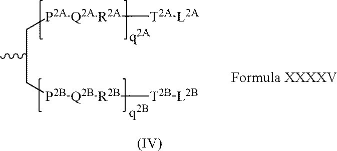

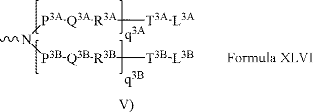

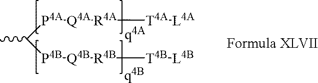

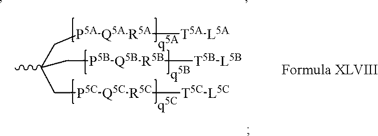

wherein:
q2A, q2B, q3A, q3B, q4A, q4B, q5A, q5B and q5C represent independently for each occurrence 0-20 and wherein the repeating unit can be the same or different;
P^{2A,} P^{2B,} P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{4A}, T^{5B}, T^{5C} are each independently for each occurrence absent, CO, NH, O, S, OC(O), NHC(O), CH₂, CH₂NH or CH₂O;
Q^{2A,} Q^{2B,} Q^{3A}, Q^{3B}, Q^{4A}, Q^{4B}, Q^{5A}, Q^{5B}, Q^{5C} are independently for each occurrence absent, alkylene, substituted alkylene wherein one or more methylenes can be interrupted or terminated by one or more of O, S, S(O), SO₂, N(R^{N}), C(R′)=C(R′’), C═C or C(O);
R^{2A}, R^{2B}, R^{3A}, R^{3B}, R^{4A}, R^{4B}, R^{5A}, R^{5B}, R^{5C} are each independently for each occurrence absent, NH, O, S, CH₂, C(O)O, C(O)NH, NHCH(R^{a})C(O), -C(O)-CH(R^{a})-NH-, CO, CH═N—O,

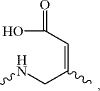

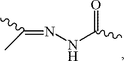

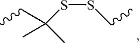

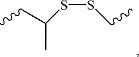

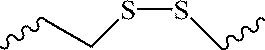

or heterocyclyl;
L^{2A}, L^{2B}, L^{3A}, L^{3B}, L^{4A}, L^{4B}, L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and R^{a} is H or amino acid side chain. Trivalent conjugating GalNAc derivatives are particularly useful for use with RNAi agents for inhibiting the expression of a target gene, such as those of formula (XLIX):

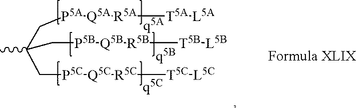

wherein L^{5A}, L^{5B} and L^{5C} represent a monosaccharide, such as GalNAc derivative.

Examples of suitable bivalent and trivalent branched linker groups conjugating GalNAc derivatives include, but are not limited to, the structures recited above as formulas II, VII, XI, X, and XIII.

Representative U.S. Patents that teach the preparation of RNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,688,941; 6,294,664; 6,320,017; 6,576,752; 6,783,931; 6,900,297; 7,037,646; and 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within an iRNA. The present invention also includes iRNA compounds that are chimeric compounds.

“Chimeric” iRNA compounds or “chimeras,” in the context of this invention, are iRNA compounds, preferably dsRNA agents, that contain two or more chemically distinct regions, each made up of at least one monomer unit, i.e., a nucleotide in the case of a dsRNA compound. These iRNAs typically contain at least one region wherein the RNA is modified so as to confer upon the iRNA increased resistance to nuclease degradation, increased cellular uptake, or increased binding affinity for the target nucleic acid. An additional region of the iRNA can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of iRNA inhibition of gene expression. Consequently, comparable results can often be obtained with shorter iRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the RNA of an iRNA can be modified by a non-ligand group. A number of non-ligand molecules have been conjugated to iRNAs in order to enhance the activity, cellular distribution or cellular uptake of the iRNA, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4: 1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:365 1; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such RNA conjugates have been listed above. Typical conjugation protocols involve the synthesis of RNAs bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

### V. Delivery of an RNAi Agent of the Disclosure

The delivery of an RNAi agent of the disclosure to a cell e.g., a cell within a subject, such as a human subject (e.g., a subject in need thereof, such as a subject having an ALK-associated disorder, e.g., type 2 diabetes, obesity, or an obesity-associated disease, can be achieved in a number of different ways. For example, delivery may be performed by contacting a cell with an RNAi agent of the disclosure either in vitro or in vivo. In vivo delivery may also be performed directly by administering a composition comprising an RNAi agent, e.g., a dsRNA, to a subject. Alternatively, in vivo delivery may be performed indirectly by administering one or more vectors that encode and direct the expression of the RNAi agent. These alternatives are discussed further below.

In general, any method of delivering a nucleic acid molecule (in vitro or in vivo) can be adapted for use with an RNAi agent of the disclosure (see e.g., Akhtar S. and Julian RL., (1992) Trends Cell. Biol. 2(5): 139-144 and WO94/02595, which are incorporated herein by reference in their entireties). For in vivo delivery, factors to consider in order to deliver an RNAi agent include, for example, biological stability of the delivered agent, prevention of non-specific effects, and accumulation of the delivered agent in the target tissue. The non-specific effects of an RNAi agent can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the RNAi agent to be administered. Several studies have shown successful knockdown of gene products when an RNAi agent is administered locally. For example, intraocular delivery of a VEGF dsRNA by intravitreal injection in cynomolgus monkeys (Tolentino, MJ. et al., (2004) Retina 24:132-138) and subretinal injections in mice (Reich, SJ. et al. (2003) Mol. Vis. 9:210-216) were both shown to prevent neovascularization in an experimental model of age-related macular degeneration. In addition, direct intratumoral injection of a dsRNA in mice reduces tumor volume (Pille, J. et al. (2005) Mol. Ther. 11:267-274) and can prolong survival of tumor-bearing mice (Kim, WJ. et al., (2006) Mol. Ther. 14:343-350; Li, S. et al., (2007) Mol. Ther. 15:515-523). RNA interference has also shown success with local delivery to the CNS by direct injection (Dorn, G. et al., (2004) Nucleic Acids 32:e49; Tan, PH. et al. (2005) Gene Ther. 12:59-66; Makimura, H. et al. (2002) BMC Neurosci. 3:18; Shishkina, GT., et al. (2004) Neuroscience 129:521-528; Thakker, ER., et al. (2004) Proc. Natl. Acad. Sci. U.S.A. 101:17270-17275; Akaneya,Y., et al. (2005) J. Neurophysiol. 93:594-602) and to the lungs by intranasal administration (Howard, KA. et al., (2006) Mol. Ther. 14:476-484; Zhang, X. et al., (2004) J. Biol. Chem. 279: 10677-10684; Bitko, V. et al., (2005) Nat. Med. 11:50-55). For administering an RNAi agent systemically for the treatment of a disease, the RNA can be modified or alternatively delivered using a drug delivery system; both methods act to prevent the rapid degradation of the dsRNA by endo- and exo-nucleases in vivo. Modification of the RNA or the pharmaceutical carrier can also permit targeting of the RNAi agent to the target tissue and avoid undesirable off-target effects (e.g., without wishing to be bound by theory, use of GNAs as described herein has been identified to destabilize the seed region of a dsRNA, resulting in enhanced preference of such dsRNAs for on-target effectiveness, relative to off-target effects, as such off-target effects are significantly weakened by such seed region destabilization). RNAi agents can be modified by chemical conjugation to lipophilic groups such as cholesterol to enhance cellular uptake and prevent degradation. For example, an RNAi agent directed against ApoB conjugated to a lipophilic cholesterol moiety was injected systemically into mice and resulted in knockdown of apoB mRNA in both the liver and jejunum (Soutschek, J. et al., (2004) Nature 432:173-178). Conjugation of an RNAi agent to an aptamer has been shown to inhibit tumor growth and mediate tumor regression in a mouse model of prostate cancer (McNamara, JO. et al., (2006) Nat. Biotechnol. 24: 1005-1015). In an alternative embodiment, the RNAi agent can be delivered using drug delivery systems such as a nanoparticle, a dendrimer, a polymer, liposomes, or a cationic delivery system. Positively charged cationic delivery systems facilitate binding of molecule RNAi agent (negatively charged) and also enhance interactions at the negatively charged cell membrane to permit efficient uptake of an RNAi agent by the cell. Cationic lipids, dendrimers, or polymers can either be bound to an RNAi agent, or induced to form a vesicle or micelle (see e.g., Kim SH. et al., (2008) Journal of Controlled Release 129(2): 107-116) that encases an RNAi agent. The formation of vesicles or micelles further prevents degradation of the RNAi agent when administered systemically. Methods for making and administering cationic- RNAi agent complexes are well within the abilities of one skilled in the art (see e.g., Sorensen, DR., et al. (2003) J. Mol. Biol 327:761-766; Verma, UN. et al., (2003) Clin. Cancer Res. 9: 1291-1300; Arnold, AS et al. (2007) J. Hypertens. 25: 197-205, which are incorporated herein by reference in their entirety). Some non-limiting examples of drug delivery systems useful for systemic delivery of RNAi agents include DOTAP (Sorensen, DR., et al (2003), supra; Verma, UN. et al., (2003), supra), Oligofectamine, “solid nucleic acid lipid particles” (Zimmermann, TS. et al., (2006) Nature 441: 111-114), cardiolipin (Chien, PY. et al., (2005) Cancer Gene Ther. 12:321-328; Pal, A. et al., (2005) Int J. Oncol. 26: 1087-1091), polyethyleneimine (Bonnet ME. et al., (2008) Pharm. Res. Aug 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, DA. et al., (2007) Biochem. Soc. Trans. 35:61-67; Yoo, H. et al., (1999) Pharm. Res. 16: 1799-1804). In some embodiments, an RNAi agent forms a complex with cyclodextrin for systemic administration. Methods for administration and pharmaceutical compositions of RNAi agents and cyclodextrins can be found in U.S. Pat. No. 7,427,605, which is herein incorporated by reference in its entirety.

Certain aspects of the instant disclosure relate to a method of reducing the expression of an ALK target gene in a cell, comprising contacting said cell with the double-stranded RNAi agent of the disclosure. In one embodiment, the cell is a CNS cell, such as a brain cell. In other embodiment, the cell is an adipocyte cell.

Another aspect of the disclosure relates to a method of reducing the expression of an ALK target gene in a subject, comprising administering to the subject the double-stranded RNAi agent of the disclosure.

Another aspect of the disclosure relates to a method of treating a subject having type 2 diabetes, obesity, or an obesity-associated disorder, comprising administering to the subject a therapeutically effective amount of the double-stranded ALK-targeting RNAi agent of the disclosure, thereby treating the subject. Exemplary obesity-associated disorders that can be treated by the method of the disclosure include type 2 diabetes, hypertension, dyslipidemia, atherosclerosis, coronary heart disease, stroke, metabolic syndrome, gallbladder disease, fatty liver, osteoarthritis, sleep apnea, breathing problems, various types of cancer (e.g., endometrial cancer, esophageal adenocarcinoma, gastric cardia cancer, liver cancer, kidney cancer, pancreatic cancer), mental illness (e.g., depression, anxiety), body pain, and difficulty with physical functioning.

Another aspect of the disclosure relates to a method of preventing a sign or a symptom in a subject having type 2 diabetes, obesity, or an obesity-associated disorder, comprising administering to the subject a therapeutically effective amount of the double-stranded ALK-targeting RNAi agent of the disclosure, thereby preventing a sign or symptom in the subject that would benefit from reduction in ALK expression. Exemplary obesity-associated sign or symptom include difficulty moving around, difficulty breathing, chest tightness, headache, sleepiness, feeling tired, and feeling depressed.

Another aspect of the disclosure relates to a method of preventing a subject from having type 2 diabetes, obesity, or an obesity-associated disorder, comprising administering to the subject a therapeutically effective amount of the double-stranded ALK-targeting RNAi agent of the disclosure, thereby preventing the subject from having type 2 diabetes, obesity, or an obesity-associated disorder.

Another aspect of the disclosure relates to a method of supporting weight loss or weight maintenance in a subject, comprising administering to the subject a therapeutically effective amount of the double-stranded ALK-targeting RNAi agent of the disclosure. In one embodiment, the invention provides a non-therapeutic use of an agent of the invention to maintain a healthy body composition, for example after a period of weight loss.

In one embodiment, the double-stranded RNAi agent is administered intrathecally. By intrathecal administration of the double-stranded RNAi agent, the method can reduce the expression of an ALK target gene in a brain (e.g., hypothalamus and pituitary gland) or spine tissue, (e.g., cervical spine, lumbar spine, and thoracic spine).

For ease of exposition the formulations, compositions and methods in this section are discussed largely with regard to modified siRNA compounds. It may be understood, however, that these formulations, compositions and methods can be practiced with other siRNA compounds, e.g., unmodified siRNA compounds, and such practice is within the disclosure. A composition that includes an RNAi agent can be delivered to a subject by a variety of routes. Exemplary routes include: intrathecal, intravenous, topical, rectal, anal, vaginal, nasal, pulmonary, and ocular.

The RNAi agents of the disclosure can be incorporated into pharmaceutical compositions suitable for administration. Such compositions typically include one or more species of RNAi agent and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present disclosure may be administered in a number of ways depending upon whether local or systemic treatment is desired and upon the area to be treated. Administration may be topical (including ophthalmic, vaginal, rectal, intranasal, transdermal), intrathecal, oral, or parenteral. Parenteral administration includes intravenous drip, subcutaneous, intraperitoneal or intramuscular injection, or intrathecal or intraventricular administration.

The route and site of administration may be chosen to enhance targeting. For example, to target neural or spinal tissue, intrathecal injection would be a logical choice. Lung cells might be targeted by administering the RNAi agent in aerosol form. The vascular endothelial cells could be targeted by coating a balloon catheter with the RNAi agent and mechanically introducing the RNA.

Formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable. Coated condoms, gloves and the like may also be useful.

Compositions for oral administration include powders or granules, suspensions or solutions in water, syrups, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening or flavoring agents can be added.

Compositions for intrathecal or intraventricular administration may include sterile aqueous solutions which may also contain buffers, diluents, and other suitable additives.

Formulations for parenteral administration may include sterile aqueous solutions which may also contain buffers, diluents, and other suitable additives. Intraventricular injection may be facilitated by an intraventricular catheter, for example, attached to a reservoir. For intravenous use, the total concentration of solutes may be controlled to render the preparation isotonic.

In one embodiment, the administration of the siRNA compound, e.g., a double-stranded siRNA compound, or ssiRNA compound, composition is parenteral, e.g., intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral, or ocular. Administration can be provided by the subject or by another person, e.g., a health care provider. The medication can be provided in measured doses or in a dispenser which delivers a metered dose. Selected modes of delivery are discussed in more detail below.

### A. Intrathecal Administration

In one embodiment, the double-stranded RNAi agent is delivered by intrathecal injection (i.e., injection into the spinal fluid which bathes the brain and spinal cord tissue). Intrathecal injection of RNAi agents into the spinal fluid can be performed as a bolus injection or via minipumps which can be implanted beneath the skin, providing a regular and constant delivery of siRNA into the spinal fluid. The circulation of the spinal fluid from the choroid plexus, where it is produced, down around the spinal cord and dorsal root ganglia and subsequently up past the cerebellum and over the cortex to the arachnoid granulations, where the fluid can exit the CNS, that, depending upon size, stability, and solubility of the compounds injected, molecules delivered intrathecally could hit targets throughout the entire CNS.

In some embodiments, the intrathecal administration is via a pump. The pump may be a surgically implanted osmotic pump. In one embodiment, the osmotic pump is implanted into the subarachnoid space of the spinal canal to facilitate intrathecal administration.

In some embodiments, the intrathecal administration is via an intrathecal delivery system for a pharmaceutical including a reservoir containing a volume of the pharmaceutical agent, and a pump configured to deliver a portion of the pharmaceutical agent contained in the reservoir. More details about this intrathecal delivery system may be found in WO 2015/116658, which is incorporated by reference in its entirety.

The amount of intrathecally injected RNAi agents may vary from one target gene to another target gene and the appropriate amount that has to be applied may have to be determined individually for each target gene. Typically, this amount ranges from 10 µg to 2 mg, preferably 50 µg to 1500 µg, more preferably 100 µg to 1000 µg.

### B. Vector Encoded RNAi Agents of the Disclosure

RNAi agents targeting the ALK gene can be expressed from transcription units inserted into DNA or RNA vectors (see, e.g., Couture, A, et al., TIG. (1996), 12:5-10; WO 00/22113, WO 00/22114, and US 6,054,299). Expression is preferably sustained (months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., (1995) Proc. Natl. Acad. Sci. USA 92: 1292).

The individual strand or strands of an RNAi agent can be transcribed from a promoter on an expression vector. Where two separate strands are to be expressed to generate, for example, a dsRNA, two separate expression vectors can be co-introduced (e.g., by transfection or infection) into a target cell. Alternatively, each individual strand of a dsRNA can be transcribed by promoters both of which are located on the same expression plasmid. In one embodiment, a dsRNA is expressed as inverted repeat polynucleotides joined by a linker polynucleotide sequence such that the dsRNA has a stem and loop structure.

RNAi agent expression vectors are generally DNA plasmids or viral vectors. Expression vectors compatible with eukaryotic cells, preferably those compatible with vertebrate cells, can be used to produce recombinant constructs for the expression of an RNAi agent as described herein. Delivery of RNAi agent expressing vectors can be systemic, such as by intravenous or intramuscular administration, by administration to target cells ex-planted from the patient followed by reintroduction into the patient, or by any other means that allows for introduction into a desired target cell.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno- associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells’ genome. The constructs can include viral sequences for transfection, if desired. Alternatively, the construct can be incorporated into vectors capable of episomal replication, e.g. EPV and EBV vectors. Constructs for the recombinant expression of an RNAi agent will generally require regulatory elements, e.g., promoters, enhancers, etc., to ensure the expression of the RNAi agent in target cells. Other aspects to consider for vectors and constructs are known in the art.

### VI. Pharmaceutical Compositions of the Invention

The present disclosure also includes pharmaceutical compositions and formulations which include the RNAi agents of the disclosure. In one embodiment, provided herein are pharmaceutical compositions containing an RNAi agent, as described herein, and a pharmaceutically acceptable carrier. The pharmaceutical compositions containing the RNAi agent are useful for treating a disease or condition treatable by reduction or inhibition of the expression or activity of ALK, e.g., ALK-associated disease, such as type 2 diabetes, obesity, and obesity-associated disease.

In some embodiments, the pharmaceutical compositions of the invention are sterile. In another embodiment, the pharmaceutical compositions of the invention are pyrogen free.

Such pharmaceutical compositions are formulated based on the mode of delivery. One example is compositions that are formulated for systemic administration via parenteral delivery, e.g., by intravenous (IV), intramuscular (IM), or for subcutaneous (subQ) delivery. Another example is compositions that are formulated for direct delivery into the CNS, e.g., by intrathecal or intravitreal routes of injection, optionally by infusion into the brain (e.g., striatum), such as by continuous pump infusion.

The pharmaceutical compositions of the disclosure may be administered in dosages sufficient to inhibit expression of an ALK gene. In general, a suitable dose of an RNAi agent of the disclosure will be in the range of about 0.001 to about 200.0 milligrams per kilogram body weight of the recipient per day, generally in the range of about 1 to 50 mg per kilogram body weight per day.

A repeat-dose regimen may include administration of a therapeutic amount of an RNAi agent on a regular basis, such as monthly to once every six months. In certain embodiments, the RNAi agent is administered about once per quarter (i.e., about once every three months) to about twice per year.

After an initial treatment regimen (e.g., loading dose), the treatments can be administered on a less frequent basis.

In other embodiments, a single dose of the pharmaceutical compositions can be long lasting, such that subsequent doses are administered at not more than 1, 2, 3, or 4 or more month intervals. In some embodiments of the disclosure, a single dose of the pharmaceutical compositions of the disclosure is administered once per month. In other embodiments of the disclosure, a single dose of the pharmaceutical compositions of the disclosure is administered once per quarter to twice per year.

The skilled artisan will appreciate that certain factors can influence the dosage and timing required to effectively treat a subject, including but not limited to the severity of the disease or disorder, previous treatments, the general health or age of the subject, and other diseases present. Moreover, treatment of a subject with a therapeutically effective amount of a composition can include a single treatment or a series of treatments.

Advances in genetics have generated a number of rodent models for the study of various human diseases, including type 2 diabetes, obesity, and obesity-associated disorders that would benefit from reduction in the expression of ALK. Such models can be used for in vivo testing of RNAi agents, as well as for determining a therapeutically effective dose. Suitable rodent models are known in the art and include, for example, those described in, for example, Lutz and Woods (2012) Curr. Protoc. Pharmacol. Chapter: Unit 5.61; and Barrett, et al., (2016) Disease Models and Mechanisms, 9:1245-55. The pharmaceutical compositions of the present disclosure can be administered in a number of ways depending upon whether local or systemic treatment is desired and upon the area to be treated. Administration can be topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration.

The RNAi agents can be delivered in a manner to target a particular tissue, such as the CNS (e.g., neuronal, glial or vascular tissue of the brain).

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful. Suitable topical formulations include those in which the RNAi agents featured in the disclosure are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). RNAi agents featured in the disclosure can be encapsulated within liposomes or can form complexes thereto, in particular to cationic liposomes. Alternatively, RNAi agents can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride or pharmaceutically acceptable salt thereof. Topical formulations are described in detail in US 6,747,014, which is incorporated herein by reference.

### A. RNAi Agent Formulations Comprising Membranous Molecular Assemblies

An RNAi agent for use in the compositions and methods of the disclosure can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the RNAi agent composition. The lipophilic material isolates the aqueous interior from an aqueous exterior, which typically does not include the RNAi agent composition, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the RNAi agent are delivered into the cell where the RNAi agent can specifically bind to a target RNA and can mediate RNAi. In some cases, the liposomes are also specifically targeted, e.g., to direct the RNAi agent to particular cell types.

A liposome containing an RNAi agent can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The RNAi agent preparation is then added to the micelles that include the lipid component. The cationic groups on the lipid interact with the RNAi agent and condense around the RNAi agent to form a liposome. After condensation, the detergent is removed, e.g., by dialysis, to yield a liposomal preparation of RNAi agent.

If necessary a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Methods for producing stable polynucleotide delivery vehicles, which incorporate a polynucleotide/cationic lipid complex as structural components of the delivery vehicle, are further described in, e.g., WO 96/37194, the entire contents of which are incorporated herein by reference. Liposome formation can also include one or more aspects of exemplary methods described in Felgner, P. L. et al., (1987) Proc. Natl. Acad. Sci. USA 8:7413-7417; U.S. Pat. No. 4,897,355; U.S. Pat. No. 5,171,678; Bangham et al., (1965) M. Mol. Biol. 23:238; Olson et al., (1979) Biochim. Biophys. Acta 557:9; Szoka et al., (1978) Proc. Natl. Acad. Sci. 75: 4194; Mayhew et al., (1984) Biochim. Biophys. Acta 775:169; Kim et al., (1983) Biochim. Biophys. Acta 728:339; and Fukunaga et al., (1984) Endocrinol. 115:757. Commonly used techniques for preparing lipid aggregates of appropriate size for use as delivery vehicles include sonication and freeze-thaw plus extrusion (see, e.g., Mayer et al., (1986) Biochim. Biophys. Acta 858:161. Microfluidization can be used when consistently small (50 to 200 nm) and relatively uniform aggregates are desired (Mayhew et al., (1984) Biochim. Biophys. Acta 775:169. These methods are readily adapted to packaging RNAi agent preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes, which are pH-sensitive or negatively charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid or phosphatidylcholine or cholesterol.

Examples of other methods to introduce liposomes into cells in vitro and in vivo include U.S. Pat. No. 5,283,185; U.S. Pat. No. 5,171,678; WO 94/00569; WO 93/24640; WO 91/16024; Felgner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al., (1994) S.T.P.Pharma. Sci., 4(6):466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223:42; Wu et al., (1993) Cancer Research, 53:3765).

Various liposomes comprising one or more glycolipids are known in the art. Papahadjopoulos et al. (Ann. N.Y. Acad. Sci., (1987), 507:64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., (1988), 85:6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver RNAi agents to macrophages.

Further advantages of liposomes include: liposomes obtained from natural phospholipids are biocompatible and biodegradable; liposomes can incorporate a wide range of water and lipid soluble drugs; liposomes can protect encapsulated RNAi agents in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of RNAi agent (see, e.g., Felgner, P. L. et al., (1987) Proc. Natl. Acad. Sci. USA 8:7413-7417, and U.S. Pat. No.4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wisconsin) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., (1991) Biochim. Biophys. Res. Commun. 179:280). Lipopolylysine, made by conjugating polylysine to DOPE, has been reported to be effective for transfection in the presence of serum (Zhou, X. et al., (1991) Biochim. Biophys. Acta 1065:8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, California) and Lipofectamine (DOSPA) (Life Technology, Inc., Gaithersburg, Maryland). Other cationic lipids suitable for the delivery of oligonucleotides are described in WO 98/39359 and WO 96/37194.

Liposomal formulations are particularly suited for topical administration; liposomes present several advantages over other formulations. Such advantages include reduced side effects related to high systemic absorption of the administered drug, increased accumulation of the administered drug at the desired target, and the ability to administer RNAi agent into the skin. In some implementations, liposomes are used for delivering RNAi agent to epidermal cells and also to enhance the penetration of RNAi agent into dermal tissues, e.g., into skin. For example, the liposomes can be applied topically. Topical delivery of drugs formulated as liposomes to the skin has been documented (see, e.g., Weiner et al., (1992) Journal of Drug Targeting, vol. 2,405-410 and du Plessis et al., (1992) Antiviral Research, 18:259-265; Mannino, R. J. and Fould-Fogerite, S., (1998) Biotechniques 6:682-690; Itani, T. et al., (1987) Gene 56:267-276; Nicolau, C. et al. (1987) Meth. Enzymol. 149:157-176; Straubinger, R. M. and Papahadjopoulos, D. (1983) Meth. Enzymol. 101:512-527; Wang, C. Y. and Huang, L., (1987) Proc. Natl. Acad. Sci. USA 84:7851-7855).

Non-ionic liposomal systems have also been examined to determine their utility in the delivery of drugs to the skin, in particular systems comprising non-ionic surfactant and cholesterol. Non-ionic liposomal formulations comprising Novasome I (glyceryl dilaurate/cholesterol/polyoxyethylene-10-stearyl ether) and Novasome II (glyceryl distearate/ cholesterol/polyoxyethylene-10-stearyl ether) were used to deliver a drug into the dermis of mouse skin. Such formulations with RNAi agent are useful for treating a dermatological disorder.

Liposomes that include RNAi agents can be made highly deformable. Such deformability can enable the liposomes to penetrate through pore that are smaller than the average radius of the liposome. For example, transfersomes are a type of deformable liposomes. Transferosomes can be made by adding surface edge activators, usually surfactants, to a standard liposomal composition. Transfersomes that include RNAi agent can be delivered, for example, subcutaneously by infection in order to deliver RNAi agent to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transferosomes can be self-optimizing (adaptive to the shape of pores, e.g., in the skin), self-repairing, and can frequently reach their targets without fragmenting, and often self-loading.

Other formulations amenable to the present disclosure are described in PCT application number PCT/US2008/088588, filed Dec. 13, 2008; PCT/US2008/088587, filed Dec. 13, 2008; PCT/US2009/041442, filed Apr. 22, 2009; and PCT/US2007/080331, filed Oct. 3, 2007.

Transfersomes, yet another type of liposomes, are highly deformable lipid aggregates which are attractive candidates for drug delivery vehicles. Transfersomes can be described as lipid droplets which are so highly deformable that they are easily able to penetrate through pores which are smaller than the droplet. Transfersomes are adaptable to the environment in which they are used, e.g., they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as those described herein, particularly in emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides. The use of surfactants in drug products, formulations and in emulsions has been reviewed (Rieger, in Pharmaceutical Dosage Forms, Marcel Dekker, Inc., New York, N.Y., 1988, p. 285).

The RNAi agent for use in the methods of the disclosure can also be provided as micellar formulations. “Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

A mixed micellar formulation suitable for delivery through transdermal membranes may be prepared by mixing an aqueous solution of the siRNA composition, an alkali metal Cs to C₂₂ alkyl sulphate, and a micelle forming compounds. Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerin, polyglycerin, lysine, polylysine, triolein, polyoxyethylene ethers and analogues thereof, polidocanol alkyl ethers and analogues thereof, chenodeoxycholate, deoxycholate, and mixtures thereof. The micelle forming compounds may be added at the same time or after addition of the alkali metal alkyl sulphate. Mixed micelles will form with substantially any kind of mixing of the ingredients but vigorous mixing in order to provide smaller size micelles.

In one method a first micellar composition is prepared which contains the siRNA composition and at least the alkali metal alkyl sulphate. The first micellar composition is then mixed with at least three micelle forming compounds to form a mixed micellar composition. In another method, the micellar composition is prepared by mixing the siRNA composition, the alkali metal alkyl sulphate and at least one of the micelle forming compounds, followed by addition of the remaining micelle forming compounds, with vigorous mixing.

Phenol or m-cresol may be added to the mixed micellar composition to stabilize the formulation and protect against bacterial growth. Alternatively, phenol or m-cresol may be added with the micelle forming ingredients. An isotonic agent such as glycerin may also be added after formation of the mixed micellar composition.

For delivery of the micellar formulation as a spray, the formulation can be put into an aerosol dispenser and the dispenser is charged with a propellant. The propellant, which is under pressure, is in liquid form in the dispenser. The ratios of the ingredients are adjusted so that the aqueous and propellant phases become one, i.e., there is one phase. If there are two phases, it is necessary to shake the dispenser prior to dispensing a portion of the contents, e.g., through a metered valve. The dispensed dose of pharmaceutical agent is propelled from the metered valve in a fine spray.

Propellants may include hydrogen-containing chlorofluorocarbons, hydrogen-containing fluorocarbons, dimethyl ether and diethyl ether. In certain embodiments, HFA 134a (1,1,1,2 tetrafluoroethane) may be used.

The specific concentrations of the essential ingredients can be determined by relatively straightforward experimentation. For absorption through the oral cavities, it is often desirable to increase, e.g., at least double or triple, the dosage for through injection or administration through the gastrointestinal tract.

### B. Lipid Particles

RNAi agents, e.g., dsRNAs of in the disclosure may be fully encapsulated in a lipid formulation, e.g., a LNP, or other nucleic acid-lipid particle.

As used herein, the term “LNP” refers to a stable nucleic acid-lipid particle. LNPs typically contain a cationic lipid, a non-cationic lipid, and a lipid that prevents aggregation of the particle (e.g., a PEG-lipid conjugate). LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in WO 00/03683. The particles of the present disclosure typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid- lipid particles of the present disclosure are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Pat. Publication No. 2010/0324120 and WO 96/40964.

In one embodiment, the lipid to drug ratio (mass/mass ratio) (e.g., lipid to dsRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the disclosure.

Certain specific LNP formulations for delivery of RNAi agents have been described in the art, including, e.g., “LNP01” formulations as described in, e.g., WO 2008/042973, which is hereby incorporated by reference.

Additional exemplary lipid-dsRNA formulations are identified in the table below.

| | | |
|---|---|---|
| | Ionizable/Cationic Lipid | cationic lipid/non-cationic lipid/cholesterol/PEG-lipid conjugate Lipid:siRNA ratio |
| SNALP-1 | 1,2-Dilinolenyloxy-N,N-dimethylaminopropane (DLinDMA) | DLinDMA/DPPC/Cholesterol/PEG-cDMA (57.1/7.1/34.4/1.4) lipid:siRNA ~ 7:1 |
| 2-XTC | 2,2-Dilinoleyl-4-dimethylaminoethyl-[1,3]-dioxolane (XTC) | XTC/DPPC/Cholesterol/PEG-cDMA 57.1/7.1/34.4/1.4 lipid:siRNA ~ 7:1 |
| LNP05 | 2,2-Dilinoleyl-4-dimethylaminoethyl-[1,3]-dioxolane (XTC) | XTC/DSPC/Cholesterol/PEG-DMG 57.5/7.5/31.5/3.5 lipid:siRNA ~ 6:1 |
| LNP06 | 2,2-Dilinoleyl-4-dimethylaminoethyl-[1,3]-dioxolane (XTC) | XTC/DSPC/Cholesterol/PEG-DMG 57.5/7.5/31.5/3.5 lipid:siRNA ~ 11:1 |
| LNP07 | 2,2-Dilinoleyl-4-dimethylaminoethyl-[1,3]-dioxolane (XTC) | XTC/DSPC/Cholesterol/PEG-DMG 60/7.5/31/1.5, lipid:siRNA ~ 6:1 |
| LNP08 | 2,2-Dilinoleyl-4-dimethylaminoethyl-[1,3]-dioxolane (XTC) | XTC/DSPC/Cholesterol/PEG-DMG 60/7.5/31/1.5, lipid:siRNA ~ 11:1 |
| LNP09 | 2,2-Dilinoleyl-4-dimethylaminoethyl-[1,3]-dioxolane (XTC) | XTC/DSPC/Cholesterol/PEG-DMG 50/10/38.5/1.5 Lipid:siRNA 10:1 |
| LNP 10 | (3aR,5s,6aS)-N,N-dimethyl-2,2-di((9Z,12Z)-octadeca-9,12-dienyl)tetrahydro-3aH-cyclopenta[d][1,3]dioxol-5-amine (ALN100) | ALN 100/DSPC/Cholesterol/PEG-DMG 50/10/38.5/1.5 Lipid:siRNA 10:1 |
| LNP11 | (6Z,9Z,28Z,31Z)-heptatriaconta-6,9,28,31-tetraen-19-yl 4-(dimethylamino)butanoate (MC3) | MC-3/DSPC/Cholesterol/PEG-DMG 50/10/38.5/1.5 Lipid:siRNA 10:1 |
| LNP12 | 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (Tech G1) | Tech G1/DSPC/Cholesterol/PEG-DMG 50/10/38.5/1.5 Lipid:siRNA 10:1 |
| LNP13 | XTC | XTC/DSPC/Chol/PEG-DMG 50/10/38.5/1.5 Lipid:siRNA: 33:1 |
| LNP14 | MC3 | MC3/DSPC/Chol/PEG-DMG 40/15/40/5 Lipid: siRNA: 11:1 |
| LNP15 | MC3 | MC3/DSPC/Chol/PEG-DSG/GalNAc-PEG-DSG 50/10/35/4.5/0.5 Lipid: siRNA: 11:1 |
| LNP16 | MC3 | MC3/DSPC/Chol/PEG-DMG 50/10/38.5/1.5 Lipid:siRNA: 7:1 |
| LNP17 | MC3 | MC3/DSPC/Chol/PEG-DSG 50/10/38.5/1.5 Lipid:siRNA: 10:1 |
| LNP18 | MC3 | MC3/DSPC/Chol/PEG-DMG 50/10/38.5/1.5 Lipid:siRNA: 12:1 |
| LNP19 | MC3 | MC3/DSPC/Chol/PEG-DMG 50/10/35/5 Lipid:siRNA: 8:1 |
| LNP20 | MC3 | MC3/DSPC/Chol/PEG-DPG 50/10/38.5/1.5 Lipid:siRNA: 10:1 |
| LNP21 | C12-200 | C12-200/DSPC/Chol/PEG-DSG 50/10/38.5/1.5 Lipid:siRNA: 7:1 |
| LNP22 | XTC | XTC/DSPC/Chol/PEG-DSG 50/10/38.5/1.5 Lipid:siRNA: 10:1 |

DSPC: distearoylphosphatidylcholine
DPPC: dipalmitoylphosphatidylcholine
PEG-DMG: PEG-didimyristoyl glycerol (C14-PEG, or PEG-C14) (PEG with avg mol wt of 2000)
PEG-DSG: PEG-distyryl glycerol (C18-PEG, or PEG-C18) (PEG with avg mol wt of 2000)
PEG-cDMA: PEG-carbamoyl-1,2-dimyristyloxypropylamine (PEG with avg mol wt of 2000)

SNALP (1,2-Dilinolenyloxy-N,N-dimethylaminopropane (DLinDMA)) comprising formulations are described in WO 2009/127060, which is hereby incorporated by reference.

XTC comprising formulations are described in WO 2010/088537, the entire contents of which are hereby incorporated herein by reference.

MC3 comprising formulations are described, e.g., in U.S. Pat. Publication No. 2010/0324120, the entire contents of which are hereby incorporated by reference.

ALNY-100 comprising formulations are described in WO 2010/054406, the entire contents of which are hereby incorporated herein by reference.

C12-200 comprising formulations are described in WO 2010/129709, the entire contents of which are hereby incorporated herein by reference.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders can be desirable. In some embodiments, oral formulations are those in which dsRNAs featured in the disclosure are administered in conjunction with one or more penetration enhancer surfactants and chelators. Suitable surfactants include fatty acids or esters or salts thereof, bile acids or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether. DsRNAs featured in the disclosure can be delivered orally, in granular form including sprayed dried particles, or complexed to form micro or nanoparticles. DsRNA complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Suitable complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g., p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for dsRNAs and their preparation are described in detail in U.S. Pat. 6,887,906, U.S. 2003/0027780, and U.S. Pat. No. 6,747,014, each of which is incorporated herein by reference.

Compositions and formulations for parenteral, intraparenchymal (into the brain), intrathecal, intraventricular or intrahepatic administration can include sterile aqueous solutions which can also contain buffers, diluents and other suitable additives such as, but not limited to, penetration enhancers, carrier compounds and other pharmaceutically acceptable carriers or excipients.

Pharmaceutical compositions of the present disclosure include, but are not limited to, solutions, emulsions, and liposome-containing formulations. These compositions can be generated from a variety of components that include, but are not limited to, preformed liquids, self-emulsifying solids and self-emulsifying semisolids. Exemplary formulations include those that target the brain when treating or preventing ALK-associated diseases or disorders.

The pharmaceutical formulations of the present disclosure, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present disclosure can be formulated into any of many possible dosage forms such as, but not limited to, tablets, capsules, gel capsules, liquid syrups, soft gels, suppositories, and enemas. The compositions of the present disclosure can also be formulated as suspensions in aqueous, non-aqueous or mixed media. Aqueous suspensions can further contain substances which increase the viscosity of the suspension including, for example, sodium carboxymethylcellulose, sorbitol or dextran. The suspension can also contain stabilizers.

### C. Additional Formulations

### I. Emulsions

The compositions of the present disclosure can be prepared and formulated as emulsions. Emulsions are typically heterogeneous systems of one liquid dispersed in another in the form of droplets usually exceeding 0.1 µm in diameter (see e.g., Ansel’s Pharmaceutical Dosage Forms and Drug Delivery Systems, Allen, LV., Popovich NG., and Ansel HC., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington’s Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the active drug which can be present as a solution in either aqueous phase, oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise, a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion can be a semisolid or a solid, as is the case of emulsion-style ointment bases and creams. Other means of stabilizing emulsions entail the use of emulsifiers that can be incorporated into either phase of the emulsion. Emulsifiers can broadly be classified into four categories: synthetic surfactants, naturally occurring emulsifiers, absorption bases, and finely dispersed solids (see e.g., Ansel’s Pharmaceutical Dosage Forms and Drug Delivery Systems, Allen, LV., Popovich NG., and Ansel HC., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (see e.g., Ansel’s Pharmaceutical Dosage Forms and Drug Delivery Systems, Allen, LV., Popovich NG., and Ansel HC., 2004, Lippincott Williams & Wilkins (8th ed.), New York, NY; Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic and amphoteric (see e.g., Ansel’s Pharmaceutical Dosage Forms and Drug Delivery Systems, Allen, LV., Popovich NG., and Ansel HC., 2004, Lippincott Williams & Wilkins (8th ed.), New York, NY Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that can readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (see e.g., Ansel’s Pharmaceutical Dosage Forms and Drug Delivery Systems, Allen, LV., Popovich NG., and Ansel HC., 2004, Lippincott Williams & Wilkins (8th ed.), New York, NY; Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (see e.g., Ansel’s Pharmaceutical Dosage Forms and Drug Delivery Systems, Allen, LV., Popovich NG., and Ansel HC., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

### II. Microemulsions

In one embodiment of the present disclosure, the compositions of RNAi agents and nucleic acids are formulated as microemulsions. A microemulsion can be defined as a system of water, oil and amphiphile which is a single optically isotropic and thermodynamically stable liquid solution (see e.g., Ansel’s Pharmaceutical Dosage Forms and Drug Delivery Systems, Allen, LV., Popovich NG., and Ansel HC., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245). Typically, microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used, and on the structure and geometric packing of the polar heads and hydrocarbon tails of the surfactant molecules (Schott, in Remington’s Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (see e.g., Ansel’s Pharmaceutical Dosage Forms and Drug Delivery Systems, Allen, LV., Popovich NG., and Ansel HC., 2004, Lippincott Williams & Wilkins (8th ed.), New York, NY; Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (SO750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions can, however, be prepared without the use of cosurfactants and alcohol-free self-emulsifying microemulsion systems are known in the art. The aqueous phase can typically be, but is not limited to, water, an aqueous solution of the drug, glycerol, PEG300, PEG400, polyglycerols, propylene glycols, and derivatives of ethylene glycol. The oil phase can include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (see e.g., U.S. Pat. Nos. 6,191,105; 7,063,860; 7,070,802; 7,157,099; Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (see e.g., U.S. Pat. Nos. 6,191,105; 7,063,860; 7,070,802; 7,157,099; Constantinides et al., Pharmaceutical Research, 1994, 11, 1385; Ho et al., J. Pharm. Sci., 1996, 85, 138-143). Often microemulsions can form spontaneously when their components are brought together at ambient temperature. This can be particularly advantageous when formulating thermolabile drugs, peptides or RNAi agents. Microemulsions have also been effective in the transdermal delivery of active components in both cosmetic and pharmaceutical applications. It is expected that the microemulsion compositions and formulations of the present disclosure will facilitate the increased systemic absorption of RNAi agents and nucleic acids from the gastrointestinal tract, as well as improve the local cellular uptake of RNAi agents and nucleic acids.

Microemulsions of the present disclosure can also contain additional components and additives such as sorbitan monostearate (Grill 3), Labrasol, and penetration enhancers to improve the properties of the formulation and to enhance the absorption of the RNAi agents and nucleic acids of the present disclosure. Penetration enhancers used in the microemulsions of the present disclosure can be classified as belonging to one of five broad categories--surfactants, fatty acids, bile salts, chelating agents, and non-chelating non-surfactants (Lee et al., Critical Reviews in Therapeutic Drug Carrier Systems, 1991, p. 92). Each of these classes has been discussed above.

### III. Microparticles

An RNAi agent of the disclosure may be incorporated into a particle, e.g., a microparticle. Microparticles can be produced by spray-drying, but may also be produced by other methods including lyophilization, evaporation, fluid bed drying, vacuum drying, or a combination of these techniques.

### IV. Penetration Enhancers

In one embodiment, the present disclosure employs various penetration enhancers to effect the efficient delivery of nucleic acids, particularly RNAi agents, to the skin of animals. Most drugs are present in solution in both ionized and nonionized forms. However, usually only lipid soluble or lipophilic drugs readily cross cell membranes. It has been discovered that even non-lipophilic drugs can cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers can be classified as belonging to one of five broad categories, i.e., surfactants, fatty acids, bile salts, chelating agents, and non-chelating non-surfactants (see e.g., Malmsten, M. Surfactants and polymers in drug delivery, Informa Health Care, New York, NY, 2002; Lee et al., Critical Reviews in Therapeutic Drug Carrier Systems, 1991, p.92). Each of the above mentioned classes of penetration enhancers are described below in greater detail.

Surfactants (or “surface-active agents”) are chemical entities which, when dissolved in an aqueous solution, reduce the surface tension of the solution or the interfacial tension between the aqueous solution and another liquid, with the result that absorption of RNAi agents through the mucosa is enhanced. In addition to bile salts and fatty acids, these penetration enhancers include, for example, sodium lauryl sulfate, polyoxyethylene-9-lauryl ether and polyoxyethylene-20-cetyl ether) (see e.g., Malmsten, M. Surfactants and polymers in drug delivery, Informa Health Care, New York, NY, 2002; Lee et al., Critical Reviews in Therapeutic Drug Carrier Systems, 1991, p.92); and perfluorochemical emulsions, such as FC-43. Takahashi et al., J. Pharm. Pharmacol., 1988, 40, 252).

Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₂₀ alkyl esters thereof (e.g., methyl, isopropyl and t-butyl), and mono-and di-glycerides thereof (i.e., oleate, laurate, caprate, myristate, palmitate, stearate, linoleate, etc.) (see e.g., Touitou, E., et al. Enhancement in Drug Delivery, CRC Press, Danvers, MA, 2006; Lee et al., Critical Reviews in Therapeutic Drug Carrier Systems, 1991, p.92; Muranishi, Critical Reviews in Therapeutic Drug Carrier Systems, 1990, 7, 1-33; El Hariri et al., J. Pharm. Pharmacol., 1992, 44, 651-654).

The physiological role of bile includes the facilitation of dispersion and absorption of lipids and fat-soluble vitamins (see e.g., Malmsten, M. Surfactants and polymers in drug delivery, Informa Health Care, New York, NY, 2002; Brunton, Chapter 38 in: Goodman & Gilman’s The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. Suitable bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (see e.g., Malmsten, M. Surfactants and polymers in drug delivery, Informa Health Care, New York, NY, 2002; Lee et al., Critical Reviews in Therapeutic Drug Carrier Systems, 1991, page 92; Swinyard, Chapter 39 In: Remington’s Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating agents, as used in connection with the present disclosure, can be defined as compounds that remove metallic ions from solution by forming complexes therewith, with the result that absorption of RNAi agents through the mucosa is enhanced. With regards to their use as penetration enhancers in the present disclosure, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Suitable chelating agents include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(see e.g., Katdare, A. et al., Excipient development for pharmaceutical, biotechnology, and drug delivery, CRC Press, Danvers, MA, 2006; Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

As used herein, non-chelating non-surfactant penetration enhancing compounds can be defined as compounds that demonstrate insignificant activity as chelating agents or as surfactants but that nonetheless enhance absorption of RNAi agents through the alimentary mucosa (see e.g., Muranishi, Critical Reviews in Therapeutic Drug Carrier Systems, 1990, 7, 1-33). This class of penetration enhancers includes, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of RNAi agents at the cellular level can also be added to the pharmaceutical and other compositions of the present disclosure. For example, cationic lipids, such as lipofectin (Junichi et al, U.S. Pat. No. 5,705,188), cationic glycerol derivatives, and polycationic molecules, such as polylysine (WO 97/30731), are also known to enhance the cellular uptake of dsRNAs.

Other agents can be utilized to enhance the penetration of the administered nucleic acids, including glycols such as ethylene glycol and propylene glycol, pyrrols such as 2-pyrrol, azones, and terpenes such as limonene and menthone.

### VI. Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipients suitable for non-parenteral administration which do not deleteriously react with nucleic acids can also be used to formulate the compositions of the present disclosure. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

### VII. Other Components

The compositions of the present disclosure can additionally contain other adjunct components conventionally found in pharmaceutical compositions, at their art-established usage levels. Thus, for example, the compositions can contain additional, compatible, pharmaceutically-active materials such as, for example, antipruritics, astringents, local anesthetics or anti-inflammatory agents, or can contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings or aromatic substances and the like which do not deleteriously interact with the nucleic acid(s) of the formulation.

Aqueous suspensions can contain substances which increase the viscosity of the suspension including, for example, sodium carboxymethylcellulose, sorbitol or dextran. The suspension can also contain stabilizers.

In some embodiments, pharmaceutical compositions featured in the disclosure include (a) one or more RNAi agents and (b) one or more agents which function by a non-RNAi mechanism and which are useful in treating an ALK-associated disorder, e.g., type 2 diabetes or obesity. Examples of such agents include, but are not limited to, orlistat (Alli, Xenical), phentermine and topiramate (Qsymia), bupropion and naltrexone (Contrave), liraglutide (Saxenda, Victoza), and agents that decrease or otherwise affect the ALK activity.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred.

The data obtained from cell culture assays and animal studies can be used in formulating a range of dosage for use in humans. The dosage of compositions featured herein in the disclosure lies generally within a range of circulating concentrations that include the ED₅₀ with little or no toxicity. The dosage can vary within this range depending upon the dosage form employed and the route of administration utilized. For any compound used in the methods featured in the disclosure, the therapeutically effective dose can be estimated initially from cell culture assays. A dose can be formulated in animal models to achieve a circulating plasma concentration range of the compound or, when appropriate, of the polypeptide product of a target sequence (e.g., achieving a decreased concentration of the polypeptide) that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

In addition to their administration, as discussed above, the RNAi agents featured in the disclosure can be administered in combination with other known agents effective in treatment of pathological processes mediated by nucleotide repeat expression. In any event, the administering physician can adjust the amount and timing of RNAi agent administration on the basis of results observed using standard measures of efficacy known in the art or described herein.

### VII. Kits

In certain aspects, the instant disclosure provides kits that include a suitable container containing a pharmaceutical formulation of a siRNA compound, e.g., a double-stranded siRNA compound, or ssiRNA compound, (e.g., a precursor, e.g., a larger siRNA compound which can be processed into a ssiRNA compound, or a DNA which encodes an siRNA compound, e.g., a double-stranded siRNA compound, or ssiRNA compound, or precursor thereof).

Such kits include one or more dsRNA agent(s) and instructions for use, e.g., instructions for administering a prophylactically or therapeutically effective amount of a dsRNA agent(s). The dsRNA agent may be in a vial or a pre-filled syringe. The kits may optionally further comprise means for administering the dsRNA agent (e.g., an injection device, such as a pre-filled syringe or an intrathecal pump), or means for measuring the inhibition of C3 (e.g., means for measuring the inhibition of ALK mRNA, ALK protein, and/or ALK activity). Such means for measuring the inhibition of ALK may comprise a means for obtaining a sample from a subject, such as, e.g., a CSF and/or plasma sample. The kits of the invention may optionally further comprise means for determining the therapeutically effective or prophylactically effective amount.

In certain embodiments the individual components of the pharmaceutical formulation may be provided in one container. Alternatively, it may be desirable to provide the components of the pharmaceutical formulation separately in two or more containers, e.g., one container for a siRNA compound preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

### VIII. Methods for Inhibiting ALK Expression

The present disclosure also provides methods of inhibiting expression of an ALK gene in a cell. The methods include contacting a cell with an RNAi agent, e.g., double stranded RNAi agent, in an amount effective to inhibit expression and/or activity of ALK in the cell, thereby inhibiting expression and/or activity of ALK in the cell. In certain embodiments of the disclosure, ALK expression and/or activity is inhibited by at least 30% preferentially in CNS (e.g., brain) cells. In specific embodiments, ALK expression and/or activity is inhibited by at least 30%. In other embodiments of the disclosure, ALK expression and/or activity is inhibited preferentially by at least 30% in endocrine (e.g., pituitary gland, adrenal gland) cells.

Contacting of a cell with an RNAi agent, e.g., a double stranded RNAi agent, may be done in vitro or in vivo. Contacting a cell in vivo with the RNAi agent includes contacting a cell or group of cells within a subject, e.g., a human subject, with the RNAi agent. Combinations of in vitro and in vivo methods of contacting a cell are also possible.

Contacting a cell may be direct or indirect, as discussed above. Furthermore, contacting a cell may be accomplished via a targeting ligand, including any ligand described herein or known in the art. In some embodiments, the targeting ligand is a carbohydrate moiety, e.g., a GalNAc ligand, or any other ligand that directs the RNAi agent to a site of interest.

The term “inhibiting,” as used herein, is used interchangeably with “reducing,” “silencing,” “downregulating,” “suppressing” and other similar terms, and includes any level of inhibition. In certain embodiments, a level of inhibition, e.g., for an RNAi agent of the instant disclosure, can be assessed in cell culture conditions, e.g., wherein cells in cell culture are transfected via Lipofectamine™-mediated transfection at a concentration in the vicinity of a cell of 10 nM or less, 1 nM or less, etc. Knockdown of a given RNAi agent can be determined via comparison of pre-treated levels in cell culture versus post-treated levels in cell culture, optionally also comparing against cells treated in parallel with a scrambled or other form of control RNAi agent. Knockdown in cell culture of, e.g., at least about 30%, can thereby be identified as indicative of “inhibiting” or “reducing”, “downregulating” or “suppressing”, etc. having occurred. It is expressly contemplated that assessment of targeted mRNA or encoded protein levels (and therefore an extent of “inhibiting”, etc. caused by an RNAi agent of the disclosure) can also be assessed in in vivo systems for the RNAi agents of the instant disclosure, under properly controlled conditions as described in the art.

The phrase “inhibiting ALK,” “inhibiting expression of an ALK gene” or “inhibiting expression of ALK,” as used herein, includes inhibition of expression of any ALK gene (such as, e.g., a mouse ALK gene, a rat ALK gene, a monkey ALK gene, or a human ALK gene) as well as variants or mutants of an ALK gene that encode an ALK protein. Thus, the ALK gene may be a wild-type ALK gene, a mutant ALK gene, or a transgenic ALK gene in the context of a genetically manipulated cell, group of cells, or organism.

“Inhibiting expression of an ALK gene” includes any level of inhibition of an ALK gene, e.g., at least partial suppression of the expression of an ALK gene, such as an inhibition by at least 30%. In certain embodiments, inhibition is by at least 35%, at least 40%, at least 45%, by at least 50%, at least 55%, by at least 60%, by at least 65%, by at least 70%, by at least 75%, by at least 80%, by at least 85%, by at least 90%, by at least 95%, or by at least 99%. ALK inhibition can be measured using the in vitro assay with, e.g., A549 cells and a 10 nM concentration of the RNA agent and the PCR assay as provided in the examples herein, are contemplated to be within the scope of the present disclosure. In some embodiments, ALK inhibition can be measured using the in vitro assay with primary mouse hepatocytes. In another embodiment, ALK inhibition can be measured using the in vitro assay with Cos-7 (Dual-Luciferase psiCHECK2 vector). In yet another embodiment, ALK inhibition can be measured using the in vitro assay with BE(2)-C cells. In some embodiments, ALK inhibition can be measured using the in vitro assay with Neuro-2a cells.

The expression of an ALK gene may be assessed based on the level of any variable associated with ALK gene expression, e.g., ALK mRNA level (e.g., sense mRNA, antisense mRNA, and/or total ALK mRNA) or ALK protein level (e.g., total ALK protein and/or wild-type ALK protein).

Inhibition may be assessed by a decrease in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

For example, in some embodiments of the methods of the disclosure, expression of an ALK gene is inhibited by at least 30%, 40%, 50%, 60%, 70%, 80%, 85%, 90%, or 95%, or to below the level of detection of the assay. In certain embodiments, the methods include a clinically relevant inhibition of expression of ALK, e.g. as demonstrated by a clinically relevant outcome after treatment of a subject with an agent to reduce the expression of ALK.

Inhibition of the expression of an ALK gene may be manifested by a reduction of the amount of mRNA expressed by a first cell or group of cells (such cells may be present, for example, in a sample derived from a subject) in which an ALK gene is transcribed and which has or have been treated (e.g., by contacting the cell or cells with an RNAi agent of the disclosure, or by administering an RNAi agent of the disclosure to a subject in which the cells are or were present) such that the expression of an ALK gene is inhibited, as compared to a second cell or group of cells substantially identical to the first cell or group of cells but which has not or have not been so treated (control cell(s) not treated with an RNAi agent or not treated with an RNAi agent targeted to the gene of interest). The degree of inhibition may be expressed in terms of:

$$\frac{\left(\text{mRNA in control cells}\right) \text{-} \left(\text{mRNA in treated cells}\right)}{\left(\text{mRNA in control cells}\right)} \cdot 100 \%$$

In other embodiments, inhibition of the expression of an ALK gene may be assessed in terms of a reduction of a parameter that is functionally linked to an ALK gene expression, e.g., ALK protein expression. ALK gene silencing may be determined in any cell expressing ALK, either endogenous or heterologous from an expression construct, and by any assay known in the art.

Inhibition of the expression of an ALK protein may be manifested by a reduction in the level of the ALK protein (or functional parameter, e.g., kinase activity) that is expressed by a cell or group of cells (e.g., the level of protein expressed in a sample derived from a subject). As explained above, for the assessment of mRNA suppression, the inhibition of protein expression levels in a treated cell or group of cells may similarly be expressed as a percentage of the level of protein in a control cell or group of cells. In some embodiments, the phrase “inhibiting ALK”, can also refer to the inhibition of the kinase activity of ALK, e.g., at least partial suppression of the ALK kinase activity, such as an inhibition by at least 30%. In certain embodiments, inhibition of the ALK kinase activity is by at least 35%, at least 40%, at least 45%, by at least 50%, at least 55%, by at least 60%, by at least 65%, by at least 70%, by at least 75%, by at least 80%, by at least 85%, by at least 90%, by at least 95%, or by at least 99%. ALK kinase activity can be measured using the in vitro assay with, e.g., the assay described in (Smith et al. (2006) Nature Neuroscience 9(10): 1231-3).

A control cell or group of cells that may be used to assess the inhibition of the expression of an ALK gene includes a cell or group of cells that has not yet been contacted with an RNAi agent of the disclosure. For example, the control cell or group of cells may be derived from an individual subject (e.g., a human or animal subject) prior to treatment of the subject with an RNAi agent.

The level of ALK mRNA that is expressed by a cell or group of cells may be determined using any method known in the art for assessing mRNA expression. In one embodiment, the level of expression of ALK in a sample is determined by detecting a transcribed polynucleotide, or portion thereof, e.g., mRNA of the ALK gene. RNA may be extracted from cells using RNA extraction techniques including, for example, using acid phenol/guanidine isothiocyanate extraction (RNAzol B; Biogenesis), RNeasy™ RNA preparation kits (Qiagen®) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Strand specific ALK mRNAs may be detected using the quantitative RT-PCR and or droplet digital PCR methods described in, for example, Jiang, et al. supra, Lagier-Tourenne, et al., supra and Jiang, et al., supra. Circulating ALK mRNA may be detected using methods the described in WO2012/177906, the entire contents of which are hereby incorporated herein by reference.

In some embodiments, the level of expression of ALK is determined using a nucleic acid probe. The term “probe”, as used herein, refers to any molecule that is capable of selectively binding to a specific ALK nucleic acid or protein, or fragment thereof. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses and probe arrays. One method for the determination of mRNA levels involves contacting the isolated mRNA with a nucleic acid molecule (probe) that can hybridize to ALK mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix® gene chip array. A skilled artisan can readily adapt known mRNA detection methods for use in determining the level of ALK mRNA.

An alternative method for determining the level of expression of ALK in a sample involves the process of nucleic acid amplification or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88: 189-193), self-sustained sequence replication (Guatelli et al. (1990) Proc. Natl. Acad. Sci. USA 87: 1874-1878), transcriptional amplification system (Kwoh et al. (1989) Proc. Natl. Acad. Sci. USA 86: 1173-1177), Q-Beta Replicase (Lizardi et al. (1988) Bio/Technology 6: 1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the disclosure, the level of expression of ALK is determined by quantitative fluorogenic RT-PCR (i.e., the TaqMan™ System), by a Dual-Glo® Luciferase assay, or by other art-recognized method for measurement of ALK expression or mRNA level.

The expression level of ALK mRNA may be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The determination of ALK expression level may also comprise using nucleic acid probes in solution.

In some embodiments, the level of mRNA expression is assessed using branched DNA (bDNA) assays or real time PCR (qPCR). The use of this PCR method is described and exemplified in the Examples presented herein. Such methods can also be used for the detection of ALK nucleic acids. The level of ALK protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of ALK proteins. In some embodiments, the efficacy of the methods of the disclosure in the treatment of an ALK-associated disease, e.g., type 2 diabetes or obesity, is assessed by a decrease in ALK mRNA level (e.g., by assessment of a CSF sample and/or plasma sample for ALK level, by brain biopsy, or otherwise).

In some embodiments of the methods of the disclosure, the RNAi agent is administered to a subject such that the RNAi agent is delivered to a specific site within the subject. The inhibition of expression of ALK may be assessed using measurements of the level or change in the level of ALK mRNA (e.g., sense mRNA, antisense mRNA, total ALK mRNA) and/or ALK protein (e.g., total ALK protein, wild-type ALK protein) in a sample derived from a specific site within the subject, e.g., CNS cells. In certain embodiments, the methods include a clinically relevant inhibition of expression of ALK, e.g. as demonstrated by a clinically relevant outcome after treatment of a subject with an agent to reduce the expression of ALK, such as, for example, reduced body weight, reduced body mass index, reduced waist circumference, reduced hip circumference, increased fat-free body mass, increased ratio of fat-free mass to fat mass, reduced fat deposition, decreased triglyceride levels, improved dyslipidemia, improved blood lipid profile, or improved blood glucose levels in a subject. In certain embodiments, a clinically relevant inhibition of expression of ALK is demonstrated by maintenance of body weight, body mass index, waist circumference, hip circumference, fat-free body mass, ratio of fat-free mass to fat mass, fat deposition, triglyceride levels, lipid profile, or glucose levels in a subject.

As used herein, the terms detecting or determining a level of an analyte are understood to mean performing the steps to determine if a material, e.g., protein, RNA, is present. As used herein, methods of detecting or determining include detection or determination of an analyte level that is below the level of detection for the method used.

### IX. Methods of Treating or Preventing ALK-Associated Diseases

The present disclosure also provides methods of using an RNAi agent of the disclosure or a composition containing an RNAi agent of the disclosure to reduce or inhibit ALK expression in a cell. The methods include contacting the cell with a dsRNA of the disclosure and maintaining the cell for a time sufficient to obtain degradation of the mRNA transcript of an ALK gene, thereby inhibiting expression of the ALK gene in the cell.

Reduction in gene expression can be assessed by any methods known in the art. For example, a reduction in the expression of ALK may be determined by determining the mRNA expression level of ALK using methods routine to one of ordinary skill in the art, e.g., northern blotting, qRT-PCR; by determining the protein level of ALK using methods routine to one of ordinary skill in the art, such as western blotting, immunological techniques.

In the methods of the disclosure the cell may be contacted in vitro or in vivo, i.e., the cell may be within a subject.

A cell suitable for treatment using the methods of the disclosure may be any cell that expresses an ALK gene. A cell suitable for use in the methods of the disclosure may be a mammalian cell, e.g., a primate cell (such as a human cell or a non-human primate cell, e.g., a monkey cell or a chimpanzee cell), a non-primate cell (such as a rat cell, or a mouse cell). In one embodiment, the cell is a human cell, e.g., a human CNS cell.

ALK expression (e.g., as assessed by sense mRNA, antisense mRNA, total ALK mRNA, total ALK protein) is inhibited in the cell by at least 30%, 40%, preferably at least 50%, 60%, 70%, 80%, 85%, 90%, or 95%, 99%, or to below the level of detection of the assay.

The in vivo methods of the disclosure may include administering to a subject a composition containing an RNAi agent, where the RNAi agent includes a nucleotide sequence that is complementary to at least a part of an RNA transcript of the ALK gene of the mammal to be treated. When the organism to be treated is a mammal such as a human, the composition can be administered by any means known in the art including, but not limited to oral, intraperitoneal, or parenteral routes, including intracranial (e.g., intraventricular, intraparenchymal, and intrathecal), intravenous, intramuscular, intravitreal, subcutaneous, transdermal, airway (aerosol), nasal, rectal, and topical (including buccal and sublingual) administration. In certain embodiments, the compositions are administered by intravenous infusion or injection. In certain embodiments, the compositions are administered by subcutaneous injection. In certain embodiments, the compositions are administered by intrathecal injection.

In some embodiments, the administration is via a depot injection. A depot injection may release the RNAi agent in a consistent way over a prolonged time period. Thus, a depot injection may reduce the frequency of dosing needed to obtain a desired effect, e.g., a desired inhibition of ALK, or a therapeutic or prophylactic effect. A depot injection may also provide more consistent serum concentrations. Depot injections may include subcutaneous injections or intramuscular injections. For example, the depot injection may be a subcutaneous injection.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used for intracranial, intravenous, subcutaneous, arterial, or epidural infusions. In one embodiment, the infusion pump is a subcutaneous infusion pump. In other embodiments, the pump is a surgically implanted pump that delivers the RNAi agent to the CNS.

The mode of administration may be chosen based upon whether local or systemic treatment is desired and based upon the area to be treated. The route and site of administration may be chosen to enhance targeting.

In one aspect, the present disclosure also provides methods for inhibiting the expression of an ALK gene in a mammal. The methods include administering to the mammal a composition comprising a dsRNA that targets an ALK gene in a cell of the mammal, thereby inhibiting expression of the ALK gene in the cell. Reduction in gene expression can be assessed by any methods known it the art and by methods, e.g. qRT-PCR, described herein. Reduction in protein production can be assessed by any methods known it the art and by methods, e.g. ELISA, described herein. In one embodiment, a CNS biopsy sample or a cerebrospinal fluid (CSF) sample serves as the tissue material for monitoring the reduction in ALK gene or protein expression (or of a proxy therefore).

The present disclosure further provides methods of treatment of a subject in need thereof. The treatment methods of the disclosure include administering an RNAi agent of the disclosure to a subject, e.g., a subject that would benefit from inhibition of ALK expression, such as a subject in need of losing weight or supporting weight maintenance, in a therapeutically effective amount of an RNAi agent targeting an ALK gene or a pharmaceutical composition comprising an RNAi agent targeting an ALK gene.

In addition, the present disclosure provides methods of preventing, treating or inhibiting the progression of an ALK-associated disease or condition in a subject. The methods include administering to the subject a therapeutically effective amount of any of the RNAi agent, e.g., dsRNA agents, or the pharmaceutical composition provided herein, thereby preventing, treating or inhibiting the progression of an ALK-associated disease or condition in the subject.

An RNAi agent of the disclosure may be administered as a “free RNAi agent.” A free RNAi agent is administered in the absence of a pharmaceutical composition. The naked RNAi agent may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution containing the RNAi agent can be adjusted such that it is suitable for administering to a subject.

Alternatively, an RNAi agent of the disclosure may be administered as a pharmaceutical composition, such as a dsRNA liposomal formulation.

In one embodiment, the subject is a human. Subjects that would benefit from a reduction or inhibition of ALK gene expression include those having an ALK-associated disease, such as type 2 diabetes, obesity, or an obesity-associated disorder, and those in need or desire of losing weight or supporting weight maintenance. Exemplary obesity-associated disorders include, but are not limited to type 2 diabetes, hypertension, dyslipidemia, atherosclerosis, coronary heart disease, stroke, metabolic syndrome, gallbladder disease, fatty liver, osteoarthritis, sleep apnea, breathing problems, various types of cancer (e.g., endometrial cancer, esophageal adenocarcinoma, gastric cardia cancer, liver cancer, kidney cancer, pancreatic cancer), mental illness (e.g., depression, anxiety), body pain, and difficulty with physical functioning. In one aspect, an ALK-associated disease is obesity. Obesity refers to a condition in which an individual weighs more than a healthy reference weight range as a result of excessive accumulation of energy. The additional weight is typically retained in the form of fat under the skin or around the viscera. Obesity is a chronic metabolic disorder and its prevalence continues to increase in many areas of the world. Obesity is the major risk factor for serious diseases such as type 2 diabetes, hypertension, dyslipidemia, atherosclerosis, coronary heart disease, stroke, gallbladder disease, osteoarthritis, sleep apnea, various types of cancer (e.g., endometrial cancer, esophageal adenocarcinoma, gastric cardia cancer, liver cancer, kidney cancer, pancreatic cancer), and mental illness (e.g., depression, anxiety). (See, e.g., World Health Organ. Tech. Rep. Ser. (2000) 894: i-xii, 1-253). In addition, obesity can be associated with body pain, breathing difficulty, difficulty with physical functioning, social stigma, low self-esteem, and poor quality of life generally.

Empirical data suggests that a weight loss of at least 10% of the initial weight results in a considerable decrease in the risk of obesity related co-morbidities (World Health Organ. Tech. Rep. Ser. (2000) 894: i-xii, 1-253). Obesity is induced when the amount of energy intake exceeds the amount of energy consumed. Thus, decreasing the amount of energy intake and increasing the amount of energy consumption by promoting in vivo metabolism would ameliorate obesity and obesity-associated disorders. Once weight loss is accomplished, subjects need to maintain the balance between energy intake and energy consumption to avoid regaining lost weight. Such regression risks reducing or potentially completely reversing any benefits that were associated with the loss of weight.

In one embodiment, an ALK-associated disorder is type 2 diabetes, used interchangeably with type 2 diabetes mellitus or type II diabetes. Type 2 diabetes is a chronic disorder with impaired glucose intolerance due to insulin resistance or relative insulin deficiency. Symptoms and signs of type 2 diabetes include increased thirst, frequent urination, increased hunger, fatigue, blurred vision, slow-healing sores, frequent infections, and areas of darkened skin. Uncontrolled type 2 diabetes leads to serious complications including diabetic retinopathy, diabetic vasculopathy, and diabetic nephropathy. Being overweight or obese is a major modifiable risk factor for type 2 diabetes, and other risk factors include physical inactivity and family history. Early stage type 2 diabetes may be controlled by dietary regimen and weight loss. In advanced stages, type 2 diabetes can be treated by medication (e.g., metformin, sulfonylureas, meglitinides) or insulin injection.

In one embodiment, an obesity-associated disorder is coronary heart disease. Coronary heart disease is a condition in which a coronary artery cannot supply sufficient oxygen to the heart, and is often caused by atherosclerosis and plaque formation within coronary arteries. Symptoms of coronary heart disease include chest pain and chest discomfort. Coronary heart disease includes myocardial infarction (heart attack) and angina, and can cause significant disability or death. Risk factors include type 2 diabetes, hypertension, dyslipidemia, for all of which obesity is a risk factor. Other risk factors include age, smoking, and stress.

In one aspect, an ALK-associated condition is a need or desire to lose weight or maintain weight. The invention provides a non-therapeutic use of an agent of the invention to maintain a healthy body composition, for example after a period of weight loss.

The disclosure further provides methods for the use of an RNAi agent or a pharmaceutical composition thereof, e.g., for treating a subject that would benefit from reduction or inhibition of ALK expression, e.g., a subject having an ALK-associated disorder, in combination with other pharmaceuticals or other therapeutic methods, e.g., with known pharmaceuticals or known therapeutic methods, such as, for example, those which are currently employed for treating these disorders. For example, in certain embodiments, an RNAi agent targeting ALK is administered in combination with, e.g., an agent useful in treating an ALK-associated disorder as described elsewhere herein or as otherwise known in the art. For example, additional agents suitable for treating a subject that would benefit from reduction in ALK expression, e.g., a subject having an ALK-associated disorder, such as type 2 diabetes, obesity, or an obesity-associated disorder, or a subject in need of weight loss or weight maintenance, may include agents currently used to treat or prevent these ALK-associated disorders or treat or prevent symptoms of these ALK-associated disorders. The RNAi agent and additional therapeutic agents or methods may be administered at the same time or in the same combination, e.g., intrathecally. Alternatively, the additional therapeutic agent or method can be administered as part of a separate composition or at separate times or by another method known in the art or described herein, in conjunction with or separately from administration of the RNAi agent of the present invention.

For example, additional therapeutic agents and therapeutic methods suitable for preventing or treating type 2 diabetes, obesity, supporting weight loss, or supporting weight maintenance includes a diet regimen (e.g., a low-calorie diet), an exercise regimen, behavior therapy (e.g., counseling, support group therapy), medications (e.g., orlistat (Alli, Xenical), phentermine and topiramate (Qsymia), bupropion and naltrexone (Contrave), liraglutide (Saxenda, Victoza)), endoscopic procedures to reduce stomach volume or to place a balloon in the stomach, and bariatric surgery (e.g., gastric bypass, sleeve gastrectomy, gastric band, biliopancreatic diversion, duodenal switch). In addition, agents that decrease or otherwise affect the ALK activity may be used in combination with the agent or method of the present invention. Exemplary agents that decrease or otherwise affect the ALK activity include, but are not limited to, Acetylcysteine, Alectinib, Brigatinib, Ceritinib, Crizotinib, Emodin, Everolimus, Foretinib, Heparin, Herbimycin, Hydrocortisone, lapatinib, Masoprocol, NVP-TAE684, PF-04254644, Pyrimidine, Simvastatin, Sucrose octasulfate, Tanespimycin, Tivozanib, Tretinoin, and Vibramycin.

In one embodiment, the method includes administering a composition featured herein such that expression of the target ALK gene is decreased, for at least one month. In certain embodiments, expression is decreased for at least 2 months, 3 months, or 6 months.

Preferably, the RNAi agents useful for the methods and compositions featured herein specifically target RNAs (primary or processed) of the target ALK gene. Compositions and methods for inhibiting the expression of these genes using RNAi agents can be prepared and performed as described herein.

Administration of the dsRNA according to the methods of the disclosure may result in a reduction of the severity, signs, symptoms, or markers of such diseases or disorders in a patient with an ALK-associated disorder. By “reduction” in this context is meant a statistically significant or clinically significant decrease in such level. The reduction can be, for example, at least 5%, 10%, 15%, 20%, 25%, 30%, 35%, 40%, 45%, 50%, 55%, 60%, 65%, 70%, 75%, 80%, 85%, 90%, 95%, or about 100%.

Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. For example, efficacy of treatment of an ALK-associated disorder may be assessed, for example, by periodic monitoring of a subject’s marker’s and/or symptoms. Comparisons of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of an RNAi agent targeting ALK or pharmaceutical composition thereof, “effective against” an ALK-associated disorder indicates that administration in a clinically appropriate manner results in a beneficial effect for at least a statistically significant fraction of patients, such as an improvement of symptoms, a cure, a reduction in disease, extension of life, improvement in quality of life, or other effect generally recognized as positive by medical doctors familiar with treating ALK-associated disorders and the related causes.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given RNAi agent drug or formulation of that drug can also be judged using an experimental animal model for the given disease as known in the art. When using an experimental animal model, efficacy of treatment is evidenced when a statistically significant reduction in a marker or symptom is observed.

Alternatively, the efficacy can be measured by a reduction in the severity of disease as determined by one skilled in the art of diagnosis based on a clinically accepted disease severity grading scale. Any positive change resulting in e.g., lessening of severity of disease measured using the appropriate scale, represents adequate treatment using an RNAi agent or RNAi agent formulation as described herein.

Subjects can be administered a therapeutic amount of dsRNA, such as about 0.01 mg/kg to about 200 mg/kg.

The RNAi agent can be administered intrathecally, via intravitreal injection, or by intravenous infusion over a period of time, on a regular basis. In certain embodiments, after an initial treatment regimen, the treatments can be administered on a less frequent basis. Administration of the RNAi agent can reduce ALK levels, e.g., in a cell, tissue, blood, CSF sample or other compartment of the patient.

Before administration of a full dose of the RNAi agent, patients can be administered a smaller dose, such as a 5% infusion reaction, and monitored for adverse effects, such as an allergic reaction. In another example, the patient can be monitored for unwanted immunostimulatory effects, such as increased cytokine (e.g., TNF-alpha or INF-alpha) levels.

Alternatively, the RNAi agent can be administered subcutaneously, i.e., by subcutaneous injection. One or more injections may be used to deliver the desired, e.g., monthly dose of RNAi agent to a subject. The injections may be repeated over a period of time. The administration may be repeated on a regular basis. In certain embodiments, after an initial treatment regimen, the treatments can be administered on a less frequent basis. A repeat-dose regimen may include administration of a therapeutic amount of RNAi agent on a regular basis, such as monthly or extending to once a quarter, twice per year, once per year. In certain embodiments, the RNAi agent is administered about once per month to about once per quarter (i.e., about once every three months).

Representative publications that teach agents capable of decreasing the activity of ALK, including nucleic acid products that interfere with the ALK gene expression or inhibit its expression, and methods or therapeutic use thereof include, but are not limited to, PCT publication WO 2019/101490, the entire contents of which are hereby incorporated herein by reference.

Unless otherwise defined, all technical and scientific terms used herein have the same meaning as commonly understood by one of ordinary skill in the art to which this invention belongs. Although methods and materials similar or equivalent to those described herein can be used in the practice or testing of the RNAi agents and methods featured in the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

An informal Sequence Listing is filed herewith and forms part of the specification as filed.

### EXAMPLES

### Example 1. iRNA Synthesis

### Source of Reagents

Where the source of a reagent is not specifically given herein, such reagent can be obtained from any supplier of reagents for molecular biology at a quality/purity standard for application in molecular biology.

### siRNA Design

siRNAs targeting the human ALK gene (human: NCBI refseqID NM_004304.4; NCBI GeneID: 238) were designed using custom R and Python scripts. The human NM_004304.4 REFSEQ mRNA has a length of 6267 bases.

A detailed list of the unmodified ALK sense and antisense strand nucleotide sequences is shown in Table 2. A detailed list of the modified ALK sense and antisense strand nucleotide sequences is shown in Tables 3 and 4.

It is to be understood that, throughout the application, a duplex name without a decimal is equivalent to a duplex name with a decimal which merely references the batch number of the duplex. For example, AD-1290270 is equivalent to AD-1290270.1.

### siRNA Synthesis

siRNAs were synthesized and annealed using routine methods known in the art.

Briefly, siRNA sequences were synthesized at 1 µmol scale on a Mermade 192 synthesizer (BioAutomation) using the solid support mediated phosphoramidite chemistry. The solid support was controlled pore glass (500 A) loaded with custom GalNAc ligand or universal solid support (AM biochemical). Ancillary synthesis reagents, 2′-F and 2′-O-Methyl RNA and deoxy phosphoramidites were obtained from Thermo-Fisher (Milwaukee, WI) and Hongene (China). 2′F 2′-O-Methyl, GNA (glycol nucleic acids), 5′phosphate and other modifications were introduced using the corresponding phosphoramidites. Synthesis of 3′ GalNAc conjugated single strands was performed on a GalNAc modified CPG support. Custom CPG universal solid support was used for the synthesis of antisense single strands. Coupling time for all phosphoramidites (100 mM in acetonitrile) was 5 min employing 5-Ethylthio-1H-tetrazole (ETT) as activator (0.6 M in acetonitrile). Phosphorothioate linkages were generated using a 50 mM solution of 3-((Dimethylamino-methylidene) amino)-3H-1,2,4-dithiazole-3-thione (DDTT, obtained from Chemgenes (Wilmington, MA, USA)) in anhydrous acetonitrile/pyridine (1:1 v/v). Oxidation time was 3 minutes. All sequences were synthesized with final removal of the DMT group (“DMT off”).

Upon completion of the solid phase synthesis, oligoribonucleotides were cleaved from the solid support and deprotected in sealed 96 deep well plates using 200 µL Aqueous Methylamine reagents at 60° C. for 20 minutes. For sequences containing 2′ ribo residues (2′-OH) that are protected with a tert-butyl dimethyl silyl (TBDMS) group, a second step deprotection was performed using TEA.3HF (triethylamine trihydro fluoride) reagent. To the methylamine deprotection solution, 200µL of dimethyl sulfoxide (DMSO) and 300 µl TEA.3HF reagent was added and the solution was incubated for additional 20 min at 60° C. At the end of cleavage and deprotection step, the synthesis plate was allowed to come to room temperature and was precipitated by addition of 1 mL of acetontile: ethanol mixture (9:1). The plates were cooled at -80° C. for 2 hrs, supernatant decanted carefully with the aid of a multi-channel pipette. The oligonucleotide pellet was re-suspended in 20 mM NaOAc buffer and were desalted using a 5 mL HiTrap size exclusion column (GE Healthcare) on an AKTA Purifier System equipped with an A905 autosampler and a Frac 950 fraction collector. Desalted samples were collected in 96-well plates. Samples from each sequence were analyzed by LC-MS to confirm the identity, UV (260 nm) for quantification and a selected set of samples by IEX chromatography to determine purity.

Annealing of single strands was performed on a Tecan liquid handling robot. Equimolar mixture of sense and antisense single strands were combined and annealed in 96 well plates. After combining the complementary single strands, the 96-well plate was sealed tightly and heated in an oven at 100° C. for 10 minutes and allowed to come slowly to room temperature over a period 2-3 hours. The concentration of each duplex was normalized to 10 µM in 1X PBS and then submitted for in vitro screening assays.

### Example 2. In Vitro Screening Methods

### Cell Culture and 384-Well Transfections

Cos-7 (ATCC) are transfected by adding 5µl of 1 ng/µL, diluted in Opti-MEM, C9orf72 intron 1 psiCHECK2 vector (Blue Heron Biotechnology), 4.9 µL of Opti-MEM plus 0.1 µL of Lipofectamine 2000 per well (Invitrogen, Carlsbad CA. cat #11668-019) to 5 µL of siRNA duplexes per well, with 4 replicates of each siRNA duplex, into a 384-well plate, and incubated at room temperature for 15 minutes. Thirty-five µL of Dulbecco’s Modified Eagle Medium (ThermoFisher) containing ~5 ×10³ cells are then added to the siRNA mixture. Cells are incubated for 48 hours followed by Firefly (transfection control) and Renilla (fused to target sequence) luciferase measurements. Three dose experiments are performed at 10 nM, 1 nM, and 0.1 nM.

BE-C (ATCC) are transfected by adding 4.9 µL of Opti-MEM plus 0.1µl of RNAiMAX per well (Invitrogen, Carlsbad CA. cat # 13778-150) to 5 µL of siRNA duplexes per well, with 4 replicates of each siRNA duplex, into a 384-well plate, and incubated at room temperature for 15 minutes. Forty µL of 1:1 mixture of Minimum Essential Medium and F12 Medium (ThermoFisher) containing ~5 ×10³ cells are then added to the siRNA mixture. Cells are incubated for 48 hours prior to RNA purification. Three dose experiments are performed at 10 nM, 1 nM, and 0.1 nM.

Neuro-2a (ATCC) are transfected by adding 4.9 µL of Opti-MEM plus 0.1 µL of RNAiMAX per well (Invitrogen, Carlsbad CA. cat # 13778-150) to 5 µL of siRNA duplexes per well, with 4 replicates of each siRNA duplex, into a 384-well plate, and incubated at room temperature for 15 minutes. Forty µL of Minimum Essential Medium (ThermoFisher) containing ~5 ×10³ cells are then added to the siRNA mixture. Cells are incubated for 48 hours prior to RNA purification. Three dose experiments are performed at 10 nM, 1 nM, and 0.1 nM.

### Total RNA Isolation Using DYNABEADS mRNA Isolation Kit (Invitrogen™, Part #: 610-12)

Cells are lysed in 75 µL of Lysis/Binding Buffer containing 3 µL of beads per well and are mixed for 10 minutes on an electrostatic shaker. The washing steps are automated on a Biotek EL406, using a magnetic plate support. Beads are washed (in 90 µL) once in Buffer A, once in Buffer B, and twice in Buffer E, with aspiration steps in between. Following a final aspiration, complete 10 µL RT mixture is added to each well, as described below.

### cDNA Synthesis Using ABI High Capacity cDNA Reverse Transcription Kit (Applied Biosvstems, Foster City, CA, Cat #4368813)

A master mix of 1 µl 10X Buffer, 0.4 µL 25X dNTPs, 1 µL Random primers, 0.5 µL Reverse Transcriptase, 0.5 µL RNase inhibitor and 6.6 µL of H₂O per reaction is added per well. Plates are sealed, are agitated for 10 minutes on an electrostatic shaker, and then are incubated at 37° C. for 2 hours. Following this, the plates are agitated at 80° C. for 8 minutes.

### Real Time PCR

Two microliter (µL) of cDNA is added to a master mix containing 0.5 µL of human GAPDH TaqMan Probe and 0.5 µL human ALK probe, or 0.5 µL mouse GAPDH TaqMan Probe and 0.5 µL mouse ALK probe, or 0.5 µL cynomolgus monkey GAPDH TaqMan Probe and 0.5 µL cynomolgus monkey ALK probe, 2 µL nuclease-free water and 5 µL Lightcycler 480 probe master mix (Roche Cat # 04887301001) per well in a 384 well plates (Roche cat # 04887301001). Real time PCR is done in a LightCycler480 Real Time PCR system (Roche). Each duplex is tested at least two times and data are normalized to cells transfected with a non-targeting control siRNA. To calculate relative fold change, real time data are analyzed using the ΔΔCt method and are normalized to assays performed with cells transfected with a non-targeting control siRNA.

### Example 3. Single Dose Reporter Screen for Human ALK siRNAs

Hepal-6, mouse hepatoma cells, were transfected by adding 5 µL of 1 ng/µL of each siRNA duplex diluted in Opti-MEM, 75 ng V127 human ALK reporter construct, plus 0.5 µl of Lipofectamine 2000 to an individual well in a 96-well plate, with 4 replicates of each siRNA duplex. The mixture was incubated at room temperature for 15 minutes. Eighty µL of complete growth media without antibiotic containing ~2 ×10⁴ Hepal-6 cells was then added to the siRNA mixture. Cells were incubated for 24 hours followed by Firefly (transfection control) and Renilla (fused to target sequence) luciferase measurements. A single dose experiment was performed at 10 nM final duplex concentration. The results are shown in Table 5 and are presented as the average percent ALK mRNA remaining as compared to a negative control.

Table 1. Abbreviations of nucleotide monomers used in nucleic acid sequence representation. It will be understood that these monomers, when present in an oligonucleotide, are mutually linked by 5′-3′-phosphodiester bonds; and it is understood that when the nucleotide contains a 2′-fluoro modification, then the fluoro replaces the hydroxy at that position in the parent nucleotide (i.e., it is a 2′-deoxy-2′-fluoronucleotide).

| | |
|---|---|
| Abbreviation | Nucleotide(s) |
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide, modified or unmodified |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amidodecanoyl)]-4-hydroxyprolinol (Hyp-(GalNAc-alkyl)3) |
| Y34 | 2-hydroxymethyl-tetrahydrofurane-4-methoxy-3-phosphate (abasic 2′-OMe furanose) |
| Y44 | inverted abasic DNA (2-hydroxymethyl-tetrahydrofurane-5-phosphate) |
| (Agn) | Adenosine-glycol nucleic acid (GNA) |
| (Cgn) | Cytidine-glycol nucleic acid (GNA) |
| (Ggn) | Guanosine-glycol nucleic acid (GNA) |
| (Tgn) | Thymidine-glycol nucleic acid (GNA) S-Isomer |
| P | Phosphate |
| VP | Vinyl-phosphonate |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3′-phosphorothioate |

TABLE 2

Unmodified Sense and Antisense Strand Sequences of Human ALK dsRNA Agents

| Duplex Name | Sense Sequence 5′ to 3′ | SEQ ID NO: | Range in NM_00430 4.4 | Antisense Sequence 5′ to 3′ | SEQ ID NO | Range in NM_004304. 4 |
|---|---|---|---|---|---|---|
| AD-1289971 | GCAGAUGCGAU CCAGCGGCUA | 14 | 24-44 | UAGCCGCUGGAUC GCAUCUGCCU | 314 | 22-44 |
| AD-1289972 | CGGUGGUAGCA GCUGGUACCA | 15 | 57-77 | UGGUACCAGCUGC UACCACCGCU | 315 | 55-77 |
| AD-1289973 | GCGCUGAUGAU GGGUGAGGAA | 16 | 157-177 | UUCCUCACCCAUC AUCAGCGCCC | 316 | 155-177 |
| AD-1289974 | UGCCUGCGAAC UCUGAGGAGA | 17 | 245-265 | UCUCCUCAGAGUU CGCAGGCACU | 317 | 243-265 |
| AD-1289975 | GGACGCUGCAA ACUUGCGCAA | 18 | 286-306 | UUGCGCAAGUUU GCAGCGUCCUU | 318 | 284-306 |
| AD-1289976 | GCUGGGAUUCA CGCCCAGAAA | 19 | 314-334 | UUUCUGGGCGUG AAUCCCAGCCC | 319 | 312-334 |
| AD-1289977 | GCCCAGAAGUU CAGCAGGCAA | 20 | 326-346 | UUGCCUGCUGAAC UUCUGGGCGU | 320 | 324-346 |
| AD-1289978 | GCAGACAGUCC GAAGCCUUCA | 21 | 343-363 | UGAAGGCUUCGG ACUGUCUGCCU | 321 | 341-363 |
| AD-1289979 | CAGCGGAGAGA UAGCUUGAGA | 22 | 366-386 | UCUCAAGCUAUCU CUCCGCUGCG | 322 | 364-386 |
| AD-1289980 | UGAGGGUGCGC AAGACGGCAA | 23 | 382-402 | UUGCCGUCUUGCG CACCCUCAAG | 323 | 380-402 |
| AD-1289981 | GGGCAGAAGAG CUUGGAGGAA | 24 | 430-450 | UUCCUCCAAGCUC UUCUGCCCGG | 324 | 428-450 |
| AD-1289982 | GAGCCAAAAGG AACGCAAAAA | 25 | 448-468 | UUUUUGCGUUCC UUUUGGCUCCU | 325 | 446-468 |
| AD-1289983 | AAAGGCGGCCA GGACAGCGUA | 26 | 465-485 | UACGCUGUCCUGG CCGCCUUUUG | 326 | 463-485 |
| AD-1289984 | CCGCCGUUCUC AGCCUUAAAA | 27 | 499-519 | UUUUAAGGCUGA GAACGGCGGCU | 327 | 497-519 |
| AD-1289985 | CCUUAAAAGUU GCAGAGAUUA | 28 | 512-532 | UAAUCUCUGCAAC UUUUAAGGCU | 328 | 510-532 |
| AD-1289986 | GACGGUACCCA ACUGCCACCA | 29 | 587-607 | UGGUGGCAGUUG GGUACCGUCCU | 329 | 585-607 |
| AD-1289987 | CUGCCACCUCCC UUCAACCAA | 30 | 599-619 | UUGGUUGAAGGG AGGUGGCAGUU | 330 | 597-619 |
| AD-1289988 | UCAACCAUAGU AGUUCCUCUA | 31 | 612-632 | UAGAGGAACUAC UAUGGUUGAAG | 331 | 610-632 |
| AD-1289989 | GUUCCUCUGUA CCGAGCGCAA | 32 | 624-644 | UUGCGCUCGGUAC AGAGGAACUA | 332 | 622-644 |
| AD-1289990 | CGAGCGCAGCG AGCUACAGAA | 33 | 636-656 | UUCUGUAGCUCGC UGCGCUCGGU | 333 | 634-656 |
| AD-1289991 | GGCUCAAGGUC CCAGCCAGUA | 34 | 688-708 | UACUGGCUGGGA CCUUGAGCCUC | 334 | 686-708 |
| AD-1289992 | GCCAGUGAGCC CAGUGUGCUA | 35 | 702-722 | UAGCACACUGGGC UCACUGGCUG | 335 | 700-722 |
| AD-1289993 | AGUGUGCUUGA GUGUCUCUGA | 36 | 714-734 | UCAGAGACACUCA AGCACACUGG | 336 | 712-734 |
| AD-1289994 | GGUCUGUUUCA UUUAGACUCA | 37 | 754-774 | UGAGUCUAAAUG AAACAGACCUG | 337 | 752-774 |
| AD-1289995 | CUGCUCGCCUCC GUGCAGUUA | 38 | 774-794 | UAACUGCACGGA GGCGAGCAGGA | 338 | 772-794 |
| AD-1289996 | GAAAGCAAGAG ACUUGCGCGA | 39 | 798-818 | UCGCGCAAGUCUC UUGCUUUCCC | 339 | 796-818 |
| AD-1289997 | GCGCGCACGCA CAGUCCUCUA | 40 | 813-833 | UAGAGGACUGUG CGUGCGCGCAA | 340 | 811-833 |
| AD-1289998 | UCCUCUGGAGA UCAGGUGGAA | 41 | 827-847 | UUCCACCUGAUCU CCAGAGGACU | 341 | 825-847 |
| AD-1289999 | AGGAGCCGCUG GGUACCAAGA | 42 | 847-867 | UCUUGGUACCCAG CGGCUCCUUC | 342 | 845-867 |
| AD-1290000 | GUACCAAGGAC UGUUCAGAGA | 43 | 859-879 | UCUCUGAACAGUC CUUGGUACCC | 343 | 857-879 |
| AD-1290001 | UCAGAGCCUCU UCCCAUCUCA | 44 | 873-893 | UGAGAUGGGAAG AGGCUCUGAAC | 344 | 871-893 |
| AD-1290002 | CCGGAGAGCAG UGUAAACGGA | 45 | 919-939 | UCCGUUUACACUG CUCUCCGGGC | 345 | 917-939 |
| AD-1290003 | GGGAGCCAUCG GGCUCCUGUA | 46 | 955-975 | UACAGGAGCCCGA UGGCUCCCAU | 346 | 953-975 |
| AD-1290004 | UGCUGCUUUCC ACGGCAGCUA | 47 | 987-1007 | UAGCUGCCGUGG AAAGCAGCAGC | 347 | 985-1007 |
| AD-1290005 | CCACUCAGCUA CUCGCGCCUA | 48 | 1079-1099 | UAGGCGCGAGUA GCUGAGUGGCU | 348 | 1077-1099 |
| AD-1290006 | CAGAGGAAGAG UCUGGCAGUA | 49 | 1100-1120 | UACUGCCAGACUC UUCCUCUGCA | 349 | 1098-1120 |
| AD-1290007 | CUGGCAGUUGA CUUCGUGGUA | 50 | 1112-1132 | UACCACGAAGUCA ACUGCCAGAC | 350 | 1110-1132 |
| AD-1290008 | UUCGUGGUGCC CUCGCUCUUA | 51 | 1124-1144 | UAAGAGCGAGGG CACCACGAAGU | 351 | 1122-1144 |
| AD-1290009 | UCGCUCUUCCG UGUCUACGCA | 52 | 1136-1156 | UGCGUAGACACG GAAGAGCGAGG | 352 | 1134-1156 |
| AD-1290010 | GUCUACGCCCG GGACCUACUA | 53 | 1148-1168 | UAGUAGGUCCCG GGCGUAGACAC | 353 | 1146-1168 |
| AD-1290011 | GACCUACUGCU GCCACCAUCA | 54 | 1160-1180 | UGAUGGUGGCAG CAGUAGGUCCC | 354 | 1158-1180 |
| AD-1290012 | CUCGGAGCUGA AGGCUGGCAA | 55 | 1183-1203 | UUGCCAGCCUUCA GCUCCGAGGA | 355 | 1181-1203 |
| AD-1290013 | GCUGUCCAGGG UGCUGAAGGA | 56 | 1318-1338 | UCCUUCAGCACCC UGGACAGCGU | 356 | 1316-1338 |
| AD-1290014 | CGUGCCAAGCA GUUGGUGCUA | 57 | 1361-1381 | UAGCACCAACUGC UUGGCACGCC | 357 | 1359-1381 |
| AD-1290015 | UUGGUGCUGGA GCUGGGCGAA | 58 | 1373-1393 | UUCGCCCAGCUCC AGCACCAACU | 358 | 1371-1393 |
| AD-1290016 | GAGGCGAUCUU GGAGGGUUGA | 59 | 1394-1414 | UCAACCCUCCAAG AUCGCCUCCU | 359 | 1392-1414 |
| AD-1290017 | CUGCUCCAGUU CAAUCUCAGA | 60 | 1445-1465 | UCUGAGAUUGAA CUGGAGCAGCC | 360 | 1443-1465 |
| AD-1290018 | CAGCGAGCUGU UCAGUUGGUA | 61 | 1462-1482 | UACCAACUGAACA GCUCGCUGAG | 361 | 1460-1482 |
| AD-1290019 | UUGGUGGAUUC GCCAAGGCGA | 62 | 1477-1497 | UCGCCUUGGCGAA UCCACCAACU | 362 | 1475-1497 |
| AD-1290020 | GGGCGACUGAG GAUCCGCCUA | 63 | 1499-1519 | UAGGCGGAUCCUC AGUCGCCCUU | 363 | 1497-1519 |
| AD-1290021 | AUCCGCCUGAU GCCCGAGAAA | 64 | 1511-1531 | UUUCUCGGGCAUC AGGCGGAUCC | 364 | 1509-1531 |
| AD-1290022 | CCGAGAAGAAG GCGUCGGAAA | 65 | 1524-1544 | UUUCCGACGCCUU CUUCUCGGGC | 365 | 1522-1544 |
| AD-1290023 | GGGCAGAGAGG GAAGGCUGUA | 66 | 1546-1566 | UACAGCCUUCCCU CUCUGCCCAC | 366 | 1544-1566 |
| AD-1290024 | GGCUGUCCGCG GCAAUUCGCA | 67 | 1560-1580 | UGCGAAUUGCCGC GGACAGCCUU | 367 | 1558-1580 |
| AD-1290025 | CCUUCUCUUCC AGAUCUUCGA | 68 | 1594-1614 | UCGAAGAUCUGG AAGAGAAGGCG | 368 | 1592-1614 |
| AD-1290026 | ACUGGUCAUAG CUCCUUGGAA | 69 | 1616-1636 | UUCCAAGGAGCU AUGACCAGUCC | 369 | 1614-1636 |
| AD-1290027 | CCUUGGAAUCA CCAACAAACA | 70 | 1629-1649 | UGUUUGUUGGUG AUUCCAAGGAG | 370 | 1627-1649 |
| AD-1290028 | CAAACAUGCCU UCUCCUUCUA | 71 | 1644-1664 | UAGAAGGAGAAG GCAUGUUUGUU | 371 | 1642-1664 |
| AD-1290029 | CUUCUCCUGAU UAUUUUACAA | 72 | 1659-1679 | UUGUAAAAUAAU CAGGAGAAGGA | 372 | 1657-1679 |
| AD-1290030 | UUUUACAUGGA AUCUCACCUA | 73 | 1672-1692 | UAGGUGAGAUUC CAUGUAAAAUA | 373 | 1670-1692 |
| AD-1290031 | UCUCACCUGGA UAAUGAAAGA | 74 | 1684-1704 | UCUUUCAUUAUCC AGGUGAGAUU | 374 | 1682-1704 |
| AD-1290032 | AUGAAAGACUC CUUCCCUUUA | 75 | 1697-1717 | UAAAGGGAAGGA GUCUUUCAUUA | 375 | 1695-1717 |
| AD-1290033 | UUCCCUUUCCU GUCUCAUCGA | 76 | 1709-1729 | UCGAUGAGACAG GAAAGGGAAGG | 376 | 1707-1729 |
| AD-1290034 | CAUCGCAGCCG AUAUGGUCUA | 77 | 1724-1744 | UAGACCAUAUCG GCUGCGAUGAG | 377 | 1722-1744 |
| AD-1290035 | GGAGUGCAGCU UUGACUUCCA | 78 | 1744-1764 | UGGAAGUCAAAG CUGCACUCCAG | 378 | 1742-1764 |
| AD-1290036 | CCACUGCAUGA CCUCAGGAAA | 79 | 1787-1807 | UUUCCUGAGGUC AUGCAGUGGAG | 379 | 1785-1807 |
| AD-1290037 | CAGGAACCAGA GCUGGUCCUA | 80 | 1801-1821 | UAGGACCAGCUCU GGUUCCUGAG | 380 | 1799-1821 |
| AD-1290038 | UCCCAGAUGGA CUUGCUGGAA | 81 | 1847-1867 | UUCCAGCAAGUCC AUCUGGGAGG | 381 | 1845-1867 |
| AD-1290039 | AGAGCGUUCUA AGGAGAUGCA | 82 | 1879-1899 | UGCAUCUCCUUAG AACGCUCUGC | 382 | 1877-1899 |
| AD-1290040 | AUGCCCAGAGG CUCCUUUCUA | 83 | 1895-1915 | UAGAAAGGAGCC UCUGGGCAUCU | 383 | 1893-1915 |
| AD-1290041 | UCCUUUCUCCU UCUCAACACA | 84 | 1907-1927 | UGUGUUGAGAAG GAGAAAGGAGC | 384 | 1905-1927 |
| AD-1290042 | AGCUGACUCCA AGCACACCAA | 85 | 1930-1950 | UUGGUGUGCUUG GAGUCAGCUGA | 385 | 1928-1950 |
| AD-1290043 | GCACACCAUCC UGAGUCCGUA | 86 | 1942-1962 | UACGGACUCAGG AUGGUGUGCUU | 386 | 1940-1962 |
| AD-1290044 | GAGUCCGUGGA UGAGGAGCAA | 87 | 1954-1974 | UUGCUCCUCAUCC ACGGACUCAG | 387 | 1952-1974 |
| AD-1290045 | CAGCAGUGAGC ACUGCACACA | 88 | 1972-1992 | UGUGUGCAGUGC UCACUGCUGCU | 388 | 1970-1992 |
| AD-1290046 | UGCACACUGGC CGUCUCGGUA | 89 | 1985-2005 | UACCGAGACGGCC AGUGUGCAGU | 389 | 1983-2005 |
| AD-1290047 | UCGGUGCACAG GCACCUGCAA | 90 | 2000-2020 | UUGCAGGUGCCU GUGCACCGAGA | 390 | 1998-2020 |
| AD-1290048 | CUCUGGAAGGU ACAUUGCCCA | 91 | 2023-2043 | UGGGCAAUGUAC CUUCCAGAGGG | 391 | 2021-2043 |
| AD-1290049 | GCUGCAAGAGA GAUCCUCCUA | 92 | 2063-2083 | UAGGAGGAUCUC UCUUGCAGCCU | 392 | 2061-2083 |
| AD-1290050 | AUCCUCCUGAU GCCCACUCCA | 93 | 2075-2095 | UGGAGUGGGCAU CAGGAGGAUCU | 393 | 2073-2095 |
| AD-1290051 | CAGGGAAGCAU GGUUGGACAA | 94 | 2094-2114 | UUGUCCAACCAUG CUUCCCUGGA | 394 | 2092-2114 |
| AD-1290052 | UUGGACAGUGC UCCAGGGAAA | 95 | 2107-2127 | UUUCCCUGGAGCA CUGUCCAACC | 395 | 2105-2127 |
| AD-1290053 | CCAGGGAAGAA UCGGGCGUCA | 96 | 2119-2139 | UGACGCCCGAUUC UUCCCUGGAG | 396 | 2117-2139 |
| AD-1290054 | CCAGACAACCC AUUUCGAGUA | 97 | 2138-2158 | UACUCGAAAUGG GUUGUCUGGAC | 397 | 2136-2158 |
| AD-1290055 | UCGAGUGGCCC UGGAAUACAA | 98 | 2152-2172 | UUGUAUUCCAGG GCCACUCGAAA | 398 | 2150-2172 |
| AD-1290056 | GGAAUACAUCU CCAGUGGAAA | 99 | 2164-2184 | UUUCCACUGGAG AUGUAUUCCAG | 399 | 2162-2184 |
| AD-1290057 | CAGUGGAAACC GCAGCUUGUA | 100 | 2176-2196 | UACAAGCUGCGG UUUCCACUGGA | 400 | 2174-2196 |
| AD-1290058 | CUGCAGUGGAC UUCUUUGCCA | 101 | 2196-2216 | UGGCAAAGAAGU CCACUGCAGAC | 401 | 2194-2216 |
| AD-1290059 | CCUGAAGAACU GCAGUGAAGA | 102 | 2215-2235 | UCUUCACUGCAGU UCUUCAGGGC | 402 | 2213-2235 |
| AD-1290060 | GGCUCCAAGAU GGCCCUGCAA | 103 | 2246-2266 | UUGCAGGGCCAUC UUGGAGCCUG | 403 | 2244-2266 |
| AD-1290061 | CUCCUUCACUU GUUGGAAUGA | 104 | 2269-2289 | UCAUUCCAACAAG UGAAGGAGCU | 404 | 2267-2289 |
| AD-1290062 | ACAGUCCUCCA GCUUGGGCAA | 105 | 2291-2311 | UUGCCCAAGCUGG AGGACUGUCC | 405 | 2289-2311 |
| AD-1290063 | CUGUGACUUCC ACCAGGACUA | 106 | 2314-2334 | UAGUCCUGGUGG AAGUCACAGGC | 406 | 2312-2334 |
| AD-1290064 | CAGGACUGUGC CCAGGGAGAA | 107 | 2327-2347 | UUCUCCCUGGGCA CAGUCCUGGU | 407 | 2325-2347 |
| AD-1290065 | CAGGGAGAAGA UGAGAGCCAA | 108 | 2339-2359 | UUGGCUCUCAUCU UCUCCCUGGG | 408 | 2337-2359 |
| AD-1290066 | AGCCAGAUGUG CCGGAAACUA | 109 | 2354-2374 | UAGUUUCCGGCAC AUCUGGCUCU | 409 | 2352-2374 |
| AD-1290067 | CGGAAACUGCC UGUGGGUUUA | 110 | 2366-2386 | UAAACCCACAGGC AGUUUCCGGC | 410 | 2364-2386 |
| AD-1290068 | UGCAACUUUGA AGAUGGCUUA | 111 | 2390-2410 | UAAGCCAUCUUCA AAGUUGCAGU | 411 | 2388-2410 |
| AD-1290069 | GAUGGCUUCUG UGGCUGGACA | 112 | 2402-2422 | UGUCCAGCCACAG AAGCCAUCUU | 412 | 2400-2422 |
| AD-1290070 | ACCCAAGGCAC ACUGUCACCA | 113 | 2420-2440 | UGGUGACAGUGU GCCUUGGGUCC | 413 | 2418-2440 |
| AD-1290071 | ACUCCUCAAUG GCAGGUCAGA | 114 | 2444-2464 | UCUGACCUGCCAU UGAGGAGUGU | 414 | 2442-2464 |
| AD-1290072 | GACCCUAAAGG AUGCCCGGUA | 115 | 2464-2484 | UACCGGGCAUCCU UUAGGGUCCU | 415 | 2462-2484 |
| AD-1290073 | GCCCGGUUCCA GGACCACCAA | 116 | 2477-2497 | UUGGUGGUCCUG GAACCGGGCAU | 416 | 2475-2497 |
| AD-1290074 | GACCACCAAGA CCAUGCUCUA | 117 | 2489-2509 | UAGAGCAUGGUC UUGGUGGUCCU | 417 | 2487-2509 |
| AD-1290075 | UGCUCUAUUGC UCAGUACCAA | 118 | 2503-2523 | UUGGUACUGAGC AAUAGAGCAUG | 418 | 2501-2523 |
| AD-1290076 | GCUUCUGAAAG UGCUACAGUA | 119 | 2534-2554 | UACUGUAGCACU UUCAGAAGCGG | 419 | 2532-2554 |
| AD-1290077 | CCAGUGCUACG UUUCCUGCAA | 120 | 2556-2576 | UUGCAGGAAACG UAGCACUGGUC | 420 | 2554-2576 |
| AD-1290078 | ACCGAUCAAGA GCUCUCCAUA | 121 | 2575-2595 | UAUGGAGAGCUC UUGAUCGGUGC | 421 | 2573-2595 |
| AD-1290079 | CUCUCCAUGUG AGCUCCGAAA | 122 | 2587-2607 | UUUCGGAGCUCAC AUGGAGAGCU | 422 | 2585-2607 |
| AD-1290080 | GCUCCGAAUGU CCUGGCUCAA | 123 | 2599-2619 | UUGAGCCAGGAC AUUCGGAGCUC | 423 | 2597-2619 |
| AD-1290081 | UGGCUCAUUCG UGGAGUCUUA | 124 | 2612-2632 | UAAGACUCCACGA AUGAGCCAGG | 424 | 2610-2632 |
| AD-1290082 | GAAACGUGUCC UUGGUGCUAA | 125 | 2637-2657 | UUAGCACCAAGG ACACGUUUCCC | 425 | 2635-2657 |
| AD-1290083 | GUGCUAGUGGA GAACAAAACA | 126 | 2651-2671 | UGUUUUGUUCUC CACUAGCACCA | 426 | 2649-2671 |
| AD-1290084 | GGGAAGGAGCA AGGCAGGAUA | 127 | 2672-2692 | UAUCCUGCCUUGC UCCUUCCCGG | 427 | 2670-2692 |
| AD-1290085 | GGCAGGAUGGU CUGGCAUGUA | 128 | 2684-2704 | UACAUGCCAGACC AUCCUGCCUU | 428 | 2682-2704 |
| AD-1290086 | CGCCUAUGAAG GCUUGAGCCA | 129 | 2707-2727 | UGGCUCAAGCCUU CAUAGGCGGC | 429 | 2705-2727 |
| AD-1290087 | UUGAGCCUGUG GCAGUGGAUA | 130 | 2720-2740 | UAUCCACUGCCAC AGGCUCAAGC | 430 | 2718-2740 |
| AD-1290088 | CAGUGGAUGGU GUUGCCUCUA | 131 | 2732-2752 | UAGAGGCAACACC AUCCACUGCC | 431 | 2730-2752 |
| AD-1290089 | CUCGAUGUGUC UGACAGGUUA | 132 | 2753-2773 | UAACCUGUCAGAC ACAUCGAGGA | 432 | 2751-2773 |
| AD-1290090 | CUGGCUGCAGA UGGUCGCAUA | 133 | 2773-2793 | UAUGCGACCAUCU GCAGCCAGAA | 433 | 2771-2793 |
| AD-1290091 | AUCCAGAGCCA UCGUGGCUUA | 134 | 2806-2826 | UAAGCCACGAUG GCUCUGGAUCC | 434 | 2804-2826 |
| AD-1290092 | GUGGCUUUUGA CAAUAUCUCA | 135 | 2819-2839 | UGAGAUAUUGUC AAAAGCCACGA | 435 | 2817-2839 |
| AD-1290093 | AAUAUCUCCAU CAGCCUGGAA | 136 | 2831-2851 | UUCCAGGCUGAU GGAGAUAUUGU | 436 | 2829-2851 |
| AD-1290094 | AGCCUGGACUG CUACCUCACA | 137 | 2843-2863 | UGUGAGGUAGCA GUCCAGGCUGA | 437 | 2841-2863 |
| AD-1290095 | UACCUCACCAU UAGCGGAGAA | 138 | 2855-2875 | UUCUCCGCUAAUG GUGAGGUAGC | 438 | 2853-2875 |
| AD-1290096 | GGACAAGAUCC UGCAGAAUAA | 139 | 2875-2895 | UUAUUCUGCAGG AUCUUGUCCUC | 439 | 2873-2895 |
| AD-1290097 | AGAAUACAGCA CCCAAAUCAA | 140 | 2889-2909 | UUGAUUUGGGUG CUGUAUUCUGC | 440 | 2887-2909 |
| AD-1290098 | CCAAAUCAAGA AACCUGUUUA | 141 | 2901-2921 | UAAACAGGUUUC UUGAUUUGGGU | 441 | 2899-2921 |
| AD-1290099 | CCUGUUUGAGA GAAACCCAAA | 142 | 2914-2934 | UUUGGGUUUCUC UCAAACAGGUU | 442 | 2912-2934 |
| AD-1290100 | CCCAAACAAGG AGCUGAAACA | 143 | 2929-2949 | UGUUUCAGCUCCU UGUUUGGGUU | 443 | 2927-2949 |
| AD-1290101 | GAAAAUUCACC AAGACAGACA | 144 | 2954-2974 | UGUCUGUCUUGG UGAAUUUUCCC | 444 | 2952-2974 |
| AD-1290102 | CUUUGACCCUA CAGUUCAUUA | 145 | 2980-3000 | UAAUGAACUGUA GGGUCAAAGAU | 445 | 2978-3000 |
| AD-1290103 | AGUUCAUUGGC UGUUCACCAA | 146 | 2992-3012 | UUGGUGAACAGC CAAUGAACUGU | 446 | 2990-3012 |
| AD-1290104 | GCACAGUGCAA CAACGCCUAA | 147 | 3047-3067 | UUAGGCGUUGUU GCACUGUGCCU | 447 | 3045-3067 |
| AD-1290105 | CUACCAGAACU CCAACCUGAA | 148 | 3064-3084 | UUCAGGUUGGAG UUCUGGUAGGC | 448 | 3062-3084 |
| AD-1290106 | CAACCUGAGCG UGGAGGUGGA | 149 | 3076-3096 | UCCACCUCCACGC UCAGGUUGGA | 449 | 3074-3096 |
| AD-1290107 | CAUCCAGAUCU GGAAGGUGCA | 150 | 3118-3138 | UGCACCUUCCAGA UCUGGAUGCC | 450 | 3116-3138 |
| AD-1290108 | GAAGGUGCCAG CCACCGACAA | 151 | 3130-3150 | UUGUCGGUGGCU GGCACCUUCCA | 451 | 3128-3150 |
| AD-1290109 | CACCGACACCU ACAGCAUCUA | 152 | 3142-3162 | UAGAUGCUGUAG GUGUCGGUGGC | 452 | 3140-3162 |
| AD-1290110 | UCUCGGGCUAC GGAGCUGCUA | 153 | 3159-3179 | UAGCAGCUCCGUA GCCCGAGAUG | 453 | 3157-3179 |
| AD-1290111 | GGCGGGAAAGG CGGGAAGAAA | 154 | 3179-3199 | UUUCUUCCCGCCU UUCCCGCCAG | 454 | 3177-3199 |
| AD-1290112 | GGGAAGAACAC CAUGAUGCGA | 155 | 3191-3211 | UCGCAUCAUGGU GUUCUUCCCGC | 455 | 3189-3211 |
| AD-1290113 | GAUGCGGUCCC ACGGCGUGUA | 156 | 3205-3225 | UACACGCCGUGGG ACCGCAUCAU | 456 | 3203-3225 |
| AD-1290114 | GGCGUGUCUGU GCUGGGCAUA | 157 | 3218-3238 | UAUGCCCAGCACA GACACGCCGU | 457 | 3216-3238 |
| AD-1290115 | GGCAUCUUCAA CCUGGAGAAA | 158 | 3233-3253 | UUUCUCCAGGUU GAAGAUGCCCA | 458 | 3231-3253 |
| AD-1290116 | AAGGAUGACAU GCUGUACAUA | 159 | 3251-3271 | UAUGUACAGCAU GUCAUCCUUCU | 459 | 3249-3271 |
| AD-1290117 | CUGUACAUCCU GGUUGGGCAA | 160 | 3263-3283 | UUGCCCAACCAGG AUGUACAGCA | 460 | 3261-3283 |
| AD-1290118 | GUUGGGCAGCA GGGAGAGGAA | 161 | 3275-3295 | UUCCUCUCCCUGC UGCCCAACCA | 461 | 3273-3295 |
| AD-1290119 | AGUACAAACCA GUUAAUCCAA | 162 | 3305-3325 | UUGGAUUAACUG GUUUGUACUGG | 462 | 3303-3325 |
| AD-1290120 | UUAAUCCAGAA AGUCUGCAUA | 163 | 3317-3337 | UAUGCAGACUUU CUGGAUUAACU | 463 | 3315-3337 |
| AD-1290121 | UGCAUUGGAGA GAACAAUGUA | 164 | 3332-3352 | UACAUUGUUCUC UCCAAUGCAGA | 464 | 3330-3352 |
| AD-1290122 | AAUGUGAUAGA AGAAGAAAUA | 165 | 3347-3367 | UAUUUCUUCUUC UAUCACAUUGU | 465 | 3345-3367 |
| AD-1290123 | AUCCGUGUGAA CAGAAGCGUA | 166 | 3365-3385 | UACGCUUCUGUUC ACACGGAUUU | 466 | 3363-3385 |
| AD-1290124 | GAAGCGUGCAU GAGUGGGCAA | 167 | 3378-3398 | UUGCCCACUCAUG CACGCUUCUG | 467 | 3376-3398 |
| AD-1290125 | CACCUACGUAU UUAAGAUGAA | 168 | 3424-3444 | UUCAUCUUAAAU ACGUAGGUGGC | 468 | 3422-3444 |
| AD-1290126 | UAAGAUGAAGG AUGGAGUGCA | 169 | 3436-3456 | UGCACUCCAUCCU UCAUCUUAAA | 469 | 3434-3456 |
| AD-1290127 | CUGAUCAUUGC AGCCGGAGGA | 170 | 3464-3484 | UCCUCCGGCUGCA AUGAUCAGGG | 470 | 3462-3484 |
| AD-1290128 | CCAAGACAGAC ACGUUCCACA | 171 | 3504-3524 | UGUGGAACGUGU CUGUCUUGGCC | 471 | 3502-3524 |
| AD-1290129 | AGAGAGACUGG AGAAUAACUA | 172 | 3526-3546 | UAGUUAUUCUCC AGUCUCUCUGG | 472 | 3524-3546 |
| AD-1290130 | GAAUAACUCCU CGGUUCUAGA | 173 | 3538-3558 | UCUAGAACCGAG GAGUUAUUCUC | 473 | 3536-3558 |
| AD-1290131 | AGGGCUAAACG GCAAUUCCGA | 174 | 3556-3576 | UCGGAAUUGCCG UUUAGCCCUAG | 474 | 3554-3576 |
| AD-1290132 | AUUCCGGAGCC GCAGGUGGUA | 175 | 3570-3590 | UACCACCUGCGGC UCCGGAAUUG | 475 | 3568-3590 |
| AD-1290133 | GUGGUGGAGGU GGCUGGAAUA | 176 | 3585-3605 | UAUUCCAGCCACC UCCACCACCU | 476 | 3583-3605 |
| AD-1290134 | UGGAAUGAUAA CACUUCCUUA | 177 | 3599-3619 | UAAGGAAGUGUU AUCAUUCCAGC | 477 | 3597-3619 |
| AD-1290135 | ACUUCCUUGCU CUGGGCCGGA | 178 | 3611-3631 | UCCGGCCCAGAGC AAGGAAGUGU | 478 | 3609-3631 |
| AD-1290136 | GGCCGGAAAAU CUUUGCAGGA | 179 | 3625-3645 | UCCUGCAAAGAU UUUCCGGCCCA | 479 | 3623-3645 |
| AD-1290137 | UGCAGGAGGGU GCCACCGGAA | 180 | 3639-3659 | UUCCGGUGGCACC CUCCUGCAAA | 480 | 3637-3659 |
| AD-1290138 | CCACCGGAGGA CAUUCCUGCA | 181 | 3651-3671 | UGCAGGAAUGUC CUCCGGUGGCA | 481 | 3649-3671 |
| AD-1290139 | AGGCGGAGGAU AUAUAGGCGA | 182 | 3751-3771 | UCGCCUAUAUAUC CUCCGCCUCC | 482 | 3749-3771 |
| AD-1290140 | AUGCAGCCUCA AACAAUGACA | 183 | 3774-3794 | UGUCAUUGUUUG AGGCUGCAUUG | 483 | 3772-3794 |
| AD-1290141 | GUUUCCUUCAU CAGUCCACUA | 184 | 3818-3838 | UAGUGGACUGAU GAAGGAAACCC | 484 | 3816-3838 |
| AD-1290142 | CACUGGGCAUC CUGUACACCA | 185 | 3834-3854 | UGGUGUACAGGA UGCCCAGUGGA | 485 | 3832-3854 |
| AD-1290143 | CAGCUUUAAAA GUGAUGGAAA | 186 | 3855-3875 | UUUCCAUCACUUU UAAAGCUGGG | 486 | 3853-3875 |
| AD-1290144 | UAAGCAUUAUC UAAACUGCAA | 187 | 3895-3915 | UUGCAGUUUAGA UAAUGCUUAAU | 487 | 3893-3915 |
| AD-1290145 | UGCAGUCACUG UGAGGUAGAA | 188 | 3911-3931 | UUCUACCUCACAG UGACUGCAGU | 488 | 3909-3931 |
| AD-1290146 | GUAGACGAAUG UCACAUGGAA | 189 | 3926-3946 | UUCCAUGUGACA UUCGUCUACCU | 489 | 3924-3946 |
| AD-1290147 | CCCUGAAAGCC ACAAGGUCAA | 190 | 3946-3966 | UUGACCUUGUGG CUUUCAGGGUC | 490 | 3944-3966 |
| AD-1290148 | AAGGUCAUCUG CUUCUGUGAA | 191 | 3959-3979 | UUCACAGAAGCA GAUGACCUUGU | 491 | 3957-3979 |
| AD-1290149 | UUCUGUGACCA CGGGACGGUA | 192 | 3971-3991 | UACCGUCCCGUGG UCACAGAAGC | 492 | 3969-3991 |
| AD-1290150 | GCUGGCUGAGG AUGGCGUCUA | 193 | 3991-4011 | UAGACGCCAUCCU CAGCCAGCAC | 493 | 3989-4011 |
| AD-1290151 | GUCUCCUGCAU UGUGUCACCA | 194 | 4007-4027 | UGGUGACACAAU GCAGGAGACGC | 494 | 4005-4027 |
| AD-1290152 | CCACACCUGCCA CUCUCGCUA | 195 | 4037-4057 | UAGCGAGAGUGG CAGGUGUGGCU | 495 | 4035-4057 |
| AD-1290153 | UCGCUGAUCCU CUCUGUGGUA | 196 | 4052-4072 | UACCACAGAGAG GAUCAGCGAGA | 496 | 4050-4072 |
| AD-1290154 | UCUGUGGUGAC CUCUGCCCUA | 197 | 4064-4084 | UAGGGCAGAGGU CACCACAGAGA | 497 | 4062-4084 |
| AD-1290155 | GUCCUGGCUUU CUCCGGCAUA | 198 | 409-4117 | UAUGCCGGAGAA AGCCAGGACCA | 498 | 4095-4117 |
| AD-1290156 | UCCGGCAUCAU GAUUGUGUAA | 199 | 4109-4129 | UUACACAAUCAU GAUGCCGGAGA | 499 | 4107-4129 |
| AD-1290157 | UACCGCCGGAA GCACCAGGAA | 200 | 4127-4147 | UUCCUGGUGCUUC CGGCGGUACA | 500 | 4125-4147 |
| AD-1290158 | CACCAGGAGCU GCAAGCCAUA | 201 | 4139-4159 | UAUGGCUUGCAG CUCCUGGUGCU | 501 | 4137-4159 |
| AD-1290159 | CAAGCCAUGCA GAUGGAGCUA | 202 | 4151-4171 | UAGCUCCAUCUGC AUGGCUUGCA | 502 | 4149-4171 |
| AD-1290160 | AUGGAGCUGCA GAGCCCUGAA | 203 | 4163-4183 | UUCAGGGCUCUGC AGCUCCAUCU | 503 | 4161-4183 |
| AD-1290161 | CCUGAGUACAA GCUGAGCAAA | 204 | 4178-4198 | UUUGCUCAGCUU GUACUCAGGGC | 504 | 4176-4198 |
| AD-1290162 | GCUCCGCACCUC GACCAUCAA | 205 | 4198-4218 | UUGAUGGUCGAG GUGCGGAGCUU | 505 | 4196-4218 |
| AD-1290163 | ACCAUCAUGAC CGACUACAAA | 206 | 4211-4231 | UUUGUAGUCGGU CAUGAUGGUCG | 506 | 4209-4231 |
| AD-1290164 | CAACUACUGCU UUGCUGGCAA | 207 | 4234-4254 | UUGCCAGCAAAGC AGUAGUUGGG | 507 | 4232-4254 |
| AD-1290165 | UGCUGGCAAGA CCUCCUCCAA | 208 | 4246-4266 | UUGGAGGAGGUC UUGCCAGCAAA | 508 | 4244-4266 |
| AD-1290166 | UCCUCCAUCAG UGACCUGAAA | 209 | 4259-4279 | UUUCAGGUCACU GAUGGAGGAGG | 509 | 4257-4279 |
| AD-1290167 | UGAAGGAGGUG CCGCGGAAAA | 210 | 4275-4295 | UUUUCCGCGGCAC CUCCUUCAGG | 510 | 4273-4295 |
| AD-1290168 | GAGGUGUAUGA AGGCCAGGUA | 211 | 4337-4357 | UACCUGGCCUUCA UACACCUCCC | 511 | 4335-4357 |
| AD-1290169 | GGUGUCCGGAA UGCCCAACGA | 212 | 4354-4374 | UCGUUGGGCAUU CCGGACACCUG | 512 | 4352-4374 |
| AD-1290170 | GCUGUGAAGAC GCUGCCUGAA | 213 | 4394-4414 | UUCAGGCAGCGUC UUCACAGCCA | 513 | 4392-4414 |
| AD-1290171 | UGUGCUCUGAA CAGGACGAAA | 214 | 4416-4436 | UUUCGUCCUGUUC AGAGCACACU | 514 | 4414-4436 |
| AD-1290172 | GGACGAACUGG AUUUCCUCAA | 215 | 4429-4449 | UUGAGGAAAUCC AGUUCGUCCUG | 515 | 4427-4449 |
| AD-1290173 | UUCCUCAUGGA AGCCCUGAUA | 216 | 4442-4462 | UAUCAGGGCUUCC AUGAGGAAAU | 516 | 4440-4462 |
| AD-1290174 | GAUCAUCAGCA AAUUCAACCA | 217 | 4459-4479 | UGGUUGAAUUUG CUGAUGAUCAG | 517 | 4457-4479 |
| AD-1290175 | CAACCACCAGA ACAUUGUUCA | 218 | 4474-4494 | UGAACAAUGUUC UGGUGGUUGAA | 518 | 4472-4494 |
| AD-1290176 | CGGUUCAUCCU GCUGGAGCUA | 219 | 4526-4546 | UAGCUCCAGCAGG AUGAACCGGG | 519 | 4524-4546 |
| AD-1290177 | AGACCUCAAGU CCUUCCUCCA | 220 | 4558-4578 | UGGAGGAAGGAC UUGAGGUCUCC | 520 | 4556-4578 |
| AD-1290178 | AUGCUGGACCU UCUGCACGUA | 221 | 4619-4639 | UACGUGCAGAAG GUCCAGCAUGG | 521 | 4617-4639 |
| AD-1290179 | UCGGGACAUUG CCUGUGGCUA | 222 | 4642-4662 | UAGCCACAGGCAA UGUCCCGAGC | 522 | 4640-4662 |
| AD-1290180 | GUGGCUGUCAG UAUUUGGAGA | 223 | 4656-4676 | UCUCCAAAUACUG ACAGCCACAG | 523 | 4654-4676 |
| AD-1290181 | UGGAGGAAAAC CACUUCAUCA | 224 | 4671-4691 | UGAUGAAGUGGU UUUCCUCCAAA | 524 | 4669-4691 |
| AD-1290182 | CCGAGACAUUG CUGCCAGAAA | 225 | 4693-4713 | UUUCUGGCAGCA AUGUCUCGGUG | 525 | 4691-4713 |
| AD-1290183 | UGCCAGAAACU GCCUCUUGAA | 226 | 4705-4725 | UUCAAGAGGCAG UUUCUGGCAGC | 526 | 4703-4725 |
| AD-1290184 | CCUCUUGACCU GUCCAGGCCA | 227 | 4717-4737 | UGGCCUGGACAG GUCAAGAGGCA | 527 | 4715-4737 |
| AD-1290185 | GCCCUGGAAGA GUGGCCAAGA | 228 | 4734-4754 | UCUUGGCCACUCU UCCAGGGCCU | 528 | 4732-4754 |
| AD-1290186 | GGCCAAGAUUG GAGACUUCGA | 229 | 4747-4767 | UCGAAGUCUCCAA UCUUGGCCAC | 529 | 4745-4767 |
| AD-1290187 | CUUCGGGAUGG CCCGAGACAA | 230 | 4762-4782 | UUGUCUCGGGCCA UCCCGAAGUC | 530 | 4760-4782 |
| AD-1290188 | CCGAGACAUCU ACAGGGCGAA | 231 | 4774-4794 | UUCGCCCUGUAGA UGUCUCGGGC | 531 | 4772-4794 |
| AD-1290189 | GAGCUACUAUA GAAAGGGAGA | 232 | 4792-4812 | UCUCCCUUUCUAU AGUAGCUCGC | 532 | 4790-4812 |
| AD-1290190 | GGGAGGCUGUG CCAUGCUGCA | 233 | 4807-4827 | UGCAGCAUGGCAC AGCCUCCCUU | 533 | 4805-4827 |
| AD-1290191 | AUGCUGCCAGU UAAGUGGAUA | 234 | 4820-4840 | UAUCCACUUAACU GGCAGCAUGG | 534 | 4818-4840 |
| AD-1290192 | GAGGCCUUCAU GGAAGGAAUA | 235 | 4847-4867 | UAUUCCUUCCAUG AAGGCCUCUG | 535 | 4845-4867 |
| AD-1290193 | GAAGGAAUAUU CACUUCUAAA | 236 | 4859-4879 | UUUAGAAGUGAA UAUUCCUUCCA | 536 | 4857-4879 |
| AD-1290194 | ACUUCUAAAAC AGACACAUGA | 237 | 4871-4891 | UCAUGUGUCUGU UUUAGAAGUGA | 537 | 4869-4891 |
| AD-1290195 | ACAUGGUCCUU UGGAGUGCUA | 238 | 4886-4906 | UAGCACUCCAAAG GACCAUGUGU | 538 | 4884-4906 |
| AD-1290196 | GUGCUGCUAUG GGAAAUCUUA | 239 | 4901-4921 | UAAGAUUUCCCA UAGCAGCACUC | 539 | 4899-4921 |
| AD-1290197 | GAAAUCUUUUC UCUUGGAUAA | 240 | 4913-4933 | UUAUCCAAGAGA AAAGAUUUCCC | 540 | 4911-4933 |
| AD-1290198 | CAAAAGCAACC AGGAAGUUCA | 241 | 4948-4968 | UGAACUUCCUGG UUGCUUUUGCU | 541 | 4946-4968 |
| AD-1290199 | GGAAGUUCUGG AGUUUGUCAA | 242 | 4960-4980 | UUGACAAACUCCA GAACUUCCUG | 542 | 4958-4980 |
| AD-1290200 | GUUUGUCACCA GUGGAGGCCA | 243 | 4972-4992 | UGGCCUCCACUGG UGACAAACUC | 543 | 4970-4992 |
| AD-1290201 | CCACCCAAGAA CUGCCCUGGA | 244 | 5000-5020 | UCCAGGGCAGUUC UUGGGUGGGU | 544 | 4998-5020 |
| AD-1290202 | UGCCCUGGGCC UGUAUACCGA | 245 | 5012-5032 | UCGGUAUACAGG CCCAGGGCAGU | 545 | 5010-5032 |
| AD-1290203 | GUAUACCGGAU AAUGACUCAA | 246 | 5024-5044 | UUGAGUCAUUAU CCGGUAUACAG | 546 | 5022-5044 |
| AD-1290204 | GCUGGCAACAU CAGCCUGAAA | 247 | 5046-5066 | UUUCAGGCUGAU GUUGCCAGCAC | 547 | 5044-5066 |
| AD-1290205 | AGCCUGAAGAC AGGCCCAACA | 248 | 5058-5078 | UGUUGGGCCUGU CUUCAGGCUGA | 548 | 5056-5078 |
| AD-1290206 | AACUUUGCCAU CAUUUUGGAA | 249 | 5075-5095 | UUCCAAAAUGAU GGCAAAGUUGG | 549 | 5073-5095 |
| AD-1290207 | GGAGAGGAUUG AAUACUGCAA | 250 | 5092-5112 | UUGCAGUAUUCA AUCCUCUCCAA | 550 | 5090-5112 |
| AD-1290208 | ACCCAGGACCC GGAUGUAAUA | 251 | 5111-5131 | UAUUACAUCCGG GUCCUGGGUGC | 551 | 5109-5131 |
| AD-1290209 | GAUGUAAUCAA CACCGCUUUA | 252 | 5123-5143 | UAAAGCGGUGUU GAUUACAUCCG | 552 | 5121-5143 |
| AD-1290210 | ACCGCUUUGCC GAUAGAAUAA | 253 | 5135-5155 | UUAUUCUAUCGG CAAAGCGGUGU | 553 | 5133-5155 |
| AD-1290211 | AUAGAAUAUGG UCCACUUGUA | 254 | 5147-5167 | UACAAGUGGACC AUAUUCUAUCG | 554 | 5145-5167 |
| AD-1290212 | CCACUUGUGGA AGAGGAAGAA | 255 | 5159-5179 | UUCUUCCUCUUCC ACAAGUGGAC | 555 | 5157-5179 |
| AD-1290213 | GGAAGAGAAAG UGCCUGUGAA | 256 | 5173-5193 | UUCACAGGCACUU UCUCUUCCUC | 556 | 5171-5193 |
| AD-1290214 | UGAGGCCCAAG GACCCUGAGA | 257 | 5190-5210 | UCUCAGGGUCCUU GGGCCUCACA | 557 | 5188-5210 |
| AD-1290215 | CCUCCUCUCCUG GUCUCUCAA | 258 | 5216-5236 | UUGAGAGACCAG GAGAGGAGGAA | 558 | 5214-5236 |
| AD-1290216 | UCUCUCAACAG GCAAAACGGA | 259 | 5229-5249 | UCCGUUUUGCCUG UUGAGAGACC | 559 | 5227-5249 |
| AD-1290217 | UCUGCCUACCA CCUCCUCUGA | 260 | 5281-5301 | UCAGAGGAGGUG GUAGGCAGAGG | 560 | 5279-5301 |
| AD-1290218 | CUCCUCUGGCA AGGCUGCAAA | 261 | 5293-5313 | UUUGCAGCCUUGC CAGAGGAGGU | 561 | 5291-5313 |
| AD-1290219 | CUGCAAAGAAA CCCACAGCUA | 262 | 5307-5327 | UAGCUGUGGGUU UCUUUGCAGCC | 562 | 5305-5327 |
| AD-1290220 | GCAGAGAUCUC UGUUCGAGUA | 263 | 5327-5347 | UACUCGAACAGA GAUCUCUGCAG | 563 | 5325-5347 |
| AD-1290221 | GUUCGAGUCCC UAGAGGGCCA | 264 | 5339-5359 | UGGCCCUCUAGGG ACUCGAACAG | 564 | 5337-5359 |
| AD-1290222 | GUGAAUAUGGC AUUCUCUCAA | 265 | 5378-5398 | UUGAGAGAAUGC CAUAUUCACGU | 565 | 5376-5398 |
| AD-1290223 | CUCUCAGUCCA ACCCUCCUUA | 266 | 5392-5412 | UAAGGAGGGUUG GACUGAGAGAA | 566 | 5390-5412 |
| AD-1290224 | CCUCCUUCGGA GUUGCACAAA | 267 | 5405-5425 | UUUGUGCAACUCC GAAGGAGGGU | 567 | 5403-5425 |
| AD-1290225 | CCACGGAUCCA GAAACAAGCA | 268 | 5428-5448 | UGCUUGUUUCUG GAUCCGUGGAC | 568 | 5426-5448 |
| AD-1290226 | CACCAGCUUGU GGAACCCAAA | 269 | 5449-5469 | UUUGGGUUCCAC AAGCUGGUGGG | 569 | 5447-5469 |
| AD-1290227 | GAACCCAACGU ACGGCUCCUA | 270 | 5461-5481 | UAGGAGCCGUAC GUUGGGUUCCA | 570 | 5459-5481 |
| AD-1290228 | UCCUGGUUUAC AGAGAAACCA | 271 | 5477-5497 | UGGUUUCUCUGU AAACCAGGAGC | 571 | 5475-5497 |
| AD-1290229 | UAAUCCUAUAG CAAAGAAGGA | 272 | 5509-5529 | UCCUUCUUUGCUA UAGGAUUAUU | 572 | 5507-5529 |
| AD-1290230 | AGAAGGAGCCA CACGACAGGA | 273 | 5523-5543 | UCCUGUCGUGUG GCUCCUUCUUU | 573 | 5521-5543 |
| AD-1290231 | AGGGAAGCUGU ACUGUCCCAA | 274 | 5559-5579 | UUGGGACAGUAC AGCUUCCCUCC | 574 | 5557-5579 |
| AD-1290232 | CCACCUAACGU UGCAACUGGA | 275 | 5576-5596 | UCCAGUUGCAACG UUAGGUGGGA | 575 | 5574-5596 |
| AD-1290233 | ACUGCUCCUAG AGCCCUCUUA | 276 | 5614-5634 | UAAGAGGGCUCU AGGAGCAGUGA | 576 | 5612-5634 |
| AD-1290234 | UCGCUGACUGC CAAUAUGAAA | 277 | 5633-5653 | UUUCAUAUUGGC AGUCAGCGAAG | 577 | 5631-5653 |
| AD-1290235 | AAUAUGAAGGA GGUACCUCUA | 278 | 5645-5665 | UAGAGGUACCUCC UUCAUAUUGG | 578 | 5643-5665 |
| AD-1290236 | GUACCUCUGUU CAGGCUACGA | 279 | 5657-5677 | UCGUAGCCUGAAC AGAGGUACCU | 579 | 5655-5677 |
| AD-1290237 | GCUACGUCACU UCCCUUGUGA | 280 | 5671-5691 | UCACAAGGGAAG UGACGUAGCCU | 580 | 5669-5691 |
| AD-1290238 | UUGUGGGAAUG UCAAUUACGA | 281 | 5686-5706 | UCGUAAUUGACA UUCCCACAAGG | 581 | 5684-5706 |
| AD-1290239 | GGCUACCAGCA ACAGGGCUUA | 282 | 5705-5725 | UAAGCCCUGUUGC UGGUAGCCGU | 582 | 5703-5725 |
| AD-1290240 | UUGCCCUUAGA AGCCGCUACA | 283 | 5723-5743 | UGUAGCGGCUUC UAAGGGCAAGC | 583 | 5721-5743 |
| AD-1290241 | UGGAGCUGGUC AUUACGAGGA | 284 | 5749-5769 | UCCUCGUAAUGAC CAGCUCCAGG | 584 | 5747-5769 |
| AD-1290242 | UUACGAGGAUA CCAUUCUGAA | 285 | 5761-5781 | UUCAGAAUGGUA UCCUCGUAAUG | 585 | 5759-5781 |
| AD-1290243 | CAUUCUGAAAA GCAAGAAUAA | 286 | 5773-5793 | UUAUUCUUGCUU UUCAGAAUGGU | 586 | 5771-5793 |
| AD-1290244 | AGAAUAGCAUG AACCAGCCUA | 287 | 5787-5807 | UAGGCUGGUUCA UGCUAUUCUUG | 587 | 5785-5807 |
| AD-1290245 | GAGCUCGGUCG CACACUCACA | 288 | 5814-5834 | UGUGAGUGUGCG ACCGAGCUCAG | 588 | 5812-5834 |
| AD-1290246 | ACACUCACUUC UCUUCCUUGA | 289 | 5826-5846 | UCAAGGAAGAGA AGUGAGUGUGC | 589 | 5824-5846 |
| AD-1290247 | GGGAUCCCUAA GACCGUGGAA | 290 | 5845-5865 | UUCCACGGUCUUA GGGAUCCCAA | 590 | 5843-5865 |
| AD-1290248 | ACCGUGGAGGA GAGAGAGGCA | 291 | 5857-5877 | UGCCUCUCUCUCC UCCACGGUCU | 591 | 5855-5877 |
| AD-1290249 | AGAGAGGCAAU GGCUCCUUCA | 292 | 5869-5889 | UGAAGGAGCCAU UGCCUCUCUCU | 592 | 5867-5889 |
| AD-1290250 | GCUCCUUCACA AACCAGAGAA | 293 | 5881-5901 | UUCUCUGGUUUG UGAAGGAGCCA | 593 | 5879-5901 |
| AD-1290251 | GAGACCAAAUG UCACGUUUUA | 294 | 5897-5917 | UAAAACGUGACA UUUGGUCUCUG | 594 | 5895-5917 |
| AD-1290252 | CACGUUUUGUU UUGUGCCAAA | 295 | 5909-5929 | UUUGGCACAAAA CAAAACGUGAC | 595 | 5907-5929 |
| AD-1290253 | GCCAACCUAUU UUGAAGUACA | 296 | 5924-5944 | UGUACUUCAAAA UAGGUUGGCAC | 596 | 5922-5944 |
| AD-1290254 | UGUAUUUUGAA AAUGCUUUAA | 297 | 5956-5976 | UUAAAGCAUUUU CAAAAUACAGC | 597 | 5954-5976 |
| AD-1290255 | UUUAGAAAGGU UUUGAGCAUA | 298 | 5972-5992 | UAUGCUCAAAACC UUUCUAAAGC | 598 | 5970-5992 |
| AD-1290256 | UUGAGCAUGGG UUCAUCCUAA | 299 | 5984-6004 | UUAGGAUGAACC CAUGCUCAAAA | 599 | 5982-6004 |
| AD-1290257 | CAUCCUAUUCU UUCGAAAGAA | 300 | 5997-6017 | UUCUUUCGAAAG AAUAGGAUGAA | 600 | 5995-6017 |
| AD-1290258 | AAUGAGUGAUA AAUACAAGGA | 301 | 6032-6052 | UCCUUGUAUUUA UCACUCAUUUU | 601 | 6030-6052 |
| AD-1290259 | AUACAAGGCCC AGAUGUGGUA | 302 | 6044-6064 | UACCACAUCUGGG CCUUGUAUUU | 602 | 6042-6064 |
| AD-1290260 | GAUGUGGUUGC AUAAGGUUUA | 303 | 6056-6076 | UAAACCUUAUGC AACCACAUCUG | 603 | 6054-6076 |
| AD-1290261 | UGCAUGUUUGU UGUAUACUUA | 304 | 6079-6099 | UAAGUAUACAAC AAACAUGCAUA | 604 | 6077-6099 |
| AD-1290262 | UUCCUUAUGCU UCUUUCAAAA | 305 | 6097-6117 | UUUUGAAAGAAG CAUAAGGAAGU | 605 | 6095-6117 |
| AD-1290263 | UUUCAAAUUGU GUGUGCUCUA | 306 | 6110-6130 | UAGAGCACACACA AUUUGAAAGA | 606 | 6108-6130 |
| AD-1290264 | CUGCUUCAAUG UAGUCAGAAA | 307 | 6128-6148 | UUUCUGACUACA UUGAAGCAGAG | 607 | 6126-6148 |
| AD-1290265 | AGUCAGAAUUA GCUGCUUCUA | 308 | 6140-6160 | UAGAAGCAGCUA AUUCUGACUAC | 608 | 6138-6160 |
| AD-1290266 | UGCUUCUAUGU UUCAUAGUUA | 309 | 6153-6173 | UAACUAUGAAAC AUAGAAGCAGC | 609 | 6151-6173 |
| AD-1290267 | AUGUUUCCUUG CCUUGUUGAA | 310 | 6183-6203 | UUCAACAAGGCA AGGAAACAUCU | 610 | 6181-6203 |
| AD-1290268 | UGUGGACAUGA GCCAUUUGAA | 311 | 6203-6223 | UUCAAAUGGCUC AUGUCCACAUC | 611 | 6201-6223 |
| AD-1290269 | ACGGAAAUAAA GGAGUUAUUA | 312 | 6234-6254 | UAAUAACUCCUU UAUUUCCGUUC | 612 | 6232-6254 |
| AD-1290270 | AGUUAUUUGUA AUGACUAAAA | 313 | 6247-6267 | UUUUAGUCAUUA CAAAUAACUCC | 613 | 6245-6267 |

TABLE 3

Modified Sense and Antisense Strand Sequences of Human ALK dsRNA Agents

| Duplex ID | Sense Sequence 5′ to 3′ | SEQ ID NO | Antisense Sequence 5′ to 3′ | SEQ ID NO | mRNA Target Sequence 5′ to 3′ | SEQ ID NO |
|---|---|---|---|---|---|---|
| AD-1289971 | gscsaga(Uhd)GfcGfAf Ufccagcggcsusa | 614 | VPusAfsgccGfcUfGfgau cGfcAfucugcscsu | 914 | AGGCAGATGCGAT CCAGCGGCTC | 1214 |
| AD-1289972 | csgsgug(Ghd)UfaGfC fAfgcugguacscsa | 615 | VPusGfsguaCfcAfGfcug cUfaCfcaccgscsu | 915 | AGCGGTGGTAGCA GCTGGTACCT | 1215 |
| AD-1289973 | gscsgcu(Ghd)AfuGfA fUfgggugaggsasa | 616 | VPusUfsccuCfaCfCfcau cAfuCfagcgcscsc | 916 | GGGCGCTGATGATG GGTGAGGAG | 1216 |
| AD-1289974 | usgsccu(Ghd)CfgAfA fCfucugaggasgsa | 617 | VPusCfsuccUfcAfGfagu uCfgCfaggcascsu | 917 | AGTGCCTGCGAACT CTGAGGAGC | 1217 |
| AD-1289975 | gsgsacg(Chd)UfgCfAf Afacuugcgcsasa | 618 | VPusUfsgcgCfaAfGfuuu gCfaGfcguccsusu | 918 | AAGGACGCTGCAA ACTTGCGCAG | 1218 |
| AD-1289976 | gscsugg(Ghd)AfuUfC fAfcgcccagasasa | 619 | VPusUfsucuGfgGfCfgug aAfuCfccagcscsc | 919 | GGGCTGGGATTCAC GCCCAGAAG | 1219 |
| AD-1289977 | gscscca(Ghd)AfaGfUf Ufcagcaggcsasa | 620 | VPusUfsgccUfgCfUfgaa cUfuCfugggcsgsu | 920 | ACGCCCAGAAGTTC AGCAGGCAG | 1220 |
| AD-1289978 | gscsaga(Chd)AfgUfCf Cfgaagccuuscsa | 621 | VPusGfsaagGfcUfUfcgg aCfuGfucugcscsu | 921 | AGGCAGACAGTCC GAAGCCTTCC | 1221 |
| AD-1289979 | csasgcg(Ghd)AfgAfG fAfuagcuugasgsa | 622 | VPusCfsucaAfgCfUfauc uCfuCfcgcugscsg | 922 | CGCAGCGGAGAGA TAGCTTGAGG | 1222 |
| AD-1289980 | usgsagg(Ghd)UfgCfG fCfaagacggcsasa | 623 | VPusUfsgccGfuCfUfugc gCfaCfccucasasg | 923 | CTTGAGGGTGCGCA AGACGGCAG | 1223 |
| AD-1289981 | gsgsgca(Ghd)AfaGfA fGfcuuggaggsasa | 624 | VPusUfsccuCfcAfAfgcu cUfuCfugcccsgsg | 924 | CCGGGCAGAAGAG CTTGGAGGAG | 1224 |
| AD-1289982 | gsasgcc(Ahd)AfaAfGf Gfaacgcaaasasa | 625 | VPusUfsuuuGfcGfUfucc uUfuUfggcucscsu | 925 | AGGAGCCAAAAGG AACGCAAAAG | 1225 |
| AD-1289983 | asasagg(Chd)GfgCfCf Afggacagcgsusa | 626 | VPusAfscgcUfgUfCfcug gCfcGfccuuususg | 926 | CAAAAGGCGGCCA GGACAGCGTG | 1226 |
| AD-1289984 | cscsgcc(Ghd)UfuCfUf Cfagccuuaasasa | 627 | VPusUfsuuaAfgGfCfuga gAfaCfggcggscsu | 927 | AGCCGCCGTTCTCA GCCTTAAAA | 1227 |
| AD-1289985 | cscsuua(Ahd)AfaGfUf Ufgcagagaususa | 628 | VPusAfsaucUfcUfGfcaa cUfuUfuaaggscsu | 928 | AGCCTTAAAAGTTG CAGAGATTG | 1228 |
| AD-1289986 | gsascgg(Uhd)AfcCfCf Afacugccacscsa | 629 | VPusGfsgugGfcAfGfuu ggGfuAfccgucscsu | 929 | AGGACGGTACCCA ACTGCCACCT | 1229 |
| AD-1289987 | csusgcc(Ahd)CfcUfCf Cfcuucaaccsasa | 630 | VPusUfsgguUfgAfAfgg gaGfgUfggcagsusu | 930 | AACTGCCACCTCCC TTCAACCAT | 1230 |
| AD-1289988 | uscsaac(Chd)AfuAfGf Ufaguuccucsusa | 631 | VPusAfsgagGfaAfCfuac uAfuGfguugasasg | 931 | CTTCAACCATAGTA GTTCCTCTG | 1231 |
| AD-1289989 | gsusucc(Uhd)CfuGfU fAfccgagcgcsasa | 632 | VPusUfsgcgCfuCfGfgua cAfgAfggaacsusa | 932 | TAGTTCCTCTGTAC CGAGCGCAG | 1232 |
| AD-1289990 | csgsagc(Ghd)CfaGfCf Gfagcuacagsasa | 633 | VPusUfscugUfaGfCfucg cUfgCfgcucgsgsu | 933 | ACCGAGCGCAGCG AGCTACAGAC | 1233 |
| AD-1289991 | gsgscuc(Ahd)AfgGfU fCfccagccagsusa | 634 | VPusAfscugGfcUfGfgga cCfuUfgagccsusc | 934 | GAGGCTCAAGGTCC CAGCCAGTG | 1234 |
| AD-1289992 | gscscag(Uhd)GfaGfCf Cfcagugugcsusa | 635 | VPusAfsgcaCfaCfUfggg cUfcAfcuggcsusg | 935 | CAGCCAGTGAGCCC AGTGTGCTT | 1235 |
| AD-1289993 | asgsugu(Ghd)CfuUfG fAfgugucucusgsa | 636 | VPusCfsagaGfaCfAfcuc aAfgCfacacusgsg | 936 | CCAGTGTGCTTGAG TGTCTCTGG | 1236 |
| AD-1289994 | gsgsucu(Ghd)UfuUfC fAfuuuagacuscsa | 637 | VPusGfsaguCfuAfAfaug aAfaCfagaccsusg | 937 | CAGGTCTGTTTCAT TTAGACTCC | 1237 |
| AD-1289995 | csusgcu(Chd)GfcCfUf Cfcgugcagususa | 638 | VPusAfsacuGfcAfCfgga gGfcGfagcagsgsa | 938 | TCCTGCTCGCCTCC GTGCAGTTG | 1238 |
| AD-1289996 | gsasaag(Chd)AfaGfAf Gfacuugcgcsgsa | 639 | VPusCfsgcgCfaAfGfucu cUfuGfcuuucscsc | 939 | GGGAAAGCAAGAG ACTTGCGCGC | 1239 |
| AD-1289997 | gscsgcg(Chd)AfcGfCf Afcaguccucsusa | 640 | VPusAfsgagGfaCfUfgug cGfuGfcgcgcsasa | 940 | TTGCGCGCACGCAC AGTCCTCTG | 1240 |
| AD-1289998 | uscscuc(Uhd)GfgAfG fAfucagguggsasa | 641 | VPusUfsccaCfcUfGfauc uCfcAfgaggascsu | 941 | AGTCCTCTGGAGAT CAGGTGGAA | 1241 |
| AD-1289999 | asgsgag(Chd)CfgCfUf Gfgguaccaasgsa | 642 | VPusCfsuugGfuAfCfcca gCfgGfcuccususc | 942 | GAAGGAGCCGCTG GGTACCAAGG | 1242 |
| AD-1290000 | gsusacc(Ahd)AfgGfA fCfuguucagasgsa | 643 | VPusCfsucuGfaAfCfagu cCfuUfgguacscsc | 943 | GGGTACCAAGGAC TGTTCAGAGC | 1243 |
| AD-1290001 | uscsaga(Ghd)CfcUfCf Ufucccaucuscsa | 644 | VPusGfsagaUfgGfGfaag aGfgCfucugasasc | 944 | GTTCAGAGCCTCTT CCCATCTCG | 1244 |
| AD-1290002 | cscsgga(Ghd)AfgCfAf Gfuguaaacgsgsa | 645 | VPusCfscguUfuAfCfacu gCfuCfuccggsgsc | 945 | GCCCGGAGAGCAG TGTAAACGGC | 1245 |
| AD-1290003 | gsgsgag(Chd)CfaUfCf Gfggcuccugsusa | 646 | VPusAfscagGfaGfCfccg aUfgGfcucccsasu | 946 | ATGGGAGCCATCG GGCTCCTGTG | 1246 |
| AD-1290004 | usgscug(Chd)UfuUfC fCfacggcagcsusa | 647 | VPusAfsgcuGfcCfGfugg aAfaGfcagcasgsc | 947 | GCTGCTGCTTTCCA CGGCAGCTG | 1247 |
| AD-1290005 | cscsacu(Chd)AfgCfUf Afcucgcgccsusa | 648 | VPusAfsggcGfcGfAfgua gCfuGfaguggscsu | 948 | AGCCACTCAGCTAC TCGCGCCTG | 1248 |
| AD-1290006 | csasgag(Ghd)AfaGfAf Gfucuggcagsusa | 649 | VPusAfscugCfcAfGfacu cUfuCfcucugscsa | 949 | TGCAGAGGAAGAG TCTGGCAGTT | 1249 |
| AD-1290007 | csusggc(Ahd)GfuUfG fAfcuucguggsusa | 650 | VPusAfsccaCfgAfAfguc aAfcUfgccagsasc | 950 | GTCTGGCAGTTGAC TTCGTGGTG | 1250 |
| AD-1290008 | ususcgu(Ghd)GfuGfC fCfcucgcucususa | 651 | VPusAfsagaGfcGfAfggg cAfcCfacgaasgsu | 951 | ACTTCGTGGTGCCC TCGCTCTTC | 1251 |
| AD-1290009 | uscsgcu(Chd)UfuCfCf Gfugucuacgscsa | 652 | VPusGfscguAfgAfCfacg gAfaGfagcgasgsg | 952 | CCTCGCTCTTCCGT GTCTACGCC | 1252 |
| AD-1290010 | gsuscua(Chd)GfcCfCf Gfggaccuacsusa | 653 | VPusAfsguaGfgUfCfccg gGfcGfuagacsasc | 953 | GTGTCTACGCCCGG GACCTACTG | 1253 |
| AD-1290011 | gsasccu(Ahd)CfuGfCf Ufgccaccauscsa | 654 | VPusGfsaugGfuGfGfcag cAfgUfaggucscsc | 954 | GGGACCTACTGCTG CCACCATCC | 1254 |
| AD-1290012 | csuscgg(Ahd)GfcUfG fAfaggcuggcsasa | 655 | VPusUfsgccAfgCfCfuuc aGfcUfccgagsgsa | 955 | TCCTCGGAGCTGAA GGCTGGCAG | 1255 |
| AD-1290013 | gscsugu(Chd)CfaGfGf Gfugcugaagsgsa | 656 | VPusCfscuuCfaGfCfacc cUfgGfacagcsgsu | 956 | ACGCTGTCCAGGGT GCTGAAGGG | 1256 |
| AD-1290014 | csgsugc(Chd)AfaGfCf Afguuggugcsusa | 657 | VPusAfsgcaCfcAfAfcug cUfuGfgcacgscsc | 957 | GGCGTGCCAAGCA GTTGGTGCTG | 1257 |
| AD-1290015 | ususggu(Ghd)CfuGfG fAfgcugggcgsasa | 658 | VPusUfscgcCfcAfGfcuc cAfgCfaccaascsu | 958 | AGTTGGTGCTGGAG CTGGGCGAG | 1258 |
| AD-1290016 | gsasggc(Ghd)AfuCfU fUfggaggguusgsa | 659 | VPusCfsaacCfcUfCfcaa gAfuCfgccucscsu | 959 | AGGAGGCGATCTTG GAGGGTTGC | 1259 |
| AD-1290017 | csusgcu(Chd)CfaGfUf Ufcaaucucasgsa | 660 | VPusCfsugaGfaUfUfgaa cUfgGfagcagscsc | 960 | GGCTGCTCCAGTTC AATCTCAGC | 1260 |
| AD-1290018 | csasgcg(Ahd)GfcUfGf Ufucaguuggsusa | 661 | VPusAfsccaAfcUfGfaac aGfcUfcgcugsasg | 961 | CTCAGCGAGCTGTT CAGTTGGTG | 1261 |
| AD-1290019 | ususggu(Ghd)GfaUfU fCfgccaaggcsgsa | 662 | VPusCfsgccUfuGfGfcga aUfcCfaccaascsu | 962 | AGTTGGTGGATTCG CCAAGGCGA | 1262 |
| AD-1290020 | gsgsgcg(Ahd)CfuGfA fGfgauccgccsusa | 663 | VPusAfsggcGfgAfUfccu cAfgUfcgcccsusu | 963 | AAGGGCGACTGAG GATCCGCCTG | 1263 |
| AD-1290021 | asusccg(Chd)CfuGfAf Ufgcccgagasasa | 664 | VPusUfsucuCfgGfGfcau cAfgGfcggauscsc | 964 | GGATCCGCCTGATG CCCGAGAAG | 1264 |
| AD-1290022 | cscsgag(Ahd)AfgAfA fGfgc gucggasasa | 665 | VPusUfsuccGfaCfGfccu uCfuUfcucggsgsc | 965 | GCCCGAGAAGAAG GCGTCGGAAG | 1265 |
| AD-1290023 | gsgsgca(Ghd)AfgAfG fGfgaaggcugsusa | 666 | VPusAfscagCfcUfUfccc uCfuCfugcccsasc | 966 | GTGGGCAGAGAGG GAAGGCTGTC | 1266 |
| AD-1290024 | gsgscug(Uhd)CfcGfCf Gfgcaauucgscsa | 667 | VPusGfscgaAfuUfGfccg cGfgAfcagccsusu | 967 | AAGGCTGTCCGCGG CAATTCGCG | 1267 |
| AD-1290025 | cscsuuc(Uhd)CfuUfCf Cfagaucuucsgsa | 668 | VPusCfsgaaGfaUfCfugg aAfgAfgaaggscsg | 968 | CGCCTTCTCTTCCA GATCTTCGG | 1268 |
| AD-1290026 | ascsugg(Uhd)CfaUfAf Gfcuccuuggsasa | 669 | VPusUfsccaAfgGfAfgcu aUfgAfccaguscsc | 969 | GGACTGGTCATAGC TCCTTGGAA | 1269 |
| AD-1290027 | cscsuug(Ghd)AfaUfCf Afccaacaaascsa | 670 | VPusGfsuuuGfuUfGfgu gaUfuCfcaaggsasg | 970 | CTCCTTGGAATCAC CAACAAACA | 1270 |
| AD-1290028 | csasaac(Ahd)UfgCfCf Ufucuccuucsusa | 671 | VPusAfsgaaGfgAfGfaag gCfaUfguuugsusu | 971 | AACAAACATGCCTT CTCCTTCTC | 1271 |
| AD-1290029 | csusucu(Chd)CfuGfAf Ufuauuuuacsasa | 672 | VPusUfsguaAfaAfUfaau cAfgGfagaagsgsa | 972 | TCCTTCTCCTGATT ATTTTACAT | 1272 |
| AD-1290030 | ususuua(Chd)AfuGfG fAfaucucaccsusa | 673 | VPusAfsgguGfaGfAfuuc cAfuGfuaaaasusa | 973 | TATTTTACATGGAA TCTCACCTG | 1273 |
| AD-1290031 | uscsuca(Chd)CfuGfGf Afuaaugaaasgsa | 674 | VPusCfsuuuCfaUfUfauc cAfgGfugagasusu | 974 | AATCTCACCTGGAT AATGAAAGA | 1274 |
| AD-1290032 | asusgaa(Ahd)GfaCfUf Cfcuucccuususa | 675 | VPusAfsaagGfgAfAfgga gUfcUfuucaususa | 975 | TAATGAAAGACTCC TTCCCTTTC | 1275 |
| AD-1290033 | ususccc(Uhd)UfuCfCf Ufgucucaucsgsa | 676 | VPusCfsgauGfaGfAfcag gAfaAfgggaasgsg | 976 | CCTTCCCTTTCCTGT CTCATCGC | 1276 |
| AD-1290034 | csasucg(Chd)AfgCfCf Gfauauggucsusa | 677 | VPusAfsgacCfaUfAfucg gCfuGfcgaugsasg | 977 | CTCATCGCAGCCGA TATGGTCTG | 1277 |
| AD-1290035 | gsgsagu(Ghd)CfaGfCf Ufuugacuucscsa | 678 | VPusGfsgaaGfuCfAfaag cUfgCfacuccsasg | 978 | CTGGAGTGCAGCTT TGACTTCCC | 1278 |
| AD-1290036 | cscsacu(Ghd)CfaUfGf Afccucaggasasa | 679 | VPusUfsuccUfgAfGfguc aUfgCfaguggsasg | 979 | CTCCACTGCATGAC CTCAGGAAC | 1279 |
| AD-1290037 | csasgga(Ahd)CfcAfGf Afgcugguccsusa | 680 | VPusAfsggaCfcAfGfcuc uGfgUfuccugsasg | 980 | CTCAGGAACCAGA GCTGGTCCTG | 1280 |
| AD-1290038 | uscscca(Ghd)AfuGfGf Afcuugcuggsasa | 681 | VPusUfsccaGfcAfAfguc cAfuCfugggasgsg | 981 | CCTCCCAGATGGAC TTGCTGGAT | 1281 |
| AD-1290039 | asgsagc(Ghd)UfuCfUf Afaggagaugscsa | 682 | VPusGfscauCfuCfCfuua gAfaCfgcucusgsc | 982 | GCAGAGCGTTCTAA GGAGATGCC | 1282 |
| AD-1290040 | asusgcc(Chd)AfgAfGf Gfcuccuuucsusa | 683 | VPusAfsgaaAfgGfAfgcc uCfuGfggcauscsu | 983 | AGATGCCCAGAGG CTCCTTTCTC | 1283 |
| AD-1290041 | uscscuu(Uhd)CfuCfCf Ufucucaacascsa | 684 | VPusGfsuguUfgAfGfaag gAfgAfaaggasgsc | 984 | GCTCCTTTCTCCTTC TCAACACC | 1284 |
| AD-1290042 | asgscug(Ahd)CfuCfCf Afagcacaccsasa | 685 | VPusUfsgguGfuGfCfuu ggAfgUfcagcusgsa | 985 | TCAGCTGACTCCAA GCACACCAT | 1285 |
| AD-1290043 | gscsaca(Chd)CfaUfCf Cfugaguccgsusa | 686 | VPusAfscggAfcUfCfagg aUfgGfugugcsusu | 986 | AAGCACACCATCCT GAGTCCGTG | 1286 |
| AD-1290044 | gsasguc(Chd)GfuGfG fAfugaggagcsasa | 687 | VPusUfsgcuCfcUfCfauc cAfcGfgacucsasg | 987 | CTGAGTCCGTGGAT GAGGAGCAG | 1287 |
| AD-1290045 | csasgca(Ghd)UfgAfGf Cfacugcacascsa | 688 | VPusGfsuguGfcAfGfugc uCfaCfugcugscsu | 988 | AGCAGCAGTGAGC ACTGCACACT | 1288 |
| AD-1290046 | usgscac(Ahd)CfuGfGf Cfcgucucggsusa | 689 | VPusAfsccgAfgAfCfggc cAfgUfgugcasgsu | 989 | ACTGCACACTGGCC GTCTCGGTG | 1289 |
| AD-1290047 | uscsggu(Ghd)CfaCfAf Gfgcaccugcsasa | 690 | VPusUfsgcaGfgUfGfccu gUfgCfaccgasgsa | 990 | TCTCGGTGCACAGG CACCTGCAG | 1290 |
| AD-1290048 | csuscug(Ghd)AfaGfG fUfacauugccscsa | 691 | VPusGfsggcAfaUfGfuac cUfuCfcagagsgsg | 991 | CCCTCTGGAAGGTA CATTGCCCA | 1291 |
| AD-1290049 | gscsugc(Ahd)AfgAfG fAfgauccuccsusa | 692 | VPusAfsggaGfgAfUfcuc uCfuUfgcagcscsu | 992 | AGGCTGCAAGAGA GATCCTCCTG | 1292 |
| AD-1290050 | asusccu(Chd)CfuGfAf Ufgcccacucscsa | 693 | VPusGfsgagUfgGfGfcau cAfgGfaggauscsu | 993 | AGATCCTCCTGATG CCCACTCCA | 1293 |
| AD-1290051 | csasggg(Ahd)AfgCfA fUfgguuggacsasa | 694 | VPusUfsgucCfaAfCfcau gCfuUfcccugsgsa | 994 | TCCAGGGAAGCAT GGTTGGACAG | 1294 |
| AD-1290052 | ususgga(Chd)AfgUfG fCfuccagggasasa | 695 | VPusUfsuccCfuGfGfagc aCfuGfuccaascsc | 995 | GGTTGGACAGTGCT CCAGGGAAG | 1295 |
| AD-1290053 | cscsagg(Ghd)AfaGfAf Afucgggcguscsa | 696 | VPusGfsacgCfcCfGfauu cUfuCfccuggsasg | 996 | CTCCAGGGAAGAA TCGGGCGTCC | 1296 |
| AD-1290054 | cscsaga(Chd)AfaCfCf Cfauuucgagsusa | 697 | VPusAfscucGfaAfAfugg gUfuGfucuggsasc | 997 | GTCCAGACAACCCA TTTCGAGTG | 1297 |
| AD-1290055 | uscsgag(Uhd)GfgCfCf Cfuggaauacsasa | 698 | VPusUfsguaUfuCfCfagg gCfcAfcucgasasa | 998 | TTTCGAGTGGCCCT GGAATACAT | 1298 |
| AD-1290056 | gsgsaau(Ahd)CfaUfCf Ufccaguggasasa | 699 | VPusUfsuccAfcUfGfgag aUfgUfauuccsasg | 999 | CTGGAATACATCTC CAGTGGAAA | 1299 |
| AD-1290057 | csasgug(Ghd)AfaAfCf Cfgcagcuugsusa | 700 | VPusAfscaaGfcUfGfcgg uUfuCfcacugsgsa | 1000 | TCCAGTGGAAACCG CAGCTTGTC | 1300 |
| AD-1290058 | csusgca(Ghd)UfgGfA fCfuucuuugcscsa | 701 | VPusGfsgcaAfaGfAfagu cCfaCfugcagsasc | 1001 | GTCTGCAGTGGACT TCTTTGCCC | 1301 |
| AD-1290059 | cscsuga(Ahd)GfaAfCf Ufgcagugaasgsa | 702 | VPusCfsuucAfcUfGfcag uUfcUfucaggsgsc | 1002 | GCCCTGAAGAACTG CAGTGAAGG | 1302 |
| AD-1290060 | gsgscuc(Chd)AfaGfAf Ufggcccugcsasa | 703 | VPusUfsgcaGfgGfCfcau cUfuGfgagccsusg | 1003 | CAGGCTCCAAGATG GCCCTGCAG | 1303 |
| AD-1290061 | csusccu(Uhd)CfaCfUf Ufguuggaausgsa | 704 | VPusCfsauuCfcAfAfcaa gUfgAfaggagscsu | 1004 | AGCTCCTTCACTTG TTGGAATGG | 1304 |
| AD-1290062 | ascsagu(Chd)CfuCfCf Afgcuugggcsasa | 705 | VPusUfsgccCfaAfGfcug gAfgGfacuguscsc | 1005 | GGACAGTCCTCCAG CTTGGGCAG | 1305 |
| AD-1290063 | csusgug(Ahd)CfuUfC fCfaccaggacsusa | 706 | VPusAfsgucCfuGfGfugg aAfgUfcacagsgsc | 1006 | GCCTGTGACTTCCA CCAGGACTG | 1306 |
| AD-1290064 | csasgga(Chd)UfgUfGf Cfccagggagsasa | 707 | VPusUfscucCfcUfGfggc aCfaGfuccugsgsu | 1007 | ACCAGGACTGTGCC CAGGGAGAA | 1307 |
| AD-1290065 | csasggg(Ahd)GfaAfG fAfugagagccsasa | 708 | VPusUfsggcUfcUfCfauc uUfcUfcccugsgsg | 1008 | CCCAGGGAGAAGA TGAGAGCCAG | 1308 |
| AD-1290066 | asgscca(Ghd)AfuGfUf Gfccggaaacsusa | 709 | VPusAfsguuUfcCfGfgca cAfuCfuggcuscsu | 1009 | AGAGCCAGATGTG CCGGAAACTG | 1309 |
| AD-1290067 | csgsgaa(Ahd)CfuGfCf Cfuguggguususa | 710 | VPusAfsaacCfcAfCfagg cAfgUfuuccgsgsc | 1010 | GCCGGAAACTGCCT GTGGGTTTT | 1310 |
| AD-1290068 | usgscaa(Chd)UfuUfGf Afagauggcususa | 711 | VPusAfsagcCfaUfCfuuc aAfaGfuugcasgsu | 1011 | ACTGCAACTTTGAA GATGGCTTC | 1311 |
| AD-1290069 | gsasugg(Chd)UfuCfU fGfuggcuggascsa | 712 | VPusGfsuccAfgCfCfaca gAfaGfccaucsusu | 1012 | AAGATGGCTTCTGT GGCTGGACC | 1312 |
| AD-1290070 | ascscca(Ahd)GfgCfAf Cfacugucacscsa | 713 | VPusGfsgugAfcAfGfug ugCfcUfuggguscsc | 1013 | GGACCCAAGGCAC ACTGTCACCC | 1313 |
| AD-1290071 | ascsucc(Uhd)CfaAfUf Gfgcaggucasgsa | 714 | VPusCfsugaCfcUfGfcca uUfgAfggagusgsu | 1014 | ACACTCCTCAATGG CAGGTCAGG | 1314 |
| AD-1290072 | gsasccc(Uhd)AfaAfGf Gfaugcccggsusa | 715 | VPusAfsccgGfgCfAfucc uUfuAfgggucscsu | 1015 | AGGACCCTAAAGG ATGCCCGGTT | 1315 |
| AD-1290073 | gscsccg(Ghd)UfuCfCf Afggaccaccsasa | 716 | VPusUfsgguGfgUfCfcug gAfaCfcgggcsasu | 1016 | ATGCCCGGTTCCAG GACCACCAA | 1316 |
| AD-1290074 | gsascca(Chd)CfaAfGf Afccaugcucsusa | 717 | VPusAfsgagCfaUfGfguc uUfgGfuggucscsu | 1017 | AGGACCACCAAGA CCATGCTCTA | 1317 |
| AD-1290075 | usgscuc(Uhd)AfuUfG fCfucaguaccsasa | 718 | VPusUfsgguAfcUfGfagc aAfuAfgagcasusg | 1018 | CATGCTCTATTGCT CAGTACCAC | 1318 |
| AD-1290076 | gscsuuc(Uhd)GfaAfA fGfugcuacagsusa | 719 | VPusAfscugUfaGfCfacu uUfcAfgaagcsgsg | 1019 | CCGCTTCTGAAAGT GCTACAGTG | 1319 |
| AD-1290077 | cscsagu(Ghd)CfuAfCf Gfuuuccugcsasa | 720 | VPusUfsgcaGfgAfAfacg uAfgCfacuggsusc | 1020 | GACCAGTGCTACGT TTCCTGCAC | 1320 |
| AD-1290078 | ascscga(Uhd)CfaAfGf Afgcucuccasusa | 721 | VPusAfsuggAfgAfGfcuc uUfgAfucggusgsc | 1021 | GCACCGATCAAGA GCTCTCCATG | 1321 |
| AD-1290079 | csuscuc(Chd)AluGfUf Gfagcuccgasasa | 722 | VPusUfsucgGfaGfCfuca cAfuGfgagagscsu | 1022 | AGCTCTCCATGTGA GCTCCGAAT | 1322 |
| AD-1290080 | gscsucc(Ghd)AfaUfGf Ufccuggcucsasa | 723 | VPusUfsgagCfcAfGfgac aUfuCfggagcsusc | 1023 | GAGCTCCGAATGTC CTGGCTCAT | 1323 |
| AD-1290081 | usgsgcu(Chd)AfuUfC fGfuggagucususa | 724 | VPusAfsagaCfuCfCfacg aAfuGfagccasgsg | 1024 | CCTGGCTCATTCGT GGAGTCTTG | 1324 |
| AD-1290082 | gsasaac(Ghd)UfgUfCf Cfuuggugcusasa | 725 | VPusUfsagcAfcCfAfagg aCfaCfguuucscsc | 1025 | GGGAAACGTGTCCT TGGTGCTAG | 1325 |
| AD-1290083 | gsusgcu(Ahd)GfuGfG fAfgaacaaaascsa | 726 | VPusGfsuuuUfgUfUfcuc cAfcUfagcacscsa | 1026 | TGGTGCTAGTGGAG AACAAAACC | 1326 |
| AD-1290084 | gsgsgaa(Ghd)GfaGfCf Afaggcaggasusa | 727 | VPusAfsuccUfgCfCfuug cUfcCfuucccsgsg | 1027 | CCGGGAAGGAGCA AGGCAGGATG | 1327 |
| AD-1290085 | gsgscag(Ghd)AfuGfG fUfcuggcaugsusa | 728 | VPusAfscauGfcCfAfgac cAfuCfcugccsusu | 1028 | AAGGCAGGATGGT CTGGCATGTC | 1328 |
| AD-1290086 | csgsccu(Ahd)UfgAfA fGfgcuugagcscsa | 729 | VPusGfsgcuCfaAfGfccu uCfaUfaggcgsgsc | 1029 | GCCGCCTATGAAGG CTTGAGCCT | 1329 |
| AD-1290087 | ususgag(Chd)CfuGfU fGfgcaguggasusa | 730 | VPusAfsuccAfcUfGfcca cAfgGfcucaasgsc | 1030 | GCTTGAGCCTGTGG CAGTGGATG | 1330 |
| AD-1290088 | csasgug(Ghd)AfuGfG fUfguugccucsusa | 731 | VPusAfsgagGfcAfAfcac cAfuCfcacugscsc | 1031 | GGCAGTGGATGGT GTTGCCTCTC | 1331 |
| AD-1290089 | csuscga(Uhd)GfuGfU fCfugacaggususa | 732 | VPusAfsaccUfgUfCfaga cAfcAfucgagsgsa | 1032 | TCCTCGATGTGTCT GACAGGTTC | 1332 |
| AD-1290090 | csusggc(Uhd)GfcAfG fAfuggucgcasusa | 733 | VPusAfsugcGfaCfCfauc uGfcAfgccagsasa | 1033 | TTCTGGCTGCAGAT GGTCGCATG | 1333 |
| AD-1290091 | asuscca(Ghd)AfgCfCf Afucguggcususa | 734 | VPusAfsagcCfaCfGfaug gCfuCfuggauscsc | 1034 | GGATCCAGAGCCAT CGTGGCTTT | 1334 |
| AD-1290092 | gsusggc(Uhd)UfuUfG fAfcaauaucuscsa | 735 | VPusGfsagaUfaUfUfguc aAfaAfgccacsgsa | 1035 | TCGTGGCTTTTGAC AATATCTCC | 1335 |
| AD-1290093 | asasuau(Chd)UfcCfAf Ufcagccuggsasa | 736 | VPusUfsccaGfgCfUfgau gGfaGfauauusgsu | 1036 | ACAATATCTCCATC AGCCTGGAC | 1336 |
| AD-1290094 | asgsccu(Ghd)GfaCfUf Gfcuaccucascsa | 737 | VPusGfsugaGfgUfAfgca gUfcCfaggcusgsa | 1037 | TCAGCCTGGACTGC TACCTCACC | 1337 |
| AD-1290095 | usasccu(Chd)AfcCfAf Ufuagcggagsasa | 738 | VPusUfscucCfgCfUfaau gGfuGfagguasgsc | 1038 | GCTACCTCACCATT AGCGGAGAG | 1338 |
| AD-1290096 | gsgsaca(Ahd)GfaUfCf Cfugcagaausasa | 739 | VPusUfsauuCfuGfCfagg aUfcUfuguccsusc | 1039 | GAGGACAAGATCC TGCAGAATAC | 1339 |
| AD-1290097 | asgsaau(Ahd)CfaGfCf Afcccaaaucsasa | 740 | VPusUfsgauUfuGfGfgu gcUfgUfauucusgsc | 1040 | GCAGAATACAGCA CCCAAATCAA | 1340 |
| AD-1290098 | cscsaaa(Uhd)CfaAfGf Afaaccuguususa | 741 | VPusAfsaacAfgGfUfuuc uUfgAfuuuggsgsu | 1041 | ACCCAAATCAAGA AACCTGTTTG | 1341 |
| AD-1290099 | cscsugu(Uhd)UfgAfG fAfgaaacccasasa | 742 | VPusUfsuggGfuUfUfcuc uCfaAfacaggsusu | 1042 | AACCTGTTTGAGAG AAACCCAAA | 1342 |
| AD-1290100 | cscscaa(Ahd)CfaAfGf Gfagcugaaascsa | 743 | VPusGfsuuuCfaGfCfucc uUfgUfuugggsusu | 1043 | AACCCAAACAAGG AGCTGAAACC | 1343 |
| AD-1290101 | gsasaaa(Uhd)UfcAfCf Cfaagacagascsa | 744 | VPusGfsucuGfuCfUfugg uGfaAfuuuucscsc | 1044 | GGGAAAATTCACC AAGACAGACC | 1344 |
| AD-1290102 | csusuug(Ahd)CfcCfUf Afcaguucaususa | 745 | VPusAfsaugAfaCfUfgua gGfgUfcaaagsasu | 1045 | ATCTTTGACCCTAC AGTTCATTG | 1345 |
| AD-1290103 | asgsuuc(Ahd)UfuGfG fCfuguucaccsasa | 746 | VPusUfsgguGfaAfCfagc cAfaUfgaacusgsu | 1046 | ACAGTTCATTGGCT GTTCACCAC | 1346 |
| AD-1290104 | gscsaca(Ghd)UfgCfAf Afcaacgccusasa | 747 | VPusUfsaggCfgUfUfguu gCfaCfugugcscsu | 1047 | AGGCACAGTGCAA CAACGCCTAC | 1347 |
| AD-1290105 | csusacc(Ahd)GfaAfCf Ufccaaccugsasa | 748 | VPusUfscagGfuUfGfgag uUfcUfgguagsgsc | 1048 | GCCTACCAGAACTC CAACCTGAG | 1348 |
| AD-1290106 | csasacc(Uhd)GfaGfCf Gfuggaggugsgsa | 749 | VPusCfscacCfuCfCfacg cUfcAfgguugsgsa | 1049 | TCCAACCTGAGCGT GGAGGTGGG | 1349 |
| AD-1290107 | csasucc(Ahd)GfaUfCf Ufggaaggugscsa | 750 | VPusGfscacCfuUfCfcag aUfcUfggaugscsc | 1050 | GGCATCCAGATCTG GAAGGTGCC | 1350 |
| AD-1290108 | gsasagg(Uhd)GfcCfAf Gfccaccgacsasa | 751 | VPusUfsgucGfgUfGfgcu gGfcAfccuucscsa | 1051 | TGGAAGGTGCCAG CCACCGACAC | 1351 |
| AD-1290109 | csasccg(Ahd)CfaCfCf Ufacagcaucsusa | 752 | VPusAfsgauGfcUfGfuag gUfgUfcggugsgsc | 1052 | GCCACCGACACCTA CAGCATCTC | 1352 |
| AD-1290110 | uscsucg(Ghd)GfcUfA fCfggagcugcsusa | 753 | VPusAfsgcaGfcUfCfcgu aGfcCfcgagasusg | 1053 | CATCTCGGGCTACG GAGCTGCTG | 1353 |
| AD-1290111 | gsgscgg(Ghd)AfaAfG fGfcgggaagasasa | 754 | VPusUfsucuUfcCfCfgcc uUfuCfccgccsasg | 1054 | CTGGCGGGAAAGG CGGGAAGAAC | 1354 |
| AD-1290112 | gsgsgaa(Ghd)AfaCfAf Cfcaugaugcsgsa | 755 | VPusCfsgcaUfcAfUfggu gUfuCfuucccsgsc | 1055 | GCGGGAAGAACAC CATGATGCGG | 1355 |
| AD-1290113 | gsasugc(Ghd)GfuCfCf Cfacggcgugsusa | 756 | VPusAfscacGfcCfGfugg gAfcCfgcaucsasu | 1056 | ATGATGCGGTCCCA CGGCGTGTC | 1356 |
| AD-1290114 | gsgscgu(Ghd)UfcUfG fUfgcugggcasusa | 757 | VPusAfsugcCfcAfGfcac aGfaCfacgccsgsu | 1057 | ACGGCGTGTCTGTG CTGGGCATC | 1357 |
| AD-1290115 | gsgscau(Chd)UfuCfAf Afccuggagasasa | 758 | VPusUfsucuCfcAfGfguu gAfaGfaugccscsa | 1058 | TGGGCATCTTCAAC CTGGAGAAG | 1358 |
| AD-1290116 | asasgga(Uhd)GfaCfAf Ufgcuguacasusa | 759 | VPusAfsuguAfcAfGfcau gUfcAfuccuuscsu | 1059 | AGAAGGATGACAT GCTGTACATC | 1359 |
| AD-1290117 | csusgua(Chd)AfuCfCf Ufgguugggcsasa | 760 | VPusUfsgccCfaAfCfcag gAfuGfuacagscsa | 1060 | TGCTGTACATCCTG GTTGGGCAG | 1360 |
| AD-1290118 | gsusugg(Ghd)CfaGfC fAfgggagaggsasa | 761 | VPusUfsccuCfuCfCfcug cUfgCfccaacscsa | 1061 | TGGTTGGGCAGCAG GGAGAGGAC | 1361 |
| AD-1290119 | asgsuac(Ahd)AfaCfCf Afguuaauccsasa | 762 | VPusUfsggaUfuAfAfcug gUfuUfguacusgsg | 1062 | CCAGTACAAACCA GTTAATCCAG | 1362 |
| AD-1290120 | ususaau(Chd)CfaGfAf Afagucugcasusa | 763 | VPusAfsugcAfgAfCfuuu cUfgGfauuaascsu | 1063 | AGTTAATCCAGAAA GTCTGCATT | 1363 |
| AD-1290121 | usgscau(Uhd)GfgAfG fAfgaacaaugsusa | 764 | VPusAfscauUfgUfUfcuc uCfcAfaugcasgsa | 1064 | TCTGCATTGGAGAG AACAATGTG | 1364 |
| AD-1290122 | asasugu(Ghd)AfuAfG fAfagaagaaasusa | 765 | VPusAfsuuuCfuUfCfuuc uAfuCfacauusgsu | 1065 | ACAATGTGATAGA AGAAGAAATC | 1365 |
| AD-1290123 | asusccg(Uhd)GfuGfA fAfcagaagcgsusa | 766 | VPusAfscgcUfuCfUfguu cAfcAfcggaususu | 1066 | AAATCCGTGTGAAC AGAAGCGTG | 1366 |
| AD-1290124 | gsasagc(Ghd)UfgCfAf Ufgagugggcsasa | 767 | VPusUfsgccCfaCfUfcau gCfaCfgcuucsusg | 1067 | CAGAAGCGTGCAT GAGTGGGCAG | 1367 |
| AD-1290125 | csasccu(Ahd)CfgUfAf Ufuuaagaugsasa | 768 | VPusUfscauCfuUfAfaau aCfgUfaggugsgsc | 1068 | GCCACCTACGTATT TAAGATGAA | 1368 |
| AD-1290126 | usasaga(Uhd)GfaAfGf Gfauggagugscsa | 769 | VPusGfscacUfcCfAfucc uUfcAfucuuasasa | 1069 | TTTAAGATGAAGGA TGGAGTGCC | 1369 |
| AD-1290127 | csusgau(Chd)AfuUfG fCfagccggagsgsa | 770 | VPusCfscucCfgGfCfugc aAfuGfaucagsgsg | 1070 | CCCTGATCATTGCA GCCGGAGGT | 1370 |
| AD-1290128 | cscsaag(Ahd)CfaGfAf Cfacguuccascsa | 771 | VPusGfsuggAfaCfGfugu cUfgUfcuuggscsc | 1071 | GGCCAAGACAGAC ACGTTCCACC | 1371 |
| AD-1290129 | asgsaga(Ghd)AfcUfGf Gfagaauaacsusa | 772 | VPusAfsguuAfuUfCfucc aGfuCfucucus gsg | 1072 | CCAGAGAGACTGG AGAATAACTC | 1372 |
| AD-1290130 | gsasaua(Ahd)CfuCfCf Ufcgguucuasgsa | 773 | VPusCfsuagAfaCfCfgag gAfgUfuauucsusc | 1073 | GAGAATAACTCCTC GGTTCTAGG | 1373 |
| AD-1290131 | asgsggc(Uhd)AfaAfCf Gfgcaauuccsgsa | 774 | VPusCfsggaAfuUfGfccg uUfuAfgcccusasg | 1074 | CTAGGGCTAAACG GCAATTCCGG | 1374 |
| AD-1290132 | asusucc(Ghd)GfaGfCf Cfgcagguggsusa | 775 | VPusAfsccaCfcUfGfcgg cUfcCfggaaususg | 1075 | CAATTCCGGAGCCG CAGGTGGTG | 1375 |
| AD-1290133 | gsusggu(Ghd)GfaGfG fUfggcuggaasusa | 776 | VPusAfsuucCfaGfCfcac cUfcCfaccacscsu | 1076 | AGGTGGTGGAGGT GGCTGGAATG | 1376 |
| AD-1290134 | usgsgaa(Uhd)GfaUfA fAfcacuuccususa | 777 | VPusAfsaggAfaGfUfguu aUfcAfuuccasgsc | 1077 | GCTGGAATGATAAC ACTTCCTTG | 1377 |
| AD-1290135 | ascsuuc(Chd)UfuGfCf Ufcugggccgsgsa | 778 | VPusCfscggCfcCfAfgag cAfaGfgaagusgsu | 1078 | ACACTTCCTTGCTC TGGGCCGGA | 1378 |
| AD-1290136 | gsgsccg(Ghd)AfaAfA fUfcuuugcagsgsa | 779 | VPusCfscugCfaAfAfgau uUfuCfcggccscsa | 1079 | TGGGCCGGAAAAT CTTTGCAGGA | 1379 |
| AD-1290137 | usgscag(Ghd)AfgGfG fUfgccaccggsasa | 780 | VPusUfsccgGfuGfGfcac cCfuCfcugcasasa | 1080 | TTTGCAGGAGGGTG CCACCGGAG | 1380 |
| AD-1290138 | cscsacc(Ghd)GfaGfGf Afcauuccugscsa | 781 | VPusGfscagGfaAfUfguc cUfcCfgguggscsa | 1081 | TGCCACCGGAGGA CATTCCTGCC | 1381 |
| AD-1290139 | asgsgcg(Ghd)AfgGfA fUfauauaggcsgsa | 782 | VPusCfsgccUfaUfAfuau cCfuCfcgccuscsc | 1082 | GGAGGCGGAGGAT ATATAGGCGG | 1382 |
| AD-1290140 | asusgca(Ghd)CfcUfCf Afaacaaugascsa | 783 | VPusGfsucaU fuGfUfuug aGfgCfugcaususg | 1083 | CAATGCAGCCTCAA ACAATGACC | 1383 |
| AD-1290141 | gsusuuc(Chd)UfuCfA fUfcaguccacsusa | 784 | VPusAfsgugGfaCfUfgau gAfaGfgaaacscsc | 1084 | GGGTTTCCTTCATC AGTCCACTG | 1384 |
| AD-1290142 | csascug(Ghd)GfcAfUf Cfcuguacacscsa | 785 | VPusGfsgugUfaCfAfgga uGfcCfcagugsgsa | 1085 | TCCACTGGGCATCC TGTACACCC | 1385 |
| AD-1290143 | csasgcu(Uhd)UfaAfAf Afgugauggasasa | 786 | VPusUfsuccAfuCfAfcuu uUfaAfagcugsgsg | 1086 | CCCAGCTTTAAAAG TGATGGAAG | 1386 |
| AD-1290144 | usasagc(Ahd)UfuAfU fCfuaaacugcsasa | 787 | VPusUfsgcaGfuUfUfaga uAfaUfgcuuasasu | 1087 | ATTAAGCATTATCT AAACTGCAG | 1387 |
| AD-1290145 | usgscag(Uhd)CfaCfUf Gfugagguagsasa | 788 | VPusUfscuaCfcUfCfaca gUfgAfcugcasgsu | 1088 | ACTGCAGTCACTGT GAGGTAGAC | 1388 |
| AD-1290146 | gsusaga(Chd)GfaAfUf Gfucacauggsasa | 789 | VPusUfsccaUfgUfGfaca uUfcGfucuacscsu | 1089 | AGGTAGACGAATG TCACATGGAC | 1389 |
| AD-1290147 | cscscug(Ahd)AfaGfCf Cfacaaggucsasa | 790 | VPusUfsgacCfuUfGfugg cUfuUfcagggsusc | 1090 | GACCCTGAAAGCC ACAAGGTCAT | 1390 |
| AD-1290148 | asasggu(Chd)AfuCfUf Gfcuucugugsasa | 791 | VPusUfscacAfgAfAfgca gAfuGfaccuusgsu | 1091 | ACAAGGTCATCTGC TTCTGTGAC | 1391 |
| AD-1290149 | ususcug(Uhd)GfaCfCf Afcgggacggsusa | 792 | VPusAfsccgUfcCfCfgug gUfcAfcagaasgsc | 1092 | GCTTCTGTGACCAC GGGACGGTG | 1392 |
| AD-1290150 | gscsugg(Chd)UfgAfG fGfauggcgucsusa | 793 | VPusAfsgacGfcCfAfucc uCfaGfccagcsasc | 1093 | GTGCTGGCTGAGGA TGGCGTCTC | 1393 |
| AD-1290151 | gsuscuc(Chd)UfgCfAf Ufugugucacscsa | 794 | VPusGfsgugAfcAfCfaau gCfaGfgagacsgsc | 1094 | GCGTCTCCTGCATT GTGTCACCC | 1394 |
| AD-1290152 | cscsaca(Chd)CfuGfCf Cfacucucgcsusa | 795 | VPusAfsgcgAfgAfGfug gcAfgGfuguggscsu | 1095 | AGCCACACCTGCCA CTCTCGCTG | 1395 |
| AD-1290153 | uscsgcu(Ghd)AfuCfCf Ufcucuguggsusa | 796 | VPusAfsccaCfaGfAfgag gAfuCfagcgasgsa | 1096 | TCTCGCTGATCCTC TCTGTGGTG | 1396 |
| AD-1290154 | uscsugu(Ghd)GfuGfA fCfcucugcccsusa | 797 | VPusAfsgggCfaGfAfggu cAfcCfacagasgsa | 1097 | TCTCTGTGGTGACC TCTGCCCTC | 1397 |
| AD-1290155 | gsusccu(Ghd)GfcUfU fUfcuccggcasusa | 798 | VPusAfsugcCfgGfAfgaa aGfcCfaggacscsa | 1098 | TGGTCCTGGCTTTC TCCGGCATC | 1398 |
| AD-1290156 | uscscgg(Chd)AfuCfAf Ufgauugugusasa | 799 | VPusUfsacaCfaAfUfcau gAfuGfccggasgsa | 1099 | TCTCCGGCATCATG ATTGTGTAC | 1399 |
| AD-1290157 | usasccg(Chd)CfgGfAf Afgcaccaggsasa | 800 | VPusUfsccuGfgUfGfcuu cCfgGfcgguascsa | 1100 | TGTACCGCCGGAAG CACCAGGAG | 1400 |
| AD-1290158 | csascca(Ghd)GfaGfCf Ufgcaagccasusa | 801 | VPusAfsuggCfuUfGfcag cUfcCfuggugscsu | 1101 | AGCACCAGGAGCT GCAAGCCATG | 1401 |
| AD-1290159 | csasagc(Chd)AfuGfCf Afgauggagcsusa | 802 | VPusAfsgcuCfcAfUfcug cAfuGfgcuugscsa | 1102 | TGCAAGCCATGCAG ATGGAGCTG | 1402 |
| AD-1290160 | asusgga(Ghd)CfuGfCf Afgagcccugsasa | 803 | VPusUfscagGfgCfUfcug cAfgCfuccauscsu | 1103 | AGATGGAGCTGCA GAGCCCTGAG | 1403 |
| AD-1290161 | cscsuga(Ghd)UfaCfAf Afgcugagcasasa | 804 | VPusUfsugcUfcAfGfcuu gUfaCfucaggsgsc | 1104 | GCCCTGAGTACAAG CTGAGCAAG | 1404 |
| AD-1290162 | gscsucc(Ghd)CfaCfCf Ufcgaccaucsasa | 805 | VPusUfsgauGfgUfCfgag gUfgCfggagcsusu | 1105 | AAGCTCCGCACCTC GACCATCAT | 1405 |
| AD-1290163 | ascscau(Chd)AfuGfAf Cfcgacuacasasa | 806 | VPusUfsuguAfgUfCfgg ucAfuGfaugguscsg | 1106 | CGACCATCATGACC GACTACAAC | 1406 |
| AD-1290164 | csasacu(Ahd)CfuGfCf Ufuugcuggcsasa | 807 | VPusUfsgccAfgCfAfaag cAfgUfaguugsgsg | 1107 | CCCAACTACTGCTT TGCTGGCAA | 1407 |
| AD-1290165 | usgscug(Ghd)CfaAfG fAfccuccuccsasa | 808 | VPusUfsggaGfgAfGfguc uUfgCfcagcasasa | 1108 | TTTGCTGGCAAGAC CTCCTCCAT | 1408 |
| AD-1290166 | uscscuc(Chd)AfuCfAf Gfugaccugasasa | 809 | VPusUfsucaGfgUfCfacu gAfuGfgaggasgsg | 1109 | CCTCCTCCATCAGT GACCTGAAG | 1409 |
| AD-1290167 | usgsaag(Ghd)AfgGfU fGfccgcggaasasa | 810 | VPusUfsuucCfgCfGfgca cCfuCfcuucasgsg | 1110 | CCTGAAGGAGGTG CCGCGGAAAA | 1410 |
| AD-1290168 | gsasggu(Ghd)UfaUfG fAfaggccaggsusa | 811 | VPusAfsccuGfgCfCfuuc aUfaCfaccucscsc | 1111 | GGGAGGTGTATGA AGGCCAGGTG | 1411 |
| AD-1290169 | gsgsugu(Chd)CfgGfA fAfugcccaacsgsa | 812 | VPusCfsguuGfgGfCfauu cCfgGfacaccsusg | 1112 | CAGGTGTCCGGAAT GCCCAACGA | 1412 |
| AD-1290170 | gscsugu(Ghd)AfaGfA fCfgcugccugsasa | 813 | VPusUfscagGfcAfGfcgu cUfuCfacagcscsa | 1113 | TGGCTGTGAAGACG CTGCCTGAA | 1413 |
| AD-1290171 | usgsugc(Uhd)CfuGfA fAfcaggacgasasa | 814 | VPusUfsucgUfcCfUfguu cAfgAfgcacascsu | 1114 | AGTGTGCTCTGAAC AGGACGAAC | 1414 |
| AD-1290172 | gsgsacg(Ahd)AfcUfG fGfauuuccucsasa | 815 | VPusUfsgagGfaAfAfucc aGfuUfcguccsus g | 1115 | CAGGACGAACTGG ATTTCCTCAT | 1415 |
| AD-1290173 | ususccu(Chd)AfuGfG fAfagcccugasusa | 816 | VPusAfsucaGfgGfCfuuc cAfuGfaggaasasu | 1116 | ATTTCCTCATGGAA GCCCTGATC | 1416 |
| AD-1290174 | gsasuca(Uhd)CfaGfCf Afaauucaacscsa | 817 | VPusGfsguuGfaAfUfuu gcUfgAfugaucsasg | 1117 | CTGATCATCAGCAA ATTCAACCA | 1417 |
| AD-1290175 | csasacc(Ahd)CfcAfGf Afacauuguuscsa | 818 | VPusGfsaacAfaUfGfuuc uGfgUfgguugsasa | 1118 | TTCAACCACCAGAA CATTGTTCG | 1418 |
| AD-1290176 | csgsguu(Chd)AfuCfCf Ufgcuggagcsusa | 819 | VPusAfsgcuCfcAfGfcag gAfuGfaaccgsgsg | 1119 | CCCGGTTCATCCTG CTGGAGCTC | 1419 |
| AD-1290177 | asgsacc(Uhd)CfaAfGf Ufccuuccucscsa | 820 | VPusGfsgagGfaAfGfgac uUfgAfggucuscsc | 1120 | GGAGACCTCAAGTC CTTCCTCCG | 1420 |
| AD-1290178 | asusgcu(Ghd)GfaCfCf Ufucugcacgsusa | 821 | VPusAfscguGfcAfGfaag gUfcCfagcausgsg | 1121 | CCATGCTGGACCTT CTGCACGTG | 1421 |
| AD-1290179 | uscsggg(Ahd)CfaUfU fGfccuguggcsusa | 822 | VPusAfsgccAfcAfGfgca aUfgUfcccgasgsc | 1122 | GCTCGGGACATTGC CTGTGGCTG | 1422 |
| AD-1290180 | gsusggc(Uhd)GfuCfA fGfuauuuggasgsa | 823 | VPusCfsuccAfaAfUfacu gAfcAfgccacsasg | 1123 | CTGTGGCTGTCAGT ATTTGGAGG | 1423 |
| AD-1290181 | usgsgag(Ghd)AfaAfA fCfcacuucauscsa | 824 | VPusGfsaugAfaGfUfggu uUfuCfcuccasasa | 1124 | TTTGGAGGAAAACC ACTTCATCC | 1424 |
| AD-1290182 | cscsgag(Ahd)CfaUfUf Gfcugccagasasa | 825 | VPusUfsucuGfgCfAfgca aUfgUfcucggsusg | 1125 | CACCGAGACATTGC TGCCAGAAA | 1425 |
| AD-1290183 | usgscca(Ghd)AfaAfCf Ufgccucuugsasa | 826 | VPusUfscaaGfaGfGfcag uUfuCfuggcasgsc | 1126 | GCTGCCAGAAACTG CCTCTTGAC | 1426 |
| AD-1290184 | cscsucu(Uhd)GfaCfCf Ufguccaggcscsa | 827 | VPusGfsgccUfgGfAfcag gUfcAfagaggscsa | 1127 | TGCCTCTTGACCTG TCCAGGCCC | 1427 |
| AD-1290185 | gscsccu(Ghd)GfaAfGf Afguggccaasgsa | 828 | VPusCfsuugGfcCfAfcuc uUfcCfagggcscsu | 1128 | AGGCCCTGGAAGA GTGGCCAAGA | 1428 |
| AD-1290186 | gsgscca(Ahd)GfaUfUf Gfgagacuucsgsa | 829 | VPusCfsgaaGfuCfUfcca aUfcUfuggccsasc | 1129 | GTGGCCAAGATTGG AGACTTCGG | 1429 |
| AD-1290187 | csusucg(Ghd)GfaUfG fGfcccgagacsasa | 830 | VPusUfsgucUfcGfGfgcc aUfcCfcgaagsusc | 1130 | GACTTCGGGATGGC CCGAGACAT | 1430 |
| AD-1290188 | cscsgag(Ahd)CfaUfCf Ufacagggcgsasa | 831 | VPusUfscgcCfcUfGfuag aUfgUfcucggsgsc | 1131 | GCCCGAGACATCTA CAGGGCGAG | 1431 |
| AD-1290189 | gsasgcu(Ahd)CfuAfU fAfgaaagggasgsa | 832 | VPusCfsuccCfuUfUfcua uAfgUfagcucsgsc | 1132 | GCGAGCTACTATAG AAAGGGAGG | 1432 |
| AD-1290190 | gsgsgag(Ghd)CfuGfU fGfccaugcugscsa | 833 | VPusGfscagCfaUfGfgca cAfgCfcucccsusu | 1133 | AAGGGAGGCTGTG CCATGCTGCC | 1433 |
| AD-1290191 | asusgcu(Ghd)CfcAfGf Ufuaaguggasusa | 834 | VPusAfsuccAfcUfUfaac uGfgCfagcausgsg | 1134 | CCATGCTGCCAGTT AAGTGGATG | 1434 |
| AD-1290192 | gsasggc(Chd)UfuCfAf Ufggaaggaasusa | 835 | VPusAfsuucCfuUfCfcau gAfaGfgccucsusg | 1135 | CAGAGGCCTTCATG GAAGGAATA | 1435 |
| AD-1290193 | gsasagg(Ahd)AfuAfU fUfcacuucuasasa | 836 | VPusUfsuagAfaGfUfgaa uAfuUfccuucscsa | 1136 | TGGAAGGAATATTC ACTTCTAAA | 1436 |
| AD-1290194 | ascsuuc(Uhd)AfaAfAf Cfagacacausgsa | 837 | VPusCfsaugUfgUfCfugu uUfuAfgaagusgsa | 1137 | TCACTTCTAAAACA GACACATGG | 1437 |
| AD-1290195 | ascsaug(Ghd)UfcCfUf Ufuggagugcsusa | 838 | VPusAfsgcaCfuCfCfaaa gGfaCfcaugusgsu | 1138 | ACACATGGTCCTTT GGAGTGCTG | 1438 |
| AD-1290196 | gsusgcu(Ghd)CfuAfU fGfggaaaucususa | 839 | VPusAfsagaUfuUfCfcca uAfgCfagcacsusc | 1139 | GAGTGCTGCTATGG GAAATCTTT | 1439 |
| AD-1290197 | gsasaau(Chd)UfuUfLJf Cfucuuggausasa | 840 | VPusUfsaucCfaAfGfaga aAfaGfauuucscsc | 1140 | GGGAAATCTTTTCT CTTGGATAT | 1440 |
| AD-1290198 | csasaaa(Ghd)CfaAfCf Cfaggaaguuscsa | 841 | VPusGfsaacUfuCfCfugg uUfgCfuuuugscsu | 1141 | AGCAAAAGCAACC AGGAAGTTCT | 1441 |
| AD-1290199 | gsgsaag(Uhd)UfcUfG fGfaguuugucsasa | 842 | VPusUfsgacAfaAfCfucc aGfaAfcuuccsusg | 1142 | CAGGAAGTTCTGGA GTTTGTCAC | 1442 |
| AD-1290200 | gsusuug(Uhd)CfaCfCf Afguggaggcscsa | 843 | VPusGfsgccUfcCfAfcug gUfgAfcaaacsusc | 1143 | GAGTTTGTCACCAG TGGAGGCCG | 1443 |
| AD-1290201 | cscsacc(Chd)AfaGfAf Afcugcccugsgsa | 844 | VPusCfscagGfgCfAfguu cUfuGfgguggsgsu | 1144 | ACCCACCCAAGAA CTGCCCTGGG | 1444 |
| AD-1290202 | usgsccc(Uhd)GfgGfCf Cfuguauaccsgsa | 845 | VPusCfsgguAfuAfCfagg cCfcAfgggcasgsu | 1145 | ACTGCCCTGGGCCT GTATACCGG | 1445 |
| AD-1290203 | gsusaua(Chd)CfgGfAf Ufaaugacucsasa | 846 | VPusUfsgagUfcAfUfuau cCfgGfuauacsasg | 1146 | CTGTATACCGGATA ATGACTCAG | 1446 |
| AD-1290204 | gscsugg(Chd)AfaCfAf Ufcagccugasasa | 847 | VPusUfsucaGfgCfUfgau gUfuGfccagcsasc | 1147 | GTGCTGGCAACATC AGCCTGAAG | 1447 |
| AD-1290205 | asgsccu(Ghd)AfaGfAf Cfaggcccaascsa | 848 | VPusGfsuugGfgCfCfugu cUfuCfaggcusgsa | 1148 | TCAGCCTGAAGACA GGCCCAACT | 1448 |
| AD-1290206 | asascuu(Uhd)GfcCfAf Ufcauuuuggsasa | 849 | VPusUfsccaAfaAfUfgau gGfcAfaaguusgsg | 1149 | CCAACTTTGCCATC ATTTTGGAG | 1449 |
| AD-1290207 | gsgsaga(Ghd)GfaUfU fGfaauacugcsasa | 850 | VPusUfsgcaGfuAfUfuca aUfcCfucuccsasa | 1150 | TTGGAGAGGATTGA ATACTGCAC | 1450 |
| AD-1290208 | ascscca(Ghd)GfaCfCf Cfggauguaasusa | 851 | VPusAfsuuaCfaUfCfcgg gUfcCfugggusgsc | 1151 | GCACCCAGGACCC GGATGTAATC | 1451 |
| AD-1290209 | gsasugu(Ahd)AfuCfA fAfcaccgcuususa | 852 | VPusAfsaagCfgGfUfguu gAfuUfacaucscsg | 1152 | CGGATGTAATCAAC ACCGCTTTG | 1452 |
| AD-1290210 | ascscgc(Uhd)UfuGfCf Cfgauagaausasa | 853 | VPusUfsauuCfuAfUfcgg cAfaAfgcggusgsu | 1153 | ACACCGCTTTGCCG ATAGAATAT | 1453 |
| AD-1290211 | asusaga(Ahd)UfaUfGf Gfuccacuugsusa | 854 | VPusAfscaaGfuGfGfacc aUfaUfucuauscsg | 1154 | CGATAGAATATGGT CCACTTGTG | 1454 |
| AD-1290212 | cscsacu(Uhd)GfuGfGf Afagaggaagsasa | 855 | VPusUfscuuCfcUfCfuuc cAfcAfaguggsasc | 1155 | GTCCACTTGTGGAA GAGGAAGAG | 1455 |
| AD-1290213 | gsgsaag(Ahd)GfaAfA fGfugccugugsasa | 856 | VPusUfscacAfgGfCfacu uUfcUfcuuccsusc | 1156 | GAGGAAGAGAAAG TGCCTGTGAG | 1456 |
| AD-1290214 | usgsagg(Chd)CfcAfAf Gfgacccugasgsa | 857 | VPusCfsucaGfgGfUfccu uGfgGfccucascsa | 1157 | TGTGAGGCCCAAG GACCCTGAGG | 1457 |
| AD-1290215 | cscsucc(Uhd)CfuCfCf Ufggucucucsasa | 858 | VPusUfsgagAfgAfCfcag gAfgAfggaggsasa | 1158 | TTCCTCCTCTCCTG GTCTCTCAA | 1458 |
| AD-1290216 | uscsucu(Chd)AfaCfAf Gfgcaaaacgsgsa | 859 | VPusCfscguUfuUfGfccu gUfuGfagagascsc | 1159 | GGTCTCTCAACAGG CAAAACGGG | 1459 |
| AD-1290217 | uscsugc(Chd)UfaCfCf Afccuccucusgsa | 860 | VPusCfsagaGfgAfGfgug gUfaGfgcagasgsg | 1160 | CCTCTGCCTACCAC CTCCTCTGG | 1460 |
| AD-1290218 | csusccu(Chd)UfgGfCf Afaggcugcasasa | 861 | VPusUfsugcAfgCfCfuug cCfaGfaggagsgsu | 1161 | ACCTCCTCTGGCAA GGCTGCAAA | 1461 |
| AD-1290219 | csusgca(Ahd)AfgAfA fAfcccacagcsusa | 862 | VPusAfsgcuGfuGfGfgu uuCfuUfugcagscsc | 1162 | GGCTGCAAAGAAA CCCACAGCTG | 1462 |
| AD-1290220 | gscsaga(Ghd)AfuCfUf Cfuguucgagsusa | 863 | VPusAfscucGfaAfCfaga gAfuCfucugcsasg | 1163 | CTGCAGAGATCTCT GTTCGAGTC | 1463 |
| AD-1290221 | gsusucg(Ahd)GfuCfC fCfuagagggcscsa | 864 | VPusGfsgccCfuCfUfagg gAfcUfcgaacsasg | 1164 | CTGTTCGAGTCCCT AGAGGGCCG | 1464 |
| AD-1290222 | gsusgaa(Uhd)AfuGfG fCfauucucucsasa | 865 | VPusUfsgagAfgAfAfugc cAfuAfuucacsgsu | 1165 | ACGTGAATATGGCA TTCTCTCAG | 1465 |
| AD-1290223 | csuscuc(Ahd)GfuCfCf Afacccuccususa | 866 | VPusAfsaggAfgGfGfuu ggAfcUfgagagsasa | 1166 | TTCTCTCAGTCCAA CCCTCCTTC | 1466 |
| AD-1290224 | cscsucc(Uhd)UfcGfGf Afguugcacasasa | 867 | VPusUfsuguGfcAfAfcuc cGfaAfggaggsgsu | 1167 | ACCCTCCTTCGGAG TTGCACAAG | 1467 |
| AD-1290225 | cscsacg(Ghd)AfuCfCf Afgaaacaagscsa | 868 | VPusGfscuuGfuUfUfcug gAfuCfcguggsasc | 1168 | GTCCACGGATCCAG AAACAAGCC | 1468 |
| AD-1290226 | csascca(Ghd)CfuUfGf Ufggaacccasasa | 869 | VPusUfsuggGfuUfCfcac aAfgCfuggugsgsg | 1169 | CCCACCAGCTTGTG GAACCCAAC | 1469 |
| AD-1290227 | gsasacc(Chd)AfaCfGf Ufacggcuccsusa | 870 | VPusAfsggaGfcCfGfuac gUfuGfgguucscsa | 1170 | TGGAACCCAACGTA CGGCTCCTG | 1470 |
| AD-1290228 | uscscug(Ghd)UfuUfA fCfagagaaacscsa | 871 | VPusGfsguuUfcUfCfugu aAfaCfcaggasgsc | 1171 | GCTCCTGGTTTACA GAGAAACCC | 1471 |
| AD-1290229 | usasauc(Chd)UfaUfAf Gfcaaagaagsgsa | 872 | VPusCfscuuCfuUfUfgcu aUfaGfgauuasusu | 1172 | AATAATCCTATAGC AAAGAAGGA | 1472 |
| AD-1290230 | asgsaag(Ghd)AfgCfCf Afcacgacagsgsa | 873 | VPusCfscugUfcGfUfgug gCfuCfcuucususu | 1173 | AAAGAAGGAGCCA CACGACAGGG | 1473 |
| AD-1290231 | asgsgga(Ahd)GfcUfG fUfacugucccsasa | 874 | VPusUfsgggAfcAfGfuac aGfcUfucccuscsc | 1174 | GGAGGGAAGCTGT ACTGTCCCAC | 1474 |
| AD-1290232 | cscsacc(Uhd)AfaCfGf Ufugcaacugsgsa | 875 | VPusCfscagUfuGfCfaac gUfuAfgguggsgsa | 1175 | TCCCACCTAACGTT GCAACTGGG | 1475 |
| AD-1290233 | ascsugc(Uhd)CfcUfAf Gfagcccucususa | 876 | VPusAfsagaGfgGfCfucu aGfgAfgcagusgsa | 1176 | TCACTGCTCCTAGA GCCCTCTTC | 1476 |
| AD-1290234 | uscsgcu(Ghd)AfcUfG fCfcaauaugasasa | 877 | VPusUfsucaUfaUfUfggc aGfuCfagcgasasg | 1177 | CTTCGCTGACTGCC AATATGAAG | 1477 |
| AD-1290235 | asasuau(Ghd)AfaGfGf Afgguaccucsusa | 878 | VPusAfsgagGfuAfCfcuc cUfuCfauauus gsg | 1178 | CCAATATGAAGGA GGTACCTCTG | 1478 |
| AD-1290236 | gsusacc(Uhd)CfuGfUf Ufcaggcuacsgsa | 879 | VPusCfsguaGfcCfUfgaa cAfgAfgguacscsu | 1179 | AGGTACCTCTGTTC AGGCTACGT | 1479 |
| AD-1290237 | gscsuac(Ghd)UfcAfCf Ufucccuugusgsa | 880 | VPusCfsacaAfgGfGfaag uGfaCfguagcscsu | 1180 | AGGCTACGTCACTT CCCTTGTGG | 1480 |
| AD-1290238 | ususgug(Ghd)GfaAfU fGfucaauuacsgsa | 881 | VPusCfsguaAfuUfGfaca uUfcCfcacaasgsg | 1181 | CCTTGTGGGAATGT CAATTACGG | 1481 |
| AD-1290239 | gsgscua(Chd)CfaGfCf Afacagggcususa | 882 | VPusAfsagcCfcUfGfuug cUfgGfuagccsgsu | 1182 | ACGGCTACCAGCA ACAGGGCTTG | 1482 |
| AD-1290240 | ususgcc(Chd)UfuAfG fAfagccgcuascsa | 883 | VPusGfsuagCfgGfCfuuc uAfaGfggcaasgsc | 1183 | GCTTGCCCTTAGAA GCCGCTACT | 1483 |
| AD-1290241 | usgsgag(Chd)UfgGfU fCfauuacgagsgsa | 884 | VPusCfscucGfuAfAfuga cCfaGfcuccasgsg | 1184 | CCTGGAGCTGGTCA TTACGAGGA | 1484 |
| AD-1290242 | ususacg(Ahd)GfgAfU fAfccauucugsasa | 885 | VPusUfscagAfaUfGfgua uCfcUfcguaasusg | 1185 | CATTACGAGGATAC CATTCTGAA | 1485 |
| AD-1290243 | csasuuc(Uhd)GfaAfAf Afgcaagaausasa | 886 | VPusUfsauuCfuUfGfcuu uUfcAfgaaugsgsu | 1186 | ACCATTCTGAAAAG CAAGAATAG | 1486 |
| AD-1290244 | asgsaau(Ahd)GfcAfUf Gfaaccagccsusa | 887 | VPusAfsggcUfgGfUfuca uGfcUfauucususg | 1187 | CAAGAATAGCATG AACCAGCCTG | 1487 |
| AD-1290245 | gsasgcu(Chd)GfgUfCf Gfcacacucascsa | 888 | VPusGfsugaGfuGfUfgcg aCfcGfagcucsasg | 1188 | CTGAGCTCGGTCGC ACACTCACT | 1488 |
| AD-1290246 | ascsacu(Chd)AfcUfLTf Cfucuuccuusgsa | 889 | VPusCfsaagGfaAfGfaga aGfuGfagugusgsc | 1189 | GCACACTCACTTCT CTTCCTTGG | 1489 |
| AD-1290247 | gsgsgau(Chd)CfcUfAf Afgaccguggsasa | 890 | VPusUfsccaCfgGfUfcuu aGfgGfaucccsasa | 1190 | TTGGGATCCCTAAG ACCGTGGAG | 1490 |
| AD-1290248 | ascscgu(Ghd)GfaGfGf Afgagagaggscsa | 891 | VPusGfsccuCfuCfUfcuc cUfcCfacgguscsu | 1191 | AGACCGTGGAGGA GAGAGAGGCA | 1491 |
| AD-1290249 | asgsaga(Ghd)GfcAfAf Ufggcuccuuscsa | 892 | VPusGfsaagGfaGfCfcau uGfcCfucucuscsu | 1192 | AGAGAGAGGCAAT GGCTCCTTCA | 1492 |
| AD-1290250 | gscsucc(Uhd)UfcAfCf Afaaccagagsasa | 893 | VPusUfscucUfgGfUfuug uGfaAfggagcscsa | 1193 | TGGCTCCTTCACAA ACCAGAGAC | 1493 |
| AD-1290251 | gsasgac(Chd)AfaAfUf Gfucacguuususa | 894 | VPusAfsaaaCfgUfGfaca uUfuGfgucucsusg | 1194 | CAGAGACCAAATG TCACGTTTTG | 1494 |
| AD-1290252 | csascgu(Uhd)UfuGfLJ fUfuugugccasasa | 895 | VPusUfsuggCfaCfAfaaa cAfaAfacgugsasc | 1195 | GTCACGTTTTGTTT TGTGCCAAC | 1495 |
| AD-1290253 | gscscaa(Chd)CfuAfUf Ufuugaaguascsa | 896 | VPusGfsuacUfuCfAfaaa uAfgGfuuggcsasc | 1196 | GTGCCAACCTATTT TGAAGTACC | 1496 |
| AD-1290254 | usgsuau(Uhd)UfuGfA fAfaaugcuuusasa | 897 | VPusUfsaaaGfcAfUfuuu cAfaAfauacasgsc | 1197 | GCTGTATTTTGAAA ATGCTTTAG | 1497 |
| AD-1290255 | ususuag(Ahd)AfaGfG fUfuuugagcasusa | 898 | VPusAfsugcUfcAfAfaac cUfuUfcuaaasgsc | 1198 | GCTTTAGAAAGGTT TTGAGCATG | 1498 |
| AD-1290256 | ususgag(Chd)AfuGfG fGfuucauccusasa | 899 | VPusUfsaggAfuGfAfacc cAfuGfcucaasasa | 1199 | TTTTGAGCATGGGT TCATCCTAT | 1499 |
| AD-1290257 | csasucc(Uhd)AfuUfCf Ufuucgaaagsasa | 900 | VPusUfscuuUfcGfAfaag aAfuAfggaugsasa | 1200 | TTCATCCTATTCTTT CGAAAGAA | 1500 |
| AD-1290258 | asasuga(Ghd)UfgAfU fAfaauacaagsgsa | 901 | VPusCfscuuGfuAfUfuua uCfaCfucauususu | 1201 | AAAATGAGTGATA AATACAAGGC | 1501 |
| AD-1290259 | asusaca(Ahd)GfgCfCf Cfagauguggsusa | 902 | VPusAfsccaCfaUfCfugg gCfcUfuguaususu | 1202 | AAATACAAGGCCC AGATGTGGTT | 1502 |
| AD-1290260 | gsasugu(Ghd)GfuUfG fCfauaagguususa | 903 | VPusAfsaacCfuUfAfugc aAfcCfacaucsusg | 1203 | CAGATGTGGTTGCA TAAGGTTTT | 1503 |
| AD-1290261 | usgscau(Ghd)UfuUfG fUfuguauacususa | 904 | VPusAfsaguAfuAfCfaac aAfaCfaugcasusa | 1204 | TATGCATGTTTGTT GTATACTTC | 1504 |
| AD-1290262 | ususccu(Uhd)AfuGfC fLTfucuuucaasasa | 905 | VPusUfsuugAfaAfGfaag cAfuAfaggaas gsu | 1205 | ACTTCCTTATGCTT CTTTCAAAT | 1505 |
| AD-1290263 | ususuca(Ahd)AfuUfG fUfgugugcucsusa | 906 | VPusAfsgagCfaCfAfcac aAfuUfugaaasgsa | 1206 | TCTTTCAAATTGTG TGTGCTCTG | 1506 |
| AD-1290264 | csusgcu(Uhd)CfaAfUf Gfuagucagasasa | 907 | VPusUfsucuGfaCfLTfaca uUfgAfagcagsasg | 1207 | CTCTGCTTCAATGT AGTCAGAAT | 1507 |
| AD-1290265 | asgsuca(Ghd)AfaUfLTf Afgcugcuucsusa | 908 | VPusAfsgaaGfcAfGfcua aUfuCfugacusasc | 1208 | GTAGTCAGAATTAG CTGCTTCTA | 1508 |
| AD-1290266 | usgscuu(Chd)UfaUfG fLTfuucauagususa | 909 | VPusAfsacuAfuGfAfaac aUfaGfaagcasgsc | 1209 | GCTGCTTCTATGTT TCATAGTTG | 1509 |
| AD-1290267 | asusguu(Uhd)CfcUfU fGfccuuguugsasa | 910 | VPusUfscaaCfaAfGfgca aGfgAfaacauscsu | 1210 | AGATGTTTCCTTGC CTTGTTGAT | 1510 |
| AD-1290268 | usgsugg(Ahd)CfaUfG fAfgccauuugsasa | 911 | VPusUfscaaAfuGfGfcuc aUfgUfccacasusc | 1211 | GATGTGGACATGA GCCATTTGAG | 1511 |
| AD-1290269 | ascsgga(Ahd)AfuAfA fAfggaguuaususa | 912 | VPusAfsauaAfcUfCfcuu uAfuUfuccgususc | 1212 | GAACGGAAATAAA GGAGTTATTT | 1512 |
| AD-1290270 | asgsuua(Uhd)UfuGftJ fAfaugacuaasasa | 913 | VPusUfsuuaGfuCfAfuua cAfaAfuaacuscsc | 1213 | GGAGTTATTTGTAA TGACTAAAA | 1513 |

TABLE 4

Modified Sense and Antisense Strand Sequences of Human ALK dsRNA Agents

| Duplex ID | Sense Sequence 5′ to 3′ | SEQ ID NO: | Antisense Sequence 5′ to 3′ | SEQ ID NO: | mRNA Target Sequence 5′ to 3′ | SEQ ID NO: |
|---|---|---|---|---|---|---|
| AD-1334980 | gscsagauGfcGfAfUfc cagcggcsusa | 1514 | VPusAfsgccGfcUfGfgau cGfcAfucugcscsu | 914 | AGGCAGATGCGAT CCAGCGGCTC | 1214 |
| AD-1334981 | csgsguggUfaGfCfAfg cugguacscsa | 1515 | VPusGfsguaCfcAfGfcug cUfaCfcaccgscsu | 915 | AGCGGTGGTAGCA GCTGGTACCT | 1215 |
| AD-1334982 | gscsgcugAfuGfAfUfg ggugaggsasa | 1516 | VPusUfsccuCfaCfCfcau cAfuCfagcgcscsc | 916 | GGGCGCTGATGATG GGTGAGGAG | 1216 |
| AD-1334983 | usgsccugCfgAfAfCfu cugaggasgsa | 1517 | VPusCfsuccUfcAfGfagu uCfgCfaggcascsu | 917 | AGTGCCTGCGAACT CTGAGGAGC | 1217 |
| AD-1334984 | gsgsacgcUfgCfAfAfa cuugcgcsasa | 1518 | VPusUfsgcgCfaAfGfuuu gCfaGfcguccsusu | 918 | AAGGACGCTGCAA ACTTGCGCAG | 1218 |
| AD-1334985 | gscsugggAfuUfCfAfc gcccagasasa | 1519 | VPusUfsucuGfgGfCfgug aAfuCfccagcscsc | 919 | GGGCTGGGATTCAC GCCCAGAAG | 1219 |
| AD-1334986 | gscsccagAfaGfUfUfca gcaggcsasa | 1520 | VPusUfsgccUfgCfUfgaa cUfuCfugggcsgsu | 920 | ACGCCCAGAAGTTC AGCAGGCAG | 1220 |
| AD-1334987 | gscsagacAfgUfCfCfga agccuuscsa | 1521 | VPusGfsaagGfcUfUfcgg aCfuGfucugcscsu | 921 | AGGCAGACAGTCC GAAGCCTTCC | 1221 |
| AD-1334988 | csasgcggAfgAfGfAfu agcuugasgsa | 1522 | VPusCfsucaAfgCfUfauc uCfuCfcgcugscsg | 922 | CGCAGCGGAGAGA TAGCTTGAGG | 1222 |
| AD-1334989 | usgsagggUfgCfGfCfa agacggcsasa | 1523 | VPusUfsgccGfuCfUfugc gCfaCfccucasasg | 923 | CTTGAGGGTGCGCA AGACGGCAG | 1223 |
| AD-1334990 | gsgsgcagAfaGfAfGfc uuggaggsasa | 1524 | VPusUfsccuCfcAfAfgcu cUfuCfugcccsgsg | 924 | CCGGGCAGAAGAG CTTGGAGGAG | 1224 |
| AD-1334991 | gsasgccaAfaAfGfGfaa cgcaaasasa | 1525 | VPusUfsuuuGfcGfUfucc uUfuUfggcucscsu | 925 | AGGAGCCAAAAGG AACGCAAAAG | 1225 |
| AD-1334992 | asasaggcGfgCfCfAfg gacagcgsusa | 1526 | VPusAfscgcUfgUfCfcug gCfcGfccuuususg | 926 | CAAAAGGCGGCCA GGACAGCGTG | 1226 |
| AD-1334993 | cscsgccgUfuCfUfCfag ccuuaasasa | 1527 | VPusUfsuuaAfgGfCfuga gAfaCfggcggscsu | 927 | AGCCGCCGTTCTCA GCCTTAAAA | 1227 |
| AD-1334994 | cscsuuaaAfaGfUfUfg cagagaususa | 1528 | VPusAfsaucUfcUfGfcaa cUfuUfuaaggscsu | 928 | AGCCTTAAAAGTTG CAGAGATTG | 1228 |
| AD-1334995 | gsascgguAfcCfCfAfac ugccacscsa | 1529 | VPusGfsgugGfcAfGfuu ggGfuAfccgucscsu | 929 | AGGACGGTACCCA ACTGCCACCT | 1229 |
| AD-1334996 | csusgccaCfcUfCfCfcu ucaaccsasa | 1530 | VPusUfsgguUfgAfAfgg gaGfgUfggcagsusu | 930 | AACTGCCACCTCCC TTCAACCAT | 1230 |
| AD-1334997 | uscsaaccAfuAfGfUfa guuccucsusa | 1531 | VPusAfsgagGfaAfCfuac uAfuGfguugasasg | 931 | CTTCAACCATAGTA GTTCCTCTG | 1231 |
| AD-1334998 | gsusuccuCfuGfUfAfc cgagcgcsasa | 1532 | VPusUfsgcgCfuCfGfgua cAfgAfggaacsusa | 932 | TAGTTCCTCTGTAC CGAGCGCAG | 1232 |
| AD-1334999 | csgsagcgCfaGfCfGfag cuacagsasa | 1533 | VPusUfscugUfaGfCfucg cUfgCfgcucgsgsu | 933 | ACCGAGCGCAGCG AGCTACAGAC | 1233 |
| AD-1335000 | gsgscucaAfgGfUfCfc cagccagsusa | 1534 | VPusAfscugGfcUfGfgga cCfuUfgagccsusc | 934 | GAGGCTCAAGGTCC CAGCCAGTG | 1234 |
| AD-1335001 | gscscaguGfaGfCfCfca gugugcsusa | 1535 | VPusAfsgcaCfaCfUfggg cUfcAfcuggcsusg | 935 | CAGCCAGTGAGCCC AGTGTGCTT | 1235 |
| AD-1335002 | asgsugugCfuUfGfAfg ugucucusgsa | 1536 | VPusCfsagaGfaCfAfcuc aAfgCfacacusgsg | 936 | CCAGTGTGCTTGAG TGTCTCTGG | 1236 |
| AD-1335003 | gsgsucugUfuUfCfAfu uuagacuscsa | 1537 | VPusGfsaguCfuAfAfaug aAfaCfagaccsusg | 937 | CAGGTCTGTTTCAT TTAGACTCC | 1237 |
| AD-1335004 | csusgcucGfcCfUfCfcg ugcagususa | 1538 | VPusAfsacuGfcAfCfgga gGfcGfagcagsgsa | 938 | TCCTGCTCGCCTCC GTGCAGTTG | 1238 |
| AD-1335005 | gsasaagcAfaGfAfGfac uugcgcsgsa | 1539 | VPusCfsgcgCfaAfGfucu cUfuGfcuuucscsc | 939 | GGGAAAGCAAGAG ACTTGCGCGC | 1239 |
| AD-1335006 | gscsgcgcAfcGfCfAfca guccucsusa | 1540 | VPusAfsgagGfaCfUfgug cGfuGfcgcgcsasa | 940 | TTGCGCGCACGCAC AGTCCTCTG | 1240 |
| AD-1335007 | uscscucuGfgAfGfAfu cagguggsasa | 1541 | VPusUfsccaCfcUfGfauc uCfcAfgaggascsu | 941 | AGTCCTCTGGAGAT CAGGTGGAA | 1241 |
| AD-1335008 | asgsgagcCfgCfUfGfg guaccaasgsa | 1542 | VPusCfsuugGfuAfCfcca gCfgGfcuccususc | 942 | GAAGGAGCCGCTG GGTACCAAGG | 1242 |
| AD-1335009 | gsusaccaAfgGfAfCfu guucagasgsa | 1543 | VPusCfsucuGfaAfCfagu cCfuUfgguacscsc | 943 | GGGTACCAAGGAC TGTTCAGAGC | 1243 |
| AD-1335010 | uscsagagCfcUfCfUfuc ccaucuscsa | 1544 | VPusGfsagaUfgGfGfaag aGfgCfucugasasc | 944 | GTTCAGAGCCTCTT CCCATCTCG | 1244 |
| AD-1335011 | cscsggagAfgCfAfGfu guaaacgsgsa | 1545 | VPusCfscguUfuAfCfacu gCfuCfuccggsgsc | 945 | GCCCGGAGAGCAG TGTAAACGGC | 1245 |
| AD-1335012 | gsgsgagcCfaUfCfGfg gcuccugsusa | 1546 | VPusAfscagGfaGfCfccg aUfgGfcucccsasu | 946 | ATGGGAGCCATCG GGCTCCTGTG | 1246 |
| AD-1335013 | usgscugcUfuUfCfCfa cggcagcsusa | 1547 | VPusAfsgcuGfcCfGfugg aAfaGfcagcasgsc | 947 | GCTGCTGCTTTCCA CGGCAGCTG | 1247 |
| AD-1335014 | cscsacucAfgCfUfAfcu cgcgccsusa | 1548 | VPusAfsggcGfcGfAfgua gCfuGfaguggscsu | 948 | AGCCACTCAGCTAC TCGCGCCTG | 1248 |
| AD-1335015 | csasgaggAfaGfAfGfu cuggcagsusa | 1549 | VPusAfscugCfcAfGfacu cUfuCfcucugscsa | 949 | TGCAGAGGAAGAG TCTGGCAGTT | 1249 |
| AD-1335016 | csusggcaGfuUfGfAfc uucguggsusa | 1550 | VPusAfsccaCfgAfAfguc aAfcUfgccagsasc | 950 | GTCTGGCAGTTGAC TTCGTGGTG | 1250 |
| AD-1335017 | ususcgugGfuGfCfCfc ucgcucususa | 1551 | VPusAfsagaGfcGfAfggg cAfcCfacgaasgsu | 951 | ACTTCGTGGTGCCC TCGCTCTTC | 1251 |
| AD-1335018 | uscsgcucUfuCfCfGfu gucuacgscsa | 1552 | VPusGfscguAfgAfCfacg gAfaGfagcgasgsg | 952 | CCTCGCTCTTCCGT GTCTACGCC | 1252 |
| AD-1335019 | gsuscuacGfcCfCfGfg gaccuacsusa | 1553 | VPusAfsguaGfgUfCfccg gGfcGfuagacsasc | 953 | GTGTCTACGCCCGG GACCTACTG | 1253 |
| AD-1335020 | gsasccuaCfuGfCfUfgc caccauscsa | 1554 | VPusGfsaugGfuGfGfcag cAfgUfaggucscsc | 954 | GGGACCTACTGCTG CCACCATCC | 1254 |
| AD-1335021 | csuscggaGfcUfGfAfa ggcuggcsasa | 1555 | VPusUfsgccAfgCfCfuuc aGfcUfccgagsgsa | 955 | TCCTCGGAGCTGAA GGCTGGCAG | 1255 |
| AD-1335022 | gscsugucCfaGfGfGfu gcugaagsgsa | 1556 | VPusCfscuuCfaGfCfacc cUfgGfacagcsgsu | 956 | ACGCTGTCCAGGGT GCTGAAGGG | 1256 |
| AD-1335023 | csgsugccAfaGfCfAfg uuggugcsusa | 1557 | VPusAfsgcaCfcAfAfcug cUfuGfgcacgscsc | 957 | GGCGTGCCAAGCA GTTGGTGCTG | 1257 |
| AD-1335024 | ususggugCfuGfGfAfg cugggcgsasa | 1558 | VPusUfscgcCfcAfGfcuc cAfgCfaccaascsu | 958 | AGTTGGTGCTGGAG CTGGGCGAG | 1258 |
| AD-1335025 | gsasggcgAfuCfUfUfg gaggguusgsa | 1559 | VPusCfsaacCfcUfCfcaa gAfuCfgccucscsu | 959 | AGGAGGCGATCTTG GAGGGTTGC | 1259 |
| AD-1335026 | csusgcucCfaGfUfUfca aucucasgsa | 1560 | VPusCfsugaGfaUfUfgaa cUfgGfagcagscsc | 960 | GGCTGCTCCAGTTC AATCTCAGC | 1260 |
| AD-1335027 | csasgcgaGfcUfGfUfu caguuggsusa | 1561 | VPusAfsccaAfcUfGfaac aGfcUfcgcugsasg | 961 | CTCAGCGAGCTGTT CAGTTGGTG | 1261 |
| AD-1335028 | ususggugGfaUfUfCfg ccaaggcsgsa | 1562 | VPusCfsgccUfuGfGfcga aUfcCfaccaascsu | 962 | AGTTGGTGGATTCG CCAAGGCGA | 1262 |
| AD-1335029 | gsgsgcgaCfuGfAfGfg auccgccsusa | 1563 | VPusAfsggcGfgAfUfccu cAfgUfcgcccsusu | 963 | AAGGGCGACTGAG GATCCGCCTG | 1263 |
| AD-1335030 | asusccgcCfuGfAfUfg cccgagasasa | 1564 | VPusUfsucuCfgGfGfcau cAfgGfcggauscsc | 964 | GGATCCGCCTGATG CCCGAGAAG | 1264 |
| AD-1335031 | cscsgagaAfgAfAfGfg cgucggasasa | 1565 | VPusUfsuccGfaCfGfccu uCfuUfcucggsgsc | 965 | GCCCGAGAAGAAG GCGTCGGAAG | 1265 |
| AD-1335032 | gsgsgcagAfgAfGfGfg aaggcugsusa | 1566 | VPusAfscagCfcUfUfccc uCfuCfugcccsasc | 966 | GTGGGCAGAGAGG GAAGGCTGTC | 1266 |
| AD-1335033 | gsgscuguCfcGfCfGfg caauucgscsa | 1567 | VPusGfscgaAfuUfGfccg cGfgAfcagccsusu | 967 | AAGGCTGTCCGCGG CAATTCGCG | 1267 |
| AD-1335034 | cscsuucuCfuUfCfCfag aucuucsgsa | 1568 | VPusCfsgaaGfaUfCfugg aAfgAfgaaggscsg | 968 | CGCCTTCTCTTCCA GATCTTCGG | 1268 |
| AD-1335035 | ascsugguCfaUfAfGfc uccuuggsasa | 1569 | VPusUfsccaAfgGfAfgcu aUfgAfccaguscsc | 969 | GGACTGGTCATAGC TCCTTGGAA | 1269 |
| AD-1335036 | cscsuuggAfaUfCfAfc caacaaascsa | 1570 | VPusGfsuuuGfuUfGfgu gaUfuCfcaaggsasg | 970 | CTCCTTGGAATCAC CAACAAACA | 1270 |
| AD-1335037 | csasaacaUfgCfCfUfuc uccuucsusa | 1571 | VPusAfsgaaGfgAfGfaag gCfaUfguuugsusu | 971 | AACAAACATGCCTT CTCCTTCTC | 1271 |
| AD-1335038 | csusucucCfuGfAfUfu auuuuacsasa | 1572 | VPusUfsguaAfaAfUfaau cAfgGfagaagsgsa | 972 | TCCTTCTCCTGATT ATTTTACAT | 1272 |
| AD-1335039 | ususuuacAfuGfGfAfa ucucaccsusa | 1573 | VPusAfsgguGfaGfAfuuc cAfuGfuaaaasusa | 973 | TATTTTACATGGAA TCTCACCTG | 1273 |
| AD-1335040 | uscsucacCfuGfGfAfu aaugaaasgsa | 1574 | VPusCfsuuuCfaUfUfauc cAfgGfugagasusu | 974 | AATCTCACCTGGAT AATGAAAGA | 1274 |
| AD-1335041 | asusgaaaGfaCfUfCfcu ucccuususa | 1575 | VPusAfsaagGfgAfAfgga gUfcUfuucaususa | 975 | TAATGAAAGACTCC TTCCCTTTC | 1275 |
| AD-1335042 | ususcccuUfuCfCfUfg ucucaucsgsa | 1576 | VPusCfsgauGfaGfAfcag gAfaAfgggaasgsg | 976 | CCTTCCCTTTCCTGT CTCATCGC | 1276 |
| AD-1335043 | csasucgcAfgCfCfGfau auggucsusa | 1577 | VPusAfsgacCfaUfAfucg gCfuGfcgaugsasg | 977 | CTCATCGCAGCCGA TATGGTCTG | 1277 |
| AD-1335044 | gsgsagugCfaGfCfUfu ugacuucscsa | 1578 | VPusGfsgaaGfuCfAfaag cUfgCfacuccsasg | 978 | CTGGAGTGCAGCTT TGACTTCCC | 1278 |
| AD-1335045 | cscsacugCfaUfGfAfcc ucaggasasa | 1579 | VPusUfsuccUfgAfGfguc aUfgCfaguggsasg | 979 | CTCCACTGCATGAC CTCAGGAAC | 1279 |
| AD-1335046 | csasggaaCfcAfGfAfgc ugguccsusa | 1580 | VPusAfsggaCfcAfGfcuc uGfgUfuccugsasg | 980 | CTCAGGAACCAGA GCTGGTCCTG | 1280 |
| AD-1335047 | uscsccagAfuGfGfAfc uugcuggsasa | 1581 | VPusUfsccaGfcAfAfguc cAfuCfugggasgsg | 981 | CCTCCCAGATGGAC TTGCTGGAT | 1281 |
| AD-1335048 | asgsagcgUfuCfUfAfa ggagaugscsa | 1582 | VPusGfscauCfuCfCfuua gAfaCfgcucusgsc | 982 | GCAGAGCGTTCTAA GGAGATGCC | 1282 |
| AD-1335049 | asusgcccAfgAfGfGfc uccuuucsusa | 1583 | VPusAfsgaaAfgGfAfgcc uCfuGfggcauscsu | 983 | AGATGCCCAGAGG CTCCTTTCTC | 1283 |
| AD-1335050 | uscscuuuCfuCfCfUfu cucaacascsa | 1584 | VPusGfsuguUfgAfGfaag gAfgAfaaggasgsc | 984 | GCTCCTTTCTCCTTC TCAACACC | 1284 |
| AD-1335051 | asgscugaCfuCfCfAfag cacaccsasa | 1585 | VPusUfsgguGfuGfCfuu ggAfgUfcagcusgsa | 985 | TCAGCTGACTCCAA GCACACCAT | 1285 |
| AD-1335052 | gscsacacCfaUfCfCfug aguccgsusa | 1586 | VPusAfscggAfcUfCfagg aUfgGfugugcsusu | 986 | AAGCACACCATCCT GAGTCCGTG | 1286 |
| AD-1335053 | gsasguccGfuGfGfAfu gaggagcsasa | 1587 | VPusUfsgcuCfcUfCfauc cAfcGfgacucsasg | 987 | CTGAGTCCGTGGAT GAGGAGCAG | 1287 |
| AD-1335054 | csasgcagUfgAfGfCfac ugcacascsa | 1588 | VPusGfsuguGfcAfGfugc uCfaCfugcugscsu | 988 | AGCAGCAGTGAGC ACTGCACACT | 1288 |
| AD-1335055 | usgscacaCfuGfGfCfcg ucucggsusa | 1589 | VPusAfsccgAfgAfCfggc cAfgUfgugcasgsu | 989 | ACTGCACACTGGCC GTCTCGGTG | 1289 |
| AD-1335056 | uscsggugCfaCfAfGfg caccugcsasa | 1590 | VPusUfsgcaGfgUfGfccu gUfgCfaccgasgsa | 990 | TCTCGGTGCACAGG CACCTGCAG | 1290 |
| AD-1335057 | csuscuggAfaGfGfUfa cauugccscsa | 1591 | VPusGfsggcAfaUfGfuac cUfuCfcagagsgsg | 991 | CCCTCTGGAAGGTA CATTGCCCA | 1291 |
| AD-1335058 | gscsugcaAfgAfGfAfg auccuccsusa | 1592 | VPusAfsggaGfgAfUfcuc uCfuUfgcagcscsu | 992 | AGGCTGCAAGAGA GATCCTCCTG | 1292 |
| AD-1335059 | asusccucCfuGfAfUfg cccacucscsa | 1593 | VPusGfsgagUfgGfGfcau cAfgGfaggauscsu | 993 | AGATCCTCCTGATG CCCACTCCA | 1293 |
| AD-1335060 | csasgggaAfgCfAfUfg guuggacsasa | 1594 | VPusUfsgucCfaAfCfcau gCfuUfcccugsgsa | 994 | TCCAGGGAAGCAT GGTTGGACAG | 1294 |
| AD-1335061 | ususggacAfgUfGfCfu ccagggasasa | 1595 | VPusUfsuccCfuGfGfagc aCfuGfuccaascsc | 995 | GGTTGGACAGTGCT CCAGGGAAG | 1295 |
| AD-1335062 | cscsagggAfaGfAfAfu cgggcguscsa | 1596 | VPusGfsacgCfcCfGfauu cUfuCfccuggsasg | 996 | CTCCAGGGAAGAA TCGGGCGTCC | 1296 |
| AD-1335063 | cscsagacAfaCfCfC’fau uucgagsusa | 1597 | VPusAfscucGfaAfAfugg gUfuGfucuggsasc | 997 | GTCCAGACAACCCA TTTCGAGTG | 1297 |
| AD-1335064 | uscsgaguGfgCfCfCfu ggaauacsasa | 1598 | VPusUfsguaUfuCfCfagg gCfcAfcucgasasa | 998 | TTTCGAGTGGCCCT GGAATACAT | 1298 |
| AD-1335065 | gsgsaauaCfaUfCfUfcc aguggasasa | 1599 | VPusUfsuccAfcUfGfgag aUfgUfauuccsasg | 999 | CTGGAATACATCTC CAGTGGAAA | 1299 |
| AD-1335066 | csasguggAfaAfCfCfg cagcuugsusa | 1600 | VPusAfscaaGfcUfGfcgg uUfuCfcacugsgsa | 1000 | TCCAGTGGAAACCG CAGCTTGTC | 1300 |
| AD-1335067 | csusgcagUfgGfAfCfu ucuuugcscsa | 1601 | VPusGfsgcaAfaGfAfagu cCfaCfugcagsasc | 1001 | GTCTGCAGTGGACT TCTTTGCCC | 1301 |
| AD-1335068 | cscsugaaGfaAfCfUfgc agugaasgsa | 1602 | VPusCfsuucAfcUfGfcag uUfcUfucaggsgsc | 1002 | GCCCTGAAGAACTG CAGTGAAGG | 1302 |
| AD-1335069 | gsgscuccAfaGfAfUfg gcccugcsasa | 1603 | VPusUfsgcaGfgGfCfcau cUfuGfgagccsusg | 1003 | CAGGCTCCAAGATG GCCCTGCAG | 1303 |
| AD-1335070 | csusccuuCfaCfUfUfg uuggaausgsa | 1604 | VPusCfsauuCfcAfAfcaa gUfgAfaggagscsu | 1004 | AGCTCCTTCACTTG TTGGAATGG | 1304 |
| AD-1335071 | ascsagucCfuCfCfAfgc uugggcsasa | 1605 | VPusUfsgccCfaAfGfcug gAfgGfacuguscsc | 1005 | GGACAGTCCTCCAG CTTGGGCAG | 1305 |
| AD-1335072 | csusgugaCfuUfCfCfac caggacsusa | 1606 | VPusAfsgucCfuGfGfugg aAfgUfcacagsgsc | 1006 | GCCTGTGACTTCCA CCAGGACTG | 1306 |
| AD-1335073 | csasggacUfgUfGfCfcc agggagsasa | 1607 | VPusUfscucCfcUfGfggc aCfaGfuccugsgsu | 1007 | ACCAGGACTGTGCC CAGGGAGAA | 1307 |
| AD-1335074 | csasgggaGfaAfGfAfu gagagccsasa | 1608 | VPusUfsggcUfcUfCfauc uUfcUfcccugsgsg | 1008 | CCCAGGGAGAAGA TGAGAGCCAG | 1308 |
| AD-1335075 | asgsccagAfuGfUfGfc cggaaacsusa | 1609 | VPusAfsguuUfcCfGfgca cAfuCfuggcuscsu | 1009 | AGAGCCAGATGTG CCGGAAACTG | 1309 |
| AD-1335076 | csgsgaaaCfuGfCfCfug uggguususa | 1610 | VPusAfsaacCfcAfCfagg cAfgUfuuccgsgsc | 1010 | GCCGGAAACTGCCT GTGGGTTTT | 1310 |
| AD-1335077 | usgscaacUfuUfGfAfa gauggcususa | 1611 | VPusAfsagcCfaUfCfuuc aAfaGfuugcasgsu | 1011 | ACTGCAACTTTGAA GATGGCTTC | 1311 |
| AD-1335078 | gsasuggcUfuCfUfGfu ggcuggascsa | 1612 | VPusGfsuccAfgCfCfaca gAfaGfccaucsusu | 1012 | AAGATGGCTTCTGT GGCTGGACC | 1312 |
| AD-1335079 | ascsccaaGfgCfAfCfac ugucacscsa | 1613 | VPusGfsgugAfcAfGfug ugCfcUfoggguscsc | 1013 | GGACCCAAGGCAC ACTGTCACCC | 1313 |
| AD-1335080 | ascsuccuCfaAfUfGfgc aggucasgsa | 1614 | VPusCfsugaCfcUfGfcca uUfgAfggagusgsu | 1014 | ACACTCCTCAATGG CAGGTCAGG | 1314 |
| AD-1335081 | gsascccuAfaAfGfGfa ugcccggsusa | 1615 | VPusAfsccgGfgCfAfucc uUfuAfgggucscsu | 1015 | AGGACCCTAAAGG ATGCCCGGTT | 1315 |
| AD-1335082 | gscsccggUfuCfCfAfg gaccaccsasa | 1616 | VPusUfsgguGfgUfCfcug gAfaCfcgggcsasu | 1016 | ATGCCCGGTTCCAG GACCACCAA | 1316 |
| AD-1335083 | gsasccacCfaAfGfAfcc augcucsusa | 1617 | VPusAfsgagCfaUfGfguc uUfgGfuggucscsu | 1017 | AGGACCACCAAGA CCATGCTCTA | 1317 |
| AD-1335084 | usgscucuAfuUfGfCfu caguaccsasa | 1618 | VPusUfsgguAfcUfGfagc aAfuAfgagcasusg | 1018 | CATGCTCTATTGCT CAGTACCAC | 1318 |
| AD-1335085 | gscsuucuGfaAfAfGfu gcuacagsusa | 1619 | VPusAfscugUfaGfCfacu uUfcAfgaagcsgsg | 1019 | CCGCTTCTGAAAGT GCTACAGTG | 1319 |
| AD-1335086 | cscsagugCfuAfCfGfu uuccugcsasa | 1620 | VPusUfsgcaGfgAfAfacg uAfgCfacuggsusc | 1020 | GACCAGTGCTACGT TTCCTGCAC | 1320 |
| AD-1335087 | ascscgauCfaAfGfAfgc ucuccasusa | 1621 | VPusAfsuggAfgAfGfcuc uUfgAfuc ggusgsc | 1021 | GCACCGATCAAGA GCTCTCCATG | 1321 |
| AD-1335088 | csuscuccAfuGfUfGfa gcuccgasasa | 1622 | VPusUfsucgGfaGfCfuca cAfuGfgagagscsu | 1022 | AGCTCTCCATGTGA GCTCCGAAT | 1322 |
| AD-1335089 | gscsuccgAfaUfGfUfc cuggcucsasa | 1623 | VPusUfsgagCfcAfGfgac aUfuCfggagcsusc | 1023 | GAGCTCCGAATGTC CTGGCTCAT | 1323 |
| AD-1335090 | usgsgcucAfuUfCfGfu ggagucususa | 1624 | VPusAfsagaCfuCfCfacg aAfuGfagccasgsg | 1024 | CCTGGCTCATTCGT GGAGTCTTG | 1324 |
| AD-1335091 | gsasaacgUfgUfCfCfu uggugcusasa | 1625 | VPusUfsagcAfcCfAfagg aCfaCfguuucscsc | 1025 | GGGAAACGTGTCCT TGGTGCTAG | 1325 |
| AD-1335092 | gsusgcuaGfuGfGfAfg aacaaaascsa | 1626 | VPusGfsuuuUfgUfUfcuc cAfcUfagcacscsa | 1026 | TGGTGCTAGTGGAG AACAAAACC | 1326 |
| AD-1335093 | gsgsgaagGfaGfCfAfa ggcaggasusa | 1627 | VPusAfsuccUfgCfCfuug cUfcCfuucccsgsg | 1027 | CCGGGAAGGAGCA AGGCAGGATG | 1327 |
| AD-1335094 | gsgscaggAfuGfGfUfc uggcaugsusa | 1628 | VPusAfscauGfcCfAfgac cAfuCfcugccsusu | 1028 | AAGGCAGGATGGT CTGGCATGTC | 1328 |
| AD-1335095 | csgsccuaUfgAfAfGfg cuugagcscsa | 1629 | VPusGfsgcuCfaAfGfccu uCfaUfaggcgsgsc | 1029 | GCCGCCTATGAAGG CTTGAGCCT | 1329 |
| AD-1335096 | ususgagcCfuGfUfGfg caguggasusa | 1630 | VPusAfsuccAfcUfGfcca cAfgGfcucaasgsc | 1030 | GCTTGAGCCTGTGG CAGTGGATG | 1330 |
| AD-1335097 | csasguggAfuGfGfUfg uugccucsusa | 1631 | VPusAfsgagGfcAfAfcac cAfuCfcacugscsc | 1031 | GGCAGTGGATGGT GTTGCCTCTC | 1331 |
| AD-1335098 | csuscgauGfuGfUfCfu gacaggususa | 1632 | VPusAfsaccUfgUfCfaga cAfcAfucgagsgsa | 1032 | TCCTCGATGTGTCT GACAGGTTC | 1332 |
| AD-1335099 | csusggcuGfcAfGfAfu ggucgcasusa | 1633 | VPusAfsugcGfaCfCfauc uGfcAfgccagsasa | 1033 | TTCTGGCTGCAGAT GGTCGCATG | 1333 |
| AD-1335100 | asusccagAfgCfCfAfuc guggcususa | 1634 | VPusAfsagcCfaCfGfaug gCfuCfuggauscsc | 1034 | GGATCCAGAGCCAT CGTGGCTTT | 1334 |
| AD-1335101 | gsusggcuUfuUfGfAfc aauaucuscsa | 1635 | VPusGfsagaUfaUfUfguc aAfaAfgccacsgsa | 1035 | TCGTGGCTTTTGAC AATATCTCC | 1335 |
| AD-1335102 | asasuaucUfcCfAfUfca gccuggsasa | 1636 | VPusUfsccaGfgCfUfgau gGfaGfauauusgsu | 1036 | ACAATATCTCCATC AGCCTGGAC | 1336 |
| AD-1335103 | asgsccugGfaCfUfGfc uaccucascsa | 1637 | VPusGfsugaGfgUfAfgca gUfcCfaggcusgsa | 1037 | TCAGCCTGGACTGC TACCTCACC | 1337 |
| AD-1335104 | usasccucAfcCfAfUfua gcggagsasa | 1638 | VPusUfscucCfgCfUfaau gGfuGfagguasgsc | 1038 | GCTACCTCACCATT AGCGGAGAG | 1338 |
| AD-1335105 | gsgsacaaGfaUfCfCfug cagaausasa | 1639 | VPusUfsauuCfuGfCfagg aUfcUfuguccsusc | 1039 | GAGGACAAGATCC TGCAGAATAC | 1339 |
| AD-1335106 | asgsaauaCfaGfCfAfcc caaaucsasa | 1640 | VPusUfsgauUfuGfGfgu gcUfgUfauucusgsc | 1040 | GCAGAATACAGCA CCCAAATCAA | 1340 |
| AD-1335107 | cscsaaauCfaAfGfAfaa ccuguususa | 1641 | VPusAfsaacAfgGfUfuuc uUfgAfuuuggsgsu | 1041 | ACCCAAATCAAGA AACCTGTTTG | 1341 |
| AD-1335108 | cscsuguuUfgAfGfAfg aaacccasasa | 1642 | VPusUfsuggGfuUfUfcuc uCfaAfacaggsusu | 1042 | AACCTGTTTGAGAG AAACCCAAA | 1342 |
| AD-1335109 | cscscaaaCfaAfGfGfag cugaaascsa | 1643 | VPusGfsuuuCfaGfCfucc uUfgUfuugggsusu | 1043 | AACCCAAACAAGG AGCTGAAACC | 1343 |
| AD-1335110 | gsasaaauUfcAfCfCfaa gacagascsa | 1644 | VPusGfsucuGfuCfUfugg uGfaAfuuuucscsc | 1044 | GGGAAAATTCACC AAGACAGACC | 1344 |
| AD-1335111 | csusuugaCfcCfUfAfca guucaususa | 1645 | VPusAfsaugAfaCfLTfgua gGfgUfcaaagsasu | 1045 | ATCTTTGACCCTAC AGTTCATTG | 1345 |
| AD-1335112 | asgsuucaUfuGfGfCfu guucaccsasa | 1646 | VPusUfsgguGfaAfCfagc cAfaUfgaacusgsu | 1046 | ACAGTTCATTGGCT GTTCACCAC | 1346 |
| AD-1335113 | gscsacagUfgCfAfAfca acgccusasa | 1647 | VPusUfsaggCfgUfUfguu gCfaCfugugcscsu | 1047 | AGGCACAGTGCAA CAACGCCTAC | 1347 |
| AD-1335114 | csusaccaGfaAfCfLTfcc aaccugsasa | 1648 | VPusUfscagGfuUfGfgag uUfcUfgguagsgsc | 1048 | GCCTACCAGAACTC CAACCTGAG | 1348 |
| AD-1335115 | csasaccuGfaGfCfGfug gaggugsgsa | 1649 | VPusCfscacCfuCfCfacg cUfcAfgguugsgsa | 1049 | TCCAACCTGAGCGT GGAGGTGGG | 1349 |
| AD-1335116 | csasuccaGfaUfCfLTfgg aaggugscsa | 1650 | VPusGfscacCfuUfCfcag aUfcUfggaugscsc | 1050 | GGCATCCAGATCTG GAAGGTGCC | 1350 |
| AD-1335117 | gsasagguGfcCfAfGfc caccgacsasa | 1651 | VPusUfsgucGfgUfGfgcu gGfcAfccuucscsa | 1051 | TGGAAGGTGCCAG CCACCGACAC | 1351 |
| AD-1335118 | csasccgaCfaCfCfUfac agcaucsusa | 1652 | VPusAfsgauGfcUfGfuag gUfgUfcggugsgsc | 1052 | GCCACCGACACCTA CAGCATCTC | 1352 |
| AD-1335119 | uscsucggGfcUfAfCfg gagcugcsusa | 1653 | VPusAfsgcaGfcUfCfcgu aGfcCfcgagasusg | 1053 | CATCTCGGGCTACG GAGCTGCTG | 1353 |
| AD-1335120 | gsgscgggAfaAfGfGfc gggaagasasa | 1654 | VPusUfsucuUfcCfCfgcc uUfuCfccgccsasg | 1054 | CTGGCGGGAAAGG CGGGAAGAAC | 1354 |
| AD-1335121 | gsgsgaagAfaCfAfCfca ugaugcsgsa | 1655 | VPusCfsgcaUfcAfUfggu gUfuCfuucccsgsc | 1055 | GCGGGAAGAACAC CATGATGCGG | 1355 |
| AD-1335122 | gsasugcgGfuCfCfCfac ggcgugsusa | 1656 | VPusAfscacGfcCfGfugg gAfcCfgcaucsasu | 1056 | ATGATGCGGTCCCA CGGCGTGTC | 1356 |
| AD-1335123 | gsgscgugUfcUfGfUfg cugggcasusa | 1657 | VPusAfsugcCfcAfGfcac aGfaCfacgccsgsu | 1057 | ACGGCGTGTCTGTG CTGGGCATC | 1357 |
| AD-1335124 | gsgscaucUfuCfAfAfc cuggagasasa | 1658 | VPusUfsucuCfcAfGfguu gAfaGfaugccscsa | 1058 | TGGGCATCTTCAAC CTGGAGAAG | 1358 |
| AD-1335125 | asasggauGfaCfAfUfg cuguacasusa | 1659 | VPusAfsuguAfcAfGfcau gUfcAfuccuuscsu | 1059 | AGAAGGATGACAT GCTGTACATC | 1359 |
| AD-1335126 | csusguacAfuCfCfUfg guugggcsasa | 1660 | VPusUfsgccCfaAfCfcag gAfuGfuacagscsa | 1060 | TGCTGTACATCCTG GTTGGGCAG | 1360 |
| AD-1335127 | gsusugggCfaGfCfAfg ggagaggsasa | 1661 | VPusUfsccuCfuCfCfcug cUfgCfccaacscsa | 1061 | TGGTTGGGCAGCAG GGAGAGGAC | 1361 |
| AD-1335128 | asgsuacaAfaCfCfAfgu uaauccsasa | 1662 | VPusUfsggaUfuAfAfcug gUfuUfguacusgsg | 1062 | CCAGTACAAACCA GTTAATCCAG | 1362 |
| AD-1335129 | ususaaucCfaGfAfAfa gucugcasusa | 1663 | VPusAfsugcAfgAfCfuuu cUfgGfauuaascsu | 1063 | AGTTAATCCAGAAA GTCTGCATT | 1363 |
| AD-1335130 | usgscauuGfgAfGfAfg aacaaugsusa | 1664 | VPusAfscauUfgUfUfcuc uCfcAfaugcasgsa | 1064 | TCTGCATTGGAGAG AACAATGTG | 1364 |
| AD-1335131 | asasugugAfuAfGfAfa gaagaaasusa | 1665 | VPusAfsuuuCfuUfCfuuc uAfuCfacauusgsu | 1065 | ACAATGTGATAGA AGAAGAAATC | 1365 |
| AD-1335132 | asusccguGfuGfAfAfc agaagcgsusa | 1666 | VPusAfscgcUfuCfUfguu cAfcAfcggaususu | 1066 | AAATCCGTGTGAAC AGAAGCGTG | 1366 |
| AD-1335133 | gsasagcgUfgCfAfUfg agugggcsasa | 1667 | VPusUfsgccCfaCfUfcau gCfaCfgcuucsusg | 1067 | CAGAAGCGTGCAT GAGTGGGCAG | 1367 |
| AD-1335134 | csasccuaCfgUfAfUfu uaagaugsasa | 1668 | VPusUfscauCfuUfAfaau aCfgUfaggugsgsc | 1068 | GCCACCTACGTATT TAAGATGAA | 1368 |
| AD-1335135 | usasagauGfaAfGfGfa uggagugscsa | 1669 | VPusGfscacUfcCfAfucc uUfcAfucuuasasa | 1069 | TTTAAGATGAAGGA TGGAGTGCC | 1369 |
| AD-1335136 | csusgaucAfuUfGfCfa gccggagsgsa | 1670 | VPusCfscucCfgGfCfugc aAfuGfaucagsgsg | 1070 | CCCTGATCATTGCA GCCGGAGGT | 1370 |
| AD-1335137 | cscsaagaCfaGfAfCfac guuccascsa | 1671 | VPusGfsuggAfaCfGfugu cUfgUfcuuggscsc | 1071 | GGCCAAGACAGAC ACGTTCCACC | 1371 |
| AD-1335138 | asgsagagAfcUfGfGfa gaauaacsusa | 1672 | VPusAfsguuAfuUfCfucc aGfuCfucucusgsg | 1072 | CCAGAGAGACTGG AGAATAACTC | 1372 |
| AD-1335139 | gsasauaaCfuCfCfLTfcg guucuasgsa | 1673 | VPusCfsuagAfaCfCfgag gAfgUfuauucsusc | 1073 | GAGAATAACTCCTC GGTTCTAGG | 1373 |
| AD-1335140 | asgsggcuAfaAfCfGfg caauuccsgsa | 1674 | VPusCfsggaAfuUfGfccg uUfuAfgcccusasg | 1074 | CTAGGGCTAAACG GCAATTCCGG | 1374 |
| AD-1335141 | asusuccgGfaGfCfCfgc agguggsusa | 1675 | VPusAfsccaCfcUfGfcgg cUfcCfggaaususg | 1075 | CAATTCCGGAGCCG CAGGTGGTG | 1375 |
| AD-1335142 | gsusggugGfaGfGfUfg gcuggaasusa | 1676 | VPusAfsuucCfaGfCfcac cUfcCfaccacscsu | 1076 | AGGTGGTGGAGGT GGCTGGAATG | 1376 |
| AD-1335143 | usgsgaauGfaUfAfAfc acuuccususa | 1677 | VPusAfsaggAfaGfUfguu aUfcAfuuccasgsc | 1077 | GCTGGAATGATAAC ACTTCCTTG | 1377 |
| AD-1335144 | ascsuuccUfuGfCfUfc ugggccgsgsa | 1678 | VPusCfscggCfcCfAfgag cAfaGfgaagusgsu | 1078 | ACACTTCCTTGCTC TGGGCCGGA | 1378 |
| AD-1335145 | gsgsccggAfaAfAfUfc uuugcagsgsa | 1679 | VPusCfscugCfaAfAfgau uUfuCfcggccscsa | 1079 | TGGGCCGGAAAAT CTTTGCAGGA | 1379 |
| AD-1335146 | usgscaggAfgGfGfUfg ccaccggsasa | 1680 | VPusUfsccgGfuGfGfcac cCfuCfcugcasasa | 1080 | TTTGCAGGAGGGTG CCACCGGAG | 1380 |
| AD-1335147 | cscsaccgGfaGfGfAfca uuccugscsa | 1681 | VPusGfscagGfaAfUfguc cUfcCfgguggscsa | 1081 | TGCCACCGGAGGA CATTCCTGCC | 1381 |
| AD-1335148 | asgsgcggAfgGfAfUfa uauaggcsgsa | 1682 | VPusCfsgccUfaUfAfuau cCfuCfcgccuscsc | 1082 | GGAGGCGGAGGAT ATATAGGCGG | 1382 |
| AD-1335149 | asusgcagCfcUfCfAfaa caaugascsa | 1683 | VPusGfsucaUfuGfUfuug aGfgCfugcaususg | 1083 | CAATGCAGCCTCAA ACAATGACC | 1383 |
| AD-1335150 | gsusuuccUfuCfAfUfc aguccacsusa | 1684 | VPusAfsgugGfaCfUfgau gAfaGfgaaacscsc | 1084 | GGGTTTCCTTCATC AGTCCACTG | 1384 |
| AD-1335151 | csascuggGfcAfUfCfc uguacacscsa | 1685 | VPusGfsgugUfaCfAfgga uGfcCfcagugsgsa | 1085 | TCCACTGGGCATCC TGTACACCC | 1385 |
| AD-1335152 | csasgcuuUfaAfAfAfg ugauggasasa | 1686 | VPusUfsuccAfuCfAfcuu uUfaAfagcugsgsg | 1086 | CCCAGCTTTAAAAG TGATGGAAG | 1386 |
| AD-1335153 | usasagcaUfuAfUfCfu aaacugcsasa | 1687 | VPusUfsgcaGfuUfLTfaga uAfaUfgcuuasasu | 1087 | ATTAAGCATTATCT AAACTGCAG | 1387 |
| AD-1335154 | usgscaguCfaCfUfGfu gagguagsasa | 1688 | VPusUfscuaCfcUfCfaca gUfgAfcugcasgsu | 1088 | ACTGCAGTCACTGT GAGGTAGAC | 1388 |
| AD-1335155 | gsusagacGfaAfUfGfu cacauggsasa | 1689 | VPusUfsccaUfgUfGfaca uUfcGfucuacscsu | 1089 | AGGTAGACGAATG TCACATGGAC | 1389 |
| AD-1335156 | cscscugaAfaGfCfCfac aaggucsasa | 1690 | VPusUfsgacCfuUfGfugg cUfuUfcagggsusc | 1090 | GACCCTGAAAGCC ACAAGGTCAT | 1390 |
| AD-1335157 | asasggucAfuCfUfGfc uucugugsasa | 1691 | VPusUfscacAfgAfAfgca gAfuGfaccuusgsu | 1091 | ACAAGGTCATCTGC TTCTGTGAC | 1391 |
| AD-1335158 | ususcuguGfaCfCfAfc gggacggsusa | 1692 | VPusAfsccgUfcCfCfgug gUfcAfcagaasgsc | 1092 | GCTTCTGTGACCAC GGGACGGTG | 1392 |
| AD-1335159 | gscsuggcUfgAfGfGfa uggcgucsusa | 1693 | VPusAfsgacGfcCfAfucc uCfaGfccagcsasc | 1093 | GTGCTGGCTGAGGA TGGCGTCTC | 1393 |
| AD-1335160 | gsuscuccUfgCfAfUfu gugucacscsa | 1694 | VPusGfsgugAfcAfCfaau gCfaGfgagacsgsc | 1094 | GCGTCTCCTGCATT GTGTCACCC | 1394 |
| AD-1335161 | cscsacacCfuGfCfCfac ucucgcsusa | 1695 | VPusAfsgcgAfgAfGfug gcAfgGfuguggscsu | 1095 | AGCCACACCTGCCA CTCTCGCTG | 1395 |
| AD-1335162 | uscsgcugAfuCfCfUfc ucuguggsusa | 1696 | VPusAfsccaCfaGfAfgag gAfuCfagcgasgsa | 1096 | TCTCGCTGATCCTC TCTGTGGTG | 1396 |
| AD-1335163 | uscsugugGfuGfAfCfc ucugcccsusa | 1697 | VPusAfsgggCfaGfAfggu cAfcCfacagasgsa | 1097 | TCTCTGTGGTGACC TCTGCCCTC | 1397 |
| AD-1335164 | gsusccugGfcUfUfUfc uccggcasusa | 1698 | VPusAfsugcCfgGfAfgaa aGfcCfaggacscsa | 1098 | TGGTCCTGGCTTTC TCCGGCATC | 1398 |
| AD-1335165 | uscscggcAfuCfAfUfg auugugusasa | 1699 | VPusUfsacaCfaAfUfcau gAfuGfccggasgsa | 1099 | TCTCCGGCATCATG ATTGTGTAC | 1399 |
| AD-1335166 | usasccgcCfgGfAfAfg caccaggsasa | 1700 | VPusUfsccuGfgUfGfcuu cCfgGfcgguascsa | 1100 | TGTACCGCCGGAAG CACCAGGAG | 1400 |
| AD-1335167 | csasccagGfaGfCfLTfgc aagccasusa | 1701 | VPusAfsuggCfuUfGfcag cUfcCfuggugscsu | 1101 | AGCACCAGGAGCT GCAAGCCATG | 1401 |
| AD-1335168 | csasagccAfuGfCfAfga uggagcsusa | 1702 | VPusAfsgcuCfcAfUfcug cAfuGfgcuugscsa | 1102 | TGCAAGCCATGCAG ATGGAGCTG | 1402 |
| AD-1335169 | asusggagCfuGfCfAfg agcccugsasa | 1703 | VPusUfscagGfgCfUfcug cAfgCfuccauscsu | 1103 | AGATGGAGCTGCA GAGCCCTGAG | 1403 |
| AD-1335170 | cscsugagUfaCfAfAfg cugagcasasa | 1704 | VPusUfsugcUfcAfGfcuu gUfaCfucaggsgsc | 1104 | GCCCTGAGTACAAG CTGAGCAAG | 1404 |
| AD-1335171 | gscsuccgCfaCfCfUfcg accaucsasa | 1705 | VPusUfsgauGfgUfCfgag gUfgCfggagcsusu | 1105 | AAGCTCCGCACCTC GACCATCAT | 1405 |
| AD-1335172 | ascscaucAfuGfAfCfcg acuacasasa | 1706 | VPusUfsuguAfgUfCfgg ucAfuGfaugguscsg | 1106 | CGACCATCATGACC GACTACAAC | 1406 |
| AD-1335173 | csasacuaCfuGfCfUfuu gcuggcsasa | 1707 | VPusUfsgccAfgCfAfaag cAfgUfaguugsgsg | 1107 | CCCAACTACTGCTT TGCTGGCAA | 1407 |
| AD-1335174 | usgscuggCfaAfGfAfc cuccuccsasa | 1708 | VPusUfsggaGfgAfGfguc uUfgCfcagcasasa | 1108 | TTTGCTGGCAAGAC CTCCTCCAT | 1408 |
| AD-1335175 | uscscuccAfuCfAfGfu gaccugasasa | 1709 | VPusUfsucaGfgUfCfacu gAfuGfgaggasgsg | 1109 | CCTCCTCCATCAGT GACCTGAAG | 1409 |
| AD-1335176 | usgsaaggAfgGfUfGfc cgcggaasasa | 1710 | VPusUfsuucCfgCfGfgca cCfuCfcuucasgsg | 1110 | CCTGAAGGAGGTG CCGCGGAAAA | 1410 |
| AD-1335177 | gsasggugUfaUfGfAfa ggccaggsusa | 1711 | VPusAfsccuGfgCfCfuuc aUfaCfaccucscsc | 1111 | GGGAGGTGTATGA AGGCCAGGTG | 1411 |
| AD-1335178 | gsgsugucCfgGfAfAfu gcccaacsgsa | 1712 | VPusCfsguuGfgGfCfauu cCfgGfacaccsusg | 1112 | CAGGTGTCCGGAAT GCCCAACGA | 1412 |
| AD-1335179 | gscsugugAfaGfAfCfg cugccugsasa | 1713 | VPusUfscagGfcAfGfcgu cUfuCfacagcscsa | 1113 | TGGCTGTGAAGACG CTGCCTGAA | 1413 |
| AD-1335180 | usgsugcuCfuGfAfAfc aggacgasasa | 1714 | VPusUfsucgUfcCfUfguu cAfgAfgcacascsu | 1114 | AGTGTGCTCTGAAC AGGACGAAC | 1414 |
| AD-1335181 | gsgsacgaAfcUfGfGfa uuuccucsasa | 1715 | VPusUfsgagGfaAfAfucc aGfuUfcguccsusg | 1115 | CAGGACGAACTGG ATTTCCTCAT | 1415 |
| AD-1335182 | ususccucAfuGfGfAfa gcccugasusa | 1716 | VPusAfsucaGfgGfCfuuc cAfuGfaggaasasu | 1116 | ATTTCCTCATGGAA GCCCTGATC | 1416 |
| AD-1335183 | gsasucauCfaGfCfAfaa uucaacscsa | 1717 | VPusGfsguuGfaAfUfuu gcUfgAfugaucsasg | 1117 | CTGATCATCAGCAA ATTCAACCA | 1417 |
| AD-1335184 | csasaccaCfcAfGfAfac auuguuscsa | 1718 | VPusGfsaacAfaUfGfuuc uGfgUfgguugsasa | 1118 | TTCAACCACCAGAA CATTGTTCG | 1418 |
| AD-1335185 | csgsguucAfuCfCfUfg cuggagcsusa | 1719 | VPusAfsgcuCfcAfGfcag gAfuGfaaccgsgsg | 1119 | CCCGGTTCATCCTG CTGGAGCTC | 1419 |
| AD-1335186 | asgsaccuCfaAfGfUfcc uuccucscsa | 1720 | VPusGfsgagGfaAfGfgac uUfgAfggucuscsc | 1120 | GGAGACCTCAAGTC CTTCCTCCG | 1420 |
| AD-1335187 | asusgcugGfaCfCfUfu cugcacgsusa | 1721 | VPusAfscguGfcAfGfaag gUfcCfagcausgsg | 1121 | CCATGCTGGACCTT CTGCACGTG | 1421 |
| AD-1335188 | uscsgggaCfaUfUfGfc cuguggcsusa | 1722 | VPusAfsgccAfcAfGfgca aUfgUfcccgasgsc | 1122 | GCTCGGGACATTGC CTGTGGCTG | 1422 |
| AD-1335189 | gsusggcuGfuCfAfGfu auuuggasgsa | 1723 | VPusCfsuccAfaAfUfacu gAfcAfgccacsasg | 1123 | CTGTGGCTGTCAGT ATTTGGAGG | 1423 |
| AD-1335190 | usgsgaggAfaAfAfCfc acuucauscsa | 1724 | VPusGfsaugAfaGfUfggu uUfuCfcuccasasa | 1124 | TTTGGAGGAAAACC ACTTCATCC | 1424 |
| AD-1335191 | cscsgagaCfaUfUfGfcu gccagasasa | 1725 | VPusUfsucuGfgCfAfgca aUfgUfcucggsusg | 1125 | CACCGAGACATTGC TGCCAGAAA | 1425 |
| AD-1335192 | usgsccagAfaAfCfUfg ccucuugsasa | 1726 | VPusUfscaaGfaGfGfcag uUfuCfuggcasgsc | 1126 | GCTGCCAGAAACTG CCTCTTGAC | 1426 |
| AD-1335193 | cscsucuuGfaCfCfUfg uccaggcscsa | 1727 | VPusGfsgccUfgGfAfcag gUfcAfagaggscsa | 1127 | TGCCTCTTGACCTG TCCAGGCCC | 1427 |
| AD-1335194 | gscsccugGfaAfGfAfg uggccaasgsa | 1728 | VPusCfsuugGfcCfAfcuc uUfcCfagggcscsu | 1128 | AGGCCCTGGAAGA GTGGCCAAGA | 1428 |
| AD-1335195 | gsgsccaaGfaUfUfGfg agacuucsgsa | 1729 | VPusCfsgaaGfuCfUfcca aUfcUfuggccsasc | 1129 | GTGGCCAAGATTGG AGACTTCGG | 1429 |
| AD-1335196 | csusucggGfaUfGfGfc ccgagacsasa | 1730 | VPusUfsgucUfcGfGfgcc aUfcCfcgaagsusc | 1130 | GACTTCGGGATGGC CCGAGACAT | 1430 |
| AD-1335197 | cscsgagaCfaUfCfUfac agggcgsasa | 1731 | VPusUfscgcCfcUfGfuag aUfgUfcucggsgsc | 1131 | GCCCGAGACATCTA CAGGGCGAG | 1431 |
| AD-1335198 | gsasgcuaCfuAfUfAfg aaag2gasgsa | 1732 | VPusCfsuccCfuUfUfcua uAfgUfagcucsgsc | 1132 | GCGAGCTACTATAG AAAGGGAGG | 1432 |
| AD-1335199 | gsgsgaggCfuGfUfGfc caugcugscsa | 1733 | VPusGfscagCfaUfGfgca cAfgCfcucccsusu | 1133 | AAGGGAGGCTGTG CCATGCTGCC | 1433 |
| AD-1335200 | asusgcugCfcAfGfUfu aaguggasusa | 1734 | VPusAfsuccAfcUfUfaac uGfgCfagcausgsg | 1134 | CCATGCTGCCAGTT AAGTGGATG | 1434 |
| AD-1335201 | gsasggccUfuCfAfUfg gaaggaasusa | 1735 | VPusAfsuucCfuUfCfcau gAfaGfgccucsusg | 1135 | CAGAGGCCTTCATG GAAGGAATA | 1435 |
| AD-1335202 | gsasaggaAfuAfUfUfc acuucuasasa | 1736 | VPusUfsuagAfaGfUfgaa uAfuUfccuucscsa | 1136 | TGGAAGGAATATTC ACTTCTAAA | 1436 |
| AD-1335203 | ascsuucuAfaAfAfCfa gacacausgsa | 1737 | VPusCfsaugUfgUfCfugu uUfuAfgaagusgsa | 1137 | TCACTTCTAAAACA GACACATGG | 1437 |
| AD-1335204 | ascsauggUfcCfUfUfu ggagugcsusa | 1738 | VPusAfsgcaCfuCfCfaaa gGfaCfcaugusgsu | 1138 | ACACATGGTCCTTT GGAGTGCTG | 1438 |
| AD-1335205 | gsusgcugCfuAfUfGfg gaaaucususa | 1739 | VPusAfsagaUfuUfCfcca uAfgCfagcacsusc | 1139 | GAGTGCTGCTATGG GAAATCTTT | 1439 |
| AD-1335206 | gsasaaucUfuUfUfCfu cuuggausasa | 1740 | VPusUfsaucCfaAfGfaga aAfaGfauuucscsc | 1140 | GGGAAATCTTTTCT CTTGGATAT | 1440 |
| AD-1335207 | csasaaagCfaAfCfCfag gaaguuscsa | 1741 | VPusGfsaacUfuCfCfugg uUfgCfuuuugscsu | 1141 | AGCAAAAGCAACC AGGAAGTTCT | 1441 |
| AD-1335208 | gsgsaaguUfcUfGfGfa guuugucsasa | 1742 | VPusUfsgacAfaAfCfucc aGfaAfcuuccsusg | 1142 | CAGGAAGTTCTGGA GTTTGTCAC | 1442 |
| AD-1335209 | gsusuuguCfaCfCfAfg uggaggcscsa | 1743 | VPusGfsgccUfcCfAfcug gUfgAfcaaacsusc | 1143 | GAGTTTGTCACCAG TGGAGGCCG | 1443 |
| AD-1335210 | cscsacccAfaGfAfAfcu gcccugsgsa | 1744 | VPusCfscagGfgCfAfguu cUfuGfgguggsgsu | 1144 | ACCCACCCAAGAA CTGCCCTGGG | 1444 |
| AD-1335211 | usgscccuGfgGfCfCfu guauaccsgsa | 1745 | VPusCfsgguAfuAfCfagg cCfcAfgggcasgsu | 1145 | ACTGCCCTGGGCCT GTATACCGG | 1445 |
| AD-1335212 | gsusauacCfgGfAfUfa augacucsasa | 1746 | VPusUfsgagUfcAfUfuau cCfgGfuauacsasg | 1146 | CTGTATACCGGATA ATGACTCAG | 1446 |
| AD-1335213 | gscsuggcAfaCfAfUfc agccugasasa | 1747 | VPusUfsucaGfgCfUfgau gUfuGfccagcsasc | 1147 | GTGCTGGCAACATC AGCCTGAAG | 1447 |
| AD-1335214 | asgsccugAfaGfAfCfa ggcccaascsa | 1748 | VPusGfsuugGfgCfCfugu cUfuCfaggcusgsa | 1148 | TCAGCCTGAAGACA GGCCCAACT | 1448 |
| AD-1335215 | asascuuuGfcCfAfUfca uuuuggsasa | 1749 | VPusUfsccaAfaAfUfgau gGfcAfaaguusgsg | 1149 | CCAACTTTGCCATC ATTTTGGAG | 1449 |
| AD-1335216 | gsgsagagGfaUfUfGfa auacugcsasa | 1750 | VPusUfsgcaGfuAfUfuca aUfcCfucuccsasa | 1150 | TTGGAGAGGATTGA ATACTGCAC | 1450 |
| AD-1335217 | ascsccagGfaCfCfCfgg auguaasusa | 1751 | VPusAfsuuaCfaUfCfcgg gUfcCfugggusgsc | 1151 | GCACCCAGGACCC GGATGTAATC | 1451 |
| AD-1335218 | gsasuguaAfuCfAfAfc accgcuususa | 1752 | VPusAfsaagCfgGfUfguu gAfuUfacaucscsg | 1152 | CGGATGTAATCAAC ACCGCTTTG | 1452 |
| AD-1335219 | ascscgcuUfuGfCfCfga uagaausasa | 1753 | VPusUfsauuCfuAfUfcgg cAfaAfgcggusgsu | 1153 | ACACCGCTTTGCCG ATAGAATAT | 1453 |
| AD-1335220 | asusagaaUfaUfGfGfu ccacuugsusa | 1754 | VPusAfscaaGfuGfGfacc aUfaUfucuauscsg | 1154 | CGATAGAATATGGT CCACTTGTG | 1454 |
| AD-1335221 | cscsacuuGfuGfGfAfa gaggaagsasa | 1755 | VPusUfscuuCfcUfCfuuc cAfcAfaguggsasc | 1155 | GTCCACTTGTGGAA GAGGAAGAG | 1455 |
| AD-1335222 | gsgsaagaGfaAfAfGfu gccugugsasa | 1756 | VPusUfscacAfgGfCfacu uUfcUfcuuccsusc | 1156 | GAGGAAGAGAAAG TGCCTGTGAG | 1456 |
| AD-1335223 | usgsaggcCfcAfAfGfg acccugasgsa | 1757 | VPusCfsucaGfgGfUfccu uGfgGfccucascsa | 1157 | TGTGAGGCCCAAG GACCCTGAGG | 1457 |
| AD-1335224 | cscsuccuCfuCfCfUfgg ucucucsasa | 1758 | VPusUfsgagAfgAfCfcag gAfgAfggaggsasa | 1158 | TTCCTCCTCTCCTG GTCTCTCAA | 1458 |
| AD-1335225 | uscsucucAfaCfAfGfg caaaacgsgsa | 1759 | VPusCfscguUfuUfGfccu gUfuGfagagascsc | 1159 | GGTCTCTCAACAGG CAAAACGGG | 1459 |
| AD-1335226 | uscsugccUfaCfCfAfcc uccucusgsa | 1760 | VPusCfsagaGfgAfGfgug gUfaGfgcagasgsg | 1160 | CCTCTGCCTACCAC CTCCTCTGG | 1460 |
| AD-1335227 | csusccucUfgGfCfAfa ggcugcasasa | 1761 | VPusUfsugcAfgCfCfuug cCfaGfaggagsgsu | 1161 | ACCTCCTCTGGCAA GGCTGCAAA | 1461 |
| AD-1335228 | csusgcaaAfgAfAfAfc ccacagcsusa | 1762 | VPusAfsgcuGfuGfGfgu uuCfuUfugcagscsc | 1162 | GGCTGCAAAGAAA CCCACAGCTG | 1462 |
| AD-1335229 | gscsagagAfuCfUfCfu guucgagsusa | 1763 | VPusAfscucGfaAfCfaga gAfuCfucugcsasg | 1163 | CTGCAGAGATCTCT GTTCGAGTC | 1463 |
| AD-1335230 | gsusucgaGfuCfCfCfu agagggcscsa | 1764 | VPusGfsgccCfuCfUfagg gAfcUfcgaacsasg | 1164 | CTGTTCGAGTCCCT AGAGGGCCG | 1464 |
| AD-1335231 | gsusgaauAfuGfGfCfa uucucucsasa | 1765 | VPusUfsgagAfgAfAfugc cAfuAfuucacsgsu | 1165 | ACGTGAATATGGCA TTCTCTCAG | 1465 |
| AD-1335232 | csuscucaGfuCfCfAfac ccuccususa | 1766 | VPusAfsaggAfgGfGfuu ggAfcUfgagagsasa | 1166 | TTCTCTCAGTCCAA CCCTCCTTC | 1466 |
| AD-1335233 | cscsuccuUfcGfGfAfg uugcacasasa | 1767 | VPusUfsuguGfcAfAfcuc cGfaAfggaggsgsu | 1167 | ACCCTCCTTCGGAG TTGCACAAG | 1467 |
| AD-1335234 | cscsacggAfuCfCfAfga aacaagscsa | 1768 | VPusGfscuuGfuUfUfcug gAfuCfcguggsasc | 1168 | GTCCACGGATCCAG AAACAAGCC | 1468 |
| AD-1335235 | csasccagCfuUfGfUfg gaacccasasa | 1769 | VPusUfsuggGfuUfCfcac aAfgCfuggugsgsg | 1169 | CCCACCAGCTTGTG GAACCCAAC | 1469 |
| AD-1335236 | gsasacccAfaCfGfUfac ggcuccsusa | 1770 | VPusAfsggaGfcCfGfuac gUfuGfgguucscsa | 1170 | TGGAACCCAACGTA CGGCTCCTG | 1470 |
| AD-1335237 | uscscuggUfuUfAfCfa gagaaacscsa | 1771 | VPusGfsguuUfcUfCfugu aAfaCfcaggasgsc | 1171 | GCTCCTGGTTTACA GAGAAACCC | 1471 |
| AD-1335238 | usasauccUfaUfAfGfca aagaagsgsa | 1772 | VPusCfscuuCfuUfUfgcu aUfaGfgauuasusu | 1172 | AATAATCCTATAGC AAAGAAGGA | 1472 |
| AD-1335239 | asgsaaggAfgCfCfAfca cgacagsgsa | 1773 | VPusCfscugUfcGfUfgug gCfuCfcuucususu | 1173 | AAAGAAGGAGCCA CACGACAGGG | 1473 |
| AD-1335240 | asgsggaaGfcUfGfUfa cugucccsasa | 1774 | VPusUfsgggAfcAfGfuac aGfcUfucccuscsc | 1174 | GGAGGGAAGCTGT ACTGTCCCAC | 1474 |
| AD-1335241 | cscsaccuAfaCfGfUfug caacugsgsa | 1775 | VPusCfscagUfuGfCfaac gUfuAfgguggsgsa | 1175 | TCCCACCTAACGTT GCAACTGGG | 1475 |
| AD-1335242 | ascsugcuCfcUfAfGfa gcccucususa | 1776 | VPusAfsagaGfgGfCfucu aGfgAfgcagusgsa | 1176 | TCACTGCTCCTAGA GCCCTCTTC | 1476 |
| AD-1335243 | uscsgcugAfcUfGfCfc aauaugasasa | 1777 | VPusUfsucaUfaUfUfggc aGfuCfagcgasasg | 1177 | CTTCGCTGACTGCC AATATGAAG | 1477 |
| AD-1335244 | asasuaugAfaGfGfAfg guaccucsusa | 1778 | VPusAfsgagGfuAfCfcuc cUfuCfauauus gsg | 1178 | CCAATATGAAGGA GGTACCTCTG | 1478 |
| AD-1335245 | gsusaccuCfuGfUfUfc aggcuacsgsa | 1779 | VPusCfsguaGfcCfUfgaa cAfgAfgguacscsu | 1179 | AGGTACCTCTGTTC AGGCTACGT | 1479 |
| AD-1335246 | gscsuacgUfcAfCfUfu cccuugusgsa | 1780 | VPusCfsacaAfgGfGfaag uGfaCfguagcscsu | 1180 | AGGCTACGTCACTT CCCTTGTGG | 1480 |
| AD-1335247 | ususguggGfaAfUfGfu caauuacsgsa | 1781 | VPusCfsguaAfuUfGfaca uUfcCfcacaasgsg | 1181 | CCTTGTGGGAATGT CAATTACGG | 1481 |
| AD-1335248 | gsgscuacCfaGfCfAfac agggcususa | 1782 | VPusAfsagcCfcUfGfuug cUfgGfuagccsgsu | 1182 | ACGGCTACCAGCA ACAGGGCTTG | 1482 |
| AD-1335249 | ususgcccUfuAfGfAfa gccgcuascsa | 1783 | VPusGfsuagCfgGfCfuuc uAfaGfggcaasgsc | 1183 | GCTTGCCCTTAGAA GCCGCTACT | 1483 |
| AD-1335250 | usgsgagcUfgGfUfCfa uuacgagsgsa | 1784 | VPusCfscucGfuAfAfuga cCfaGfcuccasgsg | 1184 | CCTGGAGCTGGTCA TTACGAGGA | 1484 |
| AD-1335251 | ususacgaGfgAfUfAfc cauucugsasa | 1785 | VPusUfscagAfaUfGfgua uCfcUfc guaasusg | 1185 | CATTACGAGGATAC CATTCTGAA | 1485 |
| AD-1335252 | csasuucuGfaAfAfAfg caagaausasa | 1786 | VPusUfsauuCfuUfGfcuu uUfcAfgaaugsgsu | 1186 | ACCATTCTGAAAAG CAAGAATAG | 1486 |
| AD-1335253 | asgsaauaGfcAfUfGfaa ccagccsusa | 1787 | VPusAfsggcUfgGfUfuca uGfcUfauucususg | 1187 | CAAGAATAGCATG AACCAGCCTG | 1487 |
| AD-1335254 | gsasgcucGfgUfCfGfc acacucascsa | 1788 | VPusGfsugaGfuGfUfgcg aCfcGfagcucsasg | 1188 | CTGAGCTCGGTCGC ACACTCACT | 1488 |
| AD-1335255 | ascsacucAfcUfUfCfuc uuccuusgsa | 1789 | VPusCfsaagGfaAfGfaga aGfuGfagugusgsc | 1189 | GCACACTCACTTCT CTTCCTTGG | 1489 |
| AD-1335256 | gsgsgaucCfcUfAfAfg accguggsasa | 1790 | VPusUfsccaCfgGfUfcuu aGfgGfaucccsasa | 1190 | TTGGGATCCCTAAG ACCGTGGAG | 1490 |
| AD-1335257 | ascscgugGfaGfGfAfg agagaggscsa | 1791 | VPusGfsccuCfuCfUfcuc cUfcCfacgguscsu | 1191 | AGACCGTGGAGGA GAGAGAGGCA | 1491 |
| AD-1335258 | asgsagagGfcAfAfUfg gcuccuuscsa | 1792 | VPusGfsaagGfaGfCfcau uGfcCfucucuscsu | 1192 | AGAGAGAGGCAAT GGCTCCTTCA | 1492 |
| AD-1335259 | gscsuccuUfcAfCfAfaa ccagagsasa | 1793 | VPusUfscucUfgGfUfuug uGfaAfggagcscsa | 1193 | TGGCTCCTTCACAA ACCAGAGAC | 1493 |
| AD-1335260 | gsasgaccAfaAfUfGfu cacguuususa | 1794 | VPusAfsaaaCfgUfGfaca uUfuGfgucucsusg | 1194 | CAGAGACCAAATG TCACGTTTTG | 1494 |
| AD-1335261 | csascguuUfuGfUfUfu ugugccasasa | 1795 | VPusUfsuggCfaCfAfaaa cAfaAfacgugsasc | 1195 | GTCACGTTTTGTTT TGTGCCAAC | 1495 |
| AD-1335262 | gscscaacCfuAfUfUfu ugaaguascsa | 1796 | VPusGfsuacUfuCfAfaaa uAfgGfuuggcsasc | 1196 | GTGCCAACCTATTT TGAAGTACC | 1496 |
| AD-1335263 | usgsuauuUfuGfAfAfa augcuuusasa | 1797 | VPusUfsaaaGfcAfUfuuu cAfaAfauacasgsc | 1197 | GCTGTATTTTGAAA ATGCTTTAG | 1497 |
| AD-1335264 | ususuagaAfaGfGfUfu uugagcasusa | 1798 | VPusAfsugcUfcAfAfaac cUfuUfcuaaasgsc | 1198 | GCTTTAGAAAGGTT TTGAGCATG | 1498 |
| AD-1335265 | ususgagcAfuGfGfGfu ucauccusasa | 1799 | VPusUfsaggAfuGfAfacc cAfuGfcucaasasa | 1199 | TTTTGAGCATGGGT TCATCCTAT | 1499 |
| AD-1335266 | csasuccuAfuUfCfUfu ucgaaagsasa | 1800 | VPusUfscuuUfcGfAfaag aAfuAfggaugsasa | 1200 | TTCATCCTATTCTTT CGAAAGAA | 1500 |
| AD-1335267 | asasugagUfgAfUfAfa auacaagsgsa | 1801 | VPusCfscuuGfuAfUfuua uCfaCfucauususu | 1201 | AAAATGAGTGATA AATACAAGGC | 1501 |
| AD-1335268 | asusacaaGfgCfCfCfag auguggsusa | 1802 | VPusAfsccaCfaUfCfugg gCfcUfuguaususu | 1202 | AAATACAAGGCCC AGATGTGGTT | 1502 |
| AD-1335269 | gsasugugGfuUfGfCfa uaagguususa | 1803 | VPusAfsaacCfuUfAfugc aAfcCfacaucsusg | 1203 | CAGATGTGGTTGCA TAAGGTTTT | 1503 |
| AD-1335270 | usgscaugUfuUfGfUfu guauacususa | 1804 | VPusAfsaguAfuAfCfaac aAfaCfaugcasusa | 1204 | TATGCATGTTTGTT GTATACTTC | 1504 |
| AD-1335271 | ususccuuAfuGfCfUfu cuuucaasasa | 1805 | VPusUfsuugAfaAfGfaag cAfuAfaggaas gsu | 1205 | ACTTCCTTATGCTT CTTTCAAAT | 1505 |
| AD-1335272 | ususucaaAfuUfGfUfg ugugcucsusa | 1806 | VPusAfsgagCfaCfAfcac aAfuUfugaaasgsa | 1206 | TCTTTCAAATTGTG TGTGCTCTG | 1506 |
| AD-1335273 | csusgcuuCfaAfUfGfu agucagasasa | 1807 | VPusUfsucuGfaCfLTfaca uUfgAfagcagsasg | 1207 | CTCTGCTTCAATGT AGTCAGAAT | 1507 |
| AD-1335274 | asgsucagAfaUfUfAfg cugcuucsusa | 1808 | VPusAfsgaaGfcAfGfcua aUfuCfugacusasc | 1208 | GTAGTCAGAATTAG CTGCTTCTA | 1508 |
| AD-1335275 | usgscuucUfaUfGfUfu ucauagususa | 1809 | VPusAfsacuAfuGfAfaac aUfaGfaagcasgsc | 1209 | GCTGCTTCTATGTT TCATAGTTG | 1509 |
| AD-1335276 | asusguuuCfcUfUfGfc cuuguugsasa | 1810 | VPusUfscaaCfaAfGfgca aGfgAfaacauscsu | 1210 | AGATGTTTCCTTGC CTTGTTGAT | 1510 |
| AD-1335277 | usgsuggaCfaUfGfAfg ccauuugsasa | 1811 | VPusUfscaaAfuGfGfcuc aUfgUfccacasusc | 1211 | GATGTGGACATGA GCCATTTGAG | 1511 |
| AD-1335278 | ascsggaaAfuAfAfAfg gaguuaususa | 1812 | VPusAfsauaAfcUfCfcuu uAfuUfuccgususc | 1212 | GAACGGAAATAAA GGAGTTATTT | 1512 |
| AD-1335279 | asgsuuauUfuGfUfAfa ugacuaasasa | 1813 | VPusUfsuuaGfuCfAfuua cAfaAfuaacuscsc | 1213 | GGAGTTATTTGTAA TGACTAAAA | 1513 |

TABLE 5

ALK Single Dose Screen in Hepal-6 Cells

| Duplex | Avg. % ALK mRNA Remaining | SD |
|---|---|---|
| AD-1335279.1 | 34.525 | 4.720 |
| AD-1335278.1 | 22.249 | 0.409 |
| AD-1335277.1 | 32.476 | 4.544 |
| AD-1335276.1 | 56.501 | 5.568 |
| AD-1335275.1 | 31.774 | 3.685 |
| AD-1335274.1 | 19.568 | 4.475 |
| AD-1335273.1 | 31.890 | 4.348 |
| AD-1335272.1 | 21.263 | 1.699 |
| AD-1335271.1 | 18.237 | 1.269 |
| AD-1335270.1 | 20.560 | 2.704 |
| AD-1335269.1 | 30.105 | 2.374 |
| AD-1335268.1 | 32.149 | 2.442 |
| AD-1335267.1 | 31.028 | 2.349 |
| AD-1335266.1 | 23.572 | 1.382 |
| AD-1335265.1 | 16.734 | 0.611 |
| AD-1335264.1 | 17.114 | 1.640 |
| AD-1335263.1 | 16.698 | 0.657 |
| AD-1335262.1 | 26.554 | 3.578 |
| AD-1335261.1 | 19.377 | 0.396 |
| AD-1335260.1 | 24.884 | 1.448 |
| AD-1335259.1 | 51.189 | 1.962 |
| AD-1335258.1 | 42.896 | 2.328 |
| AD-1335257.1 | 60.622 | 5.298 |
| AD-1335256.1 | 77.018 | 7.335 |
| AD-1335255.1 | 30.180 | 1.041 |
| AD-1335254.1 | 31.972 | 2.405 |
| AD-1335253.1 | 87.537 | 9.487 |
| AD-1335252.1 | 21.777 | 0.559 |
| AD-1335251.1 | 23.041 | 1.134 |
| AD-1335250.1 | 84.774 | 9.186 |
| AD-1335249.1 | 47.047 | 4.090 |
| AD-1335248.1 | 80.658 | 2.896 |
| AD-1335247.1 | 22.825 | 1.648 |
| AD-1335246.1 | 51.903 | 2.850 |
| AD-1335245.1 | 45.131 | 2.604 |
| AD-1335244.1 | 38.811 | 3.290 |
| AD-1335243.1 | 35.207 | 2.216 |
| AD-1335242.1 | 87.361 | 2.464 |
| AD-1335241.1 | 37.048 | 3.688 |
| AD-1335240.1 | 45.067 | 2.015 |
| AD-1335239.1 | 87.389 | 9.637 |
| AD-1335238.1 | 77.232 | 7.178 |
| AD-1335237.1 | 38.172 | 3.901 |
| AD-1335236.1 | 73.725 | 6.294 |
| AD-1335235.1 | 55.126 | 5.373 |
| AD-1335234.1 | 49.370 | 6.123 |
| AD-1335233.1 | 77.590 | 10.436 |
| AD-1335232.1 | 39.098 | 3.947 |
| AD-1335231.1 | 25.141 | 1.697 |
| AD-1335230.1 | 94.084 | 11.532 |
| AD-1335229.1 | 31.738 | 2.946 |
| AD-1335228.1 | 48.532 | 4.698 |
| AD-1335227.1 | 59.271 | 5.278 |
| AD-1335226.1 | 78.110 | 4.756 |
| AD-1335225.1 | 75.155 | 4.824 |
| AD-1335224.1 | 62.445 | 6.995 |
| AD-1335223.1 | 96.380 | 5.346 |
| AD-1335222.1 | 74.637 | 5.192 |
| AD-1335221.1 | 43.910 | 2.561 |
| AD-1335220.1 | 22.630 | 0.692 |
| AD-1335219.1 | 52.349 | 4.878 |
| AD-1335218.1 | 40.687 | 2.393 |
| AD-1335217.1 | 64.859 | 5.312 |
| AD-1335216.1 | 34.433 | 1.556 |
| AD-1335215.1 | 43.378 | 2.362 |
| AD-1335214.1 | 77.320 | 5.527 |
| AD-1335213.1 | 86.509 | 8.177 |
| AD-1335212.1 | 53.743 | 2.403 |
| AD-1335211.1 | 76.516 | 5.246 |
| AD-1335210.1 | 81.981 | 4.388 |
| AD-1335209.1 | 76.061 | 3.333 |
| AD-1335208.1 | 30.934 | 2.901 |
| AD-1335207.1 | 49.666 | 2.564 |
| AD-1335206.1 | 22.026 | 2.309 |
| AD-1335205.1 | 39.954 | 1.020 |
| AD-1335204.1 | 28.261 | 2.488 |
| AD-1335203.1 | 46.953 | 2.970 |
| AD-1335202.1 | 19.822 | 1.283 |
| AD-1335201.1 | 49.168 | 3.484 |
| AD-1335200.1 | 44.070 | 2.162 |
| AD-1335199.1 | 59.688 | 12.610 |
| AD-1335198.1 | 40.256 | 4.244 |
| AD-1335197.1 | 75.243 | 8.557 |
| AD-1335196.1 | 88.524 | 7.266 |
| AD-1335195.1 | 52.810 | 5.052 |
| AD-1335194.1 | 82.952 | 13.041 |
| AD-1335193.1 | 82.342 | 4.474 |
| AD-1335192.1 | 44.186 | 2.273 |
| AD-1335191.1 | 49.934 | 3.235 |
| AD-1335190.1 | 33.144 | 1.596 |
| AD-1335189.1 | 27.127 | 4.517 |
| AD-1335188.1 | 51.094 | 9.355 |
| AD-1335187.1 | 58.621 | 6.308 |
| AD-1335186.1 | 64.407 | 12.630 |
| AD-1335185.1 | 68.800 | 4.242 |
| AD-1335184.1 | 44.100 | 4.218 |
| AD-1335183.1 | 45.608 | 4.432 |
| AD-1335182.1 | 47.879 | 3.431 |
| AD-1335181.1 | 74.027 | 4.740 |
| AD-1335180.1 | 56.176 | 2.939 |
| AD-1335179.1 | 36.742 | 5.251 |
| AD-1335178.1 | 33.293 | 2.541 |
| AD-1335177.1 | 87.986 | 5.783 |
| AD-1335176.1 | 76.270 | 5.869 |
| AD-1335175.1 | 53.384 | 4.071 |
| AD-1335174.1 | 79.133 | 7.740 |
| AD-1335173.1 | 90.962 | 8.945 |
| AD-1335172.1 | 41.617 | 6.575 |
| AD-1335171.1 | 41.976 | 3.836 |
| AD-1335170.1 | 35.361 | 3.696 |
| AD-1335169.1 | 34.037 | 4.310 |
| AD-1335168.1 | 55.503 | 2.742 |
| AD-1335167.1 | 53.536 | 9.242 |
| AD-1335166.1 | 63.783 | 8.871 |
| AD-1335165.1 | 73.401 | 10.844 |
| AD-1335164.1 | 46.341 | 5.060 |
| AD-1335163.1 | 46.566 | 6.156 |
| AD-1335162.1 | 42.831 | 4.682 |
| AD-1335161.1 | 78.571 | 9.575 |
| AD-1335160.1 | 52.058 | 3.381 |
| AD-1335159.1 | 57.938 | 8.717 |
| AD-1335158.1 | 63.829 | 3.835 |
| AD-1335157.1 | 32.051 | 2.907 |
| AD-1335156.1 | 58.638 | 3.339 |
| AD-1335155.1 | 25.275 | 1.745 |
| AD-1335154.1 | 34.570 | 3.616 |
| AD-1335153.1 | 12.723 | 2.092 |
| AD-1335152.1 | 21.822 | 2.947 |
| AD-1335151.1 | 66.557 | 8.248 |
| AD-1335150.1 | 19.584 | 2.830 |
| AD-1335149.1 | 41.383 | 2.489 |
| AD-1335148.1 | 92.171 | 5.187 |
| AD-1335147.1 | 72.294 | 7.192 |
| AD-1335146.1 | 93.303 | 5.528 |
| AD-1335145.1 | 55.502 | 1.749 |
| AD-1335144.1 | 69.150 | 3.271 |
| AD-1335143.1 | 19.955 | 1.872 |
| AD-1335142.1 | 50.998 | 3.009 |
| AD-1335141.1 | 58.642 | 4.654 |
| AD-1335140.1 | 52.077 | 3.465 |
| AD-1335139.1 | 39.423 | 1.824 |
| AD-1335138.1 | 48.093 | 5.073 |
| AD-1335137.1 | 46.471 | 5.936 |
| AD-1335136.1 | 75.214 | 7.956 |
| AD-1335135.1 | 32.103 | 0.513 |
| AD-1335134.1 | 22.128 | 1.335 |
| AD-1335133.1 | 52.382 | 3.319 |
| AD-1335132.1 | 13.408 | 1.130 |
| AD-1335131.1 | 10.810 | 0.689 |
| AD-1335130.1 | 57.551 | 4.566 |
| AD-1335129.1 | 31.502 | 3.642 |
| AD-1335128.1 | 17.407 | 0.821 |
| AD-1335127.1 | 67.904 | 2.683 |
| AD-1335126.1 | 50.473 | 1.955 |
| AD-1335125.1 | 24.809 | 1.602 |
| AD-1335124.1 | 40.486 | 2.293 |
| AD-1335123.1 | 84.745 | 5.320 |
| AD-1335122.1 | 70.516 | 1.881 |
| AD-1335121.1 | 31.007 | 2.014 |
| AD-1335120.1 | 48.339 | 0.741 |
| AD-1335119.1 | 74.531 | 6.548 |
| AD-1335118.1 | 65.332 | 5.598 |
| AD-1335117.1 | 81.616 | 6.733 |
| AD-1335116.1 | 46.230 | 1.045 |
| AD-1335115.1 | 62.360 | 1.700 |
| AD-1335114.1 | 64.549 | 6.270 |
| AD-1335113.1 | 54.666 | 4.713 |
| AD-335112.1 | 43.084 | 4.363 |
| AD-1335111.1 | 49.570 | 6.974 |
| AD-1335110.1 | 48.818 | 4.935 |
| AD-1335109.1 | 50.238 | 5.864 |
| AD-1335108.1 | 49.250 | 0.582 |
| AD-1335107.1 | 51.113 | 3.044 |
| AD-1335106.1 | 63.839 | 4.493 |
| AD-1335105.1 | 59.748 | 1.920 |
| AD-1335104.1 | 78.080 | 7.084 |
| AD-1335103.1 | 76.777 | 3.206 |
| AD-1335102.1 | 69.831 | 6.814 |
| AD-1335101.1 | 46.527 | 4.411 |
| AD-1335100.1 | 68.100 | 4.819 |
| AD-1335099.1 | 48.200 | 6.533 |
| AD-1335098.1 | 68.585 | 2.929 |
| AD-1335097.1 | 60.544 | 5.219 |
| AD-1335096.1 | 65.687 | 3.501 |
| AD-1335095.1 | 91.374 | 4.194 |
| AD-1335094.1 | 76.589 | 4.946 |
| AD-1335093.1 | 96.630 | 6.579 |
| AD-1335092.1 | 49.880 | 5.568 |
| AD-1335091.1 | 58.017 | 3.638 |
| AD-1335090.1 | 58.442 | 5.629 |
| AD-1335089.1 | 64.724 | 2.282 |
| AD-1335088.1 | 59.864 | 1.746 |
| AD-1335087.1 | 62.343 | 2.046 |
| AD-1335086.1 | 68.700 | 1.692 |
| AD-1335085.1 | 45.821 | 1.144 |
| AD-1335084.1 | 48.386 | 2.322 |
| AD-1335083.1 | 53.556 | 4.947 |
| AD-1335082.1 | 80.998 | 2.494 |
| AD-1335081.1 | 65.895 | 3.539 |
| AD-1335080.1 | 86.151 | 8.107 |
| AD-1335079.1 | 72.577 | 1.129 |
| AD-1335078.1 | 69.340 | 4.431 |
| AD-1335077.1 | 41.362 | 1.290 |
| AD-1335076.1 | 75.603 | 4.312 |
| AD-1335075.1 | 80.405 | 4.648 |
| AD-1335074.1 | 72.982 | 2.233 |
| AD-1335073.1 | 90.121 | 2.960 |
| AD-1335072.1 | 87.462 | 3.147 |
| AD-1335071.1 | 87.451 | 4.856 |
| AD-1335070.1 | 62.202 | 6.691 |
| AD-1335069.1 | 89.623 | 4.240 |
| AD-1335068.1 | 85.083 | 8.096 |
| AD-1335067.1 | 46.179 | 3.590 |
| AD-1335066.1 | 83.229 | 3.744 |
| AD-1335065.1 | 47.414 | 3.268 |
| AD-1335064.1 | 76.336 | 5.925 |
| AD-1335063.1 | 52.640 | 2.245 |
| AD-1335062.1 | 76.029 | 6.789 |
| AD-1335061.1 | 80.172 | 3.110 |
| AD-1335060.1 | 76.867 | 4.622 |
| AD-1335059.1 | 74.907 | 1.887 |
| AD-1335058.1 | 63.582 | 4.645 |
| AD-1335057.1 | 69.577 | 7.107 |
| AD-1335056.1 | 89.859 | 7.187 |
| AD-1335055.1 | 71.952 | 7.849 |
| AD-1335054.1 | 87.532 | 7.648 |
| AD-1335053.1 | 98.820 | 11.073 |
| AD-1335052.1 | 76.238 | 9.271 |
| AD-1335051.1 | 98.925 | 11.300 |
| AD-1335050.1 | 52.532 | 4.684 |
| AD-1335049.1 | 75.467 | 9.051 |
| AD-1335048.1 | 74.186 | 4.800 |
| AD-1335047.1 | 64.051 | 4.827 |
| AD-1335046.1 | 106.822 | 10.409 |
| AD-1335045.1 | 56.750 | 2.419 |
| AD-1335044.1 | 44.330 | 1.136 |
| AD-1335043.1 | 55.539 | 2.059 |
| AD-1335042.1 | 61.411 | 1.443 |
| AD-1335041.1 | 68.828 | 3.051 |
| AD-1335040.1 | 31.451 | 0.748 |
| AD-1335039.1 | 21.697 | 0.554 |
| AD-1335038.1 | 27.618 | 1.288 |
| AD-1335037.1 | 38.248 | 1.528 |
| AD-1335036.1 | 45.835 | 2.783 |
| AD-1335035.1 | 46.882 | 1.441 |
| AD-1335034.1 | 64.917 | 3.341 |
| AD-1335033.1 | 102.266 | 5.850 |
| AD-1335032.1 | 86.906 | 5.092 |
| AD-1335031.1 | 84.674 | 1.860 |
| AD-1335030.1 | 90.425 | 3.072 |
| AD-1335029.1 | 99.361 | 3.446 |
| AD-1335028.1 | 79.902 | 4.311 |
| AD-1335027.1 | 68.268 | 2.791 |
| AD-1335026.1 | 41.197 | 1.536 |
| AD-1335025.1 | 67.138 | 2.854 |
| AD-1335024.1 | 93.690 | 3.132 |
| AD-1335023.1 | 70.378 | 6.617 |
| AD-1335022.1 | 86.405 | 3.012 |
| AD-1335021.1 | 99.353 | 2.640 |
| AD-1335020.1 | 87.086 | 7.714 |
| AD-1335019.1 | 75.598 | 4.345 |
| AD-1335018.1 | 62.731 | 3.509 |
| AD-1335017.1 | 62.201 | 2.091 |
| AD-1335016.1 | 47.033 | 0.872 |
| AD-1335015.1 | 61.881 | 3.511 |
| AD-1335014.1 | 57.758 | 2.969 |
| AD-1335013.1 | 99.760 | 5.729 |
| AD-1335012.1 | 110.785 | 2.630 |
| AD-1335011.1 | 90.864 | 9.378 |
| AD-1335010.1 | 62.596 | 3.499 |
| AD-1335009.1 | 50.021 | 2.701 |
| AD-1335008.1 | 107.726 | 4.473 |
| AD-1335007.1 | 57.279 | 3.004 |
| AD-1335006.1 | 65.275 | 2.397 |
| AD-1335005.1 | 73.217 | 2.048 |
| AD-1335004.1 | 92.304 | 2.794 |
| AD-1335003.1 | 31.001 | 2.025 |
| AD-1335002.1 | 68.845 | 5.331 |
| AD-1335001.1 | 67.948 | 7.550 |
| AD-1335000.1 | 93.824 | 7.607 |
| AD-1334999.1 | 74.965 | 6.160 |
| AD-1334998.1 | 53.788 | 4.400 |
| AD-1334997.1 | 30.110 | 1.222 |
| AD-1334996.1 | 79.185 | 4.479 |
| AD-1334995.1 | 93.187 | 2.805 |
| AD-1334994.1 | 36.154 | 1.585 |
| AD-1334993.1 | 61.527 | 4.725 |
| AD-1334992.1 | 93.098 | 8.033 |
| AD-1334991.1 | 51.110 | 4.158 |
| AD-1334990.1 | 77.433 | 6.334 |
| AD-1334989.1 | 88.368 | 7.166 |
| AD-1334988.1 | 48.962 | 3.398 |
| AD-1334987.1 | 63.193 | 2.268 |
| AD-1334986.1 | 60.978 | 4.256 |
| AD-1334985.1 | 69.637 | 2.673 |
| AD-1334984.1 | 79.243 | 6.081 |
| AD-1334983.1 | 83.526 | 5.274 |
| AD-1334982.1 | 55.374 | 3.459 |
| AD-1334981.1 | 87.520 | 2.950 |
| AD-1334980.1 | 73.699 | 5.584 |

### ALK SEQUENCES

SEQ ID NO:1 >NM_004304.4 Homo sapiens ALK receptor tyrosine kinase (ALK), transcript variant 1, mRNA

AGCTGCAAGTGGCGGGCGCCCAGGCAGATGCGATCCAGCGGCTCTGGGGG
CGGCAGCGGTGGTAGCAGCTGGTACCTCCCGCCGCCTCTGTTCGGAGGG
TCGCGGGGCACCGAGGTGCTTTCCGGC CGCCCTCTGGTCGGCCACCCAA
AGCCGCGGGCGCTGATGATGGGTGAGGAGGGGGCGGCAAGATTTCGGGCG
CCCC TGCCCTGAACGCCCTCAGCTGCTGCCGCCGGGGCCGCTCCAGTGC
CTGCGAACTCTGAGGAGCCGAGGCGCCGGTG AGAGCAAGGACGCTGCAA
ACTTGCGCAGCGCGGGGGCTGGGATTCACGCCCAGAAGTTCAGCAGGCAG
ACAGTCCG AAGCCTTCCCGCAGCGGAGAGATAGCTTGAGGGTGCGCAAG
ACGGCAGCCTCCGCCCTCGGTTCCCGCCCAGACCG GGCAGAAGAGCTTG
GAGGAGCCAAAAGGAACGCAAAAGGCGGCCAGGACAGCGTGCAGCAGCTG
GGAGCCGCCGTT CTCAGCCTTAAAAGTTGCAGAGATTGGAGGCTGCCCC
GAGAGGGGACAGACCCCAGCTCCGACTGCGGGGGGCAGG AGAGGACGGT
ACCCAACTGCCACCTCCCTTCAACCATAGTAGTTCCTCTGTACCGAGCGC
AGCGAGCTACAGACGG GGGCGCGGCACTCGGCGCGGAGAGCGGGAGGCT
CAAGGTCCCAGCCAGTGAGCCCAGTGTGCTTGAGTGTCTCTGG ACTCGC
CCCTGAGCTTCCAGGTCTGTTTCATTTAGACTCCTGCTCGCCTCCGTGCA
GTTGGGGGAAAGCAAGAGAC TTGCGCGCACGCACAGTCCTCTGGAGATC
AGGTGGAAGGAGCCGCTGGGTACCAAGGACTGTTCAGAGCCTCTTCC CA
TCTCGGGGAGAGCGAAGGGTGAGGCTGGGCCCGGAGAGCAGTGTAAACGG
CCTCCTCCGGCGGGATGGGAGCCA TCGGGCTCCTGTGGCTCCTGCCGCT
GCTGCTTTCCACGGCAGCTGTGGGCTCCGGGATGGGGACCGGCCAGCGCG
C GGGCTCCCCAGCTGCGGGGCCGCCGCTGCAGCCCCGGGAGCCACTCAG
CTACTCGCGCCTGCAGAGGAAGAGTCTG GCAGTTGACTTCGTGGTGCCC
TCGCTCTTCCGTGTCTACGCCCGGGACCTACTGCTGCCACCATCCTCCTC
GGAGC TGAAGGCTGGCAGGCCCGAGGCCCGCGGCTCGCTAGCTCTGGAC
TGCGCCCCGCTGCTCAGGTTGCTGGGGCCGGC GCCGGGGGTCTCCTGGA
CCGCCGGTTCACCAGCCCCGGCAGAGGCCCGGACGCTGTCCAGGGTGCTG
AAGGGCGGC TCCGTGCGCAAGCTCCGGCGTGCCAAGCAGTTGGTGCTGG
AGCTGGGCGAGGAGGCGATCTTGGAGGGTTGCGTCG GGCCCCCCGGGGA
GGCGGCTGTGGGGCTGCTCCAGTTCAATCTCAGCGAGCTGTTCAGTTGGT
GGATTCGCCAAGG CGAAGGGCGACTGAGGATCCGCCTGATGCCCGAGAA
GAAGGCGTCGGAAGTGGGCAGAGAGGGAAGGCTGTCCGCG GCAATTCGC
GCCTCCCAGCCCCGCCTTCTCTTCCAGATCTTCGGGACTGGTCATAGCTC
CTTGGAATCACCAACAA ACATGCCTTCTCCTTCTCCTGATTATTTTACA
TGGAATCTCACCTGGATAATGAAAGACTCCTTCCCTTTCCTGTC TCATC
GCAGCCGATATGGTCTGGAGTGCAGCTTTGACTTCCCCTGTGAGCTGGAG
TATTCCCCTCCACTGCATGAC CTCAGGAACCAGAGCTGGTCCTGGCGCC
GCATCCCCTCCGAGGAGGCCTCCCAGATGGACTTGCTGGATGGGCCTG G
GGCAGAGCGTTCTAAGGAGATGCCCAGAGGCTCCTTTCTCCTTCTCAACA
CCTCAGCTGACTCCAAGCACACCAT CCTGAGTCCGTGGATGAGGAGCAG
CAGTGAGCACTGCACACTGGCCGTCTCGGTGCACAGGCACCTGCAGCCCT
CT GGAAGGTACATTGCCCAGCTGCTGCCCCACAACGAGGCTGCAAGAGA
GATCCTCCTGATGCCCACTCCAGGGAAGC ATGGTTGGACAGTGCTCCAG
GGAAGAATCGGGCGTCCAGACAACCCATTTCGAGTGGCCCTGGAATACAT
CTCCAG TGGAAACCGCAGCTTGTCTGCAGTGGACTTCTTTGCCCTGAAG
AACTGCAGTGAAGGAACATCCCCAGGCTCCAAG ATGGCCCTGCAGAGCT
CCTTCACTTGTTGGAATGGGACAGTCCTCCAGCTTGGGCAGGCCTGTGAC
TTCCACCAGG ACTGTGCCCAGGGAGAAGATGAGAGCCAGATGTGCCGGA
AACTGCCTGTGGGTTTTTACTGCAACTTTGAAGATGG CTTCTGTGGCTG
GACCCAAGGCACACTGTCACCCCACACTCCTCAATGGCAGGTCAGGACCC
TAAAGGATGCCCGG TTCCAGGACCACCAAGACCATGCTCTATTGCTCAG
TACCACTGATGTCCCCGCTTCTGAAAGTGCTACAGTGACCA GTGCTACG
TTTCCTGCACCGATCAAGAGCTCTCCATGTGAGCTCCGAATGTCCTGGCT
CATTCGTGGAGTCTTGAG GGGAAACGTGTCCTTGGTGCTAGTGGAGAAC
AAAACCGGGAAGGAGCAAGGCAGGATGGTCTGGCATGTCGCCGCC TATG
AAGGCTTGAGCCTGTGGCAGTGGATGGTGTTGCCTCTCCTCGATGTGTCT
GACAGGTTCTGGCTGCAGATGG TCGCATGGTGGGGACAAGGATCCAGAG
CCATCGTGGCTTTTGACAATATCTCCATCAGCCTGGACTGCTACCTCAC
CATTAGCGGAGAGGACAAGATCCTGCAGAATACAGCACCCAAATCAAGAA
ACCTGTTTGAGAGAAACCCAAACAAG GAGCTGAAACCCGGGGAAAATTC
ACCAAGACAGACCCCCATCTTTGACCCTACAGTTCATTGGCTGTTCACCA
CAT GTGGGGCCAGCGGGCCCCATGGCCCCACCCAGGCACAGTGCAACAA
CGCCTACCAGAACTCCAACCTGAGCGTGGA GGTGGGGAGCGAGGGCCCC
CTGAAAGGCATCCAGATCTGGAAGGTGCCAGCCACCGACACCTACAGCAT
CTCGGGC TACGGAGCTGCTGGCGGGAAAGGCGGGAAGAACACCATGATG
CGGTCCCACGGCGTGTCTGTGCTGGGCATCTTCA ACCTGGAGAAGGATG
ACATGCTGTAC ATCCTGGTTGGGCAGCAGGGAGAGGACGCCTGCCCCA
GTACAAACCAGTT AATCCAGAAAGTCTGCATTGGAGAGAACAATGTGAT
AGAAGAAGAAATCCGTGTGAACAGAAGCGTGCATGAGTGG GCAGGAGGC
GGAGGAGGAGGGGGTGGAGCCACCTACGTATTTAAGATGAAGGATGGAGT
GCCGGTGCCCCTGATCA TTGCAGCCGGAGGTGGTGGCAGGGCCTACGGG
GCCAAGACAGACACGTTCCACCCAGAGAGACTGGAGAATAACTC CTCGG
TTCTAGGGCTAAACGGCAATTCCGGAGCCGCAGGTGGTGGAGGTGGCTGG
AATGATAACACTTCCTTGCTC TGGGCCGGAAAATCTTTGCAGGAGGGTG
CCACCGGAGGACATTCCTGCCCCCAGGCCATGAAGAAGTGGGGGTGGG A
GACAAGAGGGGGTTTCGGAGGGGGTGGAGGGGGGTGCTCCTCAGGTGGAG
GAGGCGGAGGATATATAGGCGGCAA TGCAGCCTCAAACAATGACCCCGA
AATGGATGGGGAAGATGGGGTTTCCTTCATCAGTCCACTGGGCATCCTGT
AC ACCCCAGCTTTAAAAGTGATGGAAGGCCACGGGGAAGTGAATATTAA
GCATTATCTAAACTGCAGTCACTGTGAGG TAGACGAATGTCACATGGAC
CCTGAAAGCCACAAGGTCATCTGCTTCTGTGACCACGGGACGGTGCTGGC
TGAGGA TGGCGTCTCCTGCATTGTGTCACCCACCCCGGAGCCACACCTG
CCACTCTCGCTGATCCTCTCTGTGGTGACCTCT GCCCTCGTGGCCGCCC
TGGTCCTGGCTTTCTCCGGCATCATGATTGTGTACCGCCGGAAGCACCAG
GAGCTGCAAG CCATGCAGATGGAGCTGCAGAGCCCTGAGTACAAGCTGA
GCAAGCTCCGCACCTCGACCATCATGACCGACTACAA CCCCAACTACTG
CTTTGCTGGCAAGACCTCCTCCATCAGTGACCTGAAGGAGGTGCCGCGGA
AAAACATCACCCTC ATTCGGGGTCTGGGCCATGGCGCCTTTGGGGAGGT
GTATGAAGGCCAGGTGTCCGGAATGCCCAACGACCCAAGCC CCCTGCAA
GTGGCTGTGAAGACGCTGCCTGAAGTGTGCTCTGAACAGGACGAACTGGA
TTTCCTCATGGAAGCCCT GATCATCAGCAAATTCAACCACCAGAACATT
GTTCGCTGCATTGGGGTGAGCCTGCAATCCCTGCCCCGGTTCATC CTGC
TGGAGCTCATGGCGGGGGGAGACCTCAAGTCCTTCCTCCGAGAGACCCGC
CCTCGCCCGAGCCAGCCCTCCT CCCTGGCCATGCTGGACCTTCTGCACG
TGGCTCGGGACATTGCCTGTGGCTGTCAGTATTTGGAGGAAAACCACTT
CATCCACCGAGACATTGCTGCCAGAAACTGCCTCTTGACCTGTCCAGGCC
CTGGAAGAGTGGCCAAGATTGGAGAC TTCGGGATGGCCCGAGACATCTA
CAGGGCGAGCTACTATAGAAAGGGAGGCTGTGCCATGCTGCCAGTTAAGT
GGA TGCCCCCAGAGGCCTTCATGGAAGGAATATTCACTTCTAAAACAGA
CACATGGTCCTTTGGAGTGCTGCTATGGGA AATCTTTTCTCTTGGATAT
ATGCCATACCCCAGCAAAAGCAACCAGGAAGTTCTGGAGTTTGTCACCAG
TGGAGGC CGGATGGACCCACCCAAGAACTGCCCTGGGCCTGTATACCGG
ATAATGACTCAGTGCTGGCAACATCAGCCTGAAG ACAGGCCCAACTTTG
CCATCATTTTGGAGAGGATTGAATACTGCACCCAGGACCCGGATGTAATC
AACACCGCTTT GCCGATAGAATATGGTCCACTTGTGGAAGAGGAAGAGA
AAGTGCCTGTGAGGCCCAAGGACCCTGAGGGGGTTCCT CCTCTCCTGGT
CTCTCAACAGGCAAAACGGGAGGAGGAGCGCAGCCCAGCTGCCCCACCAC
CTCTGCCTACCACCT CCTCTGGCAAGGCTGCAAAGAAACCCACAGCTGC
AGAGATCTCTGTTCGAGTCCCTAGAGGGCCGGCCGTGGAAGG GGGACAC
GTGAATATGGCATTCTCTCAGTCCAACCCTCCTTCGGAGTTGCACAAGGT
CCACGGATCCAGAAACAAG CCCACCAGCTTGTGGAACCCAACGTACGGC
TCCTGGTTTACAGAGAAACCCACCAAAAAGAATAATCCTATAGCAA AGA
AGGAGCCACACGACAGGGGTAACCTGGGGCTGGAGGGAAGCTGTACTGTC
CCACCTAACGTTGCAACTGGGAG ACTTCCGGGGGCCTCACTGCTCCTAG
AGCCCTCTTCGCTGACTGCCAATATGAAGGAGGTACCTCTGTTCAGGCTA
CGTCACTTCCCTTGTGGGAATGTCAATTACGGCTACCAGCAACAGGGCT
TGCCCTTAGAAGCCGCTACTGCCCCTG GAGCTGGTCATTACGAGGATAC
CATTCTGAAAAGCAAGAATAGCATGAACCAGCCTGGGCCCTGAGCTCGGT
CGCA CACTCACTTCTCTTCCTTGGGATCCCTAAGACCGTGGAGGAGAGA
GAGGCAATGGCTCCTTCACAAACCAGAGACC AAATGTCACGTTTTGTTT
TGTGCCAACCTATTTTGAAGTACCACCAAAAAAGCTGTATTTTGAAAATG
CTTTAGAA AGGTTTTGAGCATGGGTTCATCCTATTCTTTCGAAAGAAGA
AAATATCATAAAAATGAGTGATAAATACAAGGCCC AGATGTGGTTGCAT
AAGGTTTTTATGCATGTTTGTTGTATACTTCCTTATGCTTCTTTCAAATT
GTGTGTGCTCTG CTTCAATGTAGTCAGAATTAGCTGCTTCTATGTTTCA
TAGTTGGGGTCATAGATGTTTCCTTGCCTTGTTGATGTG GACATGAGCC
ATTTGAGGGGAGAGGGAACGGAAATAAAGGAGTTATTTGTAATGACTAAA
A

SEQ ID NO:2 >Reverse Complement of SEQ ID NO:1

TTTTAGTCATTACAAATAACTCCTTTATTTCCGTTCCCTCTCCCCTCAAA
TGGCTCATGTCCACATCAACAAGGCAAGGAAACATCTATGACCCCAACT
ATGAAACATAGAAGCAGCTAATTCTGA CTACATTGAAGCAGAGCACACA
CAATTTGAAAGAAGCATAAGGAAGTATACAACAAACATGCATAAAAACCT
TATG CAACCACATCTGGGCCTTGTATTTATCACTCATTTTTATGATATT
TTCTTCTTTCGAAAGAATAGGATGAACCCAT GCTCAAAACCTTTCTAAA
GCATTTTCAAAATACAGCTTTTTTGGTGGTACTTCAAAATAGGTTGGCAC
AAAACAAA ACGTGACATTTGGTCTCTGGTTTGTGAAGGAGCCATTGCCT
CTCTCTCCTCCACGGTCTTAGGGATCCCAAGGAAG AGAAGTGAGTGTGC
GACCGAGCTCAGGGCCCAGGCTGGTTCATGCTATTCTTGCTTTTCAGAAT
GGTATCCTCGTA ATGACCAGCTCCAGGGGCAGTAGCGGCTTCTAAGGGC
AAGCCCTGTTGCTGGTAGCCGTAATTGACATTCCCACAA GGGAAGTGAC
GTAGCCTGAACAGAGGTACCTCCTTCATATTGGCAGTCAGCGAAGAGGGC
TCTAGGAGCAGTGAGG CCCCCGGAAGTCTCCCAGTTGCAACGTTAGGTG
GGACAGTACAGCTTCCCTCCAGCCCCAGGTTACCCCTGTCGTG TGGCTC
CTTCTTTGCTATAGGATTATTCTTTTTGGTGGGTTTCTCTGTAAACCAGG
AGCCGTACGTTGGGTTCCAC AAGCTGGTGGGCTTGTTTCTGGATCCGTG
GACCTTGTGCAACTCCGAAGGAGGGTTGGACTGAGAGAATGCCATAT TC
ACGTGTCCCCCTTCCACGGCCGGCCCTCTAGGGACTCGAACAGAGATCTC
TGCAGCTGTGGGTTTCTTTGCAGC CTTGCCAGAGGAGGTGGTAGGCAGA
GGTGGTGGGGCAGCTGGGCTGCGCTCCTCCTCCCGTTTTGCCTGTTGAGA
G ACCAGGAGAGGAGGAACCCCCTCAGGGTCCTTGGGCCTCACAGGCACT
TTCTCTTCCTCTTCCACAAGTGGACCAT ATTCTATCGGCAAAGCGGTGT
TGATTACATCCGGGTCCTGGGTGCAGTATTCAATCCTCTCCAAAATGATG
GCAAA GTTGGGCCTGTCTTCAGGCTGATGTTGCCAGCACTGAGTCATTA
TCCGGTATACAGGCCCAGGGCAGTTCTTGGGT GGGTCCATCCGGCCTCC
ACTGGTGACAAACTCCAGAACTTCCTGGTTGCTTTTGCTGGGGTATGGCA
TATATCCAA GAGAAAAGATTTCCCATAGCAGCACTCCAAAGGACCATGT
GTCTGTTTTAGAAGTGAATATTCCTTCCATGAAGGC CTCTGGGGGCATC
CACTTAACTGGCAGCATGGCACAGCCTCCCTTTCTATAGTAGCTCGCCCT
GTAGATGTCTCGG GCCATCCCGAAGTCTCCAATCTTGGCCACTCTTCCA
GGGCCTGGACAGGTCAAGAGGCAGTTTCTGGCAGCAATGT CTCGGTGGA
TGAAGTGGTTTTCCTCCAAATACTGACAGCCACAGGCAATGTCCCGAGCC
ACGTGCAGAAGGTCCAG CATGGCCAGGGAGGAGGGCTGGCTCGGGCGAG
GGCGGGTCTCTCGGAGGAAGGACTTGAGGTCTCCCCCCGCCATG AGCTC
CAGCAGGATGAACCGGGGCAGGGATTGCAGGCTCACCCCAATGCAGCGAA
CAATGTTCTGGTGGTTGAATT TGCTGATGATCAGGGCTTCCATGAGGAA
ATCCAGTTCGTCCTGTTCAGAGCACACTTCAGGCAGCGTCTTCACAGC C
ACTTGCAGGGGGCTTGGGTCGTTGGGCATTCCGGACACCTGGCCTTCATA
CACCTCCCCAAAGGCGCCATGGCCC AGACCCCGAATGAGGGTGATGTTT
TTCCGCGGCACCTCCTTCAGGTCACTGATGGAGGAGGTCTTGCCAGCAAA
GC AGTAGTTGGGGTTGTAGTCGGTCATGATGGTCGAGGTGCGGAGCTTG
CTCAGCTTGTACTCAGGGCTCTGCAGCTC CATCTGCATGGCTTGCAGCT
CCTGGTGCTTCCGGCGGTACACAATCATGATGCCGGAGAAAGCCAGGACC
AGGGCG GCCACGAGGGCAGAGGTCACCACAGAGAGGATCAGCGAGAGTG
GCAGGTGTGGCTCCGGGGTGGGTGACACAATGC AGGAGACGCCATCCTC
AGCCAGCACCGTCCCGTGGTCACAGAAGCAGATGACCTTGTGGCTTTCAG
GGTCCATGTG ACATTCGTCTACCTCACAGTGACTGCAGTTTAGATAATG
CTTAATATTCACTTCCCCGTGGCCTTCCATCACTTTT AAAGCTGGGGTG
TACAGGATGCCCAGTGGACTGATGAAGGAAACCCCATCTTCCCCATCCAT
TTCGGGGTCATTGT TTGAGGCTGCATTGCCGCCTATATATCCTCCGCCT
CCTCCACCTGAGGAGCACCCCCCTCCACCCCCTCCGAAACC CCCTCTTG
TCTCCCACCCCCACTTCTTCATGGCCTGGGGGCAGGAATGTCCTCCGGTG
GCACCCTCCTGCAAAGAT TTTCCGGCCCAGAGCAAGGAAGTGTTATCAT
TCCAGCCACCTCCACCACCTGCGGCTCCGGAATTGCCGTTTAGCC CTAG
AACCGAGGAGTTATTCTCCAGTCTCTCTGGGTGGAACGTGTCTGTCTTGG
CCCCGTAGGCCCTGCCACCACC TCCGGCTGCAATGATCAGGGGCACCGG
CACTCCATCCTTCATCTTAAATACGTAGGTGGCTCCACCCCCTCCTCCT
CCGCCTCCTGCCCACTCATGCACGCTTCTGTTCACACGGATTTCTTCTTC
TATCACATTGTTCTCTCCAATGCAGA CTTTCTGGATTAACTGGTTTGTA
CTGGGGCAGGCGTCCTCTCCCTGCTGCCCAACCAGGATGTACAGCATGTC
ATC CTTCTCCAGGTTGAAGATGCCCAGCACAGACACGCCGTGGGACCGC
ATCATGGTGTTCTTCCCGCCTTTCCCGCCA GCAGCTCCGTAGCCCGAGA
TGCTGTAGGTGTCGGTGGCTGGCACCTTCCAGATCTGGATGCCTTTCAGG
GGGCCCT CGCTCCCCACCTCCACGCTCAGGTTGGAGTTCTGGTAGGCGT
TGTTGCACTGTGCCTGGGTGGGGCCATGGGGCCC GCTGGCCCCACATGT
GGTGAACAGCCAATGAACTGTAGGGTCAAAGATGGGGGTCTGTCTTGGTG
AATTTTCCCCG GGTTTCAGCTCCTTGTTTGGGTTTCTCTCAAACAGGTT
TCTTGATTTGGGTGCTGTATTCTGCAGGATCTTGTCCT CTCCGCTAATG
GTGAGGTAGCAGTCCAGGCTGATGGAGATATTGTCAAAAGCCACGATGGC
TCTGGATCCTTGTCC CCACCATGCGACCATCTGCAGCCAGAACCTGTCA
GACACATCGAGGAGAGGCAACACCATCCACTGCCACAGGCTC AAGCCTT
CATAGGCGGCGACATGCCAGACCATCCTGCCTTGCTCCTTCCCGGTTTTG
TTCTCCACTAGCACCAAGG ACACGTTTCCCCTCAAGACTCCACGAATGA
GCCAGGACATTCGGAGCTCACATGGAGAGCTCTTGATCGGTGCAGG AAA
CGTAGCACTGGTCACTGTAGCACTTTCAGAAGCGGGGACATCAGTGGTAC
TGAGCAATAGAGCATGGTCTTGG TGGTCCTGGAACCGGGCATCCTTTAG
GGTCCTGACCTGCCATTGAGGAGTGTGGGGTGACAGTGTGCCTTGGGTCC
AGCCACAGAAGCCATCTTCAAAGTTGCAGTAAAAACCCACAGGCAGTTT
CCGGCACATCTGGCTCTCATCTTCTCC CTGGGCACAGTCCTGGTGGAAG
TCACAGGCCTGCCCAAGCTGGAGGACTGTCCCATTCCAACAAGTGAAGGA
GCTC TGCAGGGCCATCTTGGAGCCTGGGGATGTTCCTTCACTGCAGTTC
TTCAGGGCAAAGAAGTCCACTGCAGACAAGC TGCGGTTTCCACTGGAGA
TGTATTCCAGGGCCACTCGAAATGGGTTGTCTGGACGCCCGATTCTTCCC
TGGAGCAC TGTCCAACCATGCTTCCCTGGAGTGGGCATCAGGAGGATCT
CTCTTGCAGCCTCGTTGTGGGGCAGCAGCTGGGCA ATGTACCTTCCAGA
GGGCTGCAGGTGCCTGTGCACCGAGACGGCCAGTGTGCAGTGCTCACTGC
TGCTCCTCATCC ACGGACTCAGGATGGTGTGCTTGGAGTCAGCTGAGGT
GTTGAGAAGGAGAAAGGAGCCTCTGGGCATCTCCTTAGA ACGCTCTGCC
CCAGGCCCATCCAGCAAGTCCATCTGGGAGGCCTCCTCGGAGGGGATGCG
GCGCCAGGACCAGCTC TGGTTCCTGAGGTCATGCAGTGGAGGGGAATAC
TCCAGCTCACAGGGGAAGTCAAAGCTGCACTCCAGACCATATC GGCTGC
GATGAGACAGGAAAGGGAAGGAGTCTTTCATTATCCAGGTGAGATTCCAT
GTAAAATAATCAGGAGAAGG AGAAGGCATGTTTGTTGGTGATTCCAAGG
AGCTATGACCAGTCCCGAAGATCTGGAAGAGAAGGCGGGGCTGGGAG GC
GCGAATTGCCGCGGACAGCCTTCCCTCTCTGCCCACTTCCGACGCCTTCT
TCTCGGGCATCAGGCGGATCCTCA GTCGCCCTTCGCCTTGGCGAATCCA
CCAACTGAACAGCTCGCTGAGATTGAACTGGAGCAGCCCCACAGCCGCCT
C CCCGGGGGGCCCGACGCAACCCTCCAAGATCGCCTCCTCGCCCAGCTC
CAGCACCAACTGCTTGGCACGCCGGAGC TTGCGCACGGAGCCGCCCTTC
AGCACCCTGGACAGCGTCCGGGCCTCTGCCGGGGCTGGTGAACCGGCGGT
CCAGG AGACCCCCGGCGCCGGCCCCAGCAACCTGAGCAGCGGGGCGCAG
TCCAGAGCTAGCGAGCCGCGGGCCTCGGGCCT GCCAGCCTTCAGCTCCG
AGGAGGATGGTGGCAGCAGTAGGTCCCGGGCGTAGACACGGAAGAGCGAG
GGCACCACG AAGTCAACTGCCAGACTCTTCCTCTGCAGGCGCGAGTAGC
TGAGTGGCTCCCGGGGCTGCAGCGGCGGCCCCGCAG CTGGGGAGCCCGC
GCGCTGGCCGGTCCCCATCCCGGAGCCCACAGCTGCCGTGGAAAGCAGCA
GCGGCAGGAGCCA CAGGAGCCCGATGGCTCCCATCCCGCCGGAGGAGGC
CGTTTACACTGCTCTCCGGGCCCAGCCTCACCCTTCGCTC TCCCCGAGA
TGGGAAGAGGCTCTGAACAGTCCTTGGTACCCAGCGGCTCCTTCCACCTG
ATCTCCAGAGGACTGTG CGTGCGCGCAAGTCTCTTGCTTTCCCCCAACT
GCACGGAGGCGAGCAGGAGTCTAAATGAAACAGACCTGGAAGCT CAGGG
GCGAGTCCAGAGACACTCAAGCACACTGGGCTCACTGGCTGGGACCTTGA
GCCTCCCGCTCTCCGCGCCGA GTGCCGCGCCCCCGTCTGTAGCTCGCTG
CGCTCGGTACAGAGGAACTACTATGGTTGAAGGGAGGTGGCAGTTGGG T
ACCGTCCTCTCCTGCCCCCCGCAGTCGGAGCTGGGGTCTGTCCCCTCTCG
GGGCAGCCTCCAATCTCTGCAACTT TTAAGGCTGAGAACGGCGGCTCCC
AGCTGCTGCACGCTGTCCTGGCCGCCTTTTGCGTTCCTTTTGGCTCCTCC
AA GCTCTTCTGCCCGGTCTGGGCGGGAACCGAGGGCGGAGGCTGCCGTC
TTGCGCACCCTCAAGCTATCTCTCCGCTG CGGGAAGGCTTCGGACTGTC
TGCCTGCTGAACTTCTGGGCGTGAATCCCAGCCCCCGCGCTGCGCAAGTT
TGCAGC GTCCTTGCTCTCACCGGCGCCTCGGCTCCTCAGAGTTCGCAGG
CACTGGAGCGGCCCCGGCGGCAGCAGCTGAGGG CGTTCAGGGCAGGGGC
GCCCGAAATCTTGCCGCCCCCTCCTCACCCATCATCAGCGCCCGCGGCTT
TGGGTGGCCG ACCAGAGGGCGGCCGGAAAGCACCTCGGTGCCCCGCGAC
CCTCCGAACAGAGGCGGCGGGAGGTACCAGCTGCTAC CACCGCTGCCGC
CCCCAGAGCCGCTGGATCGCATCTGCCTGGGCGCCCGCCACTTGCAGCT

SEQ ID NO:3 >XM_005576218.2 PREDICTED: Macaca fascicularis anaplastic lymphoma receptor tyrosine kinase (ALK), mRNA

AGCTGCAAGTGGCGGGCGCCCAGGCAGATGCGATCCAGCAGCTCTGGGGG
CGGCAGCGGCGGTAGCAGCTGGTACCTCCCGCCGCCTCTGTTCGGAGGG
TCGCGGGGCACCGATGTGCTTTCCGGC CGCCCTCTGGCCGGCCACCCAA
AGCCGCGGGCGCTGATAATGGGTGAGGAGGGGTCGGCAAGATTTCGGGAG
CCCC TGCCCTGAACGCCCTCAGCTGCTGCCGCCTGCGAACTCTGAGGAA
CCGAGCGAGGCGCGGGCGACAGCAAGGACGC TGCAAACTTGCGCAGCGC
GGGGGTTCGGATTCACGCCCTAGAAGTTCAGCGGGCAGAGCAGTCCGAAG
CCTTCCCG CAGCGGAGAGATAGCTTGAGGGTGGGCGAGAAGGCAGCCCC
CGTCGTCGGATCCCACCCAGACCGGGCAGAGGAGC TCGGAGGAGCCAAA
AGGAACGCAGGAGGCGGCCAGGACAGCGTGCAGCAGCTGGGAGCCGCCGT
TCTCAGTCTGGA AAGTTGCAGAGATTGGAGGCTGCCCCGAGAGGGGACA
GACCCCAGCTCCGACCGCGGGGGGCAGGAGAGGACGGTA CCCAACTGCC
ACCTCCCTTCAACCATAGTAGTTCCTCTGTGCGGAGCGCAGCGAGCTACT
GACGGGGGAGCGGCAC TCGGCGTGGCGAGCGGGAGACTCAAGGTCCCAG
TCAGTGAGCCCAGCGTGCTTGAGGGTCTCTGGACTCGCCCCTG AGATTA
CAGCTGTGTTTCCTTGAGACTCCTGCTCGCCTCCGTGCAGTTGGGGGAAA
GCAAGAGACTTGCGCTCACG CACAGTCCTCTGGAGATCAGGTGGAAGGA
GCCGCTGGGTACCGAGGACTGTTCAGAGCTTCTTCCCATCTAGGGGA GA
GCGAAGGGCGAGGCTGGGCCTGGAGAGCAGTGTAAACGGCCTCCTTCGGC
GGGATGGGAACCATCGGGCTCCTG TGGCTGCTGCCGCTGCTGCTTTCCA
CTGCAGCTTTGGGCTCTGGGACGGGGACCGGCCAGCGCACGGGATCCCCA
G CTGCGGGGCCGCCGCTGCAGCCCCGGGAGCCGCTCAGCTACTCTCGCC
TGCAGAGGAAGAGTCTGGCAGTGGACTT CGTAGTGCCCTCACTCTTCCG
TGTCTACGCCCGGGACCTGCTGCTGCCGCCATCCCCCTCGGAGCTGAAGG
CTGGC AGGCCCGAGGCGCGAGGCTCGCTCGCTCTGGACTGCGCCCCGCT
GCTCAGGTTGCTGGGGCCGGCGCCCGGGGTCT CCTGGACAGCCGGCTCA
CCCACCCCGGCAGAGGCCCGGACGCTGTCCAGGGTGCTGAAGGGCGGCTC
GGTGCGCAA GCTCCGGCGTGCCAAGCAGCTGGTGCTGGAGCTGGGCGAG
GAGGCGATCTTGGAGGGTTGCGTCGGGCCCCCTGGG GAGGCGGCTGTGG
GGCTGCTCCAGTTCAACCTCAGCGAGCTGTTCAGTTGGTGGATTCGCCAG
GGCGAAGGGCGAC TGAGGATCCGCCTGATGCCCGAGAAGAAGGCGTCGG
AAGTGGGCAGAGAGGGAAGGCTGTCCGCGGCAATCCGCGC CTCCCAGCC
CCGCCTTCTCTTCCAGATCTTCGGGACTGGTCATAGCTCCTTGGAGTCAC
CAACAAACATACCTTCT CCTTCTCCTGATTATTTTACATGGAATCTCAC
CTGGATAATGAAAGACTCCTTCCCTTTCCTGTCTCATCGCAGCC GATAT
GGTCTGGAGTGCAGCTTTGACTTCCCCTGTGAGCTGGAATATTCCCCTCC
ACTGCACGACCTCAGGAACCA GAGCTGGTCCTGGCGCCGTGTTCCCTCC
GAGGAGGCCTCCCAGATGGACTTGCTGGATGGGCCTGAGGCAGAGCGT T
CTAAGGAGATGCCCAGAGGGTCCTTTCTCCTCCTCAACACCTCAGCTGAC
TCCAAGCACACCATCCTAAGTCCGT GGATGAGGAGCAGCAGTGAGCACT
GTACGCTGGCCGTCTCAGTGCACAGGCACCTGCAGCCCTCTGGAAGGTAC
AT TGCCCAGCTGCTGCCCCACAACGAGGCTGCAAGAGAGATCCTCCTGA
TGCCCACCCCAGGGAAGCATGGTTGGACA GTGCTCCAGGGAAGAATCGG
GCGCCCAGACAACCCCTTTCGAGTGGCCCTGGAATACATCTCCAGTGGAA
ACCGCA GCTTGTCTGCAGTGGACTTCTTTGCCCTGAAGAACTGCAGTGA
AGGAACATCCCCAGGCTCCAAGATGGCCCTGCA GAGCTCCTTCACTTGT
TGGAATGGGACAGTCCTCCAGCTTGGGCAGGCCTGTGACTTCCACCAGGA
CTGCGCCCAG GGAGAAGACGAGAGCCAGATATGCCGGAAACTCCCTGTG
GGTTTTTACTGCAACTTCGAAGATGGCTTCTGTGGCT GGACCCAAGGCA
CACTCTCACCTCACACTCCTCAGTGGCAGGTCAGGACTCTAAAGGATGCC
CGGTTCCAGAACCA CCAAGACCATGCTCTATTGCTCAGTACCACTGATG
CCCCCACTTCAGAAAGTGCTACAGTGACCAGTGCTACGTTT CCCGCACC
GATCAAGAGCTCTCCATGTGAGCTCCGAATGTCCTGGCTCATTCGTGGAG
TCCTGAGGGGAAACGTGT CCTTGGTGCTAGTGGAGAACAAAACTGGGAA
GGAGCAAGGCAGGATGGTCTGGCATGTCGCCGCCTATGAAGGCTT GAGC
CTGTGGCAGTGGACGGTGTTGCCTCTCCTCGACGTGTCTGACAGGTTCTG
GCTGCAGATGGTTGCATGGTGG GGACAAGGATCCAGAGCCATCGTGGCT
TTTGACAATATCTCCATCAGCCTGGACTGCTACCTCACCATTAGTGGAG
AGGACAAGATCCTACAGAATACAGCACCCAAATCAAGAAACCTGTTTGAG
AGAAACCCAAACAAGGAGCTGAAACC CGGGGAAAATTCACCACGACAGA
CGCCCATCTTTGACCCTACAGTTCATTGGCTGTTCACCACATGTGGGGCC
AGC GGACCCCATGGTCCCACCCAGGCGCAGTGCAACAACGCCTACCAGA
ACTCCAACCTGAGCGTGGAGGTGGGAAGCG AGGGCCCCCTGAAGGGCAT
CCAGATCTGGAAGGTGCCAGCCACCGACACCTACAGCATCTCGGGCTACG
GAGCTGC TGGCGGGAAAGGCGGGAAGAACACCATGATGCGGTCCCACGG
CGTGTCTGTGCTGGGCATCTTCAACCTGGAGAAA GACGACACGCTGTAC
ATCCTGGTTGGGCAGCAGGGAGAGGACGCCTGCCCCAGTACAAACCAGTT
AATCCAGAAAG TCTGCATTGGAGAGAACAATGTGATAGAAGAAGAAATC
CGTGTGAACAGAAGCGTGCATGAGTGGGCAGGAGGAGG AGGAGGAGGGG
GTGGAGCCACCTACGTATTTAAGATGAAGGATGGAGTGCCGGTGCCTCTG
ATCATTGCAGCCGGA GGTGGCGGCAGGGCCTATGGGGCCAAGACAGACG
CGTTCCACCCAGAGAGACTGGAGAATAACTCCTCGGTTCTAG GGCTGAA
CGGCAATTCCGGAGCCGCAGGTGGTGGAGGTGGCTGGAATGATAACACTT
CCTTGCTCTGGGCCGGAAA ATCTTTGCTGGAGGGTGCCACCGGAGGACA
TTCCTGCCCCCAGGCCATGAAGAAGTGGGGGTGGGAGACAAGAGGG GGT
TTCGGAGGGGGTGGAGGGGGGTGCTCCTCAGGTGGAGGAGGCGGAGGATA
TATAGGCGGCAATGCAGCCTCAA ACAATGACCCCGAAATGGATGGGGAA
GATGGGGTTTCCTTCATCAGTCCACTGGGCATCCTGTACACCCCAGCTTT
AAAAGTGATGGAAGGCCATGGGGAAGTGAATATTAAGCATTATCTAAAC
TGCAGTCACTGTGAGGTAGACGAATGT CACATGGACACTGAAAGCCACA
AGGTCATCTGCTTCTGTGACCACGGGACAGTGCTGGCAGAGGATGGCGTC
TCCT GCATTGTGTCGCCCACCCCGGAGCCGCACCTGCCACTCTCACTGA
TCCTCTCCGTGGTGACCTCTGCCCTCGTGGC TGCCCTGGTCCTGGCTTT
CTCCGGCATCATGATTGTGTACCGCCGGAAGCACCAGGAGCTGCAAGCCA
TGCAGATG GAGCTGCAGAGCCCTGAGTACAAGCTGAGTAAGCTCCGCAC
CTCAACCATCATGACCGACTACAACCCCAACTACT GCTTTGCTGGCAAG
ACCTCCTCCATCAGTGACCTGAAGGAGGTGCCGCGGAAAAACATCACCCT
CATTCGGGGTCT GGGCCATGGCGCCTTTGGGGAGGTGTATGAAGGCCAG
GTGTCCGGAGTGCCCAACGACCCAAGCCCCCTGCAAGTG GCTGTGAAGA
CGCTGCCTGAGGTGTGCTCAGAACAGGACGAACTGGATTTCCTCATGGAA
GCCCTGATCATCAGCA AATTCAACCACCAGAACATCGTTCGCTGCATCG
GGGTGAGCCTGCAGGCCCTGCCCCGGTTCATCCTACTGGAGCT CATGGC
AGGAGGAGACCTCAAGTCCTTCCTCCGAGAGACCCGCCCTCGCCCGAGCC
AGCCCTCCTCCCTGGCCATG CTGGACCTTCTGCATGTGGCTCGGGACAT
TGCCTGTGGCTGTCAGTATTTGGAGGAAAACCACTTCATCCACCGAG AC
ATTGCTGCCAGAAACTGCCTCTTGACCTGTCCAGGCCCTGGAAGAGTGGC
CAAGATTGGAGACTTCGGGATGGC CCGAGACATCTACAGGGCAAGCTAC
TACAGAAAGGGAGGCTGTGCGATGCTGCCAGTTAAGTGGATGCCCCCAGA
G GCCTTCATGGAAGGAATATTCACCTCTAAAACAGACACATGGTCCTTT
GGAGTGCTGCTGTGGGAAATCTTTTCTC TCGGATATATGCCATATCCCA
GCAAAAGCAACCAGGAAGTTCTGGAGTTCGTCACCAGTGGAGGCCGGATG
GACCC ACCCAAGAACTGCCCTGGGCCTGTATACCGGATAATGACTCAGT
GCTGGCAACATCAGCCTGAAGACAGGCCCAAC TTTGCCATCATTTTGGA
GAGGATTGAATACTGCACCCAGGACCCAGATGTAATCAACACTGCTTTGC
CGATAGAAT ACGGTCCACTTGTAGAAGAGGAAGAGAAAGTACCCGTGAG
GCCCAAGGACCCTGAGGGGGTTCCTCCTCTCCTGGT CTCTCAACAGGCG
AAACGGGGGGAGGAGCGCAGCCCAGCTGCCCCACCACCTGTGCCTACCAC
CTCCTCTGGCAAG GCTGCAAAGAAACCCACAGCTGCAGAGGTCTCTGTT
CGAGTCCCTAGAGGGCCGGCCGTGGAAGGGGGACACGTGA ATATGGCAT
TCTCTCAGTCCAACCCCCCTTCGGAGTTGCACAAGGTCCAGGGATCCAGA
AACAAGCCCACCAGCTT GTGGAACCCAACGTACGGCTCCTGGTTTACAG
AGAAACCCACCAAAAAGAATAATCCTCTAGCAAAGAAGGAGCCA CATGA
GAGAGGTAACCTGGGGCTGGAGGGAAGCTGTACTGTCCCACCTAACGTTG
CAACTGGGAGACTTCCGGGGG CCTCACTGCTCCTAGAGCCCTCTTCGCT
GACTGCCAACATGAAGGAGGTACCTCTGTTCAGGCTACGTCACTTCCC T
TGTGGGAATGTCAATTACGGCTACCAGCAACAGGGCTTGCCCTTAGAAGC
CGCTACTGCCCCCGGAGCTGGTCAT TACGAGGACACCATTCTGAAAAGC
AAGAATAGCATGAACCAGCCTGGGCCCTGAGCTCGGTCGCACACTCACTT
CT CTTCCTTAGGATCCCTAAGACCGTGGAGGAGAGAGAGGCAATGGCTC
CTTCACAAACCAGAGACCAAATGTCACTT TTTGTTTTGTGCCAACCTAT
TTTGAAGTGCCACCAAAAAAGCTGTATTTTCAAAACGCTTTAGAAAGGTT
TTGAGC ATGGGTTCATCCTATTCTTTCGAAAGAAGAAAATGTCATTAAA
ATGAGTGATAAATGCAAGGCCCAGATGTGGTTG CATAAGGTTTTTATGC
ATGTTTGTTGTATACTTCCTTATGCTTCTTTTAAATTGTGTGTGCTCTGC
TTCAATGTAG TCAGAATTAGCTGCTTCTATGTTTCATAGTCGGGGTCAT
AGATGTTTCCTTACCTTGTTGATGTGGACATGAGCCA TTTGAGGGGAGA
GGGAACAGAAATAAAGGAGTTATTTGTAATGACTCAGCATGGGGAAAGAC
ATTCTTTACTTGAA AAATCATAGACAACTAAAATGTCACTTTAGGTGAC
GGTTAGATGCTTTTAATTGTGCTGATTCATCACCAATTGTA ACAAATGT
TGTGAGTAGTTCCAGTAGTATAGCAGAAATGTGTATATACTCATCTAAAT
GAAATGCATACATTCCAA GTGCTTTGAGTAGGATAAAGCACCAATACAG
ATCCAGAGATTCAAACATCCTATTAATATACCTCATGCCTTAAGA AAAT
CCTCTTACTAAAAGTGAAAAACTGAGTGTGAGGTTTCCTACTGGATTTAA
AACTCAAGATTTAAACTACTTA TTCAAAAAATTTCCACTAATTAAACTT
TCATCATTTAAAAGCAAAAATTTTAAGCAGCAGTGATACATAGAAGCAT
AGAAACATCCATTTGTGGGCATAAGTGTCAATACATTTTAGGCCAAAACC
TTCTGTTTGTTTCAGTCATGGACTGA CTACCAAACTCAAGTTTATACCT
GTAGAGATGGACAGGGCCCCAGGAGGAACAGGCCGCTGGTGGTCCTAAGG
AGA GGCACTGATCCAGTCTCTTTCCTTCCATGGCTAACCTGCGTTTTCT
CTCCATCATGTGATCCAAGTACACTGGGAA CACAAATGTGCTGAAATAC
ACATAGCATAATTTGTATAGCTGTCAAAGACAAATTAAAACAAAGAATTT
TTTCCCT AGGAGGCAATGAGGAAAGGAATTGTGGAACCCCTGAAGAGTT
GAGGTTGACATTGACATGTTAGAGTGGGAGTTCT GAACCTGGTTTTCAC
AGATGAATTTTACTTCCATGAAAACGCATTGGGAATCTGTTCAATTTTAC
ACACATACTTA TGTGTGTAATGCTTAGAATGCTTTATCAGTAAGTCCTC
ATTTCATCCTCTTTCTGCTTCTCCTCTATCCTCATGCC CGTTTTCTTTC
CTATTGGTAGCTATGCCAAAGCATTCAAGAGGGAAGCATTCTCTTACTAC
TTGCTAGTAACTAAA CATTACCAAACAAAAGGACTGTGTGTGTGTATAT
TATGAAAGGTATATATATACATGCCTGTTTCCGACCCTCTGT TTCACCT
GCATCAAAAGCATCACTTTCAGCGTGGGAAAG

SEQ ID NO:4 >Reverse Complement of SEQ ID NO:3

CTTTCCCACGCTGAAAGTGATGCTTTTGATGCAGGTGAAACAGAGGGTCG
GAAACAGGCATGTATATATATACCTTTCATAATATACACACACACAGTC
CTTTTGTTTGGTAATGTTTAGTTACTA GCAAGTAGTAAGAGAATGCTTC
CCTCTTGAATGCTTTGGCATAGCTACCAATAGGAAAGAAAACGGGCATGA
GGAT AGAGGAGAAGCAGAAAGAGGATGAAATGAGGACTTACTGATAAAG
CATTCTAAGCATTACACACATAAGTATGTGT GTAAAATTGAACAGATTC
CCAATGCGTTTTCATGGAAGTAAAATTCATCTGTGAAAACCAGGTTCAGA
ACTCCCAC TCTAACATGTCAATGTCAACCTCAACTCTTCAGGGGTTCCA
CAATTCCTTTCCTCATTGCCTCCTAGGGAAAAAAT TCTTTGTTTTAATT
TGTCTTTGACAGCTATACAAATTATGCTATGTGTATTTCAGCACATTTGT
GTTCCCAGTGTA CTTGGATCACATGATGGAGAGAAAACGCAGGTTAGCC
ATGGAAGGAAAGAGACTGGATCAGTGCCTCTCCTTAGGA CCACCAGCGG
CCTGTTCCTCCTGGGGCCCTGTCCATCTCTACAGGTATAAACTTGAGTTT
GGTAGTCAGTCCATGA CTGAAACAAACAGAAGGTTTTGGCCTAAAATGT
ATTGACACTTATGCCCACAAATGGATGTTTCTATGCTTCTATG TATCAC
TGCTGCTTAAAATTTTTGCTTTTAAATGATGAAAGTTTAATTAGTGGAAA
TTTTTTGAATAAGTAGTTTA AATCTTGAGTTTTAAATCCAGTAGGAAAC
CTCACACTCAGTTTTTCACTTTTAGTAAGAGGATTTTCTTAAGGCAT GA
GGTATATTAATAGGATGTTTGAATCTCTGGATCTGTATTGGTGCTTTATC
CTACTCAAAGCACTTGGAATGTAT GCATTTCATTTAGATGAGTATATAC
ACATTTCTGCTATACTACTGGAACTACTCACAACATTTGTTACAATTGGT
G ATGAATCAGCACAATTAAAAGCATCTAACCGTCACCTAAAGTGACATT
TTAGTTGTCTATGATTTTTCAAGTAAAG AATGTCTTTCCCCATGCTGAG
TCATTACAAATAACTCCTTTATTTCTGTTCCCTCTCCCCTCAAATGGCTC
ATGTC CACATCAACAAGGTAAGGAAACATCTATGACCCCGACTATGAAA
CATAGAAGCAGCTAATTCTGACTACATTGAAG CAGAGCACACACAATTT
AAAAGAAGCATAAGGAAGTATACAACAAACATGCATAAAAACCTTATGCA
ACCACATCT GGGCCTTGCATTTATCACTCATTTTAATGACATTTTCTTC
TTTCGAAAGAATAGGATGAACCCATGCTCAAAACCT TTCTAAAGCGTTT
TGAAAATACAGCTTTTTTGGTGGCACTTCAAAATAGGTTGGCACAAAACA
AAAAGTGACATTT GGTCTCTGGTTTGTGAAGGAGCCATTGCCTCTCTCT
CCTCCACGGTCTTAGGGATCCTAAGGAAGAGAAGTGAGTG TGCGACCGA
GCTCAGGGCCCAGGCTGGTTCATGCTATTCTTGCTTTTCAGAATGGTGTC
CTCGTAATGACCAGCTC CGGGGGCAGTAGCGGCTTCTAAGGGCAAGCCC
TGTTGCTGGTAGCCGTAATTGACATTCCCACAAGGGAAGTGACG TAGCC
TGAACAGAGGTACCTCCTTCATGTTGGCAGTCAGCGAAGAGGGCTCTAGG
AGCAGTGAGGCCCCCGGAAGT CTCCCAGTTGCAACGTTAGGTGGGACAG
TACAGCTTCCCTCCAGCCCCAGGTTACCTCTCTCATGTGGCTCCTTCT T
TGCTAGAGGATTATTCTTTTTGGTGGGTTTCTCTGTAAACCAGGAGCCGT
ACGTTGGGTTCCACAAGCTGGTGGG CTTGTTTCTGGATCCCTGGACCTT
GTGCAACTCCGAAGGGGGGTTGGACTGAGAGAATGCCATATTCACGTGTC
CC CCTTCCACGGCCGGCCCTCTAGGGACTCGAACAGAGACCTCTGCAGC
TGTGGGTTTCTTTGCAGCCTTGCCAGAGG AGGTGGTAGGCACAGGTGGT
GGGGCAGCTGGGCTGCGCTCCTCCCCCCGTTTCGCCTGTTGAGAGACCAG
GAGAGG AGGAACCCCCTCAGGGTCCTTGGGCCTCACGGGTACTTTCTCT
TCCTCTTCTACAAGTGGACCGTATTCTATCGGC AAAGCAGTGTTGATTA
CATCTGGGTCCTGGGTGCAGTATTCAATCCTCTCCAAAATGATGGCAAAG
TTGGGCCTGT CTTCAGGCTGATGTTGCCAGCACTGAGTCATTATCCGGT
ATACAGGCCCAGGGCAGTTCTTGGGTGGGTCCATCCG GCCTCCACTGGT
GACGAACTCCAGAACTTCCTGGTTGCTTTTGCTGGGATATGGCATATATC
CGAGAGAAAAGATT TCCCACAGCAGCACTCCAAAGGACCATGTGTCTGT
TTTAGAGGTGAATATTCCTTCCATGAAGGCCTCTGGGGGCA TCCACTTA
ACTGGCAGCATCGCACAGCCTCCCTTTCTGTAGTAGCTTGCCCTGTAGAT
GTCTCGGGCCATCCCGAA GTCTCCAATCTTGGCCACTCTTCCAGGGCCT
GGACAGGTCAAGAGGCAGTTTCTGGCAGCAATGTCTCGGTGGATG AAGT
GGTTTTCCTCCAAATACTGACAGCCACAGGCAATGTCCCGAGCCACATGC
AGAAGGTCCAGCATGGCCAGGG AGGAGGGCTGGCTCGGGCGAGGGCGGG
TCTCTCGGAGGAAGGACTTGAGGTCTCCTCCTGCCATGAGCTCCAGTAG
GATGAACCGGGGCAGGGCCTGCAGGCTCACCCCGATGCAGCGAACGATGT
TCTGGTGGTTGAATTTGCTGATGATC AGGGCTTCCATGAGGAAATCCAG
TTCGTCCTGTTCTGAGCACACCTCAGGCAGCGTCTTCACAGCCACTTGCA
GGG GGCTTGGGTCGTTGGGCACTCCGGACACCTGGCCTTCATACACCTC
CCCAAAGGCGCCATGGCCCAGACCCCGAAT GAGGGTGATGTTTTTCCGC
GGCACCTCCTTCAGGTCACTGATGGAGGAGGTCTTGCCAGCAAAGCAGTA
GTTGGGG TTGTAGTCGGTCATGATGGTTGAGGTGCGGAGCTTACTCAGC
TTGTACTCAGGGCTCTGCAGCTCCATCTGCATGG CTTGCAGCTCCTGGT
GCTTCCGGCGGTACACAATCATGATGCCGGAGAAAGCCAGGACCAGGGCA
GCCACGAGGGC AGAGGTCACCACGGAGAGGATCAGTGAGAGTGGCAGGT
GCGGCTCCGGGGTGGGCGACACAATGCAGGAGACGCCA TCCTCTGCCAG
CACTGTCCCGTGGTCACAGAAGCAGATGACCTTGTGGCTTTCAGTGTCCA
TGTGACATTCGTCTA CCTCACAGTGACTGCAGTTTAGATAATGCTTAAT
ATTCACTTCCCCATGGCCTTCCATCACTTTTAAAGCTGGGGT GTACAGG
ATGCCCAGTGGACTGATGAAGGAAACCCCATCTTCCCCATCCATTTCGGG
GTCATTGTTTGAGGCTGCA TTGCCGCCTATATATCCTCCGCCTCCTCCA
CCTGAGGAGCACCCCCCTCCACCCCCTCCGAAACCCCCTCTTGTCT CCC
ACCCCCACTTCTTCATGGCCTGGGGGCAGGAATGTCCTCCGGTGGCACCC
TCCAGCAAAGATTTTCCGGCCCA GAGCAAGGAAGTGTTATCATTCCAGC
CACCTCCACCACCTGCGGCTCCGGAATTGCCGTTCAGCCCTAGAACCGAG
GAGTTATTCTCCAGTCTCTCTGGGTGGAACGCGTCTGTCTTGGCCCCAT
AGGCCCTGCCGCCACCTCCGGCTGCAA TGATCAGAGGCACCGGCACTCC
ATCCTTCATCTTAAATACGTAGGTGGCTCCACCCCCTCCTCCTCCTCCTC
CTGC CCACTCATGCACGCTTCTGTTCACACGGATTTCTTCTTCTATCAC
ATTGTTCTCTCCAATGCAGACTTTCTGGATT AACTGGTTTGTACTGGGG
CAGGCGTCCTCTCCCTGCTGCCCAACCAGGATGTACAGCGTGTCGTCTTT
CTCCAGGT TGAAGATGCCCAGCACAGACACGCCGTGGGACCGCATCATG
GTGTTCTTCCCGCCTTTCCCGCCAGCAGCTCCGTA GCCCGAGATGCTGT
AGGTGTCGGTGGCTGGCACCTTCCAGATCTGGATGCCCTTCAGGGGGCCC
TCGCTTCCCACC TCCACGCTCAGGTTGGAGTTCTGGTAGGCGTTGTTGC
ACTGCGCCTGGGTGGGACCATGGGGTCCGCTGGCCCCAC ATGTGGTGAA
CAGCCAATGAACTGTAGGGTCAAAGATGGGCGTCTGTCGTGGTGAATTTT
CCCCGGGTTTCAGCTC CTTGTTTGGGTTTCTCTCAAACAGGTTTCTTGA
TTTGGGTGCTGTATTCTGTAGGATCTTGTCCTCTCCACTAATG GTGAGG
TAGCAGTCCAGGCTGATGGAGATATTGTCAAAAGCCACGATGGCTCTGGA
TCCTTGTCCCCACCATGCAA CCATCTGCAGCCAGAACCTGTCAGACACG
TCGAGGAGAGGCAACACCGTCCACTGCCACAGGCTCAAGCCTTCATA GG
CGGCGACATGCCAGACCATCCTGCCTTGCTCCTTCCCAGTTTTGTTCTCC
ACTAGCACCAAGGACACGTTTCCC CTCAGGACTCCACGAATGAGCCAGG
ACATTCGGAGCTCACATGGAGAGCTCTTGATCGGTGCGGGAAACGTAGCA
C TGGTCACTGTAGCACTTTCTGAAGTGGGGGCATCAGTGGTACTGAGCA
ATAGAGCATGGTCTTGGTGGTTCTGGAA CCGGGCATCCTTTAGAGTCCT
GACCTGCCACTGAGGAGTGTGAGGTGAGAGTGTGCCTTGGGTCCAGCCAC
AGAAG CCATCTTCGAAGTTGCAGTAAAAACCCACAGGGAGTTTCCGGCA
TATCTGGCTCTCGTCTTCTCCCTGGGCGCAGT CCTGGTGGAAGTCACAG
GCCTGCCCAAGCTGGAGGACTGTCCCATTCCAACAAGTGAAGGAGCTCTG
CAGGGCCAT CTTGGAGCCTGGGGATGTTCCTTCACTGCAGTTCTTCAGG
GCAAAGAAGTCCACTGCAGACAAGCTGCGGTTTCCA CTGGAGATGTATT
CCAGGGCCACTCGAAAGGGGTTGTCTGGGCGCCCGATTCTTCCCTGGAGC
ACTGTCCAACCAT GCTTCCCTGGGGTGGGCATCAGGAGGATCTCTCTTG
CAGCCTCGTTGTGGGGCAGCAGCTGGGCAATGTACCTTCC AGAGGGCTG
CAGGTGCCTGTGCACTGAGACGGCCAGCGTACAGTGCTCACTGCTGCTCC
TCATCCACGGACTTAGG ATGGTGTGCTTGGAGTCAGCTGAGGTGTTGAG
GAGGAGAAAGGACCCTCTGGGCATCTCCTTAGAACGCTCTGCCT CAGGC
CCATCCAGCAAGTCCATCTGGGAGGCCTCCTCGGAGGGAACACGGCGCCA
GGACCAGCTCTGGTTCCTGAG GTCGTGCAGTGGAGGGGAATATTCCAGC
TCACAGGGGAAGTCAAAGCTGCACTCCAGACCATATCGGCTGCGATGA G
ACAGGAAAGGGAAGGAGTCTTTCATTATCCAGGTGAGATTCCATGTAAAA
TAATCAGGAGAAGGAGAAGGTATGT TTGTTGGTGACTCCAAGGAGCTAT
GACCAGTCCCGAAGATCTGGAAGAGAAGGCGGGGCTGGGAGGCGCGGATT
GC CGCGGACAGCCTTCCCTCTCTGCCCACTTCCGACGCCTTCTTCTCGG
GCATCAGGCGGATCCTCAGTCGCCCTTCG CCCTGGCGAATCCACCAACT
GAACAGCTCGCTGAGGTTGAACTGGAGCAGCCCCACAGCCGCCTCCCCAG
GGGGCC CGACGCAACCCTCCAAGATCGCCTCCTCGCCCAGCTCCAGCAC
CAGCTGCTTGGCACGCCGGAGCTTGCGCACCGA GCCGCCCTTCAGCACC
CTGGACAGCGTCCGGGCCTCTGCCGGGGTGGGTGAGCCGGCTGTCCAGGA
GACCCCGGGC GCCGGCCCCAGCAACCTGAGCAGCGGGGCGCAGTCCAGA
GCGAGCGAGCCTCGCGCCTCGGGCCTGCCAGCCTTCA GCTCCGAGGGGG
ATGGCGGCAGCAGCAGGTCCCGGGCGTAGACACGGAAGAGTGAGGGCACT
ACGAAGTCCACTGC CAGACTCTTCCTCTGCAGGCGAGAGTAGCTGAGCG
GCTCCCGGGGCTGCAGCGGCGGCCCCGCAGCTGGGGATCCC GTGCGCTG
GCCGGTCCCCGTCCCAGAGCCCAAAGCTGCAGTGGAAAGCAGCAGCGGCA
GCAGCCACAGGAGCCCGA TGGTTCCCATCCCGCCGAAGGAGGCCGTTTA
CACTGCTCTCCAGGCCCAGCCTCGCCCTTCGCTCTCCCCTAGATG GGAA
GAAGCTCTGAACAGTCCTCGGTACCCAGCGGCTCCTTCCACCTGATCTCC
AGAGGACTGTGCGTGAGCGCAA GTCTCTTGCTTTCCCCCAACTGCACGG
AGGCGAGCAGGAGTCTCAAGGAAACACAGCTGTAATCTCAGGGGCGAGT
CCAGAGACCCTCAAGCACGCTGGGCTCACTGACTGGGACCTTGAGTCTCC
CGCTCGCCACGCCGAGTGCCGCTCCC CCGTCAGTAGCTCGCTGCGCTCC
GCACAGAGGAACTACTATGGTTGAAGGGAGGTGGCAGTTGGGTACCGTCC
TCT CCTGCCCCCCGCGGTCGGAGCTGGGGTCTGTCCCCTCTCGGGGCAG
CCTCCAATCTCTGCAACTTTCCAGACTGAG AACGGCGGCTCCCAGCTGC
TGCACGCTGTCCTGGCCGCCTCCTGCGTTCCTTTTGGCTCCTCCGAGCTC
CTCTGCC CGGTCTGGGTGGGATCCGACGACGGGGGCTGCCTTCTCGCCC
ACCCTCAAGCTATCTCTCCGCTGCGGGAAGGCTT CGGACTGCTCTGCCC
GCTGAACTTCTAGGGCGTGAATCCGAACCCCCGCGCTGCGCAAGTTTGCA
GCGTCCTTGCT GTCGCCCGCGCCTCGCTCGGTTCCTCAGAGTTCGCAGG
CGGCAGCAGCTGAGGGCGTTCAGGGCAGGGGCTCCCGA AATCTTGCCGA
CCCCTCCTCACCCATTATCAGCGCCCGCGGCTTTGGGTGGCCGGCCAGAG
GGCGGCCGGAAAGCA CATCGGTGCCCCGCGACCCTCCGAACAGAGGCGG
CGGGAGGTACCAGCTGCTACCGCCGCTGCCGCCCCCAGAGCT GCTGGAT
CGCATCTGCCTGGGCGCCCGCCACTTGCAGCT

SEQ ID NO: 5 >NM_007439.2 Mus musculus anaplastic lymphoma kinase (Alk), mRNA

GTGTTCACGCCCAGAAGTTCAGCGGGCAGGGTGATCGATCCGAAGACTTC
CTGCAGCGGAGGTCACTTGAGGGGGCGCTAGAAAGCAGCCCCCTCCGGT
GGTCCTTGCCTAGACCTGGGAAGGAGC GCAGAGGAGGTGACAGGAGCGG
AGGACGTGGGCAAGACAGTGACCGACTCGGAGCCACGGTTCACAGCCTGG
AAAG TTGCAGAAGATTGGAAGCTAAGAGGAGAGCTCTGGTCGCCGAGGG
CTCCTTGAACGGTACCTAATTGCCACCTCCC TGGTCCCTGAGCAAAGGC
CTCTACAAATGGGGCGCAGCACGGCGAGAGGCGCAGGATCCAGCTGTTGA
GCCCAGGG TGTCTCACTGTCTCCGAACTACCCCCTGACTTTGTCTTCCG
TTTTGCTGAGAACCCTTCTCGCCTCCTTGTAGCTT GGGAAAAGCAAGGG
CGCTCTATAGTGTACACACAGTCCCTGAGATCTAGTGGAAGGAGCCATTC
AGGACCAAGGAC TATTTGGAGCCCTTTCCTGTTTGGGGGAGAGTGAAGG
GCGAGGCTGGACCAGCAAGGGAAGGGAGACTAGTGTAAA CTCGCCCTCC
AGCGGGATGGGAGCTGCTGGGTTCCTGTGGCTGCTGCCTCCACTGCTTTT
GGCAGCAGCCTCGTAC TCCGGAGCTGCAACCGATCAGCGCGCGGGTTCC
CCAGCCTCAGGGCCTCCTCTGCAGCCCCGGGAGCCGCTCAGTT ATTCGC
GCCTGCAGAGGAAGAGTCTGGCAGTGGACTTCGTTGTACCCTCGCTCTTC
CGCGTCTATGCCCGAGACCT GCTGCTACCGCAGCCACGGTCCCCCTCGG
AGCCCGAGGCTGGCGGGCTGGAGGCGCGGGGATCACTGGCCCTGGAT TG
TGAGCCTCTGCTCAGGCTGCTGGGGCCACTGCCTGGAATCTCCTGGGCAG
ATGGAGCCAGTTCTCCTAGTCCCG AGGCGGGTCCGACGCTGTCCAGGGT
GCTGAAGGGAGGCTCGGTGCGCAAGCTCAGGCGTGCCAAACAGCTGGTGC
T GGAGCTGGGCGAGGAGACGATTCTTGAAGGCTGTATTGGTCCCCCAGA
GGAGGTAGCGGCTGTGGGGATACTCCAG TTCAACCTCAGCGAGCTGTTC
AGCTGGTGGATTCTCCACGGCGAAGGGAGGCTGAGGATCCGCCTGATGCC
TGAGA AGAAGGCATCGGAAGTGGGCAGGGAGGGAAGGCTATCCAGTGCG
ATCCGAGCCTCCCAGCCCCGCCTTCTCTTCCA GATCTTCGGGACGGGAC
ACAGCTCCATGGAGTCACCCTCAGAAACGCCTTCTCCTCCTGGTACCTTC
ATGTGGAAT CTGACCTGGACGATGAAAGACTCCTTCCCTTTCCTTTCCC
ACCGCAGTCGATATGGTCTGGAGTGCAGCTTTGACT TCCCCTGTGAGCT
GGAATATTCTCCTCCCCTGCACAACCACGGGAATCAGAGCTGGTCCTGGC
GCCATGTGCCCTC CGAGGAGGCCTCGAGGATGAACTTGCTGGATGGGCC
AGAGGCAGAGCATTCTCAAGAGATGCCCAGAGGCTCCTTC CTCCTCCTG
AACACCTCTGCAGATTCCAAGCATACCATTCTGAGCCCATGGATGAGGAG
CAGTAGTGATCACTGCA CACTGGCTGTCTCCGTGCACAGACATCTACAG
CCTTCGGGGAGATATGTTGCCCAGCTCCTACCCCACAACGAAGC TGGAA
GAGAGATTCTTTTGGTGCCCACCCCAGGGAAGCATGGCTGGACAGTGCTG
CAGGGGAGAGTCGGGCGCCCA GCAAACCCATTTCGAGTGGCTCTGGAAT
ACATCTCCAGTGGCAACCGGAGCTTGTCGGCGGTGGATTTCTTTGCCC T
GAAGAACTGCAGTGAAGGGACATCCCCAGGCTCCAAGATGGCATTGCAGA
GTTCCTTCACTTGTTGGAATGGGAC CGTCCTCCAGCTCGGGCAAGCCTG
TGATTTCCACCAGGACTGTGCCCAAGGAGAAGATGAGGGCCAGCTGTGCA
GT AAACTTCCTGCTGGATTTTACTGTAACTTTGAAAATGGCTTCTGTGG
CTGGACCCAAAGTCCACTCTCACCCCATA TGCCCCGGTGGCAAGTGAGG
ACCCTAAGAGATGCCCATTCCCAGGGCCACCAAGGCCGTGCCCTGTTGCT
CAGCAC CACTGACATCCTCGCTTCTGAAGGTGCAACAGTGACCAGTGCC
ACCTTCCCTGCACCAATGAAAAATTCTCCTTGT GAGCTCCGCATGTCCT
GGCTCATCCGCGGGGTTTTGAGAGGAAACGTATCTCTGGTGCTGGTGGAG
AACAAAACCG GAAAGGAGCAAAGCCGGACTGTCTGGCATGTCGCCACTG
ACGAAGGCTTAAGCCTGTGGCAGCATACAGTGCTGTC CCTCCTCGATGT
GACTGACAGGTTCTGGCTGCAGATAGTCACATGGTGGGGTCCAGGATCCA
GGGCAACCGTGGGA TTTGACAACATTTCCATCAGCCTCGACTGCTACCT
TACCATCAGTGGAGAGGAGAAAATGTCCCTGAATTCAGTAC CCAAATCT
AGAAATCTGTTTGAGAAAAACCCAAACAAGGAGTCAAAATCCTGGGCAAA
CATATCAGGACCAACTCC CATCTTCGACCCTACAGTTCACTGGCTGTTC
ACCACGTGTGGGGCCAGTGGACCTCATGGCCCCACCCAGGCACAG TGCA
ACAACGCCTACCAGAATTCCAACTTGAGCGTGGTGGTGGGAAGTGAAGGG
CCCTTGAAGGGAGTCCAGATTT GGAAAGTGCCAGCTACTGACACCTACA
GTATCTCGGGCTACGGAGCAGCTGGCGGGAAAGGTGGGAAAAACACCAT
GATGCGGTCCCATGGCGTGTCTGTCCTGGGCATCTTCAATCTGGAGAAAG
GTGACACACTCTACATCCTTGTCGGT CAGCAAGGGGAGGATGCCTGTCC
CAGGGCAAACCAACTAATCCAGAAAGTCTGTGTGGGTGAGAACAATGTCA
TAG AAGAAGAGATCCGAGTGAACAGAAGCGTGCACGAGTGGGCAGGAGG
AGGAGGAGGTGGGGGTGGAGCCACCTACGT GTTTAAGATGAAAGATGGC
GTGCCTGTACCCCTGATCATTGCAGCTGGTGGTGGTGGCAGGGCCTATGG
GGCCAAG ACAGAAACGTTCCACCCAGAGAGACTGGAGAGTAACTCCTCG
GTTCTAGGGCTGAACGGCAATTCCGGAGCCGCAG GTGGTGGAGGCGGCT
GGAATGATAACACTTCCTTGCTCTGGGCCGGAAAGTCTTTGCTGGAGGGT
GCCGCCGGAGG ACATTCCTGCCCCCAGGCCATGAAGAAGTGGGGGTGGG
AGACAAGAGGGGGTTTCGGAGGGGGTGGAGGGGGGTGC TCCTCAGGTGG
AGGAGGCGGAGGATATATAGGTGGCAACGCAGCATCAAACAATGACCCCG
AAATGGATGGGGAAG ATGGGGTTTCCTTCATCAGTCCATTGGGTATCCT
GTACACCCCGGCCTTAAAAGTGATGGAGGGCCACGGGGAAGT GAATATC
AAGCATTATCTAAACTGCAGTCACTGCGAGGTAGACGAATGTCACATGGA
CCCCGAGAGCCACAAAGTC ATCTGCTTCTGTGATCATGGGACCGTGCTG
GCTGATGATGGTGTCTCCTGCATTGTGTCACCCACCCCGGAGCCCC ACC
TGCCGCTCTCATTGATCCTCTCCGTCGTGACCTCTGCCCTGGTGGCTGCC
CTTGTTCTGGCATTCTCCGGCAT CATGATTGTGTACCGTCGGAAGCACC
AGGAGTTGCAGGCTATGCAGATGGAACTGCAGAGCCCCGAGTATAAGCTG
AGCAAGCTACGGACCTCGACCATCATGACCGACTACAACCCCAACTACT
GCTTCGCTGGCAAGACTTCCTCCATCA GTGACCTGAAAGAAGTGCCACG
GAAAAACATCACACTCATCCGGGGCCTAGGCCATGGCGCATTTGGGGAGG
TGTA TGAAGGCCAGGTGTCTGGAATGCCCAATGACCCAAGCCCTCTACA
AGTGGCTGTAAAGACGCTGCCAGAAGTGTGT TCAGAACAAGATGAGCTG
GACTTTCTCATGGAAGCTCTGATCATCAGCAAATTCAACCACCAGAATAT
TGTTCGCT GCATCGGGGTGAGTCTACAAGCCCTGCCCCGCTTCATCCTG
CTGGAACTCATGGCTGGCGGAGACCTCAAGTCCTT CCTCAGGGAGACAC
GCCCTCGCCCGAACCAACCCACCTCCCTGGCCATGCTGGACCTTCTGCAT
GTGGCTCGGGAC ATTGCCTGTGGCTGTCAGTACCTAGAGGAGAATCACT
TTATCCACCGGGATATTGCTGCTAGAAACTGTCTGTTGA CCTGCCCAGG
AGCTGGAAGAATAGCAAAGATTGGAGACTTTGGGATGGCCCGAGATATCT
ACAGGGCCAGCTACTA CCGAAAGGGAGGCTGCGCCATGCTGCCGGTCAA
GTGGATGCCCCCTGAAGCCTTCATGGAAGGGATATTTACCTCT AAAACA
GACACATGGTCTTTTGGAGTGTTGCTATGGGAAATATTTTCTCTTGGATA
TATGCCGTACCCCAGCAAGA GCAACCAGGAAGTTCTGGAGTTTGTCACC
AGCGGAGGACGGATGGACCCGCCTAAGAACTGCCCCGGGCCTGTATA CC
GGATAATGACGCAGTGCTGGCAGCATCAGCCTGAAGACAGACCCAACTTC
GCCATCATTTTGGAGAGGATCGAA TACTGCACCCAGGACCCCGATGTGA
TCAACACAGCTCTGCCCATCGAATACGGTCCAGTAGTAGAAGAGGAGGAG
A AAGTGCCCATGCGCCCCAAAGACCCCGAGGGGATGCCACCTTTGCTGG
TGTCTCCCCAGCCTGCGAAGCACGAGGA GGCGTCCGCAGCTCCCCAGCC
CGCAGCCCTGACGGCACCAGGCCCATCGGTGAAGAAGCCCCCGGGTGCGG
GTGCG GGCGCGGGCGCGGGTGCGGGTGCCGGCCCGGTGCCCCGAGGTGC
GGCCGATCGGGGCCACGTGAACATGGCTTTCT CTCAGCCCAACCCTCCC
CCGGAGCTGCACAAAGGCCCGGGATCCAGAAACAAGCCGACCAGCCTGTG
GAACCCCAC CTACGGCTCGTGGTTCACCGAGAAGCCTGCCAAAAAGACC
CATCCTCCGCCAGGCGCCGAGCCGCAGGCGCGGGCA GGAGCGGCCGAGG
GTGGCTGGACCGGGCCGGGCGCGGGGCCCCGCAGAGCCGAGGCAGCGCTG
CTGCTAGAGCCAT CGGCGCTGAGCGCCACCATGAAGGAGGTGCCGCTGT
TCAGGCTGCGCCACTTCCCCTGCGGCAATGTCAACTATGG TTACCAGCA
ACAGGGTCTCCCCTTGGAAGCCACAGCCGCGCCAGGGGACACCATGCTGA
AAAGCAAGAATAAGGTC ACCCAGCCGGGGCCCTGAGCCCTGTACTCCAC
TAGCTTCTCCTCCTGGCGGAGCCGGAGCCCACCCAGAGGGAGAT GGACA
GGATGGCTCCACCACAAACCCAAGACCAAAACTTTCATTTTTGTGCCAAC
TTGTTTTGAAGTGCCACATTT TAAAAAAAGGAAACTTGTGTTTTTAAGA
TGTGTTAGAAGGTTTTTTGAGCATGGGTTCATCTATCCTCTCAAAAGA A
GAAAATGCCATTCTTTAAAAAAGAAAAAAAAGCAATCAGTGCAAGGCCCA
GATTGGTTGCGCCAAGTTTTCGTGC ATGGTCTGCTGTACAGTCCCCTAA
GGCTTCTTTCCGATTTTTGTGTGCGCTCTGCTTCCGCGTAGTCAGAAATA
GC TGCTTCCATGTCTCATAGGGGGAGTCCTAGGTGTTTCCTTTGCCTTA
TGAATATGAACCACTCGAGGGGCGGGCGA GGGAACAGAAATAAAG

SEQ ID NO:6 >Reverse Complement of SEQ ID NO:5

CTTTATTTCTGTTCCCTCGCCCGCCCCTCGAGTGGTTCATATTCATAAGG
CAAAGGAAACACCTAGGACTCCCCCTATGAGACATGGAAGCAGCTATTT
CTGACTACGCGGAAGCAGAGCGCACAC AAAAATCGGAAAGAAGCCTTAG
GGGACTGTACAGCAGACCATGCACGAAAACTTGGCGCAACCAATCTGGGC
CTTG CACTGATTGCTTTTTTTTCTTTTTTAAAGAATGGCATTTTCTTCT
TTTGAGAGGATAGATGAACCCATGCTCAAAA AACCTTCTAACACATCTT
AAAAACACAAGTTTCCTTTTTTTAAAATGTGGCACTTCAAAACAAGTTGG
CACAAAAA TGAAAGTTTTGGTCTTGGGTTTGTGGTGGAGCCATCCTGTC
CATCTCCCTCTGGGTGGGCTCCGGCTCCGCCAGGA GGAGAAGCTAGTGG
AGTACAGGGCTCAGGGCCCCGGCTGGGTGACCTTATTCTTGCTTTTCAGC
ATGGTGTCCCCT GGCGCGGCTGTGGCTTCCAAGGGGAGACCCTGTTGCT
GGTAACCATAGTTGACATTGCCGCAGGGGAAGTGGCGCA GCCTGAACAG
CGGCACCTCCTTCATGGTGGCGCTCAGCGCCGATGGCTCTAGCAGCAGCG
CTGCCTCGGCTCTGCG GGGCCCCGCGCCCGGCCCGGTCCAGCCACCCTC
GGCCGCTCCTGCCCGCGCCTGCGGCTCGGCGCCTGGCGGAGGA TGGGTC
TTTTTGGCAGGCTTCTCGGTGAACCACGAGCCGTAGGTGGGGTTCCACAG
GCTGGTCGGCTTGTTTCTGG ATCCCGGGCCTTTGTGCAGCTCCGGGGGA
GGGTTGGGCTGAGAGAAAGCCATGTTCACGTGGCCCCGATCGGCCGC AC
CTCGGGGCACCGGGCCGGCACCCGCACCCGCGCCCGCGCCCGCACCCGCA
CCCGGGGGCTTCTTCACCGATGGG CCTGGTGCCGTCAGGGCTGCGGGCT
GGGGAGCTGCGGACGCCTCCTCGTGCTTCGCAGGCTGGGGAGACACCAGC
A AAGGTGGCATCCCCTCGGGGTCTTTGGGGCGCATGGGCACTTTCTCCT
CCTCTTCTACTACTGGACCGTATTCGAT GGGCAGAGCTGTGTTGATCAC
ATCGGGGTCCTGGGTGCAGTATTCGATCCTCTCCAAAATGATGGCGAAGT
TGGGT CTGTCTTCAGGCTGATGCTGCCAGCACTGCGTCATTATCCGGTA
TACAGGCCCGGGGCAGTTCTTAGGCGGGTCCA TCCGTCCTCCGCTGGTG
ACAAACTCCAGAACTTCCTGGTTGCTCTTGCTGGGGTACGGCATATATCC
AAGAGAAAA TATTTCCCATAGCAACACTCCAAAAGACCATGTGTCTGTT
TTAGAGGTAAATATCCCTTCCATGAAGGCTTCAGGG GGCATCCACTTGA
CCGGCAGCATGGCGCAGCCTCCCTTTCGGTAGTAGCTGGCCCTGTAGATA
TCTCGGGCCATCC CAAAGTCTCCAATCTTTGCTATTCTTCCAGCTCCTG
GGCAGGTCAACAGACAGTTTCTAGCAGCAATATCCCGGTG GATAAAGTG
ATTCTCCTCTAGGTACTGACAGCCACAGGCAATGTCCCGAGCCACATGCA
GAAGGTCCAGCATGGCC AGGGAGGTGGGTTGGTTCGGGCGAGGGCGTGT
CTCCCTGAGGAAGGACTTGAGGTCTCCGCCAGCCATGAGTTCCA GCAGG
ATGAAGCGGGGCAGGGCTTGTAGACTCACCCCGATGCAGCGAACAATATT
CTGGTGGTTGAATTTGCTGAT GATCAGAGCTTCCATGAGAAAGTCCAGC
TCATCTTGTTCTGAACACACTTCTGGCAGCGTCTTTACAGCCACTTGT A
GAGGGCTTGGGTCATTGGGCATTCCAGACACCTGGCCTTCATACACCTCC
CCAAATGCGCCATGGCCTAGGCCCC GGATGAGTGTGATGTTTTTCCGTG
GCACTTCTTTCAGGTCACTGATGGAGGAAGTCTTGCCAGCGAAGCAGTAG
TT GGGGTTGTAGTCGGTCATGATGGTCGAGGTCCGTAGCTTGCTCAGCT
TATACTCGGGGCTCTGCAGTTCCATCTGC ATAGCCTGCAACTCCTGGTG
CTTCCGACGGTACACAATCATGATGCCGGAGAATGCCAGAACAAGGGCAG
CCACCA GGGCAGAGGTCACGACGGAGAGGATCAATGAGAGCGGCAGGTG
GGGCTCCGGGGTGGGTGACACAATGCAGGAGAC ACCATCATCAGCCAGC
ACGGTCCCATGATCACAGAAGCAGATGACTTTGTGGCTCTCGGGGTCCAT
GTGACATTCG TCTACCTCGCAGTGACTGCAGTTTAGATAATGCTTGATA
TTCACTTCCCCGTGGCCCTCCATCACTTTTAAGGCCG GGGTGTACAGGA
TACCCAATGGACTGATGAAGGAAACCCCATCTTCCCCATCCATTTCGGGG
TCATTGTTTGATGC TGCGTTGCCACCTATATATCCTCCGCCTCCTCCAC
CTGAGGAGCACCCCCCTCCACCCCCTCCGAAACCCCCTCTT GTCTCCCA
CCCCCACTTCTTCATGGCCTGGGGGCAGGAATGTCCTCCGGCGGCACCCT
CCAGCAAAGACTTTCCGG CCCAGAGCAAGGAAGTGTTATCATTCCAGCC
GCCTCCACCACCTGCGGCTCCGGAATTGCCGTTCAGCCCTAGAAC CGAG
GAGTTACTCTCCAGTCTCTCTGGGTGGAACGTTTCTGTCTTGGCCCCATA
GGCCCTGCCACCACCACCAGCT GCAATGATCAGGGGTACAGGCACGCCA
TCTTTCATCTTAAACACGTAGGTGGCTCCACCCCCACCTCCTCCTCCTC
CTGCCCACTCGTGCACGCTTCTGTTCACTCGGATCTCTTCTTCTATGACA
TTGTTCTCACCCACACAGACTTTCTG GATTAGTTGGTTTGCCCTGGGAC
AGGCATCCTCCCCTTGCTGACCGACAAGGATGTAGAGTGTGTCACCTTTC
TCC AGATTGAAGATGCCCAGGACAGACACGCCATGGGACCGCATCATGG
TGTTTTTCCCACCTTTCCCGCCAGCTGCTC CGTAGCCCGAGATACTGTA
GGTGTCAGTAGCTGGCACTTTCCAAATCTGGACTCCCTTCAAGGGCCCTT
CACTTCC CACCACCACGCTCAAGTTGGAATTCTGGTAGGCGTTGTTGCA
CTGTGCCTGGGTGGGGCCATGAGGTCCACTGGCC CCACACGTGGTGAAC
AGCCAGTGAACTGTAGGGTCGAAGATGGGAGTTGGTCCTGATATGTTTGC
CCAGGATTTTG ACTCCTTGTTTGGGTTTTTCTCAAACAGATTTCTAGAT
TTGGGTACTGAATTCAGGGACATTTTCTCCTCTCCACT GATGGTAAGGT
AGCAGTCGAGGCTGATGGAAATGTTGTCAAATCCCACGGTTGCCCTGGAT
CCTGGACCCCACCAT GTGACTATCTGCAGCCAGAACCTGTCAGTCACAT
CGAGGAGGGACAGCACTGTATGCTGCCACAGGCTTAAGCCTT CGTCAGT
GGCGACATGCCAGACAGTCCGGCTTTGCTCCTTTCCGGTTTTGTTCTCCA
CCAGCACCAGAGATACGTT TCCTCTCAAAACCCCGCGGATGAGCCAGGA
CATGCGGAGCTCACAAGGAGAATTTTTCATTGGTGCAGGGAAGGTG GCA
CTGGTCACTGTTGCACCTTCAGAAGCGAGGATGTCAGTGGTGCTGAGCAA
CAGGGCACGGCCTTGGTGGCCCT GGGAATGGGCATCTCTTAGGGTCCTC
ACTTGCCACCGGGGCATATGGGGTGAGAGTGGACTTTGGGTCCAGCCACA
GAAGCCATTTTCAAAGTTACAGTAAAATCCAGCAGGAAGTTTACTGCAC
AGCTGGCCCTCATCTTCTCCTTGGGCA CAGTCCTGGTGGAAATCACAGG
CTTGCCCGAGCTGGAGGACGGTCCCATTCCAACAAGTGAAGGAACTCTGC
AATG CCATCTTGGAGCCTGGGGATGTCCCTTCACTGCAGTTCTTCAGGG
CAAAGAAATCCACCGCCGACAAGCTCCGGTT GCCACTGGAGATGTATTC
CAGAGCCACTCGAAATGGGTTTGCTGGGCGCCCGACTCTCCCCTGCAGCA
CTGTCCAG CCATGCTTCCCTGGGGTGGGCACCAAAAGAATCTCTCTTCC
AGCTTCGTTGTGGGGTAGGAGCTGGGCAACATATC TCCCCGAAGGCTGT
AGATGTCTGTGCACGGAGACAGCCAGTGTGCAGTGATCACTACTGCTCCT
CATCCATGGGCT CAGAATGGTATGCTTGGAATCTGCAGAGGTGTTCAGG
AGGAGGAAGGAGCCTCTGGGCATCTCTTGAGAATGCTCT GCCTCTGGCC
CATCCAGCAAGTTCATCCTCGAGGCCTCCTCGGAGGGCACATGGCGCCAG
GACCAGCTCTGATTCC CGTGGTTGTGCAGGGGAGGAGAATATTCCAGCT
CACAGGGGAAGTCAAAGCTGCACTCCAGACCATATCGACTGCG GTGGGA
AAGGAAAGGGAAGGAGTCTTTCATCGTCCAGGTCAGATTCCACATGAAGG
TACCAGGAGGAGAAGGCGTT TCTGAGGGTGACTCCATGGAGCTGTGTCC
CGTCCCGAAGATCTGGAAGAGAAGGCGGGGCTGGGAGGCTCGGATCG CA
CTGGATAGCCTTCCCTCCCTGCCCACTTCCGATGCCTTCTTCTCAGGCAT
CAGGCGGATCCTCAGCCTCCCTTC GCCGTGGAGAATCCACCAGCTGAAC
AGCTCGCTGAGGTTGAACTGGAGTATCCCCACAGCCGCTACCTCCTCTGG
G GGACCAATACAGCCTTCAAGAATCGTCTCCTCGCCCAGCTCCAGCACC
AGCTGTTTGGCACGCCTGAGCTTGCGCA CCGAGCCTCCCTTCAGCACCC
TGGACAGCGTCGGACCCGCCTCGGGACTAGGAGAACTGGCTCCATCTGCC
CAGGA GATTCCAGGCAGTGGCCCCAGCAGCCTGAGCAGAGGCTCACAAT
CCAGGGCCAGTGATCCCCGCGCCTCCAGCCCG CCAGCCTCGGGCTCCGA
GGGGGACCGTGGCTGCGGTAGCAGCAGGTCTCGGGCATAGACGCGGAAGA
GCGAGGGTA CAACGAAGTCCACTGCCAGACTCTTCCTCTGCAGGCGCGA
ATAACTGAGCGGCTCCCGGGGCTGCAGAGGAGGCCC TGAGGCTGGGGAA
CCCGCGCGCTGATCGGTTGCAGCTCCGGAGTACGAGGCTGCTGCCAAAAG
CAGTGGAGGCAGC AGCCACAGGAACCCAGCAGCTCCCATCCCGCTGGAG
GGCGAGTTTACACTAGTCTCCCTTCCCTTGCTGGTCCAGC CTCGCCCTT
CACTCTCCCCCAAACAGGAAAGGGCTCCAAATAGTCCTTGGTCCTGAATG
GCTCCTTCCACTAGATC TCAGGGACTGTGTGTACACTATAGAGCGCCCT
TGCTTTTCCCAAGCTACAAGGAGGCGAGAAGGGTTCTCAGCAAA ACGGA
AGACAAAGTCAGGGGGTAGTTCGGAGACAGTGAGACACCCTGGGCTCAAC
AGCTGGATCCTGCGCCTCTCG CCGTGCTGCGCCCCATTTGTAGAGGCCT
TTGCTCAGGGACCAGGGAGGTGGCAATTAGGTACCGTTCAAGGAGCCC T
CGGCGACCAGAGCTCTCCTCTTAGCTTCCAATCTTCTGCAACTTTCCAGG
CTGTGAACCGTGGCTCCGAGTCGGT CACTGTCTTGCCCACGTCCTCCGC
TCCTGTCACCTCCTCTGCGCTCCTTCCCAGGTCTAGGCAAGGACCACCGG
AG GGGGCTGCTTTCTAGCGCCCCCTCAAGTGACCTCCGCTGCAGGAAGT
CTTCGGATCGATCACCCTGCCCGCTGAAC TTCTGGGCGTGAACAC

SEQ ID NO: 7 >NM_001169101.2 Rattus norvegicus ALK receptor tyrosine kinase (Alk), mRNA

GCAGCTCTGGGGGCGGCAGCGGCGGTGGCAACTGGCTCCTCCCGCTTCTT
CTGTCCAGGGGTTCTCGGGGCAGTAAGACGCTTTCCGTTCGCCCCCTGG
CCAGTCAACAAAACCGCGGGCGCTGCT GGTAGGTAAGGAGGGAGCTGGC
TACGTTGCGGGCTCCTCTGCCCCGAATGTTTTCACTCGGCGGCACGGCCG
GGGC GCTCTGGTGCTTGGGAGTGAACTCCGAGGAACGAAGACGTCGGTG
ACAGCAAGGACGCTGCAAACTTGTGCGCGTG GAGGCAGCGGGACCTCCT
CTCCGCAACGCAGAGCTCGCGTTCACGCCCAGAAGTTCAGCGGGCAGGGT
GATCGATC CGAAGACTTCCTGCAGCGGAGGTCACTTGAGGGGGCGCTAG
AAAGCAGCCCCCTCCGGTGGTCCTTGCCTAGACCT GGGAAGGAGCGCAG
AGGAGGTGACAGGAACGGAGGACGTGGGCAAGACAGTGACCGACTCAGAA
CCGCGGCTCGCA GCCTGGAAAGTTGCAGAAGATTGGAGGCGAAGAGGAG
AGCTCTGGTCACCGAGGGCTCCTTGAACGGTACCTAATT GCCACCTCCC
TCAACCATAGTGATTTCTCTGGTCCCTGAGCAAATGGGGCGCAGCATGGC
GAGAGGCGCAGGATCC AGCTGTTGAACCCAAGGTGTCTCACTGTCTCCG
AACTCCCCCCTGACTTTCTCATCCGTTTTGCTGAGAACCCTTT TTGCCT
CCTTGTAGACTGGGGAAAGCAGGGCGCTCTATAGTGTACACAGTCCTTGA
GACCAAGTGGAAGGAGCCGT TCAGGACCAAGGACTATTTGGAGCCCCTT
TCTGTTTAGGGGAGAGTGAAAGGTGAGGCTGGACCAGGCAGGGAAGG GA
GTCTAGTGTAAACTCGCCCTCCGGAGGGATGGGAGCCCTTGGGTTCCTGT
GGCTGCTGCCGCCGCTGCTTTTGA CAGCAGCCTCGTACTCCGGAGCTGC
AACCGATCAGCGCGCGGGTTCTCCAGCCTCAGGGCCGCCTCTGCAGCCCC
G GGAGCCGCTCAGCTATTCGCGCCTGCAGAGGAAGAGTCTGGCGGTGGA
CTTCGTGGTGCCCTCACTCTTCCGCGTC TATGCCCGAGACCTGCTGCTG
CCGCAGCCACCGTCCCCCTCGGAGCCCGGGGCTGGCGGGCTGGAGGCGCG
GGGAT CACTGGCTCTGGACTGCGACCCTCTGCTCAGGTTGCTGGGGCCA
TCGCCTGGAATCTCCTGGGCAGAGGGAGCCAG TTCTCCTAGTCCGGAGG
CGGCTCCGACGCTGTCCAGGGTGCTGAAGGGCGGCTCGGTGCGCAAGCTC
AGGCGTGCC AAACAGCTGGTGCTGGAGCTGGGCGAGGAGACGATCCTTG
AAGGCTGCATTGGTCCCCCAGAGGAGGCGGCGGCTG TGGGGATACTCCA
GTTCAACCTTAGCGAGCTGTTCAGCTGGTGGATTCTACACGGCGAAGGGC
GGCTGAGGATCCG CCTGATGCCTGAGAAGAAGGCATCGGAAGTGGGCAG
GGAGGGAAGGCTATCCACTGCGATCCGAGCCTCCCAGCCC CGCCTTCTC
TTCCAGATCTTCGGGACCGGTCACAGCTCCTTGGAATCACCCTCAGAAAT
GCCTTCTCCTCCTGGTA ACTTTCTATGGAATCTCACCTGGACAATGAAA
GACTCCTTCCCTTTCCTTTCCCACCGCAGTCGATATGGTCTGGA GTGCA
GCTTCGATTTTCCCTGTGAGCTAGAATACTCCCCTCCCCTCCACACCCAT
GGGAACCAGAGCTGGTCCTGG CGCCGTGTGCCCTCTGAGGAGGCCTCGA
GGATGAACTTGTTGGATGGGCCAGAGGCAGAGCATTTGAAAGAGATGC C
CAGAGGCTCCTTCCTCCTCCTGAACACCTCTGCAGATTCCAAGCATACCA
TCCTGAGCCCCTGGATGAGGAGCAG TAGTGAGCACTGCACGCTGGCTGT
CTCAGTGCACAGACATCTACAGCCTTCTGGGAGATATGTTGCCCAGCTCC
TA CCCCACAATGAAGCTGGAAGAGAGATTCTTCTGGTGCCTACCCCGGG
GAAACATGGCTGGACAGTGCTGCATGGGA GAGTCGGGCGCCCAGAGAAC
CCATTCCGAGTGGCCCTGGAATACATCTCGAGTGGAAACCGGAGCTTGTC
TGCGGT GGATTTCTTTGCCCTGAAGAACTGCAGTGAAGGAACATCCCCA
GGCTCCAAGATGGCCTTGCAGAGTTCCTTCACT TGTTGGAACGGGACAG
TCCTCCAGCTCGGGCAAGCCTGTGACTTCCACCAGAACTGTGCCCAAGGA
GAAGATGAGG GTCAGCTATGCAGTAAACTTCCTGCTGGGTTTTACTGTA
ACTTCGAGGATGGCTTCTGTGGCTGGACCCAAAGTCC ACTCTCACCCCG
TGTGCCCCGATGGCAAGTGAAGACCCTGAAAGATACCCATTCCCAGGGCC
ACCAAGGCCATGCC CTGTTGCTCAGCACCACTGACGACCCCACTTCAGA
AAGTGCAACAGTGACCAGTGCTACATTCCCCGCACCAATGA AGAGCTCT
CCTTGTGAGCTCCGGATGTCCTGGCTCATCCGTGGGGTTCTGAAAGGAAA
TGTATCTTTGGTGCTGGT GGAGAACAAAACTGGAAAGGAGCAAAGCCGG
ACCGTCTGGCATGTTGCCACCAATGAAGGCCTAAGCCTGTGGCAG TGGA
CAGTGTTGTCGCTCCTCGATGTGACTGACAGGTTCTGGCTGCAAATAGTC
ACATGGTGGGGCCCAGGATCCA GGGCAACCGTGGCATTTGACAACATTT
CCATCAGCCTCGACTGCTACCTCACCATCAGTGGGGAGGAGAAAATGTC
CCTGAATGCAGTACCCAAATCCAGAAATCTGTTTGAGAAAAACCCAAACA
AGGAGCCAAAACCCTGGGCAAACATA TCAGGACCAACTCCCATCTTCGA
CCCTACAGTTCACTGGCTGTTCACCACATGTGGGGCCAGTGGCCCTCATG
GCC CCACACAGGCACAGTGCAACAACGCCTACCAGAACTCCAACCTGAG
CGTGGTGGTGGGAAGTGAAGGGCCCTTGAG GGGCATCCAGATTTGGAAA
GTGCCAGCTACTGACACCTACAGTATCTCAGGCTATGGAGCAGCTGGCGG
AAAAGGT GGGAAGAACACCATGATGCGGTCCCATGGCGTGTCTGTCCTG
GGCATCTTCAATCTGGAGAAAGATGACACGCTCT ACATCCTTGTCGGTC
AACAAGGGGAAGATGCCTGTCCCAGGGCAAACCAACTAATCCAGAAAGTA
TGTGTCGGCGA GAACAATGTGATAGAAGAAGAGATCCGAGTGAACAGAA
GCGTTCACGAGTGGGCAGGAGGAGGAGGAGGTGGGGGT GGAGCCACCTA
CGTGTTTAAGATGAAAGACGGCGTGCCCGTGCCCCTGATTATTGCGGCCG
GCGGTGGTGGCAGGG CCTATGGGGCCAAGACAGAAACGTTCCACCCAGA
GAGACTGGAGAATAACTCCTCAGTTCTAGGGCTGAACGGCAA TTCCGGA
GCCGCAGGTGGTGGAGGCGGCTGGAATGATAACACTTCCTTGCTCTGGGC
CGGAAAGTCTTTGTTGGAG GGCGCCGCCGGAGGACATTCCTGCCCCCAG
GCCATGAAGAAGTGGGGGTGGGAGACAAGAGGGGGTTTCGGAGGGG GTG
GAGGGGGGTGCTCCTCAGGTGGAGGAGGCGGAGGATATATAGGTGGCAAC
GCAGCATCAAACAATGACCCCGA AATGGATGGGGAAGATGGGGTTTCCT
TCATCAGTCCATTGGGTATCCTGTATACCCCGGCCTTAAAAGTGATGGAG
GGCCATGGGGAAGTGAATATCAAGCACTATCTAAACTGCAGCCACTGTG
AGGTAGACGAATGTCACATGGACCCCG AGAGCCACAAAGTCATCTGCTT
CTGTGACCATGGGACCGTGCTGGCTGACGATGGTGTCTCCTGCATTGTGT
CGCC CACCCCGGAACCCCACCTGCCGCTCTCATTGATCCTCTCCGTCGT
GACCTCTGCCCTGGTGGCCGCTCTCGTTCTG GCATTCTCCGGCATCATG
ATTGTGTACCGCCGGAAGCACCAGGAGTTGCAAGCTATGCAGATGGAGCT
GCAGAGCC CTGAGTATAAGCTGAGCAAGCTACGGACCTCGACCATCATG
ACAGACTACAACCCCAACTACTGCTTTGCTGGCAA GACTTCATCCATCA
GTGACCTGAAGGAGGTGCCTCGGAAAAACATCACCCTCATCCGGGGCCTA
GGCCATGGCGCA TTTGGGGAGGTGTATGAAGGCCAGGTATCTGGAATGC
CCAATGACCCAAGCCCTCTACAAGTGGCTGTAAAGACGC TGCCGGAAGT
GTGTTCAGAGCAAGATGAGCTGGACTTCCTCATGGAAGCTCTGATCATCA
GTAAATTCAACCACCA GAACATTGTCCGCTGCATCGGGGTGAGTCTGCA
AGCCCTGCCCCGCTTCATCCTGCTGGAGCTCATGGCTGGTGGA GACCTC
AAGTCCTTCCTCAGGGAGACACGTCCTCGCCCGAACCAGCCCACCTCCCT
GGCCATGTTGGATCTTCTGC ACGTAGCTCGGGACATTGCCTGTGGCTGT
CAGTACCTGGAGGAAAATCACTTTATCCACCGCGATATTGCTGCTAG AA
ACTGTCTGCTGACCTGTCCGGGAGCTGGGAGGATAGCGAAGATCGGAGAC
TTCGGGATGGCCCGAGATATCTAC AGGGCCAGCTACTACAGAAAGGGAG
GCTGTGCCATGCTGCCAGTGAAGTGGATGCCCCCGGAGGCCTTCATGGAA
G GAATATTTACTTCTAAAACAGATACATGGTCTTTTGGAGTGTTGCTGT
GGGAAATATTTTCTCTTGGATATATGCC ATACCCCAGCAAGAGCAACCA
GGAAGTTCTGGAGTTTGTCACCAGTGGAGGACGGATGGACCCACCTAAGA
ACTGC CCCGGACCTGTATACCGGATAATGACTCAGTGCTGGCAGCATCA
GCCTGAGGACAGACCCAACTTTGCCATCATTC TGGAGAGGATCGAATAC
TGCACCCAGGACCCCGATGTCATCAATACAGCTCTGCCCATCGAATATGG
CCCCGTAGT AGAGGAGGAGGAGAAAGTGCCCATGCGCCCCAAAGACCCT
GAGGGGATGCCTCCTCTGCTGGTGTCTCCCCAGTCT GCGAAGCACGAGG
AGGCGTCCTCAGCCCCCCAACCCTCAGCCCTGGCAGCGCCAGGCCCGCTG
GTGAAGAAGCCCT CGGGTGCAGGCGCGGGAGCGGGTGCCGGTCCGGTGC
CCCGAGGTGCAGCCGATCGGGGCCACGTGAACATGGCGTT CTCTCAGCC
CAACCCTCCCCCAGAGCTGCACAAAGGCCCGGGCTCCAGAAACAAGCCGA
CCAGCCTGTGGAACCCC ACCTACGGCTCGTGGTTCACCGAGAAGCCAGC
CAAAAAGACCCATCCCCCACCGGGCGCGGAGCCGCAGGCCCGGG CAGGA
GCGGCGGAGGGTGGCTGGACCGGGCCGGGTGCGGGGCCCCGCAGAGCCGA
GGCTGCGCTGCTGCTCGAGCC GTCGGCGCTCAGCGCCACCATGAAGGAG
GTGCCCCTGTTCAGGCTGCGCCACTTCCCCTGCGGCAACGTCAACTAT G
GTTACCAGCAACAGGGTCTCCCCTTGGAAGCCACTGCTGCCCCGGGGGAC
ACCGTGCTGAAAAGCAAGACTAAGG TCACCCAGCCAGGGCCCTGAGCCC
GGCGTCCCACTAGCTTTTCTCCCTGGCTGGAGCTGAAGCCCACCGGGAGG
GA GACGGACAGAATGACTCCACCACAATTCCGAGACCAAATGTCACTCA
CTTTCATTTTTGTGCCAACTTGTTTTGAA GTGCCACATTTAAAAGAGAA
AAAGAAGAAAAAAAAAAGAGGAAAACTTGTTTTCAAGATGTGTTAGAAAG
GTTTTT GAGCATGGGTTCATCCATCCTCAAAGAAGAAAATGCCGTTCTT
AAGAAAGCGATCAGTACAAGGCCCAGATTGGTT GCGTAGTTTTGGTGCA
TGATCTGCTGTACAGTCCCCTCAGGCTTCTTTCCGATTTGTGTGTGCTCT
CTGCTTCCGT GTAGTCAGAAATAGCTGCCTCCATGTCTCATAGGGGGAG
TCGTAGGTGTTTCCTTGCCTTGTGGATATGAACCATT CGAGGGGCGAGG
GAACAGAAATAAAGGAATTAATTTCAATGACTCAGCATGAGGAGAGACGT
TATTTACGTGGAAA AGAAATATCATACACTGTCATCGCTGTGGGTGGTG
GTTAGAGGCTTTTAATTGTTCTAACTCATTGCCTATTGTAA AAAAAAAA
AAAAAA

SEQ ID NO:8 >Reverse Complement of SEQ ID NO:7

TTTTTTTTTTTTTTTTACAATAGGCAATGAGTTAGAACAATTAAAAGCCT
CTAACCACCACCCACAGCGATGACAGTGTATGATATTTCTTTTCCACGT
AAATAACGTCTCTCCTCATGCTGAGTC ATTGAAATTAATTCCTTTATTT
CTGTTCCCTCGCCCCTCGAATGGTTCATATCCACAAGGCAAGGAAACACC
TACG ACTCCCCCTATGAGACATGGAGGCAGCTATTTCTGACTACACGGA
AGCAGAGAGCACACACAAATCGGAAAGAAGC CTGAGGGGACTGTACAGC
AGATCATGCACCAAAACTACGCAACCAATCTGGGCCTTGTACTGATCGCT
TTCTTAAG AACGGCATTTTCTTCTTTGAGGATGGATGAACCCATGCTCA
AAAACCTTTCTAACACATCTTGAAAACAAGTTTTC CTCTTTTTTTTTTC
TTCTTTTTCTCTTTTAAATGTGGCACTTCAAAACAAGTTGGCACAAAAAT
GAAAGTGAGTGA CATTTGGTCTCGGAATTGTGGTGGAGTCATTCTGTCC
GTCTCCCTCCCGGTGGGCTTCAGCTCCAGCCAGGGAGAA AAGCTAGTGG
GACGCCGGGCTCAGGGCCCTGGCTGGGTGACCTTAGTCTTGCTTTTCAGC
ACGGTGTCCCCCGGGG CAGCAGTGGCTTCCAAGGGGAGACCCTGTTGCT
GGTAACCATAGTTGACGTTGCCGCAGGGGAAGTGGCGCAGCCT GAACAG
GGGCACCTCCTTCATGGTGGCGCTGAGCGCCGACGGCTCGAGCAGCAGCG
CAGCCTCGGCTCTGCGGGGC CCCGCACCCGGCCCGGTCCAGCCACCCTC
CGCCGCTCCTGCCCGGGCCTGCGGCTCCGCGCCCGGTGGGGGATGGG TC
TTTTTGGCTGGCTTCTCGGTGAACCACGAGCCGTAGGTGGGGTTCCACAG
GCTGGTCGGCTTGTTTCTGGAGCC CGGGCCTTTGTGCAGCTCTGGGGGA
GGGTTGGGCTGAGAGAACGCCATGTTCACGTGGCCCCGATCGGCTGCACC
T CGGGGCACCGGACCGGCACCCGCTCCCGCGCCTGCACCCGAGGGCTTC
TTCACCAGCGGGCCTGGCGCTGCCAGGG CTGAGGGTTGGGGGGCTGAGG
ACGCCTCCTCGTGCTTCGCAGACTGGGGAGACACCAGCAGAGGAGGCATC
CCCTC AGGGTCTTTGGGGCGCATGGGCACTTTCTCCTCCTCCTCTACTA
CGGGGCCATATTCGATGGGCAGAGCTGTATTG ATGACATCGGGGTCCTG
GGTGCAGTATTCGATCCTCTCCAGAATGATGGCAAAGTTGGGTCTGTCCT
CAGGCTGAT GCTGCCAGCACTGAGTCATTATCCGGTATACAGGTCCGGG
GCAGTTCTTAGGTGGGTCCATCCGTCCTCCACTGGT GACAAACTCCAGA
ACTTCCTGGTTGCTCTTGCTGGGGTATGGCATATATCCAAGAGAAAATAT
TTCCCACAGCAAC ACTCCAAAAGACCATGTATCTGTTTTAGAAGTAAAT
ATTCCTTCCATGAAGGCCTCCGGGGGCATCCACTTCACTG GCAGCATGG
CACAGCCTCCCTTTCTGTAGTAGCTGGCCCTGTAGATATCTCGGGCCATC
CCGAAGTCTCCGATCTT CGCTATCCTCCCAGCTCCCGGACAGGTCAGCA
GACAGTTTCTAGCAGCAATATCGCGGTGGATAAAGTGATTTTCC TCCAG
GTACTGACAGCCACAGGCAATGTCCCGAGCTACGTGCAGAAGATCCAACA
TGGCCAGGGAGGTGGGCTGGT TCGGGCGAGGACGTGTCTCCCTGAGGAA
GGACTTGAGGTCTCCACCAGCCATGAGCTCCAGCAGGATGAAGCGGGG C
AGGGCTTGCAGACTCACCCCGATGCAGCGGACAATGTTCTGGTGGTTGAA
TTTACTGATGATCAGAGCTTCCATG AGGAAGTCCAGCTCATCTTGCTCT
GAACACACTTCCGGCAGCGTCTTTACAGCCACTTGTAGAGGGCTTGGGTC
AT TGGGCATTCCAGATACCTGGCCTTCATACACCTCCCCAAATGCGCCA
TGGCCTAGGCCCCGGATGAGGGTGATGTT TTTCCGAGGCACCTCCTTCA
GGTCACTGATGGATGAAGTCTTGCCAGCAAAGCAGTAGTTGGGGTTGTAG
TCTGTC ATGATGGTCGAGGTCCGTAGCTTGCTCAGCTTATACTCAGGGC
TCTGCAGCTCCATCTGCATAGCTTGCAACTCCT GGTGCTTCCGGCGGTA
CACAATCATGATGCCGGAGAATGCCAGAACGAGAGCGGCCACCAGGGCAG
AGGTCACGAC GGAGAGGATCAATGAGAGCGGCAGGTGGGGTTCCGGGGT
GGGCGACACAATGCAGGAGACACCATCGTCAGCCAGC ACGGTCCCATGG
TCACAGAAGCAGATGACTTTGTGGCTCTCGGGGTCCATGTGACATTCGTC
TACCTCACAGTGGC TGCAGTTTAGATAGTGCTTGATATTCACTTCCCCA
TGGCCCTCCATCACTTTTAAGGCCGGGGTATACAGGATACC CAATGGAC
TGATGAAGGAAACCCCATCTTCCCCATCCATTTCGGGGTCATTGTTTGAT
GCTGCGTTGCCACCTATA TATCCTCCGCCTCCTCCACCTGAGGAGCACC
CCCCTCCACCCCCTCCGAAACCCCCTCTTGTCTCCCACCCCCACT TCTT
CATGGCCTGGGGGCAGGAATGTCCTCCGGCGGCGCCCTCCAACAAAGACT
TTCCGGCCCAGAGCAAGGAAGT GTTATCATTCCAGCCGCCTCCACCACC
TGCGGCTCCGGAATTGCCGTTCAGCCCTAGAACTGAGGAGTTATTCTCC
AGTCTCTCTGGGTGGAACGTTTCTGTCTTGGCCCCATAGGCCCTGCCACC
ACCGCCGGCCGCAATAATCAGGGGCA CGGGCACGCCGTCTTTCATCTTA
AACACGTAGGTGGCTCCACCCCCACCTCCTCCTCCTCCTGCCCACTCGTG
AAC GCTTCTGTTCACTCGGATCTCTTCTTCTATCACATTGTTCTCGCCG
ACACATACTTTCTGGATTAGTTGGTTTGCC CTGGGACAGGCATCTTCCC
CTTGTTGACCGACAAGGATGTAGAGCGTGTCATCTTTCTCCAGATTGAAG
ATGCCCA GGACAGACACGCCATGGGACCGCATCATGGTGTTCTTCCCAC
CTTTTCCGCCAGCTGCTCCATAGCCTGAGATACT GTAGGTGTCAGTAGC
TGGCACTTTCCAAATCTGGATGCCCCTCAAGGGCCCTTCACTTCCCACCA
CCACGCTCAGG TTGGAGTTCTGGTAGGCGTTGTTGCACTGTGCCTGTGT
GGGGCCATGAGGGCCACTGGCCCCACATGTGGTGAACA GCCAGTGAACT
GTAGGGTCGAAGATGGGAGTTGGTCCTGATATGTTTGCCCAGGGTTTTGG
CTCCTTGTTTGGGTT TTTCTCAAACAGATTTCTGGATTTGGGTACTGCA
TTCAGGGACATTTTCTCCTCCCCACTGATGGTGAGGTAGCAG TCGAGGC
TGATGGAAATGTTGTCAAATGCCACGGTTGCCCTGGATCCTGGGCCCCAC
CATGTGACTATTTGCAGCC AGAACCTGTCAGTCACATCGAGGAGCGACA
ACACTGTCCACTGCCACAGGCTTAGGCCTTCATTGGTGGCAACATG CCA
GACGGTCCGGCTTTGCTCCTTTCCAGTTTTGTTCTCCACCAGCACCAAAG
ATACATTTCCTTTCAGAACCCCA CGGATGAGCCAGGACATCCGGAGCTC
ACAAGGAGAGCTCTTCATTGGTGCGGGGAATGTAGCACTGGTCACTGTTG
CACTTTCTGAAGTGGGGTCGTCAGTGGTGCTGAGCAACAGGGCATGGCC
TTGGTGGCCCTGGGAATGGGTATCTTT CAGGGTCTTCACTTGCCATCGG
GGCACACGGGGTGAGAGTGGACTTTGGGTCCAGCCACAGAAGCCATCCTC
GAAG TTACAGTAAAACCCAGCAGGAAGTTTACTGCATAGCTGACCCTCA
TCTTCTCCTTGGGCACAGTTCTGGTGGAAGT CACAGGCTTGCCCGAGCT
GGAGGACTGTCCCGTTCCAACAAGTGAAGGAACTCTGCAAGGCCATCTTG
GAGCCTGG GGATGTTCCTTCACTGCAGTTCTTCAGGGCAAAGAAATCCA
CCGCAGACAAGCTCCGGTTTCCACTCGAGATGTAT TCCAGGGCCACTCG
GAATGGGTTCTCTGGGCGCCCGACTCTCCCATGCAGCACTGTCCAGCCAT
GTTTCCCCGGGG TAGGCACCAGAAGAATCTCTCTTCCAGCTTCATTGTG
GGGTAGGAGCTGGGCAACATATCTCCCAGAAGGCTGTAG ATGTCTGTGC
ACTGAGACAGCCAGCGTGCAGTGCTCACTACTGCTCCTCATCCAGGGGCT
CAGGATGGTATGCTTG GAATCTGCAGAGGTGTTCAGGAGGAGGAAGGAG
CCTCTGGGCATCTCTTTCAAATGCTCTGCCTCTGGCCCATCCA ACAAGT
TCATCCTCGAGGCCTCCTCAGAGGGCACACGGCGCCAGGACCAGCTCTGG
TTCCCATGGGTGTGGAGGGG AGGGGAGTATTCTAGCTCACAGGGAAAAT
CGAAGCTGCACTCCAGACCATATCGACTGCGGTGGGAAAGGAAAGGG AA
GGAGTCTTTCATTGTCCAGGTGAGATTCCATAGAAAGTTACCAGGAGGAG
AAGGCATTTCTGAGGGTGATTCCA AGGAGCTGTGACCGGTCCCGAAGAT
CTGGAAGAGAAGGCGGGGCTGGGAGGCTCGGATCGCAGTGGATAGCCTTC
C CTCCCTGCCCACTTCCGATGCCTTCTTCTCAGGCATCAGGCGGATCCT
CAGCCGCCCTTCGCCGTGTAGAATCCAC CAGCTGAACAGCTCGCTAAGG
TTGAACTGGAGTATCCCCACAGCCGCCGCCTCCTCTGGGGGACCAATGCA
GCCTT CAAGGATCGTCTCCTCGCCCAGCTCCAGCACCAGCTGTTTGGCA
CGCCTGAGCTTGCGCACCGAGCCGCCCTTCAG CACCCTGGACAGCGTCG
GAGCCGCCTCCGGACTAGGAGAACTGGCTCCCTCTGCCCAGGAGATTCCA
GGCGATGGC CCCAGCAACCTGAGCAGAGGGTCGCAGTCCAGAGCCAGTG
ATCCCCGCGCCTCCAGCCCGCCAGCCCCGGGCTCCG AGGGGGACGGTGG
CTGCGGCAGCAGCAGGTCTCGGGCATAGACGCGGAAGAGTGAGGGCACCA
CGAAGTCCACCGC CAGACTCTTCCTCTGCAGGCGCGAATAGCTGAGCGG
CTCCCGGGGCTGCAGAGGCGGCCCTGAGGCTGGAGAACCC GCGCGCTGA
TCGGTTGCAGCTCCGGAGTACGAGGCTGCTGTCAAAAGCAGCGGCGGCAG
CAGCCACAGGAACCCAA GGGCTCCCATCCCTCCGGAGGGCGAGTTTACA
CTAGACTCCCTTCCCTGCCTGGTCCAGCCTCACCTTTCACTCTC CCCTA
AACAGAAAGGGGCTCCAAATAGTCCTTGGTCCTGAACGGCTCCTTCCACT
TGGTCTCAAGGACTGTGTACA CTATAGAGCGCCCTGCTTTCCCCAGTCT
ACAAGGAGGCAAAAAGGGTTCTCAGCAAAACGGATGAGAAAGTCAGGG G
GGAGTTCGGAGACAGTGAGACACCTTGGGTTCAACAGCTGGATCCTGCGC
CTCTCGCCATGCTGCGCCCCATTTG CTCAGGGACCAGAGAAATCACTAT
GGTTGAGGGAGGTGGCAATTAGGTACCGTTCAAGGAGCCCTCGGTGACCA
GA GCTCTCCTCTTCGCCTCCAATCTTCTGCAACTTTCCAGGCTGCGAGC
CGCGGTTCTGAGTCGGTCACTGTCTTGCC CACGTCCTCCGTTCCTGTCA
CCTCCTCTGCGCTCCTTCCCAGGTCTAGGCAAGGACCACCGGAGGGGGCT
GCTTTC TAGCGCCCCCTCAAGTGACCTCCGCTGCAGGAAGTCTTCGGAT
CGATCACCCTGCCCGCTGAACTTCTGGGCGTGA ACGCGAGCTCTGCGTT
GCGGAGAGGAGGTCCCGCTGCCTCCACGCGCACAAGTTTGCAGCGTCCTT
GCTGTCACCG ACGTCTTCGTTCCTCGGAGTTCACTCCCAAGCACCAGAG
CGCCCCGGCCGTGCCGCCGAGTGAAAACATTCGGGGC AGAGGAGCCCGC
AACGTAGCCAGCTCCCTCCTTACCTACCAGCAGCGCCCGCGGTTTTGTTG
ACTGGCCAGGGGGC GAACGGAAAGCGTCTTACTGCCCCGAGAACCCCTG
GACAGAAGAAGCGGGAGGAGCCAGTTGCCACCGCCGCTGCC GCCCCCAG
AGCTGC

We claim:

1. A double stranded ribonucleic acid (dsRNA) agent for inhibiting expression of ALK, wherein the dsRNA agent comprises a sense strand and an antisense strand forming a double stranded region,
wherein the sense strand comprises at least 15 contiguous nucleotides differing by no more than 3 nucleotides from the nucleotide sequence of SEQ ID NO: 1 and the antisense strand comprises at least 15 contiguous nucleotides differing by no more than 3 nucleotides from the nucleotide sequence of SEQ ID NO: 2.

2. A double stranded ribonucleic acid (dsRNA) agent for inhibiting expression of ALK, wherein the dsRNA agent comprises a sense strand and an antisense strand forming a double stranded region,
wherein the antisense strand comprises a region of complementarity to an mRNA encoding ALK, and wherein the region of complementarity comprises at least 15 contiguous nucleotides differing by no more than 3 nucleotides from the nucleotide sequence of SEQ ID NO:2.

3. A double stranded ribonucleic acid (dsRNA) agent for inhibiting expression of ALK, wherein the dsRNA agent comprises a sense strand and an antisense strand forming a double stranded region,
wherein the antisense strand comprises a region of complementarity to an mRNA encoding ALK, and wherein the region of complementarity comprises at least 15 contiguous nucleotides differing by no more than 3 nucleotides from any one of the antisense nucleotide sequences in Tables 2, 3 or 4.

4. The dsRNA agent of any one of claims 1-3, wherein the sense strand comprises at least 15 contiguous nucleotides differing by no more than three nucleotides from any one of the nucleotide sequence of nucleotides 24-44, 57-77, 157-177, 245-265, 286-306, 314-334, 326-346, 343-363, 366-386, 382-402, 430-450, 448-468, 465-485, 499-519, 512-532, 587-607, 599-619, 612-632, 624-644, 636-656, 688-708, 702-722, 714-734, 754-774, 774-794, 798-818, 813-833, 827-847, 847-867, 859-879, 873-893, 919-939, 955-975, 987-1007, 1079-1099, 1100-1120, 1112-1132, 1124-1144, 1136-1156, 1148-1168, 1160-1180, 1183-1203, 1318-1338, 1361-1381, 1373-1393, 1394-1414, 1445-1465, 1462-1482, 1477-1497, 1499-1519, 1511-1531, 1524-1544, 1546-1566, 1560-1580, 1594-1614, 1616-1636, 1629-1649, 1644-1664, 1659-1679, 1672-1692, 1684-1704, 1697-1717, 1709-1729, 1724-1744, 1744-1764, 1787-1807, 1801-1821, 1847-1867, 1879-1899, 1895-1915, 1907-1927, 1930-1950, 1942-1962, 1954-1974, 1972-1992, 1985-2005, 2000-2020, 2023-2043, 2063-2083, 2075-2095, 2094-2114, 2107-2127, 2119-2139, 2138-2158, 2152-2172, 2164-2184, 2176-2196, 2196-2216, 2215-2235, 2246-2266, 2269-2289, 2291-2311, 2314-2334, 2327-2347, 2339-2359, 2354-2374, 2366-2386, 2390-2410, 2402-2422, 2420-2440, 2444-2464, 2464-2484, 2477-2497, 2489-2509, 2503-2523, 2534-2554, 2556-2576, 2575-2595, 2587-2607, 2599-2619, 2612-2632, 2637-2657, 2651-2671, 2672-2692, 2684-2704, 2707-2727, 2720-2740, 2732-2752, 2753-2773, 2773-2793, 2806-2826, 2819-2839, 2831-2851, 2843-2863, 2855-2875, 2875-2895, 2889-2909, 2901-2921, 2914-2934, 2929-2949, 2954-2974, 2980-3000, 2992-3012, 3047-3067, 3064-3084, 3076-3096, 3118-3138, 3130-3150, 3142-3162, 3159-3179, 3179-3199, 3191-3211, 3205-3225, 3218-3238, 3233-3253, 3251-3271, 3263-3283, 3275-3295, 3305-3325, 3317-3337, 3332-3352, 3347-3367, 3365-3385, 3378-3398, 3424-3444, 3436-3456, 3464-3484, 3504-3524, 3526-3546, 3538-3558, 3556-3576, 3570-3590, 3585-3605, 3599-3619, 3611-3631, 3625-3645, 3639-3659, 3651-3671, 3751-3771, 3774-3794, 3818-3838, 3834-3854, 3855-3875, 3895-3915, 3911-3931, 3926-3946, 3946-3966, 3959-3979, 3971-3991, 3991-4011, 4007-4027, 4037-4057, 4052-4072, 4064-4084, 4097-4117, 4109-4129, 4127-4147, 4139-4159, 4151-4171, 4163-4183, 4178-4198, 4198-4218, 4211-4231, 4234-4254, 4246-4266, 4259-4279, 4275-4295, 4337-4357, 4354-4374, 4394-4414, 4416-4436, 4429-4449, 4442-4462, 4459-4479, 4474-4494, 4526-4546, 4558-4578, 4619-4639, 4642-4662, 4656-4676, 4671-4691, 4693-4713, 4705-4725, 4717-4737, 4734-4754, 4747-4767, 4762-4782, 4774-4794, 4792-4812, 4807-4827, 4820-4840, 4847-4867, 4859-4879, 4871-4891, 4886-4906, 4901-4921, 4913-4933, 4948-4968, 4960-4980, 4972-4992, 5000-5020, 5012-5032, 5024-5044, 5046-5066, 5058-5078, 5075-5095, 5092-5112, 5111-5131, 5123-5143, 5135-5155, 5147-5167, 5159-5179, 5173-5193, 5190-5210, 5216-5236, 5229-5249, 5281-5301, 5293-5313, 5307-5327, 5327-5347, 5339-5359, 5378-5398, 5392-5412, 5405-5425, 5428-5448, 5449-5469, 5461-5481, 5477-5497, 5509-5529, 5523-5543, 5559-5579, 5576-5596, 5614-5634, 5633-5653, 5645-5665, 5657-5677, 5671-5691, 5686-5706, 5705-5725, 5723-5743, 5749-5769, 5761-5781, 5773-5793, 5787-5807, 5814-5834, 5826-5846, 5845-5865, 5857-5877, 5869-5889, 5881-5901, 5897-5917, 5909-5929, 5924-5944, 5956-5976, 5972-5992, 5984-6004, 5997-6017, 6032-6052, 6044-6064, 6056-6076, 6079-6099, 6097-6117, 6110-6130, 6128-6148, 6140-6160, 6153-6173, 6183-6203, 6203-6223, 6234-6254, or 6247-6267 of SEQ ID NO: 1, and the antisense strand comprises at least 15 contiguous nucleotides from the corresponding nucleotide sequence of SEQ ID NO: 2.

5. The dsRNA agent of any one of claims 1-4, wherein the antisense strand comprises at least 15 contiguous nucleotides differing by no more than three nucleotides from any one of the antisense strand nucleotide sequences of a duplex selected from the group consisting of AD-1289971, AD-1289972, AD-1289973, AD-1289974, AD-1289975, AD-1289976, AD-1289977, AD-1289978, AD-1289979, AD-1289980, AD-1289981, AD-1289982, AD-1289983, AD-1289984, AD-1289985, AD-1289986, AD-1289987, AD-1289988, AD-1289989, AD-1289990, AD-1289991, AD-1289992, AD-1289993, AD-1289994, AD-1289995, AD-1289996, AD-1289997, AD-1289998, AD-1289999, AD-1290000, AD-1290001, AD-1290002, AD-1290003, AD-1290004, AD-1290005, AD-1290006, AD-1290007, AD-1290008, AD-1290009, AD-1290010, AD-1290011, AD-1290012, AD-1290013, AD-1290014, AD-1290015, AD-1290016, AD-1290017, AD-1290018, AD-1290019, AD-1290020, AD-1290021, AD-1290022, AD-1290023, AD-1290024, AD-1290025, AD-1290026, AD-1290027, AD-1290028, AD-1290029, AD-1290030, AD-1290031, AD-1290032, AD-1290033, AD-1290034, AD-1290035, AD-1290036, AD-1290037, AD-1290038, AD-1290039, AD-1290040, AD-1290041, AD-1290042, AD-1290043, AD-1290044, AD-1290045, AD-1290046, AD-1290047, AD-1290048, AD-1290049, AD-1290050, AD-1290051, AD-1290052, AD-1290053, AD-1290054, AD-1290055, AD-1290056, AD-1290057, AD-1290058, AD-1290059, AD-1290060, AD-1290061, AD-1290062, AD-1290063, AD-1290064, AD-1290065, AD-1290066, AD-1290067, AD-1290068, AD-1290069, AD-1290070, AD-1290071, AD-1290072, AD-1290073, AD-1290074, AD-1290075, AD-1290076, AD-1290077, AD-1290078, AD-1290079, AD-1290080, AD-1290081, AD-1290082, AD-1290083, AD-1290084, AD-1290085, AD-1290086, AD-1290087, AD-1290088, AD-1290089, AD-1290090, AD-1290091, AD-1290092, AD-1290093, AD-1290094, AD-1290095, AD-1290096, AD-1290097, AD-1290098, AD-1290099, AD-1290100, AD-1290101, AD-1290102, AD-1290103, AD-1290104, AD-1290105, AD-1290106, AD-1290107, AD-1290108, AD-1290109, AD-1290110, AD-1290111, AD-1290112, AD-1290113, AD-1290114, AD-1290115, AD-1290116, AD-1290117, AD-1290118, AD-1290119, AD-1290120, AD-1290121, AD-1290122, AD-1290123, AD-1290124, AD-1290125, AD-1290126, AD-1290127, AD-1290128, AD-1290129, AD-1290130, AD-1290131, AD-1290132, AD-1290133, AD-1290134, AD-1290135, AD-1290136, AD-1290137, AD-1290138, AD-1290139, AD-1290140, AD-1290141, AD-1290142, AD-1290143, AD-1290144, AD-1290145, AD-1290146, AD-1290147, AD-1290148, AD-1290149, AD-1290150, AD-1290151, AD-1290152, AD-1290153, AD-1290154, AD-1290155, AD-1290156, AD-1290157, AD-1290158, AD-1290159, AD-1290160, AD-1290161, AD-1290162, AD-1290163, AD-1290164, AD-1290165, AD-1290166, AD-1290167, AD-1290168, AD-1290169, AD-1290170, AD-1290171, AD-1290172, AD-1290173, AD-1290174, AD-1290175, AD-1290176, AD-1290177, AD-1290178, AD-1290179, AD-1290180, AD-1290181, AD-1290182, AD-1290183, AD-1290184, AD-1290185, AD-1290186, AD-1290187, AD-1290188, AD-1290189, AD-1290190, AD-1290191, AD-1290192, AD-1290193, AD-1290194, AD-1290195, AD-1290196, AD-1290197, AD-1290198, AD-1290199, AD-1290200, AD-1290201, AD-1290202, AD-1290203, AD-1290204, AD-1290205, AD-1290206, AD-1290207, AD-1290208, AD-1290209, AD-1290210, AD-1290211, AD-1290212, AD-1290213, AD-1290214, AD-1290215, AD-1290216, AD-1290217, AD-1290218, AD-1290219, AD-1290220, AD-1290221, AD-1290222, AD-1290223, AD-1290224, AD-1290225, AD-1290226, AD-1290227, AD-1290228, AD-1290229, AD-1290230, AD-1290231, AD-1290232, AD-1290233, AD-1290234, AD-1290235, AD-1290236, AD-1290237, AD-1290238, AD-1290239, AD-1290240, AD-1290241, AD-1290242, AD-1290243, AD-1290244, AD-1290245, AD-1290246, AD-1290247, AD-1290248, AD-1290249, AD-1290250, AD-1290251, AD-1290252, AD-1290253, AD-1290254, AD-1290255, AD-1290256, AD-1290257, AD-1290258, AD-1290259, AD-1290260, AD-1290261, AD-1290262, AD-1290263, AD-1290264, AD-1290265, AD-1290266, AD-1290267, AD-1290268, AD-1290269, AD-1290270, AD-1334980, AD-1334981, AD-1334982, AD-1334983, AD-1334984, AD-1334985, AD-1334986, AD-1334987, AD-1334988, AD-1334989, AD-1334990, AD-1334991, AD-1334992, AD-1334993, AD-1334994, AD-1334995, AD-1334996, AD-1334997, AD-1334998, AD-1334999, AD-1335000, AD-1335001, AD-1335002, AD-1335003, AD-1335004, AD-1335005, AD-1335006, AD-1335007, AD-1335008, AD-1335009, AD-1335010, AD-1335011, AD-1335012, AD-1335013, AD-1335014, AD-1335015, AD-1335016, AD-1335017, AD-1335018, AD-1335019, AD-1335020, AD-1335021, AD-1335022, AD-1335023, AD-1335024, AD-1335025, AD-1335026, AD-1335027, AD-1335028, AD-1335029, AD-1335030, AD-1335031, AD-1335032, AD-1335033, AD-1335034, AD-1335035, AD-1335036, AD-1335037, AD-1335038, AD-1335039, AD-1335040, AD-1335041, AD-1335042, AD-1335043, AD-1335044, AD-1335045, AD-1335046, AD-1335047, AD-1335048, AD-1335049, AD-1335050, AD-1335051, AD-1335052, AD-1335053, AD-1335054, AD-1335055, AD-1335056, AD-1335057, AD-1335058, AD-1335059, AD-1335060, AD-1335061, AD-1335062, AD-1335063, AD-1335064, AD-1335065, AD-1335066, AD-1335067, AD-1335068, AD-1335069, AD-1335070, AD-1335071, AD-1335072, AD-1335073, AD-1335074, AD-1335075, AD-1335076, AD-1335077, AD-1335078, AD-1335079, AD-1335080, AD-1335081, AD-1335082, AD-1335083, AD-1335084, AD-1335085, AD-1335086, AD-1335087, AD-1335088, AD-1335089, AD-1335090, AD-1335091, AD-1335092, AD-1335093, AD-1335094, AD-1335095, AD-1335096, AD-1335097, AD-1335098, AD-1335099, AD-1335100, AD-1335101, AD-1335102, AD-1335103, AD-1335104, AD-1335105, AD-1335106, AD-1335107, AD-1335108, AD-1335109, AD-1335110, AD-1335111, AD-1335112, AD-1335113, AD-1335114, AD-1335115, AD-1335116, AD-1335117, AD-1335118, AD-1335119, AD-1335120, AD-1335121, AD-1335122, AD-1335123, AD-1335124, AD-1335125, AD-1335126, AD-1335127, AD-1335128, AD-1335129, AD-1335130, AD-1335131, AD-1335132, AD-1335133, AD-1335134, AD-1335135, AD-1335136, AD-1335137, AD-1335138, AD-1335139, AD-1335140, AD-1335141, AD-1335142, AD-1335143, AD-1335144, AD-1335145, AD-1335146, AD-1335147, AD-1335148, AD-1335149, AD-1335150, AD-1335151, AD-1335152, AD-1335153, AD-1335154, AD-1335155, AD-1335156, AD-1335157, AD-1335158, AD-1335159, AD-1335160, AD-1335161, AD-1335162, AD-1335163, AD-1335164, AD-1335165, AD-1335166, AD-1335167, AD-1335168, AD-1335169, AD-1335170, AD-1335171, AD-1335172, AD-1335173, AD-1335174, AD-1335175, AD-1335176, AD-1335177, AD-1335178, AD-1335179, AD-1335180, AD-1335181, AD-1335182, AD-1335183, AD-1335184, AD-1335185, AD-1335186, AD-1335187, AD-1335188, AD-1335189, AD-1335190, AD-1335191, AD-1335192, AD-1335193, AD-1335194, AD-1335195, AD-1335196, AD-1335197, AD-1335198, AD-1335199, AD-1335200, AD-1335201, AD-1335202, AD-1335203, AD-1335204, AD-1335205, AD-1335206, AD-1335207, AD-1335208, AD-1335209, AD-1335210, AD-1335211, AD-1335212, AD-1335213, AD-1335214, AD-1335215, AD-1335216, AD-1335217, AD-1335218, AD-1335219, AD-1335220, AD-1335221, AD-1335222, AD-1335223, AD-1335224, AD-1335225, AD-1335226, AD-1335227, AD-1335228, AD-1335229, AD-1335230, AD-1335231, AD-1335232, AD-1335233, AD-1335234, AD-1335235, AD-1335236, AD-1335237, AD-1335238, AD-1335239, AD-1335240, AD-1335241, AD-1335242, AD-1335243, AD-1335244, AD-1335245, AD-1335246, AD-1335247, AD-1335248, AD-1335249, AD-1335250, AD-1335251, AD-1335252, AD-1335253, AD-1335254, AD-1335255, AD-1335256, AD-1335257, AD-1335258, AD-1335259, AD-1335260, AD-1335261, AD-1335262, AD-1335263, AD-1335264, AD-1335265, AD-1335266, AD-1335267, AD-1335268, AD-1335269, AD-1335270, AD-1335271, AD-1335272, AD-1335273, AD-1335274, AD-1335275, AD-1335276, AD-1335277, AD-1335278, and AD-1335279.

6. The dsRNA agent of claim 1 or 2, wherein the nucleotide sequence of the sense and antisense strand comprise any one of the sense and antisense strand nucleotide sequences in Tables 2, 3, or 4.

7. The dsRNA agent of any one of claims 1-6, wherein the sense strand, the antisense strand, or both the sense strand and the antisense strand is conjugated to one or more lipophilic moieties.

8. The dsRNA agent of claim 7, wherein the lipophilic moiety is conjugated to one or more internal positions in the double stranded region of the dsRNA agent.

9. The dsRNA agent of claim 7 or 8, wherein the lipophilic moiety is conjugated via a linker or carrier.

10. The dsRNA agent of any one of claims 7-9, wherein lipophilicity of the lipophilic moiety, measured by logKow, exceeds 0.

11. The dsRNA agent of any one of claims 1-10, wherein the hydrophobicity of the double-stranded RNA agent, measured by the unbound fraction in a plasma protein binding assay of the double-stranded RNA agent, exceeds 0.2.

12. The dsRNA agent of claim 11, wherein the plasma protein binding assay is an electrophoretic mobility shift assay using human serum albumin protein.

13. The dsRNA agent of any one of claims 1-12, wherein the dsRNA agent comprises at least one modified nucleotide.

14. The dsRNA agent of claim 13, wherein no more than five of the sense strand nucleotides and no more than five of the nucleotides of the antisense strand are unmodified nucleotides.

15. The dsRNA agent of claim 13, wherein all of the nucleotides of the sense strand and all of the nucleotides of the antisense strand are modified nucleotides.

16. The dsRNA agent of any one of claims 13-15, wherein at least one of the modified nucleotides is selected from the group a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a 5′-phosphorothioate group, a nucleotide comprising a 5′-methylphosphonate group, a nucleotide comprising a 5′ phosphate or 5′ phosphate mimic, a nucleotide comprising vinyl phosphonate, a nucleotide comprising adenosine-glycol nucleic acid (GNA), a nucleotide comprising thymidine-glycol nucleic acid (GNA) S-Isomer, a nucleotide comprising 2-hydroxymethyl-tetrahydrofurane-5-phosphate, a nucleotide comprising 2′-deoxythymidine-3′phosphate, a nucleotide comprising 2′-deoxyguanosine-3′-phosphate, and a terminal nucleotide linked to a cholesteryl derivative and a dodecanoic acid bisdecylamide group; and combinations thereof.

17. The dsRNA agent of claim 16, wherein the modified nucleotide is selected from the group consisting of a 2′-deoxy-2′-fluoro modified nucleotide, a 2′-deoxy-modified nucleotide, 3′-terminal deoxy-thymine nucleotides (dT), a locked nucleotide, an abasic nucleotide, a 2′-amino-modified nucleotide, a 2′-alkyl-modified nucleotide, a morpholino nucleotide, a phosphoramidate, and a non-natural base comprising nucleotide.

18. The dsRNA agent of claim 16, wherein the modified nucleotide comprises a short sequence of 3′-terminal deoxy-thymine nucleotides (dT).

19. The dsRNA agent of claim 16, wherein the modifications on the nucleotides are 2′-O-methyl, GNA and 2′fluoro modifications.

20. The dsRNA agent of any one of claims 1-19, further comprising at least one phosphorothioate internucleotide linkage.

21. The dsRNA agent of claim 20, wherein the dsRNA agent comprises 6-8 phosphorothioate internucleotide linkages.

22. The dsRNA agent of any one of claims 1-21, wherein each strand is no more than 30 nucleotides in length.

23. The dsRNA agent of any one of claims 1-22, wherein at least one strand comprises a 3′ overhang of at least 1 nucleotide.

24. The dsRNA agent of any one of claims 1-23, wherein at least one strand comprises a 3′ overhang of at least 2 nucleotides.

25. The dsRNA agent of any one of claims 1-24, wherein the double stranded region is 15-30 nucleotide pairs in length.

26. The dsRNA agent of claim 25, wherein the double stranded region is 17-23 nucleotide pairs in length.

27. The dsRNA agent of claim 25, wherein the double stranded region is 17-25 nucleotide pairs in length.

28. The dsRNA agent of claim 25, wherein the double stranded region is 23-27 nucleotide pairs in length.

29. The dsRNA agent of claim 25, wherein the double stranded region is 19-21 nucleotide pairs in length.

30. The dsRNA agent of claim 25, wherein the double stranded region is 21-23 nucleotide pairs in length.

31. The dsRNA agent of any one of claims 1-30, wherein each strand has 19-30 nucleotides.

32. The dsRNA agent of any one of claims 1-30, wherein each strand has 19-23 nucleotides.

33. The dsRNA agent of any one of claims 1-30, wherein each strand has 21-23 nucleotides.

34. The dsRNA agent of any one of claims 8-33, wherein one or more lipophilic moieties are conjugated to one or more internal positions on at least one strand.

35. The dsRNA agent of claim 34, wherein the one or more lipophilic moieties are conjugated to one or more internal positions on at least one strand via a linker or carrier.

36. The dsRNA agent of claim 35, wherein the internal positions include all positions except the terminal two positions from each end of the at least one strand.

37. The dsRNA agent of claim 35, wherein the internal positions include all positions except the terminal three positions from each end of the at least one strand.

38. The dsRNA agent of claim 35-37, wherein the internal positions exclude a cleavage site region of the sense strand.

39. The dsRNA agent of claim 38, wherein the internal positions include all positions except positions 9-12, counting from the 5′-end of the sense strand.

40. The dsRNA agent of claim 38, wherein the internal positions include all positions except positions 11-13, counting from the 3′-end of the sense strand.

41. The dsRNA agent of claim 35-37, wherein the internal positions exclude a cleavage site region of the antisense strand.

42. The dsRNA agent of claim 41, wherein the internal positions include all positions except positions 12-14, counting from the 5′-end of the antisense strand.

43. The dsRNA agent of claim 35-37, wherein the internal positions include all positions except positions 11-13 on the sense strand, counting from the 3′-end, and positions 12-14 on the antisense strand, counting from the 5′-end.

44. The dsRNA agent of any one of claims 8-43, wherein the one or more lipophilic moieties are conjugated to one or more of the internal positions selected from the group consisting of positions 4-8 and 13-18 on the sense strand, and positions 6-10 and 15-18 on the antisense strand, counting from the 5′end of each strand.

45. The dsRNA agent of claim 44, wherein the one or more lipophilic moieties are conjugated to one or more of the internal positions selected from the group consisting of positions 5, 6, 7, 15, and 17 on the sense strand, and positions 15 and 17 on the antisense strand, counting from the 5′-end of each strand.

46. The dsRNA agent of claim 8, wherein the internal positions in the double stranded region exclude a cleavage site region of the sense strand.

47. The dsRNA agent of any one of claims 7-46, wherein the sense strand is 21 nucleotides in length, the antisense strand is 23 nucleotides in length, and the lipophilic moiety is conjugated to position 21, position 20, position 15, position 1, position 7, position 6, or position 2 of the sense strand or position 16 of the antisense strand.

48. The dsRNA agent of claim 47, wherein the lipophilic moiety is conjugated to position 21, position 20, position 15, position 1, or position 7 of the sense strand.

49. The dsRNA agent of claim 47, wherein the lipophilic moiety is conjugated to position 21, position 20, or position 15 of the sense strand.

50. The dsRNA agent of claim 47, wherein the lipophilic moiety is conjugated to position 20 or position 15 of the sense strand.

51. The dsRNA agent of claim 47, wherein the lipophilic moiety is conjugated to position 16 of the antisense strand.

52. The dsRNA agent of any one of claims 7-51, wherein the lipophilic moiety is an aliphatic, alicyclic, or polyalicyclic compound.

53. The dsRNA agent of claim 52, wherein the lipophilic moiety is selected from the group consisting of lipid, cholesterol, retinoic acid, cholic acid, adamantane acetic acid, 1-pyrene butyric acid, dihydrotestosterone, 1,3-bis-O(hexadecyl)glycerol, geranyloxyhexyanol, hexadecylglycerol, borneol, menthol, 1,3-propanediol, heptadecyl group, palmitic acid, myristic acid, O3-(oleoyl) lithocholic acid, O3-(oleoyl)cholenic acid, dimethoxytrityl, or phenoxazine.

54. The dsRNA agent of claim 52, wherein the lipophilic moiety contains a saturated or unsaturated C4-C30 hydrocarbon chain, and an optional functional group selected from the group consisting of hydroxyl, amine, carboxylic acid, sulfonate, phosphate, thiol, azide, and alkyne.

55. The dsRNA agent of claim 54, wherein the lipophilic moiety contains a saturated or unsaturated C6-C18 hydrocarbon chain.

56. The dsRNA agent of claim 54, wherein the lipophilic moiety contains a saturated or unsaturated C16 hydrocarbon chain.

57. The dsRNA agent of claim 56, wherein the saturated or unsaturated C16 hydrocarbon chain is conjugated to position 6, counting from the 5′-end of the strand.

58. The dsRNA agent of any one of claims 7-57, wherein the lipophilic moiety is conjugated via a carrier that replaces one or more nucleotide(s) in the internal position(s) or the double stranded region.

59. The dsRNA agent of claim 58, wherein the carrier is a cyclic group selected from the group consisting of pyrrolidinyl, pyrazolinyl, pyrazolidinyl, imidazolinyl, imidazolidinyl, piperidinyl, piperazinyl, [1,3] dioxolanyl, oxazolidinyl, isoxazolidinyl, morpholinyl, thiazolidinyl, isothiazolidinyl, quinoxalinyl, pyridazinonyl, tetrahydrofuranyl, and decalinyl; or is an acyclic moiety based on a serinol backbone or a diethanolamine backbone.

60. The dsRNA agent of any one of claims 7-57, wherein the lipophilic moiety is conjugated to the double-stranded iRNA agent via a linker containing an ether, thioether, urea, carbonate, amine, amide, maleimide-thioether, disulfide, phosphodiester, sulfonamide linkage, a product of a click reaction, or carbamate.

61. The double-stranded iRNA agent of any one of claims 7-60, wherein the lipophilic moiety is conjugated to a nucleobase, sugar moiety, or internucleosidic linkage.

62. The dsRNA agent of any one of claims 7-61, wherein the lipophilic moiety or targeting ligand is conjugated via a bio-cleavable linker selected from the group consisting of DNA, RNA, disulfide, amide, functionalized monosaccharides or oligosaccharides of galactosamine, glucosamine, glucose, galactose, mannose, and combinations thereof.

63. The dsRNA agent of any one of claims 7-62, wherein the 3′ end of the sense strand is protected via an end cap which is a cyclic group having an amine, said cyclic group being selected from the group consisting of pyrrolidinyl, pyrazolinyl, pyrazolidinyl, imidazolinyl, imidazolidinyl, piperidinyl, piperazinyl, [1,3] dioxolanyl, oxazolidinyl, isoxazolidinyl, morpholinyl, thiazolidinyl, isothiazolidinyl, quinoxalinyl, pyridazinonyl, tetrahydrofuranyl, and decalinyl.

64. The dsRNA agent of any one of claims 7-61, further comprising a targeting ligand that targets a neuronal cell.

65. The dsRNA agent of any one of claims 7-61, wherein the targeting ligand is a GalNAc conjugate.

66. The dsRNA agent of any one of claims 1-65 further comprising
a terminal, chiral modification occurring at the first internucleotide linkage at the 3′ end of the antisense strand, having the linkage phosphorus atom in Sp configuration,
a terminal, chiral modification occurring at the first internucleotide linkage at the 5′ end of the antisense strand, having the linkage phosphorus atom in Rp configuration, and
a terminal, chiral modification occurring at the first internucleotide linkage at the 5′ end of the sense strand, having the linkage phosphorus atom in either Rp configuration or Sp configuration.

67. The dsRNA agent of any one of claims 1-65 further comprising
a terminal, chiral modification occurring at the first and second internucleotide linkages at the 3′ end of the antisense strand, having the linkage phosphorus atom in Sp configuration,
a terminal, chiral modification occurring at the first internucleotide linkage at the 5′ end of the antisense strand, having the linkage phosphorus atom in Rp configuration, and
a terminal, chiral modification occurring at the first internucleotide linkage at the 5′ end of the sense strand, having the linkage phosphorus atom in either Rp or Sp configuration.

68. The dsRNA agent of any one of claims 1-65 further comprising
a terminal, chiral modification occurring at the first, second and third internucleotide linkages at the 3′ end of the antisense strand, having the linkage phosphorus atom in Sp configuration,
a terminal, chiral modification occurring at the first internucleotide linkage at the 5′ end of the antisense strand, having the linkage phosphorus atom in Rp configuration, and
a terminal, chiral modification occurring at the first internucleotide linkage at the 5′ end of the sense strand, having the linkage phosphorus atom in either Rp or Sp configuration.

69. The dsRNA agent of any one of claims 1-65 further comprising
a terminal, chiral modification occurring at the first, and second internucleotide linkages at the 3′ end of the antisense strand, having the linkage phosphorus atom in Sp configuration,
a terminal, chiral modification occurring at the third internucleotide linkages at the 3′ end of the antisense strand, having the linkage phosphorus atom in Rp configuration,
a terminal, chiral modification occurring at the first internucleotide linkage at the 5′ end of the antisense strand, having the linkage phosphorus atom in Rp configuration, and
a terminal, chiral modification occurring at the first internucleotide linkage at the 5′ end of the sense strand, having the linkage phosphorus atom in either Rp or Sp configuration.

70. The dsRNA agent of any one of claims 1-65 further comprising
a terminal, chiral modification occurring at the first, and second internucleotide linkages at the 3′ end of the antisense strand, having the linkage phosphorus atom in Sp configuration,
a terminal, chiral modification occurring at the first, and second internucleotide linkages at the 5′ end of the antisense strand, having the linkage phosphorus atom in Rp configuration, and
a terminal, chiral modification occurring at the first internucleotide linkage at the 5′ end of the sense strand, having the linkage phosphorus atom in either Rp or Sp configuration.

71. The dsRNA agent of any one of claims 1-70, further comprising a phosphate or phosphate mimic at the 5′-end of the antisense strand.

72. The dsRNA agent of claim 71, wherein the phosphate mimic is a 5′-vinyl phosphonate (VP).

73. The dsRNA agent of any one of claims 1-70, wherein the base pair at the 1 position of the 5′-end of the antisense strand of the duplex is an AU base pair.

74. The dsRNA agent of any one of claims 1-70, wherein the sense strand has a total of 21 nucleotides and the antisense strand has a total of 23 nucleotides.

75. A cell containing the dsRNA agent of any one of claims 1-74.

76. A pharmaceutical composition for inhibiting expression of a gene encoding ALK, comprising the dsRNA agent of any one of claims 1-74.

77. A pharmaceutical composition comprising the dsRNA agent of any one of claims 1-74 and a lipid formulation.

78. The pharmaceutical composition of claim 76 or 77, wherein dsRNA agent is in an unbuffered solution.

79. The pharmaceutical composition of claim 78, wherein the unbuffered solution is saline or water.

80. The pharmaceutical composition of claim 76 or 77, wherein said dsRNA agent is in a buffer solution.

81. The pharmaceutical composition of claim 80, wherein the buffer solution comprises acetate, citrate, prolamine, carbonate, or phosphate or any combination thereof.

82. The pharmaceutical composition of claim 80, wherein the buffer solution is phosphate buffered saline (PBS).

83. A method of inhibiting expression of an ALK gene in a cell, the method comprising contacting the cell with the dsRNA agent of any one of claims 1-76, or the pharmaceutical composition of any one of claims 76-82, thereby inhibiting expression of the ALK gene in the cell.

84. The method of claim 83, wherein the cell is within a subject.

85. The method of claim 84, wherein the subject is a human.

86. The method of claim 85, wherein the subject has an ALK-associated disorder.

87. The method of claim 86, wherein the ALK-associated disorder is type 2 diabetes.

88. The method of claim 86, wherein the ALK-associated disorder is obesity or an obesity associated-disorder.

89. The method of claim 86 or 88, wherein the subject is overweight.

90. The method of claim 86 or 88, wherein the subject is in need or desire of weight loss.

91. The method of claim 86 or 88, wherein the subject is in need or desire of weight maintenance.

92. The method of claim 88, wherein the obesity-associated disorder is selected from the group of disorders consisting of: type 2 diabetes, hypertension, dyslipidemia, atherosclerosis, coronary heart disease, stroke, metabolic syndrome, gallbladder disease, fatty liver, osteoarthritis, sleep apnea, breathing problems, various types of cancer (e.g., endometrial cancer, esophageal adenocarcinoma, gastric cardia cancer, liver cancer, kidney cancer, pancreatic cancer), mental illness (e.g., depression, anxiety), body pain, and difficulty with physical functioning.

93. The method of any one of claims 83-92, wherein contacting the cell with the dsRNA agent inhibits the expression of ALK by at least 30%.

94. The method of any one of claims 83-93, wherein inhibiting expression of ALK decreases ALK protein level in serum of the subject by at least 30%.

95. A method of treating a subject having a disorder that would benefit from reduction in ALK expression, comprising administering to the subject a therapeutically effective amount of the dsRNA agent of any one of claims 1-74, or the pharmaceutical composition of any one of claims 76-82, thereby treating the subject having the disorder that would benefit from reduction in ALK expression.

96. A method of preventing at least one symptom or sign in a subject having a disorder that would benefit from reduction in ALK expression, comprising administering to the subject a prophylactically effective amount of the dsRNA agent of any one of claims 1-74, or the pharmaceutical composition of any one of claims 78-84, thereby preventing at least one symptom or sign in the subject having the disorder that would benefit from reduction in ALK expression.

97. The method of claim 95 or 96, wherein the disorder is an ALK-associated disorder.

98. The method of claim 97, wherein the ALK-associated disorder is type 2 diabetes.

99. The method of claim 97, wherein the ALK-associated disorder is obesity or an obesity-associated disorder.

100. The method of claim 99, wherein the obesity-associated disorder is selected from the group consisting of type 2 diabetes, hypertension, dyslipidemia, atherosclerosis, coronary heart disease, stroke, metabolic syndrome, gallbladder disease, fatty liver, osteoarthritis, sleep apnea, breathing problems, various types of cancer (e.g., endometrial cancer, esophageal adenocarcinoma, gastric cardia cancer, liver cancer, kidney cancer, pancreatic cancer), mental illness (e.g., depression, anxiety), body pain, and difficulty with physical functioning.

101. The method of any one of claims 95-100, wherein the subject is human.

102. The method of any one of claims 95-101, wherein the administration of the agent to the subject causes a decrease in body weight.

103. The method of any one of claims 95-101, wherein the administration of the agent to the subject causes a decrease in waist circumference.

104. The method of any one of claims 95-101, wherein the administration of the agent to the subject causes a decrease in hip circumference.

105. The method of any one of claims 95-101, wherein the administration of the agent to the subject causes a decrease in fat deposition.

106. The method of any one of claims 95-101, wherein the administration of the agent to the subject causes a decrease in triglyceride levels.

107. The method of any one of claims 95-101, wherein the administration of the agent to the subject causes an improvement in blood lipid profile.

108. The method of any one of claims 95-101, wherein the administration of the agent to the subject causes an improvement in blood glucose levels.

109. The method of any one of claims 95-108, wherein the dsRNA agent is administered to the subject at a dose of about 0.01 mg/kg to about 50 mg/kg.

110. The method of any one of claims 95-109, wherein the dsRNA agent is administered to the subject intrathecally.

111. The method of any one of claims 95-110, further comprising determining the level of ALK in a sample(s) from the subject.

112. The method of claim 111, wherein the level of ALK in the subject sample(s) is an ALK protein level in a blood, serum, or cerebrospinal fluid sample(s).

113. The method of any one of claims 95-112, further comprising administering to the subject an additional therapeutic agent.

114. A kit comprising the dsRNA agent of any one of claims 1-74 or the pharmaceutical composition of any one of claims 76-82.

115. A vial comprising the dsRNA agent of any one of claims 1-74 or the pharmaceutical composition of any one of claims 76-82.

116. A syringe comprising the dsRNA agent of any one of claims 1-74 or the pharmaceutical composition of any one of claims 76-82.

117. An intrathecal pump comprising the dsRNA agent of any one of claims 1-74 or the pharmaceutical composition of any one of claims 76-82.


SEQUENCE LISTING (1813 sequences)

SEQ ID NO: 1 - DNA, 6267 bp, Homo sapiens
agctgcaagtggcgggcgcccaggcagatgcgatccagcggctctgggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaaaa

SEQ ID NO: 2 - DNA, 6267 bp, Homo sapiens
ttttagtcattacaaataactcctttatttccgttccctctcccctcaaatggctcatgtccacatcaacaaggcaaggaaacatctatgaccccaactatgaaacatagaagcagctaattctgactacattgaagcagagcacacacaatttgaaagaagcataaggaagtatacaacaaacatgcataaaaaccttatgcaaccacatctgggccttgtatttatcactcatttttatgatattttcttctttcgaaagaataggatgaacccatgctcaaaacctttctaaagcattttcaaaatacagcttttttggtggtacttcaaaataggttggcacaaaacaaaacgtgacatttggtctctggtttgtgaaggagccattgcctctctctcctccacggtcttagggatcccaaggaagagaagtgagtgtgcgaccgagctcagggcccaggctggttcatgctattcttgcttttcagaatggtatcctcgtaatgaccagctccaggggcagtagcggcttctaagggcaagccctgttgctggtagccgtaattgacattcccacaagggaagtgacgtagcctgaacagaggtacctccttcatattggcagtcagcgaagagggctctaggagcagtgaggcccccggaagtctcccagttgcaacgttaggtgggacagtacagcttccctccagccccaggttacccctgtcgtgtggctccttctttgctataggattattctttttggtgggtttctctgtaaaccaggagccgtacgttgggttccacaagctggtgggcttgtttctggatccgtggaccttgtgcaactccgaaggagggttggactgagagaatgccatattcacgtgtcccccttccacggccggccctctagggactcgaacagagatctctgcagctgtgggtttctttgcagccttgccagaggaggtggtaggcagaggtggtggggcagctgggctgcgctcctcctcccgttttgcctgttgagagaccaggagaggaggaaccccctcagggtccttgggcctcacaggcactttctcttcctcttccacaagtggaccatattctatcggcaaagcggtgttgattacatccgggtcctgggtgcagtattcaatcctctccaaaatgatggcaaagttgggcctgtcttcaggctgatgttgccagcactgagtcattatccggtatacaggcccagggcagttcttgggtgggtccatccggcctccactggtgacaaactccagaacttcctggttgcttttgctggggtatggcatatatccaagagaaaagatttcccatagcagcactccaaaggaccatgtgtctgttttagaagtgaatattccttccatgaaggcctctgggggcatccacttaactggcagcatggcacagcctccctttctatagtagctcgccctgtagatgtctcgggccatcccgaagtctccaatcttggccactcttccagggcctggacaggtcaagaggcagtttctggcagcaatgtctcggtggatgaagtggttttcctccaaatactgacagccacaggcaatgtcccgagccacgtgcagaaggtccagcatggccagggaggagggctggctcgggcgagggcgggtctctcggaggaaggacttgaggtctccccccgccatgagctccagcaggatgaaccggggcagggattgcaggctcaccccaatgcagcgaacaatgttctggtggttgaatttgctgatgatcagggcttccatgaggaaatccagttcgtcctgttcagagcacacttcaggcagcgtcttcacagccacttgcagggggcttgggtcgttgggcattccggacacctggccttcatacacctccccaaaggcgccatggcccagaccccgaatgagggtgatgtttttccgcggcacctccttcaggtcactgatggaggaggtcttgccagcaaagcagtagttggggttgtagtcggtcatgatggtcgaggtgcggagcttgctcagcttgtactcagggctctgcagctccatctgcatggcttgcagctcctggtgcttccggcggtacacaatcatgatgccggagaaagccaggaccagggcggccacgagggcagaggtcaccacagagaggatcagcgagagtggcaggtgtggctccggggtgggtgacacaatgcaggagacgccatcctcagccagcaccgtcccgtggtcacagaagcagatgaccttgtggctttcagggtccatgtgacattcgtctacctcacagtgactgcagtttagataatgcttaatattcacttccccgtggccttccatcacttttaaagctggggtgtacaggatgcccagtggactgatgaaggaaaccccatcttccccatccatttcggggtcattgtttgaggctgcattgccgcctatatatcctccgcctcctccacctgaggagcacccccctccaccccctccgaaaccccctcttgtctcccacccccacttcttcatggcctgggggcaggaatgtcctccggtggcaccctcctgcaaagattttccggcccagagcaaggaagtgttatcattccagccacctccaccacctgcggctccggaattgccgtttagccctagaaccgaggagttattctccagtctctctgggtggaacgtgtctgtcttggccccgtaggccctgccaccacctccggctgcaatgatcaggggcaccggcactccatccttcatcttaaatacgtaggtggctccaccccctcctcctccgcctcctgcccactcatgcacgcttctgttcacacggatttcttcttctatcacattgttctctccaatgcagactttctggattaactggtttgtactggggcaggcgtcctctccctgctgcccaaccaggatgtacagcatgtcatccttctccaggttgaagatgcccagcacagacacgccgtgggaccgcatcatggtgttcttcccgcctttcccgccagcagctccgtagcccgagatgctgtaggtgtcggtggctggcaccttccagatctggatgcctttcagggggccctcgctccccacctccacgctcaggttggagttctggtaggcgttgttgcactgtgcctgggtggggccatggggcccgctggccccacatgtggtgaacagccaatgaactgtagggtcaaagatgggggtctgtcttggtgaattttccccgggtttcagctccttgtttgggtttctctcaaacaggtttcttgatttgggtgctgtattctgcaggatcttgtcctctccgctaatggtgaggtagcagtccaggctgatggagatattgtcaaaagccacgatggctctggatccttgtccccaccatgcgaccatctgcagccagaacctgtcagacacatcgaggagaggcaacaccatccactgccacaggctcaagccttcataggcggcgacatgccagaccatcctgccttgctccttcccggttttgttctccactagcaccaaggacacgtttcccctcaagactccacgaatgagccaggacattcggagctcacatggagagctcttgatcggtgcaggaaacgtagcactggtcactgtagcactttcagaagcggggacatcagtggtactgagcaatagagcatggtcttggtggtcctggaaccgggcatcctttagggtcctgacctgccattgaggagtgtggggtgacagtgtgccttgggtccagccacagaagccatcttcaaagttgcagtaaaaacccacaggcagtttccggcacatctggctctcatcttctccctgggcacagtcctggtggaagtcacaggcctgcccaagctggaggactgtcccattccaacaagtgaaggagctctgcagggccatcttggagcctggggatgttccttcactgcagttcttcagggcaaagaagtccactgcagacaagctgcggtttccactggagatgtattccagggccactcgaaatgggttgtctggacgcccgattcttccctggagcactgtccaaccatgcttccctggagtgggcatcaggaggatctctcttgcagcctcgttgtggggcagcagctgggcaatgtaccttccagagggctgcaggtgcctgtgcaccgagacggccagtgtgcagtgctcactgctgctcctcatccacggactcaggatggtgtgcttggagtcagctgaggtgttgagaaggagaaaggagcctctgggcatctccttagaacgctctgccccaggcccatccagcaagtccatctgggaggcctcctcggaggggatgcggcgccaggaccagctctggttcctgaggtcatgcagtggaggggaatactccagctcacaggggaagtcaaagctgcactccagaccatatcggctgcgatgagacaggaaagggaaggagtctttcattatccaggtgagattccatgtaaaataatcaggagaaggagaaggcatgtttgttggtgattccaaggagctatgaccagtcccgaagatctggaagagaaggcggggctgggaggcgcgaattgccgcggacagccttccctctctgcccacttccgacgccttcttctcgggcatcaggcggatcctcagtcgcccttcgccttggcgaatccaccaactgaacagctcgctgagattgaactggagcagccccacagccgcctccccggggggcccgacgcaaccctccaagatcgcctcctcgcccagctccagcaccaactgcttggcacgccggagcttgcgcacggagccgcccttcagcaccctggacagcgtccgggcctctgccggggctggtgaaccggcggtccaggagacccccggcgccggccccagcaacctgagcagcggggcgcagtccagagctagcgagccgcgggcctcgggcctgccagccttcagctccgaggaggatggtggcagcagtaggtcccgggcgtagacacggaagagcgagggcaccacgaagtcaactgccagactcttcctctgcaggcgcgagtagctgagtggctcccggggctgcagcggcggccccgcagctggggagcccgcgcgctggccggtccccatcccggagcccacagctgccgtggaaagcagcagcggcaggagccacaggagcccgatggctcccatcccgccggaggaggccgtttacactgctctccgggcccagcctcacccttcgctctccccgagatgggaagaggctctgaacagtccttggtacccagcggctccttccacctgatctccagaggactgtgcgtgcgcgcaagtctcttgctttcccccaactgcacggaggcgagcaggagtctaaatgaaacagacctggaagctcaggggcgagtccagagacactcaagcacactgggctcactggctgggaccttgagcctcccgctctccgcgccgagtgccgcgcccccgtctgtagctcgctgcgctcggtacagaggaactactatggttgaagggaggtggcagttgggtaccgtcctctcctgccccccgcagtcggagctggggtctgtcccctctcggggcagcctccaatctctgcaacttttaaggctgagaacggcggctcccagctgctgcacgctgtcctggccgccttttgcgttccttttggctcctccaagctcttctgcccggtctgggcgggaaccgagggcggaggctgccgtcttgcgcaccctcaagctatctctccgctgcgggaaggcttcggactgtctgcctgctgaacttctgggcgtgaatcccagcccccgcgctgcgcaagtttgcagcgtccttgctctcaccggcgcctcggctcctcagagttcgcaggcactggagcggccccggcggcagcagctgagggcgttcagggcaggggcgcccgaaatcttgccgccccctcctcacccatcatcagcgcccgcggctttgggtggccgaccagagggcggccggaaagcacctcggtgccccgcgaccctccgaacagaggcggcgggaggtaccagctgctaccaccgctgccgcccccagagccgctggatcgcatctgcctgggcgcccgccacttgcagct

SEQ ID NO: 3 - DNA, 7385 bp, Macaca fascicularis
agctgcaagtggcgggcgcccaggcagatgcgatccagcagctctgggggcggcagcggcggtagcagctggtacctcccgccgcctctgttcggagggtcgcggggcaccgatgtgctttccggccgccctctggccggccacccaaagccgcgggcgctgataatgggtgaggaggggtcggcaagatttcgggagcccctgccctgaacgccctcagctgctgccgcctgcgaactctgaggaaccgagcgaggcgcgggcgacagcaaggacgctgcaaacttgcgcagcgcgggggttcggattcacgccctagaagttcagcgggcagagcagtccgaagccttcccgcagcggagagatagcttgagggtgggcgagaaggcagcccccgtcgtcggatcccacccagaccgggcagaggagctcggaggagccaaaaggaacgcaggaggcggccaggacagcgtgcagcagctgggagccgccgttctcagtctggaaagttgcagagattggaggctgccccgagaggggacagaccccagctccgaccgcggggggcaggagaggacggtacccaactgccacctcccttcaaccatagtagttcctctgtgcggagcgcagcgagctactgacgggggagcggcactcggcgtggcgagcgggagactcaaggtcccagtcagtgagcccagcgtgcttgagggtctctggactcgcccctgagattacagctgtgtttccttgagactcctgctcgcctccgtgcagttgggggaaagcaagagacttgcgctcacgcacagtcctctggagatcaggtggaaggagccgctgggtaccgaggactgttcagagcttcttcccatctaggggagagcgaagggcgaggctgggcctggagagcagtgtaaacggcctccttcggcgggatgggaaccatcgggctcctgtggctgctgccgctgctgctttccactgcagctttgggctctgggacggggaccggccagcgcacgggatccccagctgcggggccgccgctgcagccccgggagccgctcagctactctcgcctgcagaggaagagtctggcagtggacttcgtagtgccctcactcttccgtgtctacgcccgggacctgctgctgccgccatccccctcggagctgaaggctggcaggcccgaggcgcgaggctcgctcgctctggactgcgccccgctgctcaggttgctggggccggcgcccggggtctcctggacagccggctcacccaccccggcagaggcccggacgctgtccagggtgctgaagggcggctcggtgcgcaagctccggcgtgccaagcagctggtgctggagctgggcgaggaggcgatcttggagggttgcgtcgggccccctggggaggcggctgtggggctgctccagttcaacctcagcgagctgttcagttggtggattcgccagggcgaagggcgactgaggatccgcctgatgcccgagaagaaggcgtcggaagtgggcagagagggaaggctgtccgcggcaatccgcgcctcccagccccgccttctcttccagatcttcgggactggtcatagctccttggagtcaccaacaaacataccttctccttctcctgattattttacatggaatctcacctggataatgaaagactccttccctttcctgtctcatcgcagccgatatggtctggagtgcagctttgacttcccctgtgagctggaatattcccctccactgcacgacctcaggaaccagagctggtcctggcgccgtgttccctccgaggaggcctcccagatggacttgctggatgggcctgaggcagagcgttctaaggagatgcccagagggtcctttctcctcctcaacacctcagctgactccaagcacaccatcctaagtccgtggatgaggagcagcagtgagcactgtacgctggccgtctcagtgcacaggcacctgcagccctctggaaggtacattgcccagctgctgccccacaacgaggctgcaagagagatcctcctgatgcccaccccagggaagcatggttggacagtgctccagggaagaatcgggcgcccagacaacccctttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgcgcccagggagaagacgagagccagatatgccggaaactccctgtgggtttttactgcaacttcgaagatggcttctgtggctggacccaaggcacactctcacctcacactcctcagtggcaggtcaggactctaaaggatgcccggttccagaaccaccaagaccatgctctattgctcagtaccactgatgcccccacttcagaaagtgctacagtgaccagtgctacgtttcccgcaccgatcaagagctctccatgtgagctccgaatgtcctggctcattcgtggagtcctgaggggaaacgtgtccttggtgctagtggagaacaaaactgggaaggagcaaggcaggatggtctggcatgtcgccgcctatgaaggcttgagcctgtggcagtggacggtgttgcctctcctcgacgtgtctgacaggttctggctgcagatggttgcatggtggggacaaggatccagagccatcgtggcttttgacaatatctccatcagcctggactgctacctcaccattagtggagaggacaagatcctacagaatacagcacccaaatcaagaaacctgtttgagagaaacccaaacaaggagctgaaacccggggaaaattcaccacgacagacgcccatctttgaccctacagttcattggctgttcaccacatgtggggccagcggaccccatggtcccacccaggcgcagtgcaacaacgcctaccagaactccaacctgagcgtggaggtgggaagcgagggccccctgaagggcatccagatctggaaggtgccagccaccgacacctacagcatctcgggctacggagctgctggcgggaaaggcgggaagaacaccatgatgcggtcccacggcgtgtctgtgctgggcatcttcaacctggagaaagacgacacgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggaggaggaggagggggtggagccacctacgtatttaagatgaaggatggagtgccggtgcctctgatcattgcagccggaggtggcggcagggcctatggggccaagacagacgcgttccacccagagagactggagaataactcctcggttctagggctgaacggcaattccggagccgcaggtggtggaggtggctggaatgataacacttccttgctctgggccggaaaatctttgctggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccatggggaagtgaatattaagcattatctaaactgcagtcactgtgaggtagacgaatgtcacatggacactgaaagccacaaggtcatctgcttctgtgaccacgggacagtgctggcagaggatggcgtctcctgcattgtgtcgcccaccccggagccgcacctgccactctcactgatcctctccgtggtgacctctgccctcgtggctgccctggtcctggctttctccggcatcatgattgtgtaccgccggaagcaccaggagctgcaagccatgcagatggagctgcagagccctgagtacaagctgagtaagctccgcacctcaaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggagtgcccaacgacccaagccccctgcaagtggctgtgaagacgctgcctgaggtgtgctcagaacaggacgaactggatttcctcatggaagccctgatcatcagcaaattcaaccaccagaacatcgttcgctgcatcggggtgagcctgcaggccctgccccggttcatcctactggagctcatggcaggaggagacctcaagtccttcctccgagagacccgccctcgcccgagccagccctcctccctggccatgctggaccttctgcatgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcaagctactacagaaagggaggctgtgcgatgctgccagttaagtggatgcccccagaggccttcatggaaggaatattcacctctaaaacagacacatggtcctttggagtgctgctgtgggaaatcttttctctcggatatatgccatatcccagcaaaagcaaccaggaagttctggagttcgtcaccagtggaggccggatggacccacccaagaactgccctgggcctgtataccggataatgactcagtgctggcaacatcagcctgaagacaggcccaactttgccatcattttggagaggattgaatactgcacccaggacccagatgtaatcaacactgctttgccgatagaatacggtccacttgtagaagaggaagagaaagtacccgtgaggcccaaggaccctgagggggttcctcctctcctggtctctcaacaggcgaaacggggggaggagcgcagcccagctgccccaccacctgtgcctaccacctcctctggcaaggctgcaaagaaacccacagctgcagaggtctctgttcgagtccctagagggccggccgtggaagggggacacgtgaatatggcattctctcagtccaaccccccttcggagttgcacaaggtccagggatccagaaacaagcccaccagcttgtggaacccaacgtacggctcctggtttacagagaaacccaccaaaaagaataatcctctagcaaagaaggagccacatgagagaggtaacctggggctggagggaagctgtactgtcccacctaacgttgcaactgggagacttccgggggcctcactgctcctagagccctcttcgctgactgccaacatgaaggaggtacctctgttcaggctacgtcacttcccttgtgggaatgtcaattacggctaccagcaacagggcttgcccttagaagccgctactgcccccggagctggtcattacgaggacaccattctgaaaagcaagaatagcatgaaccagcctgggccctgagctcggtcgcacactcacttctcttccttaggatccctaagaccgtggaggagagagaggcaatggctccttcacaaaccagagaccaaatgtcactttttgttttgtgccaacctattttgaagtgccaccaaaaaagctgtattttcaaaacgctttagaaaggttttgagcatgggttcatcctattctttcgaaagaagaaaatgtcattaaaatgagtgataaatgcaaggcccagatgtggttgcataaggtttttatgcatgtttgttgtatacttccttatgcttcttttaaattgtgtgtgctctgcttcaatgtagtcagaattagctgcttctatgtttcatagtcggggtcatagatgtttccttaccttgttgatgtggacatgagccatttgaggggagagggaacagaaataaaggagttatttgtaatgactcagcatggggaaagacattctttacttgaaaaatcatagacaactaaaatgtcactttaggtgacggttagatgcttttaattgtgctgattcatcaccaattgtaacaaatgttgtgagtagttccagtagtatagcagaaatgtgtatatactcatctaaatgaaatgcatacattccaagtgctttgagtaggataaagcaccaatacagatccagagattcaaacatcctattaatatacctcatgccttaagaaaatcctcttactaaaagtgaaaaactgagtgtgaggtttcctactggatttaaaactcaagatttaaactacttattcaaaaaatttccactaattaaactttcatcatttaaaagcaaaaattttaagcagcagtgatacatagaagcatagaaacatccatttgtgggcataagtgtcaatacattttaggccaaaaccttctgtttgtttcagtcatggactgactaccaaactcaagtttatacctgtagagatggacagggccccaggaggaacaggccgctggtggtcctaaggagaggcactgatccagtctctttccttccatggctaacctgcgttttctctccatcatgtgatccaagtacactgggaacacaaatgtgctgaaatacacatagcataatttgtatagctgtcaaagacaaattaaaacaaagaattttttccctaggaggcaatgaggaaaggaattgtggaacccctgaagagttgaggttgacattgacatgttagagtgggagttctgaacctggttttcacagatgaattttacttccatgaaaacgcattgggaatctgttcaattttacacacatacttatgtgtgtaatgcttagaatgctttatcagtaagtcctcatttcatcctctttctgcttctcctctatcctcatgcccgttttctttcctattggtagctatgccaaagcattcaagagggaagcattctcttactacttgctagtaactaaacattaccaaacaaaaggactgtgtgtgtgtatattatgaaaggtatatatatacatgcctgtttccgaccctctgtttcacctgcatcaaaagcatcactttcagcgtgggaaag

SEQ ID NO: 4 - DNA, 7385 bp, Macaca fascicularis
ctttcccacgctgaaagtgatgcttttgatgcaggtgaaacagagggtcggaaacaggcatgtatatatatacctttcataatatacacacacacagtccttttgtttggtaatgtttagttactagcaagtagtaagagaatgcttccctcttgaatgctttggcatagctaccaataggaaagaaaacgggcatgaggatagaggagaagcagaaagaggatgaaatgaggacttactgataaagcattctaagcattacacacataagtatgtgtgtaaaattgaacagattcccaatgcgttttcatggaagtaaaattcatctgtgaaaaccaggttcagaactcccactctaacatgtcaatgtcaacctcaactcttcaggggttccacaattcctttcctcattgcctcctagggaaaaaattctttgttttaatttgtctttgacagctatacaaattatgctatgtgtatttcagcacatttgtgttcccagtgtacttggatcacatgatggagagaaaacgcaggttagccatggaaggaaagagactggatcagtgcctctccttaggaccaccagcggcctgttcctcctggggccctgtccatctctacaggtataaacttgagtttggtagtcagtccatgactgaaacaaacagaaggttttggcctaaaatgtattgacacttatgcccacaaatggatgtttctatgcttctatgtatcactgctgcttaaaatttttgcttttaaatgatgaaagtttaattagtggaaattttttgaataagtagtttaaatcttgagttttaaatccagtaggaaacctcacactcagtttttcacttttagtaagaggattttcttaaggcatgaggtatattaataggatgtttgaatctctggatctgtattggtgctttatcctactcaaagcacttggaatgtatgcatttcatttagatgagtatatacacatttctgctatactactggaactactcacaacatttgttacaattggtgatgaatcagcacaattaaaagcatctaaccgtcacctaaagtgacattttagttgtctatgatttttcaagtaaagaatgtctttccccatgctgagtcattacaaataactcctttatttctgttccctctcccctcaaatggctcatgtccacatcaacaaggtaaggaaacatctatgaccccgactatgaaacatagaagcagctaattctgactacattgaagcagagcacacacaatttaaaagaagcataaggaagtatacaacaaacatgcataaaaaccttatgcaaccacatctgggccttgcatttatcactcattttaatgacattttcttctttcgaaagaataggatgaacccatgctcaaaacctttctaaagcgttttgaaaatacagcttttttggtggcacttcaaaataggttggcacaaaacaaaaagtgacatttggtctctggtttgtgaaggagccattgcctctctctcctccacggtcttagggatcctaaggaagagaagtgagtgtgcgaccgagctcagggcccaggctggttcatgctattcttgcttttcagaatggtgtcctcgtaatgaccagctccgggggcagtagcggcttctaagggcaagccctgttgctggtagccgtaattgacattcccacaagggaagtgacgtagcctgaacagaggtacctccttcatgttggcagtcagcgaagagggctctaggagcagtgaggcccccggaagtctcccagttgcaacgttaggtgggacagtacagcttccctccagccccaggttacctctctcatgtggctccttctttgctagaggattattctttttggtgggtttctctgtaaaccaggagccgtacgttgggttccacaagctggtgggcttgtttctggatccctggaccttgtgcaactccgaaggggggttggactgagagaatgccatattcacgtgtcccccttccacggccggccctctagggactcgaacagagacctctgcagctgtgggtttctttgcagccttgccagaggaggtggtaggcacaggtggtggggcagctgggctgcgctcctccccccgtttcgcctgttgagagaccaggagaggaggaaccccctcagggtccttgggcctcacgggtactttctcttcctcttctacaagtggaccgtattctatcggcaaagcagtgttgattacatctgggtcctgggtgcagtattcaatcctctccaaaatgatggcaaagttgggcctgtcttcaggctgatgttgccagcactgagtcattatccggtatacaggcccagggcagttcttgggtgggtccatccggcctccactggtgacgaactccagaacttcctggttgcttttgctgggatatggcatatatccgagagaaaagatttcccacagcagcactccaaaggaccatgtgtctgttttagaggtgaatattccttccatgaaggcctctgggggcatccacttaactggcagcatcgcacagcctccctttctgtagtagcttgccctgtagatgtctcgggccatcccgaagtctccaatcttggccactcttccagggcctggacaggtcaagaggcagtttctggcagcaatgtctcggtggatgaagtggttttcctccaaatactgacagccacaggcaatgtcccgagccacatgcagaaggtccagcatggccagggaggagggctggctcgggcgagggcgggtctctcggaggaaggacttgaggtctcctcctgccatgagctccagtaggatgaaccggggcagggcctgcaggctcaccccgatgcagcgaacgatgttctggtggttgaatttgctgatgatcagggcttccatgaggaaatccagttcgtcctgttctgagcacacctcaggcagcgtcttcacagccacttgcagggggcttgggtcgttgggcactccggacacctggccttcatacacctccccaaaggcgccatggcccagaccccgaatgagggtgatgtttttccgcggcacctccttcaggtcactgatggaggaggtcttgccagcaaagcagtagttggggttgtagtcggtcatgatggttgaggtgcggagcttactcagcttgtactcagggctctgcagctccatctgcatggcttgcagctcctggtgcttccggcggtacacaatcatgatgccggagaaagccaggaccagggcagccacgagggcagaggtcaccacggagaggatcagtgagagtggcaggtgcggctccggggtgggcgacacaatgcaggagacgccatcctctgccagcactgtcccgtggtcacagaagcagatgaccttgtggctttcagtgtccatgtgacattcgtctacctcacagtgactgcagtttagataatgcttaatattcacttccccatggccttccatcacttttaaagctggggtgtacaggatgcccagtggactgatgaaggaaaccccatcttccccatccatttcggggtcattgtttgaggctgcattgccgcctatatatcctccgcctcctccacctgaggagcacccccctccaccccctccgaaaccccctcttgtctcccacccccacttcttcatggcctgggggcaggaatgtcctccggtggcaccctccagcaaagattttccggcccagagcaaggaagtgttatcattccagccacctccaccacctgcggctccggaattgccgttcagccctagaaccgaggagttattctccagtctctctgggtggaacgcgtctgtcttggccccataggccctgccgccacctccggctgcaatgatcagaggcaccggcactccatccttcatcttaaatacgtaggtggctccaccccctcctcctcctcctcctgcccactcatgcacgcttctgttcacacggatttcttcttctatcacattgttctctccaatgcagactttctggattaactggtttgtactggggcaggcgtcctctccctgctgcccaaccaggatgtacagcgtgtcgtctttctccaggttgaagatgcccagcacagacacgccgtgggaccgcatcatggtgttcttcccgcctttcccgccagcagctccgtagcccgagatgctgtaggtgtcggtggctggcaccttccagatctggatgcccttcagggggccctcgcttcccacctccacgctcaggttggagttctggtaggcgttgttgcactgcgcctgggtgggaccatggggtccgctggccccacatgtggtgaacagccaatgaactgtagggtcaaagatgggcgtctgtcgtggtgaattttccccgggtttcagctccttgtttgggtttctctcaaacaggtttcttgatttgggtgctgtattctgtaggatcttgtcctctccactaatggtgaggtagcagtccaggctgatggagatattgtcaaaagccacgatggctctggatccttgtccccaccatgcaaccatctgcagccagaacctgtcagacacgtcgaggagaggcaacaccgtccactgccacaggctcaagccttcataggcggcgacatgccagaccatcctgccttgctccttcccagttttgttctccactagcaccaaggacacgtttcccctcaggactccacgaatgagccaggacattcggagctcacatggagagctcttgatcggtgcgggaaacgtagcactggtcactgtagcactttctgaagtgggggcatcagtggtactgagcaatagagcatggtcttggtggttctggaaccgggcatcctttagagtcctgacctgccactgaggagtgtgaggtgagagtgtgccttgggtccagccacagaagccatcttcgaagttgcagtaaaaacccacagggagtttccggcatatctggctctcgtcttctccctgggcgcagtcctggtggaagtcacaggcctgcccaagctggaggactgtcccattccaacaagtgaaggagctctgcagggccatcttggagcctggggatgttccttcactgcagttcttcagggcaaagaagtccactgcagacaagctgcggtttccactggagatgtattccagggccactcgaaaggggttgtctgggcgcccgattcttccctggagcactgtccaaccatgcttccctggggtgggcatcaggaggatctctcttgcagcctcgttgtggggcagcagctgggcaatgtaccttccagagggctgcaggtgcctgtgcactgagacggccagcgtacagtgctcactgctgctcctcatccacggacttaggatggtgtgcttggagtcagctgaggtgttgaggaggagaaaggaccctctgggcatctccttagaacgctctgcctcaggcccatccagcaagtccatctgggaggcctcctcggagggaacacggcgccaggaccagctctggttcctgaggtcgtgcagtggaggggaatattccagctcacaggggaagtcaaagctgcactccagaccatatcggctgcgatgagacaggaaagggaaggagtctttcattatccaggtgagattccatgtaaaataatcaggagaaggagaaggtatgtttgttggtgactccaaggagctatgaccagtcccgaagatctggaagagaaggcggggctgggaggcgcggattgccgcggacagccttccctctctgcccacttccgacgccttcttctcgggcatcaggcggatcctcagtcgcccttcgccctggcgaatccaccaactgaacagctcgctgaggttgaactggagcagccccacagccgcctccccagggggcccgacgcaaccctccaagatcgcctcctcgcccagctccagcaccagctgcttggcacgccggagcttgcgcaccgagccgcccttcagcaccctggacagcgtccgggcctctgccggggtgggtgagccggctgtccaggagaccccgggcgccggccccagcaacctgagcagcggggcgcagtccagagcgagcgagcctcgcgcctcgggcctgccagccttcagctccgagggggatggcggcagcagcaggtcccgggcgtagacacggaagagtgagggcactacgaagtccactgccagactcttcctctgcaggcgagagtagctgagcggctcccggggctgcagcggcggccccgcagctggggatcccgtgcgctggccggtccccgtcccagagcccaaagctgcagtggaaagcagcagcggcagcagccacaggagcccgatggttcccatcccgccgaaggaggccgtttacactgctctccaggcccagcctcgcccttcgctctcccctagatgggaagaagctctgaacagtcctcggtacccagcggctccttccacctgatctccagaggactgtgcgtgagcgcaagtctcttgctttcccccaactgcacggaggcgagcaggagtctcaaggaaacacagctgtaatctcaggggcgagtccagagaccctcaagcacgctgggctcactgactgggaccttgagtctcccgctcgccacgccgagtgccgctcccccgtcagtagctcgctgcgctccgcacagaggaactactatggttgaagggaggtggcagttgggtaccgtcctctcctgccccccgcggtcggagctggggtctgtcccctctcggggcagcctccaatctctgcaactttccagactgagaacggcggctcccagctgctgcacgctgtcctggccgcctcctgcgttccttttggctcctccgagctcctctgcccggtctgggtgggatccgacgacgggggctgccttctcgcccaccctcaagctatctctccgctgcgggaaggcttcggactgctctgcccgctgaacttctagggcgtgaatccgaacccccgcgctgcgcaagtttgcagcgtccttgctgtcgcccgcgcctcgctcggttcctcagagttcgcaggcggcagcagctgagggcgttcagggcaggggctcccgaaatcttgccgacccctcctcacccattatcagcgcccgcggctttgggtggccggccagagggcggccggaaagcacatcggtgccccgcgaccctccgaacagaggcggcgggaggtaccagctgctaccgccgctgccgcccccagagctgctggatcgcatctgcctgggcgcccgccacttgcagct

SEQ ID NO: 5 - DNA, 5918 bp, Mus musculus
gtgttcacgcccagaagttcagcgggcagggtgatcgatccgaagacttcctgcagcggaggtcacttgagggggcgctagaaagcagccccctccggtggtccttgcctagacctgggaaggagcgcagaggaggtgacaggagcggaggacgtgggcaagacagtgaccgactcggagccacggttcacagcctggaaagttgcagaagattggaagctaagaggagagctctggtcgccgagggctccttgaacggtacctaattgccacctccctggtccctgagcaaaggcctctacaaatggggcgcagcacggcgagaggcgcaggatccagctgttgagcccagggtgtctcactgtctccgaactaccccctgactttgtcttccgttttgctgagaacccttctcgcctccttgtagcttgggaaaagcaagggcgctctatagtgtacacacagtccctgagatctagtggaaggagccattcaggaccaaggactatttggagccctttcctgtttgggggagagtgaagggcgaggctggaccagcaagggaagggagactagtgtaaactcgccctccagcgggatgggagctgctgggttcctgtggctgctgcctccactgcttttggcagcagcctcgtactccggagctgcaaccgatcagcgcgcgggttccccagcctcagggcctcctctgcagccccgggagccgctcagttattcgcgcctgcagaggaagagtctggcagtggacttcgttgtaccctcgctcttccgcgtctatgcccgagacctgctgctaccgcagccacggtccccctcggagcccgaggctggcgggctggaggcgcggggatcactggccctggattgtgagcctctgctcaggctgctggggccactgcctggaatctcctgggcagatggagccagttctcctagtcccgaggcgggtccgacgctgtccagggtgctgaagggaggctcggtgcgcaagctcaggcgtgccaaacagctggtgctggagctgggcgaggagacgattcttgaaggctgtattggtcccccagaggaggtagcggctgtggggatactccagttcaacctcagcgagctgttcagctggtggattctccacggcgaagggaggctgaggatccgcctgatgcctgagaagaaggcatcggaagtgggcagggagggaaggctatccagtgcgatccgagcctcccagccccgccttctcttccagatcttcgggacgggacacagctccatggagtcaccctcagaaacgccttctcctcctggtaccttcatgtggaatctgacctggacgatgaaagactccttccctttcctttcccaccgcagtcgatatggtctggagtgcagctttgacttcccctgtgagctggaatattctcctcccctgcacaaccacgggaatcagagctggtcctggcgccatgtgccctccgaggaggcctcgaggatgaacttgctggatgggccagaggcagagcattctcaagagatgcccagaggctccttcctcctcctgaacacctctgcagattccaagcataccattctgagcccatggatgaggagcagtagtgatcactgcacactggctgtctccgtgcacagacatctacagccttcggggagatatgttgcccagctcctaccccacaacgaagctggaagagagattcttttggtgcccaccccagggaagcatggctggacagtgctgcaggggagagtcgggcgcccagcaaacccatttcgagtggctctggaatacatctccagtggcaaccggagcttgtcggcggtggatttctttgccctgaagaactgcagtgaagggacatccccaggctccaagatggcattgcagagttccttcacttgttggaatgggaccgtcctccagctcgggcaagcctgtgatttccaccaggactgtgcccaaggagaagatgagggccagctgtgcagtaaacttcctgctggattttactgtaactttgaaaatggcttctgtggctggacccaaagtccactctcaccccatatgccccggtggcaagtgaggaccctaagagatgcccattcccagggccaccaaggccgtgccctgttgctcagcaccactgacatcctcgcttctgaaggtgcaacagtgaccagtgccaccttccctgcaccaatgaaaaattctccttgtgagctccgcatgtcctggctcatccgcggggttttgagaggaaacgtatctctggtgctggtggagaacaaaaccggaaaggagcaaagccggactgtctggcatgtcgccactgacgaaggcttaagcctgtggcagcatacagtgctgtccctcctcgatgtgactgacaggttctggctgcagatagtcacatggtggggtccaggatccagggcaaccgtgggatttgacaacatttccatcagcctcgactgctaccttaccatcagtggagaggagaaaatgtccctgaattcagtacccaaatctagaaatctgtttgagaaaaacccaaacaaggagtcaaaatcctgggcaaacatatcaggaccaactcccatcttcgaccctacagttcactggctgttcaccacgtgtggggccagtggacctcatggccccacccaggcacagtgcaacaacgcctaccagaattccaacttgagcgtggtggtgggaagtgaagggcccttgaagggagtccagatttggaaagtgccagctactgacacctacagtatctcgggctacggagcagctggcgggaaaggtgggaaaaacaccatgatgcggtcccatggcgtgtctgtcctgggcatcttcaatctggagaaaggtgacacactctacatccttgtcggtcagcaaggggaggatgcctgtcccagggcaaaccaactaatccagaaagtctgtgtgggtgagaacaatgtcatagaagaagagatccgagtgaacagaagcgtgcacgagtgggcaggaggaggaggaggtgggggtggagccacctacgtgtttaagatgaaagatggcgtgcctgtacccctgatcattgcagctggtggtggtggcagggcctatggggccaagacagaaacgttccacccagagagactggagagtaactcctcggttctagggctgaacggcaattccggagccgcaggtggtggaggcggctggaatgataacacttccttgctctgggccggaaagtctttgctggagggtgccgccggaggacattcctgcccccaggccatgaagaagtgggggtgggagacaagagggggtttcggagggggtggaggggggtgctcctcaggtggaggaggcggaggatatataggtggcaacgcagcatcaaacaatgaccccgaaatggatggggaagatggggtttccttcatcagtccattgggtatcctgtacaccccggccttaaaagtgatggagggccacggggaagtgaatatcaagcattatctaaactgcagtcactgcgaggtagacgaatgtcacatggaccccgagagccacaaagtcatctgcttctgtgatcatgggaccgtgctggctgatgatggtgtctcctgcattgtgtcacccaccccggagccccacctgccgctctcattgatcctctccgtcgtgacctctgccctggtggctgcccttgttctggcattctccggcatcatgattgtgtaccgtcggaagcaccaggagttgcaggctatgcagatggaactgcagagccccgagtataagctgagcaagctacggacctcgaccatcatgaccgactacaaccccaactactgcttcgctggcaagacttcctccatcagtgacctgaaagaagtgccacggaaaaacatcacactcatccggggcctaggccatggcgcatttggggaggtgtatgaaggccaggtgtctggaatgcccaatgacccaagccctctacaagtggctgtaaagacgctgccagaagtgtgttcagaacaagatgagctggactttctcatggaagctctgatcatcagcaaattcaaccaccagaatattgttcgctgcatcggggtgagtctacaagccctgccccgcttcatcctgctggaactcatggctggcggagacctcaagtccttcctcagggagacacgccctcgcccgaaccaacccacctccctggccatgctggaccttctgcatgtggctcgggacattgcctgtggctgtcagtacctagaggagaatcactttatccaccgggatattgctgctagaaactgtctgttgacctgcccaggagctggaagaatagcaaagattggagactttgggatggcccgagatatctacagggccagctactaccgaaagggaggctgcgccatgctgccggtcaagtggatgccccctgaagccttcatggaagggatatttacctctaaaacagacacatggtcttttggagtgttgctatgggaaatattttctcttggatatatgccgtaccccagcaagagcaaccaggaagttctggagtttgtcaccagcggaggacggatggacccgcctaagaactgccccgggcctgtataccggataatgacgcagtgctggcagcatcagcctgaagacagacccaacttcgccatcattttggagaggatcgaatactgcacccaggaccccgatgtgatcaacacagctctgcccatcgaatacggtccagtagtagaagaggaggagaaagtgcccatgcgccccaaagaccccgaggggatgccacctttgctggtgtctccccagcctgcgaagcacgaggaggcgtccgcagctccccagcccgcagccctgacggcaccaggcccatcggtgaagaagcccccgggtgcgggtgcgggcgcgggcgcgggtgcgggtgccggcccggtgccccgaggtgcggccgatcggggccacgtgaacatggctttctctcagcccaaccctcccccggagctgcacaaaggcccgggatccagaaacaagccgaccagcctgtggaaccccacctacggctcgtggttcaccgagaagcctgccaaaaagacccatcctccgccaggcgccgagccgcaggcgcgggcaggagcggccgagggtggctggaccgggccgggcgcggggccccgcagagccgaggcagcgctgctgctagagccatcggcgctgagcgccaccatgaaggaggtgccgctgttcaggctgcgccacttcccctgcggcaatgtcaactatggttaccagcaacagggtctccccttggaagccacagccgcgccaggggacaccatgctgaaaagcaagaataaggtcacccagccggggccctgagccctgtactccactagcttctcctcctggcggagccggagcccacccagagggagatggacaggatggctccaccacaaacccaagaccaaaactttcatttttgtgccaacttgttttgaagtgccacattttaaaaaaaggaaacttgtgtttttaagatgtgttagaaggttttttgagcatgggttcatctatcctctcaaaagaagaaaatgccattctttaaaaaagaaaaaaaagcaatcagtgcaaggcccagattggttgcgccaagttttcgtgcatggtctgctgtacagtcccctaaggcttctttccgatttttgtgtgcgctctgcttccgcgtagtcagaaatagctgcttccatgtctcatagggggagtcctaggtgtttcctttgccttatgaatatgaaccactcgaggggcgggcgagggaacagaaataaag

SEQ ID NO: 6 - DNA, 5918 bp, Mus musculus
ctttatttctgttccctcgcccgcccctcgagtggttcatattcataaggcaaaggaaacacctaggactccccctatgagacatggaagcagctatttctgactacgcggaagcagagcgcacacaaaaatcggaaagaagccttaggggactgtacagcagaccatgcacgaaaacttggcgcaaccaatctgggccttgcactgattgcttttttttcttttttaaagaatggcattttcttcttttgagaggatagatgaacccatgctcaaaaaaccttctaacacatcttaaaaacacaagtttcctttttttaaaatgtggcacttcaaaacaagttggcacaaaaatgaaagttttggtcttgggtttgtggtggagccatcctgtccatctccctctgggtgggctccggctccgccaggaggagaagctagtggagtacagggctcagggccccggctgggtgaccttattcttgcttttcagcatggtgtcccctggcgcggctgtggcttccaaggggagaccctgttgctggtaaccatagttgacattgccgcaggggaagtggcgcagcctgaacagcggcacctccttcatggtggcgctcagcgccgatggctctagcagcagcgctgcctcggctctgcggggccccgcgcccggcccggtccagccaccctcggccgctcctgcccgcgcctgcggctcggcgcctggcggaggatgggtctttttggcaggcttctcggtgaaccacgagccgtaggtggggttccacaggctggtcggcttgtttctggatcccgggcctttgtgcagctccgggggagggttgggctgagagaaagccatgttcacgtggccccgatcggccgcacctcggggcaccgggccggcacccgcacccgcgcccgcgcccgcacccgcacccgggggcttcttcaccgatgggcctggtgccgtcagggctgcgggctggggagctgcggacgcctcctcgtgcttcgcaggctggggagacaccagcaaaggtggcatcccctcggggtctttggggcgcatgggcactttctcctcctcttctactactggaccgtattcgatgggcagagctgtgttgatcacatcggggtcctgggtgcagtattcgatcctctccaaaatgatggcgaagttgggtctgtcttcaggctgatgctgccagcactgcgtcattatccggtatacaggcccggggcagttcttaggcgggtccatccgtcctccgctggtgacaaactccagaacttcctggttgctcttgctggggtacggcatatatccaagagaaaatatttcccatagcaacactccaaaagaccatgtgtctgttttagaggtaaatatcccttccatgaaggcttcagggggcatccacttgaccggcagcatggcgcagcctccctttcggtagtagctggccctgtagatatctcgggccatcccaaagtctccaatctttgctattcttccagctcctgggcaggtcaacagacagtttctagcagcaatatcccggtggataaagtgattctcctctaggtactgacagccacaggcaatgtcccgagccacatgcagaaggtccagcatggccagggaggtgggttggttcgggcgagggcgtgtctccctgaggaaggacttgaggtctccgccagccatgagttccagcaggatgaagcggggcagggcttgtagactcaccccgatgcagcgaacaatattctggtggttgaatttgctgatgatcagagcttccatgagaaagtccagctcatcttgttctgaacacacttctggcagcgtctttacagccacttgtagagggcttgggtcattgggcattccagacacctggccttcatacacctccccaaatgcgccatggcctaggccccggatgagtgtgatgtttttccgtggcacttctttcaggtcactgatggaggaagtcttgccagcgaagcagtagttggggttgtagtcggtcatgatggtcgaggtccgtagcttgctcagcttatactcggggctctgcagttccatctgcatagcctgcaactcctggtgcttccgacggtacacaatcatgatgccggagaatgccagaacaagggcagccaccagggcagaggtcacgacggagaggatcaatgagagcggcaggtggggctccggggtgggtgacacaatgcaggagacaccatcatcagccagcacggtcccatgatcacagaagcagatgactttgtggctctcggggtccatgtgacattcgtctacctcgcagtgactgcagtttagataatgcttgatattcacttccccgtggccctccatcacttttaaggccggggtgtacaggatacccaatggactgatgaaggaaaccccatcttccccatccatttcggggtcattgtttgatgctgcgttgccacctatatatcctccgcctcctccacctgaggagcacccccctccaccccctccgaaaccccctcttgtctcccacccccacttcttcatggcctgggggcaggaatgtcctccggcggcaccctccagcaaagactttccggcccagagcaaggaagtgttatcattccagccgcctccaccacctgcggctccggaattgccgttcagccctagaaccgaggagttactctccagtctctctgggtggaacgtttctgtcttggccccataggccctgccaccaccaccagctgcaatgatcaggggtacaggcacgccatctttcatcttaaacacgtaggtggctccacccccacctcctcctcctcctgcccactcgtgcacgcttctgttcactcggatctcttcttctatgacattgttctcacccacacagactttctggattagttggtttgccctgggacaggcatcctccccttgctgaccgacaaggatgtagagtgtgtcacctttctccagattgaagatgcccaggacagacacgccatgggaccgcatcatggtgtttttcccacctttcccgccagctgctccgtagcccgagatactgtaggtgtcagtagctggcactttccaaatctggactcccttcaagggcccttcacttcccaccaccacgctcaagttggaattctggtaggcgttgttgcactgtgcctgggtggggccatgaggtccactggccccacacgtggtgaacagccagtgaactgtagggtcgaagatgggagttggtcctgatatgtttgcccaggattttgactccttgtttgggtttttctcaaacagatttctagatttgggtactgaattcagggacattttctcctctccactgatggtaaggtagcagtcgaggctgatggaaatgttgtcaaatcccacggttgccctggatcctggaccccaccatgtgactatctgcagccagaacctgtcagtcacatcgaggagggacagcactgtatgctgccacaggcttaagccttcgtcagtggcgacatgccagacagtccggctttgctcctttccggttttgttctccaccagcaccagagatacgtttcctctcaaaaccccgcggatgagccaggacatgcggagctcacaaggagaatttttcattggtgcagggaaggtggcactggtcactgttgcaccttcagaagcgaggatgtcagtggtgctgagcaacagggcacggccttggtggccctgggaatgggcatctcttagggtcctcacttgccaccggggcatatggggtgagagtggactttgggtccagccacagaagccattttcaaagttacagtaaaatccagcaggaagtttactgcacagctggccctcatcttctccttgggcacagtcctggtggaaatcacaggcttgcccgagctggaggacggtcccattccaacaagtgaaggaactctgcaatgccatcttggagcctggggatgtcccttcactgcagttcttcagggcaaagaaatccaccgccgacaagctccggttgccactggagatgtattccagagccactcgaaatgggtttgctgggcgcccgactctcccctgcagcactgtccagccatgcttccctggggtgggcaccaaaagaatctctcttccagcttcgttgtggggtaggagctgggcaacatatctccccgaaggctgtagatgtctgtgcacggagacagccagtgtgcagtgatcactactgctcctcatccatgggctcagaatggtatgcttggaatctgcagaggtgttcaggaggaggaaggagcctctgggcatctcttgagaatgctctgcctctggcccatccagcaagttcatcctcgaggcctcctcggagggcacatggcgccaggaccagctctgattcccgtggttgtgcaggggaggagaatattccagctcacaggggaagtcaaagctgcactccagaccatatcgactgcggtgggaaaggaaagggaaggagtctttcatcgtccaggtcagattccacatgaaggtaccaggaggagaaggcgtttctgagggtgactccatggagctgtgtcccgtcccgaagatctggaagagaaggcggggctgggaggctcggatcgcactggatagccttccctccctgcccacttccgatgccttcttctcaggcatcaggcggatcctcagcctcccttcgccgtggagaatccaccagctgaacagctcgctgaggttgaactggagtatccccacagccgctacctcctctgggggaccaatacagccttcaagaatcgtctcctcgcccagctccagcaccagctgtttggcacgcctgagcttgcgcaccgagcctcccttcagcaccctggacagcgtcggacccgcctcgggactaggagaactggctccatctgcccaggagattccaggcagtggccccagcagcctgagcagaggctcacaatccagggccagtgatccccgcgcctccagcccgccagcctcgggctccgagggggaccgtggctgcggtagcagcaggtctcgggcatagacgcggaagagcgagggtacaacgaagtccactgccagactcttcctctgcaggcgcgaataactgagcggctcccggggctgcagaggaggccctgaggctggggaacccgcgcgctgatcggttgcagctccggagtacgaggctgctgccaaaagcagtggaggcagcagccacaggaacccagcagctcccatcccgctggagggcgagtttacactagtctcccttcccttgctggtccagcctcgcccttcactctcccccaaacaggaaagggctccaaatagtccttggtcctgaatggctccttccactagatctcagggactgtgtgtacactatagagcgcccttgcttttcccaagctacaaggaggcgagaagggttctcagcaaaacggaagacaaagtcagggggtagttcggagacagtgagacaccctgggctcaacagctggatcctgcgcctctcgccgtgctgcgccccatttgtagaggcctttgctcagggaccagggaggtggcaattaggtaccgttcaaggagccctcggcgaccagagctctcctcttagcttccaatcttctgcaactttccaggctgtgaaccgtggctccgagtcggtcactgtcttgcccacgtcctccgctcctgtcacctcctctgcgctccttcccaggtctaggcaaggaccaccggagggggctgctttctagcgccccctcaagtgacctccgctgcaggaagtcttcggatcgatcaccctgcccgctgaacttctgggcgtgaacac

SEQ ID NO: 7 - DNA, 6372 bp, Rattus norvegicus
gcagctctgggggcggcagcggcggtggcaactggctcctcccgcttcttctgtccaggggttctcggggcagtaagacgctttccgttcgccccctggccagtcaacaaaaccgcgggcgctgctggtaggtaaggagggagctggctacgttgcgggctcctctgccccgaatgttttcactcggcggcacggccggggcgctctggtgcttgggagtgaactccgaggaacgaagacgtcggtgacagcaaggacgctgcaaacttgtgcgcgtggaggcagcgggacctcctctccgcaacgcagagctcgcgttcacgcccagaagttcagcgggcagggtgatcgatccgaagacttcctgcagcggaggtcacttgagggggcgctagaaagcagccccctccggtggtccttgcctagacctgggaaggagcgcagaggaggtgacaggaacggaggacgtgggcaagacagtgaccgactcagaaccgcggctcgcagcctggaaagttgcagaagattggaggcgaagaggagagctctggtcaccgagggctccttgaacggtacctaattgccacctccctcaaccatagtgatttctctggtccctgagcaaatggggcgcagcatggcgagaggcgcaggatccagctgttgaacccaaggtgtctcactgtctccgaactcccccctgactttctcatccgttttgctgagaaccctttttgcctccttgtagactggggaaagcagggcgctctatagtgtacacagtccttgagaccaagtggaaggagccgttcaggaccaaggactatttggagcccctttctgtttaggggagagtgaaaggtgaggctggaccaggcagggaagggagtctagtgtaaactcgccctccggagggatgggagcccttgggttcctgtggctgctgccgccgctgcttttgacagcagcctcgtactccggagctgcaaccgatcagcgcgcgggttctccagcctcagggccgcctctgcagccccgggagccgctcagctattcgcgcctgcagaggaagagtctggcggtggacttcgtggtgccctcactcttccgcgtctatgcccgagacctgctgctgccgcagccaccgtccccctcggagcccggggctggcgggctggaggcgcggggatcactggctctggactgcgaccctctgctcaggttgctggggccatcgcctggaatctcctgggcagagggagccagttctcctagtccggaggcggctccgacgctgtccagggtgctgaagggcggctcggtgcgcaagctcaggcgtgccaaacagctggtgctggagctgggcgaggagacgatccttgaaggctgcattggtcccccagaggaggcggcggctgtggggatactccagttcaaccttagcgagctgttcagctggtggattctacacggcgaagggcggctgaggatccgcctgatgcctgagaagaaggcatcggaagtgggcagggagggaaggctatccactgcgatccgagcctcccagccccgccttctcttccagatcttcgggaccggtcacagctccttggaatcaccctcagaaatgccttctcctcctggtaactttctatggaatctcacctggacaatgaaagactccttccctttcctttcccaccgcagtcgatatggtctggagtgcagcttcgattttccctgtgagctagaatactcccctcccctccacacccatgggaaccagagctggtcctggcgccgtgtgccctctgaggaggcctcgaggatgaacttgttggatgggccagaggcagagcatttgaaagagatgcccagaggctccttcctcctcctgaacacctctgcagattccaagcataccatcctgagcccctggatgaggagcagtagtgagcactgcacgctggctgtctcagtgcacagacatctacagccttctgggagatatgttgcccagctcctaccccacaatgaagctggaagagagattcttctggtgcctaccccggggaaacatggctggacagtgctgcatgggagagtcgggcgcccagagaacccattccgagtggccctggaatacatctcgagtggaaaccggagcttgtctgcggtggatttctttgccctgaagaactgcagtgaaggaacatccccaggctccaagatggccttgcagagttccttcacttgttggaacgggacagtcctccagctcgggcaagcctgtgacttccaccagaactgtgcccaaggagaagatgagggtcagctatgcagtaaacttcctgctgggttttactgtaacttcgaggatggcttctgtggctggacccaaagtccactctcaccccgtgtgccccgatggcaagtgaagaccctgaaagatacccattcccagggccaccaaggccatgccctgttgctcagcaccactgacgaccccacttcagaaagtgcaacagtgaccagtgctacattccccgcaccaatgaagagctctccttgtgagctccggatgtcctggctcatccgtggggttctgaaaggaaatgtatctttggtgctggtggagaacaaaactggaaaggagcaaagccggaccgtctggcatgttgccaccaatgaaggcctaagcctgtggcagtggacagtgttgtcgctcctcgatgtgactgacaggttctggctgcaaatagtcacatggtggggcccaggatccagggcaaccgtggcatttgacaacatttccatcagcctcgactgctacctcaccatcagtggggaggagaaaatgtccctgaatgcagtacccaaatccagaaatctgtttgagaaaaacccaaacaaggagccaaaaccctgggcaaacatatcaggaccaactcccatcttcgaccctacagttcactggctgttcaccacatgtggggccagtggccctcatggccccacacaggcacagtgcaacaacgcctaccagaactccaacctgagcgtggtggtgggaagtgaagggcccttgaggggcatccagatttggaaagtgccagctactgacacctacagtatctcaggctatggagcagctggcggaaaaggtgggaagaacaccatgatgcggtcccatggcgtgtctgtcctgggcatcttcaatctggagaaagatgacacgctctacatccttgtcggtcaacaaggggaagatgcctgtcccagggcaaaccaactaatccagaaagtatgtgtcggcgagaacaatgtgatagaagaagagatccgagtgaacagaagcgttcacgagtgggcaggaggaggaggaggtgggggtggagccacctacgtgtttaagatgaaagacggcgtgcccgtgcccctgattattgcggccggcggtggtggcagggcctatggggccaagacagaaacgttccacccagagagactggagaataactcctcagttctagggctgaacggcaattccggagccgcaggtggtggaggcggctggaatgataacacttccttgctctgggccggaaagtctttgttggagggcgccgccggaggacattcctgcccccaggccatgaagaagtgggggtgggagacaagagggggtttcggagggggtggaggggggtgctcctcaggtggaggaggcggaggatatataggtggcaacgcagcatcaaacaatgaccccgaaatggatggggaagatggggtttccttcatcagtccattgggtatcctgtataccccggccttaaaagtgatggagggccatggggaagtgaatatcaagcactatctaaactgcagccactgtgaggtagacgaatgtcacatggaccccgagagccacaaagtcatctgcttctgtgaccatgggaccgtgctggctgacgatggtgtctcctgcattgtgtcgcccaccccggaaccccacctgccgctctcattgatcctctccgtcgtgacctctgccctggtggccgctctcgttctggcattctccggcatcatgattgtgtaccgccggaagcaccaggagttgcaagctatgcagatggagctgcagagccctgagtataagctgagcaagctacggacctcgaccatcatgacagactacaaccccaactactgctttgctggcaagacttcatccatcagtgacctgaaggaggtgcctcggaaaaacatcaccctcatccggggcctaggccatggcgcatttggggaggtgtatgaaggccaggtatctggaatgcccaatgacccaagccctctacaagtggctgtaaagacgctgccggaagtgtgttcagagcaagatgagctggacttcctcatggaagctctgatcatcagtaaattcaaccaccagaacattgtccgctgcatcggggtgagtctgcaagccctgccccgcttcatcctgctggagctcatggctggtggagacctcaagtccttcctcagggagacacgtcctcgcccgaaccagcccacctccctggccatgttggatcttctgcacgtagctcgggacattgcctgtggctgtcagtacctggaggaaaatcactttatccaccgcgatattgctgctagaaactgtctgctgacctgtccgggagctgggaggatagcgaagatcggagacttcgggatggcccgagatatctacagggccagctactacagaaagggaggctgtgccatgctgccagtgaagtggatgcccccggaggccttcatggaaggaatatttacttctaaaacagatacatggtcttttggagtgttgctgtgggaaatattttctcttggatatatgccataccccagcaagagcaaccaggaagttctggagtttgtcaccagtggaggacggatggacccacctaagaactgccccggacctgtataccggataatgactcagtgctggcagcatcagcctgaggacagacccaactttgccatcattctggagaggatcgaatactgcacccaggaccccgatgtcatcaatacagctctgcccatcgaatatggccccgtagtagaggaggaggagaaagtgcccatgcgccccaaagaccctgaggggatgcctcctctgctggtgtctccccagtctgcgaagcacgaggaggcgtcctcagccccccaaccctcagccctggcagcgccaggcccgctggtgaagaagccctcgggtgcaggcgcgggagcgggtgccggtccggtgccccgaggtgcagccgatcggggccacgtgaacatggcgttctctcagcccaaccctcccccagagctgcacaaaggcccgggctccagaaacaagccgaccagcctgtggaaccccacctacggctcgtggttcaccgagaagccagccaaaaagacccatcccccaccgggcgcggagccgcaggcccgggcaggagcggcggagggtggctggaccgggccgggtgcggggccccgcagagccgaggctgcgctgctgctcgagccgtcggcgctcagcgccaccatgaaggaggtgcccctgttcaggctgcgccacttcccctgcggcaacgtcaactatggttaccagcaacagggtctccccttggaagccactgctgccccgggggacaccgtgctgaaaagcaagactaaggtcacccagccagggccctgagcccggcgtcccactagcttttctccctggctggagctgaagcccaccgggagggagacggacagaatgactccaccacaattccgagaccaaatgtcactcactttcatttttgtgccaacttgttttgaagtgccacatttaaaagagaaaaagaagaaaaaaaaaagaggaaaacttgttttcaagatgtgttagaaaggtttttgagcatgggttcatccatcctcaaagaagaaaatgccgttcttaagaaagcgatcagtacaaggcccagattggttgcgtagttttggtgcatgatctgctgtacagtcccctcaggcttctttccgatttgtgtgtgctctctgcttccgtgtagtcagaaatagctgcctccatgtctcatagggggagtcgtaggtgtttccttgccttgtggatatgaaccattcgaggggcgagggaacagaaataaaggaattaatttcaatgactcagcatgaggagagacgttatttacgtggaaaagaaatatcatacactgtcatcgctgtgggtggtggttagaggcttttaattgttctaactcattgcctattgtaaaaaaaaaaaaaaaa

SEQ ID NO: 8 - DNA, 6372 bp, Rattus norvegicus
ttttttttttttttttacaataggcaatgagttagaacaattaaaagcctctaaccaccacccacagcgatgacagtgtatgatatttcttttccacgtaaataacgtctctcctcatgctgagtcattgaaattaattcctttatttctgttccctcgcccctcgaatggttcatatccacaaggcaaggaaacacctacgactccccctatgagacatggaggcagctatttctgactacacggaagcagagagcacacacaaatcggaaagaagcctgaggggactgtacagcagatcatgcaccaaaactacgcaaccaatctgggccttgtactgatcgctttcttaagaacggcattttcttctttgaggatggatgaacccatgctcaaaaacctttctaacacatcttgaaaacaagttttcctcttttttttttcttctttttctcttttaaatgtggcacttcaaaacaagttggcacaaaaatgaaagtgagtgacatttggtctcggaattgtggtggagtcattctgtccgtctccctcccggtgggcttcagctccagccagggagaaaagctagtgggacgccgggctcagggccctggctgggtgaccttagtcttgcttttcagcacggtgtcccccggggcagcagtggcttccaaggggagaccctgttgctggtaaccatagttgacgttgccgcaggggaagtggcgcagcctgaacaggggcacctccttcatggtggcgctgagcgccgacggctcgagcagcagcgcagcctcggctctgcggggccccgcacccggcccggtccagccaccctccgccgctcctgcccgggcctgcggctccgcgcccggtgggggatgggtctttttggctggcttctcggtgaaccacgagccgtaggtggggttccacaggctggtcggcttgtttctggagcccgggcctttgtgcagctctgggggagggttgggctgagagaacgccatgttcacgtggccccgatcggctgcacctcggggcaccggaccggcacccgctcccgcgcctgcacccgagggcttcttcaccagcgggcctggcgctgccagggctgagggttggggggctgaggacgcctcctcgtgcttcgcagactggggagacaccagcagaggaggcatcccctcagggtctttggggcgcatgggcactttctcctcctcctctactacggggccatattcgatgggcagagctgtattgatgacatcggggtcctgggtgcagtattcgatcctctccagaatgatggcaaagttgggtctgtcctcaggctgatgctgccagcactgagtcattatccggtatacaggtccggggcagttcttaggtgggtccatccgtcctccactggtgacaaactccagaacttcctggttgctcttgctggggtatggcatatatccaagagaaaatatttcccacagcaacactccaaaagaccatgtatctgttttagaagtaaatattccttccatgaaggcctccgggggcatccacttcactggcagcatggcacagcctccctttctgtagtagctggccctgtagatatctcgggccatcccgaagtctccgatcttcgctatcctcccagctcccggacaggtcagcagacagtttctagcagcaatatcgcggtggataaagtgattttcctccaggtactgacagccacaggcaatgtcccgagctacgtgcagaagatccaacatggccagggaggtgggctggttcgggcgaggacgtgtctccctgaggaaggacttgaggtctccaccagccatgagctccagcaggatgaagcggggcagggcttgcagactcaccccgatgcagcggacaatgttctggtggttgaatttactgatgatcagagcttccatgaggaagtccagctcatcttgctctgaacacacttccggcagcgtctttacagccacttgtagagggcttgggtcattgggcattccagatacctggccttcatacacctccccaaatgcgccatggcctaggccccggatgagggtgatgtttttccgaggcacctccttcaggtcactgatggatgaagtcttgccagcaaagcagtagttggggttgtagtctgtcatgatggtcgaggtccgtagcttgctcagcttatactcagggctctgcagctccatctgcatagcttgcaactcctggtgcttccggcggtacacaatcatgatgccggagaatgccagaacgagagcggccaccagggcagaggtcacgacggagaggatcaatgagagcggcaggtggggttccggggtgggcgacacaatgcaggagacaccatcgtcagccagcacggtcccatggtcacagaagcagatgactttgtggctctcggggtccatgtgacattcgtctacctcacagtggctgcagtttagatagtgcttgatattcacttccccatggccctccatcacttttaaggccggggtatacaggatacccaatggactgatgaaggaaaccccatcttccccatccatttcggggtcattgtttgatgctgcgttgccacctatatatcctccgcctcctccacctgaggagcacccccctccaccccctccgaaaccccctcttgtctcccacccccacttcttcatggcctgggggcaggaatgtcctccggcggcgccctccaacaaagactttccggcccagagcaaggaagtgttatcattccagccgcctccaccacctgcggctccggaattgccgttcagccctagaactgaggagttattctccagtctctctgggtggaacgtttctgtcttggccccataggccctgccaccaccgccggccgcaataatcaggggcacgggcacgccgtctttcatcttaaacacgtaggtggctccacccccacctcctcctcctcctgcccactcgtgaacgcttctgttcactcggatctcttcttctatcacattgttctcgccgacacatactttctggattagttggtttgccctgggacaggcatcttccccttgttgaccgacaaggatgtagagcgtgtcatctttctccagattgaagatgcccaggacagacacgccatgggaccgcatcatggtgttcttcccaccttttccgccagctgctccatagcctgagatactgtaggtgtcagtagctggcactttccaaatctggatgcccctcaagggcccttcacttcccaccaccacgctcaggttggagttctggtaggcgttgttgcactgtgcctgtgtggggccatgagggccactggccccacatgtggtgaacagccagtgaactgtagggtcgaagatgggagttggtcctgatatgtttgcccagggttttggctccttgtttgggtttttctcaaacagatttctggatttgggtactgcattcagggacattttctcctccccactgatggtgaggtagcagtcgaggctgatggaaatgttgtcaaatgccacggttgccctggatcctgggccccaccatgtgactatttgcagccagaacctgtcagtcacatcgaggagcgacaacactgtccactgccacaggcttaggccttcattggtggcaacatgccagacggtccggctttgctcctttccagttttgttctccaccagcaccaaagatacatttcctttcagaaccccacggatgagccaggacatccggagctcacaaggagagctcttcattggtgcggggaatgtagcactggtcactgttgcactttctgaagtggggtcgtcagtggtgctgagcaacagggcatggccttggtggccctgggaatgggtatctttcagggtcttcacttgccatcggggcacacggggtgagagtggactttgggtccagccacagaagccatcctcgaagttacagtaaaacccagcaggaagtttactgcatagctgaccctcatcttctccttgggcacagttctggtggaagtcacaggcttgcccgagctggaggactgtcccgttccaacaagtgaaggaactctgcaaggccatcttggagcctggggatgttccttcactgcagttcttcagggcaaagaaatccaccgcagacaagctccggtttccactcgagatgtattccagggccactcggaatgggttctctgggcgcccgactctcccatgcagcactgtccagccatgtttccccggggtaggcaccagaagaatctctcttccagcttcattgtggggtaggagctgggcaacatatctcccagaaggctgtagatgtctgtgcactgagacagccagcgtgcagtgctcactactgctcctcatccaggggctcaggatggtatgcttggaatctgcagaggtgttcaggaggaggaaggagcctctgggcatctctttcaaatgctctgcctctggcccatccaacaagttcatcctcgaggcctcctcagagggcacacggcgccaggaccagctctggttcccatgggtgtggaggggaggggagtattctagctcacagggaaaatcgaagctgcactccagaccatatcgactgcggtgggaaaggaaagggaaggagtctttcattgtccaggtgagattccatagaaagttaccaggaggagaaggcatttctgagggtgattccaaggagctgtgaccggtcccgaagatctggaagagaaggcggggctgggaggctcggatcgcagtggatagccttccctccctgcccacttccgatgccttcttctcaggcatcaggcggatcctcagccgcccttcgccgtgtagaatccaccagctgaacagctcgctaaggttgaactggagtatccccacagccgccgcctcctctgggggaccaatgcagccttcaaggatcgtctcctcgcccagctccagcaccagctgtttggcacgcctgagcttgcgcaccgagccgcccttcagcaccctggacagcgtcggagccgcctccggactaggagaactggctccctctgcccaggagattccaggcgatggccccagcaacctgagcagagggtcgcagtccagagccagtgatccccgcgcctccagcccgccagccccgggctccgagggggacggtggctgcggcagcagcaggtctcgggcatagacgcggaagagtgagggcaccacgaagtccaccgccagactcttcctctgcaggcgcgaatagctgagcggctcccggggctgcagaggcggccctgaggctggagaacccgcgcgctgatcggttgcagctccggagtacgaggctgctgtcaaaagcagcggcggcagcagccacaggaacccaagggctcccatccctccggagggcgagtttacactagactcccttccctgcctggtccagcctcacctttcactctcccctaaacagaaaggggctccaaatagtccttggtcctgaacggctccttccacttggtctcaaggactgtgtacactatagagcgccctgctttccccagtctacaaggaggcaaaaagggttctcagcaaaacggatgagaaagtcaggggggagttcggagacagtgagacaccttgggttcaacagctggatcctgcgcctctcgccatgctgcgccccatttgctcagggaccagagaaatcactatggttgagggaggtggcaattaggtaccgttcaaggagccctcggtgaccagagctctcctcttcgcctccaatcttctgcaactttccaggctgcgagccgcggttctgagtcggtcactgtcttgcccacgtcctccgttcctgtcacctcctctgcgctccttcccaggtctaggcaaggaccaccggagggggctgctttctagcgccccctcaagtgacctccgctgcaggaagtcttcggatcgatcaccctgcccgctgaacttctgggcgtgaacgcgagctctgcgttgcggagaggaggtcccgctgcctccacgcgcacaagtttgcagcgtccttgctgtcaccgacgtcttcgttcctcggagttcactcccaagcaccagagcgccccggccgtgccgccgagtgaaaacattcggggcagaggagcccgcaacgtagccagctccctccttacctaccagcagcgcccgcggttttgttgactggccagggggcgaacggaaagcgtcttactgccccgagaacccctggacagaagaagcgggaggagccagttgccaccgccgctgccgcccccagagctgc

SEQ ID NO: 9 - protein, 16 aa, Unknown
  Description of Unknown: RFGF peptide
AAVALLPAVLLALLAP

SEQ ID NO: 10 - protein, 11 aa, Unknown
  Description of Unknown: RFGF analogue peptide
AALLPVLLAAP

SEQ ID NO: 11 - protein, 13 aa, Human immunodeficiency virus
GRKKRRQRRRPPQ

SEQ ID NO: 12 - protein, 16 aa, Drosophila sp.
RQIKIWFQNRRMKWKK

SEQ ID NO: 13 - skipped

SEQ ID NO: 14 - RNA, 21 nt, Artificial Sequence
  Description of Artificial Sequence: Synthetic oligonucleotide
gcagaugcgauccagcggcua

SEQ ID NO: 15 - RNA, 21 nt, Artificial Sequence
  Description of Artificial Sequence: Synthetic oligonucleotide
cggugguagcagcugguacca

SEQ ID NO: 16 - RNA, 21 nt, Artificial Sequence
  Description of Artificial Sequence: Synthetic oligonucleotide
gcgcugaugaugggugaggaa

SEQ ID NO: 17 - RNA, 21 nt, Artificial Sequence
  Description of Artificial Sequence: Synthetic oligonucleotide
ugccugcgaacucugaggaga

SEQ ID NO: 18 - RNA, 21 nt, Artificial Sequence
  Description of Artificial Sequence: Synthetic oligonucleotide
ggacgcugcaaacuugcgcaa

SEQ ID NO: 19 - RNA, 21 nt, Artificial Sequence
  Description of Artificial Sequence: Synthetic oligonucleotide
gcugggauucacgcccagaaa

SEQ ID NO: 20 - RNA, 21 nt, Artificial Sequence
  Description of Artificial Sequence: Synthetic oligonucleotide
gcccagaaguucagcaggcaa

SEQ ID NO: 21 - RNA, 21 nt, Artificial Sequence
  Description of Artificial Sequence: Synthetic oligonucleotide
gcagacaguccgaagccuuca

SEQ ID NO: 22 - RNA, 21 nt, Artificial Sequence
  Description of Artificial Sequence: Synthetic oligonucleotide
cagcggagagauagcuugaga

SEQ ID NO: 23 - RNA, 21 nt, Artificial Sequence
  Description of Artificial Sequence: Synthetic oligonucleotide
ugagggugcgcaagacggcaa

SEQ ID NO: 24 - RNA, 21 nt, Artificial Sequence
  Description of Artificial Sequence: Synthetic oligonucleotide
gggcagaagagcuuggaggaa

SEQ ID NO: 25 - RNA, 21 nt, Artificial Sequence
  Description of Artificial Sequence: Synthetic oligonucleotide
gagccaaaaggaacgcaaaaa

SEQ ID NO: 26 - RNA, 21 nt, Artificial Sequence
  Description of Artificial Sequence: Synthetic oligonucleotide
aaaggcggccaggacagcgua

SEQ ID NO: 27 - RNA, 21 nt, Artificial Sequence
  Description of Artificial Sequence: Synthetic oligonucleotide
ccgccguucucagccuuaaaa

SEQ ID NO: 28 - RNA, 21 nt, Artificial Sequence
  Description of Artificial Sequence: Synthetic oligonucleotide
ccuuaaaaguugcagagauua

SEQ ID NO: 29 - RNA, 21 nt, Artificial Sequence
  Description of Artificial Sequence: Synthetic oligonucleotide
gacgguacccaacugccacca

SEQ ID NO: 30 - RNA, 21 nt, Artificial Sequence
  Description of Artificial Sequence: Synthetic oligonucleotide
cugccaccucccuucaaccaa

SEQ ID NO: 31 - RNA, 21 nt, Artificial Sequence
  Description of Artificial Sequence: Synthetic oligonucleotide
ucaaccauaguaguuccucua

SEQ ID NO: 32 - RNA, 21 nt, Artificial Sequence
  Description of Artificial Sequence: Synthetic oligonucleotide
guuccucuguaccgagcgcaa

SEQ ID NO: 33 - RNA, 21 nt, Artificial Sequence
  Description of Artificial Sequence: Synthetic oligonucleotide
cgagcgcagcgagcuacagaa

SEQ ID NO: 34 - RNA, 21 nt, Artificial Sequence
  Description of Artificial Sequence: Synthetic oligonucleotide
ggcucaaggucccagccagua

SEQ ID NO: 35 - RNA, 21 nt, Artificial Sequence
  Description of Artificial Sequence: Synthetic oligonucleotide
gccagugagcccagugugcua

SEQ ID NO: 36 - RNA, 21 nt, Artificial Sequence
  Description of Artificial Sequence: Synthetic oligonucleotide
agugugcuugagugucucuga

SEQ ID NO: 37 - RNA, 21 nt, Artificial Sequence
  Description of Artificial Sequence: Synthetic oligonucleotide
ggucuguuucauuuagacuca

SEQ ID NO: 38 - RNA, 21 nt, Artificial Sequence
  Description of Artificial Sequence: Synthetic oligonucleotide
cugcucgccuccgugcaguua

SEQ ID NO: 39 - RNA, 21 nt, Artificial Sequence
  Description of Artificial Sequence: Synthetic oligonucleotide
gaaagcaagagacuugcgcga

SEQ ID NO: 40 - RNA, 21 nt, Artificial Sequence
  Description of Artificial Sequence: Synthetic oligonucleotide
gcgcgcacgcacaguccucua

SEQ ID NO: 41 - RNA, 21 nt, Artificial Sequence
  Description of Artificial Sequence: Synthetic oligonucleotide
uccucuggagaucagguggaa

SEQ ID NO: 42 - RNA, 21 nt, Artificial Sequence
  Description of Artificial Sequence: Synthetic oligonucleotide
aggagccgcuggguaccaaga

SEQ ID NO: 43 - RNA, 21 nt, Artificial Sequence
  Description of Artificial Sequence: Synthetic oligonucleotide
guaccaaggacuguucagaga

SEQ ID NO: 44 - RNA, 21 nt, Artificial Sequence
  Description of Artificial Sequence: Synthetic oligonucleotide
ucagagccucuucccaucuca

SEQ ID NO: 45 - RNA, 21 nt, Artificial Sequence
  Description of Artificial Sequence: Synthetic oligonucleotide
ccggagagcaguguaaacgga

SEQ ID NO: 46 - RNA, 21 nt, Artificial Sequence
  Description of Artificial Sequence: Synthetic oligonucleotide
gggagccaucgggcuccugua

SEQ ID NO: 47 - RNA, 21 nt, Artificial Sequence
  Description of Artificial Sequence: Synthetic oligonucleotide
ugcugcuuuccacggcagcua

SEQ ID NO: 48 - RNA, 21 nt, Artificial Sequence
  Description of Artificial Sequence: Synthetic oligonucleotide
ccacucagcuacucgcgccua

SEQ ID NO: 49 - RNA, 21 nt, Artificial Sequence
  Description of Artificial Sequence: Synthetic oligonucleotide
cagaggaagagucuggcagua

SEQ ID NO: 50 - RNA, 21 nt, Artificial Sequence
  Description of Artificial Sequence: Synthetic oligonucleotide
cuggcaguugacuucguggua

SEQ ID NO: 51 - RNA, 21 nt, Artificial Sequence
  Description of Artificial Sequence: Synthetic oligonucleotide
uucguggugcccucgcucuua

SEQ ID NO: 52 - RNA, 21 nt, Artificial Sequence
  Description of Artificial Sequence: Synthetic oligonucleotide
ucgcucuuccgugucuacgca

SEQ ID NO: 53 - RNA, 21 nt, Artificial Sequence
  Description of Artificial Sequence: Synthetic oligonucleotide
gucuacgcccgggaccuacua

SEQ ID NO: 54 - RNA, 21 nt, Artificial Sequence
  Description of Artificial Sequence: Synthetic oligonucleotide
gaccuacugcugccaccauca

SEQ ID NO: 55 - RNA, 21 nt, Artificial Sequence
  Description of Artificial Sequence: Synthetic oligonucleotide
cucggagcugaaggcuggcaa

SEQ ID NO: 56 - RNA, 21 nt, Artificial Sequence
  Description of Artificial Sequence: Synthetic oligonucleotide
gcuguccagggugcugaagga

SEQ ID NO: 57 - RNA, 21 nt, Artificial Sequence
  Description of Artificial Sequence: Synthetic oligonucleotide
cgugccaagcaguuggugcua

SEQ ID NO: 58 - RNA, 21 nt, Artificial Sequence
  Description of Artificial Sequence: Synthetic oligonucleotide
uuggugcuggagcugggcgaa

SEQ ID NO: 59 - RNA, 21 nt, Artificial Sequence
  Description of Artificial Sequence: Synthetic oligonucleotide
gaggcgaucuuggaggguuga

SEQ ID NO: 60 - RNA, 21 nt, Artificial Sequence
  Description of Artificial Sequence: Synthetic oligonucleotide
cugcuccaguucaaucucaga

SEQ ID NO: 61 - RNA, 21 nt, Artificial Sequence
  Description of Artificial Sequence: Synthetic oligonucleotide
cagcgagcuguucaguuggua

SEQ ID NO: 62 - RNA, 21 nt, Artificial Sequence
  Description of Artificial Sequence: Synthetic oligonucleotide
uugguggauucgccaaggcga

SEQ ID NO: 63 - RNA, 21 nt, Artificial Sequence
  Description of Artificial Sequence: Synthetic oligonucleotide
gggcgacugaggauccgccua

SEQ ID NO: 64 - RNA, 21 nt, Artificial Sequence
  Description of Artificial Sequence: Synthetic oligonucleotide
auccgccugaugcccgagaaa

SEQ ID NO: 65 - RNA, 21 nt, Artificial Sequence
  Description of Artificial Sequence: Synthetic oligonucleotide
ccgagaagaaggcgucggaaa

SEQ ID NO: 66 - RNA, 21 nt, Artificial Sequence
  Description of Artificial Sequence: Synthetic oligonucleotide
gggcagagagggaaggcugua

SEQ ID NO: 67 - RNA, 21 nt, Artificial Sequence
  Description of Artificial Sequence: Synthetic oligonucleotide
ggcuguccgcggcaauucgca

SEQ ID NO: 68 - RNA, 21 nt, Artificial Sequence
  Description of Artificial Sequence: Synthetic oligonucleotide
ccuucucuuccagaucuucga

SEQ ID NO: 69 - RNA, 21 nt, Artificial Sequence
  Description of Artificial Sequence: Synthetic oligonucleotide
acuggucauagcuccuuggaa

SEQ ID NO: 70 - RNA, 21 nt, Artificial Sequence
  Description of Artificial Sequence: Synthetic oligonucleotide
ccuuggaaucaccaacaaaca

SEQ ID NO: 71 - RNA, 21 nt, Artificial Sequence
  Description of Artificial Sequence: Synthetic oligonucleotide
caaacaugccuucuccuucua

SEQ ID NO: 72 - RNA, 21 nt, Artificial Sequence
  Description of Artificial Sequence: Synthetic oligonucleotide
cuucuccugauuauuuuacaa

SEQ ID NO: 73 - RNA, 21 nt, Artificial Sequence
  Description of Artificial Sequence: Synthetic oligonucleotide
uuuuacauggaaucucaccua

SEQ ID NO: 74 - RNA, 21 nt, Artificial Sequence
  Description of Artificial Sequence: Synthetic oligonucleotide
ucucaccuggauaaugaaaga

SEQ ID NO: 75 - RNA, 21 nt, Artificial Sequence
  Description of Artificial Sequence: Synthetic oligonucleotide
augaaagacuccuucccuuua

SEQ ID NO: 76 - RNA, 21 nt, Artificial Sequence
  Description of Artificial Sequence: Synthetic oligonucleotide
uucccuuuccugucucaucga

SEQ ID NO: 77 - RNA, 21 nt, Artificial Sequence
  Description of Artificial Sequence: Synthetic oligonucleotide
caucgcagccgauauggucua

SEQ ID NO: 78 - RNA, 21 nt, Artificial Sequence
  Description of Artificial Sequence: Synthetic oligonucleotide
ggagugcagcuuugacuucca

SEQ ID NO: 79 - RNA, 21 nt, Artificial Sequence
  Description of Artificial Sequence: Synthetic oligonucleotide
ccacugcaugaccucaggaaa

SEQ ID NO: 80 - RNA, 21 nt, Artificial Sequence
  Description of Artificial Sequence: Synthetic oligonucleotide
caggaaccagagcugguccua

SEQ ID NO: 81 - RNA, 21 nt, Artificial Sequence
  Description of Artificial Sequence: Synthetic oligonucleotide
ucccagauggacuugcuggaa

SEQ ID NO: 82 - RNA, 21 nt, Artificial Sequence
  Description of Artificial Sequence: Synthetic oligonucleotide
agagcguucuaaggagaugca

SEQ ID NO: 83 - RNA, 21 nt, Artificial Sequence
  Description of Artificial Sequence: Synthetic oligonucleotide
augcccagaggcuccuuucua

SEQ ID NO: 84 - RNA, 21 nt, Artificial Sequence
  Description of Artificial Sequence: Synthetic oligonucleotide
uccuuucuccuucucaacaca

SEQ ID NO: 85 - RNA, 21 nt, Artificial Sequence
  Description of Artificial Sequence: Synthetic oligonucleotide
agcugacuccaagcacaccaa

SEQ ID NO: 86 - RNA, 21 nt, Artificial Sequence
  Description of Artificial Sequence: Synthetic oligonucleotide
gcacaccauccugaguccgua

SEQ ID NO: 87 - RNA, 21 nt, Artificial Sequence
  Description of Artificial Sequence: Synthetic oligonucleotide
gaguccguggaugaggagcaa

SEQ ID NO: 88 - RNA, 21 nt, Artificial Sequence
  Description of Artificial Sequence: Synthetic oligonucleotide
cagcagugagcacugcacaca

SEQ ID NO: 89 - RNA, 21 nt, Artificial Sequence
  Description of Artificial Sequence: Synthetic oligonucleotide
ugcacacuggccgucucggua

SEQ ID NO: 90 - RNA, 21 nt, Artificial Sequence
  Description of Artificial Sequence: Synthetic oligonucleotide
ucggugcacaggcaccugcaa

SEQ ID NO: 91 - RNA, 21 nt, Artificial Sequence
  Description of Artificial Sequence: Synthetic oligonucleotide
cucuggaagguacauugccca

SEQ ID NO: 92 - RNA, 21 nt, Artificial Sequence
  Description of Artificial Sequence: Synthetic oligonucleotide
gcugcaagagagauccuccua

SEQ ID NO: 93 - RNA, 21 nt, Artificial Sequence
  Description of Artificial Sequence: Synthetic oligonucleotide
auccuccugaugcccacucca

SEQ ID NO: 94 - RNA, 21 nt, Artificial Sequence
  Description of Artificial Sequence: Synthetic oligonucleotide
cagggaagcaugguuggacaa

SEQ ID NO: 95 - RNA, 21 nt, Artificial Sequence
  Description of Artificial Sequence: Synthetic oligonucleotide
uuggacagugcuccagggaaa

SEQ ID NO: 96 - RNA, 21 nt, Artificial Sequence
  Description of Artificial Sequence: Synthetic oligonucleotide
ccagggaagaaucgggcguca

SEQ ID NO: 97 - RNA, 21 nt, Artificial Sequence
  Description of Artificial Sequence: Synthetic oligonucleotide
ccagacaacccauuucgagua

SEQ ID NO: 98 - RNA, 21 nt, Artificial Sequence
  Description of Artificial Sequence: Synthetic oligonucleotide
ucgaguggcccuggaauacaa

SEQ ID NO: 99 - RNA, 21 nt, Artificial Sequence
  Description of Artificial Sequence: Synthetic oligonucleotide
ggaauacaucuccaguggaaa

SEQ ID NO: 100 - RNA, 21 nt, Artificial Sequence
  Description of Artificial Sequence: Synthetic oligonucleotide
caguggaaaccgcagcuugua

SEQ ID NO: 101 - RNA, 21 nt, Artificial Sequence
  Description of Artificial Sequence: Synthetic oligonucleotide
cugcaguggacuucuuugcca

SEQ ID NO: 102 - RNA, 21 nt, Artificial Sequence
  Description of Artificial Sequence: Synthetic oligonucleotide
ccugaagaacugcagugaaga

SEQ ID NO: 103 - RNA, 21 nt, Artificial Sequence
  Description of Artificial Sequence: Synthetic oligonucleotide
ggcuccaagauggcccugcaa

SEQ ID NO: 104 - RNA, 21 nt, Artificial Sequence
  Description of Artificial Sequence: Synthetic oligonucleotide
cuccuucacuuguuggaauga

SEQ ID NO: 105 - RNA, 21 nt, Artificial Sequence
  Description of Artificial Sequence: Synthetic oligonucleotide
acaguccuccagcuugggcaa

SEQ ID NO: 106 - RNA, 21 nt, Artificial Sequence
  Description of Artificial Sequence: Synthetic oligonucleotide
cugugacuuccaccaggacua

SEQ ID NO: 107 - RNA, 21 nt, Artificial Sequence
  Description of Artificial Sequence: Synthetic oligonucleotide
caggacugugcccagggagaa

SEQ ID NO: 108 - RNA, 21 nt, Artificial Sequence
  Description of Artificial Sequence: Synthetic oligonucleotide
cagggagaagaugagagccaa

SEQ ID NO: 109 - RNA, 21 nt, Artificial Sequence
  Description of Artificial Sequence: Synthetic oligonucleotide
agccagaugugccggaaacua

SEQ ID NO: 110 - RNA, 21 nt, Artificial Sequence
  Description of Artificial Sequence: Synthetic oligonucleotide
cggaaacugccuguggguuua

SEQ ID NO: 111 - RNA, 21 nt, Artificial Sequence
  Description of Artificial Sequence: Synthetic oligonucleotide
ugcaacuuugaagauggcuua

SEQ ID NO: 112 - RNA, 21 nt, Artificial Sequence
  Description of Artificial Sequence: Synthetic oligonucleotide
gauggcuucuguggcuggaca

SEQ ID NO: 113 - RNA, 21 nt, Artificial Sequence
  Description of Artificial Sequence: Synthetic oligonucleotide
acccaaggcacacugucacca

SEQ ID NO: 114 - RNA, 21 nt, Artificial Sequence
  Description of Artificial Sequence: Synthetic oligonucleotide
acuccucaauggcaggucaga

SEQ ID NO: 115 - RNA, 21 nt, Artificial Sequence
  Description of Artificial Sequence: Synthetic oligonucleotide
gacccuaaaggaugcccggua

SEQ ID NO: 116 - RNA, 21 nt, Artificial Sequence
  Description of Artificial Sequence: Synthetic oligonucleotide
gcccgguuccaggaccaccaa

SEQ ID NO: 117 - RNA, 21 nt, Artificial Sequence
  Description of Artificial Sequence: Synthetic oligonucleotide
gaccaccaagaccaugcucua

SEQ ID NO: 118 - RNA, 21 nt, Artificial Sequence
  Description of Artificial Sequence: Synthetic oligonucleotide
ugcucuauugcucaguaccaa

SEQ ID NO: 119 - RNA, 21 nt, Artificial Sequence
  Description of Artificial Sequence: Synthetic oligonucleotide
gcuucugaaagugcuacagua

SEQ ID NO: 120 - RNA, 21 nt, Artificial Sequence
  Description of Artificial Sequence: Synthetic oligonucleotide
ccagugcuacguuuccugcaa

SEQ ID NO: 121 - RNA, 21 nt, Artificial Sequence
  Description of Artificial Sequence: Synthetic oligonucleotide
accgaucaagagcucuccaua

SEQ ID NO: 122 - RNA, 21 nt, Artificial Sequence
  Description of Artificial Sequence: Synthetic oligonucleotide
cucuccaugugagcuccgaaa

SEQ ID NO: 123 - RNA, 21 nt, Artificial Sequence
  Description of Artificial Sequence: Synthetic oligonucleotide
gcuccgaauguccuggcucaa

SEQ ID NO: 124 - RNA, 21 nt, Artificial Sequence
  Description of Artificial Sequence: Synthetic oligonucleotide
uggcucauucguggagucuua

SEQ ID NO: 125 - RNA, 21 nt, Artificial Sequence
  Description of Artificial Sequence: Synthetic oligonucleotide
gaaacguguccuuggugcuaa

SEQ ID NO: 126 - RNA, 21 nt, Artificial Sequence
  Description of Artificial Sequence: Synthetic oligonucleotide
gugcuaguggagaacaaaaca

SEQ ID NO: 127 - RNA, 21 nt, Artificial Sequence
  Description of Artificial Sequence: Synthetic oligonucleotide
gggaaggagcaaggcaggaua

SEQ ID NO: 128 - RNA, 21 nt, Artificial Sequence
  Description of Artificial Sequence: Synthetic oligonucleotide
ggcaggauggucuggcaugua

SEQ ID NO: 129 - RNA, 21 nt, Artificial Sequence
  Description of Artificial Sequence: Synthetic oligonucleotide
cgccuaugaaggcuugagcca

SEQ ID NO: 130 - RNA, 21 nt, Artificial Sequence
  Description of Artificial Sequence: Synthetic oligonucleotide
uugagccuguggcaguggaua

SEQ ID NO: 131 - RNA, 21 nt, Artificial Sequence
  Description of Artificial Sequence: Synthetic oligonucleotide
caguggaugguguugccucua

SEQ ID NO: 132 - RNA, 21 nt, Artificial Sequence
  Description of Artificial Sequence: Synthetic oligonucleotide
cucgaugugucugacagguua

SEQ ID NO: 133 - RNA, 21 nt, Artificial Sequence
  Description of Artificial Sequence: Synthetic oligonucleotide
cuggcugcagauggucgcaua

SEQ ID NO: 134 - RNA, 21 nt, Artificial Sequence
  Description of Artificial Sequence: Synthetic oligonucleotide
auccagagccaucguggcuua

SEQ ID NO: 135 - RNA, 21 nt, Artificial Sequence
  Description of Artificial Sequence: Synthetic oligonucleotide
guggcuuuugacaauaucuca

SEQ ID NO: 136 - RNA, 21 nt, Artificial Sequence
  Description of Artificial Sequence: Synthetic oligonucleotide
aauaucuccaucagccuggaa

SEQ ID NO: 137 - RNA, 21 nt, Artificial Sequence
  Description of Artificial Sequence: Synthetic oligonucleotide
agccuggacugcuaccucaca

SEQ ID NO: 138 - RNA, 21 nt, Artificial Sequence
  Description of Artificial Sequence: Synthetic oligonucleotide
uaccucaccauuagcggagaa

SEQ ID NO: 139 - RNA, 21 nt, Artificial Sequence
  Description of Artificial Sequence: Synthetic oligonucleotide
ggacaagauccugcagaauaa

SEQ ID NO: 140 - RNA, 21 nt, Artificial Sequence
  Description of Artificial Sequence: Synthetic oligonucleotide
agaauacagcacccaaaucaa

SEQ ID NO: 141 - RNA, 21 nt, Artificial Sequence
  Description of Artificial Sequence: Synthetic oligonucleotide
ccaaaucaagaaaccuguuua

SEQ ID NO: 142 - RNA, 21 nt, Artificial Sequence
  Description of Artificial Sequence: Synthetic oligonucleotide
ccuguuugagagaaacccaaa

SEQ ID NO: 143 - RNA, 21 nt, Artificial Sequence
  Description of Artificial Sequence: Synthetic oligonucleotide
cccaaacaaggagcugaaaca

SEQ ID NO: 144 - RNA, 21 nt, Artificial Sequence
  Description of Artificial Sequence: Synthetic oligonucleotide
gaaaauucaccaagacagaca

SEQ ID NO: 145 - RNA, 21 nt, Artificial Sequence
  Description of Artificial Sequence: Synthetic oligonucleotide
cuuugacccuacaguucauua

SEQ ID NO: 146 - RNA, 21 nt, Artificial Sequence
  Description of Artificial Sequence: Synthetic oligonucleotide
aguucauuggcuguucaccaa

SEQ ID NO: 147 - RNA, 21 nt, Artificial Sequence
  Description of Artificial Sequence: Synthetic oligonucleotide
gcacagugcaacaacgccuaa

SEQ ID NO: 148 - RNA, 21 nt, Artificial Sequence
  Description of Artificial Sequence: Synthetic oligonucleotide
cuaccagaacuccaaccugaa

SEQ ID NO: 149 - RNA, 21 nt, Artificial Sequence
  Description of Artificial Sequence: Synthetic oligonucleotide
caaccugagcguggaggugga

SEQ ID NO: 150 - RNA, 21 nt, Artificial Sequence
  Description of Artificial Sequence: Synthetic oligonucleotide
cauccagaucuggaaggugca

SEQ ID NO: 151 - RNA, 21 nt, Artificial Sequence
  Description of Artificial Sequence: Synthetic oligonucleotide
gaaggugccagccaccgacaa

SEQ ID NO: 152 - RNA, 21 nt, Artificial Sequence
  Description of Artificial Sequence: Synthetic oligonucleotide
caccgacaccuacagcaucua

SEQ ID NO: 153 - RNA, 21 nt, Artificial Sequence
  Description of Artificial Sequence: Synthetic oligonucleotide
ucucgggcuacggagcugcua

SEQ ID NO: 154 - RNA, 21 nt, Artificial Sequence
  Description of Artificial Sequence: Synthetic oligonucleotide
ggcgggaaaggcgggaagaaa

SEQ ID NO: 155 - RNA, 21 nt, Artificial Sequence
  Description of Artificial Sequence: Synthetic oligonucleotide
gggaagaacaccaugaugcga

SEQ ID NO: 156 - RNA, 21 nt, Artificial Sequence
  Description of Artificial Sequence: Synthetic oligonucleotide
gaugcggucccacggcgugua

SEQ ID NO: 157 - RNA, 21 nt, Artificial Sequence
  Description of Artificial Sequence: Synthetic oligonucleotide
ggcgugucugugcugggcaua

SEQ ID NO: 158 - RNA, 21 nt, Artificial Sequence
  Description of Artificial Sequence: Synthetic oligonucleotide
ggcaucuucaaccuggagaaa

SEQ ID NO: 159 - RNA, 21 nt, Artificial Sequence
  Description of Artificial Sequence: Synthetic oligonucleotide
aaggaugacaugcuguacaua

SEQ ID NO: 160 - RNA, 21 nt, Artificial Sequence
  Description of Artificial Sequence: Synthetic oligonucleotide
cuguacauccugguugggcaa

SEQ ID NO: 161 - RNA, 21 nt, Artificial Sequence
  Description of Artificial Sequence: Synthetic oligonucleotide
guugggcagcagggagaggaa

SEQ ID NO: 162 - RNA, 21 nt, Artificial Sequence
  Description of Artificial Sequence: Synthetic oligonucleotide
aguacaaaccaguuaauccaa

SEQ ID NO: 163 - RNA, 21 nt, Artificial Sequence
  Description of Artificial Sequence: Synthetic oligonucleotide
uuaauccagaaagucugcaua

SEQ ID NO: 164 - RNA, 21 nt, Artificial Sequence
  Description of Artificial Sequence: Synthetic oligonucleotide
ugcauuggagagaacaaugua

SEQ ID NO: 165 - RNA, 21 nt, Artificial Sequence
  Description of Artificial Sequence: Synthetic oligonucleotide
aaugugauagaagaagaaaua

SEQ ID NO: 166 - RNA, 21 nt, Artificial Sequence
  Description of Artificial Sequence: Synthetic oligonucleotide
auccgugugaacagaagcgua

SEQ ID NO: 167 - RNA, 21 nt, Artificial Sequence
  Description of Artificial Sequence: Synthetic oligonucleotide
gaagcgugcaugagugggcaa

SEQ ID NO: 168 - RNA, 21 nt, Artificial Sequence
  Description of Artificial Sequence: Synthetic oligonucleotide
caccuacguauuuaagaugaa

SEQ ID NO: 169 - RNA, 21 nt, Artificial Sequence
  Description of Artificial Sequence: Synthetic oligonucleotide
uaagaugaaggauggagugca

SEQ ID NO: 170 - RNA, 21 nt, Artificial Sequence
  Description of Artificial Sequence: Synthetic oligonucleotide
cugaucauugcagccggagga

SEQ ID NO: 171 - RNA, 21 nt, Artificial Sequence
  Description of Artificial Sequence: Synthetic oligonucleotide
ccaagacagacacguuccaca

SEQ ID NO: 172 - RNA, 21 nt, Artificial Sequence
  Description of Artificial Sequence: Synthetic oligonucleotide
agagagacuggagaauaacua

SEQ ID NO: 173 - RNA, 21 nt, Artificial Sequence
  Description of Artificial Sequence: Synthetic oligonucleotide
gaauaacuccucgguucuaga

SEQ ID NO: 174 - RNA, 21 nt, Artificial Sequence
  Description of Artificial Sequence: Synthetic oligonucleotide
agggcuaaacggcaauuccga

SEQ ID NO: 175 - RNA, 21 nt, Artificial Sequence
  Description of Artificial Sequence: Synthetic oligonucleotide
auuccggagccgcagguggua

SEQ ID NO: 176 - RNA, 21 nt, Artificial Sequence
  Description of Artificial Sequence: Synthetic oligonucleotide
gugguggagguggcuggaaua

SEQ ID NO: 177 - RNA, 21 nt, Artificial Sequence
  Description of Artificial Sequence: Synthetic oligonucleotide
uggaaugauaacacuuccuua

SEQ ID NO: 178 - RNA, 21 nt, Artificial Sequence
  Description of Artificial Sequence: Synthetic oligonucleotide
acuuccuugcucugggccgga

SEQ ID NO: 179 - RNA, 21 nt, Artificial Sequence
  Description of Artificial Sequence: Synthetic oligonucleotide
ggccggaaaaucuuugcagga

SEQ ID NO: 180 - RNA, 21 nt, Artificial Sequence
  Description of Artificial Sequence: Synthetic oligonucleotide
ugcaggagggugccaccggaa

SEQ ID NO: 181 - RNA, 21 nt, Artificial Sequence
  Description of Artificial Sequence: Synthetic oligonucleotide
ccaccggaggacauuccugca

SEQ ID NO: 182 - RNA, 21 nt, Artificial Sequence
  Description of Artificial Sequence: Synthetic oligonucleotide
aggcggaggauauauaggcga

SEQ ID NO: 183 - RNA, 21 nt, Artificial Sequence
  Description of Artificial Sequence: Synthetic oligonucleotide
augcagccucaaacaaugaca

SEQ ID NO: 184 - RNA, 21 nt, Artificial Sequence
  Description of Artificial Sequence: Synthetic oligonucleotide
guuuccuucaucaguccacua

SEQ ID NO: 185 - RNA, 21 nt, Artificial Sequence
  Description of Artificial Sequence: Synthetic oligonucleotide
cacugggcauccuguacacca

SEQ ID NO: 186 - RNA, 21 nt, Artificial Sequence
  Description of Artificial Sequence: Synthetic oligonucleotide
cagcuuuaaaagugauggaaa

SEQ ID NO: 187 - RNA, 21 nt, Artificial Sequence
  Description of Artificial Sequence: Synthetic oligonucleotide
uaagcauuaucuaaacugcaa

SEQ ID NO: 188 - RNA, 21 nt, Artificial Sequence
  Description of Artificial Sequence: Synthetic oligonucleotide
ugcagucacugugagguagaa

SEQ ID NO: 189 - RNA, 21 nt, Artificial Sequence
  Description of Artificial Sequence: Synthetic oligonucleotide
guagacgaaugucacauggaa

SEQ ID NO: 190 - RNA, 21 nt, Artificial Sequence
  Description of Artificial Sequence: Synthetic oligonucleotide
cccugaaagccacaaggucaa

SEQ ID NO: 191 - RNA, 21 nt, Artificial Sequence
  Description of Artificial Sequence: Synthetic oligonucleotide
aaggucaucugcuucugugaa

SEQ ID NO: 192 - RNA, 21 nt, Artificial Sequence
  Description of Artificial Sequence: Synthetic oligonucleotide
uucugugaccacgggacggua

SEQ ID NO: 193 - RNA, 21 nt, Artificial Sequence
  Description of Artificial Sequence: Synthetic oligonucleotide
gcuggcugaggauggcgucua

SEQ ID NO: 194 - RNA, 21 nt, Artificial Sequence
  Description of Artificial Sequence: Synthetic oligonucleotide
gucuccugcauugugucacca

SEQ ID NO: 195 - RNA, 21 nt, Artificial Sequence
  Description of Artificial Sequence: Synthetic oligonucleotide
ccacaccugccacucucgcua

SEQ ID NO: 196 - RNA, 21 nt, Artificial Sequence
  Description of Artificial Sequence: Synthetic oligonucleotide
ucgcugauccucucuguggua

SEQ ID NO: 197 - RNA, 21 nt, Artificial Sequence
  Description of Artificial Sequence: Synthetic oligonucleotide
ucuguggugaccucugcccua

SEQ ID NO: 198 - RNA, 21 nt, Artificial Sequence
  Description of Artificial Sequence: Synthetic oligonucleotide
guccuggcuuucuccggcaua

SEQ ID NO: 199 - RNA, 21 nt, Artificial Sequence
  Description of Artificial Sequence: Synthetic oligonucleotide
uccggcaucaugauuguguaa

SEQ ID NO: 200 - RNA, 21 nt, Artificial Sequence
  Description of Artificial Sequence: Synthetic oligonucleotide
uaccgccggaagcaccaggaa

SEQ ID NO: 201 - RNA, 21 nt, Artificial Sequence
  Description of Artificial Sequence: Synthetic oligonucleotide
caccaggagcugcaagccaua

SEQ ID NO: 202 - RNA, 21 nt, Artificial Sequence
  Description of Artificial Sequence: Synthetic oligonucleotide
caagccaugcagauggagcua

SEQ ID NO: 203 - RNA, 21 nt, Artificial Sequence
  Description of Artificial Sequence: Synthetic oligonucleotide
auggagcugcagagcccugaa

SEQ ID NO: 204 - RNA, 21 nt, Artificial Sequence
  Description of Artificial Sequence: Synthetic oligonucleotide
ccugaguacaagcugagcaaa

SEQ ID NO: 205 - RNA, 21 nt, Artificial Sequence
  Description of Artificial Sequence: Synthetic oligonucleotide
gcuccgcaccucgaccaucaa

SEQ ID NO: 206 - RNA, 21 nt, Artificial Sequence
  Description of Artificial Sequence: Synthetic oligonucleotide
accaucaugaccgacuacaaa

SEQ ID NO: 207 - RNA, 21 nt, Artificial Sequence
  Description of Artificial Sequence: Synthetic oligonucleotide
caacuacugcuuugcuggcaa

SEQ ID NO: 208 - RNA, 21 nt, Artificial Sequence
  Description of Artificial Sequence: Synthetic oligonucleotide
ugcuggcaagaccuccuccaa

SEQ ID NO: 209 - RNA, 21 nt, Artificial Sequence
  Description of Artificial Sequence: Synthetic oligonucleotide
uccuccaucagugaccugaaa

SEQ ID NO: 210 - RNA, 21 nt, Artificial Sequence
  Description of Artificial Sequence: Synthetic oligonucleotide
ugaaggaggugccgcggaaaa

SEQ ID NO: 211 - RNA, 21 nt, Artificial Sequence
  Description of Artificial Sequence: Synthetic oligonucleotide
gagguguaugaaggccaggua

SEQ ID NO: 212 - RNA, 21 nt, Artificial Sequence
  Description of Artificial Sequence: Synthetic oligonucleotide
gguguccggaaugcccaacga

SEQ ID NO: 213 - RNA, 21 nt, Artificial Sequence
  Description of Artificial Sequence: Synthetic oligonucleotide
gcugugaagacgcugccugaa

SEQ ID NO: 214 - RNA, 21 nt, Artificial Sequence
  Description of Artificial Sequence: Synthetic oligonucleotide
ugugcucugaacaggacgaaa

SEQ ID NO: 215 - RNA, 21 nt, Artificial Sequence
  Description of Artificial Sequence: Synthetic oligonucleotide
ggacgaacuggauuuccucaa

SEQ ID NO: 216 - RNA, 21 nt, Artificial Sequence
  Description of Artificial Sequence: Synthetic oligonucleotide
uuccucauggaagcccugaua

SEQ ID NO: 217 - RNA, 21 nt, Artificial Sequence
  Description of Artificial Sequence: Synthetic oligonucleotide
gaucaucagcaaauucaacca

SEQ ID NO: 218 - RNA, 21 nt, Artificial Sequence
  Description of Artificial Sequence: Synthetic oligonucleotide
caaccaccagaacauuguuca

SEQ ID NO: 219 - RNA, 21 nt, Artificial Sequence
  Description of Artificial Sequence: Synthetic oligonucleotide
cgguucauccugcuggagcua

SEQ ID NO: 220 - RNA, 21 nt, Artificial Sequence
  Description of Artificial Sequence: Synthetic oligonucleotide
agaccucaaguccuuccucca

SEQ ID NO: 221 - RNA, 21 nt, Artificial Sequence
  Description of Artificial Sequence: Synthetic oligonucleotide
augcuggaccuucugcacgua

SEQ ID NO: 222 - RNA, 21 nt, Artificial Sequence
  Description of Artificial Sequence: Synthetic oligonucleotide
ucgggacauugccuguggcua

SEQ ID NO: 223 - RNA, 21 nt, Artificial Sequence
  Description of Artificial Sequence: Synthetic oligonucleotide
guggcugucaguauuuggaga

SEQ ID NO: 224 - RNA, 21 nt, Artificial Sequence
  Description of Artificial Sequence: Synthetic oligonucleotide
uggaggaaaaccacuucauca

SEQ ID NO: 225 - RNA, 21 nt, Artificial Sequence
  Description of Artificial Sequence: Synthetic oligonucleotide
ccgagacauugcugccagaaa

SEQ ID NO: 226 - RNA, 21 nt, Artificial Sequence
  Description of Artificial Sequence: Synthetic oligonucleotide
ugccagaaacugccucuugaa

SEQ ID NO: 227 - RNA, 21 nt, Artificial Sequence
  Description of Artificial Sequence: Synthetic oligonucleotide
ccucuugaccuguccaggcca

SEQ ID NO: 228 - RNA, 21 nt, Artificial Sequence
  Description of Artificial Sequence: Synthetic oligonucleotide
gcccuggaagaguggccaaga

SEQ ID NO: 229 - RNA, 21 nt, Artificial Sequence
  Description of Artificial Sequence: Synthetic oligonucleotide
ggccaagauuggagacuucga

SEQ ID NO: 230 - RNA, 21 nt, Artificial Sequence
  Description of Artificial Sequence: Synthetic oligonucleotide
cuucgggauggcccgagacaa

SEQ ID NO: 231 - RNA, 21 nt, Artificial Sequence
  Description of Artificial Sequence: Synthetic oligonucleotide
ccgagacaucuacagggcgaa

SEQ ID NO: 232 - RNA, 21 nt, Artificial Sequence
  Description of Artificial Sequence: Synthetic oligonucleotide
gagcuacuauagaaagggaga

SEQ ID NO: 233 - RNA, 21 nt, Artificial Sequence
  Description of Artificial Sequence: Synthetic oligonucleotide
gggaggcugugccaugcugca

SEQ ID NO: 234 - RNA, 21 nt, Artificial Sequence
  Description of Artificial Sequence: Synthetic oligonucleotide
augcugccaguuaaguggaua

SEQ ID NO: 235 - RNA, 21 nt, Artificial Sequence
  Description of Artificial Sequence: Synthetic oligonucleotide
gaggccuucauggaaggaaua

SEQ ID NO: 236 - RNA, 21 nt, Artificial Sequence
  Description of Artificial Sequence: Synthetic oligonucleotide
gaaggaauauucacuucuaaa

SEQ ID NO: 237 - RNA, 21 nt, Artificial Sequence
  Description of Artificial Sequence: Synthetic oligonucleotide
acuucuaaaacagacacauga

SEQ ID NO: 238 - RNA, 21 nt, Artificial Sequence
  Description of Artificial Sequence: Synthetic oligonucleotide
acaugguccuuuggagugcua

SEQ ID NO: 239 - RNA, 21 nt, Artificial Sequence
  Description of Artificial Sequence: Synthetic oligonucleotide
gugcugcuaugggaaaucuua

SEQ ID NO: 240 - RNA, 21 nt, Artificial Sequence
  Description of Artificial Sequence: Synthetic oligonucleotide
gaaaucuuuucucuuggauaa

SEQ ID NO: 241 - RNA, 21 nt, Artificial Sequence
  Description of Artificial Sequence: Synthetic oligonucleotide
caaaagcaaccaggaaguuca

SEQ ID NO: 242 - RNA, 21 nt, Artificial Sequence
  Description of Artificial Sequence: Synthetic oligonucleotide
ggaaguucuggaguuugucaa

SEQ ID NO: 243 - RNA, 21 nt, Artificial Sequence
  Description of Artificial Sequence: Synthetic oligonucleotide
guuugucaccaguggaggcca

SEQ ID NO: 244 - RNA, 21 nt, Artificial Sequence
  Description of Artificial Sequence: Synthetic oligonucleotide
ccacccaagaacugcccugga

SEQ ID NO: 245 - RNA, 21 nt, Artificial Sequence
  Description of Artificial Sequence: Synthetic oligonucleotide
ugcccugggccuguauaccga

SEQ ID NO: 246 - RNA, 21 nt, Artificial Sequence
  Description of Artificial Sequence: Synthetic oligonucleotide
guauaccggauaaugacucaa

SEQ ID NO: 247 - RNA, 21 nt, Artificial Sequence
  Description of Artificial Sequence: Synthetic oligonucleotide
gcuggcaacaucagccugaaa

SEQ ID NO: 248 - RNA, 21 nt, Artificial Sequence
  Description of Artificial Sequence: Synthetic oligonucleotide
agccugaagacaggcccaaca

SEQ ID NO: 249 - RNA, 21 nt, Artificial Sequence
  Description of Artificial Sequence: Synthetic oligonucleotide
aacuuugccaucauuuuggaa

SEQ ID NO: 250 - RNA, 21 nt, Artificial Sequence
  Description of Artificial Sequence: Synthetic oligonucleotide
ggagaggauugaauacugcaa

SEQ ID NO: 251 - RNA, 21 nt, Artificial Sequence
  Description of Artificial Sequence: Synthetic oligonucleotide
acccaggacccggauguaaua

SEQ ID NO: 252 - RNA, 21 nt, Artificial Sequence
  Description of Artificial Sequence: Synthetic oligonucleotide
gauguaaucaacaccgcuuua

SEQ ID NO: 253 - RNA, 21 nt, Artificial Sequence
  Description of Artificial Sequence: Synthetic oligonucleotide
accgcuuugccgauagaauaa

SEQ ID NO: 254 - RNA, 21 nt, Artificial Sequence
  Description of Artificial Sequence: Synthetic oligonucleotide
auagaauaugguccacuugua

SEQ ID NO: 255 - RNA, 21 nt, Artificial Sequence
  Description of Artificial Sequence: Synthetic oligonucleotide
ccacuuguggaagaggaagaa

SEQ ID NO: 256 - RNA, 21 nt, Artificial Sequence
  Description of Artificial Sequence: Synthetic oligonucleotide
ggaagagaaagugccugugaa

SEQ ID NO: 257 - RNA, 21 nt, Artificial Sequence
  Description of Artificial Sequence: Synthetic oligonucleotide
ugaggcccaaggacccugaga

SEQ ID NO: 258 - RNA, 21 nt, Artificial Sequence
  Description of Artificial Sequence: Synthetic oligonucleotide
ccuccucuccuggucucucaa

SEQ ID NO: 259 - RNA, 21 nt, Artificial Sequence
  Description of Artificial Sequence: Synthetic oligonucleotide
ucucucaacaggcaaaacgga

SEQ ID NO: 260 - RNA, 21 nt, Artificial Sequence
  Description of Artificial Sequence: Synthetic oligonucleotide
ucugccuaccaccuccucuga

SEQ ID NO: 261 - RNA, 21 nt, Artificial Sequence
  Description of Artificial Sequence: Synthetic oligonucleotide
cuccucuggcaaggcugcaaa

SEQ ID NO: 262 - RNA, 21 nt, Artificial Sequence
  Description of Artificial Sequence: Synthetic oligonucleotide
cugcaaagaaacccacagcua

SEQ ID NO: 263 - RNA, 21 nt, Artificial Sequence
  Description of Artificial Sequence: Synthetic oligonucleotide
gcagagaucucuguucgagua

SEQ ID NO: 264 - RNA, 21 nt, Artificial Sequence
  Description of Artificial Sequence: Synthetic oligonucleotide
guucgagucccuagagggcca

SEQ ID NO: 265 - RNA, 21 nt, Artificial Sequence
  Description of Artificial Sequence: Synthetic oligonucleotide
gugaauauggcauucucucaa

SEQ ID NO: 266 - RNA, 21 nt, Artificial Sequence
  Description of Artificial Sequence: Synthetic oligonucleotide
cucucaguccaacccuccuua

SEQ ID NO: 267 - RNA, 21 nt, Artificial Sequence
  Description of Artificial Sequence: Synthetic oligonucleotide
ccuccuucggaguugcacaaa

SEQ ID NO: 268 - RNA, 21 nt, Artificial Sequence
  Description of Artificial Sequence: Synthetic oligonucleotide
ccacggauccagaaacaagca

SEQ ID NO: 269 - RNA, 21 nt, Artificial Sequence
  Description of Artificial Sequence: Synthetic oligonucleotide
caccagcuuguggaacccaaa

SEQ ID NO: 270 - RNA, 21 nt, Artificial Sequence
  Description of Artificial Sequence: Synthetic oligonucleotide
gaacccaacguacggcuccua

SEQ ID NO: 271 - RNA, 21 nt, Artificial Sequence
  Description of Artificial Sequence: Synthetic oligonucleotide
uccugguuuacagagaaacca

SEQ ID NO: 272 - RNA, 21 nt, Artificial Sequence
  Description of Artificial Sequence: Synthetic oligonucleotide
uaauccuauagcaaagaagga

SEQ ID NO: 273 - RNA, 21 nt, Artificial Sequence
  Description of Artificial Sequence: Synthetic oligonucleotide
agaaggagccacacgacagga

SEQ ID NO: 274 - RNA, 21 nt, Artificial Sequence
  Description of Artificial Sequence: Synthetic oligonucleotide
agggaagcuguacugucccaa

SEQ ID NO: 275 - RNA, 21 nt, Artificial Sequence
  Description of Artificial Sequence: Synthetic oligonucleotide
ccaccuaacguugcaacugga

SEQ ID NO: 276 - RNA, 21 nt, Artificial Sequence
  Description of Artificial Sequence: Synthetic oligonucleotide
acugcuccuagagcccucuua

SEQ ID NO: 277 - RNA, 21 nt, Artificial Sequence
  Description of Artificial Sequence: Synthetic oligonucleotide
ucgcugacugccaauaugaaa

SEQ ID NO: 278 - RNA, 21 nt, Artificial Sequence
  Description of Artificial Sequence: Synthetic oligonucleotide
aauaugaaggagguaccucua

SEQ ID NO: 279 - RNA, 21 nt, Artificial Sequence
  Description of Artificial Sequence: Synthetic oligonucleotide
guaccucuguucaggcuacga

SEQ ID NO: 280 - RNA, 21 nt, Artificial Sequence
  Description of Artificial Sequence: Synthetic oligonucleotide
gcuacgucacuucccuuguga

SEQ ID NO: 281 - RNA, 21 nt, Artificial Sequence
  Description of Artificial Sequence: Synthetic oligonucleotide
uugugggaaugucaauuacga

SEQ ID NO: 282 - RNA, 21 nt, Artificial Sequence
  Description of Artificial Sequence: Synthetic oligonucleotide
ggcuaccagcaacagggcuua

SEQ ID NO: 283 - RNA, 21 nt, Artificial Sequence
  Description of Artificial Sequence: Synthetic oligonucleotide
uugcccuuagaagccgcuaca

SEQ ID NO: 284 - RNA, 21 nt, Artificial Sequence
  Description of Artificial Sequence: Synthetic oligonucleotide
uggagcuggucauuacgagga

SEQ ID NO: 285 - RNA, 21 nt, Artificial Sequence
  Description of Artificial Sequence: Synthetic oligonucleotide
uuacgaggauaccauucugaa

SEQ ID NO: 286 - RNA, 21 nt, Artificial Sequence
  Description of Artificial Sequence: Synthetic oligonucleotide
cauucugaaaagcaagaauaa

SEQ ID NO: 287 - RNA, 21 nt, Artificial Sequence
  Description of Artificial Sequence: Synthetic oligonucleotide
agaauagcaugaaccagccua

SEQ ID NO: 288 - RNA, 21 nt, Artificial Sequence
  Description of Artificial Sequence: Synthetic oligonucleotide
gagcucggucgcacacucaca

SEQ ID NO: 289 - RNA, 21 nt, Artificial Sequence
  Description of Artificial Sequence: Synthetic oligonucleotide
acacucacuucucuuccuuga

SEQ ID NO: 290 - RNA, 21 nt, Artificial Sequence
  Description of Artificial Sequence: Synthetic oligonucleotide
gggaucccuaagaccguggaa

SEQ ID NO: 291 - RNA, 21 nt, Artificial Sequence
  Description of Artificial Sequence: Synthetic oligonucleotide
accguggaggagagagaggca

SEQ ID NO: 292 - RNA, 21 nt, Artificial Sequence
  Description of Artificial Sequence: Synthetic oligonucleotide
agagaggcaauggcuccuuca

SEQ ID NO: 293 - RNA, 21 nt, Artificial Sequence
  Description of Artificial Sequence: Synthetic oligonucleotide
gcuccuucacaaaccagagaa

SEQ ID NO: 294 - RNA, 21 nt, Artificial Sequence
  Description of Artificial Sequence: Synthetic oligonucleotide
gagaccaaaugucacguuuua

SEQ ID NO: 295 - RNA, 21 nt, Artificial Sequence
  Description of Artificial Sequence: Synthetic oligonucleotide
cacguuuuguuuugugccaaa

SEQ ID NO: 296 - RNA, 21 nt, Artificial Sequence
  Description of Artificial Sequence: Synthetic oligonucleotide
gccaaccuauuuugaaguaca

SEQ ID NO: 297 - RNA, 21 nt, Artificial Sequence
  Description of Artificial Sequence: Synthetic oligonucleotide
uguauuuugaaaaugcuuuaa

SEQ ID NO: 298 - RNA, 21 nt, Artificial Sequence
  Description of Artificial Sequence: Synthetic oligonucleotide
uuuagaaagguuuugagcaua

SEQ ID NO: 299 - RNA, 21 nt, Artificial Sequence
  Description of Artificial Sequence: Synthetic oligonucleotide
uugagcauggguucauccuaa

SEQ ID NO: 300 - RNA, 21 nt, Artificial Sequence
  Description of Artificial Sequence: Synthetic oligonucleotide
cauccuauucuuucgaaagaa

SEQ ID NO: 301 - RNA, 21 nt, Artificial Sequence
  Description of Artificial Sequence: Synthetic oligonucleotide
aaugagugauaaauacaagga

SEQ ID NO: 302 - RNA, 21 nt, Artificial Sequence
  Description of Artificial Sequence: Synthetic oligonucleotide
auacaaggcccagauguggua

SEQ ID NO: 303 - RNA, 21 nt, Artificial Sequence
  Description of Artificial Sequence: Synthetic oligonucleotide
gaugugguugcauaagguuua

SEQ ID NO: 304 - RNA, 21 nt, Artificial Sequence
  Description of Artificial Sequence: Synthetic oligonucleotide
ugcauguuuguuguauacuua

SEQ ID NO: 305 - RNA, 21 nt, Artificial Sequence
  Description of Artificial Sequence: Synthetic oligonucleotide
uuccuuaugcuucuuucaaaa

SEQ ID NO: 306 - RNA, 21 nt, Artificial Sequence
  Description of Artificial Sequence: Synthetic oligonucleotide
uuucaaauugugugugcucua

SEQ ID NO: 307 - RNA, 21 nt, Artificial Sequence
  Description of Artificial Sequence: Synthetic oligonucleotide
cugcuucaauguagucagaaa

SEQ ID NO: 308 - RNA, 21 nt, Artificial Sequence
  Description of Artificial Sequence: Synthetic oligonucleotide
agucagaauuagcugcuucua

SEQ ID NO: 309 - RNA, 21 nt, Artificial Sequence
  Description of Artificial Sequence: Synthetic oligonucleotide
ugcuucuauguuucauaguua

SEQ ID NO: 310 - RNA, 21 nt, Artificial Sequence
  Description of Artificial Sequence: Synthetic oligonucleotide
auguuuccuugccuuguugaa

SEQ ID NO: 311 - RNA, 21 nt, Artificial Sequence
  Description of Artificial Sequence: Synthetic oligonucleotide
uguggacaugagccauuugaa

SEQ ID NO: 312 - RNA, 21 nt, Artificial Sequence
  Description of Artificial Sequence: Synthetic oligonucleotide
acggaaauaaaggaguuauua

SEQ ID NO: 313 - RNA, 21 nt, Artificial Sequence
  Description of Artificial Sequence: Synthetic oligonucleotide
aguuauuuguaaugacuaaaa

SEQ ID NO: 314 - RNA, 23 nt, Artificial Sequence
  Description of Artificial Sequence: Synthetic oligonucleotide
uagccgcuggaucgcaucugccu

SEQ ID NO: 315 - RNA, 23 nt, Artificial Sequence
  Description of Artificial Sequence: Synthetic oligonucleotide
ugguaccagcugcuaccaccgcu

SEQ ID NO: 316 - RNA, 23 nt, Artificial Sequence
  Description of Artificial Sequence: Synthetic oligonucleotide
uuccucacccaucaucagcgccc

SEQ ID NO: 317 - RNA, 23 nt, Artificial Sequence
  Description of Artificial Sequence: Synthetic oligonucleotide
ucuccucagaguucgcaggcacu

SEQ ID NO: 318 - RNA, 23 nt, Artificial Sequence
  Description of Artificial Sequence: Synthetic oligonucleotide
uugcgcaaguuugcagcguccuu

SEQ ID NO: 319 - RNA, 23 nt, Artificial Sequence
  Description of Artificial Sequence: Synthetic oligonucleotide
uuucugggcgugaaucccagccc

SEQ ID NO: 320 - RNA, 23 nt, Artificial Sequence
  Description of Artificial Sequence: Synthetic oligonucleotide
uugccugcugaacuucugggcgu

SEQ ID NO: 321 - RNA, 23 nt, Artificial Sequence
  Description of Artificial Sequence: Synthetic oligonucleotide
ugaaggcuucggacugucugccu

SEQ ID NO: 322 - RNA, 23 nt, Artificial Sequence
  Description of Artificial Sequence: Synthetic oligonucleotide
ucucaagcuaucucuccgcugcg

SEQ ID NO: 323 - RNA, 23 nt, Artificial Sequence
  Description of Artificial Sequence: Synthetic oligonucleotide
uugccgucuugcgcacccucaag

SEQ ID NO: 324 - RNA, 23 nt, Artificial Sequence
  Description of Artificial Sequence: Synthetic oligonucleotide
uuccuccaagcucuucugcccgg

SEQ ID NO: 325 - RNA, 23 nt, Artificial Sequence
  Description of Artificial Sequence: Synthetic oligonucleotide
uuuuugcguuccuuuuggcuccu

SEQ ID NO: 326 - RNA, 23 nt, Artificial Sequence
  Description of Artificial Sequence: Synthetic oligonucleotide
uacgcuguccuggccgccuuuug

SEQ ID NO: 327 - RNA, 23 nt, Artificial Sequence
  Description of Artificial Sequence: Synthetic oligonucleotide
uuuuaaggcugagaacggcggcu

SEQ ID NO: 328 - RNA, 23 nt, Artificial Sequence
  Description of Artificial Sequence: Synthetic oligonucleotide
uaaucucugcaacuuuuaaggcu

SEQ ID NO: 329 - RNA, 23 nt, Artificial Sequence
  Description of Artificial Sequence: Synthetic oligonucleotide
ugguggcaguuggguaccguccu

SEQ ID NO: 330 - RNA, 23 nt, Artificial Sequence
  Description of Artificial Sequence: Synthetic oligonucleotide
uugguugaagggagguggcaguu

SEQ ID NO: 331 - RNA, 23 nt, Artificial Sequence
  Description of Artificial Sequence: Synthetic oligonucleotide
uagaggaacuacuaugguugaag

SEQ ID NO: 332 - RNA, 23 nt, Artificial Sequence
  Description of Artificial Sequence: Synthetic oligonucleotide
uugcgcucgguacagaggaacua

SEQ ID NO: 333 - RNA, 23 nt, Artificial Sequence
  Description of Artificial Sequence: Synthetic oligonucleotide
uucuguagcucgcugcgcucggu

SEQ ID NO: 334 - RNA, 23 nt, Artificial Sequence
  Description of Artificial Sequence: Synthetic oligonucleotide
uacuggcugggaccuugagccuc

SEQ ID NO: 335 - RNA, 23 nt, Artificial Sequence
  Description of Artificial Sequence: Synthetic oligonucleotide
uagcacacugggcucacuggcug

SEQ ID NO: 336 - RNA, 23 nt, Artificial Sequence
  Description of Artificial Sequence: Synthetic oligonucleotide
ucagagacacucaagcacacugg

SEQ ID NO: 337 - RNA, 23 nt, Artificial Sequence
  Description of Artificial Sequence: Synthetic oligonucleotide
ugagucuaaaugaaacagaccug

SEQ ID NO: 338 - RNA, 23 nt, Artificial Sequence
  Description of Artificial Sequence: Synthetic oligonucleotide
uaacugcacggaggcgagcagga

SEQ ID NO: 339 - RNA, 23 nt, Artificial Sequence
  Description of Artificial Sequence: Synthetic oligonucleotide
ucgcgcaagucucuugcuuuccc

SEQ ID NO: 340 - RNA, 23 nt, Artificial Sequence
  Description of Artificial Sequence: Synthetic oligonucleotide
uagaggacugugcgugcgcgcaa

SEQ ID NO: 341 - RNA, 23 nt, Artificial Sequence
  Description of Artificial Sequence: Synthetic oligonucleotide
uuccaccugaucuccagaggacu

SEQ ID NO: 342 - RNA, 23 nt, Artificial Sequence
  Description of Artificial Sequence: Synthetic oligonucleotide
ucuugguacccagcggcuccuuc

SEQ ID NO: 343 - RNA, 23 nt, Artificial Sequence
  Description of Artificial Sequence: Synthetic oligonucleotide
ucucugaacaguccuugguaccc

SEQ ID NO: 344 - RNA, 23 nt, Artificial Sequence
  Description of Artificial Sequence: Synthetic oligonucleotide
ugagaugggaagaggcucugaac

SEQ ID NO: 345 - RNA, 23 nt, Artificial Sequence
  Description of Artificial Sequence: Synthetic oligonucleotide
uccguuuacacugcucuccgggc

SEQ ID NO: 346 - RNA, 23 nt, Artificial Sequence
  Description of Artificial Sequence: Synthetic oligonucleotide
uacaggagcccgauggcucccau

SEQ ID NO: 347 - RNA, 23 nt, Artificial Sequence
  Description of Artificial Sequence: Synthetic oligonucleotide
uagcugccguggaaagcagcagc

SEQ ID NO: 348 - RNA, 23 nt, Artificial Sequence
  Description of Artificial Sequence: Synthetic oligonucleotide
uaggcgcgaguagcugaguggcu

SEQ ID NO: 349 - RNA, 23 nt, Artificial Sequence
  Description of Artificial Sequence: Synthetic oligonucleotide
uacugccagacucuuccucugca

SEQ ID NO: 350 - RNA, 23 nt, Artificial Sequence
  Description of Artificial Sequence: Synthetic oligonucleotide
uaccacgaagucaacugccagac

SEQ ID NO: 351 - RNA, 23 nt, Artificial Sequence
  Description of Artificial Sequence: Synthetic oligonucleotide
uaagagcgagggcaccacgaagu

SEQ ID NO: 352 - RNA, 23 nt, Artificial Sequence
  Description of Artificial Sequence: Synthetic oligonucleotide
ugcguagacacggaagagcgagg

SEQ ID NO: 353 - RNA, 23 nt, Artificial Sequence
  Description of Artificial Sequence: Synthetic oligonucleotide
uaguaggucccgggcguagacac

SEQ ID NO: 354 - RNA, 23 nt, Artificial Sequence
  Description of Artificial Sequence: Synthetic oligonucleotide
ugaugguggcagcaguagguccc

SEQ ID NO: 355 - RNA, 23 nt, Artificial Sequence
  Description of Artificial Sequence: Synthetic oligonucleotide
uugccagccuucagcuccgagga

SEQ ID NO: 356 - RNA, 23 nt, Artificial Sequence
  Description of Artificial Sequence: Synthetic oligonucleotide
uccuucagcacccuggacagcgu

SEQ ID NO: 357 - RNA, 23 nt, Artificial Sequence
  Description of Artificial Sequence: Synthetic oligonucleotide
uagcaccaacugcuuggcacgcc

SEQ ID NO: 358 - RNA, 23 nt, Artificial Sequence
  Description of Artificial Sequence: Synthetic oligonucleotide
uucgcccagcuccagcaccaacu

SEQ ID NO: 359 - RNA, 23 nt, Artificial Sequence
  Description of Artificial Sequence: Synthetic oligonucleotide
ucaacccuccaagaucgccuccu

SEQ ID NO: 360 - RNA, 23 nt, Artificial Sequence
  Description of Artificial Sequence: Synthetic oligonucleotide
ucugagauugaacuggagcagcc

SEQ ID NO: 361 - RNA, 23 nt, Artificial Sequence
  Description of Artificial Sequence: Synthetic oligonucleotide
uaccaacugaacagcucgcugag

SEQ ID NO: 362 - RNA, 23 nt, Artificial Sequence
  Description of Artificial Sequence: Synthetic oligonucleotide
ucgccuuggcgaauccaccaacu

SEQ ID NO: 363 - RNA, 23 nt, Artificial Sequence
  Description of Artificial Sequence: Synthetic oligonucleotide
uaggcggauccucagucgcccuu

SEQ ID NO: 364 - RNA, 23 nt, Artificial Sequence
  Description of Artificial Sequence: Synthetic oligonucleotide
uuucucgggcaucaggcggaucc

SEQ ID NO: 365 - RNA, 23 nt, Artificial Sequence
  Description of Artificial Sequence: Synthetic oligonucleotide
uuuccgacgccuucuucucgggc

SEQ ID NO: 366 - RNA, 23 nt, Artificial Sequence
  Description of Artificial Sequence: Synthetic oligonucleotide
uacagccuucccucucugcccac

SEQ ID NO: 367 - RNA, 23 nt, Artificial Sequence
  Description of Artificial Sequence: Synthetic oligonucleotide
ugcgaauugccgcggacagccuu

SEQ ID NO: 368 - RNA, 23 nt, Artificial Sequence
  Description of Artificial Sequence: Synthetic oligonucleotide
ucgaagaucuggaagagaaggcg

SEQ ID NO: 369 - RNA, 23 nt, Artificial Sequence
  Description of Artificial Sequence: Synthetic oligonucleotide
uuccaaggagcuaugaccagucc

SEQ ID NO: 370 - RNA, 23 nt, Artificial Sequence
  Description of Artificial Sequence: Synthetic oligonucleotide
uguuuguuggugauuccaaggag

SEQ ID NO: 371 - RNA, 23 nt, Artificial Sequence
  Description of Artificial Sequence: Synthetic oligonucleotide
uagaaggagaaggcauguuuguu

SEQ ID NO: 372 - RNA, 23 nt, Artificial Sequence
  Description of Artificial Sequence: Synthetic oligonucleotide
uuguaaaauaaucaggagaagga

SEQ ID NO: 373 - RNA, 23 nt, Artificial Sequence
  Description of Artificial Sequence: Synthetic oligonucleotide
uaggugagauuccauguaaaaua

SEQ ID NO: 374 - RNA, 23 nt, Artificial Sequence
  Description of Artificial Sequence: Synthetic oligonucleotide
ucuuucauuauccaggugagauu

SEQ ID NO: 375 - RNA, 23 nt, Artificial Sequence
  Description of Artificial Sequence: Synthetic oligonucleotide
uaaagggaaggagucuuucauua

SEQ ID NO: 376 - RNA, 23 nt, Artificial Sequence
  Description of Artificial Sequence: Synthetic oligonucleotide
ucgaugagacaggaaagggaagg

SEQ ID NO: 377 - RNA, 23 nt, Artificial Sequence
  Description of Artificial Sequence: Synthetic oligonucleotide
uagaccauaucggcugcgaugag

SEQ ID NO: 378 - RNA, 23 nt, Artificial Sequence
  Description of Artificial Sequence: Synthetic oligonucleotide
uggaagucaaagcugcacuccag

SEQ ID NO: 379 - RNA, 23 nt, Artificial Sequence
  Description of Artificial Sequence: Synthetic oligonucleotide
uuuccugaggucaugcaguggag

SEQ ID NO: 380 - RNA, 23 nt, Artificial Sequence
  Description of Artificial Sequence: Synthetic oligonucleotide
uaggaccagcucugguuccugag

SEQ ID NO: 381 - RNA, 23 nt, Artificial Sequence
  Description of Artificial Sequence: Synthetic oligonucleotide
uuccagcaaguccaucugggagg

SEQ ID NO: 382 - RNA, 23 nt, Artificial Sequence
  Description of Artificial Sequence: Synthetic oligonucleotide
ugcaucuccuuagaacgcucugc

SEQ ID NO: 383 - RNA, 23 nt, Artificial Sequence
  Description of Artificial Sequence: Synthetic oligonucleotide
uagaaaggagccucugggcaucu

SEQ ID NO: 384 - RNA, 23 nt, Artificial Sequence
  Description of Artificial Sequence: Synthetic oligonucleotide
uguguugagaaggagaaaggagc

SEQ ID NO: 385 - RNA, 23 nt, Artificial Sequence
  Description of Artificial Sequence: Synthetic oligonucleotide
uuggugugcuuggagucagcuga

SEQ ID NO: 386 - RNA, 23 nt, Artificial Sequence
  Description of Artificial Sequence: Synthetic oligonucleotide
uacggacucaggauggugugcuu

SEQ ID NO: 387 - RNA, 23 nt, Artificial Sequence
  Description of Artificial Sequence: Synthetic oligonucleotide
uugcuccucauccacggacucag

SEQ ID NO: 388 - RNA, 23 nt, Artificial Sequence
  Description of Artificial Sequence: Synthetic oligonucleotide
ugugugcagugcucacugcugcu

SEQ ID NO: 389 - RNA, 23 nt, Artificial Sequence
  Description of Artificial Sequence: Synthetic oligonucleotide
uaccgagacggccagugugcagu

SEQ ID NO: 390 - RNA, 23 nt, Artificial Sequence
  Description of Artificial Sequence: Synthetic oligonucleotide
uugcaggugccugugcaccgaga

SEQ ID NO: 391 - RNA, 23 nt, Artificial Sequence
  Description of Artificial Sequence: Synthetic oligonucleotide
ugggcaauguaccuuccagaggg

SEQ ID NO: 392 - RNA, 23 nt, Artificial Sequence
  Description of Artificial Sequence: Synthetic oligonucleotide
uaggaggaucucucuugcagccu

SEQ ID NO: 393 - RNA, 23 nt, Artificial Sequence
  Description of Artificial Sequence: Synthetic oligonucleotide
uggagugggcaucaggaggaucu

SEQ ID NO: 394 - RNA, 23 nt, Artificial Sequence
  Description of Artificial Sequence: Synthetic oligonucleotide
uuguccaaccaugcuucccugga

SEQ ID NO: 395 - RNA, 23 nt, Artificial Sequence
  Description of Artificial Sequence: Synthetic oligonucleotide
uuucccuggagcacuguccaacc

SEQ ID NO: 396 - RNA, 23 nt, Artificial Sequence
  Description of Artificial Sequence: Synthetic oligonucleotide
ugacgcccgauucuucccuggag

SEQ ID NO: 397 - RNA, 23 nt, Artificial Sequence
  Description of Artificial Sequence: Synthetic oligonucleotide
uacucgaaauggguugucuggac

SEQ ID NO: 398 - RNA, 23 nt, Artificial Sequence
  Description of Artificial Sequence: Synthetic oligonucleotide
uuguauuccagggccacucgaaa

SEQ ID NO: 399 - RNA, 23 nt, Artificial Sequence
  Description of Artificial Sequence: Synthetic oligonucleotide
uuuccacuggagauguauuccag

SEQ ID NO: 400 - RNA, 23 nt, Artificial Sequence
  Description of Artificial Sequence: Synthetic oligonucleotide
uacaagcugcgguuuccacugga

SEQ ID NO: 401 - RNA, 23 nt, Artificial Sequence
  Description of Artificial Sequence: Synthetic oligonucleotide
uggcaaagaaguccacugcagac

SEQ ID NO: 402 - RNA, 23 nt, Artificial Sequence
  Description of Artificial Sequence: Synthetic oligonucleotide
ucuucacugcaguucuucagggc

SEQ ID NO: 403 - RNA, 23 nt, Artificial Sequence
  Description of Artificial Sequence: Synthetic oligonucleotide
uugcagggccaucuuggagccug

SEQ ID NO: 404 - RNA, 23 nt, Artificial Sequence
  Description of Artificial Sequence: Synthetic oligonucleotide
ucauuccaacaagugaaggagcu

SEQ ID NO: 405 - RNA, 23 nt, Artificial Sequence
  Description of Artificial Sequence: Synthetic oligonucleotide
uugcccaagcuggaggacugucc

SEQ ID NO: 406 - RNA, 23 nt, Artificial Sequence
  Description of Artificial Sequence: Synthetic oligonucleotide
uaguccugguggaagucacaggc

SEQ ID NO: 407 - RNA, 23 nt, Artificial Sequence
  Description of Artificial Sequence: Synthetic oligonucleotide
uucucccugggcacaguccuggu

SEQ ID NO: 408 - RNA, 23 nt, Artificial Sequence
  Description of Artificial Sequence: Synthetic oligonucleotide
uuggcucucaucuucucccuggg

SEQ ID NO: 409 - RNA, 23 nt, Artificial Sequence
  Description of Artificial Sequence: Synthetic oligonucleotide
uaguuuccggcacaucuggcucu

SEQ ID NO: 410 - RNA, 23 nt, Artificial Sequence
  Description of Artificial Sequence: Synthetic oligonucleotide
uaaacccacaggcaguuuccggc

SEQ ID NO: 411 - RNA, 23 nt, Artificial Sequence
  Description of Artificial Sequence: Synthetic oligonucleotide
uaagccaucuucaaaguugcagu

SEQ ID NO: 412 - RNA, 23 nt, Artificial Sequence
  Description of Artificial Sequence: Synthetic oligonucleotide
uguccagccacagaagccaucuu

SEQ ID NO: 413 - RNA, 23 nt, Artificial Sequence
  Description of Artificial Sequence: Synthetic oligonucleotide
uggugacagugugccuugggucc

SEQ ID NO: 414 - RNA, 23 nt, Artificial Sequence
  Description of Artificial Sequence: Synthetic oligonucleotide
ucugaccugccauugaggagugu

SEQ ID NO: 415 - RNA, 23 nt, Artificial Sequence
  Description of Artificial Sequence: Synthetic oligonucleotide
uaccgggcauccuuuaggguccu

SEQ ID NO: 416 - RNA, 23 nt, Artificial Sequence
  Description of Artificial Sequence: Synthetic oligonucleotide
uuggugguccuggaaccgggcau

SEQ ID NO: 417 - RNA, 23 nt, Artificial Sequence
  Description of Artificial Sequence: Synthetic oligonucleotide
uagagcauggucuuggugguccu

SEQ ID NO: 418 - RNA, 23 nt, Artificial Sequence
  Description of Artificial Sequence: Synthetic oligonucleotide
uugguacugagcaauagagcaug

SEQ ID NO: 419 - RNA, 23 nt, Artificial Sequence
  Description of Artificial Sequence: Synthetic oligonucleotide
uacuguagcacuuucagaagcgg

SEQ ID NO: 420 - RNA, 23 nt, Artificial Sequence
  Description of Artificial Sequence: Synthetic oligonucleotide
uugcaggaaacguagcacugguc

SEQ ID NO: 421 - RNA, 23 nt, Artificial Sequence
  Description of Artificial Sequence: Synthetic oligonucleotide
uauggagagcucuugaucggugc

SEQ ID NO: 422 - RNA, 23 nt, Artificial Sequence
  Description of Artificial Sequence: Synthetic oligonucleotide
uuucggagcucacauggagagcu

SEQ ID NO: 423 - RNA, 23 nt, Artificial Sequence
  Description of Artificial Sequence: Synthetic oligonucleotide
uugagccaggacauucggagcuc

SEQ ID NO: 424 - RNA, 23 nt, Artificial Sequence
  Description of Artificial Sequence: Synthetic oligonucleotide
uaagacuccacgaaugagccagg

SEQ ID NO: 425 - RNA, 23 nt, Artificial Sequence
  Description of Artificial Sequence: Synthetic oligonucleotide
uuagcaccaaggacacguuuccc

SEQ ID NO: 426 - RNA, 23 nt, Artificial Sequence
  Description of Artificial Sequence: Synthetic oligonucleotide
uguuuuguucuccacuagcacca

SEQ ID NO: 427 - RNA, 23 nt, Artificial Sequence
  Description of Artificial Sequence: Synthetic oligonucleotide
uauccugccuugcuccuucccgg

SEQ ID NO: 428 - RNA, 23 nt, Artificial Sequence
  Description of Artificial Sequence: Synthetic oligonucleotide
uacaugccagaccauccugccuu

SEQ ID NO: 429 - RNA, 23 nt, Artificial Sequence
  Description of Artificial Sequence: Synthetic oligonucleotide
uggcucaagccuucauaggcggc

SEQ ID NO: 430 - RNA, 23 nt, Artificial Sequence
  Description of Artificial Sequence: Synthetic oligonucleotide
uauccacugccacaggcucaagc

SEQ ID NO: 431 - RNA, 23 nt, Artificial Sequence
  Description of Artificial Sequence: Synthetic oligonucleotide
uagaggcaacaccauccacugcc

SEQ ID NO: 432 - RNA, 23 nt, Artificial Sequence
  Description of Artificial Sequence: Synthetic oligonucleotide
uaaccugucagacacaucgagga

SEQ ID NO: 433 - RNA, 23 nt, Artificial Sequence
  Description of Artificial Sequence: Synthetic oligonucleotide
uaugcgaccaucugcagccagaa

SEQ ID NO: 434 - RNA, 23 nt, Artificial Sequence
  Description of Artificial Sequence: Synthetic oligonucleotide
uaagccacgauggcucuggaucc

SEQ ID NO: 435 - RNA, 23 nt, Artificial Sequence
  Description of Artificial Sequence: Synthetic oligonucleotide
ugagauauugucaaaagccacga

SEQ ID NO: 436 - RNA, 23 nt, Artificial Sequence
  Description of Artificial Sequence: Synthetic oligonucleotide
uuccaggcugauggagauauugu

SEQ ID NO: 437 - RNA, 23 nt, Artificial Sequence
  Description of Artificial Sequence: Synthetic oligonucleotide
ugugagguagcaguccaggcuga

SEQ ID NO: 438 - RNA, 23 nt, Artificial Sequence
  Description of Artificial Sequence: Synthetic oligonucleotide
uucuccgcuaauggugagguagc

SEQ ID NO: 439 - RNA, 23 nt, Artificial Sequence
  Description of Artificial Sequence: Synthetic oligonucleotide
uuauucugcaggaucuuguccuc

SEQ ID NO: 440 - RNA, 23 nt, Artificial Sequence
  Description of Artificial Sequence: Synthetic oligonucleotide
uugauuugggugcuguauucugc

SEQ ID NO: 441 - RNA, 23 nt, Artificial Sequence
  Description of Artificial Sequence: Synthetic oligonucleotide
uaaacagguuucuugauuugggu

SEQ ID NO: 442 - RNA, 23 nt, Artificial Sequence
  Description of Artificial Sequence: Synthetic oligonucleotide
uuuggguuucucucaaacagguu

SEQ ID NO: 443 - RNA, 23 nt, Artificial Sequence
  Description of Artificial Sequence: Synthetic oligonucleotide
uguuucagcuccuuguuuggguu

SEQ ID NO: 444 - RNA, 23 nt, Artificial Sequence
  Description of Artificial Sequence: Synthetic oligonucleotide
ugucugucuuggugaauuuuccc

SEQ ID NO: 445 - RNA, 23 nt, Artificial Sequence
  Description of Artificial Sequence: Synthetic oligonucleotide
uaaugaacuguagggucaaagau

SEQ ID NO: 446 - RNA, 23 nt, Artificial Sequence
  Description of Artificial Sequence: Synthetic oligonucleotide
uuggugaacagccaaugaacugu

SEQ ID NO: 447 - RNA, 23 nt, Artificial Sequence
  Description of Artificial Sequence: Synthetic oligonucleotide
uuaggcguuguugcacugugccu

SEQ ID NO: 448 - RNA, 23 nt, Artificial Sequence
  Description of Artificial Sequence: Synthetic oligonucleotide
uucagguuggaguucugguaggc

SEQ ID NO: 449 - RNA, 23 nt, Artificial Sequence
  Description of Artificial Sequence: Synthetic oligonucleotide
uccaccuccacgcucagguugga

SEQ ID NO: 450 - RNA, 23 nt, Artificial Sequence
  Description of Artificial Sequence: Synthetic oligonucleotide
ugcaccuuccagaucuggaugcc

SEQ ID NO: 451 - RNA, 23 nt, Artificial Sequence
  Description of Artificial Sequence: Synthetic oligonucleotide
uugucgguggcuggcaccuucca

SEQ ID NO: 452 - RNA, 23 nt, Artificial Sequence
  Description of Artificial Sequence: Synthetic oligonucleotide
uagaugcuguaggugucgguggc

SEQ ID NO: 453 - RNA, 23 nt, Artificial Sequence
  Description of Artificial Sequence: Synthetic oligonucleotide
uagcagcuccguagcccgagaug

SEQ ID NO: 454 - RNA, 23 nt, Artificial Sequence
  Description of Artificial Sequence: Synthetic oligonucleotide
uuucuucccgccuuucccgccag

SEQ ID NO: 455 - RNA, 23 nt, Artificial Sequence
  Description of Artificial Sequence: Synthetic oligonucleotide
ucgcaucaugguguucuucccgc

SEQ ID NO: 456 - RNA, 23 nt, Artificial Sequence
  Description of Artificial Sequence: Synthetic oligonucleotide
uacacgccgugggaccgcaucau

SEQ ID NO: 457 - RNA, 23 nt, Artificial Sequence
  Description of Artificial Sequence: Synthetic oligonucleotide
uaugcccagcacagacacgccgu

SEQ ID NO: 458 - RNA, 23 nt, Artificial Sequence
  Description of Artificial Sequence: Synthetic oligonucleotide
uuucuccagguugaagaugccca

SEQ ID NO: 459 - RNA, 23 nt, Artificial Sequence
  Description of Artificial Sequence: Synthetic oligonucleotide
uauguacagcaugucauccuucu

SEQ ID NO: 460 - RNA, 23 nt, Artificial Sequence
  Description of Artificial Sequence: Synthetic oligonucleotide
uugcccaaccaggauguacagca

SEQ ID NO: 461 - RNA, 23 nt, Artificial Sequence
  Description of Artificial Sequence: Synthetic oligonucleotide
uuccucucccugcugcccaacca

SEQ ID NO: 462 - RNA, 23 nt, Artificial Sequence
  Description of Artificial Sequence: Synthetic oligonucleotide
uuggauuaacugguuuguacugg

SEQ ID NO: 463 - RNA, 23 nt, Artificial Sequence
  Description of Artificial Sequence: Synthetic oligonucleotide
uaugcagacuuucuggauuaacu

SEQ ID NO: 464 - RNA, 23 nt, Artificial Sequence
  Description of Artificial Sequence: Synthetic oligonucleotide
uacauuguucucuccaaugcaga

SEQ ID NO: 465 - RNA, 23 nt, Artificial Sequence
  Description of Artificial Sequence: Synthetic oligonucleotide
uauuucuucuucuaucacauugu

SEQ ID NO: 466 - RNA, 23 nt, Artificial Sequence
  Description of Artificial Sequence: Synthetic oligonucleotide
uacgcuucuguucacacggauuu

SEQ ID NO: 467 - RNA, 23 nt, Artificial Sequence
  Description of Artificial Sequence: Synthetic oligonucleotide
uugcccacucaugcacgcuucug

SEQ ID NO: 468 - RNA, 23 nt, Artificial Sequence
  Description of Artificial Sequence: Synthetic oligonucleotide
uucaucuuaaauacguagguggc

SEQ ID NO: 469 - RNA, 23 nt, Artificial Sequence
  Description of Artificial Sequence: Synthetic oligonucleotide
ugcacuccauccuucaucuuaaa

SEQ ID NO: 470 - RNA, 23 nt, Artificial Sequence
  Description of Artificial Sequence: Synthetic oligonucleotide
uccuccggcugcaaugaucaggg

SEQ ID NO: 471 - RNA, 23 nt, Artificial Sequence
  Description of Artificial Sequence: Synthetic oligonucleotide
uguggaacgugucugucuuggcc

SEQ ID NO: 472 - RNA, 23 nt, Artificial Sequence
  Description of Artificial Sequence: Synthetic oligonucleotide
uaguuauucuccagucucucugg

SEQ ID NO: 473 - RNA, 23 nt, Artificial Sequence
  Description of Artificial Sequence: Synthetic oligonucleotide
ucuagaaccgaggaguuauucuc

SEQ ID NO: 474 - RNA, 23 nt, Artificial Sequence
  Description of Artificial Sequence: Synthetic oligonucleotide
ucggaauugccguuuagcccuag

SEQ ID NO: 475 - RNA, 23 nt, Artificial Sequence
  Description of Artificial Sequence: Synthetic oligonucleotide
uaccaccugcggcuccggaauug

SEQ ID NO: 476 - RNA, 23 nt, Artificial Sequence
  Description of Artificial Sequence: Synthetic oligonucleotide
uauuccagccaccuccaccaccu

SEQ ID NO: 477 - RNA, 23 nt, Artificial Sequence
  Description of Artificial Sequence: Synthetic oligonucleotide
uaaggaaguguuaucauuccagc

SEQ ID NO: 478 - RNA, 23 nt, Artificial Sequence
  Description of Artificial Sequence: Synthetic oligonucleotide
uccggcccagagcaaggaagugu

SEQ ID NO: 479 - RNA, 23 nt, Artificial Sequence
  Description of Artificial Sequence: Synthetic oligonucleotide
uccugcaaagauuuuccggccca

SEQ ID NO: 480 - RNA, 23 nt, Artificial Sequence
  Description of Artificial Sequence: Synthetic oligonucleotide
uuccgguggcacccuccugcaaa

SEQ ID NO: 481 - RNA, 23 nt, Artificial Sequence
  Description of Artificial Sequence: Synthetic oligonucleotide
ugcaggaauguccuccgguggca

SEQ ID NO: 482 - RNA, 23 nt, Artificial Sequence
  Description of Artificial Sequence: Synthetic oligonucleotide
ucgccuauauauccuccgccucc

SEQ ID NO: 483 - RNA, 23 nt, Artificial Sequence
  Description of Artificial Sequence: Synthetic oligonucleotide
ugucauuguuugaggcugcauug

SEQ ID NO: 484 - RNA, 23 nt, Artificial Sequence
  Description of Artificial Sequence: Synthetic oligonucleotide
uaguggacugaugaaggaaaccc

SEQ ID NO: 485 - RNA, 23 nt, Artificial Sequence
  Description of Artificial Sequence: Synthetic oligonucleotide
ugguguacaggaugcccagugga

SEQ ID NO: 486 - RNA, 23 nt, Artificial Sequence
  Description of Artificial Sequence: Synthetic oligonucleotide
uuuccaucacuuuuaaagcuggg

SEQ ID NO: 487 - RNA, 23 nt, Artificial Sequence
  Description of Artificial Sequence: Synthetic oligonucleotide
uugcaguuuagauaaugcuuaau

SEQ ID NO: 488 - RNA, 23 nt, Artificial Sequence
  Description of Artificial Sequence: Synthetic oligonucleotide
uucuaccucacagugacugcagu

SEQ ID NO: 489 - RNA, 23 nt, Artificial Sequence
  Description of Artificial Sequence: Synthetic oligonucleotide
uuccaugugacauucgucuaccu

SEQ ID NO: 490 - RNA, 23 nt, Artificial Sequence
  Description of Artificial Sequence: Synthetic oligonucleotide
uugaccuuguggcuuucaggguc

SEQ ID NO: 491 - RNA, 23 nt, Artificial Sequence
  Description of Artificial Sequence: Synthetic oligonucleotide
uucacagaagcagaugaccuugu

SEQ ID NO: 492 - RNA, 23 nt, Artificial Sequence
  Description of Artificial Sequence: Synthetic oligonucleotide
uaccgucccguggucacagaagc

SEQ ID NO: 493 - RNA, 23 nt, Artificial Sequence
  Description of Artificial Sequence: Synthetic oligonucleotide
uagacgccauccucagccagcac

SEQ ID NO: 494 - RNA, 23 nt, Artificial Sequence
  Description of Artificial Sequence: Synthetic oligonucleotide
uggugacacaaugcaggagacgc

SEQ ID NO: 495 - RNA, 23 nt, Artificial Sequence
  Description of Artificial Sequence: Synthetic oligonucleotide
uagcgagaguggcagguguggcu

SEQ ID NO: 496 - RNA, 23 nt, Artificial Sequence
  Description of Artificial Sequence: Synthetic oligonucleotide
uaccacagagaggaucagcgaga

SEQ ID NO: 497 - RNA, 23 nt, Artificial Sequence
  Description of Artificial Sequence: Synthetic oligonucleotide
uagggcagaggucaccacagaga

SEQ ID NO: 498 - RNA, 23 nt, Artificial Sequence
  Description of Artificial Sequence: Synthetic oligonucleotide
uaugccggagaaagccaggacca

SEQ ID NO: 499 - RNA, 23 nt, Artificial Sequence
  Description of Artificial Sequence: Synthetic oligonucleotide
uuacacaaucaugaugccggaga

SEQ ID NO: 500 - RNA, 23 nt, Artificial Sequence
  Description of Artificial Sequence: Synthetic oligonucleotide
uuccuggugcuuccggcgguaca

SEQ ID NO: 501 - RNA, 23 nt, Artificial Sequence
  Description of Artificial Sequence: Synthetic oligonucleotide
uauggcuugcagcuccuggugcu

SEQ ID NO: 502 - RNA, 23 nt, Artificial Sequence
  Description of Artificial Sequence: Synthetic oligonucleotide
uagcuccaucugcauggcuugca

SEQ ID NO: 503 - RNA, 23 nt, Artificial Sequence
  Description of Artificial Sequence: Synthetic oligonucleotide
uucagggcucugcagcuccaucu

SEQ ID NO: 504 - RNA, 23 nt, Artificial Sequence
  Description of Artificial Sequence: Synthetic oligonucleotide
uuugcucagcuuguacucagggc

SEQ ID NO: 505 - RNA, 23 nt, Artificial Sequence
  Description of Artificial Sequence: Synthetic oligonucleotide
uugauggucgaggugcggagcuu

SEQ ID NO: 506 - RNA, 23 nt, Artificial Sequence
  Description of Artificial Sequence: Synthetic oligonucleotide
uuuguagucggucaugauggucg

SEQ ID NO: 507 - RNA, 23 nt, Artificial Sequence
  Description of Artificial Sequence: Synthetic oligonucleotide
uugccagcaaagcaguaguuggg

SEQ ID NO: 508 - RNA, 23 nt, Artificial Sequence
  Description of Artificial Sequence: Synthetic oligonucleotide
uuggaggaggucuugccagcaaa

SEQ ID NO: 509 - RNA, 23 nt, Artificial Sequence
  Description of Artificial Sequence: Synthetic oligonucleotide
uuucaggucacugauggaggagg

SEQ ID NO: 510 - RNA, 23 nt, Artificial Sequence
  Description of Artificial Sequence: Synthetic oligonucleotide
uuuuccgcggcaccuccuucagg

SEQ ID NO: 511 - RNA, 23 nt, Artificial Sequence
  Description of Artificial Sequence: Synthetic oligonucleotide
uaccuggccuucauacaccuccc

SEQ ID NO: 512 - RNA, 23 nt, Artificial Sequence
  Description of Artificial Sequence: Synthetic oligonucleotide
ucguugggcauuccggacaccug

SEQ ID NO: 513 - RNA, 23 nt, Artificial Sequence
  Description of Artificial Sequence: Synthetic oligonucleotide
uucaggcagcgucuucacagcca

SEQ ID NO: 514 - RNA, 23 nt, Artificial Sequence
  Description of Artificial Sequence: Synthetic oligonucleotide
uuucguccuguucagagcacacu

SEQ ID NO: 515 - RNA, 23 nt, Artificial Sequence
  Description of Artificial Sequence: Synthetic oligonucleotide
uugaggaaauccaguucguccug

SEQ ID NO: 516 - RNA, 23 nt, Artificial Sequence
  Description of Artificial Sequence: Synthetic oligonucleotide
uaucagggcuuccaugaggaaau

SEQ ID NO: 517 - RNA, 23 nt, Artificial Sequence
  Description of Artificial Sequence: Synthetic oligonucleotide
ugguugaauuugcugaugaucag

SEQ ID NO: 518 - RNA, 23 nt, Artificial Sequence
  Description of Artificial Sequence: Synthetic oligonucleotide
ugaacaauguucuggugguugaa

SEQ ID NO: 519 - RNA, 23 nt, Artificial Sequence
  Description of Artificial Sequence: Synthetic oligonucleotide
uagcuccagcaggaugaaccggg

SEQ ID NO: 520 - RNA, 23 nt, Artificial Sequence
  Description of Artificial Sequence: Synthetic oligonucleotide
uggaggaaggacuugaggucucc

SEQ ID NO: 521 - RNA, 23 nt, Artificial Sequence
  Description of Artificial Sequence: Synthetic oligonucleotide
uacgugcagaagguccagcaugg

SEQ ID NO: 522 - RNA, 23 nt, Artificial Sequence
  Description of Artificial Sequence: Synthetic oligonucleotide
uagccacaggcaaugucccgagc

SEQ ID NO: 523 - RNA, 23 nt, Artificial Sequence
  Description of Artificial Sequence: Synthetic oligonucleotide
ucuccaaauacugacagccacag

SEQ ID NO: 524 - RNA, 23 nt, Artificial Sequence
  Description of Artificial Sequence: Synthetic oligonucleotide
ugaugaagugguuuuccuccaaa

SEQ ID NO: 525 - RNA, 23 nt, Artificial Sequence
  Description of Artificial Sequence: Synthetic oligonucleotide
uuucuggcagcaaugucucggug

SEQ ID NO: 526 - RNA, 23 nt, Artificial Sequence
  Description of Artificial Sequence: Synthetic oligonucleotide
uucaagaggcaguuucuggcagc

SEQ ID NO: 527 - RNA, 23 nt, Artificial Sequence
  Description of Artificial Sequence: Synthetic oligonucleotide
uggccuggacaggucaagaggca

SEQ ID NO: 528 - RNA, 23 nt, Artificial Sequence
  Description of Artificial Sequence: Synthetic oligonucleotide
ucuuggccacucuuccagggccu

SEQ ID NO: 529 - RNA, 23 nt, Artificial Sequence
  Description of Artificial Sequence: Synthetic oligonucleotide
ucgaagucuccaaucuuggccac

SEQ ID NO: 530 - RNA, 23 nt, Artificial Sequence
  Description of Artificial Sequence: Synthetic oligonucleotide
uugucucgggccaucccgaaguc

SEQ ID NO: 531 - RNA, 23 nt, Artificial Sequence
  Description of Artificial Sequence: Synthetic oligonucleotide
uucgcccuguagaugucucgggc

SEQ ID NO: 532 - RNA, 23 nt, Artificial Sequence
  Description of Artificial Sequence: Synthetic oligonucleotide
ucucccuuucuauaguagcucgc

SEQ ID NO: 533 - RNA, 23 nt, Artificial Sequence
  Description of Artificial Sequence: Synthetic oligonucleotide
ugcagcauggcacagccucccuu

SEQ ID NO: 534 - RNA, 23 nt, Artificial Sequence
  Description of Artificial Sequence: Synthetic oligonucleotide
uauccacuuaacuggcagcaugg

SEQ ID NO: 535 - RNA, 23 nt, Artificial Sequence
  Description of Artificial Sequence: Synthetic oligonucleotide
uauuccuuccaugaaggccucug

SEQ ID NO: 536 - RNA, 23 nt, Artificial Sequence
  Description of Artificial Sequence: Synthetic oligonucleotide
uuuagaagugaauauuccuucca

SEQ ID NO: 537 - RNA, 23 nt, Artificial Sequence
  Description of Artificial Sequence: Synthetic oligonucleotide
ucaugugucuguuuuagaaguga

SEQ ID NO: 538 - RNA, 23 nt, Artificial Sequence
  Description of Artificial Sequence: Synthetic oligonucleotide
uagcacuccaaaggaccaugugu

SEQ ID NO: 539 - RNA, 23 nt, Artificial Sequence
  Description of Artificial Sequence: Synthetic oligonucleotide
uaagauuucccauagcagcacuc

SEQ ID NO: 540 - RNA, 23 nt, Artificial Sequence
  Description of Artificial Sequence: Synthetic oligonucleotide
uuauccaagagaaaagauuuccc

SEQ ID NO: 541 - RNA, 23 nt, Artificial Sequence
  Description of Artificial Sequence: Synthetic oligonucleotide
ugaacuuccugguugcuuuugcu

SEQ ID NO: 542 - RNA, 23 nt, Artificial Sequence
  Description of Artificial Sequence: Synthetic oligonucleotide
uugacaaacuccagaacuuccug

SEQ ID NO: 543 - RNA, 23 nt, Artificial Sequence
  Description of Artificial Sequence: Synthetic oligonucleotide
uggccuccacuggugacaaacuc

SEQ ID NO: 544 - RNA, 23 nt, Artificial Sequence
  Description of Artificial Sequence: Synthetic oligonucleotide
uccagggcaguucuugggugggu

SEQ ID NO: 545 - RNA, 23 nt, Artificial Sequence
  Description of Artificial Sequence: Synthetic oligonucleotide
ucgguauacaggcccagggcagu

SEQ ID NO: 546 - RNA, 23 nt, Artificial Sequence
  Description of Artificial Sequence: Synthetic oligonucleotide
uugagucauuauccgguauacag

SEQ ID NO: 547 - RNA, 23 nt, Artificial Sequence
  Description of Artificial Sequence: Synthetic oligonucleotide
uuucaggcugauguugccagcac

SEQ ID NO: 548 - RNA, 23 nt, Artificial Sequence
  Description of Artificial Sequence: Synthetic oligonucleotide
uguugggccugucuucaggcuga

SEQ ID NO: 549 - RNA, 23 nt, Artificial Sequence
  Description of Artificial Sequence: Synthetic oligonucleotide
uuccaaaaugauggcaaaguugg

SEQ ID NO: 550 - RNA, 23 nt, Artificial Sequence
  Description of Artificial Sequence: Synthetic oligonucleotide
uugcaguauucaauccucuccaa

SEQ ID NO: 551 - RNA, 23 nt, Artificial Sequence
  Description of Artificial Sequence: Synthetic oligonucleotide
uauuacauccggguccugggugc

SEQ ID NO: 552 - RNA, 23 nt, Artificial Sequence
  Description of Artificial Sequence: Synthetic oligonucleotide
uaaagcgguguugauuacauccg

SEQ ID NO: 553 - RNA, 23 nt, Artificial Sequence
  Description of Artificial Sequence: Synthetic oligonucleotide
uuauucuaucggcaaagcggugu

SEQ ID NO: 554 - RNA, 23 nt, Artificial Sequence
  Description of Artificial Sequence: Synthetic oligonucleotide
uacaaguggaccauauucuaucg

SEQ ID NO: 555 - RNA, 23 nt, Artificial Sequence
  Description of Artificial Sequence: Synthetic oligonucleotide
uucuuccucuuccacaaguggac

SEQ ID NO: 556 - RNA, 23 nt, Artificial Sequence
  Description of Artificial Sequence: Synthetic oligonucleotide
uucacaggcacuuucucuuccuc

SEQ ID NO: 557 - RNA, 23 nt, Artificial Sequence
  Description of Artificial Sequence: Synthetic oligonucleotide
ucucaggguccuugggccucaca

SEQ ID NO: 558 - RNA, 23 nt, Artificial Sequence
  Description of Artificial Sequence: Synthetic oligonucleotide
uugagagaccaggagaggaggaa

SEQ ID NO: 559 - RNA, 23 nt, Artificial Sequence
  Description of Artificial Sequence: Synthetic oligonucleotide
uccguuuugccuguugagagacc

SEQ ID NO: 560 - RNA, 23 nt, Artificial Sequence
  Description of Artificial Sequence: Synthetic oligonucleotide
ucagaggaggugguaggcagagg

SEQ ID NO: 561 - RNA, 23 nt, Artificial Sequence
  Description of Artificial Sequence: Synthetic oligonucleotide
uuugcagccuugccagaggaggu

SEQ ID NO: 562 - RNA, 23 nt, Artificial Sequence
  Description of Artificial Sequence: Synthetic oligonucleotide
uagcuguggguuucuuugcagcc

SEQ ID NO: 563 - RNA, 23 nt, Artificial Sequence
  Description of Artificial Sequence: Synthetic oligonucleotide
uacucgaacagagaucucugcag

SEQ ID NO: 564 - RNA, 23 nt, Artificial Sequence
  Description of Artificial Sequence: Synthetic oligonucleotide
uggcccucuagggacucgaacag

SEQ ID NO: 565 - RNA, 23 nt, Artificial Sequence
  Description of Artificial Sequence: Synthetic oligonucleotide
uugagagaaugccauauucacgu

SEQ ID NO: 566 - RNA, 23 nt, Artificial Sequence
  Description of Artificial Sequence: Synthetic oligonucleotide
uaaggaggguuggacugagagaa

SEQ ID NO: 567 - RNA, 23 nt, Artificial Sequence
  Description of Artificial Sequence: Synthetic oligonucleotide
uuugugcaacuccgaaggagggu

SEQ ID NO: 568 - RNA, 23 nt, Artificial Sequence
  Description of Artificial Sequence: Synthetic oligonucleotide
ugcuuguuucuggauccguggac

SEQ ID NO: 569 - RNA, 23 nt, Artificial Sequence
  Description of Artificial Sequence: Synthetic oligonucleotide
uuuggguuccacaagcugguggg

SEQ ID NO: 570 - RNA, 23 nt, Artificial Sequence
  Description of Artificial Sequence: Synthetic oligonucleotide
uaggagccguacguuggguucca

SEQ ID NO: 571 - RNA, 23 nt, Artificial Sequence
  Description of Artificial Sequence: Synthetic oligonucleotide
ugguuucucuguaaaccaggagc

SEQ ID NO: 572 - RNA, 23 nt, Artificial Sequence
  Description of Artificial Sequence: Synthetic oligonucleotide
uccuucuuugcuauaggauuauu

SEQ ID NO: 573 - RNA, 23 nt, Artificial Sequence
  Description of Artificial Sequence: Synthetic oligonucleotide
uccugucguguggcuccuucuuu

SEQ ID NO: 574 - RNA, 23 nt, Artificial Sequence
  Description of Artificial Sequence: Synthetic oligonucleotide
uugggacaguacagcuucccucc

SEQ ID NO: 575 - RNA, 23 nt, Artificial Sequence
  Description of Artificial Sequence: Synthetic oligonucleotide
uccaguugcaacguuagguggga

SEQ ID NO: 576 - RNA, 23 nt, Artificial Sequence
  Description of Artificial Sequence: Synthetic oligonucleotide
uaagagggcucuaggagcaguga

SEQ ID NO: 577 - RNA, 23 nt, Artificial Sequence
  Description of Artificial Sequence: Synthetic oligonucleotide
uuucauauuggcagucagcgaag

SEQ ID NO: 578 - RNA, 23 nt, Artificial Sequence
  Description of Artificial Sequence: Synthetic oligonucleotide
uagagguaccuccuucauauugg

SEQ ID NO: 579 - RNA, 23 nt, Artificial Sequence
  Description of Artificial Sequence: Synthetic oligonucleotide
ucguagccugaacagagguaccu

SEQ ID NO: 580 - RNA, 23 nt, Artificial Sequence
  Description of Artificial Sequence: Synthetic oligonucleotide
ucacaagggaagugacguagccu

SEQ ID NO: 581 - RNA, 23 nt, Artificial Sequence
  Description of Artificial Sequence: Synthetic oligonucleotide
ucguaauugacauucccacaagg

SEQ ID NO: 582 - RNA, 23 nt, Artificial Sequence
  Description of Artificial Sequence: Synthetic oligonucleotide
uaagcccuguugcugguagccgu

SEQ ID NO: 583 - RNA, 23 nt, Artificial Sequence
  Description of Artificial Sequence: Synthetic oligonucleotide
uguagcggcuucuaagggcaagc

SEQ ID NO: 584 - RNA, 23 nt, Artificial Sequence
  Description of Artificial Sequence: Synthetic oligonucleotide
uccucguaaugaccagcuccagg

SEQ ID NO: 585 - RNA, 23 nt, Artificial Sequence
  Description of Artificial Sequence: Synthetic oligonucleotide
uucagaaugguauccucguaaug

SEQ ID NO: 586 - RNA, 23 nt, Artificial Sequence
  Description of Artificial Sequence: Synthetic oligonucleotide
uuauucuugcuuuucagaauggu

SEQ ID NO: 587 - RNA, 23 nt, Artificial Sequence
  Description of Artificial Sequence: Synthetic oligonucleotide
uaggcugguucaugcuauucuug

SEQ ID NO: 588 - RNA, 23 nt, Artificial Sequence
  Description of Artificial Sequence: Synthetic oligonucleotide
ugugagugugcgaccgagcucag

SEQ ID NO: 589 - RNA, 23 nt, Artificial Sequence
  Description of Artificial Sequence: Synthetic oligonucleotide
ucaaggaagagaagugagugugc

SEQ ID NO: 590 - RNA, 23 nt, Artificial Sequence
  Description of Artificial Sequence: Synthetic oligonucleotide
uuccacggucuuagggaucccaa

SEQ ID NO: 591 - RNA, 23 nt, Artificial Sequence
  Description of Artificial Sequence: Synthetic oligonucleotide
ugccucucucuccuccacggucu

SEQ ID NO: 592 - RNA, 23 nt, Artificial Sequence
  Description of Artificial Sequence: Synthetic oligonucleotide
ugaaggagccauugccucucucu

SEQ ID NO: 593 - RNA, 23 nt, Artificial Sequence
  Description of Artificial Sequence: Synthetic oligonucleotide
uucucugguuugugaaggagcca

SEQ ID NO: 594 - RNA, 23 nt, Artificial Sequence
  Description of Artificial Sequence: Synthetic oligonucleotide
uaaaacgugacauuuggucucug

SEQ ID NO: 595 - RNA, 23 nt, Artificial Sequence
  Description of Artificial Sequence: Synthetic oligonucleotide
uuuggcacaaaacaaaacgugac

SEQ ID NO: 596 - RNA, 23 nt, Artificial Sequence
  Description of Artificial Sequence: Synthetic oligonucleotide
uguacuucaaaauagguuggcac

SEQ ID NO: 597 - RNA, 23 nt, Artificial Sequence
  Description of Artificial Sequence: Synthetic oligonucleotide
uuaaagcauuuucaaaauacagc

SEQ ID NO: 598 - RNA, 23 nt, Artificial Sequence
  Description of Artificial Sequence: Synthetic oligonucleotide
uaugcucaaaaccuuucuaaagc

SEQ ID NO: 599 - RNA, 23 nt, Artificial Sequence
  Description of Artificial Sequence: Synthetic oligonucleotide
uuaggaugaacccaugcucaaaa

SEQ ID NO: 600 - RNA, 23 nt, Artificial Sequence
  Description of Artificial Sequence: Synthetic oligonucleotide
uucuuucgaaagaauaggaugaa

SEQ ID NO: 601 - RNA, 23 nt, Artificial Sequence
  Description of Artificial Sequence: Synthetic oligonucleotide
uccuuguauuuaucacucauuuu

SEQ ID NO: 602 - RNA, 23 nt, Artificial Sequence
  Description of Artificial Sequence: Synthetic oligonucleotide
uaccacaucugggccuuguauuu

SEQ ID NO: 603 - RNA, 23 nt, Artificial Sequence
  Description of Artificial Sequence: Synthetic oligonucleotide
uaaaccuuaugcaaccacaucug

SEQ ID NO: 604 - RNA, 23 nt, Artificial Sequence
  Description of Artificial Sequence: Synthetic oligonucleotide
uaaguauacaacaaacaugcaua

SEQ ID NO: 605 - RNA, 23 nt, Artificial Sequence
  Description of Artificial Sequence: Synthetic oligonucleotide
uuuugaaagaagcauaaggaagu

SEQ ID NO: 606 - RNA, 23 nt, Artificial Sequence
  Description of Artificial Sequence: Synthetic oligonucleotide
uagagcacacacaauuugaaaga

SEQ ID NO: 607 - RNA, 23 nt, Artificial Sequence
  Description of Artificial Sequence: Synthetic oligonucleotide
uuucugacuacauugaagcagag

SEQ ID NO: 608 - RNA, 23 nt, Artificial Sequence
  Description of Artificial Sequence: Synthetic oligonucleotide
uagaagcagcuaauucugacuac

SEQ ID NO: 609 - RNA, 23 nt, Artificial Sequence
  Description of Artificial Sequence: Synthetic oligonucleotide
uaacuaugaaacauagaagcagc

SEQ ID NO: 610 - RNA, 23 nt, Artificial Sequence
  Description of Artificial Sequence: Synthetic oligonucleotide
uucaacaaggcaaggaaacaucu

SEQ ID NO: 611 - RNA, 23 nt, Artificial Sequence
  Description of Artificial Sequence: Synthetic oligonucleotide
uucaaauggcucauguccacauc

SEQ ID NO: 612 - RNA, 23 nt, Artificial Sequence
  Description of Artificial Sequence: Synthetic oligonucleotide
uaauaacuccuuuauuuccguuc

SEQ ID NO: 613 - RNA, 23 nt, Artificial Sequence
  Description of Artificial Sequence: Synthetic oligonucleotide
uuuuagucauuacaaauaacucc

SEQ ID NO: 614 - RNA, 21 nt, Artificial Sequence
  Description of Artificial Sequence: Synthetic oligonucleotide
gcagaugcgauccagcggcua

SEQ ID NO: 615 - RNA, 21 nt, Artificial Sequence
  Description of Artificial Sequence: Synthetic oligonucleotide
cggugguagcagcugguacca

SEQ ID NO: 616 - RNA, 21 nt, Artificial Sequence
  Description of Artificial Sequence: Synthetic oligonucleotide
gcgcugaugaugggugaggaa

SEQ ID NO: 617 - RNA, 21 nt, Artificial Sequence
  Description of Artificial Sequence: Synthetic oligonucleotide
ugccugcgaacucugaggaga

SEQ ID NO: 618 - RNA, 21 nt, Artificial Sequence
  Description of Artificial Sequence: Synthetic oligonucleotide
ggacgcugcaaacuugcgcaa

SEQ ID NO: 619 - RNA, 21 nt, Artificial Sequence
  Description of Artificial Sequence: Synthetic oligonucleotide
gcugggauucacgcccagaaa

SEQ ID NO: 620 - RNA, 21 nt, Artificial Sequence
  Description of Artificial Sequence: Synthetic oligonucleotide
gcccagaaguucagcaggcaa

SEQ ID NO: 621 - RNA, 21 nt, Artificial Sequence
  Description of Artificial Sequence: Synthetic oligonucleotide
gcagacaguccgaagccuuca

SEQ ID NO: 622 - RNA, 21 nt, Artificial Sequence
  Description of Artificial Sequence: Synthetic oligonucleotide
cagcggagagauagcuugaga

SEQ ID NO: 623 - RNA, 21 nt, Artificial Sequence
  Description of Artificial Sequence: Synthetic oligonucleotide
ugagggugcgcaagacggcaa

SEQ ID NO: 624 - RNA, 21 nt, Artificial Sequence
  Description of Artificial Sequence: Synthetic oligonucleotide
gggcagaagagcuuggaggaa

SEQ ID NO: 625 - RNA, 21 nt, Artificial Sequence
  Description of Artificial Sequence: Synthetic oligonucleotide
gagccaaaaggaacgcaaaaa

SEQ ID NO: 626 - RNA, 21 nt, Artificial Sequence
  Description of Artificial Sequence: Synthetic oligonucleotide
aaaggcggccaggacagcgua

SEQ ID NO: 627 - RNA, 21 nt, Artificial Sequence
  Description of Artificial Sequence: Synthetic oligonucleotide
ccgccguucucagccuuaaaa

SEQ ID NO: 628 - RNA, 21 nt, Artificial Sequence
  Description of Artificial Sequence: Synthetic oligonucleotide
ccuuaaaaguugcagagauua

SEQ ID NO: 629 - RNA, 21 nt, Artificial Sequence
  Description of Artificial Sequence: Synthetic oligonucleotide
gacgguacccaacugccacca

SEQ ID NO: 630 - RNA, 21 nt, Artificial Sequence
  Description of Artificial Sequence: Synthetic oligonucleotide
cugccaccucccuucaaccaa

SEQ ID NO: 631 - RNA, 21 nt, Artificial Sequence
  Description of Artificial Sequence: Synthetic oligonucleotide
ucaaccauaguaguuccucua

SEQ ID NO: 632 - RNA, 21 nt, Artificial Sequence
  Description of Artificial Sequence: Synthetic oligonucleotide
guuccucuguaccgagcgcaa

SEQ ID NO: 633 - RNA, 21 nt, Artificial Sequence
  Description of Artificial Sequence: Synthetic oligonucleotide
cgagcgcagcgagcuacagaa

SEQ ID NO: 634 - RNA, 21 nt, Artificial Sequence
  Description of Artificial Sequence: Synthetic oligonucleotide
ggcucaaggucccagccagua

SEQ ID NO: 635 - RNA, 21 nt, Artificial Sequence
  Description of Artificial Sequence: Synthetic oligonucleotide
gccagugagcccagugugcua

SEQ ID NO: 636 - RNA, 21 nt, Artificial Sequence
  Description of Artificial Sequence: Synthetic oligonucleotide
agugugcuugagugucucuga

SEQ ID NO: 637 - RNA, 21 nt, Artificial Sequence
  Description of Artificial Sequence: Synthetic oligonucleotide
ggucuguuucauuuagacuca

SEQ ID NO: 638 - RNA, 21 nt, Artificial Sequence
  Description of Artificial Sequence: Synthetic oligonucleotide
cugcucgccuccgugcaguua

SEQ ID NO: 639 - RNA, 21 nt, Artificial Sequence
  Description of Artificial Sequence: Synthetic oligonucleotide
gaaagcaagagacuugcgcga

SEQ ID NO: 640 - RNA, 21 nt, Artificial Sequence
  Description of Artificial Sequence: Synthetic oligonucleotide
gcgcgcacgcacaguccucua

SEQ ID NO: 641 - RNA, 21 nt, Artificial Sequence
  Description of Artificial Sequence: Synthetic oligonucleotide
uccucuggagaucagguggaa

SEQ ID NO: 642 - RNA, 21 nt, Artificial Sequence
  Description of Artificial Sequence: Synthetic oligonucleotide
aggagccgcuggguaccaaga

SEQ ID NO: 643 - RNA, 21 nt, Artificial Sequence
  Description of Artificial Sequence: Synthetic oligonucleotide
guaccaaggacuguucagaga

SEQ ID NO: 644 - RNA, 21 nt, Artificial Sequence
  Description of Artificial Sequence: Synthetic oligonucleotide
ucagagccucuucccaucuca

SEQ ID NO: 645 - RNA, 21 nt, Artificial Sequence
  Description of Artificial Sequence: Synthetic oligonucleotide
ccggagagcaguguaaacgga

SEQ ID NO: 646 - RNA, 21 nt, Artificial Sequence
  Description of Artificial Sequence: Synthetic oligonucleotide
gggagccaucgggcuccugua

SEQ ID NO: 647 - RNA, 21 nt, Artificial Sequence
  Description of Artificial Sequence: Synthetic oligonucleotide
ugcugcuuuccacggcagcua

SEQ ID NO: 648 - RNA, 21 nt, Artificial Sequence
  Description of Artificial Sequence: Synthetic oligonucleotide
ccacucagcuacucgcgccua

SEQ ID NO: 649 - RNA, 21 nt, Artificial Sequence
  Description of Artificial Sequence: Synthetic oligonucleotide
cagaggaagagucuggcagua

SEQ ID NO: 650 - RNA, 21 nt, Artificial Sequence
  Description of Artificial Sequence: Synthetic oligonucleotide
cuggcaguugacuucguggua

SEQ ID NO: 651 - RNA, 21 nt, Artificial Sequence
  Description of Artificial Sequence: Synthetic oligonucleotide
uucguggugcccucgcucuua

SEQ ID NO: 652 - RNA, 21 nt, Artificial Sequence
  Description of Artificial Sequence: Synthetic oligonucleotide
ucgcucuuccgugucuacgca

SEQ ID NO: 653 - RNA, 21 nt, Artificial Sequence
  Description of Artificial Sequence: Synthetic oligonucleotide
gucuacgcccgggaccuacua

SEQ ID NO: 654 - RNA, 21 nt, Artificial Sequence
  Description of Artificial Sequence: Synthetic oligonucleotide
gaccuacugcugccaccauca

SEQ ID NO: 655 - RNA, 21 nt, Artificial Sequence
  Description of Artificial Sequence: Synthetic oligonucleotide
cucggagcugaaggcuggcaa

SEQ ID NO: 656 - RNA, 21 nt, Artificial Sequence
  Description of Artificial Sequence: Synthetic oligonucleotide
gcuguccagggugcugaagga

SEQ ID NO: 657 - RNA, 21 nt, Artificial Sequence
  Description of Artificial Sequence: Synthetic oligonucleotide
cgugccaagcaguuggugcua

SEQ ID NO: 658 - RNA, 21 nt, Artificial Sequence
  Description of Artificial Sequence: Synthetic oligonucleotide
uuggugcuggagcugggcgaa

SEQ ID NO: 659 - RNA, 21 nt, Artificial Sequence
  Description of Artificial Sequence: Synthetic oligonucleotide
gaggcgaucuuggaggguuga

SEQ ID NO: 660 - RNA, 21 nt, Artificial Sequence
  Description of Artificial Sequence: Synthetic oligonucleotide
cugcuccaguucaaucucaga

SEQ ID NO: 661 - RNA, 21 nt, Artificial Sequence
  Description of Artificial Sequence: Synthetic oligonucleotide
cagcgagcuguucaguuggua

SEQ ID NO: 662 - RNA, 21 nt, Artificial Sequence
  Description of Artificial Sequence: Synthetic oligonucleotide
uugguggauucgccaaggcga

SEQ ID NO: 663 - RNA, 21 nt, Artificial Sequence
  Description of Artificial Sequence: Synthetic oligonucleotide
gggcgacugaggauccgccua

SEQ ID NO: 664 - RNA, 21 nt, Artificial Sequence
  Description of Artificial Sequence: Synthetic oligonucleotide
auccgccugaugcccgagaaa

SEQ ID NO: 665 - RNA, 21 nt, Artificial Sequence
  Description of Artificial Sequence: Synthetic oligonucleotide
ccgagaagaaggcgucggaaa

SEQ ID NO: 666 - RNA, 21 nt, Artificial Sequence
  Description of Artificial Sequence: Synthetic oligonucleotide
gggcagagagggaaggcugua

SEQ ID NO: 667 - RNA, 21 nt, Artificial Sequence
  Description of Artificial Sequence: Synthetic oligonucleotide
ggcuguccgcggcaauucgca

SEQ ID NO: 668 - RNA, 21 nt, Artificial Sequence
  Description of Artificial Sequence: Synthetic oligonucleotide
ccuucucuuccagaucuucga

SEQ ID NO: 669 - RNA, 21 nt, Artificial Sequence
  Description of Artificial Sequence: Synthetic oligonucleotide
acuggucauagcuccuuggaa

SEQ ID NO: 670 - RNA, 21 nt, Artificial Sequence
  Description of Artificial Sequence: Synthetic oligonucleotide
ccuuggaaucaccaacaaaca

SEQ ID NO: 671 - RNA, 21 nt, Artificial Sequence
  Description of Artificial Sequence: Synthetic oligonucleotide
caaacaugccuucuccuucua

SEQ ID NO: 672 - RNA, 21 nt, Artificial Sequence
  Description of Artificial Sequence: Synthetic oligonucleotide
cuucuccugauuauuuuacaa

SEQ ID NO: 673 - RNA, 21 nt, Artificial Sequence
  Description of Artificial Sequence: Synthetic oligonucleotide
uuuuacauggaaucucaccua

SEQ ID NO: 674 - RNA, 21 nt, Artificial Sequence
  Description of Artificial Sequence: Synthetic oligonucleotide
ucucaccuggauaaugaaaga

SEQ ID NO: 675 - RNA, 21 nt, Artificial Sequence
  Description of Artificial Sequence: Synthetic oligonucleotide
augaaagacuccuucccuuua

SEQ ID NO: 676 - RNA, 21 nt, Artificial Sequence
  Description of Artificial Sequence: Synthetic oligonucleotide
uucccuuuccugucucaucga

SEQ ID NO: 677 - RNA, 21 nt, Artificial Sequence
  Description of Artificial Sequence: Synthetic oligonucleotide
caucgcagccgauauggucua

SEQ ID NO: 678 - RNA, 21 nt, Artificial Sequence
  Description of Artificial Sequence: Synthetic oligonucleotide
ggagugcagcuuugacuucca

SEQ ID NO: 679 - RNA, 21 nt, Artificial Sequence
  Description of Artificial Sequence: Synthetic oligonucleotide
ccacugcaugaccucaggaaa

SEQ ID NO: 680 - RNA, 21 nt, Artificial Sequence
  Description of Artificial Sequence: Synthetic oligonucleotide
caggaaccagagcugguccua

SEQ ID NO: 681 - RNA, 21 nt, Artificial Sequence
  Description of Artificial Sequence: Synthetic oligonucleotide
ucccagauggacuugcuggaa

SEQ ID NO: 682 - RNA, 21 nt, Artificial Sequence
  Description of Artificial Sequence: Synthetic oligonucleotide
agagcguucuaaggagaugca

SEQ ID NO: 683 - RNA, 21 nt, Artificial Sequence
  Description of Artificial Sequence: Synthetic oligonucleotide
augcccagaggcuccuuucua

SEQ ID NO: 684 - RNA, 21 nt, Artificial Sequence
  Description of Artificial Sequence: Synthetic oligonucleotide
uccuuucuccuucucaacaca

SEQ ID NO: 685 - RNA, 21 nt, Artificial Sequence
  Description of Artificial Sequence: Synthetic oligonucleotide
agcugacuccaagcacaccaa

SEQ ID NO: 686 - RNA, 21 nt, Artificial Sequence
  Description of Artificial Sequence: Synthetic oligonucleotide
gcacaccauccugaguccgua

SEQ ID NO: 687 - RNA, 21 nt, Artificial Sequence
  Description of Artificial Sequence: Synthetic oligonucleotide
gaguccguggaugaggagcaa

SEQ ID NO: 688 - RNA, 21 nt, Artificial Sequence
  Description of Artificial Sequence: Synthetic oligonucleotide
cagcagugagcacugcacaca

SEQ ID NO: 689 - RNA, 21 nt, Artificial Sequence
  Description of Artificial Sequence: Synthetic oligonucleotide
ugcacacuggccgucucggua

SEQ ID NO: 690 - RNA, 21 nt, Artificial Sequence
  Description of Artificial Sequence: Synthetic oligonucleotide
ucggugcacaggcaccugcaa

SEQ ID NO: 691 - RNA, 21 nt, Artificial Sequence
  Description of Artificial Sequence: Synthetic oligonucleotide
cucuggaagguacauugccca

SEQ ID NO: 692 - RNA, 21 nt, Artificial Sequence
  Description of Artificial Sequence: Synthetic oligonucleotide
gcugcaagagagauccuccua

SEQ ID NO: 693 - RNA, 21 nt, Artificial Sequence
  Description of Artificial Sequence: Synthetic oligonucleotide
auccuccugaugcccacucca

SEQ ID NO: 694 - RNA, 21 nt, Artificial Sequence
  Description of Artificial Sequence: Synthetic oligonucleotide
cagggaagcaugguuggacaa

SEQ ID NO: 695 - RNA, 21 nt, Artificial Sequence
  Description of Artificial Sequence: Synthetic oligonucleotide
uuggacagugcuccagggaaa

SEQ ID NO: 696 - RNA, 21 nt, Artificial Sequence
  Description of Artificial Sequence: Synthetic oligonucleotide
ccagggaagaaucgggcguca

SEQ ID NO: 697 - RNA, 21 nt, Artificial Sequence
  Description of Artificial Sequence: Synthetic oligonucleotide
ccagacaacccauuucgagua

SEQ ID NO: 698 - RNA, 21 nt, Artificial Sequence
  Description of Artificial Sequence: Synthetic oligonucleotide
ucgaguggcccuggaauacaa

SEQ ID NO: 699 - RNA, 21 nt, Artificial Sequence
  Description of Artificial Sequence: Synthetic oligonucleotide
ggaauacaucuccaguggaaa

SEQ ID NO: 700 - RNA, 21 nt, Artificial Sequence
  Description of Artificial Sequence: Synthetic oligonucleotide
caguggaaaccgcagcuugua

SEQ ID NO: 701 - RNA, 21 nt, Artificial Sequence
  Description of Artificial Sequence: Synthetic oligonucleotide
cugcaguggacuucuuugcca

SEQ ID NO: 702 - RNA, 21 nt, Artificial Sequence
  Description of Artificial Sequence: Synthetic oligonucleotide
ccugaagaacugcagugaaga

SEQ ID NO: 703 - RNA, 21 nt, Artificial Sequence
  Description of Artificial Sequence: Synthetic oligonucleotide
ggcuccaagauggcccugcaa

SEQ ID NO: 704 - RNA, 21 nt, Artificial Sequence
  Description of Artificial Sequence: Synthetic oligonucleotide
cuccuucacuuguuggaauga

SEQ ID NO: 705 - RNA, 21 nt, Artificial Sequence
  Description of Artificial Sequence: Synthetic oligonucleotide
acaguccuccagcuugggcaa

SEQ ID NO: 706 - RNA, 21 nt, Artificial Sequence
  Description of Artificial Sequence: Synthetic oligonucleotide
cugugacuuccaccaggacua

SEQ ID NO: 707 - RNA, 21 nt, Artificial Sequence
  Description of Artificial Sequence: Synthetic oligonucleotide
caggacugugcccagggagaa

SEQ ID NO: 708 - RNA, 21 nt, Artificial Sequence
  Description of Artificial Sequence: Synthetic oligonucleotide
cagggagaagaugagagccaa

SEQ ID NO: 709 - RNA, 21 nt, Artificial Sequence
  Description of Artificial Sequence: Synthetic oligonucleotide
agccagaugugccggaaacua

SEQ ID NO: 710 - RNA, 21 nt, Artificial Sequence
  Description of Artificial Sequence: Synthetic oligonucleotide
cggaaacugccuguggguuua

SEQ ID NO: 711 - RNA, 21 nt, Artificial Sequence
  Description of Artificial Sequence: Synthetic oligonucleotide
ugcaacuuugaagauggcuua

SEQ ID NO: 712 - RNA, 21 nt, Artificial Sequence
  Description of Artificial Sequence: Synthetic oligonucleotide
gauggcuucuguggcuggaca

SEQ ID NO: 713 - RNA, 21 nt, Artificial Sequence
  Description of Artificial Sequence: Synthetic oligonucleotide
acccaaggcacacugucacca

SEQ ID NO: 714 - RNA, 21 nt, Artificial Sequence
  Description of Artificial Sequence: Synthetic oligonucleotide
acuccucaauggcaggucaga

SEQ ID NO: 715 - RNA, 21 nt, Artificial Sequence
  Description of Artificial Sequence: Synthetic oligonucleotide
gacccuaaaggaugcccggua

SEQ ID NO: 716 - RNA, 21 nt, Artificial Sequence
  Description of Artificial Sequence: Synthetic oligonucleotide
gcccgguuccaggaccaccaa

SEQ ID NO: 717 - RNA, 21 nt, Artificial Sequence
  Description of Artificial Sequence: Synthetic oligonucleotide
gaccaccaagaccaugcucua

SEQ ID NO: 718 - RNA, 21 nt, Artificial Sequence
  Description of Artificial Sequence: Synthetic oligonucleotide
ugcucuauugcucaguaccaa

SEQ ID NO: 719 - RNA, 21 nt, Artificial Sequence
  Description of Artificial Sequence: Synthetic oligonucleotide
gcuucugaaagugcuacagua

SEQ ID NO: 720 - RNA, 21 nt, Artificial Sequence
  Description of Artificial Sequence: Synthetic oligonucleotide
ccagugcuacguuuccugcaa

SEQ ID NO: 721 - RNA, 21 nt, Artificial Sequence
  Description of Artificial Sequence: Synthetic oligonucleotide
accgaucaagagcucuccaua

SEQ ID NO: 722 - RNA, 21 nt, Artificial Sequence
  Description of Artificial Sequence: Synthetic oligonucleotide
cucuccaugugagcuccgaaa

SEQ ID NO: 723 - RNA, 21 nt, Artificial Sequence
  Description of Artificial Sequence: Synthetic oligonucleotide
gcuccgaauguccuggcucaa

SEQ ID NO: 724 - RNA, 21 nt, Artificial Sequence
  Description of Artificial Sequence: Synthetic oligonucleotide
uggcucauucguggagucuua

SEQ ID NO: 725 - RNA, 21 nt, Artificial Sequence
  Description of Artificial Sequence: Synthetic oligonucleotide
gaaacguguccuuggugcuaa

SEQ ID NO: 726 - RNA, 21 nt, Artificial Sequence
  Description of Artificial Sequence: Synthetic oligonucleotide
gugcuaguggagaacaaaaca

SEQ ID NO: 727 - RNA, 21 nt, Artificial Sequence
  Description of Artificial Sequence: Synthetic oligonucleotide
gggaaggagcaaggcaggaua

SEQ ID NO: 728 - RNA, 21 nt, Artificial Sequence
  Description of Artificial Sequence: Synthetic oligonucleotide
ggcaggauggucuggcaugua

SEQ ID NO: 729 - RNA, 21 nt, Artificial Sequence
  Description of Artificial Sequence: Synthetic oligonucleotide
cgccuaugaaggcuugagcca

SEQ ID NO: 730 - RNA, 21 nt, Artificial Sequence
  Description of Artificial Sequence: Synthetic oligonucleotide
uugagccuguggcaguggaua

SEQ ID NO: 731 - RNA, 21 nt, Artificial Sequence
  Description of Artificial Sequence: Synthetic oligonucleotide
caguggaugguguugccucua

SEQ ID NO: 732 - RNA, 21 nt, Artificial Sequence
  Description of Artificial Sequence: Synthetic oligonucleotide
cucgaugugucugacagguua

SEQ ID NO: 733 - RNA, 21 nt, Artificial Sequence
  Description of Artificial Sequence: Synthetic oligonucleotide
cuggcugcagauggucgcaua

SEQ ID NO: 734 - RNA, 21 nt, Artificial Sequence
  Description of Artificial Sequence: Synthetic oligonucleotide
auccagagccaucguggcuua

SEQ ID NO: 735 - RNA, 21 nt, Artificial Sequence
  Description of Artificial Sequence: Synthetic oligonucleotide
guggcuuuugacaauaucuca

SEQ ID NO: 736 - RNA, 21 nt, Artificial Sequence
  Description of Artificial Sequence: Synthetic oligonucleotide
aauaucuccaucagccuggaa

SEQ ID NO: 737 - RNA, 21 nt, Artificial Sequence
  Description of Artificial Sequence: Synthetic oligonucleotide
agccuggacugcuaccucaca

SEQ ID NO: 738 - RNA, 21 nt, Artificial Sequence
  Description of Artificial Sequence: Synthetic oligonucleotide
uaccucaccauuagcggagaa

SEQ ID NO: 739 - RNA, 21 nt, Artificial Sequence
  Description of Artificial Sequence: Synthetic oligonucleotide
ggacaagauccugcagaauaa

SEQ ID NO: 740 - RNA, 21 nt, Artificial Sequence
  Description of Artificial Sequence: Synthetic oligonucleotide
agaauacagcacccaaaucaa

SEQ ID NO: 741 - RNA, 21 nt, Artificial Sequence
  Description of Artificial Sequence: Synthetic oligonucleotide
ccaaaucaagaaaccuguuua

SEQ ID NO: 742 - RNA, 21 nt, Artificial Sequence
  Description of Artificial Sequence: Synthetic oligonucleotide
ccuguuugagagaaacccaaa

SEQ ID NO: 743 - RNA, 21 nt, Artificial Sequence
  Description of Artificial Sequence: Synthetic oligonucleotide
cccaaacaaggagcugaaaca

SEQ ID NO: 744 - RNA, 21 nt, Artificial Sequence
  Description of Artificial Sequence: Synthetic oligonucleotide
gaaaauucaccaagacagaca

SEQ ID NO: 745 - RNA, 21 nt, Artificial Sequence
  Description of Artificial Sequence: Synthetic oligonucleotide
cuuugacccuacaguucauua

SEQ ID NO: 746 - RNA, 21 nt, Artificial Sequence
  Description of Artificial Sequence: Synthetic oligonucleotide
aguucauuggcuguucaccaa

SEQ ID NO: 747 - RNA, 21 nt, Artificial Sequence
  Description of Artificial Sequence: Synthetic oligonucleotide
gcacagugcaacaacgccuaa

SEQ ID NO: 748 - RNA, 21 nt, Artificial Sequence
  Description of Artificial Sequence: Synthetic oligonucleotide
cuaccagaacuccaaccugaa

SEQ ID NO: 749 - RNA, 21 nt, Artificial Sequence
  Description of Artificial Sequence: Synthetic oligonucleotide
caaccugagcguggaggugga

SEQ ID NO: 750 - RNA, 21 nt, Artificial Sequence
  Description of Artificial Sequence: Synthetic oligonucleotide
cauccagaucuggaaggugca

SEQ ID NO: 751 - RNA, 21 nt, Artificial Sequence
  Description of Artificial Sequence: Synthetic oligonucleotide
gaaggugccagccaccgacaa

SEQ ID NO: 752 - RNA, 21 nt, Artificial Sequence
  Description of Artificial Sequence: Synthetic oligonucleotide
caccgacaccuacagcaucua

SEQ ID NO: 753 - RNA, 21 nt, Artificial Sequence
  Description of Artificial Sequence: Synthetic oligonucleotide
ucucgggcuacggagcugcua

SEQ ID NO: 754 - RNA, 21 nt, Artificial Sequence
  Description of Artificial Sequence: Synthetic oligonucleotide
ggcgggaaaggcgggaagaaa

SEQ ID NO: 755 - RNA, 21 nt, Artificial Sequence
  Description of Artificial Sequence: Synthetic oligonucleotide
gggaagaacaccaugaugcga

SEQ ID NO: 756 - RNA, 21 nt, Artificial Sequence
  Description of Artificial Sequence: Synthetic oligonucleotide
gaugcggucccacggcgugua

SEQ ID NO: 757 - RNA, 21 nt, Artificial Sequence
  Description of Artificial Sequence: Synthetic oligonucleotide
ggcgugucugugcugggcaua

SEQ ID NO: 758 - RNA, 21 nt, Artificial Sequence
  Description of Artificial Sequence: Synthetic oligonucleotide
ggcaucuucaaccuggagaaa

SEQ ID NO: 759 - RNA, 21 nt, Artificial Sequence
  Description of Artificial Sequence: Synthetic oligonucleotide
aaggaugacaugcuguacaua

SEQ ID NO: 760 - RNA, 21 nt, Artificial Sequence
  Description of Artificial Sequence: Synthetic oligonucleotide
cuguacauccugguugggcaa

SEQ ID NO: 761 - RNA, 21 nt, Artificial Sequence
  Description of Artificial Sequence: Synthetic oligonucleotide
guugggcagcagggagaggaa

SEQ ID NO: 762 - RNA, 21 nt, Artificial Sequence
  Description of Artificial Sequence: Synthetic oligonucleotide
aguacaaaccaguuaauccaa

SEQ ID NO: 763 - RNA, 21 nt, Artificial Sequence
  Description of Artificial Sequence: Synthetic oligonucleotide
uuaauccagaaagucugcaua

SEQ ID NO: 764 - RNA, 21 nt, Artificial Sequence
  Description of Artificial Sequence: Synthetic oligonucleotide
ugcauuggagagaacaaugua

SEQ ID NO: 765 - RNA, 21 nt, Artificial Sequence
  Description of Artificial Sequence: Synthetic oligonucleotide
aaugugauagaagaagaaaua

SEQ ID NO: 766 - RNA, 21 nt, Artificial Sequence
  Description of Artificial Sequence: Synthetic oligonucleotide
auccgugugaacagaagcgua

SEQ ID NO: 767 - RNA, 21 nt, Artificial Sequence
  Description of Artificial Sequence: Synthetic oligonucleotide
gaagcgugcaugagugggcaa

SEQ ID NO: 768 - RNA, 21 nt, Artificial Sequence
  Description of Artificial Sequence: Synthetic oligonucleotide
caccuacguauuuaagaugaa

SEQ ID NO: 769 - RNA, 21 nt, Artificial Sequence
  Description of Artificial Sequence: Synthetic oligonucleotide
uaagaugaaggauggagugca

SEQ ID NO: 770 - RNA, 21 nt, Artificial Sequence
  Description of Artificial Sequence: Synthetic oligonucleotide
cugaucauugcagccggagga

SEQ ID NO: 771 - RNA, 21 nt, Artificial Sequence
  Description of Artificial Sequence: Synthetic oligonucleotide
ccaagacagacacguuccaca

SEQ ID NO: 772 - RNA, 21 nt, Artificial Sequence
  Description of Artificial Sequence: Synthetic oligonucleotide
agagagacuggagaauaacua

SEQ ID NO: 773 - RNA, 21 nt, Artificial Sequence
  Description of Artificial Sequence: Synthetic oligonucleotide
gaauaacuccucgguucuaga

SEQ ID NO: 774 - RNA, 21 nt, Artificial Sequence
  Description of Artificial Sequence: Synthetic oligonucleotide
agggcuaaacggcaauuccga

SEQ ID NO: 775 - RNA, 21 nt, Artificial Sequence
  Description of Artificial Sequence: Synthetic oligonucleotide
auuccggagccgcagguggua

SEQ ID NO: 776 - RNA, 21 nt, Artificial Sequence
  Description of Artificial Sequence: Synthetic oligonucleotide
gugguggagguggcuggaaua

SEQ ID NO: 777 - RNA, 21 nt, Artificial Sequence
  Description of Artificial Sequence: Synthetic oligonucleotide
uggaaugauaacacuuccuua

SEQ ID NO: 778 - RNA, 21 nt, Artificial Sequence
  Description of Artificial Sequence: Synthetic oligonucleotide
acuuccuugcucugggccgga

SEQ ID NO: 779 - RNA, 21 nt, Artificial Sequence
  Description of Artificial Sequence: Synthetic oligonucleotide
ggccggaaaaucuuugcagga

SEQ ID NO: 780 - RNA, 21 nt, Artificial Sequence
  Description of Artificial Sequence: Synthetic oligonucleotide
ugcaggagggugccaccggaa

SEQ ID NO: 781 - RNA, 21 nt, Artificial Sequence
  Description of Artificial Sequence: Synthetic oligonucleotide
ccaccggaggacauuccugca

SEQ ID NO: 782 - RNA, 21 nt, Artificial Sequence
  Description of Artificial Sequence: Synthetic oligonucleotide
aggcggaggauauauaggcga

SEQ ID NO: 783 - RNA, 21 nt, Artificial Sequence
  Description of Artificial Sequence: Synthetic oligonucleotide
augcagccucaaacaaugaca

SEQ ID NO: 784 - RNA, 21 nt, Artificial Sequence
  Description of Artificial Sequence: Synthetic oligonucleotide
guuuccuucaucaguccacua

SEQ ID NO: 785 - RNA, 21 nt, Artificial Sequence
  Description of Artificial Sequence: Synthetic oligonucleotide
cacugggcauccuguacacca

SEQ ID NO: 786 - RNA, 21 nt, Artificial Sequence
  Description of Artificial Sequence: Synthetic oligonucleotide
cagcuuuaaaagugauggaaa

SEQ ID NO: 787 - RNA, 21 nt, Artificial Sequence
  Description of Artificial Sequence: Synthetic oligonucleotide
uaagcauuaucuaaacugcaa

SEQ ID NO: 788 - RNA, 21 nt, Artificial Sequence
  Description of Artificial Sequence: Synthetic oligonucleotide
ugcagucacugugagguagaa

SEQ ID NO: 789 - RNA, 21 nt, Artificial Sequence
  Description of Artificial Sequence: Synthetic oligonucleotide
guagacgaaugucacauggaa

SEQ ID NO: 790 - RNA, 21 nt, Artificial Sequence
  Description of Artificial Sequence: Synthetic oligonucleotide
cccugaaagccacaaggucaa

SEQ ID NO: 791 - RNA, 21 nt, Artificial Sequence
  Description of Artificial Sequence: Synthetic oligonucleotide
aaggucaucugcuucugugaa

SEQ ID NO: 792 - RNA, 21 nt, Artificial Sequence
  Description of Artificial Sequence: Synthetic oligonucleotide
uucugugaccacgggacggua

SEQ ID NO: 793 - RNA, 21 nt, Artificial Sequence
  Description of Artificial Sequence: Synthetic oligonucleotide
gcuggcugaggauggcgucua

SEQ ID NO: 794 - RNA, 21 nt, Artificial Sequence
  Description of Artificial Sequence: Synthetic oligonucleotide
gucuccugcauugugucacca

SEQ ID NO: 795 - RNA, 21 nt, Artificial Sequence
  Description of Artificial Sequence: Synthetic oligonucleotide
ccacaccugccacucucgcua

SEQ ID NO: 796 - RNA, 21 nt, Artificial Sequence
  Description of Artificial Sequence: Synthetic oligonucleotide
ucgcugauccucucuguggua

SEQ ID NO: 797 - RNA, 21 nt, Artificial Sequence
  Description of Artificial Sequence: Synthetic oligonucleotide
ucuguggugaccucugcccua

SEQ ID NO: 798 - RNA, 21 nt, Artificial Sequence
  Description of Artificial Sequence: Synthetic oligonucleotide
guccuggcuuucuccggcaua

SEQ ID NO: 799 - RNA, 21 nt, Artificial Sequence
  Description of Artificial Sequence: Synthetic oligonucleotide
uccggcaucaugauuguguaa

SEQ ID NO: 800 - RNA, 21 nt, Artificial Sequence
  Description of Artificial Sequence: Synthetic oligonucleotide
uaccgccggaagcaccaggaa

SEQ ID NO: 801 - RNA, 21 nt, Artificial Sequence
  Description of Artificial Sequence: Synthetic oligonucleotide
caccaggagcugcaagccaua

SEQ ID NO: 802 - RNA, 21 nt, Artificial Sequence
  Description of Artificial Sequence: Synthetic oligonucleotide
caagccaugcagauggagcua

SEQ ID NO: 803 - RNA, 21 nt, Artificial Sequence
  Description of Artificial Sequence: Synthetic oligonucleotide
auggagcugcagagcccugaa

SEQ ID NO: 804 - RNA, 21 nt, Artificial Sequence
  Description of Artificial Sequence: Synthetic oligonucleotide
ccugaguacaagcugagcaaa

SEQ ID NO: 805 - RNA, 21 nt, Artificial Sequence
  Description of Artificial Sequence: Synthetic oligonucleotide
gcuccgcaccucgaccaucaa

SEQ ID NO: 806 - RNA, 21 nt, Artificial Sequence
  Description of Artificial Sequence: Synthetic oligonucleotide
accaucaugaccgacuacaaa

SEQ ID NO: 807 - RNA, 21 nt, Artificial Sequence
  Description of Artificial Sequence: Synthetic oligonucleotide
caacuacugcuuugcuggcaa

SEQ ID NO: 808 - RNA, 21 nt, Artificial Sequence
  Description of Artificial Sequence: Synthetic oligonucleotide
ugcuggcaagaccuccuccaa

SEQ ID NO: 809 - RNA, 21 nt, Artificial Sequence
  Description of Artificial Sequence: Synthetic oligonucleotide
uccuccaucagugaccugaaa

SEQ ID NO: 810 - RNA, 21 nt, Artificial Sequence
  Description of Artificial Sequence: Synthetic oligonucleotide
ugaaggaggugccgcggaaaa

SEQ ID NO: 811 - RNA, 21 nt, Artificial Sequence
  Description of Artificial Sequence: Synthetic oligonucleotide
gagguguaugaaggccaggua

SEQ ID NO: 812 - RNA, 21 nt, Artificial Sequence
  Description of Artificial Sequence: Synthetic oligonucleotide
gguguccggaaugcccaacga

SEQ ID NO: 813 - RNA, 21 nt, Artificial Sequence
  Description of Artificial Sequence: Synthetic oligonucleotide
gcugugaagacgcugccugaa

SEQ ID NO: 814 - RNA, 21 nt, Artificial Sequence
  Description of Artificial Sequence: Synthetic oligonucleotide
ugugcucugaacaggacgaaa

SEQ ID NO: 815 - RNA, 21 nt, Artificial Sequence
  Description of Artificial Sequence: Synthetic oligonucleotide
ggacgaacuggauuuccucaa

SEQ ID NO: 816 - RNA, 21 nt, Artificial Sequence
  Description of Artificial Sequence: Synthetic oligonucleotide
uuccucauggaagcccugaua

SEQ ID NO: 817 - RNA, 21 nt, Artificial Sequence
  Description of Artificial Sequence: Synthetic oligonucleotide
gaucaucagcaaauucaacca

SEQ ID NO: 818 - RNA, 21 nt, Artificial Sequence
  Description of Artificial Sequence: Synthetic oligonucleotide
caaccaccagaacauuguuca

SEQ ID NO: 819 - RNA, 21 nt, Artificial Sequence
  Description of Artificial Sequence: Synthetic oligonucleotide
cgguucauccugcuggagcua

SEQ ID NO: 820 - RNA, 21 nt, Artificial Sequence
  Description of Artificial Sequence: Synthetic oligonucleotide
agaccucaaguccuuccucca

SEQ ID NO: 821 - RNA, 21 nt, Artificial Sequence
  Description of Artificial Sequence: Synthetic oligonucleotide
augcuggaccuucugcacgua

SEQ ID NO: 822 - RNA, 21 nt, Artificial Sequence
  Description of Artificial Sequence: Synthetic oligonucleotide
ucgggacauugccuguggcua

SEQ ID NO: 823 - RNA, 21 nt, Artificial Sequence
  Description of Artificial Sequence: Synthetic oligonucleotide
guggcugucaguauuuggaga

SEQ ID NO: 824 - RNA, 21 nt, Artificial Sequence
  Description of Artificial Sequence: Synthetic oligonucleotide
uggaggaaaaccacuucauca

SEQ ID NO: 825 - RNA, 21 nt, Artificial Sequence
  Description of Artificial Sequence: Synthetic oligonucleotide
ccgagacauugcugccagaaa

SEQ ID NO: 826 - RNA, 21 nt, Artificial Sequence
  Description of Artificial Sequence: Synthetic oligonucleotide
ugccagaaacugccucuugaa

SEQ ID NO: 827 - RNA, 21 nt, Artificial Sequence
  Description of Artificial Sequence: Synthetic oligonucleotide
ccucuugaccuguccaggcca

SEQ ID NO: 828 - RNA, 21 nt, Artificial Sequence
  Description of Artificial Sequence: Synthetic oligonucleotide
gcccuggaagaguggccaaga

SEQ ID NO: 829 - RNA, 21 nt, Artificial Sequence
  Description of Artificial Sequence: Synthetic oligonucleotide
ggccaagauuggagacuucga

SEQ ID NO: 830 - RNA, 21 nt, Artificial Sequence
  Description of Artificial Sequence: Synthetic oligonucleotide
cuucgggauggcccgagacaa

SEQ ID NO: 831 - RNA, 21 nt, Artificial Sequence
  Description of Artificial Sequence: Synthetic oligonucleotide
ccgagacaucuacagggcgaa

SEQ ID NO: 832 - RNA, 21 nt, Artificial Sequence
  Description of Artificial Sequence: Synthetic oligonucleotide
gagcuacuauagaaagggaga

SEQ ID NO: 833 - RNA, 21 nt, Artificial Sequence
  Description of Artificial Sequence: Synthetic oligonucleotide
gggaggcugugccaugcugca

SEQ ID NO: 834 - RNA, 21 nt, Artificial Sequence
  Description of Artificial Sequence: Synthetic oligonucleotide
augcugccaguuaaguggaua

SEQ ID NO: 835 - RNA, 21 nt, Artificial Sequence
  Description of Artificial Sequence: Synthetic oligonucleotide
gaggccuucauggaaggaaua

SEQ ID NO: 836 - RNA, 21 nt, Artificial Sequence
  Description of Artificial Sequence: Synthetic oligonucleotide
gaaggaauauucacuucuaaa

SEQ ID NO: 837 - RNA, 21 nt, Artificial Sequence
  Description of Artificial Sequence: Synthetic oligonucleotide
acuucuaaaacagacacauga

SEQ ID NO: 838 - RNA, 21 nt, Artificial Sequence
  Description of Artificial Sequence: Synthetic oligonucleotide
acaugguccuuuggagugcua

SEQ ID NO: 839 - RNA, 21 nt, Artificial Sequence
  Description of Artificial Sequence: Synthetic oligonucleotide
gugcugcuaugggaaaucuua

SEQ ID NO: 840 - RNA, 21 nt, Artificial Sequence
  Description of Artificial Sequence: Synthetic oligonucleotide
gaaaucuuuucucuuggauaa

SEQ ID NO: 841 - RNA, 21 nt, Artificial Sequence
  Description of Artificial Sequence: Synthetic oligonucleotide
caaaagcaaccaggaaguuca

SEQ ID NO: 842 - RNA, 21 nt, Artificial Sequence
  Description of Artificial Sequence: Synthetic oligonucleotide
ggaaguucuggaguuugucaa

SEQ ID NO: 843 - RNA, 21 nt, Artificial Sequence
  Description of Artificial Sequence: Synthetic oligonucleotide
guuugucaccaguggaggcca

SEQ ID NO: 844 - RNA, 21 nt, Artificial Sequence
  Description of Artificial Sequence: Synthetic oligonucleotide
ccacccaagaacugcccugga

SEQ ID NO: 845 - RNA, 21 nt, Artificial Sequence
  Description of Artificial Sequence: Synthetic oligonucleotide
ugcccugggccuguauaccga

SEQ ID NO: 846 - RNA, 21 nt, Artificial Sequence
  Description of Artificial Sequence: Synthetic oligonucleotide
guauaccggauaaugacucaa

SEQ ID NO: 847 - RNA, 21 nt, Artificial Sequence
  Description of Artificial Sequence: Synthetic oligonucleotide
gcuggcaacaucagccugaaa

SEQ ID NO: 848 - RNA, 21 nt, Artificial Sequence
  Description of Artificial Sequence: Synthetic oligonucleotide
agccugaagacaggcccaaca

SEQ ID NO: 849 - RNA, 21 nt, Artificial Sequence
  Description of Artificial Sequence: Synthetic oligonucleotide
aacuuugccaucauuuuggaa

SEQ ID NO: 850 - RNA, 21 nt, Artificial Sequence
  Description of Artificial Sequence: Synthetic oligonucleotide
ggagaggauugaauacugcaa

SEQ ID NO: 851 - RNA, 21 nt, Artificial Sequence
  Description of Artificial Sequence: Synthetic oligonucleotide
acccaggacccggauguaaua

SEQ ID NO: 852 - RNA, 21 nt, Artificial Sequence
  Description of Artificial Sequence: Synthetic oligonucleotide
gauguaaucaacaccgcuuua

SEQ ID NO: 853 - RNA, 21 nt, Artificial Sequence
  Description of Artificial Sequence: Synthetic oligonucleotide
accgcuuugccgauagaauaa

SEQ ID NO: 854 - RNA, 21 nt, Artificial Sequence
  Description of Artificial Sequence: Synthetic oligonucleotide
auagaauaugguccacuugua

SEQ ID NO: 855 - RNA, 21 nt, Artificial Sequence
  Description of Artificial Sequence: Synthetic oligonucleotide
ccacuuguggaagaggaagaa

SEQ ID NO: 856 - RNA, 21 nt, Artificial Sequence
  Description of Artificial Sequence: Synthetic oligonucleotide
ggaagagaaagugccugugaa

SEQ ID NO: 857 - RNA, 21 nt, Artificial Sequence
  Description of Artificial Sequence: Synthetic oligonucleotide
ugaggcccaaggacccugaga

SEQ ID NO: 858 - RNA, 21 nt, Artificial Sequence
  Description of Artificial Sequence: Synthetic oligonucleotide
ccuccucuccuggucucucaa

SEQ ID NO: 859 - RNA, 21 nt, Artificial Sequence
  Description of Artificial Sequence: Synthetic oligonucleotide
ucucucaacaggcaaaacgga

SEQ ID NO: 860 - RNA, 21 nt, Artificial Sequence
  Description of Artificial Sequence: Synthetic oligonucleotide
ucugccuaccaccuccucuga

SEQ ID NO: 861 - RNA, 21 nt, Artificial Sequence
  Description of Artificial Sequence: Synthetic oligonucleotide
cuccucuggcaaggcugcaaa

SEQ ID NO: 862 - RNA, 21 nt, Artificial Sequence
  Description of Artificial Sequence: Synthetic oligonucleotide
cugcaaagaaacccacagcua

SEQ ID NO: 863 - RNA, 21 nt, Artificial Sequence
  Description of Artificial Sequence: Synthetic oligonucleotide
gcagagaucucuguucgagua

SEQ ID NO: 864 - RNA, 21 nt, Artificial Sequence
  Description of Artificial Sequence: Synthetic oligonucleotide
guucgagucccuagagggcca

SEQ ID NO: 865 - RNA, 21 nt, Artificial Sequence
  Description of Artificial Sequence: Synthetic oligonucleotide
gugaauauggcauucucucaa

SEQ ID NO: 866 - RNA, 21 nt, Artificial Sequence
  Description of Artificial Sequence: Synthetic oligonucleotide
cucucaguccaacccuccuua

SEQ ID NO: 867 - RNA, 21 nt, Artificial Sequence
  Description of Artificial Sequence: Synthetic oligonucleotide
ccuccuucggaguugcacaaa

SEQ ID NO: 868 - RNA, 21 nt, Artificial Sequence
  Description of Artificial Sequence: Synthetic oligonucleotide
ccacggauccagaaacaagca

SEQ ID NO: 869 - RNA, 21 nt, Artificial Sequence
  Description of Artificial Sequence: Synthetic oligonucleotide
caccagcuuguggaacccaaa

SEQ ID NO: 870 - RNA, 21 nt, Artificial Sequence
  Description of Artificial Sequence: Synthetic oligonucleotide
gaacccaacguacggcuccua

SEQ ID NO: 871 - RNA, 21 nt, Artificial Sequence
  Description of Artificial Sequence: Synthetic oligonucleotide
uccugguuuacagagaaacca

SEQ ID NO: 872 - RNA, 21 nt, Artificial Sequence
  Description of Artificial Sequence: Synthetic oligonucleotide
uaauccuauagcaaagaagga

SEQ ID NO: 873 - RNA, 21 nt, Artificial Sequence
  Description of Artificial Sequence: Synthetic oligonucleotide
agaaggagccacacgacagga

SEQ ID NO: 874 - RNA, 21 nt, Artificial Sequence
  Description of Artificial Sequence: Synthetic oligonucleotide
agggaagcuguacugucccaa

SEQ ID NO: 875 - RNA, 21 nt, Artificial Sequence
  Description of Artificial Sequence: Synthetic oligonucleotide
ccaccuaacguugcaacugga

SEQ ID NO: 876 - RNA, 21 nt, Artificial Sequence
  Description of Artificial Sequence: Synthetic oligonucleotide
acugcuccuagagcccucuua

SEQ ID NO: 877 - RNA, 21 nt, Artificial Sequence
  Description of Artificial Sequence: Synthetic oligonucleotide
ucgcugacugccaauaugaaa

SEQ ID NO: 878 - RNA, 21 nt, Artificial Sequence
  Description of Artificial Sequence: Synthetic oligonucleotide
aauaugaaggagguaccucua

SEQ ID NO: 879 - RNA, 21 nt, Artificial Sequence
  Description of Artificial Sequence: Synthetic oligonucleotide
guaccucuguucaggcuacga

SEQ ID NO: 880 - RNA, 21 nt, Artificial Sequence
  Description of Artificial Sequence: Synthetic oligonucleotide
gcuacgucacuucccuuguga

SEQ ID NO: 881 - RNA, 21 nt, Artificial Sequence
  Description of Artificial Sequence: Synthetic oligonucleotide
uugugggaaugucaauuacga

SEQ ID NO: 882 - RNA, 21 nt, Artificial Sequence
  Description of Artificial Sequence: Synthetic oligonucleotide
ggcuaccagcaacagggcuua

SEQ ID NO: 883 - RNA, 21 nt, Artificial Sequence
  Description of Artificial Sequence: Synthetic oligonucleotide
uugcccuuagaagccgcuaca

SEQ ID NO: 884 - RNA, 21 nt, Artificial Sequence
  Description of Artificial Sequence: Synthetic oligonucleotide
uggagcuggucauuacgagga

SEQ ID NO: 885 - RNA, 21 nt, Artificial Sequence
  Description of Artificial Sequence: Synthetic oligonucleotide
uuacgaggauaccauucugaa

SEQ ID NO: 886 - RNA, 21 nt, Artificial Sequence
  Description of Artificial Sequence: Synthetic oligonucleotide
cauucugaaaagcaagaauaa

SEQ ID NO: 887 - RNA, 21 nt, Artificial Sequence
  Description of Artificial Sequence: Synthetic oligonucleotide
agaauagcaugaaccagccua

SEQ ID NO: 888 - RNA, 21 nt, Artificial Sequence
  Description of Artificial Sequence: Synthetic oligonucleotide
gagcucggucgcacacucaca

SEQ ID NO: 889 - RNA, 21 nt, Artificial Sequence
  Description of Artificial Sequence: Synthetic oligonucleotide
acacucacuucucuuccuuga

SEQ ID NO: 890 - RNA, 21 nt, Artificial Sequence
  Description of Artificial Sequence: Synthetic oligonucleotide
gggaucccuaagaccguggaa

SEQ ID NO: 891 - RNA, 21 nt, Artificial Sequence
  Description of Artificial Sequence: Synthetic oligonucleotide
accguggaggagagagaggca

SEQ ID NO: 892 - RNA, 21 nt, Artificial Sequence
  Description of Artificial Sequence: Synthetic oligonucleotide
agagaggcaauggcuccuuca

SEQ ID NO: 893 - RNA, 21 nt, Artificial Sequence
  Description of Artificial Sequence: Synthetic oligonucleotide
gcuccuucacaaaccagagaa

SEQ ID NO: 894 - RNA, 21 nt, Artificial Sequence
  Description of Artificial Sequence: Synthetic oligonucleotide
gagaccaaaugucacguuuua

SEQ ID NO: 895 - RNA, 21 nt, Artificial Sequence
  Description of Artificial Sequence: Synthetic oligonucleotide
cacguuuuguuuugugccaaa

SEQ ID NO: 896 - RNA, 21 nt, Artificial Sequence
  Description of Artificial Sequence: Synthetic oligonucleotide
gccaaccuauuuugaaguaca

SEQ ID NO: 897 - RNA, 21 nt, Artificial Sequence
  Description of Artificial Sequence: Synthetic oligonucleotide
uguauuuugaaaaugcuuuaa

SEQ ID NO: 898 - RNA, 21 nt, Artificial Sequence
  Description of Artificial Sequence: Synthetic oligonucleotide
uuuagaaagguuuugagcaua

SEQ ID NO: 899 - RNA, 21 nt, Artificial Sequence
  Description of Artificial Sequence: Synthetic oligonucleotide
uugagcauggguucauccuaa

SEQ ID NO: 900 - RNA, 21 nt, Artificial Sequence
  Description of Artificial Sequence: Synthetic oligonucleotide
cauccuauucuuucgaaagaa

SEQ ID NO: 901 - RNA, 21 nt, Artificial Sequence
  Description of Artificial Sequence: Synthetic oligonucleotide
aaugagugauaaauacaagga

SEQ ID NO: 902 - RNA, 21 nt, Artificial Sequence
  Description of Artificial Sequence: Synthetic oligonucleotide
auacaaggcccagauguggua

SEQ ID NO: 903 - RNA, 21 nt, Artificial Sequence
  Description of Artificial Sequence: Synthetic oligonucleotide
gaugugguugcauaagguuua

SEQ ID NO: 904 - RNA, 21 nt, Artificial Sequence
  Description of Artificial Sequence: Synthetic oligonucleotide
ugcauguuuguuguauacuua

SEQ ID NO: 905 - RNA, 21 nt, Artificial Sequence
  Description of Artificial Sequence: Synthetic oligonucleotide
uuccuuaugcuucuuucaaaa

SEQ ID NO: 906 - RNA, 21 nt, Artificial Sequence
  Description of Artificial Sequence: Synthetic oligonucleotide
uuucaaauugugugugcucua

SEQ ID NO: 907 - RNA, 21 nt, Artificial Sequence
  Description of Artificial Sequence: Synthetic oligonucleotide
cugcuucaauguagucagaaa

SEQ ID NO: 908 - RNA, 21 nt, Artificial Sequence
  Description of Artificial Sequence: Synthetic oligonucleotide
agucagaauuagcugcuucua

SEQ ID NO: 909 - RNA, 21 nt, Artificial Sequence
  Description of Artificial Sequence: Synthetic oligonucleotide
ugcuucuauguuucauaguua

SEQ ID NO: 910 - RNA, 21 nt, Artificial Sequence
  Description of Artificial Sequence: Synthetic oligonucleotide
auguuuccuugccuuguugaa

SEQ ID NO: 911 - RNA, 21 nt, Artificial Sequence
  Description of Artificial Sequence: Synthetic oligonucleotide
uguggacaugagccauuugaa

SEQ ID NO: 912 - RNA, 21 nt, Artificial Sequence
  Description of Artificial Sequence: Synthetic oligonucleotide
acggaaauaaaggaguuauua

SEQ ID NO: 913 - RNA, 21 nt, Artificial Sequence
  Description of Artificial Sequence: Synthetic oligonucleotide
aguuauuuguaaugacuaaaa

SEQ ID NO: 914 - RNA, 23 nt, Artificial Sequence
  Description of Artificial Sequence: Synthetic oligonucleotide
uagccgcuggaucgcaucugccu

SEQ ID NO: 915 - RNA, 23 nt, Artificial Sequence
  Description of Artificial Sequence: Synthetic oligonucleotide
ugguaccagcugcuaccaccgcu

SEQ ID NO: 916 - RNA, 23 nt, Artificial Sequence
  Description of Artificial Sequence: Synthetic oligonucleotide
uuccucacccaucaucagcgccc

SEQ ID NO: 917 - RNA, 23 nt, Artificial Sequence
  Description of Artificial Sequence: Synthetic oligonucleotide
ucuccucagaguucgcaggcacu

SEQ ID NO: 918 - RNA, 23 nt, Artificial Sequence
  Description of Artificial Sequence: Synthetic oligonucleotide
uugcgcaaguuugcagcguccuu

SEQ ID NO: 919 - RNA, 23 nt, Artificial Sequence
  Description of Artificial Sequence: Synthetic oligonucleotide
uuucugggcgugaaucccagccc

SEQ ID NO: 920 - RNA, 23 nt, Artificial Sequence
  Description of Artificial Sequence: Synthetic oligonucleotide
uugccugcugaacuucugggcgu

SEQ ID NO: 921 - RNA, 23 nt, Artificial Sequence
  Description of Artificial Sequence: Synthetic oligonucleotide
ugaaggcuucggacugucugccu

SEQ ID NO: 922 - RNA, 23 nt, Artificial Sequence
  Description of Artificial Sequence: Synthetic oligonucleotide
ucucaagcuaucucuccgcugcg

SEQ ID NO: 923 - RNA, 23 nt, Artificial Sequence
  Description of Artificial Sequence: Synthetic oligonucleotide
uugccgucuugcgcacccucaag

SEQ ID NO: 924 - RNA, 23 nt, Artificial Sequence
  Description of Artificial Sequence: Synthetic oligonucleotide
uuccuccaagcucuucugcccgg

SEQ ID NO: 925 - RNA, 23 nt, Artificial Sequence
  Description of Artificial Sequence: Synthetic oligonucleotide
uuuuugcguuccuuuuggcuccu

SEQ ID NO: 926 - RNA, 23 nt, Artificial Sequence
  Description of Artificial Sequence: Synthetic oligonucleotide
uacgcuguccuggccgccuuuug

SEQ ID NO: 927 - RNA, 23 nt, Artificial Sequence
  Description of Artificial Sequence: Synthetic oligonucleotide
uuuuaaggcugagaacggcggcu

SEQ ID NO: 928 - RNA, 23 nt, Artificial Sequence
  Description of Artificial Sequence: Synthetic oligonucleotide
uaaucucugcaacuuuuaaggcu

SEQ ID NO: 929 - RNA, 23 nt, Artificial Sequence
  Description of Artificial Sequence: Synthetic oligonucleotide
ugguggcaguuggguaccguccu

SEQ ID NO: 930 - RNA, 23 nt, Artificial Sequence
  Description of Artificial Sequence: Synthetic oligonucleotide
uugguugaagggagguggcaguu

SEQ ID NO: 931 - RNA, 23 nt, Artificial Sequence
  Description of Artificial Sequence: Synthetic oligonucleotide
uagaggaacuacuaugguugaag

SEQ ID NO: 932 - RNA, 23 nt, Artificial Sequence
  Description of Artificial Sequence: Synthetic oligonucleotide
uugcgcucgguacagaggaacua

SEQ ID NO: 933 - RNA, 23 nt, Artificial Sequence
  Description of Artificial Sequence: Synthetic oligonucleotide
uucuguagcucgcugcgcucggu

SEQ ID NO: 934 - RNA, 23 nt, Artificial Sequence
  Description of Artificial Sequence: Synthetic oligonucleotide
uacuggcugggaccuugagccuc

SEQ ID NO: 935 - RNA, 23 nt, Artificial Sequence
  Description of Artificial Sequence: Synthetic oligonucleotide
uagcacacugggcucacuggcug

SEQ ID NO: 936 - RNA, 23 nt, Artificial Sequence
  Description of Artificial Sequence: Synthetic oligonucleotide
ucagagacacucaagcacacugg

SEQ ID NO: 937 - RNA, 23 nt, Artificial Sequence
  Description of Artificial Sequence: Synthetic oligonucleotide
ugagucuaaaugaaacagaccug

SEQ ID NO: 938 - RNA, 23 nt, Artificial Sequence
  Description of Artificial Sequence: Synthetic oligonucleotide
uaacugcacggaggcgagcagga

SEQ ID NO: 939 - RNA, 23 nt, Artificial Sequence
  Description of Artificial Sequence: Synthetic oligonucleotide
ucgcgcaagucucuugcuuuccc

SEQ ID NO: 940 - RNA, 23 nt, Artificial Sequence
  Description of Artificial Sequence: Synthetic oligonucleotide
uagaggacugugcgugcgcgcaa

SEQ ID NO: 941 - RNA, 23 nt, Artificial Sequence
  Description of Artificial Sequence: Synthetic oligonucleotide
uuccaccugaucuccagaggacu

SEQ ID NO: 942 - RNA, 23 nt, Artificial Sequence
  Description of Artificial Sequence: Synthetic oligonucleotide
ucuugguacccagcggcuccuuc

SEQ ID NO: 943 - RNA, 23 nt, Artificial Sequence
  Description of Artificial Sequence: Synthetic oligonucleotide
ucucugaacaguccuugguaccc

SEQ ID NO: 944 - RNA, 23 nt, Artificial Sequence
  Description of Artificial Sequence: Synthetic oligonucleotide
ugagaugggaagaggcucugaac

SEQ ID NO: 945 - RNA, 23 nt, Artificial Sequence
  Description of Artificial Sequence: Synthetic oligonucleotide
uccguuuacacugcucuccgggc

SEQ ID NO: 946 - RNA, 23 nt, Artificial Sequence
  Description of Artificial Sequence: Synthetic oligonucleotide
uacaggagcccgauggcucccau

SEQ ID NO: 947 - RNA, 23 nt, Artificial Sequence
  Description of Artificial Sequence: Synthetic oligonucleotide
uagcugccguggaaagcagcagc

SEQ ID NO: 948 - RNA, 23 nt, Artificial Sequence
  Description of Artificial Sequence: Synthetic oligonucleotide
uaggcgcgaguagcugaguggcu

SEQ ID NO: 949 - RNA, 23 nt, Artificial Sequence
  Description of Artificial Sequence: Synthetic oligonucleotide
uacugccagacucuuccucugca

SEQ ID NO: 950 - RNA, 23 nt, Artificial Sequence
  Description of Artificial Sequence: Synthetic oligonucleotide
uaccacgaagucaacugccagac

SEQ ID NO: 951 - RNA, 23 nt, Artificial Sequence
  Description of Artificial Sequence: Synthetic oligonucleotide
uaagagcgagggcaccacgaagu

SEQ ID NO: 952 - RNA, 23 nt, Artificial Sequence
  Description of Artificial Sequence: Synthetic oligonucleotide
ugcguagacacggaagagcgagg

SEQ ID NO: 953 - RNA, 23 nt, Artificial Sequence
  Description of Artificial Sequence: Synthetic oligonucleotide
uaguaggucccgggcguagacac

SEQ ID NO: 954 - RNA, 23 nt, Artificial Sequence
  Description of Artificial Sequence: Synthetic oligonucleotide
ugaugguggcagcaguagguccc

SEQ ID NO: 955 - RNA, 23 nt, Artificial Sequence
  Description of Artificial Sequence: Synthetic oligonucleotide
uugccagccuucagcuccgagga

SEQ ID NO: 956 - RNA, 23 nt, Artificial Sequence
  Description of Artificial Sequence: Synthetic oligonucleotide
uccuucagcacccuggacagcgu

SEQ ID NO: 957 - RNA, 23 nt, Artificial Sequence
  Description of Artificial Sequence: Synthetic oligonucleotide
uagcaccaacugcuuggcacgcc

SEQ ID NO: 958 - RNA, 23 nt, Artificial Sequence
  Description of Artificial Sequence: Synthetic oligonucleotide
uucgcccagcuccagcaccaacu

SEQ ID NO: 959 - RNA, 23 nt, Artificial Sequence
  Description of Artificial Sequence: Synthetic oligonucleotide
ucaacccuccaagaucgccuccu

SEQ ID NO: 960 - RNA, 23 nt, Artificial Sequence
  Description of Artificial Sequence: Synthetic oligonucleotide
ucugagauugaacuggagcagcc

SEQ ID NO: 961 - RNA, 23 nt, Artificial Sequence
  Description of Artificial Sequence: Synthetic oligonucleotide
uaccaacugaacagcucgcugag

SEQ ID NO: 962 - RNA, 23 nt, Artificial Sequence
  Description of Artificial Sequence: Synthetic oligonucleotide
ucgccuuggcgaauccaccaacu

SEQ ID NO: 963 - RNA, 23 nt, Artificial Sequence
  Description of Artificial Sequence: Synthetic oligonucleotide
uaggcggauccucagucgcccuu

SEQ ID NO: 964 - RNA, 23 nt, Artificial Sequence
  Description of Artificial Sequence: Synthetic oligonucleotide
uuucucgggcaucaggcggaucc

SEQ ID NO: 965 - RNA, 23 nt, Artificial Sequence
  Description of Artificial Sequence: Synthetic oligonucleotide
uuuccgacgccuucuucucgggc

SEQ ID NO: 966 - RNA, 23 nt, Artificial Sequence
  Description of Artificial Sequence: Synthetic oligonucleotide
uacagccuucccucucugcccac

SEQ ID NO: 967 - RNA, 23 nt, Artificial Sequence
  Description of Artificial Sequence: Synthetic oligonucleotide
ugcgaauugccgcggacagccuu

SEQ ID NO: 968 - RNA, 23 nt, Artificial Sequence
  Description of Artificial Sequence: Synthetic oligonucleotide
ucgaagaucuggaagagaaggcg

SEQ ID NO: 969 - RNA, 23 nt, Artificial Sequence
  Description of Artificial Sequence: Synthetic oligonucleotide
uuccaaggagcuaugaccagucc

SEQ ID NO: 970 - RNA, 23 nt, Artificial Sequence
  Description of Artificial Sequence: Synthetic oligonucleotide
uguuuguuggugauuccaaggag

SEQ ID NO: 971 - RNA, 23 nt, Artificial Sequence
  Description of Artificial Sequence: Synthetic oligonucleotide
uagaaggagaaggcauguuuguu

SEQ ID NO: 972 - RNA, 23 nt, Artificial Sequence
  Description of Artificial Sequence: Synthetic oligonucleotide
uuguaaaauaaucaggagaagga

SEQ ID NO: 973 - RNA, 23 nt, Artificial Sequence
  Description of Artificial Sequence: Synthetic oligonucleotide
uaggugagauuccauguaaaaua

SEQ ID NO: 974 - RNA, 23 nt, Artificial Sequence
  Description of Artificial Sequence: Synthetic oligonucleotide
ucuuucauuauccaggugagauu

SEQ ID NO: 975 - RNA, 23 nt, Artificial Sequence
  Description of Artificial Sequence: Synthetic oligonucleotide
uaaagggaaggagucuuucauua

SEQ ID NO: 976 - RNA, 23 nt, Artificial Sequence
  Description of Artificial Sequence: Synthetic oligonucleotide
ucgaugagacaggaaagggaagg

SEQ ID NO: 977 - RNA, 23 nt, Artificial Sequence
  Description of Artificial Sequence: Synthetic oligonucleotide
uagaccauaucggcugcgaugag

SEQ ID NO: 978 - RNA, 23 nt, Artificial Sequence
  Description of Artificial Sequence: Synthetic oligonucleotide
uggaagucaaagcugcacuccag

SEQ ID NO: 979 - RNA, 23 nt, Artificial Sequence
  Description of Artificial Sequence: Synthetic oligonucleotide
uuuccugaggucaugcaguggag

SEQ ID NO: 980 - RNA, 23 nt, Artificial Sequence
  Description of Artificial Sequence: Synthetic oligonucleotide
uaggaccagcucugguuccugag

SEQ ID NO: 981 - RNA, 23 nt, Artificial Sequence
  Description of Artificial Sequence: Synthetic oligonucleotide
uuccagcaaguccaucugggagg

SEQ ID NO: 982 - RNA, 23 nt, Artificial Sequence
  Description of Artificial Sequence: Synthetic oligonucleotide
ugcaucuccuuagaacgcucugc

SEQ ID NO: 983 - RNA, 23 nt, Artificial Sequence
  Description of Artificial Sequence: Synthetic oligonucleotide
uagaaaggagccucugggcaucu

SEQ ID NO: 984 - RNA, 23 nt, Artificial Sequence
  Description of Artificial Sequence: Synthetic oligonucleotide
uguguugagaaggagaaaggagc

SEQ ID NO: 985 - RNA, 23 nt, Artificial Sequence
  Description of Artificial Sequence: Synthetic oligonucleotide
uuggugugcuuggagucagcuga

SEQ ID NO: 986 - RNA, 23 nt, Artificial Sequence
  Description of Artificial Sequence: Synthetic oligonucleotide
uacggacucaggauggugugcuu

SEQ ID NO: 987 - RNA, 23 nt, Artificial Sequence
  Description of Artificial Sequence: Synthetic oligonucleotide
uugcuccucauccacggacucag

SEQ ID NO: 988 - RNA, 23 nt, Artificial Sequence
  Description of Artificial Sequence: Synthetic oligonucleotide
ugugugcagugcucacugcugcu

SEQ ID NO: 989 - RNA, 23 nt, Artificial Sequence
  Description of Artificial Sequence: Synthetic oligonucleotide
uaccgagacggccagugugcagu

SEQ ID NO: 990 - RNA, 23 nt, Artificial Sequence
  Description of Artificial Sequence: Synthetic oligonucleotide
uugcaggugccugugcaccgaga

SEQ ID NO: 991 - RNA, 23 nt, Artificial Sequence
  Description of Artificial Sequence: Synthetic oligonucleotide
ugggcaauguaccuuccagaggg

SEQ ID NO: 992 - RNA, 23 nt, Artificial Sequence
  Description of Artificial Sequence: Synthetic oligonucleotide
uaggaggaucucucuugcagccu

SEQ ID NO: 993 - RNA, 23 nt, Artificial Sequence
  Description of Artificial Sequence: Synthetic oligonucleotide
uggagugggcaucaggaggaucu

SEQ ID NO: 994 - RNA, 23 nt, Artificial Sequence
  Description of Artificial Sequence: Synthetic oligonucleotide
uuguccaaccaugcuucccugga

SEQ ID NO: 995 - RNA, 23 nt, Artificial Sequence
  Description of Artificial Sequence: Synthetic oligonucleotide
uuucccuggagcacuguccaacc

SEQ ID NO: 996 - RNA, 23 nt, Artificial Sequence
  Description of Artificial Sequence: Synthetic oligonucleotide
ugacgcccgauucuucccuggag

SEQ ID NO: 997 - RNA, 23 nt, Artificial Sequence
  Description of Artificial Sequence: Synthetic oligonucleotide
uacucgaaauggguugucuggac

SEQ ID NO: 998 - RNA, 23 nt, Artificial Sequence
  Description of Artificial Sequence: Synthetic oligonucleotide
uuguauuccagggccacucgaaa

SEQ ID NO: 999 - RNA, 23 nt, Artificial Sequence
  Description of Artificial Sequence: Synthetic oligonucleotide
uuuccacuggagauguauuccag

SEQ ID NO: 1000 - RNA, 23 nt, Artificial Sequence
  Description of Artificial Sequence: Synthetic oligonucleotide
uacaagcugcgguuuccacugga

SEQ ID NO: 1001 - RNA, 23 nt, Artificial Sequence
  Description of Artificial Sequence: Synthetic oligonucleotide
uggcaaagaaguccacugcagac

SEQ ID NO: 1002 - RNA, 23 nt, Artificial Sequence
  Description of Artificial Sequence: Synthetic oligonucleotide
ucuucacugcaguucuucagggc

SEQ ID NO: 1003 - RNA, 23 nt, Artificial Sequence
  Description of Artificial Sequence: Synthetic oligonucleotide
uugcagggccaucuuggagccug

SEQ ID NO: 1004 - RNA, 23 nt, Artificial Sequence
  Description of Artificial Sequence: Synthetic oligonucleotide
ucauuccaacaagugaaggagcu

SEQ ID NO: 1005 - RNA, 23 nt, Artificial Sequence
  Description of Artificial Sequence: Synthetic oligonucleotide
uugcccaagcuggaggacugucc

SEQ ID NO: 1006 - RNA, 23 nt, Artificial Sequence
  Description of Artificial Sequence: Synthetic oligonucleotide
uaguccugguggaagucacaggc

SEQ ID NO: 1007 - RNA, 23 nt, Artificial Sequence
  Description of Artificial Sequence: Synthetic oligonucleotide
uucucccugggcacaguccuggu

SEQ ID NO: 1008 - RNA, 23 nt, Artificial Sequence
  Description of Artificial Sequence: Synthetic oligonucleotide
uuggcucucaucuucucccuggg

SEQ ID NO: 1009 - RNA, 23 nt, Artificial Sequence
  Description of Artificial Sequence: Synthetic oligonucleotide
uaguuuccggcacaucuggcucu

SEQ ID NO: 1010 - RNA, 23 nt, Artificial Sequence
  Description of Artificial Sequence: Synthetic oligonucleotide
uaaacccacaggcaguuuccggc

SEQ ID NO: 1011 - RNA, 23 nt, Artificial Sequence
  Description of Artificial Sequence: Synthetic oligonucleotide
uaagccaucuucaaaguugcagu

SEQ ID NO: 1012 - RNA, 23 nt, Artificial Sequence
  Description of Artificial Sequence: Synthetic oligonucleotide
uguccagccacagaagccaucuu

SEQ ID NO: 1013 - RNA, 23 nt, Artificial Sequence
  Description of Artificial Sequence: Synthetic oligonucleotide
uggugacagugugccuugggucc

SEQ ID NO: 1014 - RNA, 23 nt, Artificial Sequence
  Description of Artificial Sequence: Synthetic oligonucleotide
ucugaccugccauugaggagugu

SEQ ID NO: 1015 - RNA, 23 nt, Artificial Sequence
  Description of Artificial Sequence: Synthetic oligonucleotide
uaccgggcauccuuuaggguccu

SEQ ID NO: 1016 - RNA, 23 nt, Artificial Sequence
  Description of Artificial Sequence: Synthetic oligonucleotide
uuggugguccuggaaccgggcau

SEQ ID NO: 1017 - RNA, 23 nt, Artificial Sequence
  Description of Artificial Sequence: Synthetic oligonucleotide
uagagcauggucuuggugguccu

SEQ ID NO: 1018 - RNA, 23 nt, Artificial Sequence
  Description of Artificial Sequence: Synthetic oligonucleotide
uugguacugagcaauagagcaug

SEQ ID NO: 1019 - RNA, 23 nt, Artificial Sequence
  Description of Artificial Sequence: Synthetic oligonucleotide
uacuguagcacuuucagaagcgg

SEQ ID NO: 1020 - RNA, 23 nt, Artificial Sequence
  Description of Artificial Sequence: Synthetic oligonucleotide
uugcaggaaacguagcacugguc

SEQ ID NO: 1021 - RNA, 23 nt, Artificial Sequence
  Description of Artificial Sequence: Synthetic oligonucleotide
uauggagagcucuugaucggugc

SEQ ID NO: 1022 - RNA, 23 nt, Artificial Sequence
  Description of Artificial Sequence: Synthetic oligonucleotide
uuucggagcucacauggagagcu

SEQ ID NO: 1023 - RNA, 23 nt, Artificial Sequence
  Description of Artificial Sequence: Synthetic oligonucleotide
uugagccaggacauucggagcuc

SEQ ID NO: 1024 - RNA, 23 nt, Artificial Sequence
  Description of Artificial Sequence: Synthetic oligonucleotide
uaagacuccacgaaugagccagg

SEQ ID NO: 1025 - RNA, 23 nt, Artificial Sequence
  Description of Artificial Sequence: Synthetic oligonucleotide
uuagcaccaaggacacguuuccc

SEQ ID NO: 1026 - RNA, 23 nt, Artificial Sequence
  Description of Artificial Sequence: Synthetic oligonucleotide
uguuuuguucuccacuagcacca

SEQ ID NO: 1027 - RNA, 23 nt, Artificial Sequence
  Description of Artificial Sequence: Synthetic oligonucleotide
uauccugccuugcuccuucccgg

SEQ ID NO: 1028 - RNA, 23 nt, Artificial Sequence
  Description of Artificial Sequence: Synthetic oligonucleotide
uacaugccagaccauccugccuu

SEQ ID NO: 1029 - RNA, 23 nt, Artificial Sequence
  Description of Artificial Sequence: Synthetic oligonucleotide
uggcucaagccuucauaggcggc

SEQ ID NO: 1030 - RNA, 23 nt, Artificial Sequence
  Description of Artificial Sequence: Synthetic oligonucleotide
uauccacugccacaggcucaagc

SEQ ID NO: 1031 - RNA, 23 nt, Artificial Sequence
  Description of Artificial Sequence: Synthetic oligonucleotide
uagaggcaacaccauccacugcc

SEQ ID NO: 1032 - RNA, 23 nt, Artificial Sequence
  Description of Artificial Sequence: Synthetic oligonucleotide
uaaccugucagacacaucgagga

SEQ ID NO: 1033 - RNA, 23 nt, Artificial Sequence
  Description of Artificial Sequence: Synthetic oligonucleotide
uaugcgaccaucugcagccagaa

SEQ ID NO: 1034 - RNA, 23 nt, Artificial Sequence
  Description of Artificial Sequence: Synthetic oligonucleotide
uaagccacgauggcucuggaucc

SEQ ID NO: 1035 - RNA, 23 nt, Artificial Sequence
  Description of Artificial Sequence: Synthetic oligonucleotide
ugagauauugucaaaagccacga

SEQ ID NO: 1036 - RNA, 23 nt, Artificial Sequence
  Description of Artificial Sequence: Synthetic oligonucleotide
uuccaggcugauggagauauugu

SEQ ID NO: 1037 - RNA, 23 nt, Artificial Sequence
  Description of Artificial Sequence: Synthetic oligonucleotide
ugugagguagcaguccaggcuga

SEQ ID NO: 1038 - RNA, 23 nt, Artificial Sequence
  Description of Artificial Sequence: Synthetic oligonucleotide
uucuccgcuaauggugagguagc

SEQ ID NO: 1039 - RNA, 23 nt, Artificial Sequence
  Description of Artificial Sequence: Synthetic oligonucleotide
uuauucugcaggaucuuguccuc

SEQ ID NO: 1040 - RNA, 23 nt, Artificial Sequence
  Description of Artificial Sequence: Synthetic oligonucleotide
uugauuugggugcuguauucugc

SEQ ID NO: 1041 - RNA, 23 nt, Artificial Sequence
  Description of Artificial Sequence: Synthetic oligonucleotide
uaaacagguuucuugauuugggu

SEQ ID NO: 1042 - RNA, 23 nt, Artificial Sequence
  Description of Artificial Sequence: Synthetic oligonucleotide
uuuggguuucucucaaacagguu

SEQ ID NO: 1043 - RNA, 23 nt, Artificial Sequence
  Description of Artificial Sequence: Synthetic oligonucleotide
uguuucagcuccuuguuuggguu

SEQ ID NO: 1044 - RNA, 23 nt, Artificial Sequence
  Description of Artificial Sequence: Synthetic oligonucleotide
ugucugucuuggugaauuuuccc

SEQ ID NO: 1045 - RNA, 23 nt, Artificial Sequence
  Description of Artificial Sequence: Synthetic oligonucleotide
uaaugaacuguagggucaaagau

SEQ ID NO: 1046 - RNA, 23 nt, Artificial Sequence
  Description of Artificial Sequence: Synthetic oligonucleotide
uuggugaacagccaaugaacugu

SEQ ID NO: 1047 - RNA, 23 nt, Artificial Sequence
  Description of Artificial Sequence: Synthetic oligonucleotide
uuaggcguuguugcacugugccu

SEQ ID NO: 1048 - RNA, 23 nt, Artificial Sequence
  Description of Artificial Sequence: Synthetic oligonucleotide
uucagguuggaguucugguaggc

SEQ ID NO: 1049 - RNA, 23 nt, Artificial Sequence
  Description of Artificial Sequence: Synthetic oligonucleotide
uccaccuccacgcucagguugga

SEQ ID NO: 1050 - RNA, 23 nt, Artificial Sequence
  Description of Artificial Sequence: Synthetic oligonucleotide
ugcaccuuccagaucuggaugcc

SEQ ID NO: 1051 - RNA, 23 nt, Artificial Sequence
  Description of Artificial Sequence: Synthetic oligonucleotide
uugucgguggcuggcaccuucca

SEQ ID NO: 1052 - RNA, 23 nt, Artificial Sequence
  Description of Artificial Sequence: Synthetic oligonucleotide
uagaugcuguaggugucgguggc

SEQ ID NO: 1053 - RNA, 23 nt, Artificial Sequence
  Description of Artificial Sequence: Synthetic oligonucleotide
uagcagcuccguagcccgagaug

SEQ ID NO: 1054 - RNA, 23 nt, Artificial Sequence
  Description of Artificial Sequence: Synthetic oligonucleotide
uuucuucccgccuuucccgccag

SEQ ID NO: 1055 - RNA, 23 nt, Artificial Sequence
  Description of Artificial Sequence: Synthetic oligonucleotide
ucgcaucaugguguucuucccgc

SEQ ID NO: 1056 - RNA, 23 nt, Artificial Sequence
  Description of Artificial Sequence: Synthetic oligonucleotide
uacacgccgugggaccgcaucau

SEQ ID NO: 1057 - RNA, 23 nt, Artificial Sequence
  Description of Artificial Sequence: Synthetic oligonucleotide
uaugcccagcacagacacgccgu

SEQ ID NO: 1058 - RNA, 23 nt, Artificial Sequence
  Description of Artificial Sequence: Synthetic oligonucleotide
uuucuccagguugaagaugccca

SEQ ID NO: 1059 - RNA, 23 nt, Artificial Sequence
  Description of Artificial Sequence: Synthetic oligonucleotide
uauguacagcaugucauccuucu

SEQ ID NO: 1060 - RNA, 23 nt, Artificial Sequence
  Description of Artificial Sequence: Synthetic oligonucleotide
uugcccaaccaggauguacagca

SEQ ID NO: 1061 - RNA, 23 nt, Artificial Sequence
  Description of Artificial Sequence: Synthetic oligonucleotide
uuccucucccugcugcccaacca

SEQ ID NO: 1062 - RNA, 23 nt, Artificial Sequence
  Description of Artificial Sequence: Synthetic oligonucleotide
uuggauuaacugguuuguacugg

SEQ ID NO: 1063 - RNA, 23 nt, Artificial Sequence
  Description of Artificial Sequence: Synthetic oligonucleotide
uaugcagacuuucuggauuaacu

SEQ ID NO: 1064 - RNA, 23 nt, Artificial Sequence
  Description of Artificial Sequence: Synthetic oligonucleotide
uacauuguucucuccaaugcaga

SEQ ID NO: 1065 - RNA, 23 nt, Artificial Sequence
  Description of Artificial Sequence: Synthetic oligonucleotide
uauuucuucuucuaucacauugu

SEQ ID NO: 1066 - RNA, 23 nt, Artificial Sequence
  Description of Artificial Sequence: Synthetic oligonucleotide
uacgcuucuguucacacggauuu

SEQ ID NO: 1067 - RNA, 23 nt, Artificial Sequence
  Description of Artificial Sequence: Synthetic oligonucleotide
uugcccacucaugcacgcuucug

SEQ ID NO: 1068 - RNA, 23 nt, Artificial Sequence
  Description of Artificial Sequence: Synthetic oligonucleotide
uucaucuuaaauacguagguggc

SEQ ID NO: 1069 - RNA, 23 nt, Artificial Sequence
  Description of Artificial Sequence: Synthetic oligonucleotide
ugcacuccauccuucaucuuaaa

SEQ ID NO: 1070 - RNA, 23 nt, Artificial Sequence
  Description of Artificial Sequence: Synthetic oligonucleotide
uccuccggcugcaaugaucaggg

SEQ ID NO: 1071 - RNA, 23 nt, Artificial Sequence
  Description of Artificial Sequence: Synthetic oligonucleotide
uguggaacgugucugucuuggcc

SEQ ID NO: 1072 - RNA, 23 nt, Artificial Sequence
  Description of Artificial Sequence: Synthetic oligonucleotide
uaguuauucuccagucucucugg

SEQ ID NO: 1073 - RNA, 23 nt, Artificial Sequence
  Description of Artificial Sequence: Synthetic oligonucleotide
ucuagaaccgaggaguuauucuc

SEQ ID NO: 1074 - RNA, 23 nt, Artificial Sequence
  Description of Artificial Sequence: Synthetic oligonucleotide
ucggaauugccguuuagcccuag

SEQ ID NO: 1075 - RNA, 23 nt, Artificial Sequence
  Description of Artificial Sequence: Synthetic oligonucleotide
uaccaccugcggcuccggaauug

SEQ ID NO: 1076 - RNA, 23 nt, Artificial Sequence
  Description of Artificial Sequence: Synthetic oligonucleotide
uauuccagccaccuccaccaccu

SEQ ID NO: 1077 - RNA, 23 nt, Artificial Sequence
  Description of Artificial Sequence: Synthetic oligonucleotide
uaaggaaguguuaucauuccagc

SEQ ID NO: 1078 - RNA, 23 nt, Artificial Sequence
  Description of Artificial Sequence: Synthetic oligonucleotide
uccggcccagagcaaggaagugu

SEQ ID NO: 1079 - RNA, 23 nt, Artificial Sequence
  Description of Artificial Sequence: Synthetic oligonucleotide
uccugcaaagauuuuccggccca

SEQ ID NO: 1080 - RNA, 23 nt, Artificial Sequence
  Description of Artificial Sequence: Synthetic oligonucleotide
uuccgguggcacccuccugcaaa

SEQ ID NO: 1081 - RNA, 23 nt, Artificial Sequence
  Description of Artificial Sequence: Synthetic oligonucleotide
ugcaggaauguccuccgguggca

SEQ ID NO: 1082 - RNA, 23 nt, Artificial Sequence
  Description of Artificial Sequence: Synthetic oligonucleotide
ucgccuauauauccuccgccucc

SEQ ID NO: 1083 - RNA, 23 nt, Artificial Sequence
  Description of Artificial Sequence: Synthetic oligonucleotide
ugucauuguuugaggcugcauug

SEQ ID NO: 1084 - RNA, 23 nt, Artificial Sequence
  Description of Artificial Sequence: Synthetic oligonucleotide
uaguggacugaugaaggaaaccc

SEQ ID NO: 1085 - RNA, 23 nt, Artificial Sequence
  Description of Artificial Sequence: Synthetic oligonucleotide
ugguguacaggaugcccagugga

SEQ ID NO: 1086 - RNA, 23 nt, Artificial Sequence
  Description of Artificial Sequence: Synthetic oligonucleotide
uuuccaucacuuuuaaagcuggg

SEQ ID NO: 1087 - RNA, 23 nt, Artificial Sequence
  Description of Artificial Sequence: Synthetic oligonucleotide
uugcaguuuagauaaugcuuaau

SEQ ID NO: 1088 - RNA, 23 nt, Artificial Sequence
  Description of Artificial Sequence: Synthetic oligonucleotide
uucuaccucacagugacugcagu

SEQ ID NO: 1089 - RNA, 23 nt, Artificial Sequence
  Description of Artificial Sequence: Synthetic oligonucleotide
uuccaugugacauucgucuaccu

SEQ ID NO: 1090 - RNA, 23 nt, Artificial Sequence
  Description of Artificial Sequence: Synthetic oligonucleotide
uugaccuuguggcuuucaggguc

SEQ ID NO: 1091 - RNA, 23 nt, Artificial Sequence
  Description of Artificial Sequence: Synthetic oligonucleotide
uucacagaagcagaugaccuugu

SEQ ID NO: 1092 - RNA, 23 nt, Artificial Sequence
  Description of Artificial Sequence: Synthetic oligonucleotide
uaccgucccguggucacagaagc

SEQ ID NO: 1093 - RNA, 23 nt, Artificial Sequence
  Description of Artificial Sequence: Synthetic oligonucleotide
uagacgccauccucagccagcac

SEQ ID NO: 1094 - RNA, 23 nt, Artificial Sequence
  Description of Artificial Sequence: Synthetic oligonucleotide
uggugacacaaugcaggagacgc

SEQ ID NO: 1095 - RNA, 23 nt, Artificial Sequence
  Description of Artificial Sequence: Synthetic oligonucleotide
uagcgagaguggcagguguggcu

SEQ ID NO: 1096 - RNA, 23 nt, Artificial Sequence
  Description of Artificial Sequence: Synthetic oligonucleotide
uaccacagagaggaucagcgaga

SEQ ID NO: 1097 - RNA, 23 nt, Artificial Sequence
  Description of Artificial Sequence: Synthetic oligonucleotide
uagggcagaggucaccacagaga

SEQ ID NO: 1098 - RNA, 23 nt, Artificial Sequence
  Description of Artificial Sequence: Synthetic oligonucleotide
uaugccggagaaagccaggacca

SEQ ID NO: 1099 - RNA, 23 nt, Artificial Sequence
  Description of Artificial Sequence: Synthetic oligonucleotide
uuacacaaucaugaugccggaga

SEQ ID NO: 1100 - RNA, 23 nt, Artificial Sequence
  Description of Artificial Sequence: Synthetic oligonucleotide
uuccuggugcuuccggcgguaca

SEQ ID NO: 1101 - RNA, 23 nt, Artificial Sequence
  Description of Artificial Sequence: Synthetic oligonucleotide
uauggcuugcagcuccuggugcu

SEQ ID NO: 1102 - RNA, 23 nt, Artificial Sequence
  Description of Artificial Sequence: Synthetic oligonucleotide
uagcuccaucugcauggcuugca

SEQ ID NO: 1103 - RNA, 23 nt, Artificial Sequence
  Description of Artificial Sequence: Synthetic oligonucleotide
uucagggcucugcagcuccaucu

SEQ ID NO: 1104 - RNA, 23 nt, Artificial Sequence
  Description of Artificial Sequence: Synthetic oligonucleotide
uuugcucagcuuguacucagggc

SEQ ID NO: 1105 - RNA, 23 nt, Artificial Sequence
  Description of Artificial Sequence: Synthetic oligonucleotide
uugauggucgaggugcggagcuu

SEQ ID NO: 1106 - RNA, 23 nt, Artificial Sequence
  Description of Artificial Sequence: Synthetic oligonucleotide
uuuguagucggucaugauggucg

SEQ ID NO: 1107 - RNA, 23 nt, Artificial Sequence
  Description of Artificial Sequence: Synthetic oligonucleotide
uugccagcaaagcaguaguuggg

SEQ ID NO: 1108 - RNA, 23 nt, Artificial Sequence
  Description of Artificial Sequence: Synthetic oligonucleotide
uuggaggaggucuugccagcaaa

SEQ ID NO: 1109 - RNA, 23 nt, Artificial Sequence
  Description of Artificial Sequence: Synthetic oligonucleotide
uuucaggucacugauggaggagg

SEQ ID NO: 1110 - RNA, 23 nt, Artificial Sequence
  Description of Artificial Sequence: Synthetic oligonucleotide
uuuuccgcggcaccuccuucagg

SEQ ID NO: 1111 - RNA, 23 nt, Artificial Sequence
  Description of Artificial Sequence: Synthetic oligonucleotide
uaccuggccuucauacaccuccc

SEQ ID NO: 1112 - RNA, 23 nt, Artificial Sequence
  Description of Artificial Sequence: Synthetic oligonucleotide
ucguugggcauuccggacaccug

SEQ ID NO: 1113 - RNA, 23 nt, Artificial Sequence
  Description of Artificial Sequence: Synthetic oligonucleotide
uucaggcagcgucuucacagcca

SEQ ID NO: 1114 - RNA, 23 nt, Artificial Sequence
  Description of Artificial Sequence: Synthetic oligonucleotide
uuucguccuguucagagcacacu

SEQ ID NO: 1115 - RNA, 23 nt, Artificial Sequence
  Description of Artificial Sequence: Synthetic oligonucleotide
uugaggaaauccaguucguccug

SEQ ID NO: 1116 - RNA, 23 nt, Artificial Sequence
  Description of Artificial Sequence: Synthetic oligonucleotide
uaucagggcuuccaugaggaaau

SEQ ID NO: 1117 - RNA, 23 nt, Artificial Sequence
  Description of Artificial Sequence: Synthetic oligonucleotide
ugguugaauuugcugaugaucag

SEQ ID NO: 1118 - RNA, 23 nt, Artificial Sequence
  Description of Artificial Sequence: Synthetic oligonucleotide
ugaacaauguucuggugguugaa

SEQ ID NO: 1119 - RNA, 23 nt, Artificial Sequence
  Description of Artificial Sequence: Synthetic oligonucleotide
uagcuccagcaggaugaaccggg

SEQ ID NO: 1120 - RNA, 23 nt, Artificial Sequence
  Description of Artificial Sequence: Synthetic oligonucleotide
uggaggaaggacuugaggucucc

SEQ ID NO: 1121 - RNA, 23 nt, Artificial Sequence
  Description of Artificial Sequence: Synthetic oligonucleotide
uacgugcagaagguccagcaugg

SEQ ID NO: 1122 - RNA, 23 nt, Artificial Sequence
  Description of Artificial Sequence: Synthetic oligonucleotide
uagccacaggcaaugucccgagc

SEQ ID NO: 1123 - RNA, 23 nt, Artificial Sequence
  Description of Artificial Sequence: Synthetic oligonucleotide
ucuccaaauacugacagccacag

SEQ ID NO: 1124 - RNA, 23 nt, Artificial Sequence
  Description of Artificial Sequence: Synthetic oligonucleotide
ugaugaagugguuuuccuccaaa

SEQ ID NO: 1125 - RNA, 23 nt, Artificial Sequence
  Description of Artificial Sequence: Synthetic oligonucleotide
uuucuggcagcaaugucucggug

SEQ ID NO: 1126 - RNA, 23 nt, Artificial Sequence
  Description of Artificial Sequence: Synthetic oligonucleotide
uucaagaggcaguuucuggcagc

SEQ ID NO: 1127 - RNA, 23 nt, Artificial Sequence
  Description of Artificial Sequence: Synthetic oligonucleotide
uggccuggacaggucaagaggca

SEQ ID NO: 1128 - RNA, 23 nt, Artificial Sequence
  Description of Artificial Sequence: Synthetic oligonucleotide
ucuuggccacucuuccagggccu

SEQ ID NO: 1129 - RNA, 23 nt, Artificial Sequence
  Description of Artificial Sequence: Synthetic oligonucleotide
ucgaagucuccaaucuuggccac

SEQ ID NO: 1130 - RNA, 23 nt, Artificial Sequence
  Description of Artificial Sequence: Synthetic oligonucleotide
uugucucgggccaucccgaaguc

SEQ ID NO: 1131 - RNA, 23 nt, Artificial Sequence
  Description of Artificial Sequence: Synthetic oligonucleotide
uucgcccuguagaugucucgggc

SEQ ID NO: 1132 - RNA, 23 nt, Artificial Sequence
  Description of Artificial Sequence: Synthetic oligonucleotide
ucucccuuucuauaguagcucgc

SEQ ID NO: 1133 - RNA, 23 nt, Artificial Sequence
  Description of Artificial Sequence: Synthetic oligonucleotide
ugcagcauggcacagccucccuu

SEQ ID NO: 1134 - RNA, 23 nt, Artificial Sequence
  Description of Artificial Sequence: Synthetic oligonucleotide
uauccacuuaacuggcagcaugg

SEQ ID NO: 1135 - RNA, 23 nt, Artificial Sequence
  Description of Artificial Sequence: Synthetic oligonucleotide
uauuccuuccaugaaggccucug

SEQ ID NO: 1136 - RNA, 23 nt, Artificial Sequence
  Description of Artificial Sequence: Synthetic oligonucleotide
uuuagaagugaauauuccuucca

SEQ ID NO: 1137 - RNA, 23 nt, Artificial Sequence
  Description of Artificial Sequence: Synthetic oligonucleotide
ucaugugucuguuuuagaaguga

SEQ ID NO: 1138 - RNA, 23 nt, Artificial Sequence
  Description of Artificial Sequence: Synthetic oligonucleotide
uagcacuccaaaggaccaugugu

SEQ ID NO: 1139 - RNA, 23 nt, Artificial Sequence
  Description of Artificial Sequence: Synthetic oligonucleotide
uaagauuucccauagcagcacuc

SEQ ID NO: 1140 - RNA, 23 nt, Artificial Sequence
  Description of Artificial Sequence: Synthetic oligonucleotide
uuauccaagagaaaagauuuccc

SEQ ID NO: 1141 - RNA, 23 nt, Artificial Sequence
  Description of Artificial Sequence: Synthetic oligonucleotide
ugaacuuccugguugcuuuugcu

SEQ ID NO: 1142 - RNA, 23 nt, Artificial Sequence
  Description of Artificial Sequence: Synthetic oligonucleotide
uugacaaacuccagaacuuccug

SEQ ID NO: 1143 - RNA, 23 nt, Artificial Sequence
  Description of Artificial Sequence: Synthetic oligonucleotide
uggccuccacuggugacaaacuc

SEQ ID NO: 1144 - RNA, 23 nt, Artificial Sequence
  Description of Artificial Sequence: Synthetic oligonucleotide
uccagggcaguucuugggugggu

SEQ ID NO: 1145 - RNA, 23 nt, Artificial Sequence
  Description of Artificial Sequence: Synthetic oligonucleotide
ucgguauacaggcccagggcagu

SEQ ID NO: 1146 - RNA, 23 nt, Artificial Sequence
  Description of Artificial Sequence: Synthetic oligonucleotide
uugagucauuauccgguauacag

SEQ ID NO: 1147 - RNA, 23 nt, Artificial Sequence
  Description of Artificial Sequence: Synthetic oligonucleotide
uuucaggcugauguugccagcac

SEQ ID NO: 1148 - RNA, 23 nt, Artificial Sequence
  Description of Artificial Sequence: Synthetic oligonucleotide
uguugggccugucuucaggcuga

SEQ ID NO: 1149 - RNA, 23 nt, Artificial Sequence
  Description of Artificial Sequence: Synthetic oligonucleotide
uuccaaaaugauggcaaaguugg

SEQ ID NO: 1150 - RNA, 23 nt, Artificial Sequence
  Description of Artificial Sequence: Synthetic oligonucleotide
uugcaguauucaauccucuccaa

SEQ ID NO: 1151 - RNA, 23 nt, Artificial Sequence
  Description of Artificial Sequence: Synthetic oligonucleotide
uauuacauccggguccugggugc

SEQ ID NO: 1152 - RNA, 23 nt, Artificial Sequence
  Description of Artificial Sequence: Synthetic oligonucleotide
uaaagcgguguugauuacauccg

SEQ ID NO: 1153 - RNA, 23 nt, Artificial Sequence
  Description of Artificial Sequence: Synthetic oligonucleotide
uuauucuaucggcaaagcggugu

SEQ ID NO: 1154 - RNA, 23 nt, Artificial Sequence
  Description of Artificial Sequence: Synthetic oligonucleotide
uacaaguggaccauauucuaucg

SEQ ID NO: 1155 - RNA, 23 nt, Artificial Sequence
  Description of Artificial Sequence: Synthetic oligonucleotide
uucuuccucuuccacaaguggac

SEQ ID NO: 1156 - RNA, 23 nt, Artificial Sequence
  Description of Artificial Sequence: Synthetic oligonucleotide
uucacaggcacuuucucuuccuc

SEQ ID NO: 1157 - RNA, 23 nt, Artificial Sequence
  Description of Artificial Sequence: Synthetic oligonucleotide
ucucaggguccuugggccucaca

SEQ ID NO: 1158 - RNA, 23 nt, Artificial Sequence
  Description of Artificial Sequence: Synthetic oligonucleotide
uugagagaccaggagaggaggaa

SEQ ID NO: 1159 - RNA, 23 nt, Artificial Sequence
  Description of Artificial Sequence: Synthetic oligonucleotide
uccguuuugccuguugagagacc

SEQ ID NO: 1160 - RNA, 23 nt, Artificial Sequence
  Description of Artificial Sequence: Synthetic oligonucleotide
ucagaggaggugguaggcagagg

SEQ ID NO: 1161 - RNA, 23 nt, Artificial Sequence
  Description of Artificial Sequence: Synthetic oligonucleotide
uuugcagccuugccagaggaggu

SEQ ID NO: 1162 - RNA, 23 nt, Artificial Sequence
  Description of Artificial Sequence: Synthetic oligonucleotide
uagcuguggguuucuuugcagcc

SEQ ID NO: 1163 - RNA, 23 nt, Artificial Sequence
  Description of Artificial Sequence: Synthetic oligonucleotide
uacucgaacagagaucucugcag

SEQ ID NO: 1164 - RNA, 23 nt, Artificial Sequence
  Description of Artificial Sequence: Synthetic oligonucleotide
uggcccucuagggacucgaacag

SEQ ID NO: 1165 - RNA, 23 nt, Artificial Sequence
  Description of Artificial Sequence: Synthetic oligonucleotide
uugagagaaugccauauucacgu

SEQ ID NO: 1166 - RNA, 23 nt, Artificial Sequence
  Description of Artificial Sequence: Synthetic oligonucleotide
uaaggaggguuggacugagagaa

SEQ ID NO: 1167 - RNA, 23 nt, Artificial Sequence
  Description of Artificial Sequence: Synthetic oligonucleotide
uuugugcaacuccgaaggagggu

SEQ ID NO: 1168 - RNA, 23 nt, Artificial Sequence
  Description of Artificial Sequence: Synthetic oligonucleotide
ugcuuguuucuggauccguggac

SEQ ID NO: 1169 - RNA, 23 nt, Artificial Sequence
  Description of Artificial Sequence: Synthetic oligonucleotide
uuuggguuccacaagcugguggg

SEQ ID NO: 1170 - RNA, 23 nt, Artificial Sequence
  Description of Artificial Sequence: Synthetic oligonucleotide
uaggagccguacguuggguucca

SEQ ID NO: 1171 - RNA, 23 nt, Artificial Sequence
  Description of Artificial Sequence: Synthetic oligonucleotide
ugguuucucuguaaaccaggagc

SEQ ID NO: 1172 - RNA, 23 nt, Artificial Sequence
  Description of Artificial Sequence: Synthetic oligonucleotide
uccuucuuugcuauaggauuauu

SEQ ID NO: 1173 - RNA, 23 nt, Artificial Sequence
  Description of Artificial Sequence: Synthetic oligonucleotide
uccugucguguggcuccuucuuu

SEQ ID NO: 1174 - RNA, 23 nt, Artificial Sequence
  Description of Artificial Sequence: Synthetic oligonucleotide
uugggacaguacagcuucccucc

SEQ ID NO: 1175 - RNA, 23 nt, Artificial Sequence
  Description of Artificial Sequence: Synthetic oligonucleotide
uccaguugcaacguuagguggga

SEQ ID NO: 1176 - RNA, 23 nt, Artificial Sequence
  Description of Artificial Sequence: Synthetic oligonucleotide
uaagagggcucuaggagcaguga

SEQ ID NO: 1177 - RNA, 23 nt, Artificial Sequence
  Description of Artificial Sequence: Synthetic oligonucleotide
uuucauauuggcagucagcgaag

SEQ ID NO: 1178 - RNA, 23 nt, Artificial Sequence
  Description of Artificial Sequence: Synthetic oligonucleotide
uagagguaccuccuucauauugg

SEQ ID NO: 1179 - RNA, 23 nt, Artificial Sequence
  Description of Artificial Sequence: Synthetic oligonucleotide
ucguagccugaacagagguaccu

SEQ ID NO: 1180 - RNA, 23 nt, Artificial Sequence
  Description of Artificial Sequence: Synthetic oligonucleotide
ucacaagggaagugacguagccu

SEQ ID NO: 1181 - RNA, 23 nt, Artificial Sequence
  Description of Artificial Sequence: Synthetic oligonucleotide
ucguaauugacauucccacaagg

SEQ ID NO: 1182 - RNA, 23 nt, Artificial Sequence
  Description of Artificial Sequence: Synthetic oligonucleotide
uaagcccuguugcugguagccgu

SEQ ID NO: 1183 - RNA, 23 nt, Artificial Sequence
  Description of Artificial Sequence: Synthetic oligonucleotide
uguagcggcuucuaagggcaagc

SEQ ID NO: 1184 - RNA, 23 nt, Artificial Sequence
  Description of Artificial Sequence: Synthetic oligonucleotide
uccucguaaugaccagcuccagg

SEQ ID NO: 1185 - RNA, 23 nt, Artificial Sequence
  Description of Artificial Sequence: Synthetic oligonucleotide
uucagaaugguauccucguaaug

SEQ ID NO: 1186 - RNA, 23 nt, Artificial Sequence
  Description of Artificial Sequence: Synthetic oligonucleotide
uuauucuugcuuuucagaauggu

SEQ ID NO: 1187 - RNA, 23 nt, Artificial Sequence
  Description of Artificial Sequence: Synthetic oligonucleotide
uaggcugguucaugcuauucuug

SEQ ID NO: 1188 - RNA, 23 nt, Artificial Sequence
  Description of Artificial Sequence: Synthetic oligonucleotide
ugugagugugcgaccgagcucag

SEQ ID NO: 1189 - RNA, 23 nt, Artificial Sequence
  Description of Artificial Sequence: Synthetic oligonucleotide
ucaaggaagagaagugagugugc

SEQ ID NO: 1190 - RNA, 23 nt, Artificial Sequence
  Description of Artificial Sequence: Synthetic oligonucleotide
uuccacggucuuagggaucccaa

SEQ ID NO: 1191 - RNA, 23 nt, Artificial Sequence
  Description of Artificial Sequence: Synthetic oligonucleotide
ugccucucucuccuccacggucu

SEQ ID NO: 1192 - RNA, 23 nt, Artificial Sequence
  Description of Artificial Sequence: Synthetic oligonucleotide
ugaaggagccauugccucucucu

SEQ ID NO: 1193 - RNA, 23 nt, Artificial Sequence
  Description of Artificial Sequence: Synthetic oligonucleotide
uucucugguuugugaaggagcca

SEQ ID NO: 1194 - RNA, 23 nt, Artificial Sequence
  Description of Artificial Sequence: Synthetic oligonucleotide
uaaaacgugacauuuggucucug

SEQ ID NO: 1195 - RNA, 23 nt, Artificial Sequence
  Description of Artificial Sequence: Synthetic oligonucleotide
uuuggcacaaaacaaaacgugac

SEQ ID NO: 1196 - RNA, 23 nt, Artificial Sequence
  Description of Artificial Sequence: Synthetic oligonucleotide
uguacuucaaaauagguuggcac

SEQ ID NO: 1197 - RNA, 23 nt, Artificial Sequence
  Description of Artificial Sequence: Synthetic oligonucleotide
uuaaagcauuuucaaaauacagc

SEQ ID NO: 1198 - RNA, 23 nt, Artificial Sequence
  Description of Artificial Sequence: Synthetic oligonucleotide
uaugcucaaaaccuuucuaaagc

SEQ ID NO: 1199 - RNA, 23 nt, Artificial Sequence
  Description of Artificial Sequence: Synthetic oligonucleotide
uuaggaugaacccaugcucaaaa

SEQ ID NO: 1200 - RNA, 23 nt, Artificial Sequence
  Description of Artificial Sequence: Synthetic oligonucleotide
uucuuucgaaagaauaggaugaa

SEQ ID NO: 1201 - RNA, 23 nt, Artificial Sequence
  Description of Artificial Sequence: Synthetic oligonucleotide
uccuuguauuuaucacucauuuu

SEQ ID NO: 1202 - RNA, 23 nt, Artificial Sequence
  Description of Artificial Sequence: Synthetic oligonucleotide
uaccacaucugggccuuguauuu

SEQ ID NO: 1203 - RNA, 23 nt, Artificial Sequence
  Description of Artificial Sequence: Synthetic oligonucleotide
uaaaccuuaugcaaccacaucug

SEQ ID NO: 1204 - RNA, 23 nt, Artificial Sequence
  Description of Artificial Sequence: Synthetic oligonucleotide
uaaguauacaacaaacaugcaua

SEQ ID NO: 1205 - RNA, 23 nt, Artificial Sequence
  Description of Artificial Sequence: Synthetic oligonucleotide
uuuugaaagaagcauaaggaagu

SEQ ID NO: 1206 - RNA, 23 nt, Artificial Sequence
  Description of Artificial Sequence: Synthetic oligonucleotide
uagagcacacacaauuugaaaga

SEQ ID NO: 1207 - RNA, 23 nt, Artificial Sequence
  Description of Artificial Sequence: Synthetic oligonucleotide
uuucugacuacauugaagcagag

SEQ ID NO: 1208 - RNA, 23 nt, Artificial Sequence
  Description of Artificial Sequence: Synthetic oligonucleotide
uagaagcagcuaauucugacuac

SEQ ID NO: 1209 - RNA, 23 nt, Artificial Sequence
  Description of Artificial Sequence: Synthetic oligonucleotide
uaacuaugaaacauagaagcagc

SEQ ID NO: 1210 - RNA, 23 nt, Artificial Sequence
  Description of Artificial Sequence: Synthetic oligonucleotide
uucaacaaggcaaggaaacaucu

SEQ ID NO: 1211 - RNA, 23 nt, Artificial Sequence
  Description of Artificial Sequence: Synthetic oligonucleotide
uucaaauggcucauguccacauc

SEQ ID NO: 1212 - RNA, 23 nt, Artificial Sequence
  Description of Artificial Sequence: Synthetic oligonucleotide
uaauaacuccuuuauuuccguuc

SEQ ID NO: 1213 - RNA, 23 nt, Artificial Sequence
  Description of Artificial Sequence: Synthetic oligonucleotide
uuuuagucauuacaaauaacucc

SEQ ID NO: 1214 - DNA, 23 bp, Homo sapiens
aggcagatgcgatccagcggctc

SEQ ID NO: 1215 - DNA, 23 bp, Homo sapiens
agcggtggtagcagctggtacct

SEQ ID NO: 1216 - DNA, 23 bp, Homo sapiens
gggcgctgatgatgggtgaggag

SEQ ID NO: 1217 - DNA, 23 bp, Homo sapiens
agtgcctgcgaactctgaggagc

SEQ ID NO: 1218 - DNA, 23 bp, Homo sapiens
aaggacgctgcaaacttgcgcag

SEQ ID NO: 1219 - DNA, 23 bp, Homo sapiens
gggctgggattcacgcccagaag

SEQ ID NO: 1220 - DNA, 23 bp, Homo sapiens
acgcccagaagttcagcaggcag

SEQ ID NO: 1221 - DNA, 23 bp, Homo sapiens
aggcagacagtccgaagccttcc

SEQ ID NO: 1222 - DNA, 23 bp, Homo sapiens
cgcagcggagagatagcttgagg

SEQ ID NO: 1223 - DNA, 23 bp, Homo sapiens
cttgagggtgcgcaagacggcag

SEQ ID NO: 1224 - DNA, 23 bp, Homo sapiens
ccgggcagaagagcttggaggag

SEQ ID NO: 1225 - DNA, 23 bp, Homo sapiens
aggagccaaaaggaacgcaaaag

SEQ ID NO: 1226 - DNA, 23 bp, Homo sapiens
caaaaggcggccaggacagcgtg

SEQ ID NO: 1227 - DNA, 23 bp, Homo sapiens
agccgccgttctcagccttaaaa

SEQ ID NO: 1228 - DNA, 23 bp, Homo sapiens
agccttaaaagttgcagagattg

SEQ ID NO: 1229 - DNA, 23 bp, Homo sapiens
aggacggtacccaactgccacct

SEQ ID NO: 1230 - DNA, 23 bp, Homo sapiens
aactgccacctcccttcaaccat

SEQ ID NO: 1231 - DNA, 23 bp, Homo sapiens
cttcaaccatagtagttcctctg

SEQ ID NO: 1232 - DNA, 23 bp, Homo sapiens
tagttcctctgtaccgagcgcag

SEQ ID NO: 1233 - DNA, 23 bp, Homo sapiens
accgagcgcagcgagctacagac

SEQ ID NO: 1234 - DNA, 23 bp, Homo sapiens
gaggctcaaggtcccagccagtg

SEQ ID NO: 1235 - DNA, 23 bp, Homo sapiens
cagccagtgagcccagtgtgctt

SEQ ID NO: 1236 - DNA, 23 bp, Homo sapiens
ccagtgtgcttgagtgtctctgg

SEQ ID NO: 1237 - DNA, 23 bp, Homo sapiens
caggtctgtttcatttagactcc

SEQ ID NO: 1238 - DNA, 23 bp, Homo sapiens
tcctgctcgcctccgtgcagttg

SEQ ID NO: 1239 - DNA, 23 bp, Homo sapiens
gggaaagcaagagacttgcgcgc

SEQ ID NO: 1240 - DNA, 23 bp, Homo sapiens
ttgcgcgcacgcacagtcctctg

SEQ ID NO: 1241 - DNA, 23 bp, Homo sapiens
agtcctctggagatcaggtggaa

SEQ ID NO: 1242 - DNA, 23 bp, Homo sapiens
gaaggagccgctgggtaccaagg

SEQ ID NO: 1243 - DNA, 23 bp, Homo sapiens
gggtaccaaggactgttcagagc

SEQ ID NO: 1244 - DNA, 23 bp, Homo sapiens
gttcagagcctcttcccatctcg

SEQ ID NO: 1245 - DNA, 23 bp, Homo sapiens
gcccggagagcagtgtaaacggc

SEQ ID NO: 1246 - DNA, 23 bp, Homo sapiens
atgggagccatcgggctcctgtg

SEQ ID NO: 1247 - DNA, 23 bp, Homo sapiens
gctgctgctttccacggcagctg

SEQ ID NO: 1248 - DNA, 23 bp, Homo sapiens
agccactcagctactcgcgcctg

SEQ ID NO: 1249 - DNA, 23 bp, Homo sapiens
tgcagaggaagagtctggcagtt

SEQ ID NO: 1250 - DNA, 23 bp, Homo sapiens
gtctggcagttgacttcgtggtg

SEQ ID NO: 1251 - DNA, 23 bp, Homo sapiens
acttcgtggtgccctcgctcttc

SEQ ID NO: 1252 - DNA, 23 bp, Homo sapiens
cctcgctcttccgtgtctacgcc

SEQ ID NO: 1253 - DNA, 23 bp, Homo sapiens
gtgtctacgcccgggacctactg

SEQ ID NO: 1254 - DNA, 23 bp, Homo sapiens
gggacctactgctgccaccatcc

SEQ ID NO: 1255 - DNA, 23 bp, Homo sapiens
tcctcggagctgaaggctggcag

SEQ ID NO: 1256 - DNA, 23 bp, Homo sapiens
acgctgtccagggtgctgaaggg

SEQ ID NO: 1257 - DNA, 23 bp, Homo sapiens
ggcgtgccaagcagttggtgctg

SEQ ID NO: 1258 - DNA, 23 bp, Homo sapiens
agttggtgctggagctgggcgag

SEQ ID NO: 1259 - DNA, 23 bp, Homo sapiens
aggaggcgatcttggagggttgc

SEQ ID NO: 1260 - DNA, 23 bp, Homo sapiens
ggctgctccagttcaatctcagc

SEQ ID NO: 1261 - DNA, 23 bp, Homo sapiens
ctcagcgagctgttcagttggtg

SEQ ID NO: 1262 - DNA, 23 bp, Homo sapiens
agttggtggattcgccaaggcga

SEQ ID NO: 1263 - DNA, 23 bp, Homo sapiens
aagggcgactgaggatccgcctg

SEQ ID NO: 1264 - DNA, 23 bp, Homo sapiens
ggatccgcctgatgcccgagaag

SEQ ID NO: 1265 - DNA, 23 bp, Homo sapiens
gcccgagaagaaggcgtcggaag

SEQ ID NO: 1266 - DNA, 23 bp, Homo sapiens
gtgggcagagagggaaggctgtc

SEQ ID NO: 1267 - DNA, 23 bp, Homo sapiens
aaggctgtccgcggcaattcgcg

SEQ ID NO: 1268 - DNA, 23 bp, Homo sapiens
cgccttctcttccagatcttcgg

SEQ ID NO: 1269 - DNA, 23 bp, Homo sapiens
ggactggtcatagctccttggaa

SEQ ID NO: 1270 - DNA, 23 bp, Homo sapiens
ctccttggaatcaccaacaaaca

SEQ ID NO: 1271 - DNA, 23 bp, Homo sapiens
aacaaacatgccttctccttctc

SEQ ID NO: 1272 - DNA, 23 bp, Homo sapiens
tccttctcctgattattttacat

SEQ ID NO: 1273 - DNA, 23 bp, Homo sapiens
tattttacatggaatctcacctg

SEQ ID NO: 1274 - DNA, 23 bp, Homo sapiens
aatctcacctggataatgaaaga

SEQ ID NO: 1275 - DNA, 23 bp, Homo sapiens
taatgaaagactccttccctttc

SEQ ID NO: 1276 - DNA, 23 bp, Homo sapiens
ccttccctttcctgtctcatcgc

SEQ ID NO: 1277 - DNA, 23 bp, Homo sapiens
ctcatcgcagccgatatggtctg

SEQ ID NO: 1278 - DNA, 23 bp, Homo sapiens
ctggagtgcagctttgacttccc

SEQ ID NO: 1279 - DNA, 23 bp, Homo sapiens
ctccactgcatgacctcaggaac

SEQ ID NO: 1280 - DNA, 23 bp, Homo sapiens
ctcaggaaccagagctggtcctg

SEQ ID NO: 1281 - DNA, 23 bp, Homo sapiens
cctcccagatggacttgctggat

SEQ ID NO: 1282 - DNA, 23 bp, Homo sapiens
gcagagcgttctaaggagatgcc

SEQ ID NO: 1283 - DNA, 23 bp, Homo sapiens
agatgcccagaggctcctttctc

SEQ ID NO: 1284 - DNA, 23 bp, Homo sapiens
gctcctttctccttctcaacacc

SEQ ID NO: 1285 - DNA, 23 bp, Homo sapiens
tcagctgactccaagcacaccat

SEQ ID NO: 1286 - DNA, 23 bp, Homo sapiens
aagcacaccatcctgagtccgtg

SEQ ID NO: 1287 - DNA, 23 bp, Homo sapiens
ctgagtccgtggatgaggagcag

SEQ ID NO: 1288 - DNA, 23 bp, Homo sapiens
agcagcagtgagcactgcacact

SEQ ID NO: 1289 - DNA, 23 bp, Homo sapiens
actgcacactggccgtctcggtg

SEQ ID NO: 1290 - DNA, 23 bp, Homo sapiens
tctcggtgcacaggcacctgcag

SEQ ID NO: 1291 - DNA, 23 bp, Homo sapiens
ccctctggaaggtacattgccca

SEQ ID NO: 1292 - DNA, 23 bp, Homo sapiens
aggctgcaagagagatcctcctg

SEQ ID NO: 1293 - DNA, 23 bp, Homo sapiens
agatcctcctgatgcccactcca

SEQ ID NO: 1294 - DNA, 23 bp, Homo sapiens
tccagggaagcatggttggacag

SEQ ID NO: 1295 - DNA, 23 bp, Homo sapiens
ggttggacagtgctccagggaag

SEQ ID NO: 1296 - DNA, 23 bp, Homo sapiens
ctccagggaagaatcgggcgtcc

SEQ ID NO: 1297 - DNA, 23 bp, Homo sapiens
gtccagacaacccatttcgagtg

SEQ ID NO: 1298 - DNA, 23 bp, Homo sapiens
tttcgagtggccctggaatacat

SEQ ID NO: 1299 - DNA, 23 bp, Homo sapiens
ctggaatacatctccagtggaaa

SEQ ID NO: 1300 - DNA, 23 bp, Homo sapiens
tccagtggaaaccgcagcttgtc

SEQ ID NO: 1301 - DNA, 23 bp, Homo sapiens
gtctgcagtggacttctttgccc

SEQ ID NO: 1302 - DNA, 23 bp, Homo sapiens
gccctgaagaactgcagtgaagg

SEQ ID NO: 1303 - DNA, 23 bp, Homo sapiens
caggctccaagatggccctgcag

SEQ ID NO: 1304 - DNA, 23 bp, Homo sapiens
agctccttcacttgttggaatgg

SEQ ID NO: 1305 - DNA, 23 bp, Homo sapiens
ggacagtcctccagcttgggcag

SEQ ID NO: 1306 - DNA, 23 bp, Homo sapiens
gcctgtgacttccaccaggactg

SEQ ID NO: 1307 - DNA, 23 bp, Homo sapiens
accaggactgtgcccagggagaa

SEQ ID NO: 1308 - DNA, 23 bp, Homo sapiens
cccagggagaagatgagagccag

SEQ ID NO: 1309 - DNA, 23 bp, Homo sapiens
agagccagatgtgccggaaactg

SEQ ID NO: 1310 - DNA, 23 bp, Homo sapiens
gccggaaactgcctgtgggtttt

SEQ ID NO: 1311 - DNA, 23 bp, Homo sapiens
actgcaactttgaagatggcttc

SEQ ID NO: 1312 - DNA, 23 bp, Homo sapiens
aagatggcttctgtggctggacc

SEQ ID NO: 1313 - DNA, 23 bp, Homo sapiens
ggacccaaggcacactgtcaccc

SEQ ID NO: 1314 - DNA, 23 bp, Homo sapiens
acactcctcaatggcaggtcagg

SEQ ID NO: 1315 - DNA, 23 bp, Homo sapiens
aggaccctaaaggatgcccggtt

SEQ ID NO: 1316 - DNA, 23 bp, Homo sapiens
atgcccggttccaggaccaccaa

SEQ ID NO: 1317 - DNA, 23 bp, Homo sapiens
aggaccaccaagaccatgctcta

SEQ ID NO: 1318 - DNA, 23 bp, Homo sapiens
catgctctattgctcagtaccac

SEQ ID NO: 1319 - DNA, 23 bp, Homo sapiens
ccgcttctgaaagtgctacagtg

SEQ ID NO: 1320 - DNA, 23 bp, Homo sapiens
gaccagtgctacgtttcctgcac

SEQ ID NO: 1321 - DNA, 23 bp, Homo sapiens
gcaccgatcaagagctctccatg

SEQ ID NO: 1322 - DNA, 23 bp, Homo sapiens
agctctccatgtgagctccgaat

SEQ ID NO: 1323 - DNA, 23 bp, Homo sapiens
gagctccgaatgtcctggctcat

SEQ ID NO: 1324 - DNA, 23 bp, Homo sapiens
cctggctcattcgtggagtcttg

SEQ ID NO: 1325 - DNA, 23 bp, Homo sapiens
gggaaacgtgtccttggtgctag

SEQ ID NO: 1326 - DNA, 23 bp, Homo sapiens
tggtgctagtggagaacaaaacc

SEQ ID NO: 1327 - DNA, 23 bp, Homo sapiens
ccgggaaggagcaaggcaggatg

SEQ ID NO: 1328 - DNA, 23 bp, Homo sapiens
aaggcaggatggtctggcatgtc

SEQ ID NO: 1329 - DNA, 23 bp, Homo sapiens
gccgcctatgaaggcttgagcct

SEQ ID NO: 1330 - DNA, 23 bp, Homo sapiens
gcttgagcctgtggcagtggatg

SEQ ID NO: 1331 - DNA, 23 bp, Homo sapiens
ggcagtggatggtgttgcctctc

SEQ ID NO: 1332 - DNA, 23 bp, Homo sapiens
tcctcgatgtgtctgacaggttc

SEQ ID NO: 1333 - DNA, 23 bp, Homo sapiens
ttctggctgcagatggtcgcatg

SEQ ID NO: 1334 - DNA, 23 bp, Homo sapiens
ggatccagagccatcgtggcttt

SEQ ID NO: 1335 - DNA, 23 bp, Homo sapiens
tcgtggcttttgacaatatctcc

SEQ ID NO: 1336 - DNA, 23 bp, Homo sapiens
acaatatctccatcagcctggac

SEQ ID NO: 1337 - DNA, 23 bp, Homo sapiens
tcagcctggactgctacctcacc

SEQ ID NO: 1338 - DNA, 23 bp, Homo sapiens
gctacctcaccattagcggagag

SEQ ID NO: 1339 - DNA, 23 bp, Homo sapiens
gaggacaagatcctgcagaatac

SEQ ID NO: 1340 - DNA, 23 bp, Homo sapiens
gcagaatacagcacccaaatcaa

SEQ ID NO: 1341 - DNA, 23 bp, Homo sapiens
acccaaatcaagaaacctgtttg

SEQ ID NO: 1342 - DNA, 23 bp, Homo sapiens
aacctgtttgagagaaacccaaa

SEQ ID NO: 1343 - DNA, 23 bp, Homo sapiens
aacccaaacaaggagctgaaacc

SEQ ID NO: 1344 - DNA, 23 bp, Homo sapiens
gggaaaattcaccaagacagacc

SEQ ID NO: 1345 - DNA, 23 bp, Homo sapiens
atctttgaccctacagttcattg

SEQ ID NO: 1346 - DNA, 23 bp, Homo sapiens
acagttcattggctgttcaccac

SEQ ID NO: 1347 - DNA, 23 bp, Homo sapiens
aggcacagtgcaacaacgcctac

SEQ ID NO: 1348 - DNA, 23 bp, Homo sapiens
gcctaccagaactccaacctgag

SEQ ID NO: 1349 - DNA, 23 bp, Homo sapiens
tccaacctgagcgtggaggtggg

SEQ ID NO: 1350 - DNA, 23 bp, Homo sapiens
ggcatccagatctggaaggtgcc

SEQ ID NO: 1351 - DNA, 23 bp, Homo sapiens
tggaaggtgccagccaccgacac

SEQ ID NO: 1352 - DNA, 23 bp, Homo sapiens
gccaccgacacctacagcatctc

SEQ ID NO: 1353 - DNA, 23 bp, Homo sapiens
catctcgggctacggagctgctg

SEQ ID NO: 1354 - DNA, 23 bp, Homo sapiens
ctggcgggaaaggcgggaagaac

SEQ ID NO: 1355 - DNA, 23 bp, Homo sapiens
gcgggaagaacaccatgatgcgg

SEQ ID NO: 1356 - DNA, 23 bp, Homo sapiens
atgatgcggtcccacggcgtgtc

SEQ ID NO: 1357 - DNA, 23 bp, Homo sapiens
acggcgtgtctgtgctgggcatc

SEQ ID NO: 1358 - DNA, 23 bp, Homo sapiens
tgggcatcttcaacctggagaag

SEQ ID NO: 1359 - DNA, 23 bp, Homo sapiens
agaaggatgacatgctgtacatc

SEQ ID NO: 1360 - DNA, 23 bp, Homo sapiens
tgctgtacatcctggttgggcag

SEQ ID NO: 1361 - DNA, 23 bp, Homo sapiens
tggttgggcagcagggagaggac

SEQ ID NO: 1362 - DNA, 23 bp, Homo sapiens
ccagtacaaaccagttaatccag

SEQ ID NO: 1363 - DNA, 23 bp, Homo sapiens
agttaatccagaaagtctgcatt

SEQ ID NO: 1364 - DNA, 23 bp, Homo sapiens
tctgcattggagagaacaatgtg

SEQ ID NO: 1365 - DNA, 23 bp, Homo sapiens
acaatgtgatagaagaagaaatc

SEQ ID NO: 1366 - DNA, 23 bp, Homo sapiens
aaatccgtgtgaacagaagcgtg

SEQ ID NO: 1367 - DNA, 23 bp, Homo sapiens
cagaagcgtgcatgagtgggcag

SEQ ID NO: 1368 - DNA, 23 bp, Homo sapiens
gccacctacgtatttaagatgaa

SEQ ID NO: 1369 - DNA, 23 bp, Homo sapiens
tttaagatgaaggatggagtgcc

SEQ ID NO: 1370 - DNA, 23 bp, Homo sapiens
ccctgatcattgcagccggaggt

SEQ ID NO: 1371 - DNA, 23 bp, Homo sapiens
ggccaagacagacacgttccacc

SEQ ID NO: 1372 - DNA, 23 bp, Homo sapiens
ccagagagactggagaataactc

SEQ ID NO: 1373 - DNA, 23 bp, Homo sapiens
gagaataactcctcggttctagg

SEQ ID NO: 1374 - DNA, 23 bp, Homo sapiens
ctagggctaaacggcaattccgg

SEQ ID NO: 1375 - DNA, 23 bp, Homo sapiens
caattccggagccgcaggtggtg

SEQ ID NO: 1376 - DNA, 23 bp, Homo sapiens
aggtggtggaggtggctggaatg

SEQ ID NO: 1377 - DNA, 23 bp, Homo sapiens
gctggaatgataacacttccttg

SEQ ID NO: 1378 - DNA, 23 bp, Homo sapiens
acacttccttgctctgggccgga

SEQ ID NO: 1379 - DNA, 23 bp, Homo sapiens
tgggccggaaaatctttgcagga

SEQ ID NO: 1380 - DNA, 23 bp, Homo sapiens
tttgcaggagggtgccaccggag

SEQ ID NO: 1381 - DNA, 23 bp, Homo sapiens
tgccaccggaggacattcctgcc

SEQ ID NO: 1382 - DNA, 23 bp, Homo sapiens
ggaggcggaggatatataggcgg

SEQ ID NO: 1383 - DNA, 23 bp, Homo sapiens
caatgcagcctcaaacaatgacc

SEQ ID NO: 1384 - DNA, 23 bp, Homo sapiens
gggtttccttcatcagtccactg

SEQ ID NO: 1385 - DNA, 23 bp, Homo sapiens
tccactgggcatcctgtacaccc

SEQ ID NO: 1386 - DNA, 23 bp, Homo sapiens
cccagctttaaaagtgatggaag

SEQ ID NO: 1387 - DNA, 23 bp, Homo sapiens
attaagcattatctaaactgcag

SEQ ID NO: 1388 - DNA, 23 bp, Homo sapiens
actgcagtcactgtgaggtagac

SEQ ID NO: 1389 - DNA, 23 bp, Homo sapiens
aggtagacgaatgtcacatggac

SEQ ID NO: 1390 - DNA, 23 bp, Homo sapiens
gaccctgaaagccacaaggtcat

SEQ ID NO: 1391 - DNA, 23 bp, Homo sapiens
acaaggtcatctgcttctgtgac

SEQ ID NO: 1392 - DNA, 23 bp, Homo sapiens
gcttctgtgaccacgggacggtg

SEQ ID NO: 1393 - DNA, 23 bp, Homo sapiens
gtgctggctgaggatggcgtctc

SEQ ID NO: 1394 - DNA, 23 bp, Homo sapiens
gcgtctcctgcattgtgtcaccc

SEQ ID NO: 1395 - DNA, 23 bp, Homo sapiens
agccacacctgccactctcgctg

SEQ ID NO: 1396 - DNA, 23 bp, Homo sapiens
tctcgctgatcctctctgtggtg

SEQ ID NO: 1397 - DNA, 23 bp, Homo sapiens
tctctgtggtgacctctgccctc

SEQ ID NO: 1398 - DNA, 23 bp, Homo sapiens
tggtcctggctttctccggcatc

SEQ ID NO: 1399 - DNA, 23 bp, Homo sapiens
tctccggcatcatgattgtgtac

SEQ ID NO: 1400 - DNA, 23 bp, Homo sapiens
tgtaccgccggaagcaccaggag

SEQ ID NO: 1401 - DNA, 23 bp, Homo sapiens
agcaccaggagctgcaagccatg

SEQ ID NO: 1402 - DNA, 23 bp, Homo sapiens
tgcaagccatgcagatggagctg

SEQ ID NO: 1403 - DNA, 23 bp, Homo sapiens
agatggagctgcagagccctgag

SEQ ID NO: 1404 - DNA, 23 bp, Homo sapiens
gccctgagtacaagctgagcaag

SEQ ID NO: 1405 - DNA, 23 bp, Homo sapiens
aagctccgcacctcgaccatcat

SEQ ID NO: 1406 - DNA, 23 bp, Homo sapiens
cgaccatcatgaccgactacaac

SEQ ID NO: 1407 - DNA, 23 bp, Homo sapiens
cccaactactgctttgctggcaa

SEQ ID NO: 1408 - DNA, 23 bp, Homo sapiens
tttgctggcaagacctcctccat

SEQ ID NO: 1409 - DNA, 23 bp, Homo sapiens
cctcctccatcagtgacctgaag

SEQ ID NO: 1410 - DNA, 23 bp, Homo sapiens
cctgaaggaggtgccgcggaaaa

SEQ ID NO: 1411 - DNA, 23 bp, Homo sapiens
gggaggtgtatgaaggccaggtg

SEQ ID NO: 1412 - DNA, 23 bp, Homo sapiens
caggtgtccggaatgcccaacga

SEQ ID NO: 1413 - DNA, 23 bp, Homo sapiens
tggctgtgaagacgctgcctgaa

SEQ ID NO: 1414 - DNA, 23 bp, Homo sapiens
agtgtgctctgaacaggacgaac

SEQ ID NO: 1415 - DNA, 23 bp, Homo sapiens
caggacgaactggatttcctcat

SEQ ID NO: 1416 - DNA, 23 bp, Homo sapiens
atttcctcatggaagccctgatc

SEQ ID NO: 1417 - DNA, 23 bp, Homo sapiens
ctgatcatcagcaaattcaacca

SEQ ID NO: 1418 - DNA, 23 bp, Homo sapiens
ttcaaccaccagaacattgttcg

SEQ ID NO: 1419 - DNA, 23 bp, Homo sapiens
cccggttcatcctgctggagctc

SEQ ID NO: 1420 - DNA, 23 bp, Homo sapiens
ggagacctcaagtccttcctccg

SEQ ID NO: 1421 - DNA, 23 bp, Homo sapiens
ccatgctggaccttctgcacgtg

SEQ ID NO: 1422 - DNA, 23 bp, Homo sapiens
gctcgggacattgcctgtggctg

SEQ ID NO: 1423 - DNA, 23 bp, Homo sapiens
ctgtggctgtcagtatttggagg

SEQ ID NO: 1424 - DNA, 23 bp, Homo sapiens
tttggaggaaaaccacttcatcc

SEQ ID NO: 1425 - DNA, 23 bp, Homo sapiens
caccgagacattgctgccagaaa

SEQ ID NO: 1426 - DNA, 23 bp, Homo sapiens
gctgccagaaactgcctcttgac

SEQ ID NO: 1427 - DNA, 23 bp, Homo sapiens
tgcctcttgacctgtccaggccc

SEQ ID NO: 1428 - DNA, 23 bp, Homo sapiens
aggccctggaagagtggccaaga

SEQ ID NO: 1429 - DNA, 23 bp, Homo sapiens
gtggccaagattggagacttcgg

SEQ ID NO: 1430 - DNA, 23 bp, Homo sapiens
gacttcgggatggcccgagacat

SEQ ID NO: 1431 - DNA, 23 bp, Homo sapiens
gcccgagacatctacagggcgag

SEQ ID NO: 1432 - DNA, 23 bp, Homo sapiens
gcgagctactatagaaagggagg

SEQ ID NO: 1433 - DNA, 23 bp, Homo sapiens
aagggaggctgtgccatgctgcc

SEQ ID NO: 1434 - DNA, 23 bp, Homo sapiens
ccatgctgccagttaagtggatg

SEQ ID NO: 1435 - DNA, 23 bp, Homo sapiens
cagaggccttcatggaaggaata

SEQ ID NO: 1436 - DNA, 23 bp, Homo sapiens
tggaaggaatattcacttctaaa

SEQ ID NO: 1437 - DNA, 23 bp, Homo sapiens
tcacttctaaaacagacacatgg

SEQ ID NO: 1438 - DNA, 23 bp, Homo sapiens
acacatggtcctttggagtgctg

SEQ ID NO: 1439 - DNA, 23 bp, Homo sapiens
gagtgctgctatgggaaatcttt

SEQ ID NO: 1440 - DNA, 23 bp, Homo sapiens
gggaaatcttttctcttggatat

SEQ ID NO: 1441 - DNA, 23 bp, Homo sapiens
agcaaaagcaaccaggaagttct

SEQ ID NO: 1442 - DNA, 23 bp, Homo sapiens
caggaagttctggagtttgtcac

SEQ ID NO: 1443 - DNA, 23 bp, Homo sapiens
gagtttgtcaccagtggaggccg

SEQ ID NO: 1444 - DNA, 23 bp, Homo sapiens
acccacccaagaactgccctggg

SEQ ID NO: 1445 - DNA, 23 bp, Homo sapiens
actgccctgggcctgtataccgg

SEQ ID NO: 1446 - DNA, 23 bp, Homo sapiens
ctgtataccggataatgactcag

SEQ ID NO: 1447 - DNA, 23 bp, Homo sapiens
gtgctggcaacatcagcctgaag

SEQ ID NO: 1448 - DNA, 23 bp, Homo sapiens
tcagcctgaagacaggcccaact

SEQ ID NO: 1449 - DNA, 23 bp, Homo sapiens
ccaactttgccatcattttggag

SEQ ID NO: 1450 - DNA, 23 bp, Homo sapiens
ttggagaggattgaatactgcac

SEQ ID NO: 1451 - DNA, 23 bp, Homo sapiens
gcacccaggacccggatgtaatc

SEQ ID NO: 1452 - DNA, 23 bp, Homo sapiens
cggatgtaatcaacaccgctttg

SEQ ID NO: 1453 - DNA, 23 bp, Homo sapiens
acaccgctttgccgatagaatat

SEQ ID NO: 1454 - DNA, 23 bp, Homo sapiens
cgatagaatatggtccacttgtg

SEQ ID NO: 1455 - DNA, 23 bp, Homo sapiens
gtccacttgtggaagaggaagag

SEQ ID NO: 1456 - DNA, 23 bp, Homo sapiens
gaggaagagaaagtgcctgtgag

SEQ ID NO: 1457 - DNA, 23 bp, Homo sapiens
tgtgaggcccaaggaccctgagg

SEQ ID NO: 1458 - DNA, 23 bp, Homo sapiens
ttcctcctctcctggtctctcaa

SEQ ID NO: 1459 - DNA, 23 bp, Homo sapiens
ggtctctcaacaggcaaaacggg

SEQ ID NO: 1460 - DNA, 23 bp, Homo sapiens
cctctgcctaccacctcctctgg

SEQ ID NO: 1461 - DNA, 23 bp, Homo sapiens
acctcctctggcaaggctgcaaa

SEQ ID NO: 1462 - DNA, 23 bp, Homo sapiens
ggctgcaaagaaacccacagctg

SEQ ID NO: 1463 - DNA, 23 bp, Homo sapiens
ctgcagagatctctgttcgagtc

SEQ ID NO: 1464 - DNA, 23 bp, Homo sapiens
ctgttcgagtccctagagggccg

SEQ ID NO: 1465 - DNA, 23 bp, Homo sapiens
acgtgaatatggcattctctcag

SEQ ID NO: 1466 - DNA, 23 bp, Homo sapiens
ttctctcagtccaaccctccttc

SEQ ID NO: 1467 - DNA, 23 bp, Homo sapiens
accctccttcggagttgcacaag

SEQ ID NO: 1468 - DNA, 23 bp, Homo sapiens
gtccacggatccagaaacaagcc

SEQ ID NO: 1469 - DNA, 23 bp, Homo sapiens
cccaccagcttgtggaacccaac

SEQ ID NO: 1470 - DNA, 23 bp, Homo sapiens
tggaacccaacgtacggctcctg

SEQ ID NO: 1471 - DNA, 23 bp, Homo sapiens
gctcctggtttacagagaaaccc

SEQ ID NO: 1472 - DNA, 23 bp, Homo sapiens
aataatcctatagcaaagaagga

SEQ ID NO: 1473 - DNA, 23 bp, Homo sapiens
aaagaaggagccacacgacaggg

SEQ ID NO: 1474 - DNA, 23 bp, Homo sapiens
ggagggaagctgtactgtcccac

SEQ ID NO: 1475 - DNA, 23 bp, Homo sapiens
tcccacctaacgttgcaactggg

SEQ ID NO: 1476 - DNA, 23 bp, Homo sapiens
tcactgctcctagagccctcttc

SEQ ID NO: 1477 - DNA, 23 bp, Homo sapiens
cttcgctgactgccaatatgaag

SEQ ID NO: 1478 - DNA, 23 bp, Homo sapiens
ccaatatgaaggaggtacctctg

SEQ ID NO: 1479 - DNA, 23 bp, Homo sapiens
aggtacctctgttcaggctacgt

SEQ ID NO: 1480 - DNA, 23 bp, Homo sapiens
aggctacgtcacttcccttgtgg

SEQ ID NO: 1481 - DNA, 23 bp, Homo sapiens
ccttgtgggaatgtcaattacgg

SEQ ID NO: 1482 - DNA, 23 bp, Homo sapiens
acggctaccagcaacagggcttg

SEQ ID NO: 1483 - DNA, 23 bp, Homo sapiens
gcttgcccttagaagccgctact

SEQ ID NO: 1484 - DNA, 23 bp, Homo sapiens
cctggagctggtcattacgagga

SEQ ID NO: 1485 - DNA, 23 bp, Homo sapiens
cattacgaggataccattctgaa

SEQ ID NO: 1486 - DNA, 23 bp, Homo sapiens
accattctgaaaagcaagaatag

SEQ ID NO: 1487 - DNA, 23 bp, Homo sapiens
caagaatagcatgaaccagcctg

SEQ ID NO: 1488 - DNA, 23 bp, Homo sapiens
ctgagctcggtcgcacactcact

SEQ ID NO: 1489 - DNA, 23 bp, Homo sapiens
gcacactcacttctcttccttgg

SEQ ID NO: 1490 - DNA, 23 bp, Homo sapiens
ttgggatccctaagaccgtggag

SEQ ID NO: 1491 - DNA, 23 bp, Homo sapiens
agaccgtggaggagagagaggca

SEQ ID NO: 1492 - DNA, 23 bp, Homo sapiens
agagagaggcaatggctccttca

SEQ ID NO: 1493 - DNA, 23 bp, Homo sapiens
tggctccttcacaaaccagagac

SEQ ID NO: 1494 - DNA, 23 bp, Homo sapiens
cagagaccaaatgtcacgttttg

SEQ ID NO: 1495 - DNA, 23 bp, Homo sapiens
gtcacgttttgttttgtgccaac

SEQ ID NO: 1496 - DNA, 23 bp, Homo sapiens
gtgccaacctattttgaagtacc

SEQ ID NO: 1497 - DNA, 23 bp, Homo sapiens
gctgtattttgaaaatgctttag

SEQ ID NO: 1498 - DNA, 23 bp, Homo sapiens
gctttagaaaggttttgagcatg

SEQ ID NO: 1499 - DNA, 23 bp, Homo sapiens
ttttgagcatgggttcatcctat

SEQ ID NO: 1500 - DNA, 23 bp, Homo sapiens
ttcatcctattctttcgaaagaa

SEQ ID NO: 1501 - DNA, 23 bp, Homo sapiens
aaaatgagtgataaatacaaggc

SEQ ID NO: 1502 - DNA, 23 bp, Homo sapiens
aaatacaaggcccagatgtggtt

SEQ ID NO: 1503 - DNA, 23 bp, Homo sapiens
cagatgtggttgcataaggtttt

SEQ ID NO: 1504 - DNA, 23 bp, Homo sapiens
tatgcatgtttgttgtatacttc

SEQ ID NO: 1505 - DNA, 23 bp, Homo sapiens
acttccttatgcttctttcaaat

SEQ ID NO: 1506 - DNA, 23 bp, Homo sapiens
tctttcaaattgtgtgtgctctg

SEQ ID NO: 1507 - DNA, 23 bp, Homo sapiens
ctctgcttcaatgtagtcagaat

SEQ ID NO: 1508 - DNA, 23 bp, Homo sapiens
gtagtcagaattagctgcttcta

SEQ ID NO: 1509 - DNA, 23 bp, Homo sapiens
gctgcttctatgtttcatagttg

SEQ ID NO: 1510 - DNA, 23 bp, Homo sapiens
agatgtttccttgccttgttgat

SEQ ID NO: 1511 - DNA, 23 bp, Homo sapiens
gatgtggacatgagccatttgag

SEQ ID NO: 1512 - DNA, 23 bp, Homo sapiens
gaacggaaataaaggagttattt

SEQ ID NO: 1513 - DNA, 23 bp, Homo sapiens
ggagttatttgtaatgactaaaa

SEQ ID NO: 1514 - RNA, 21 nt, Artificial Sequence
  Description of Artificial Sequence: Synthetic oligonucleotide
gcagaugcgauccagcggcua

SEQ ID NO: 1515 - RNA, 21 nt, Artificial Sequence
  Description of Artificial Sequence: Synthetic oligonucleotide
cggugguagcagcugguacca

SEQ ID NO: 1516 - RNA, 21 nt, Artificial Sequence
  Description of Artificial Sequence: Synthetic oligonucleotide
gcgcugaugaugggugaggaa

SEQ ID NO: 1517 - RNA, 21 nt, Artificial Sequence
  Description of Artificial Sequence: Synthetic oligonucleotide
ugccugcgaacucugaggaga

SEQ ID NO: 1518 - RNA, 21 nt, Artificial Sequence
  Description of Artificial Sequence: Synthetic oligonucleotide
ggacgcugcaaacuugcgcaa

SEQ ID NO: 1519 - RNA, 21 nt, Artificial Sequence
  Description of Artificial Sequence: Synthetic oligonucleotide
gcugggauucacgcccagaaa

SEQ ID NO: 1520 - RNA, 21 nt, Artificial Sequence
  Description of Artificial Sequence: Synthetic oligonucleotide
gcccagaaguucagcaggcaa

SEQ ID NO: 1521 - RNA, 21 nt, Artificial Sequence
  Description of Artificial Sequence: Synthetic oligonucleotide
gcagacaguccgaagccuuca

SEQ ID NO: 1522 - RNA, 21 nt, Artificial Sequence
  Description of Artificial Sequence: Synthetic oligonucleotide
cagcggagagauagcuugaga

SEQ ID NO: 1523 - RNA, 21 nt, Artificial Sequence
  Description of Artificial Sequence: Synthetic oligonucleotide
ugagggugcgcaagacggcaa

SEQ ID NO: 1524 - RNA, 21 nt, Artificial Sequence
  Description of Artificial Sequence: Synthetic oligonucleotide
gggcagaagagcuuggaggaa

SEQ ID NO: 1525 - RNA, 21 nt, Artificial Sequence
  Description of Artificial Sequence: Synthetic oligonucleotide
gagccaaaaggaacgcaaaaa

SEQ ID NO: 1526 - RNA, 21 nt, Artificial Sequence
  Description of Artificial Sequence: Synthetic oligonucleotide
aaaggcggccaggacagcgua

SEQ ID NO: 1527 - RNA, 21 nt, Artificial Sequence
  Description of Artificial Sequence: Synthetic oligonucleotide
ccgccguucucagccuuaaaa

SEQ ID NO: 1528 - RNA, 21 nt, Artificial Sequence
  Description of Artificial Sequence: Synthetic oligonucleotide
ccuuaaaaguugcagagauua

SEQ ID NO: 1529 - RNA, 21 nt, Artificial Sequence
  Description of Artificial Sequence: Synthetic oligonucleotide
gacgguacccaacugccacca

SEQ ID NO: 1530 - RNA, 21 nt, Artificial Sequence
  Description of Artificial Sequence: Synthetic oligonucleotide
cugccaccucccuucaaccaa

SEQ ID NO: 1531 - RNA, 21 nt, Artificial Sequence
  Description of Artificial Sequence: Synthetic oligonucleotide
ucaaccauaguaguuccucua

SEQ ID NO: 1532 - RNA, 21 nt, Artificial Sequence
  Description of Artificial Sequence: Synthetic oligonucleotide
guuccucuguaccgagcgcaa

SEQ ID NO: 1533 - RNA, 21 nt, Artificial Sequence
  Description of Artificial Sequence: Synthetic oligonucleotide
cgagcgcagcgagcuacagaa

SEQ ID NO: 1534 - RNA, 21 nt, Artificial Sequence
  Description of Artificial Sequence: Synthetic oligonucleotide
ggcucaaggucccagccagua

SEQ ID NO: 1535 - RNA, 21 nt, Artificial Sequence
  Description of Artificial Sequence: Synthetic oligonucleotide
gccagugagcccagugugcua

SEQ ID NO: 1536 - RNA, 21 nt, Artificial Sequence
  Description of Artificial Sequence: Synthetic oligonucleotide
agugugcuugagugucucuga

SEQ ID NO: 1537 - RNA, 21 nt, Artificial Sequence
  Description of Artificial Sequence: Synthetic oligonucleotide
ggucuguuucauuuagacuca

SEQ ID NO: 1538 - RNA, 21 nt, Artificial Sequence
  Description of Artificial Sequence: Synthetic oligonucleotide
cugcucgccuccgugcaguua

SEQ ID NO: 1539 - RNA, 21 nt, Artificial Sequence
  Description of Artificial Sequence: Synthetic oligonucleotide
gaaagcaagagacuugcgcga

SEQ ID NO: 1540 - RNA, 21 nt, Artificial Sequence
  Description of Artificial Sequence: Synthetic oligonucleotide
gcgcgcacgcacaguccucua

SEQ ID NO: 1541 - RNA, 21 nt, Artificial Sequence
  Description of Artificial Sequence: Synthetic oligonucleotide
uccucuggagaucagguggaa

SEQ ID NO: 1542 - RNA, 21 nt, Artificial Sequence
  Description of Artificial Sequence: Synthetic oligonucleotide
aggagccgcuggguaccaaga

SEQ ID NO: 1543 - RNA, 21 nt, Artificial Sequence
  Description of Artificial Sequence: Synthetic oligonucleotide
guaccaaggacuguucagaga

SEQ ID NO: 1544 - RNA, 21 nt, Artificial Sequence
  Description of Artificial Sequence: Synthetic oligonucleotide
ucagagccucuucccaucuca

SEQ ID NO: 1545 - RNA, 21 nt, Artificial Sequence
  Description of Artificial Sequence: Synthetic oligonucleotide
ccggagagcaguguaaacgga

SEQ ID NO: 1546 - RNA, 21 nt, Artificial Sequence
  Description of Artificial Sequence: Synthetic oligonucleotide
gggagccaucgggcuccugua

SEQ ID NO: 1547 - RNA, 21 nt, Artificial Sequence
  Description of Artificial Sequence: Synthetic oligonucleotide
ugcugcuuuccacggcagcua

SEQ ID NO: 1548 - RNA, 21 nt, Artificial Sequence
  Description of Artificial Sequence: Synthetic oligonucleotide
ccacucagcuacucgcgccua

SEQ ID NO: 1549 - RNA, 21 nt, Artificial Sequence
  Description of Artificial Sequence: Synthetic oligonucleotide
cagaggaagagucuggcagua

SEQ ID NO: 1550 - RNA, 21 nt, Artificial Sequence
  Description of Artificial Sequence: Synthetic oligonucleotide
cuggcaguugacuucguggua

SEQ ID NO: 1551 - RNA, 21 nt, Artificial Sequence
  Description of Artificial Sequence: Synthetic oligonucleotide
uucguggugcccucgcucuua

SEQ ID NO: 1552 - RNA, 21 nt, Artificial Sequence
  Description of Artificial Sequence: Synthetic oligonucleotide
ucgcucuuccgugucuacgca

SEQ ID NO: 1553 - RNA, 21 nt, Artificial Sequence
  Description of Artificial Sequence: Synthetic oligonucleotide
gucuacgcccgggaccuacua

SEQ ID NO: 1554 - RNA, 21 nt, Artificial Sequence
  Description of Artificial Sequence: Synthetic oligonucleotide
gaccuacugcugccaccauca

SEQ ID NO: 1555 - RNA, 21 nt, Artificial Sequence
  Description of Artificial Sequence: Synthetic oligonucleotide
cucggagcugaaggcuggcaa

SEQ ID NO: 1556 - RNA, 21 nt, Artificial Sequence
  Description of Artificial Sequence: Synthetic oligonucleotide
gcuguccagggugcugaagga

SEQ ID NO: 1557 - RNA, 21 nt, Artificial Sequence
  Description of Artificial Sequence: Synthetic oligonucleotide
cgugccaagcaguuggugcua

SEQ ID NO: 1558 - RNA, 21 nt, Artificial Sequence
  Description of Artificial Sequence: Synthetic oligonucleotide
uuggugcuggagcugggcgaa

SEQ ID NO: 1559 - RNA, 21 nt, Artificial Sequence
  Description of Artificial Sequence: Synthetic oligonucleotide
gaggcgaucuuggaggguuga

SEQ ID NO: 1560 - RNA, 21 nt, Artificial Sequence
  Description of Artificial Sequence: Synthetic oligonucleotide
cugcuccaguucaaucucaga

SEQ ID NO: 1561 - RNA, 21 nt, Artificial Sequence
  Description of Artificial Sequence: Synthetic oligonucleotide
cagcgagcuguucaguuggua

SEQ ID NO: 1562 - RNA, 21 nt, Artificial Sequence
  Description of Artificial Sequence: Synthetic oligonucleotide
uugguggauucgccaaggcga

SEQ ID NO: 1563 - RNA, 21 nt, Artificial Sequence
  Description of Artificial Sequence: Synthetic oligonucleotide
gggcgacugaggauccgccua

SEQ ID NO: 1564 - RNA, 21 nt, Artificial Sequence
  Description of Artificial Sequence: Synthetic oligonucleotide
auccgccugaugcccgagaaa

SEQ ID NO: 1565 - RNA, 21 nt, Artificial Sequence
  Description of Artificial Sequence: Synthetic oligonucleotide
ccgagaagaaggcgucggaaa

SEQ ID NO: 1566 - RNA, 21 nt, Artificial Sequence
  Description of Artificial Sequence: Synthetic oligonucleotide
gggcagagagggaaggcugua

SEQ ID NO: 1567 - RNA, 21 nt, Artificial Sequence
  Description of Artificial Sequence: Synthetic oligonucleotide
ggcuguccgcggcaauucgca

SEQ ID NO: 1568 - RNA, 21 nt, Artificial Sequence
  Description of Artificial Sequence: Synthetic oligonucleotide
ccuucucuuccagaucuucga

SEQ ID NO: 1569 - RNA, 21 nt, Artificial Sequence
  Description of Artificial Sequence: Synthetic oligonucleotide
acuggucauagcuccuuggaa

SEQ ID NO: 1570 - RNA, 21 nt, Artificial Sequence
  Description of Artificial Sequence: Synthetic oligonucleotide
ccuuggaaucaccaacaaaca

SEQ ID NO: 1571 - RNA, 21 nt, Artificial Sequence
  Description of Artificial Sequence: Synthetic oligonucleotide
caaacaugccuucuccuucua

SEQ ID NO: 1572 - RNA, 21 nt, Artificial Sequence
  Description of Artificial Sequence: Synthetic oligonucleotide
cuucuccugauuauuuuacaa

SEQ ID NO: 1573 - RNA, 21 nt, Artificial Sequence
  Description of Artificial Sequence: Synthetic oligonucleotide
uuuuacauggaaucucaccua

SEQ ID NO: 1574 - RNA, 21 nt, Artificial Sequence
  Description of Artificial Sequence: Synthetic oligonucleotide
ucucaccuggauaaugaaaga

SEQ ID NO: 1575 - RNA, 21 nt, Artificial Sequence
  Description of Artificial Sequence: Synthetic oligonucleotide
augaaagacuccuucccuuua

SEQ ID NO: 1576 - RNA, 21 nt, Artificial Sequence
  Description of Artificial Sequence: Synthetic oligonucleotide
uucccuuuccugucucaucga

SEQ ID NO: 1577 - RNA, 21 nt, Artificial Sequence
  Description of Artificial Sequence: Synthetic oligonucleotide
caucgcagccgauauggucua

SEQ ID NO: 1578 - RNA, 21 nt, Artificial Sequence
  Description of Artificial Sequence: Synthetic oligonucleotide
ggagugcagcuuugacuucca

SEQ ID NO: 1579 - RNA, 21 nt, Artificial Sequence
  Description of Artificial Sequence: Synthetic oligonucleotide
ccacugcaugaccucaggaaa

SEQ ID NO: 1580 - RNA, 21 nt, Artificial Sequence
  Description of Artificial Sequence: Synthetic oligonucleotide
caggaaccagagcugguccua

SEQ ID NO: 1581 - RNA, 21 nt, Artificial Sequence
  Description of Artificial Sequence: Synthetic oligonucleotide
ucccagauggacuugcuggaa

SEQ ID NO: 1582 - RNA, 21 nt, Artificial Sequence
  Description of Artificial Sequence: Synthetic oligonucleotide
agagcguucuaaggagaugca

SEQ ID NO: 1583 - RNA, 21 nt, Artificial Sequence
  Description of Artificial Sequence: Synthetic oligonucleotide
augcccagaggcuccuuucua

SEQ ID NO: 1584 - RNA, 21 nt, Artificial Sequence
  Description of Artificial Sequence: Synthetic oligonucleotide
uccuuucuccuucucaacaca

SEQ ID NO: 1585 - RNA, 21 nt, Artificial Sequence
  Description of Artificial Sequence: Synthetic oligonucleotide
agcugacuccaagcacaccaa

SEQ ID NO: 1586 - RNA, 21 nt, Artificial Sequence
  Description of Artificial Sequence: Synthetic oligonucleotide
gcacaccauccugaguccgua

SEQ ID NO: 1587 - RNA, 21 nt, Artificial Sequence
  Description of Artificial Sequence: Synthetic oligonucleotide
gaguccguggaugaggagcaa

SEQ ID NO: 1588 - RNA, 21 nt, Artificial Sequence
  Description of Artificial Sequence: Synthetic oligonucleotide
cagcagugagcacugcacaca

SEQ ID NO: 1589 - RNA, 21 nt, Artificial Sequence
  Description of Artificial Sequence: Synthetic oligonucleotide
ugcacacuggccgucucggua

SEQ ID NO: 1590 - RNA, 21 nt, Artificial Sequence
  Description of Artificial Sequence: Synthetic oligonucleotide
ucggugcacaggcaccugcaa

SEQ ID NO: 1591 - RNA, 21 nt, Artificial Sequence
  Description of Artificial Sequence: Synthetic oligonucleotide
cucuggaagguacauugccca

SEQ ID NO: 1592 - RNA, 21 nt, Artificial Sequence
  Description of Artificial Sequence: Synthetic oligonucleotide
gcugcaagagagauccuccua

SEQ ID NO: 1593 - RNA, 21 nt, Artificial Sequence
  Description of Artificial Sequence: Synthetic oligonucleotide
auccuccugaugcccacucca

SEQ ID NO: 1594 - RNA, 21 nt, Artificial Sequence
  Description of Artificial Sequence: Synthetic oligonucleotide
cagggaagcaugguuggacaa

SEQ ID NO: 1595 - RNA, 21 nt, Artificial Sequence
  Description of Artificial Sequence: Synthetic oligonucleotide
uuggacagugcuccagggaaa

SEQ ID NO: 1596 - RNA, 21 nt, Artificial Sequence
  Description of Artificial Sequence: Synthetic oligonucleotide
ccagggaagaaucgggcguca

SEQ ID NO: 1597 - RNA, 21 nt, Artificial Sequence
  Description of Artificial Sequence: Synthetic oligonucleotide
ccagacaacccauuucgagua

SEQ ID NO: 1598 - RNA, 21 nt, Artificial Sequence
  Description of Artificial Sequence: Synthetic oligonucleotide
ucgaguggcccuggaauacaa

SEQ ID NO: 1599 - RNA, 21 nt, Artificial Sequence
  Description of Artificial Sequence: Synthetic oligonucleotide
ggaauacaucuccaguggaaa

SEQ ID NO: 1600 - RNA, 21 nt, Artificial Sequence
  Description of Artificial Sequence: Synthetic oligonucleotide
caguggaaaccgcagcuugua

SEQ ID NO: 1601 - RNA, 21 nt, Artificial Sequence
  Description of Artificial Sequence: Synthetic oligonucleotide
cugcaguggacuucuuugcca

SEQ ID NO: 1602 - RNA, 21 nt, Artificial Sequence
  Description of Artificial Sequence: Synthetic oligonucleotide
ccugaagaacugcagugaaga

SEQ ID NO: 1603 - RNA, 21 nt, Artificial Sequence
  Description of Artificial Sequence: Synthetic oligonucleotide
ggcuccaagauggcccugcaa

SEQ ID NO: 1604 - RNA, 21 nt, Artificial Sequence
  Description of Artificial Sequence: Synthetic oligonucleotide
cuccuucacuuguuggaauga

SEQ ID NO: 1605 - RNA, 21 nt, Artificial Sequence
  Description of Artificial Sequence: Synthetic oligonucleotide
acaguccuccagcuugggcaa

SEQ ID NO: 1606 - RNA, 21 nt, Artificial Sequence
  Description of Artificial Sequence: Synthetic oligonucleotide
cugugacuuccaccaggacua

SEQ ID NO: 1607 - RNA, 21 nt, Artificial Sequence
  Description of Artificial Sequence: Synthetic oligonucleotide
caggacugugcccagggagaa

SEQ ID NO: 1608 - RNA, 21 nt, Artificial Sequence
  Description of Artificial Sequence: Synthetic oligonucleotide
cagggagaagaugagagccaa

SEQ ID NO: 1609 - RNA, 21 nt, Artificial Sequence
  Description of Artificial Sequence: Synthetic oligonucleotide
agccagaugugccggaaacua

SEQ ID NO: 1610 - RNA, 21 nt, Artificial Sequence
  Description of Artificial Sequence: Synthetic oligonucleotide
cggaaacugccuguggguuua

SEQ ID NO: 1611 - RNA, 21 nt, Artificial Sequence
  Description of Artificial Sequence: Synthetic oligonucleotide
ugcaacuuugaagauggcuua

SEQ ID NO: 1612 - RNA, 21 nt, Artificial Sequence
  Description of Artificial Sequence: Synthetic oligonucleotide
gauggcuucuguggcuggaca

SEQ ID NO: 1613 - RNA, 21 nt, Artificial Sequence
  Description of Artificial Sequence: Synthetic oligonucleotide
acccaaggcacacugucacca

SEQ ID NO: 1614 - RNA, 21 nt, Artificial Sequence
  Description of Artificial Sequence: Synthetic oligonucleotide
acuccucaauggcaggucaga

SEQ ID NO: 1615 - RNA, 21 nt, Artificial Sequence
  Description of Artificial Sequence: Synthetic oligonucleotide
gacccuaaaggaugcccggua

SEQ ID NO: 1616 - RNA, 21 nt, Artificial Sequence
  Description of Artificial Sequence: Synthetic oligonucleotide
gcccgguuccaggaccaccaa

SEQ ID NO: 1617 - RNA, 21 nt, Artificial Sequence
  Description of Artificial Sequence: Synthetic oligonucleotide
gaccaccaagaccaugcucua

SEQ ID NO: 1618 - RNA, 21 nt, Artificial Sequence
  Description of Artificial Sequence: Synthetic oligonucleotide
ugcucuauugcucaguaccaa

SEQ ID NO: 1619 - RNA, 21 nt, Artificial Sequence
  Description of Artificial Sequence: Synthetic oligonucleotide
gcuucugaaagugcuacagua

SEQ ID NO: 1620 - RNA, 21 nt, Artificial Sequence
  Description of Artificial Sequence: Synthetic oligonucleotide
ccagugcuacguuuccugcaa

SEQ ID NO: 1621 - RNA, 21 nt, Artificial Sequence
  Description of Artificial Sequence: Synthetic oligonucleotide
accgaucaagagcucuccaua

SEQ ID NO: 1622 - RNA, 21 nt, Artificial Sequence
  Description of Artificial Sequence: Synthetic oligonucleotide
cucuccaugugagcuccgaaa

SEQ ID NO: 1623 - RNA, 21 nt, Artificial Sequence
  Description of Artificial Sequence: Synthetic oligonucleotide
gcuccgaauguccuggcucaa

SEQ ID NO: 1624 - RNA, 21 nt, Artificial Sequence
  Description of Artificial Sequence: Synthetic oligonucleotide
uggcucauucguggagucuua

SEQ ID NO: 1625 - RNA, 21 nt, Artificial Sequence
  Description of Artificial Sequence: Synthetic oligonucleotide
gaaacguguccuuggugcuaa

SEQ ID NO: 1626 - RNA, 21 nt, Artificial Sequence
  Description of Artificial Sequence: Synthetic oligonucleotide
gugcuaguggagaacaaaaca

SEQ ID NO: 1627 - RNA, 21 nt, Artificial Sequence
  Description of Artificial Sequence: Synthetic oligonucleotide
gggaaggagcaaggcaggaua

SEQ ID NO: 1628 - RNA, 21 nt, Artificial Sequence
  Description of Artificial Sequence: Synthetic oligonucleotide
ggcaggauggucuggcaugua

SEQ ID NO: 1629 - RNA, 21 nt, Artificial Sequence
  Description of Artificial Sequence: Synthetic oligonucleotide
cgccuaugaaggcuugagcca

SEQ ID NO: 1630 - RNA, 21 nt, Artificial Sequence
  Description of Artificial Sequence: Synthetic oligonucleotide
uugagccuguggcaguggaua

SEQ ID NO: 1631 - RNA, 21 nt, Artificial Sequence
  Description of Artificial Sequence: Synthetic oligonucleotide
caguggaugguguugccucua

SEQ ID NO: 1632 - RNA, 21 nt, Artificial Sequence
  Description of Artificial Sequence: Synthetic oligonucleotide
cucgaugugucugacagguua

SEQ ID NO: 1633 - RNA, 21 nt, Artificial Sequence
  Description of Artificial Sequence: Synthetic oligonucleotide
cuggcugcagauggucgcaua

SEQ ID NO: 1634 - RNA, 21 nt, Artificial Sequence
  Description of Artificial Sequence: Synthetic oligonucleotide
auccagagccaucguggcuua

SEQ ID NO: 1635 - RNA, 21 nt, Artificial Sequence
  Description of Artificial Sequence: Synthetic oligonucleotide
guggcuuuugacaauaucuca

SEQ ID NO: 1636 - RNA, 21 nt, Artificial Sequence
  Description of Artificial Sequence: Synthetic oligonucleotide
aauaucuccaucagccuggaa

SEQ ID NO: 1637 - RNA, 21 nt, Artificial Sequence
  Description of Artificial Sequence: Synthetic oligonucleotide
agccuggacugcuaccucaca

SEQ ID NO: 1638 - RNA, 21 nt, Artificial Sequence
  Description of Artificial Sequence: Synthetic oligonucleotide
uaccucaccauuagcggagaa

SEQ ID NO: 1639 - RNA, 21 nt, Artificial Sequence
  Description of Artificial Sequence: Synthetic oligonucleotide
ggacaagauccugcagaauaa

SEQ ID NO: 1640 - RNA, 21 nt, Artificial Sequence
  Description of Artificial Sequence: Synthetic oligonucleotide
agaauacagcacccaaaucaa

SEQ ID NO: 1641 - RNA, 21 nt, Artificial Sequence
  Description of Artificial Sequence: Synthetic oligonucleotide
ccaaaucaagaaaccuguuua

SEQ ID NO: 1642 - RNA, 21 nt, Artificial Sequence
  Description of Artificial Sequence: Synthetic oligonucleotide
ccuguuugagagaaacccaaa

SEQ ID NO: 1643 - RNA, 21 nt, Artificial Sequence
  Description of Artificial Sequence: Synthetic oligonucleotide
cccaaacaaggagcugaaaca

SEQ ID NO: 1644 - RNA, 21 nt, Artificial Sequence
  Description of Artificial Sequence: Synthetic oligonucleotide
gaaaauucaccaagacagaca

SEQ ID NO: 1645 - RNA, 21 nt, Artificial Sequence
  Description of Artificial Sequence: Synthetic oligonucleotide
cuuugacccuacaguucauua

SEQ ID NO: 1646 - RNA, 21 nt, Artificial Sequence
  Description of Artificial Sequence: Synthetic oligonucleotide
aguucauuggcuguucaccaa

SEQ ID NO: 1647 - RNA, 21 nt, Artificial Sequence
  Description of Artificial Sequence: Synthetic oligonucleotide
gcacagugcaacaacgccuaa

SEQ ID NO: 1648 - RNA, 21 nt, Artificial Sequence
  Description of Artificial Sequence: Synthetic oligonucleotide
cuaccagaacuccaaccugaa

SEQ ID NO: 1649 - RNA, 21 nt, Artificial Sequence
  Description of Artificial Sequence: Synthetic oligonucleotide
caaccugagcguggaggugga

SEQ ID NO: 1650 - RNA, 21 nt, Artificial Sequence
  Description of Artificial Sequence: Synthetic oligonucleotide
cauccagaucuggaaggugca

SEQ ID NO: 1651 - RNA, 21 nt, Artificial Sequence
  Description of Artificial Sequence: Synthetic oligonucleotide
gaaggugccagccaccgacaa

SEQ ID NO: 1652 - RNA, 21 nt, Artificial Sequence
  Description of Artificial Sequence: Synthetic oligonucleotide
caccgacaccuacagcaucua

SEQ ID NO: 1653 - RNA, 21 nt, Artificial Sequence
  Description of Artificial Sequence: Synthetic oligonucleotide
ucucgggcuacggagcugcua

SEQ ID NO: 1654 - RNA, 21 nt, Artificial Sequence
  Description of Artificial Sequence: Synthetic oligonucleotide
ggcgggaaaggcgggaagaaa

SEQ ID NO: 1655 - RNA, 21 nt, Artificial Sequence
  Description of Artificial Sequence: Synthetic oligonucleotide
gggaagaacaccaugaugcga

SEQ ID NO: 1656 - RNA, 21 nt, Artificial Sequence
  Description of Artificial Sequence: Synthetic oligonucleotide
gaugcggucccacggcgugua

SEQ ID NO: 1657 - RNA, 21 nt, Artificial Sequence
  Description of Artificial Sequence: Synthetic oligonucleotide
ggcgugucugugcugggcaua

SEQ ID NO: 1658 - RNA, 21 nt, Artificial Sequence
  Description of Artificial Sequence: Synthetic oligonucleotide
ggcaucuucaaccuggagaaa

SEQ ID NO: 1659 - RNA, 21 nt, Artificial Sequence
  Description of Artificial Sequence: Synthetic oligonucleotide
aaggaugacaugcuguacaua

SEQ ID NO: 1660 - RNA, 21 nt, Artificial Sequence
  Description of Artificial Sequence: Synthetic oligonucleotide
cuguacauccugguugggcaa

SEQ ID NO: 1661 - RNA, 21 nt, Artificial Sequence
  Description of Artificial Sequence: Synthetic oligonucleotide
guugggcagcagggagaggaa

SEQ ID NO: 1662 - RNA, 21 nt, Artificial Sequence
  Description of Artificial Sequence: Synthetic oligonucleotide
aguacaaaccaguuaauccaa

SEQ ID NO: 1663 - RNA, 21 nt, Artificial Sequence
  Description of Artificial Sequence: Synthetic oligonucleotide
uuaauccagaaagucugcaua

SEQ ID NO: 1664 - RNA, 21 nt, Artificial Sequence
  Description of Artificial Sequence: Synthetic oligonucleotide
ugcauuggagagaacaaugua

SEQ ID NO: 1665 - RNA, 21 nt, Artificial Sequence
  Description of Artificial Sequence: Synthetic oligonucleotide
aaugugauagaagaagaaaua

SEQ ID NO: 1666 - RNA, 21 nt, Artificial Sequence
  Description of Artificial Sequence: Synthetic oligonucleotide
auccgugugaacagaagcgua

SEQ ID NO: 1667 - RNA, 21 nt, Artificial Sequence
  Description of Artificial Sequence: Synthetic oligonucleotide
gaagcgugcaugagugggcaa

SEQ ID NO: 1668 - RNA, 21 nt, Artificial Sequence
  Description of Artificial Sequence: Synthetic oligonucleotide
caccuacguauuuaagaugaa

SEQ ID NO: 1669 - RNA, 21 nt, Artificial Sequence
  Description of Artificial Sequence: Synthetic oligonucleotide
uaagaugaaggauggagugca

SEQ ID NO: 1670 - RNA, 21 nt, Artificial Sequence
  Description of Artificial Sequence: Synthetic oligonucleotide
cugaucauugcagccggagga

SEQ ID NO: 1671 - RNA, 21 nt, Artificial Sequence
  Description of Artificial Sequence: Synthetic oligonucleotide
ccaagacagacacguuccaca

SEQ ID NO: 1672 - RNA, 21 nt, Artificial Sequence
  Description of Artificial Sequence: Synthetic oligonucleotide
agagagacuggagaauaacua

SEQ ID NO: 1673 - RNA, 21 nt, Artificial Sequence
  Description of Artificial Sequence: Synthetic oligonucleotide
gaauaacuccucgguucuaga

SEQ ID NO: 1674 - RNA, 21 nt, Artificial Sequence
  Description of Artificial Sequence: Synthetic oligonucleotide
agggcuaaacggcaauuccga

SEQ ID NO: 1675 - RNA, 21 nt, Artificial Sequence
  Description of Artificial Sequence: Synthetic oligonucleotide
auuccggagccgcagguggua

SEQ ID NO: 1676 - RNA, 21 nt, Artificial Sequence
  Description of Artificial Sequence: Synthetic oligonucleotide
gugguggagguggcuggaaua

SEQ ID NO: 1677 - RNA, 21 nt, Artificial Sequence
  Description of Artificial Sequence: Synthetic oligonucleotide
uggaaugauaacacuuccuua

SEQ ID NO: 1678 - RNA, 21 nt, Artificial Sequence
  Description of Artificial Sequence: Synthetic oligonucleotide
acuuccuugcucugggccgga

SEQ ID NO: 1679 - RNA, 21 nt, Artificial Sequence
  Description of Artificial Sequence: Synthetic oligonucleotide
ggccggaaaaucuuugcagga

SEQ ID NO: 1680 - RNA, 21 nt, Artificial Sequence
  Description of Artificial Sequence: Synthetic oligonucleotide
ugcaggagggugccaccggaa

SEQ ID NO: 1681 - RNA, 21 nt, Artificial Sequence
  Description of Artificial Sequence: Synthetic oligonucleotide
ccaccggaggacauuccugca

SEQ ID NO: 1682 - RNA, 21 nt, Artificial Sequence
  Description of Artificial Sequence: Synthetic oligonucleotide
aggcggaggauauauaggcga

SEQ ID NO: 1683 - RNA, 21 nt, Artificial Sequence
  Description of Artificial Sequence: Synthetic oligonucleotide
augcagccucaaacaaugaca

SEQ ID NO: 1684 - RNA, 21 nt, Artificial Sequence
  Description of Artificial Sequence: Synthetic oligonucleotide
guuuccuucaucaguccacua

SEQ ID NO: 1685 - RNA, 21 nt, Artificial Sequence
  Description of Artificial Sequence: Synthetic oligonucleotide
cacugggcauccuguacacca

SEQ ID NO: 1686 - RNA, 21 nt, Artificial Sequence
  Description of Artificial Sequence: Synthetic oligonucleotide
cagcuuuaaaagugauggaaa

SEQ ID NO: 1687 - RNA, 21 nt, Artificial Sequence
  Description of Artificial Sequence: Synthetic oligonucleotide
uaagcauuaucuaaacugcaa

SEQ ID NO: 1688 - RNA, 21 nt, Artificial Sequence
  Description of Artificial Sequence: Synthetic oligonucleotide
ugcagucacugugagguagaa

SEQ ID NO: 1689 - RNA, 21 nt, Artificial Sequence
  Description of Artificial Sequence: Synthetic oligonucleotide
guagacgaaugucacauggaa

SEQ ID NO: 1690 - RNA, 21 nt, Artificial Sequence
  Description of Artificial Sequence: Synthetic oligonucleotide
cccugaaagccacaaggucaa

SEQ ID NO: 1691 - RNA, 21 nt, Artificial Sequence
  Description of Artificial Sequence: Synthetic oligonucleotide
aaggucaucugcuucugugaa

SEQ ID NO: 1692 - RNA, 21 nt, Artificial Sequence
  Description of Artificial Sequence: Synthetic oligonucleotide
uucugugaccacgggacggua

SEQ ID NO: 1693 - RNA, 21 nt, Artificial Sequence
  Description of Artificial Sequence: Synthetic oligonucleotide
gcuggcugaggauggcgucua

SEQ ID NO: 1694 - RNA, 21 nt, Artificial Sequence
  Description of Artificial Sequence: Synthetic oligonucleotide
gucuccugcauugugucacca

SEQ ID NO: 1695 - RNA, 21 nt, Artificial Sequence
  Description of Artificial Sequence: Synthetic oligonucleotide
ccacaccugccacucucgcua

SEQ ID NO: 1696 - RNA, 21 nt, Artificial Sequence
  Description of Artificial Sequence: Synthetic oligonucleotide
ucgcugauccucucuguggua

SEQ ID NO: 1697 - RNA, 21 nt, Artificial Sequence
  Description of Artificial Sequence: Synthetic oligonucleotide
ucuguggugaccucugcccua

SEQ ID NO: 1698 - RNA, 21 nt, Artificial Sequence
  Description of Artificial Sequence: Synthetic oligonucleotide
guccuggcuuucuccggcaua

SEQ ID NO: 1699 - RNA, 21 nt, Artificial Sequence
  Description of Artificial Sequence: Synthetic oligonucleotide
uccggcaucaugauuguguaa

SEQ ID NO: 1700 - RNA, 21 nt, Artificial Sequence
  Description of Artificial Sequence: Synthetic oligonucleotide
uaccgccggaagcaccaggaa

SEQ ID NO: 1701 - RNA, 21 nt, Artificial Sequence
  Description of Artificial Sequence: Synthetic oligonucleotide
caccaggagcugcaagccaua

SEQ ID NO: 1702 - RNA, 21 nt, Artificial Sequence
  Description of Artificial Sequence: Synthetic oligonucleotide
caagccaugcagauggagcua

SEQ ID NO: 1703 - RNA, 21 nt, Artificial Sequence
  Description of Artificial Sequence: Synthetic oligonucleotide
auggagcugcagagcccugaa

SEQ ID NO: 1704 - RNA, 21 nt, Artificial Sequence
  Description of Artificial Sequence: Synthetic oligonucleotide
ccugaguacaagcugagcaaa

SEQ ID NO: 1705 - RNA, 21 nt, Artificial Sequence
  Description of Artificial Sequence: Synthetic oligonucleotide
gcuccgcaccucgaccaucaa

SEQ ID NO: 1706 - RNA, 21 nt, Artificial Sequence
  Description of Artificial Sequence: Synthetic oligonucleotide
accaucaugaccgacuacaaa

SEQ ID NO: 1707 - RNA, 21 nt, Artificial Sequence
  Description of Artificial Sequence: Synthetic oligonucleotide
caacuacugcuuugcuggcaa

SEQ ID NO: 1708 - RNA, 21 nt, Artificial Sequence
  Description of Artificial Sequence: Synthetic oligonucleotide
ugcuggcaagaccuccuccaa

SEQ ID NO: 1709 - RNA, 21 nt, Artificial Sequence
  Description of Artificial Sequence: Synthetic oligonucleotide
uccuccaucagugaccugaaa

SEQ ID NO: 1710 - RNA, 21 nt, Artificial Sequence
  Description of Artificial Sequence: Synthetic oligonucleotide
ugaaggaggugccgcggaaaa

SEQ ID NO: 1711 - RNA, 21 nt, Artificial Sequence
  Description of Artificial Sequence: Synthetic oligonucleotide
gagguguaugaaggccaggua

SEQ ID NO: 1712 - RNA, 21 nt, Artificial Sequence
  Description of Artificial Sequence: Synthetic oligonucleotide
gguguccggaaugcccaacga

SEQ ID NO: 1713 - RNA, 21 nt, Artificial Sequence
  Description of Artificial Sequence: Synthetic oligonucleotide
gcugugaagacgcugccugaa

SEQ ID NO: 1714 - RNA, 21 nt, Artificial Sequence
  Description of Artificial Sequence: Synthetic oligonucleotide
ugugcucugaacaggacgaaa

SEQ ID NO: 1715 - RNA, 21 nt, Artificial Sequence
  Description of Artificial Sequence: Synthetic oligonucleotide
ggacgaacuggauuuccucaa

SEQ ID NO: 1716 - RNA, 21 nt, Artificial Sequence
  Description of Artificial Sequence: Synthetic oligonucleotide
uuccucauggaagcccugaua

SEQ ID NO: 1717 - RNA, 21 nt, Artificial Sequence
  Description of Artificial Sequence: Synthetic oligonucleotide
gaucaucagcaaauucaacca

SEQ ID NO: 1718 - RNA, 21 nt, Artificial Sequence
  Description of Artificial Sequence: Synthetic oligonucleotide
caaccaccagaacauuguuca

SEQ ID NO: 1719 - RNA, 21 nt, Artificial Sequence
  Description of Artificial Sequence: Synthetic oligonucleotide
cgguucauccugcuggagcua

SEQ ID NO: 1720 - RNA, 21 nt, Artificial Sequence
  Description of Artificial Sequence: Synthetic oligonucleotide
agaccucaaguccuuccucca

SEQ ID NO: 1721 - RNA, 21 nt, Artificial Sequence
  Description of Artificial Sequence: Synthetic oligonucleotide
augcuggaccuucugcacgua

SEQ ID NO: 1722 - RNA, 21 nt, Artificial Sequence
  Description of Artificial Sequence: Synthetic oligonucleotide
ucgggacauugccuguggcua

SEQ ID NO: 1723 - RNA, 21 nt, Artificial Sequence
  Description of Artificial Sequence: Synthetic oligonucleotide
guggcugucaguauuuggaga

SEQ ID NO: 1724 - RNA, 21 nt, Artificial Sequence
  Description of Artificial Sequence: Synthetic oligonucleotide
uggaggaaaaccacuucauca

SEQ ID NO: 1725 - RNA, 21 nt, Artificial Sequence
  Description of Artificial Sequence: Synthetic oligonucleotide
ccgagacauugcugccagaaa

SEQ ID NO: 1726 - RNA, 21 nt, Artificial Sequence
  Description of Artificial Sequence: Synthetic oligonucleotide
ugccagaaacugccucuugaa

SEQ ID NO: 1727 - RNA, 21 nt, Artificial Sequence
  Description of Artificial Sequence: Synthetic oligonucleotide
ccucuugaccuguccaggcca

SEQ ID NO: 1728 - RNA, 21 nt, Artificial Sequence
  Description of Artificial Sequence: Synthetic oligonucleotide
gcccuggaagaguggccaaga

SEQ ID NO: 1729 - RNA, 21 nt, Artificial Sequence
  Description of Artificial Sequence: Synthetic oligonucleotide
ggccaagauuggagacuucga

SEQ ID NO: 1730 - RNA, 21 nt, Artificial Sequence
  Description of Artificial Sequence: Synthetic oligonucleotide
cuucgggauggcccgagacaa

SEQ ID NO: 1731 - RNA, 21 nt, Artificial Sequence
  Description of Artificial Sequence: Synthetic oligonucleotide
ccgagacaucuacagggcgaa

SEQ ID NO: 1732 - RNA, 21 nt, Artificial Sequence
  Description of Artificial Sequence: Synthetic oligonucleotide
gagcuacuauagaaagggaga

SEQ ID NO: 1733 - RNA, 21 nt, Artificial Sequence
  Description of Artificial Sequence: Synthetic oligonucleotide
gggaggcugugccaugcugca

SEQ ID NO: 1734 - RNA, 21 nt, Artificial Sequence
  Description of Artificial Sequence: Synthetic oligonucleotide
augcugccaguuaaguggaua

SEQ ID NO: 1735 - RNA, 21 nt, Artificial Sequence
  Description of Artificial Sequence: Synthetic oligonucleotide
gaggccuucauggaaggaaua

SEQ ID NO: 1736 - RNA, 21 nt, Artificial Sequence
  Description of Artificial Sequence: Synthetic oligonucleotide
gaaggaauauucacuucuaaa

SEQ ID NO: 1737 - RNA, 21 nt, Artificial Sequence
  Description of Artificial Sequence: Synthetic oligonucleotide
acuucuaaaacagacacauga

SEQ ID NO: 1738 - RNA, 21 nt, Artificial Sequence
  Description of Artificial Sequence: Synthetic oligonucleotide
acaugguccuuuggagugcua

SEQ ID NO: 1739 - RNA, 21 nt, Artificial Sequence
  Description of Artificial Sequence: Synthetic oligonucleotide
gugcugcuaugggaaaucuua

SEQ ID NO: 1740 - RNA, 21 nt, Artificial Sequence
  Description of Artificial Sequence: Synthetic oligonucleotide
gaaaucuuuucucuuggauaa

SEQ ID NO: 1741 - RNA, 21 nt, Artificial Sequence
  Description of Artificial Sequence: Synthetic oligonucleotide
caaaagcaaccaggaaguuca

SEQ ID NO: 1742 - RNA, 21 nt, Artificial Sequence
  Description of Artificial Sequence: Synthetic oligonucleotide
ggaaguucuggaguuugucaa

SEQ ID NO: 1743 - RNA, 21 nt, Artificial Sequence
  Description of Artificial Sequence: Synthetic oligonucleotide
guuugucaccaguggaggcca

SEQ ID NO: 1744 - RNA, 21 nt, Artificial Sequence
  Description of Artificial Sequence: Synthetic oligonucleotide
ccacccaagaacugcccugga

SEQ ID NO: 1745 - RNA, 21 nt, Artificial Sequence
  Description of Artificial Sequence: Synthetic oligonucleotide
ugcccugggccuguauaccga

SEQ ID NO: 1746 - RNA, 21 nt, Artificial Sequence
  Description of Artificial Sequence: Synthetic oligonucleotide
guauaccggauaaugacucaa

SEQ ID NO: 1747 - RNA, 21 nt, Artificial Sequence
  Description of Artificial Sequence: Synthetic oligonucleotide
gcuggcaacaucagccugaaa

SEQ ID NO: 1748 - RNA, 21 nt, Artificial Sequence
  Description of Artificial Sequence: Synthetic oligonucleotide
agccugaagacaggcccaaca

SEQ ID NO: 1749 - RNA, 21 nt, Artificial Sequence
  Description of Artificial Sequence: Synthetic oligonucleotide
aacuuugccaucauuuuggaa

SEQ ID NO: 1750 - RNA, 21 nt, Artificial Sequence
  Description of Artificial Sequence: Synthetic oligonucleotide
ggagaggauugaauacugcaa

SEQ ID NO: 1751 - RNA, 21 nt, Artificial Sequence
  Description of Artificial Sequence: Synthetic oligonucleotide
acccaggacccggauguaaua

SEQ ID NO: 1752 - RNA, 21 nt, Artificial Sequence
  Description of Artificial Sequence: Synthetic oligonucleotide
gauguaaucaacaccgcuuua

SEQ ID NO: 1753 - RNA, 21 nt, Artificial Sequence
  Description of Artificial Sequence: Synthetic oligonucleotide
accgcuuugccgauagaauaa

SEQ ID NO: 1754 - RNA, 21 nt, Artificial Sequence
  Description of Artificial Sequence: Synthetic oligonucleotide
auagaauaugguccacuugua

SEQ ID NO: 1755 - RNA, 21 nt, Artificial Sequence
  Description of Artificial Sequence: Synthetic oligonucleotide
ccacuuguggaagaggaagaa

SEQ ID NO: 1756 - RNA, 21 nt, Artificial Sequence
  Description of Artificial Sequence: Synthetic oligonucleotide
ggaagagaaagugccugugaa

SEQ ID NO: 1757 - RNA, 21 nt, Artificial Sequence
  Description of Artificial Sequence: Synthetic oligonucleotide
ugaggcccaaggacccugaga

SEQ ID NO: 1758 - RNA, 21 nt, Artificial Sequence
  Description of Artificial Sequence: Synthetic oligonucleotide
ccuccucuccuggucucucaa

SEQ ID NO: 1759 - RNA, 21 nt, Artificial Sequence
  Description of Artificial Sequence: Synthetic oligonucleotide
ucucucaacaggcaaaacgga

SEQ ID NO: 1760 - RNA, 21 nt, Artificial Sequence
  Description of Artificial Sequence: Synthetic oligonucleotide
ucugccuaccaccuccucuga

SEQ ID NO: 1761 - RNA, 21 nt, Artificial Sequence
  Description of Artificial Sequence: Synthetic oligonucleotide
cuccucuggcaaggcugcaaa

SEQ ID NO: 1762 - RNA, 21 nt, Artificial Sequence
  Description of Artificial Sequence: Synthetic oligonucleotide
cugcaaagaaacccacagcua

SEQ ID NO: 1763 - RNA, 21 nt, Artificial Sequence
  Description of Artificial Sequence: Synthetic oligonucleotide
gcagagaucucuguucgagua

SEQ ID NO: 1764 - RNA, 21 nt, Artificial Sequence
  Description of Artificial Sequence: Synthetic oligonucleotide
guucgagucccuagagggcca

SEQ ID NO: 1765 - RNA, 21 nt, Artificial Sequence
  Description of Artificial Sequence: Synthetic oligonucleotide
gugaauauggcauucucucaa

SEQ ID NO: 1766 - RNA, 21 nt, Artificial Sequence
  Description of Artificial Sequence: Synthetic oligonucleotide
cucucaguccaacccuccuua

SEQ ID NO: 1767 - RNA, 21 nt, Artificial Sequence
  Description of Artificial Sequence: Synthetic oligonucleotide
ccuccuucggaguugcacaaa

SEQ ID NO: 1768 - RNA, 21 nt, Artificial Sequence
  Description of Artificial Sequence: Synthetic oligonucleotide
ccacggauccagaaacaagca

SEQ ID NO: 1769 - RNA, 21 nt, Artificial Sequence
  Description of Artificial Sequence: Synthetic oligonucleotide
caccagcuuguggaacccaaa

SEQ ID NO: 1770 - RNA, 21 nt, Artificial Sequence
  Description of Artificial Sequence: Synthetic oligonucleotide
gaacccaacguacggcuccua

SEQ ID NO: 1771 - RNA, 21 nt, Artificial Sequence
  Description of Artificial Sequence: Synthetic oligonucleotide
uccugguuuacagagaaacca

SEQ ID NO: 1772 - RNA, 21 nt, Artificial Sequence
  Description of Artificial Sequence: Synthetic oligonucleotide
uaauccuauagcaaagaagga

SEQ ID NO: 1773 - RNA, 21 nt, Artificial Sequence
  Description of Artificial Sequence: Synthetic oligonucleotide
agaaggagccacacgacagga

SEQ ID NO: 1774 - RNA, 21 nt, Artificial Sequence
  Description of Artificial Sequence: Synthetic oligonucleotide
agggaagcuguacugucccaa

SEQ ID NO: 1775 - RNA, 21 nt, Artificial Sequence
  Description of Artificial Sequence: Synthetic oligonucleotide
ccaccuaacguugcaacugga

SEQ ID NO: 1776 - RNA, 21 nt, Artificial Sequence
  Description of Artificial Sequence: Synthetic oligonucleotide
acugcuccuagagcccucuua

SEQ ID NO: 1777 - RNA, 21 nt, Artificial Sequence
  Description of Artificial Sequence: Synthetic oligonucleotide
ucgcugacugccaauaugaaa

SEQ ID NO: 1778 - RNA, 21 nt, Artificial Sequence
  Description of Artificial Sequence: Synthetic oligonucleotide
aauaugaaggagguaccucua

SEQ ID NO: 1779 - RNA, 21 nt, Artificial Sequence
  Description of Artificial Sequence: Synthetic oligonucleotide
guaccucuguucaggcuacga

SEQ ID NO: 1780 - RNA, 21 nt, Artificial Sequence
  Description of Artificial Sequence: Synthetic oligonucleotide
gcuacgucacuucccuuguga

SEQ ID NO: 1781 - RNA, 21 nt, Artificial Sequence
  Description of Artificial Sequence: Synthetic oligonucleotide
uugugggaaugucaauuacga

SEQ ID NO: 1782 - RNA, 21 nt, Artificial Sequence
  Description of Artificial Sequence: Synthetic oligonucleotide
ggcuaccagcaacagggcuua

SEQ ID NO: 1783 - RNA, 21 nt, Artificial Sequence
  Description of Artificial Sequence: Synthetic oligonucleotide
uugcccuuagaagccgcuaca

SEQ ID NO: 1784 - RNA, 21 nt, Artificial Sequence
  Description of Artificial Sequence: Synthetic oligonucleotide
uggagcuggucauuacgagga

SEQ ID NO: 1785 - RNA, 21 nt, Artificial Sequence
  Description of Artificial Sequence: Synthetic oligonucleotide
uuacgaggauaccauucugaa

SEQ ID NO: 1786 - RNA, 21 nt, Artificial Sequence
  Description of Artificial Sequence: Synthetic oligonucleotide
cauucugaaaagcaagaauaa

SEQ ID NO: 1787 - RNA, 21 nt, Artificial Sequence
  Description of Artificial Sequence: Synthetic oligonucleotide
agaauagcaugaaccagccua

SEQ ID NO: 1788 - RNA, 21 nt, Artificial Sequence
  Description of Artificial Sequence: Synthetic oligonucleotide
gagcucggucgcacacucaca

SEQ ID NO: 1789 - RNA, 21 nt, Artificial Sequence
  Description of Artificial Sequence: Synthetic oligonucleotide
acacucacuucucuuccuuga

SEQ ID NO: 1790 - RNA, 21 nt, Artificial Sequence
  Description of Artificial Sequence: Synthetic oligonucleotide
gggaucccuaagaccguggaa

SEQ ID NO: 1791 - RNA, 21 nt, Artificial Sequence
  Description of Artificial Sequence: Synthetic oligonucleotide
accguggaggagagagaggca

SEQ ID NO: 1792 - RNA, 21 nt, Artificial Sequence
  Description of Artificial Sequence: Synthetic oligonucleotide
agagaggcaauggcuccuuca

SEQ ID NO: 1793 - RNA, 21 nt, Artificial Sequence
  Description of Artificial Sequence: Synthetic oligonucleotide
gcuccuucacaaaccagagaa

SEQ ID NO: 1794 - RNA, 21 nt, Artificial Sequence
  Description of Artificial Sequence: Synthetic oligonucleotide
gagaccaaaugucacguuuua

SEQ ID NO: 1795 - RNA, 21 nt, Artificial Sequence
  Description of Artificial Sequence: Synthetic oligonucleotide
cacguuuuguuuugugccaaa

SEQ ID NO: 1796 - RNA, 21 nt, Artificial Sequence
  Description of Artificial Sequence: Synthetic oligonucleotide
gccaaccuauuuugaaguaca

SEQ ID NO: 1797 - RNA, 21 nt, Artificial Sequence
  Description of Artificial Sequence: Synthetic oligonucleotide
uguauuuugaaaaugcuuuaa

SEQ ID NO: 1798 - RNA, 21 nt, Artificial Sequence
  Description of Artificial Sequence: Synthetic oligonucleotide
uuuagaaagguuuugagcaua

SEQ ID NO: 1799 - RNA, 21 nt, Artificial Sequence
  Description of Artificial Sequence: Synthetic oligonucleotide
uugagcauggguucauccuaa

SEQ ID NO: 1800 - RNA, 21 nt, Artificial Sequence
  Description of Artificial Sequence: Synthetic oligonucleotide
cauccuauucuuucgaaagaa

SEQ ID NO: 1801 - RNA, 21 nt, Artificial Sequence
  Description of Artificial Sequence: Synthetic oligonucleotide
aaugagugauaaauacaagga

SEQ ID NO: 1802 - RNA, 21 nt, Artificial Sequence
  Description of Artificial Sequence: Synthetic oligonucleotide
auacaaggcccagauguggua

SEQ ID NO: 1803 - RNA, 21 nt, Artificial Sequence
  Description of Artificial Sequence: Synthetic oligonucleotide
gaugugguugcauaagguuua

SEQ ID NO: 1804 - RNA, 21 nt, Artificial Sequence
  Description of Artificial Sequence: Synthetic oligonucleotide
ugcauguuuguuguauacuua

SEQ ID NO: 1805 - RNA, 21 nt, Artificial Sequence
  Description of Artificial Sequence: Synthetic oligonucleotide
uuccuuaugcuucuuucaaaa

SEQ ID NO: 1806 - RNA, 21 nt, Artificial Sequence
  Description of Artificial Sequence: Synthetic oligonucleotide
uuucaaauugugugugcucua

SEQ ID NO: 1807 - RNA, 21 nt, Artificial Sequence
  Description of Artificial Sequence: Synthetic oligonucleotide
cugcuucaauguagucagaaa

SEQ ID NO: 1808 - RNA, 21 nt, Artificial Sequence
  Description of Artificial Sequence: Synthetic oligonucleotide
agucagaauuagcugcuucua

SEQ ID NO: 1809 - RNA, 21 nt, Artificial Sequence
  Description of Artificial Sequence: Synthetic oligonucleotide
ugcuucuauguuucauaguua

SEQ ID NO: 1810 - RNA, 21 nt, Artificial Sequence
  Description of Artificial Sequence: Synthetic oligonucleotide
auguuuccuugccuuguugaa

SEQ ID NO: 1811 - RNA, 21 nt, Artificial Sequence
  Description of Artificial Sequence: Synthetic oligonucleotide
uguggacaugagccauuugaa

SEQ ID NO: 1812 - RNA, 21 nt, Artificial Sequence
  Description of Artificial Sequence: Synthetic oligonucleotide
acggaaauaaaggaguuauua

SEQ ID NO: 1813 - RNA, 21 nt, Artificial Sequence
  Description of Artificial Sequence: Synthetic oligonucleotide
aguuauuuguaaugacuaaaa